(19) United States
(12) Patent Application Publication
(10) Pub. No.: US 2025/0230203 A1
(43) Pub. Date: Jul. 17, 2025

(54) FIBROIN PEPTIDES AND PROTEIN FRAGMENTS COMPOSITIONS

(71) Applicant: EVOLVED BY NATURE, INC., Needham, MA (US)

(72) Inventors: Marios Frantzeskos Sardis, Salem, MA (US); Brian Pink, Somerville, MA (US); Enrico Mortarino, Hickory, NC (US); Svetlana Marukian, Watertown, MA (US); Morgan Cousins, Boston, MA (US); Lior Artzi, Needham, MA (US); Laura R. Muollo, Dracut, MA (US)

(21) Appl. No.: 19/079,960

(22) Filed: Mar. 14, 2025

Related U.S. Application Data
(63) Continuation of application No. PCT/US2023/074411, filed on Sep. 15, 2023.
(60) Provisional application No. 63/407,097, filed on Sep. 15, 2022.
(60) Provisional application No. 63/407,098, filed on Sep. 15, 2022.
(60) Provisional application No. 63/376,542, filed on Sep. 21, 2022.
(60) Provisional application No. 63/376,547, filed on Sep. 21, 2022.
(60) Provisional application No. 63/416,921, filed on Oct. 17, 2022.
(60) Provisional application No. 63/580,982, filed on Sep. 6, 2023.

(51) Int. Cl. C07K 14/435

(52) U.S. Cl. CPC C07K 14/43563

(57) ABSTRACT

The disclosure relates to peptide compositions. e.g., silk fibroin derived peptide compositions.

### CROSS REFERENCE TO RELATED APPLICATIONS

This application is a Continuation of International Application No. PCT/US2023/074411, filed Sep. 15, 2023, which claims the benefit of priority of U.S. Provisional Application No. 63/407,097, filed Sep. 15, 2022, U.S. Provisional Application No. 63/407,098, filed Sep. 15, 2022, U.S. Provisional Application No. 63/376,542, filed Sep. 21, 2022, U.S. Provisional Application No. 63/376,547, filed Sep. 21, 2022, U.S. Provisional Application No. 63/416,921, filed Oct. 17, 2022, and U.S. Provisional Application No. 63/580,982, filed Sep. 6, 2023, each of which is incorporated by reference herein in its entirety.

### REFERENCE TO SEQUENCE LISTING SUBMITTED ELECTRONICALLY

This application contains a Sequence Listing which has been submitted electronically in XML file format and is hereby incorporated by reference in its entirety. The XML file, named 032272-5031-WO.xml, was created on Mar. 12, 2025 and is 58.9 kilobytes in size.

### FIELD

The disclosure relates to peptide compositions, e.g., silk fibroin derived peptide compositions. The present disclosure is also in the field of synthetic fabrics coated with silk fibroin proteins and protein fragments. In further embodiments, the present disclosure is in the field of silk fibroin compositions and methods for stimulating collagen and/or claudin-1 expression.

### BACKGROUND

Silk is a natural polymer produced by a variety of insects and spiders, and comprises a filament core protein, silk fibroin, and a glue-like coating consisting of a non-filamentous protein, sericin.

### SUMMARY

The disclosure provides peptide compositions, e.g., silk fibroin derived peptide compositions.

The disclosure provides a peptide or protein fragment comprising a plurality of amino acids selected from M, R, V, K, T, F, I, L, C, A, Q, Y, N, D, E, G, S, H, P, and W, wherein at least one of the amino acids is modified, substituted, or replaced. In some embodiments, the peptide or protein fragment is a fibroin peptide or protein fragment comprising an amino acid modification, substitution, or replacement of an amino acid from of amino acids selected from M, R, V, K, T, F, I, L, C, A, Q, Y, N, D, E, G, S, H, P, and W. In some embodiments, the fibroin is a fibroin heavy chain, a fibroin light chain, or a fibrohexamerin. In some embodiments, the peptide or protein fragment comprises between about 2 and about 100 amino acids. In some embodiments, the peptide or protein fragment comprises between about 2 and about 25 amino acids. In some embodiments, the peptide or protein fragment comprises between about 25 and about 50 amino acids. In some embodiments, the peptide or protein fragment comprises between about 50 and about 75 amino acids. In some embodiments, the peptide or protein fragment comprises between about 75 and about 100 amino acids. In some embodiments, the peptide or protein fragment comprises between about 100 and about 125 amino acids. In some embodiments, the peptide or protein fragment comprises between about 125 and about 150 amino acids. In some embodiments, the peptide or protein fragment comprises between about 150 and about 200 amino acids. In some embodiments, the peptide or protein fragment comprises between about 200 and about 250, 300, 350, 400, 450, or 500 amino acids. In some embodiments, the peptide or protein fragment comprises between one and five modifications, substitutions, and/or replacements. In some embodiments, the peptide or protein fragment comprises one modification, substitution, and/or replacement. In some embodiments, the peptide or protein fragment comprises two modifications, substitutions, and/or replacements. In some embodiments, the peptide or protein fragment comprises three modifications, substitutions, and/or replacements. In some embodiments, the peptide or protein fragment comprises four modifications, substitutions, and/or replacements. In some embodiments, the peptide or protein fragment comprises five modifications, substitutions, and/or replacements. In some embodiments, the peptide or protein fragment comprises six modifications, substitutions, and/or replacements. In some embodiments, the peptide or protein fragment comprises seven modifications, substitutions, and/or replacements. In some embodiments, the peptide or protein fragment comprises eight modifications, substitutions, and/or replacements. In some embodiments, the peptide or protein fragment comprises nine modifications, substitutions, and/or replacements. In some embodiments, the peptide or protein fragment comprises ten modifications, substitutions, and/or replacements. In some embodiments, a modification, substitution, and/or replacement is selected from an asparagine to aspartic acid modification, substitution, and/or replacement, a glutamine to glutamic acid modification, substitution, and/or replacement, and a methionine to methionine oxide modification, substitution, and/or replacement. In some embodiments, the fibroin is a fibroin heavy chain, and wherein a modification, substitution, and/or replacement is at a position corresponding to any one position from 1 to 5263 of the fibroin heavy chain. In some embodiments, a modification, substitution, and/or replacement is at Q58, M64, N68, N70, N77, M80, N93, M103, Q125, N132, Q139, Q275, N4191, Q5216, and/or N5262 position of fibroin heavy chain. In some embodiments, the fibroin is a fibroin light chain, and wherein a modification, substitution, and/or replacement is at a position corresponding to any one position from 1 to 262 of the fibroin light chain. In some embodiments, a modification, substitution, and/or replacement is at N23, Q24, N28, M69, N105, N108, N118, N136, N138, Q149, N186, N200, Q202, N204, N240, N248, and/or Q255 position of fibroin light chain. In some embodiments, the fibroin is a fibrohexamerin (p25), and wherein a modification, substitution, and/or replacement is at a position corresponding to any one position from 1 to 220 of the fibrohexamerin (p25). In some embodiments, a modification, substitution, and/or replacement is at Q62, N93, M120, N149, N172, N174, and/or N202 position of fibrohexamerin (p25).

The disclosure provides a composition comprising a plurality of peptides or protein fragments, each comprising a plurality of amino acids selected from M, R, V, K, T, F, I, L, C, A, Q, Y, N, D, E, G, S, H, P, and W, wherein at least one of the amino acids is modified, substituted, or replaced. In some embodiments, the plurality of peptides or protein fragments comprises a fibroin peptide or protein fragment comprising an amino acid modification, substitution, or replacement of an amino acid selected from M, R, V, K, T, F, I, L, C, A, Q, Y, N, D, E, G, S, H, P, and W. In some embodiments, the fibroin is a fibroin heavy chain, a fibroin light chain, or a fibrohexamerin. In some embodiments, the peptide or protein fragment comprises between about 2 and about 100 amino acids. In some embodiments, the peptide or protein fragment comprises between about 2 and about 25 amino acids. In some embodiments, the peptide or protein fragment comprises between about 25 and about 50 amino acids. In some embodiments, the peptide or protein fragment comprises between about 50 and about 75 amino acids. In some embodiments, the peptide or protein fragment comprises between about 75 and about 100 amino acids. In some embodiments, the peptide or protein fragment comprises between about 100 and about 125 amino acids. In some embodiments, the peptide or protein fragment comprises between about 125 and about 150 amino acids. In some embodiments, the peptide or protein fragment comprises between about 150 and about 200 amino acids. In some embodiments, the peptide or protein fragment comprises between about 200 and about 250, 300, 350, 400, 450, or 500 amino acids. In some embodiments, the peptide or protein fragment comprises between one and five modifications, substitutions, and/or replacements. In some embodiments, the peptide or protein fragment comprises one modification, substitution, and/or replacement. In some embodiments, the peptide or protein fragment comprises two modifications, substitutions, and/or replacements. In some embodiments, the peptide or protein fragment comprises three modifications, substitutions, and/or replacements. In some embodiments, the peptide or protein fragment comprises four modifications, substitutions, and/or replacements. In some embodiments, the peptide or protein fragment comprises five modifications, substitutions, and/or replacements. In some embodiments, the peptide or protein fragment comprises six modifications, substitutions, and/or replacements. In some embodiments, the peptide or protein fragment comprises seven modifications, substitutions, and/or replacements. In some embodiments, the peptide or protein fragment comprises eight modifications, substitutions, and/or replacements. In some embodiments, the peptide or protein fragment comprises nine modifications, substitutions, and/or replacements. In some embodiments, the peptide or protein fragment comprises ten modifications, substitutions, and/or replacements. In some embodiments, a modification, substitution, and/or replacement is selected from an asparagine to aspartic acid modification, substitution, and/or replacement, a glutamine to glutamic acid modification, substitution, and/or replacement, and a methionine to methionine oxide modification, substitution, and/or replacement. In some embodiments, the fibroin is a fibroin heavy chain, and wherein a modification, substitution, and/or replacement is at a position corresponding to any one position from 1 to 5263 of the fibroin heavy chain. In some embodiments, a modification, substitution, and/or replacement is at Q58, M64, N68, N70, N77, M80, N93, M103, Q125, N132, Q139, Q275, N4191, Q5216, and/or N5262 position of fibroin heavy chain. In some embodiments, the fibroin is a fibroin light chain, and wherein a modification, substitution, and/or replacement is at a position corresponding to any one position from 1 to 262 of the fibroin light chain. In some embodiments, a modification, substitution, and/or replacement is at N23, Q24, N28, M69, N105, N108, N118, N136, N138, Q149, N186, N200, Q202, N204, N240, N248, and/or Q255 position of fibroin light chain. In some embodiments, the fibroin is a fibrohexamerin (p25), and wherein a modification, substitution, and/or replacement is at a position corresponding to any one position from 1 to 220 of the fibrohexamerin (p25). In some embodiments, a modification, substitution, and/or replacement is at Q62, N93, M120, N149, N172, N174, and/or N202 position of fibrohexamerin (p25). In some embodiments, each modification, substitution, and/or replacement is independently ranging in the composition between about 1% to about 99%. In some embodiments, each modification, substitution, and/or replacement is independently ranging in the composition between about 1% to about 10%. In some embodiments, each modification, substitution, and/or replacement is independently ranging in the composition between about 10% to about 20%. In some embodiments, each modification, substitution, and/or replacement is independently ranging in the composition between about 20% to about 30%. In some embodiments, each modification, substitution, and/or replacement is independently ranging in the composition between about 30% to about 40%. In some embodiments, each modification, substitution, and/or replacement is independently ranging in the composition between about 40% to about 50%. In some embodiments, each modification, substitution, and/or replacement is independently ranging in the composition between about 50% to about 60%. In some embodiments, each modification, substitution, and/or replacement is independently ranging in the composition between about 60% to about 70%. In some embodiments, each modification, substitution, and/or replacement is independently ranging in the composition between about 70% to about 80%. In some embodiments, each modification, substitution, and/or replacement is independently ranging in the composition between about 80% to about 90%. In some embodiments, each modification, substitution, and/or replacement is independently ranging in the composition between or about 90% to about 99%. As used herein, a % modification, substitution, and/or replacement is defined as (number of peptide or protein fragments comprising a modification, substitution, and/or replacement at a specific position, divided by the total number of peptide or protein fragments which include the specific position, whether comprising a modification, substitution, and/or replacement, or not)×100.

The disclosure provides a composition comprising a plurality of peptides or protein fragments of fibroin heavy chain, fibroin light chain, and/or fibrohexamerin (p25), the composition comprising one or more fractions, wherein the plurality of peptides or protein fragments comprises a fibroin peptide or protein fragment comprising an amino acid modification, substitution, or replacement. In some embodiments, the plurality of peptides or protein fragments having a weight average molecular weight (M_{w}) selected from between about 1 kDa and about 20 kDa, from between about 20 kDa and about 40 kDa, from between about 40 kDa and about 60 kDa, from between about 60 kDa and about 80 kDa, from between about 80 kDa and about 100 kDa, from between about 100 kDa and about 120 kDa, from between about 120 kDa and about 140 kDa, or from between about 140 kDa and about 160 kDa, from between about 160 kDa and about 180 kDa, from between about 180 kDa and about 200 kDa, or from between about 200 kDa and about 250 kDa, and a polydispersity between 1 and about 1.7, between 1 and less than 3, or between 1 and less than 5. In some embodiments, the plurality of peptides or protein fragments in a fraction having a weight average molecular weight (M_{w}) selected from between about 10 kDa and about 20 kDa, from between about 20 kDa and about 40 kDa, from between about 40 kDa and about 60 kDa, from between about 60 kDa and about 80 kDa, from between about 80 kDa and about 100 kDa, from between about 100 kDa and about 120 kDa, from between about 120 kDa and about 140 kDa, from between about 140 kDa and about 160 kDa, or from between about 160 kDa and about 180 kDa, and a polydispersity between 1 and about 1.1, or 1 and about 1.2, or between 1 and less than 3, or between 1 and less than 5. In some embodiments, the plurality of peptides or protein fragments in a fraction having a weight average molecular weight (M_{w}) selected from between about 10 kDa and about 20 kDa, from between about 20 kDa and about 40 kDa, from between about 40 kDa and about 60 kDa, from between about 60 kDa and about 80 kDa, from between about 80 kDa and about 100 kDa, from between about 100 kDa and about 120 kDa, or from between about 120 kDa and about 140 kDa, and a polydispersity between 1 and about 1.1, or 1 and about 1.2, or between 1 and less than 3, or between 1 and less than 5. In some embodiments, the plurality of peptides or protein fragments in a fraction having a weight average molecular weight (M_{w}) selected from between about 60 kDa and about 80 kDa, from between about 80 kDa and about 100 kDa, or from between about 100 kDa and about 120 kDa, and a polydispersity between 1 and about 1.1, or between 1 and less than 3, or between 1 and less than 5. In some embodiments, the plurality of peptides or protein fragments in a fraction having a weight average molecular weight (M_{w}) selected from between about 10 kDa and about 20 kDa, from between about 20 kDa and about 40 kDa, from between about 40 kDa and about 60 kDa, from between about 60 kDa and about 80 kDa, from between about 80 kDa and about 100 kDa, or from between about 100 kDa and about 110 kDa, and a polydispersity between 1 and about 1.1, or 1 and about 1.2, or between 1 and less than 3, or between 1 and less than 5. In some embodiments, the plurality of peptides or protein fragments in a fraction having a weight average molecular weight (M_{w}) selected from between about 60 kDa and about 80 kDa, from between about 80 kDa and about 100 kDa, from between about 100 kDa and about 120 kDa, or from between about 120 kDa and about 140 kDa, and a polydispersity between 1 and about 1.1, or between 1 and less than 3, or between 1 and less than 5. In some embodiments, the plurality of peptides or protein fragments in a fraction having a weight average molecular weight (M_{w}) selected from between about 20 kDa and about 40 kDa, or from between about 40 kDa and about 60 kDa, and a polydispersity between 1 and about 1.1, or 1 and about 1.2, or between 1 and less than 3, or between 1 and less than 5. In some embodiments, the one or more fractions are selected from AS77, AS78, AS79, AS80, and AS81. In some embodiments, the one or more fractions are selected from AS82, AS83, AS84, AS85, AS86, AS87, AS88, and AS89. In some embodiments, the one or more fractions are selected from AS90, AS91, AS92, AS93, and AS94. In some embodiments, the one or more fractions are selected from AS95, AS96, AS97, AS98, AS99, and AS100. In some embodiments, the plurality of peptides or protein fragments having a weight average molecular weight (M_{w}) selected from between about 40 kDa and about 60 kDa, from between about 60 kDa and about 80 kDa, from between about 80 kDa and about 100 kDa, from between about 100 kDa and about 120 kDa, from between about 120 kDa and about 140 kDa, from between about 140 kDa and about 160 kDa, from between about 160 kDa and about 180 kDa, from between about 180 kDa and about 200 kDa, or from between about 200 kDa and about 220 kDa, and a polydispersity between 1 and about 1.7, or between 1 and less than 3, or between 1 and less than 5. In some embodiments, the plurality of peptides or protein fragments in a fraction having a weight average molecular weight (M_{w}) selected from between about 40 kDa and about 60 kDa, from between about 60 kDa and about 80 kDa, from between about 80 kDa and about 100 kDa, from between about 100 kDa and about 120 kDa, from between about 120 kDa and about 140 kDa, from between about 140 kDa and about 160 kDa, from between about 160 kDa and about 180 kDa, from between about 180 kDa and about 200 kDa, or from between about 200 kDa and about 210 kDa, and a polydispersity between 1 and about 1.2, or 1 and about 1.3, or between 1 and less than 3, or between 1 and less than 5. In some embodiments, the plurality of peptides or protein fragments in a fraction having a weight average molecular weight (M_{w}) selected from between about 40 kDa and about 60 kDa, from between about 60 kDa and about 80 kDa, from between about 80 kDa and about 100 kDa, or from between about 100 kDa and about 110 kDa, and a polydispersity between 1 and about 1.1, or 1 and about 1.2, or between 1 and less than 3, or between 1 and less than 5. In some embodiments, the plurality of peptides or protein fragments in a fraction having a weight average molecular weight (M_{w}) selected from between about 60 kDa and about 80 kDa, from between about 80 kDa and about 100 kDa, from between about 100 kDa and about 120 kDa, from between about 120 kDa and about 140 kDa, from between about 140 kDa and about 160 kDa, from between about 160 kDa and about 180 kDa, from between about 180 kDa and about 200 kDa, or from between about 200 kDa and about 210 kDa, and a polydispersity between 1 and about 1.2, or 1 and about 1.3, or between 1 and less than 3, or between 1 and less than 5. In some embodiments, the one or more fractions are selected from AS101, AS102, AS103, AS104, and AS105. In some embodiments, the one or more fractions are selected from AS106, AS107, AS108, AS109, AS110, and AS111. In some embodiments, an amino acid is selected from M, R, V, K, T, F, I, L, C, A, Q, Y, N, D, E, G, S, H, P, and W. In some embodiments, a peptide or protein fragment comprises between about 2 and about 100 amino acids. In some embodiments, the peptide or protein fragment comprises between about 2 and about 25 amino acids. In some embodiments, the peptide or protein fragment comprises between about 25 and about 50 amino acids. In some embodiments, the peptide or protein fragment comprises between about 50 and about 75 amino acids. In some embodiments, the peptide or protein fragment comprises between about 75 and about 100 amino acids. In some embodiments, the peptide or protein fragment comprises between about 100 and about 125 amino acids. In some embodiments, the peptide or protein fragment comprises between about 125 and about 150 amino acids. In some embodiments, the peptide or protein fragment comprises between about 150 and about 200 amino acids. In some embodiments, the peptide or protein fragment comprises between about 200 and about 250, 300, 350, 400, 450, or 500 amino acids. In some embodiments, a peptide or protein fragment comprises between one and five modifications, substitutions, and/or replacements. In some embodiments, the peptide or protein fragment comprises one modification, substitution, and/or replacement. In some embodiments, the peptide or protein fragment comprises two modifications, substitutions, and/or replacements. In some embodiments, the peptide or protein fragment comprises three modifications, substitutions, and/or replacements. In some embodiments, the peptide or protein fragment comprises four modifications, substitutions, and/or replacements. In some embodiments, the peptide or protein fragment comprises five modifications, substitutions, and/or replacements. In some embodiments, the peptide or protein fragment comprises six modifications, substitutions, and/or replacements. In some embodiments, the peptide or protein fragment comprises seven modifications, substitutions, and/or replacements. In some embodiments, the peptide or protein fragment comprises eight modifications, substitutions, and/or replacements. In some embodiments, the peptide or protein fragment comprises nine modifications, substitutions, and/or replacements. In some embodiments, the peptide or protein fragment comprises ten modifications, substitutions, and/or replacements. In some embodiments, the fibroin is a fibroin heavy chain, and wherein a modification, substitution, and/or replacement is at a position corresponding to any one position from 1 to 5263 of the fibroin heavy chain. In some embodiments, the fibroin is a fibroin light chain, and wherein a modification, substitution, and/or replacement is at a position corresponding to any one position from 1 to 262 of the fibroin light chain. In some embodiments, the fibroin is a fibrohexamerin (p25) chain, and wherein a modification, substitution, and/or replacement is at a position corresponding to any one position from 1 to 220 of the fibrohexamerin (p25) chain. In some embodiments, a modification, substitution, and/or replacement is selected from an asparagine to aspartic acid modification, substitution, and/or replacement, a glutamine to glutamic acid modification, substitution, and/or replacement, and a methionine to methionine oxide modification, substitution, and/or replacement. In some embodiments, a modification, substitution, and/or replacement is at fibroin heavy chain position selected from Q58, M64, N68, N70, N77, M80, N93, M103, Q125, N132, Q139, Q275, N4191, Q5216, and/or N5262. In some embodiments, a modification, substitution, and/or replacement is at fibroin light chain position selected from N23, Q24, N28, M69, N105, N108, N118, N136, N138, Q149, N186, N200, Q202, N204, N240, N248, and/or Q255. In some embodiments, a modification, substitution, and/or replacement is at fibrohexamerin (p25) position selected from Q62, N93, M120, N149, N172, N174, and/or N202. In some embodiments, each modification, substitution, and/or replacement is independently ranging in the composition between about 1% to about 99%. In some embodiments, each modification, substitution, and/or replacement is independently ranging in the composition between about 1% to about 10%. In some embodiments, each modification, substitution, and/or replacement is independently ranging in the composition between about 10% to about 20%. In some embodiments, each modification, substitution, and/or replacement is independently ranging in the composition between about 20% to about 30%. In some embodiments, each modification, substitution, and/or replacement is independently ranging in the composition between about 30% to about 40%. In some embodiments, each modification, substitution, and/or replacement is independently ranging in the composition between about 40% to about 50%. In some embodiments, each modification, substitution, and/or replacement is independently ranging in the composition between about 50% to about 60%. In some embodiments, each modification, substitution, and/or replacement is independently ranging in the composition between about 60% to about 70%. In some embodiments, each modification, substitution, and/or replacement is independently ranging in the composition between about 70% to about 80%. In some embodiments, each modification, substitution, and/or replacement is independently ranging in the composition between about 80% to about 90%. In some embodiments, each modification, substitution, and/or replacement is independently ranging in the composition between or about 90% to about 99%. As used herein, a % modification, substitution, and/or replacement is defined as (number of peptide or protein fragments comprising a modification, substitution, and/or replacement at a specific position, divided by the total number of peptide or protein fragments which include the specific position, whether comprising a modification, substitution, and/or replacement, or not)×100. In some embodiments, a molecular weight is determined by MALS.

The disclosure provides an article comprising one or more peptides or protein fragments disclosed herein, and/or one or more compositions disclosed herein. A composition may include unmodified fibroin peptides and protein fragments. An article is, without limitation, selected from a personal care article disclosed herein, a coated fabric disclosed herein, and/or an article for treating fabrics (e.g., laundry pod) disclosed herein. The disclosure provides an article comprising one or more peptides or protein fragments disclosed herein, and/or one or more compositions disclosed herein, and a substrate or any other support or combination element disclosed herein.

In some embodiments, the disclosure provides a composition disclosed herein comprising a plurality of peptides or protein fragments, e.g., a plurality of fibroin heavy chain peptides or fibroin heavy chain fragments, and comprising a plurality of amino acid modifications, substitutions, and/or replacements, and further comprising one or more ratios of modifications, substitutions, and/or replacements at specific positions selected from:
a ratio of Q58 to M64 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q58 to N68 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q58 to N70 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q58 to N77 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25.
a ratio of Q58 to M80 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q58 to N93 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q58 to M103 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q58 to Q125 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q58 to N132 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q58 to Q139 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q58 to Q275 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q58 to N4191 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q58 to Q5216 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q58 to N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M64 to N68 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M64 to N70 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M64 to N77 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M64 to M80 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M64 to N93 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M64 to M103 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M64 to Q125 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M64 to N132 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M64 to Q139 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M64 to Q275 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M64 to N4191 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M64 to Q5216 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M64 to N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N68 to N70 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N68 to N77 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N68 to M80 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N68 to N93 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N68 to M103 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N68 to Q125 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N68 to N132 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N68 to Q139 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N68 to Q275 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N68 to N4191 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N68 to Q5216 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N68 to N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N70 to N77 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N70 to M80 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N70 to N93 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N70 to M103 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N70 to Q125 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N70 to N132 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N70 to Q139 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N70 to Q275 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N70 to N4191 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N70 to Q5216 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N70 to N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N77 to M80 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N77 to N93 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N77 to M103 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N77 to Q125 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N77 to N132 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N77 to Q139 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N77 to Q275 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N77 to N4191 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N77 to Q5216 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N77 to N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M80 to N93 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M80 to M103 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M80 to Q125 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M80 to N132 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M80 to Q139 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M80 to Q275 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M80 to N4191 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M80 to Q5216 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M80 to N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N93 to M103 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N93 to Q125 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N93 to N132 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N93 to Q139 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N93 to Q275 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N93 to N4191 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N93 to Q5216 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N93 to N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M103 to Q125 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M103 to N132 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M103 to Q139 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M103 to Q275 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M103 to N4191 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M103 to Q5216 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M103 to N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q125 to N132 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q125 to Q139 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q125 to Q275 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q125 to N4191 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q125 to Q5216 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q125 to N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N132 to Q139 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N132 to Q275 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N132 to N4191 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N132 to Q5216 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N132 to N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q139 to Q275 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q139 to N4191 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q139 to Q5216 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q139 to N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q275 to N4191 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q275 to Q5216 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q275 to N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N4191 to Q5216 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N4191 to N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q5216 to N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;

In some embodiments, the disclosure provides a composition comprising a plurality of peptides or protein fragments, e.g., a plurality of fibroin heavy chain and light chain peptides or fibroin heavy chain and light chain fragments, and comprising a plurality of amino acid modifications, substitutions, and/or replacements, and further comprising one or more ratios of modifications, substitutions, and/or replacements at specific positions selected from:
a ratio of heavy chain Q58 to light chain N23, Q24, N28, M69, N105, N108, N118, N136, N138, Q149, N186, N200, Q202, N204, N240, N248, or Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain M64 to light chain N23, Q24, N28, M69, N105, N108, N118, N136, N138, Q149, N186, N200, Q202, N204, N240, N248, or Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain N68 to light chain N23, Q24, N28, M69, N105, N108, N118, N136, N138, Q149, N186, N200, Q202, N204, N240, N248, or Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain N70 to light chain N23, Q24, N28, M69, N105, N108, N118, N136, N138, Q149, N186, N200, Q202, N204, N240, N248, or Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain N77 to light chain N23, Q24, N28, M69, N105, N108, N118, N136, N138, Q149, N186, N200, Q202, N204, N240, N248, or Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain M80 to light chain N23, Q24, N28, M69, N105, N108, N118, N136, N138, Q149, N186, N200, Q202, N204, N240, N248, or Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain N93 to light chain N23, Q24, N28, M69, N105, N108, N118, N136, N138, Q149, N186, N200, Q202, N204, N240, N248, or Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain M103 to light chain N23, Q24, N28, M69, N105, N108, N118, N136, N138, Q149, N186, N200, Q202, N204, N240, N248, or Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain Q125 to light chain N23, Q24, N28, M69, N105, N108, N118, N136, N138, Q149, N186, N200, Q202, N204, N240, N248, or Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain N132 to light chain N23, Q24, N28, M69, N105, N108, N118, N136, N138, Q149, N186, N200, Q202, N204, N240, N248, or Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain Q139 to light chain N23, Q24, N28, M69, N105, N108, N118, N136, N138, Q149, N186, N200, Q202, N204, N240, N248, or Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain Q275 to light chain N23, Q24, N28, M69, N105, N108, N118, N136, N138, Q149, N186, N200, Q202, N204, N240, N248, or Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain N4191 to light chain N23, Q24, N28, M69, N105, N108, N118, N136, N138, Q149, N186, N200, Q202, N204, N240, N248, or Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain Q5216 to light chain N23, Q24, N28, M69, N105, N108, N118, N136, N138, Q149, N186, N200, Q202, N204, N240, N248, or Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain N5262 to light chain N23, Q24, N28, M69, N105, N108, N118, N136, N138, Q149, N186, N200, Q202, N204, N240, N248, or Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25.

In some embodiments, the disclosure provides a composition comprising a plurality of peptides or protein fragments, e.g., a plurality of fibroin heavy chain and p25 peptides or fibroin heavy chain and p25 fragments, and comprising a plurality of amino acid modifications, substitutions, and/or replacements, comprising one or more ratios of modifications, substitutions, and/or replacements at specific positions selected from:
a ratio of heavy chain Q58 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain M64 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain N68 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain N70 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain N77 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain M80 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain N93 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain M103 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain Q125 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain N132 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain Q139 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain Q275 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain N4191 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain Q5216 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of heavy chain N5262 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25.

In some embodiments, the disclosure provides a composition comprising a plurality of peptides or protein fragments, e.g., a plurality of light chain fibroin peptides or fibroin fragments, and comprising a plurality of amino acid modifications, substitutions, and/or replacements, comprising one or more ratios of modifications, substitutions, and/or replacements at specific positions selected from:
a ratio of N23 to Q24 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q24 to N28 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q24 to M69 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q24 to N105 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q24 to N108 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q24 to N118 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q24 to N136 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q24 to N138 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q24 to Q149 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q24 to N186 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q24 to N200 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q24 to Q202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q24 to N204 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q24 to N240 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q24 to N248 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q24 to Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N28 to M69 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N28 to N105 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N28 to N108 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N28 to N118 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N28 to N136 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N28 to N138 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N28 to Q149 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N28 to N186 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N28 to N200 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N28 to Q202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N28 to N204 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N28 to N240 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N28 to N248 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N28 to Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M69 to N105 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M69 to N108 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M69 to N118 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M69 to N136 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M69 to N138 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M69 to Q149 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M69 to N186 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M69 to N200 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M69 to Q202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M69 to N204 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M69 to N240 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M69 to N248 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M69 to Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N105 to N108 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N105 to N118 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N105 to N136 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N105 to N138 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N105 to Q149 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N105 to N186 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N105 to N200 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N105 to Q202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N105 to N204 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N105 to N240 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N105 to N248 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N105 to Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N108 to N118 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N108 to N136 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N108 to N138 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N108 to Q149 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N108 to N186 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N108 to N200 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N108 to Q202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N108 to N204 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N108 to N240 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N108 to N248 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N108 to Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N118 to N136 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N118 to N138 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N118 to Q149 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N118 to N186 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N118 to N200 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N118 to Q202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N118 to N204 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N118 to N240 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N118 to N248 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N118 to Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N136 to N138 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N136 to Q149 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N136 to N186 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N136 to N200 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N136 to Q202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N136 to N204 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N136 to N240 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N136 to N248 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N136 to Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N138 to Q149 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N138 to N186 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N138 to N200 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N138 to Q202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N138 to N204 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N138 to N240 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N138 to N248 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N138 to Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q149 to N186 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q149 to N200 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q149 to Q202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q149 to N204 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q149 to N240 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q149 to N248 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q149 to Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N186 to N200 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N186 to Q202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N186 to N204 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N186 to N240 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N186 to N248 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N186 to Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N200 to Q202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N200 to N204 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N200 to N240 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N200 to N248 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N200 to Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q202 to N204 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q202 to N240 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q202 to N248 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q202 to Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N204 to N240 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N204 to N248 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N204 to Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N240 to N248 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N240 to Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N248 to Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;

In some embodiments, the disclosure provides a composition comprising a plurality of peptides or protein fragments, e.g., a plurality of fibroin light chain and heavy chain peptides or fibroin light chain and heavy chain fragments, and comprising a plurality of amino acid modifications, substitutions, and/or replacements, and further comprising one or more ratios of modifications, substitutions, and/or replacements at specific positions selected from:
a ratio of light chain N23 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain Q24 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain N28 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain M69 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain N105 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain N108 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain N118 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain N136 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain N138 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain Q149 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain N186 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain N200 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain Q202 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain N204 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain N240 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain N248 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain Q255 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25.

In some embodiments, the disclosure provides a composition comprising a plurality of peptides or protein fragments, e.g., a plurality of fibroin light chain and p25 peptides or fibroin light chain and p25 fragments, and comprising a plurality of amino acid modifications, substitutions, and/or replacements, comprising one or more ratios of modifications, substitutions, and/or replacements at specific positions selected from:
a ratio of light chain N23 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain Q24 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain N28 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain M69 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain N105 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain N108 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain N118 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain N136 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain N138 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain Q149 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain N186 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain N200 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain Q202 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain N204 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain N240 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain N248 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of light chain Q255 to p25 Q62, N93, M120, N149, N172, N174, or N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25.

In some embodiments, the disclosure provides a composition comprising a plurality of peptides or protein fragments, e.g., a plurality of p25 fibroin peptides or fibroin fragments, and comprising a plurality of amino acid modifications, substitutions, and/or replacements, comprising one or more ratios of modifications, substitutions, and/or replacements at specific positions selected from:
a ratio of Q62 to N93 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q62 to M120 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q62 to N149 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q62 to N172 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q62 to N174 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of Q62 to N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N93 to M120 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N93 to N149 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N93 to N172 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N93 to N174 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N93 to N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M120 to N149 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M120 to N172 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M120 to N174 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of M120 to N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N149 to N172 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N149 to N174 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N149 to N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N172 to N174 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N172 to N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of N174 to N202 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25.

In some embodiments, the disclosure provides a composition comprising a plurality of peptides or protein fragments, e.g., a plurality of p25 and heavy chain peptides or p25 and heavy chain fragments, and comprising a plurality of amino acid modifications, substitutions, and/or replacements, comprising one or more ratios of modifications, substitutions, and/or replacements at specific positions selected from:
a ratio of p25 Q62 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of p25 N93 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of p25 M120 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of p25 N149 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of p25 N172 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of p25 N174 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of p25 N202 to heavy chain Q58, M64, N68, N70, N77, M80, N93, M103, Q125, N132, Q139, Q275, N4191, Q5216, or N5262 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25.

In some embodiments, the disclosure provides a composition comprising a plurality of peptides or protein fragments, e.g., a plurality of p25 and light chain peptides or fibroin light chain and light chain fragments, and comprising a plurality of amino acid modifications, substitutions, and/or replacements, and further comprising one or more ratios of modifications, substitutions, and/or replacements at specific positions selected from:
a ratio of p25 Q62 to light chain N23, Q24, N28, M69, N105, N108, N118, N136, N138, Q149, N186, N200, Q202, N204, N240, N248, or Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of p25 N93 to light chain N23, Q24, N28, M69, N105, N108, N118, N136, N138, Q149, N186, N200, Q202, N204, N240, N248, or Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of p25 M120 to light chain N23, Q24, N28, M69, N105, N108, N118, N136, N138, Q149, N186, N200, Q202, N204, N240, N248, or Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of p25 N149 to light chain N23, Q24, N28, M69, N105, N108, N118, N136, N138, Q149, N186, N200, Q202, N204, N240, N248, or Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of p25 N172 to light chain N23, Q24, N28, M69, N105, N108, N118, N136, N138, Q149, N186, N200, Q202, N204, N240, N248, or Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of p25 N174 to light chain N23, Q24, N28, M69, N105, N108, N118, N136, N138, Q149, N186, N200, Q202, N204, N240, N248, or Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25;
a ratio of p25 N202 to light chain N23, Q24, N28, M69, N105, N108, N118, N136, N138, Q149, N186, N200, Q202, N204, N240, N248, or Q255 modifications, substitutions, and/or replacements of about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, about 1:20, or about 1:25.

The disclosure provides an article comprising a fabric and a coating, wherein the coating comprises a surfactant and/or emulsifier system, and silk fibroin fragments having an average weight average molecular weight selected from between about 1 kDa and about 5 kDa, from between about 5 kDa and about 10 kDa, from between about 6 kDa and about 17 kDa, from between about 10 kDa and about 15 kDa, from between about 14 kDa and about 30 kDa, from between about 15 kDa and about 20 kDa, from between about 17 kDa and about 39 kDa, from between about 20 kDa and about 25 kDa, from between about 25 kDa and about 30 kDa, from between about 30 kDa and about 35 kDa, from between about 35 kDa and about 40 kDa, from between about 39 kDa and about 54 kDa, from between about 39 kDa and about 80 kDa, from between about 40 kDa and about 45 kDa, from between about 45 kDa and about 50 kDa, from between about 50 kDa and about 55 kDa, from between about 55 kDa and about 60 kDa, from between about 60 kDa and about 100 kDa, or from between about 80 kDa and about 144 kDa, and a polydispersity ranging from 1 to about 5. In some embodiments, the coating comprises fibroin peptides and/or fibroin fragments disclosed herein, e.g., and without limitation, fibroin peptides and/or protein fragments wherein at least one of the amino acids is modified, substituted, or replaced as disclosed herein. In some embodiments, the silk fibroin fragments have a polydispersity from 1 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, or from about 4.5 to about 5.0. In some embodiments, the silk fibroin fragments have a polydispersity from about 1.5 to about 3.0. In some embodiments, the silk fibroin fragments comprise one or more of low molecular weight silk fibroin fragments and medium molecular weight silk fibroin fragments. In some embodiments, the article further comprises about 0.01% (w/w) to about 10% (w/w) sericin relative to the silk fibroin fragments. In some embodiments, the fabric comprises one or more of polyester, polyamide, polyaramid, polytetrafluoroethylene, polyethylene, polypropylene, polyurethane, silicone, mixtures of polyurethane and polyethyleneglycol, ultrahigh molecular weight polyethylene, high-performance polyethylene, nylon, LYCRA (polyester-polyurethane copolymer, also known as SPANDEX and elastomer), or a mixture thereof. In some embodiments, the coating further comprises one or more of a wetting agent, an anti-foaming agent, a softener, a wicking agent, and an anti-microbial. In some embodiments, the w/w ratio of silk fibroin fragments to the surfactant and/or emulsifier system in the coating is about 99:1, about 98:2, about 97:3, about 96:4, about 95:5, about 94:6, about 93:7, about 92:8, about 91:9, about 90:10, about 89:11, about 88:12, about 87:13, about 86:14, about 85:15, about 84:16, about 83:17, about 82:18, about 81:19, about 80:20, about 79:21, about 78:22, about 77:23, about 76:24, about 75:25, about 74:26, about 73:27, about 72:28, about 71:29, about 70:30, about 69:31, about 68:32, about 67:33, about 66:34, about 65:35, about 64:36, about 63:37, about 62:38, about 61:39, about 60:40, about 59:41, about 58:42, about 57:43, about 56:44, about 55:45, about 54:46, about 53:47, about 52:48, about 51:49, about 50:50, about 49:51, about 48:52, about 47:53, about 46:54, about 45:55, about 44:56, about 43:57, about 42:58, about 41:59, about 40:60, about 39:61, about 38:62, about 37:63, about 36:64, about 35:65, about 34:66, about 33:67, about 32:68, about 31:69, about 30:70, about 29:71, about 28:72, about 27:73, about 26:74, about 25:75, about 24:76, about 23:77, about 22:78, about 21:79, about 20:80, about 19:81, about 18:82, about 17:83, about 16:84, about 15:85, about 14:86, about 13:87, about 12:88, about 11:89, about 10:90, about 9:91, about 8:92, about 7:93, about 6:94, about 5:95, about 4:96, about 3:97, about 2:98, or about 1:99. In some embodiments, the w/w ratio of silk fibroin fragments to the surfactant and/or emulsifier in the coating is about 1:1, about 1:2, about 1:4, about 1:8, about 1:16, or about 1:32. In some embodiments, the w/w ratio of silk fibroin fragments to the surfactant and/or emulsifier system in the coating is about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24, about 1:25, about 1:26, about 1:27, about 1:28, about 1:29, about 1:30, about 1:31, or about 1:32. In some embodiments, the surfactant and/or emulsifier system comprises one or more of polyoxyethylene sorbitan monooleate, polyoxyethylene sorbitan trioleate, polyoxyethylene castor oil, and any combination thereof. In some embodiments, the surfactant and/or emulsifier system comprises one or more of polyoxyethylene (10-30) sorbitan monooleate, polyoxyethylene (10-30) sorbitan trioleate, polyoxyethylene (10-50) castor oil, and any combination thereof. In some embodiments, the surfactant and/or emulsifier system comprises one or more of polyoxyethylene (20) sorbitan monooleate, polyoxyethylene (20) sorbitan trioleate, polyoxyethylene (29) castor oil, and any combination thereof. In some embodiments, the surfactant and/or emulsifier system comprises one or more of polyoxyethylene (20) sorbitan monolaurate, polyoxyethylene (20) sorbitan monopalmitate, polyoxyethylene (20) sorbitan monostearate, polyoxyethylene (20) sorbitan tristearate, and any combination thereof. In some embodiments, the surfactant and/or emulsifier system comprises one or more of a sorbitan mono fatty acid, a sorbitan tri fatty acid, a castor oil, and any combination thereof. In some embodiments, the surfactant and/or emulsifier system comprises one or more of coco glucoside, decyl glucoside, lauryl glucoside, sucrose cocoate, capryl/caprylyl glucoside, caprylyl/capryl glucoside, and any combination thereof. In some embodiments, the surfactant and/or emulsifier system has an HLB between about 11 and about 13.50. In some embodiments, the surfactant and/or emulsifier system has an HLB between about 11 and about 11.50, between about 11.50 and about 12, between about 12 and about 12.50, between about 12.50 and about 13, or between about 13 and about 13.50. In some embodiments, the article has an improved moisture management comparative to a similar article comprising a similar fabric but no coating. In some embodiments, moisture management is assessed by a water absorbency test, a vertical wicking test, or a dry rate test. In some embodiments, the article has an improved drapability comparative to a similar article comprising a similar fabric but no coating. In some embodiments, the article has an improved smoothness comparative to a similar article comprising a similar fabric but no coating. In some embodiments, the article has an improved hand feel comparative to a similar article comprising a similar fabric but no coating. In some embodiments, the article has a lower charge density at a given pH value comparative to a similar article comprising a similar fabric but no coating.

The disclosure provides a method of making a silk fibroin coated fabric, comprising: applying to the fabric a solution comprising a surfactant and/or emulsifier system; applying to the fabric a silk fibroin fragments solution; and drying the fabric. In some embodiments, the method comprises the use of modified silk fragments disclosed herein, e.g., and without limitation, fibroin peptides and/or protein fragments wherein at least one of the amino acids is modified, substituted, or replaced as disclosed herein. The disclosure also provides a method of making a silk fibroin coated fabric, comprising: applying to the fabric a solution comprising a surfactant and/or emulsifier system and silk fibroin fragments; and drying the fabric. In some embodiments, the concentration of the silk fibroin fragments in a solution ranges from 0.01 g/L to about 100 g/L. In some embodiments, the concentration of the surfactant and/or emulsifier system in a solution ranges from 0.01 g/L to about 100 g/L. In some embodiments, the silk fibroin fragments have an average weight average molecular weight selected from between about 1 kDa and about 5 kDa, from between about 5 kDa and about 10 kDa, from between about 6 kDa and about 17 kDa, from between about 10 kDa and about 15 kDa, from between about 14 kDa and about 30 kDa, from between about 15 kDa and about 20 kDa, from between about 17 kDa and about 39 kDa, from between about 20 kDa and about 25 kDa, from between about 25 kDa and about 30 kDa, from between about 30 kDa and about 35 kDa, from between about 35 kDa and about 40 kDa, from between about 39 kDa and about 54 kDa, from between about 39 kDa and about 80 kDa, from between about 40 kDa and about 45 kDa, from between about 45 kDa and about 50 kDa, from between about 50 kDa and about 55 kDa, from between about 55 kDa and about 60 kDa, from between about 60 kDa and about 100 kDa, or from between about 80 kDa and about 144 kDa, and a polydispersity ranging from 1 to about 5. In some embodiments, the silk fibroin fragments have a polydispersity from 1 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, or from about 4.5 to about 5.0. In some embodiments, the silk fibroin fragments have a polydispersity from about 1.5 to about 3.0. In some embodiments, the silk fibroin fragments comprise one or more of low molecular weight silk fibroin fragments and medium molecular weight silk fibroin fragments. In some embodiments, a solution further comprises about 0.01% (w/w) to about 10% (w/w) sericin relative to the silk fibroin fragments. In some embodiments, the fabric comprises one or more of polyester, polyamide, polyaramid, polytetrafluoroethylene, polyethylene, polypropylene, polyurethane, silicone, mixtures of polyurethane and polyethyleneglycol, ultrahigh molecular weight polyethylene, high-performance polyethylene, nylon, LYCRA (polyester-polyurethane copolymer, also known as SPANDEX and elastomer), or a mixture thereof. In some embodiments, a solution further comprises one or more of a wetting agent, an anti-foaming agent, a softener, a wicking agent, and an anti-microbial. In some embodiments, the w/w ratio of silk fibroin fragments to the surfactant and/or emulsifier system is about 99:1, about 98:2, about 97:3, about 96:4, about 95:5, about 94:6, about 93:7, about 92:8, about 91:9, about 90:10, about 89:11, about 88:12, about 87:13, about 86:14, about 85:15, about 84:16, about 83:17, about 82:18, about 81:19, about 80:20, about 79:21, about 78:22, about 77:23, about 76:24, about 75:25, about 74:26, about 73:27, about 72:28, about 71:29, about 70:30, about 69:31, about 68:32, about 67:33, about 66:34, about 65:35, about 64:36, about 63:37, about 62:38, about 61:39, about 60:40, about 59:41, about 58:42, about 57:43, about 56:44, about 55:45, about 54:46, about 53:47, about 52:48, about 51:49, about 50:50, about 49:51, about 48:52, about 47:53, about 46:54, about 45:55, about 44:56, about 43:57, about 42:58, about 41:59, about 40:60, about 39:61, about 38:62, about 37:63, about 36:64, about 35:65, about 34:66, about 33:67, about 32:68, about 31:69, about 30:70, about 29:71, about 28:72, about 27:73, about 26:74, about 25:75, about 24:76, about 23:77, about 22:78, about 21:79, about 20:80, about 19:81, about 18:82, about 17:83, about 16:84, about 15:85, about 14:86, about 13:87, about 12:88, about 11:89, about 10:90, about 9:91, about 8:92, about 7:93, about 6:94, about 5:95, about 4:96, about 3:97, about 2:98, or about 1:99. In some embodiments, the w/w ratio of silk fibroin fragments to the surfactant and/or emulsifier system is about 1:1, about 1:2, about 1:4, about 1:8, about 1:16, or about 1:32. In some embodiments, the w/w ratio of silk fibroin fragments to the surfactant and/or emulsifier system is about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24, about 1:25, about 1:26, about 1:27, about 1:28, about 1:29, about 1:30, about 1:31, or about 1:32. In some embodiments, the surfactant and/or emulsifier system comprises one or more of polyoxyethylene sorbitan monooleate, polyoxyethylene sorbitan trioleate, polyoxyethylene castor oil, and any combination thereof. In some embodiments, the surfactant and/or emulsifier system comprises one or more of polyoxyethylene (10-30) sorbitan monooleate, polyoxyethylene (10-30) sorbitan trioleate, polyoxyethylene (10-50) castor oil, and any combination thereof. In some embodiments, the surfactant and/or emulsifier system comprises one or more of polyoxyethylene (20) sorbitan monooleate, polyoxyethylene (20) sorbitan trioleate, polyoxyethylene (29) castor oil, and any combination thereof. In some embodiments, the surfactant and/or emulsifier system comprises one or more of polyoxyethylene (20) sorbitan monolaurate, polyoxyethylene (20) sorbitan monopalmitate, polyoxyethylene (20) sorbitan monostearate, polyoxyethylene (20) sorbitan tristearate, and any combination thereof. In some embodiments, the surfactant and/or emulsifier system comprises one or more of a sorbitan mono fatty acid, a sorbitan tri fatty acid, a castor oil, and any combination thereof. In some embodiments, the surfactant and/or emulsifier system comprises one or more of coco glucoside, decyl glucoside, lauryl glucoside, sucrose cocoate, capryl/caprylyl glucoside, caprylyl/capryl glucoside, and any combination thereof. In some embodiments, the surfactant and/or emulsifier system has an HLB between about 11 and about 13.50. In some embodiments, the surfactant and/or emulsifier system has an HLB between about 11 and about 11.50, between about 11.50 and about 12, between about 12 and about 12.50, between about 12.50 and about 13, or between about 13 and about 13.50. In some embodiments, the drying comprises heating. In some embodiments, the pH of a solution is acidic. In some embodiments, the pH of a solution is between about 3.5 and about 4, between about 4 and about 4.5, between about 4.5 and about 5, between about 5 and about 5.5, or between about 5.5 and about 6.

The disclosure also provides an article prepared by a method described herein, including, without limitation, by using a modified silk fragment disclosed herein, e.g., and without limitation, fibroin peptides and/or protein fragments wherein at least one of the amino acids is modified, substituted, or replaced as disclosed herein. In some embodiments, the article has an improved moisture management comparative to a similar article comprising a similar fabric but no coating. In some embodiments, moisture management is assessed by a water absorbency test, a vertical wicking test, or a dry rate test. In some embodiments, the article has an improved drapability comparative to a similar article comprising a similar fabric but no coating. In some embodiments, the article has an improved smoothness comparative to a similar article comprising a similar fabric but no coating. In some embodiments, the article has an improved hand feel comparative to a similar article comprising a similar fabric but no coating. In some embodiments, the article has a lower charge density at a given pH value comparative to a similar article comprising a similar fabric but no coating.

In some embodiments, the disclosure further provides personal care and/or cosmetic formulations and/or compositions comprising silk fibroin fragments having an average weight average molecular weight selected from between about 1 kDa and about 5 kDa, between about 5 kDa and about 10 kDa, between about 6 kDa and about 17 kDa, between about 10 kDa and about 15 kDa, between about 15 kDa and about 20 kDa, between about 14 kDa and about 30 kDa, between about 17 kDa and about 39 kDa, between about 20 kDa and about 25 kDa, between about 25 kDa and about 30 kDa, between about 30 kDa and about 35 kDa, between about 35 kDa and about 40 kDa, between about 39 kDa and about 54 kDa, between about 39 kDa and about 80 kDa, between about 40 kDa and about 45 kDa, between about 45 kDa and about 50 kDa, between about 60 kDa and about 100 kDa, and between about 80 kDa and about 144 kDa, and a polydispersity between 1 and about 5, and without limitation, can also comprise modified silk fragments disclosed herein, e.g., and without limitation, fibroin peptides and/or protein fragments wherein at least one of the amino acids is modified, substituted, or replaced as disclosed herein. In some embodiments, the composition further comprises 0 to 500 ppm lithium bromide. In some embodiments, the composition further comprises 0 to 500 ppm sodium carbonate. In some embodiments, the silk fibroin fragments have a polydispersity between 1 and about 1.5. In some embodiments, the silk fibroin fragments have a polydispersity between about 1.5 and about 2.0. In some embodiments, the silk fibroin fragments have a polydispersity between about 1.5 and about 3.0. In some embodiments, the silk fibroin fragments have a polydispersity between about 2.0 and about 2.5. In some embodiments, the silk fibroin fragments have a polydispersity between about 2.5 and about 3.0. In some embodiments, the silk fibroin fragments are present in the composition at about 0.001 wt. % to about 10.0 wt. % relative to the total weight of the composition. In some embodiments, the composition further comprises about 0.001% (w/w) to about 10% (w/w) sericin relative to the total weight of the composition. In some embodiments, the composition further comprises about 0.001% (w/w) to about 10% (w/w) sericin relative to the silk fibroin fragments. In some embodiments, the silk fibroin fragments do not spontaneously or gradually gelate and do not visibly change in color or turbidity when in an aqueous solution for at least 10 days prior to formulation into the composition. In some embodiments, the silk fibroin fragments are present in the composition at about 0.01 wt. % to about 10.0 wt. % relative to the total weight of the composition. In some embodiments, the silk fibroin fragments are present in the composition at about 0.01 wt. % to about 1.0 wt. % relative to the total weight of the composition. In some embodiments, the silk fibroin fragments are present in the composition at about 1.0 wt. % to about 2.0 wt. % relative to the total weight of the composition. In some embodiments, the silk fibroin fragments are present in the composition at about 2.0 wt. % to about 3.0 wt. % relative to the total weight of the composition. In some embodiments, the silk fibroin fragments are present in the composition at about 3.0 wt. % to about 4.0 wt. % relative to the total weight of the composition. In some embodiments, the silk fibroin fragments are present in the composition at about 4.0 wt. % to about 5.0 wt. % relative to the total weight of the composition. In some embodiments, the silk fibroin fragments are present in the composition at about 5.0 wt. % to about 6.0 wt. % relative to the total weight of the composition.

In some embodiments, the composition is formulated as a topical composition. In some embodiments, the composition further comprises a pharmaceutically acceptable carrier. In some embodiments, the composition further comprises a dermatologically acceptable carrier. In some embodiments, the pharmaceutically acceptable carrier comprises one or more of a suspension, an emulsion, a powder, a solution, a dispersion, or an elixir. In some embodiments, the pharmaceutically acceptable carrier comprises or is formulated as one or more of a gel, a jelly, a cream, a lotion, a foam, a slurry, an ointment, an oil, a paste, a suppository, a spray, a semisolid composition, a solid composition, a stick, or a mousse. In some embodiments, the pharmaceutically acceptable carrier comprises one or more of sesame oil, corn oil, cottonseed oil, or peanut oil. In some embodiments, the pharmaceutically acceptable carrier comprises one or more of mannitol or dextrose. In some embodiments, the pharmaceutically acceptable carrier comprises free and/or uncrosslinked hyaluronic acid. In some embodiments, the pharmaceutically acceptable carrier comprises one or more of aliphatic oil, a fatty alcohol, a fatty acid, a glyceride, an acylglycerol, and a phospholipid. In some embodiments, the pharmaceutically acceptable carrier comprises one or more of a monoglyceride, a diglyceride, or a triglyceride. In some embodiments, the pharmaceutically acceptable carrier comprises an aqueous phase. In some embodiments, the pharmaceutically acceptable carrier comprises an oil-in-water emulsion or a water-in-oil emulsion. In some embodiments, the pharmaceutically acceptable carrier comprises one or more of a hydrocarbon oil, a fatty acid, a fatty oil, a fatty acid ester, or a cationic quaternary ammonium salt.

### BRIEF DESCRIPTION OF THE DRAWINGS

The presently disclosed embodiments will be further explained with reference to the attached drawings. The drawings shown are not necessarily to scale, with emphasis instead generally being placed upon illustrating the principles of the presently disclosed embodiments.

FIG. 1. Ion Exchange Fractionation Schemes for the isolation of the populations that constitute Low and Mid Skid silk/modified polypeptide compositions. Low and Mid Skid silk/modified polypeptide compositions contains silk/modified polypeptides that are negatively, positively charged, or neutral. Using Q anion exchange chromatography (A) these populations were isolated.

FIG. 2. Chromatogram of Low Skid silk/modified polypeptide composition loaded in a Q-Sepharose HP column (Cytiva). The flow through contains the silk/modified polypeptides that do not get captured in the column and are the depleted in negatively charged amino acids. After the column is loaded with Low or Mid Skid silk/modified polypeptide compositions and the flow through is collected, the column is washed until the UV-280 absorbance becomes less than 200 AU. The captured negatively charged silk/modified polypeptides are eluted with high salt concentration (1M NaCl) and constitute AS11 and AS22. The chromatography is performed in Tris-containing buffers but the flow through and the Q-elution were finally dialyzed in water.

FIG. 3. Analytical Size Exclusion Chromatography of Low, Mid Skid silk/modified silk compositions and their constituent AS compositions. Average molecular weight in kDa and polydispersity measurements are shown.

FIG. 4A-FIG. 4B. Analytical Size Exclusion Chromatography of the Low and Mid skid silk/modified peptide compositions and their components (see table 1 for more details). FIG. 4A, Molecular weight of the various Activated Silk new compositions described in this study. FIG. 4B, Polydispersity (PDI) of the various Activated Silk new compositions described in this study. AS24 reconstitutes the average molecular weight and polydispersity of the Low skid silk/modified peptide composition and it consists of 50% AS12 and 50% AS22 (see table 1 for details). AS6 reconstitutes the average molecular weight and polydispersity of the Mid skid silk/modified peptide composition and it consists of 50% AS1 and 50% AS11 (see table 1 for details).

FIG. 5. Isoelectric Focusing Electrophoresis of Low Skid silk/modified polypeptide compositions. Lanes 2, 7; Low Skid silk different amounts loaded. Lanes 3, 5, 8, 10; AS12 silk, different preparations different amounts loaded. Lanes 4, 6, 9, 11; AS22 silk, different preparations different amounts loaded.

FIG. 6A-FIG. 6B. Self-assembly reactions of the of the Low and Mid skid silk/modified peptide compositions and their components (see table 1 for more details). Both graphs depict the kinetic parameters of gel formation during self-assembly of silk. On graph A calculation of the three self-assembly kinetic parameters is shown, t0.5, Amar and SARF. For more details look at the text.

FIG. 7A-FIG. 7C. Self-assembly kinetics of the Low and Mid skid silk/modified peptide compositions and their components (see table 1 for more details). FIG. 7A, the Self-assembly Rate Factor shows how fast the self-assembly reaction proceeds once it is initiated and the self-assembly nuclei are organized. FIG. 7B, Maximum Gel Yield shows how dense the silk gel is after self-assembly is complete. FIG. 7C, Time required for the self-assembly reaction to produce half of the maximum gel amount.

FIG. 8. The Low and Mid skid silk/modified peptide compositions and their components (see table 1 for more details). The Self Assembly Factor reflects the average propensity of silk to self-assemble and form gels.

FIG. 9 is a graph of Weight average molecular weight (i.e., average molecular weight average or average MW) using Size exclusion chromatography with a refractive index detector (SEC-RI) plotted as a function of time for solubilized fibroin in 9.3 M LiBr at 100° C.-103° C. (i.e., 3 degree Celsius temperature gradient between 100° C. and 103° C.).

FIG. 10 is a graph of weight average molecular weight (i.e., average molecular weight average or average MW) using Size exclusion chromatography with a refractive index detector (SEC-RI) plotted as a function of time for solubilized fibroin in 9.3 M LiBr at 122° C.-125° C. (i.e., 3 degree Celsius temperature gradient between 122° C. and 125° C.).

FIG. 11 is a graph illustrating percentage of amino acid modification in silk.

FIG. 12A-FIG. 12C are graphs illustrating percentage of amino acid modifications in Low Skid Silk and Mid Skid silk. FIG. 12A illustrates heavy chain modifications, FIG. 12B illustrates light chain modifications, and FIG. 12C illustrates fibrohexamerin modifications. N are Asparagines that become aspartic acid and Q are Glutamines that become deamidated. M corresponds to Methionies that become oxidized. The numbers after each amino acid show its position along the amino acid chain from the corresponding protein.

FIG. 13A-FIG. 13B are graphs illustrating percentage of amino acid modifications in Low Skid Silk and Mid Skid silk produced and lyophilized. FIG. 13A illustrates heavy chain modifications and FIG. 13B illustrates light chain modifications. N are Asparagines that become aspartic acid and Q are Glutamines that become deamidated. M corresponds to Methionies that become oxidized. The numbers after each amino acid show its position along the amino acid chain from the corresponding protein.

FIG. 14A-FIG. 14B are graphs illustrating percentage of amino acid modifications in Low Skid silk produced in Walpole and Medford using the Skid process with differing process parameters and variable levels. FIG. 14A illustrates heavy chain modifications and FIG. 14B illustrates light chain modifications. N are Asparagines that become aspartic acid and Q are Glutamines that become deamidated. M corresponds to Methionies that become oxidized. The numbers after each amino acid show its position along the amino acid chain from the corresponding protein.

FIG. 15A-FIG. 15D are graphs illustrating percentage of amino acid modifications in Low and Mid silk produced in Skid and Benchtop processes. N are Asparagines that become aspartic acid and Q are Glutamines that become deamidated. M corresponds to Methionies that become oxidized. The numbers after each amino acid show its position along the amino acid chain from the corresponding protein.

FIG. 16 is an explanation of the method used to calculate percentage ratios of modified amino acids at specific locations along the sequence of each peptide.

FIG. 17 illustrates an Anion exchange chromatography and size exclusion chromatography scheme of the isolation of Low Skid silk/modified peptide compositions. Low Skid silk/modified polypeptide compositions is composed of a variety of peptide populations, in a wide range of sizes and charge. Using Q-Sepharose anion exchange chromatography as a first step, and HiLoad Superdex 200 size exclusion chromatography as a second purification step, distinct populations of Low Skid silk/modified polypeptide compositions were separated. The Q-Sepharose eluate was loaded onto HiLoad Superdex 200 size exclusion chromatography, which resulted in negatively charged silk compositions/modified peptides fractionated by size.

FIG. 18A and FIG. 18B are chromatograms of the anion exchange chromatography and the following size exclusion chromatography of the eluate (Q-eluate) of Low Skid silk/modified polypeptide compositions. FIG. 18A: Anion exchange chromatography was performed with a Q-Sepharose column (Cytiva). Low Skid silk/modified peptide compositions were separated to uncharged peptide population (flowthrough—light blue background) and eluted negatively charged silk compositions (eluate—light pink background) by anion exchange chromatography. Light yellow background indicates column wash with 50 mM Tris pH=8.0 before eluting the charged peptide population. FIG. 18B: The negatively charged eluate was loaded onto the Superdex 200 column and was flowed through the column with 50 mM Tris, 200 mM CaCl₂), pH=8.0. When the UV-280 absorbance started to increase fractions were collected to separate the Low Skid silk/modified peptide compositions by size. The relative elution volume of silk compositions AS77 and AS81 are indicated on the chromatogram.

FIG. 19A and FIG. 19B illustrates the Analytical Size Exclusion Chromatography of Low Skid silk/modified silk compositions and their constituent AS compositions. FIG. 19A. Average molecular weight in kDa of Low Skid silk (LS) and AS77-AS81 are shown. FIG. 19B. Polydispersity (PDI) measurements are shown. The numerical data is presented in Table 7.

FIG. 20 is a SDS polyacrylamide gel electrophoresis of Low Skid silk/modified polypeptide compositions. Lanes are indicated by fraction number, at the order of elution from the Superdex 200 column, and their respective silk composition: fraction 6 is AS77, fraction 7 is AS78, fraction 8 is AS79, fraction 9 is AS80, and fraction 10 is AS81.

FIG. 21A and FIG. 21B are graphs illustrating self-assembly reactions of the of the Low Skid silk/modified peptide compositions. Mid Skid Silk reaction was used as a positive control. FIG. 21A. Illustrate kinetic parameters of gel formation during self-assembly of silk. Self-Assembly parameters of Mid Skid silk: Amax is 0.6780 (Abs), SARF is 8.676, T0.5 is 3.668 h, and the FSAF is 3.08 (Abs/min). FIG. 21B. Is a snapshot of a later time point of the same self-assembly assay, 12 days after setting the assay. None of the tested fraction has self-assembled over time.

FIG. 22A and FIG. 22B illustrate the characterization of Low Skid silk compositions by Dynamic Light Scattering. Low skid silk/modified peptide compositions were diluted to a concentration of 1 mg/mL, filtered, and analyzed by the Zetasizer Pro to estimate the diameter particle size of each silk composition. FIG. 22A. Illustrates intensity diameter particle size distribution measured for silk compositions AS77, AS78, AS79, AS80, and AS81. FIG. 22B. Illustrate correlogram functions of silk compositions AS77, AS78, AS79, AS80, AS81.

FIG. 23 illustrates size exclusion chromatography scheme of the isolation of Low Skid silk/modified peptide compositions. Low Skid silk/modified polypeptide compositions is composed of a variety of peptide populations, in a wide range of sizes, using HiLoad Superdex 200 size exclusion chromatography, distinct populations of Low Skid silk/modified polypeptide compositions were separated.

FIG. 24 is a chromatogram of Low Skid silk/modified polypeptide compositions loaded onto a Superdex 200 gel filtration column. Low Skid silk/modified peptide compositions were loaded onto the Superdex 200 column and were flowed through the column with 50 mM Tris, 200 mM CaCl₂), pH=8.0. When the UV-280 absorbance started to increase fractions were collected to separate the Low Skid silk/modified peptide compositions by size. The relative elution volume of silk compositions AS82, AS86, and AS87 are indicated on the chromatogram.

FIG. 25A and FIG. 25B illustrate Analytical Size Exclusion Chromatography of Low Skid silk/modified silk compositions and their constituent AS compositions. FIG. 25A. Illustrates average molecular weight in kDa of Low Skid silk (LS) and AS82-AS89 are shown. FIG. 25B. Polydispersity (PDI) measurements are shown. The numerical data is presented in Table 9.

FIG. 26 is an SDS polyacrylamide gel electrophoresis of Low Skid silk/modified polypeptide compositions. Lanes are indicated by fraction number, at the order of elution from the Superdex 200 column, and their respective silk composition: fraction 6 is AS82, fraction 7 is AS83, fraction 8 is AS84, fraction 9 is AS85, and fraction 10 is AS86.

FIG. 27A and FIG. 27B are graphs illustrating self-assembly reactions of the of the Low Skid silk/modified peptide compositions. Mid Skid Silk reaction was used as a positive control. FIG. 27A. Illustrates kinetic parameters of gel formation during self-assembly of silk. Self-Assembly parameters of Mid Skid silk: Amax is 0.6978 (Abs), SARF is 8.591, T0.5 is 3.361 h, and the FSAF is 3.46 (Abs/min). FIG. 27B. Is a snapshot of a later time point of the same self-assembly assay, 18 days after setting the assay. AS87, AS88, and AS89 demonstrate gel formation at this time point, that was already observed five days post assay (LS, Low Skid silk; MS, Mid Skid silk).

FIG. 28A-FIG. 28C are graphs showing characterization of Low Skid silk compositions by Dynamic Light Scattering. Low skid silk/modified peptide compositions were diluted to a concentration of 1 mg/mL, filtered, and analyzed by the Zetasizer Pro to estimate particle size of each silk composition. FIG. 28A. Shows intensity particle size distribution measured for silk compositions AS82, AS83, AS84, AS85, AS86, AS87, AS88, and AS89. FIG. 28B. Shows intensity particle size distribution measured for silk compositions AS82, Low Skid silk/modified peptide compositions (LS), and Mid Skid silk/modified peptide compositions (MS). FIG. 28C. Shows correlogram functions of silk compositions AS82, AS83, AS84, AS85, AS86, AS87, AS88, AS89, Low Skid silk/modified peptide compositions (LS), Mid Skid silk/modified peptide compositions (MS).

FIG. 29 illustrates anion exchange chromatography (Q), hydrophobic interaction chromatography (HIC), and size exclusion chromatography (SEC) scheme of the isolation of Low Skid silk/modified peptide compositions. Low Skid silk/modified polypeptide compositions is composed of a variety of peptide populations, in a wide range of sizes and charge. Using Q-Sepharose anion exchange chromatography as a first step, Butyl ImpRes Hydrophobic interactions resin as a second step, and HiLoad Superdex 200 size exclusion chromatography as a third purification step, distinct populations of Low Skid silk/modified polypeptide compositions were isolated. The Q-Sepharose eluate was loaded onto a Butyl ImpRes (HIC) column, and the HIC-eluate was loaded onto a HiLoad Superdex 200 size exclusion chromatography, which resulted in fractionation of negatively charged silk compositions/modified peptides with hydrophobicity characteristics fractionated by size. The Q-Sepharose eluate contained negatively charged peptides in all sizes. Resolving these peptides by Butyl ImpRes column resulted in elution of high-molecular-weight, negatively charged, somewhat hydrophobic silk compositions/modified peptides. The smaller negatively charged peptides were washed as flowthrough and did not bind the Butyl ImpRes column. The Q-HIC(elution) was loaded into Superdex 200 and was separated by size.

FIG. 30A-FIG. 30E are chromatograms of anion exchange chromatography, hydrophobic interactions chromatography, and the following size exclusion chromatography of Low Skid silk/modified polypeptide compositions. FIG. 30A. illustrates anion exchange chromatography was performed with a Q-Sepharose column. Low Skid silk/modified peptide compositions were separated to uncharged peptide population (flowthrough—light blue background) and eluted negatively charged silk compositions (eluate—light pink background) by anion exchange chromatography. Light yellow background indicates column wash with 50 mM Tris pH=8.0 before eluting the charged peptide population. FIG. 30B. illustrates the negatively charged eluate (Q-elution) was loaded onto a Butyl ImpRes column, in the presence of 300 mM ammonium sulfate [(NH4)2SO4], to expose hydrophobic domains of the silk peptides, which allows binding to the column. The highly charged peptide population did not bind the column (flowthrough), highlighted in light blue. The column was washed until OD280 was reduced to ˜100 units (light yellow). Then, the bound silk peptides (Q-HIC(elution)) were eluted by using 50 mM Tris, pH=8.0 without ammonium sulfate (light pink). FIG. 30C. illustrates the Q-HIC(elution) was further fractionated by size exclusion chromatography (SEC), using the gel filtration column Superdex 200. The Q-HIC(elution) fraction was flowed through the column with 50 mM Tris, 200 mM CaCl₂), pH=8.0. When the UV-280 absorbance started to increase fractions were collected to separate the Low Skid silk/modified peptide compositions by size. The relative elution volume of silk compositions AS90 and AS94 are indicated on the chromatogram. FIG. 30D. the Q-HIC(flowthrough) fraction was further fractionated by SEC, using the Superdex 200 column, at the same procedure as in (VC). The relative elution volume of silk compositions AS95 and AS100 are indicated on the chromatogram. FIG. 30E. illustrates the superimposition of chromatograms (VC) and (VD). the Q-HIC(elution) fraction has a higher-molecular-weight range compared to the Q-HIC(flowthrough) fractions, which elutes later in SEC, and has lower-molecular-weight range.

FIG. 31A-FIG. 31B are graphs showing analytical Size Exclusion Chromatography of Low Skid silk/modified silk compositions and their constituent AS compositions. FIG. 31A. Average molecular weight in kDa of Low Skid silk (LS) and AS90-AS100 are shown. FIG. 31B. Polydispersity (PDI) measurements are shown. The numerical data is presented in Table 11.

FIG. 32A-FIG. 32B are SDS polyacrylamide gel electrophoresis of Low Skid silk/modified polypeptide compositions. FIG. 32A. Q-HIC(elution) SEC fractions. FIG. 32B. Q-HIC(flowthrough) SEC fractions. Lanes are indicated by fraction number, at the order of elution from the Superdex 200 column, and their respective silk composition: in FIG. 32A, fraction 6 is AS90, fraction 7 is AS91, fraction 8 is AS92, fraction 9 is AS93, and fraction 10 is AS94. In FIG. 32B, fraction 8 is AS95, fraction 9 is AS96, fraction 10 is AS97, fraction 11 is AS98, fraction 12 is AS99, and fraction 13 is AS100.

FIG. 33 illustrates self-assembly reactions of the of the Low Skid silk/modified peptide compositions. Mid Skid Silk reaction was used as a positive control. kinetic parameters of gel formation during self-assembly of silk. Q-HIC(elution) is the elution fraction that was eluted from the Butyl ImpRes column, prior to SEC purification; LS, Low Skid silk; MS, Mid Skid silk. Self-Assembly parameters of Mid Skid silk: Amax is 0.6974 (Abs), SARF is 8.661, T0.5 is 3.834 h, and the FSAF is 3.03 (Abs/min).

FIG. 34A-FIG. 34F illustrate the characterization of Low Skid silk compositions by Dynamic Light Scattering. Low and Mid skid silk/modified peptide compositions were diluted to a concentration of 1 mg/mL, filtered, and analyzed by the Zetasizer Pro to estimate the diameter particle size of each silk composition. FIG. 34A. Intensity diameter particle size distribution measured for silk compositions AS90, Q-HIC(elution) fraction (prior to fractionation by SEC), Low Skid silk (LS), and Mid Skid silk (MS). FIG. 34B. Correlogram functions of silk compositions presented in (34A). FIG. 34C. Intensity diameter particle size distribution measured for silk compositions AS90-AS94, derived from Q-HIC(elution)-SEC fractionation process. FIG. 34D. Correlogram functions of silk compositions presented in (34C). FIG. 34E. Intensity diameter particle size distribution measured for silk compositions AS95-AS100, derived from Q-HIC(flowthrough)-SEC fractionation process. FIG. 34F. Correlogram functions of silk compositions presented in (34E).

FIG. 35 illustrates size exclusion chromatography scheme of the isolation of Mid Skid silk/modified peptide compositions. Mid Skid silk/modified polypeptide compositions is composed of a variety of peptide populations, in a wide range of sizes, using HiLoad Superdex 200 size exclusion chromatography, distinct populations of Mid Skid silk/modified polypeptide compositions were able to be separated.

FIG. 36 is a chromatogram of Mid Skid silk/modified polypeptide compositions loaded onto a Superdex 200 gel filtration column. Mid Skid silk/modified peptide compositions were loaded onto the Superdex 200 column and were flowed through the column with 50 mM Tris, 200 mM CaCl₂), pH=8.0. When the UV-280 absorbance started to increase fractions were collected to separate the Mid Skid silk/modified peptide compositions by size. The relative elution volume of silk compositions AS107 and AS111 are indicated on the chromatogram.

FIG. 37A-FIG. 37B. illustrate analytical Size Exclusion Chromatography of Mid Skid silk/modified silk compositions and their constituent AS compositions. FIG. 37A. Average molecular weight in kDa of Mid Skid silk (MS) and AS106-AS111 are shown. FIG. 37B. Polydispersity (PDI) measurements are shown. The numerical data is presented in Table 14.

FIG. 38 is a SDS polyacrylamide gel electrophoresis of Mid Skid silk/modified polypeptide compositions. Lanes are indicated by fraction number, at the order of elution from the Superdex 200 column, and their respective silk composition: fraction 6 is AS107, fraction 7 is AS108, fraction 8 is AS109, fraction 9 is AS110, and fraction 10 is AS111.

FIG. 39 illustrates self-assembly reactions of the of the Mid Skid silk/modified peptide compositions. Kinetic parameters of gel formation during self-assembly of silk. Dashed red lines show how the self-assembly parameters Amax, SARF, and T0.5 were calculated for unfractionated Mid Skid silk (MS). These numerical calculated parameters of silk compositions AS106-AS111 can be found in Table 16. Low Skid silk (LS) was used as a negative control. LS, Low Skid silk; MS, Mid Skid silk.

FIG. 40A-FIG. 40B. illustrate characterization of Mid Skid silk compositions by Dynamic Light Scattering. Mid skid silk/modified peptide compositions were diluted to a concentration of 1 mg/mL, filtered, and analyzed by the Zetasizer Pro to estimate particle size of each silk composition. FIG. 40A. Intensity particle size distribution measured for silk compositions AS106, AS107, AS108, AS109, AS110, AS111, and Mid Skid (MS). FIG. 40B. Correlation functions of silk compositions presented in (40A).

FIG. 41 illustrates anion exchange chromatography and size exclusion chromatography scheme of the isolation of Mid Skid silk/modified peptide compositions. Mid Skid silk/modified polypeptide compositions is composed of a variety of peptide populations, in a wide range of sizes and charge. Using Q-Sepharose anion exchange chromatography as a first step, and HiLoad Superdex 200 size exclusion chromatography as a second purification step, distinct populations of Mid Skid silk/modified polypeptide compositions were separated. The Q-Sepharose eluate was loaded onto HiLoad Superdex 200 size exclusion chromatography, which resulted in negatively charged silk compositions/modified peptides fractionated by size.

FIG. 42A-FIG. 42B are chromatograms of the anion exchange chromatography and the following size exclusion chromatography of the eluate (Q-eluate) of Mid Skid silk/modified polypeptide compositions. FIG. 42A. Anion exchange chromatography was performed with a Q-Sepharose column (Cytiva). Mid Skid silk/modified peptide compositions were separated to uncharged peptide population (flowthrough—light blue background) and eluted negatively charged silk compositions (eluate—light pink background) by anion exchange chromatography. Light yellow background indicates column wash with 50 mM Tris pH=8.0 before eluting the charged peptide population. FIG. 42B. The negatively charged eluate (Q-elution) was loaded onto the Superdex 200 column and was flowed through the column with 50 mM Tris, 200 mM CaCl₂), pH=8.0. When the UV-280 absorbance started to increase fractions were collected to separate the Mid Skid silk/modified peptide compositions by size. The relative elution volume of silk compositions AS101 and AS105 are indicated on the chromatogram.

FIG. 43A-FIG. 43B illustrate analytical Size Exclusion Chromatography of Mid Skid silk/modified silk compositions and their constituent AS compositions. FIG. 43A. Average molecular weight in kDa of Mid Skid silk (MS) and AS101-AS105 are shown. FIG. 43B. Polydispersity (PDI) measurements are shown. The numerical data is presented in Table 16.

FIG. 44A is a SDS polyacrylamide gel electrophoresis of Mid Skid silk/modified polypeptide compositions. Lanes are indicated by fraction number, at the order of elution from the Superdex 200 column, and their respective silk composition: fraction 6 is AS101, fraction 7 is AS102, fraction 8 is AS103, fraction 9 is AS104, and fraction 10 is AS105.

FIG. 44B illustrates self-assembly reactions of the of the Mid Skid silk/modified peptide compositions. Low Skid Silk reaction was used as a negative control. Kinetic parameters of gel formation during self-assembly of silk are shown. Red dotted lines are shown to clarify the calculations of Amax, SARF (Self-Assembly Rate Factor), and T0.5 parameters in Table 17.

FIG. 45A-FIG. 45C are graphs illustrating characterization of Mid Skid silk compositions by Dynamic Light Scattering. Mid skid silk/modified peptide compositions were diluted to a concentration of 1 mg/mL, filtered, and analyzed by the Zetasizer Pro (Malvern) to estimate the diameter particle size of each silk composition. FIG. 45A. Intensity diameter particle size distribution by intensity measured for silk compositions AS101, AS102, AS103, AS104, and AS105. FIG. 45B. Intensity diameter particle size distribution by intensity measured for silk compositions AS101, AS105, and Mid Skid silk (MS), to emphasize the size difference between AS101 and AS105. FIG. 45C. Correlogram functions of silk compositions AS101, AS102, AS103, AS104, AS105, and Mid Skid silk (MS).

FIG. 46 is an illustration of the values for three molar mass moments (Mn, Mw, and Mz) as it relates to molar mass and the number of molecules at each molar mass. This example is applicable to a polydisperse sample; for a monodisperse sample, Mn=Mw=Mz.

FIG. 47A-FIG. 47B are analytical SEC-MALS of Low, Mid and High Molecular Weight Silk. FIG. 47A. Weight Average Molecular Weight in kDa of Low, Mid, and High Molecular Weight Silk. FIG. 47B. Polydispersity Index (PDI) measurements of Low, Mid, and High Molecular Weight Silk are shown.

FIG. 48A-FIG. 48B are analytical SEC-MALS of Low, Mid and High Molecular Weight Silk organized by Silk Type produced by different process parameters and variable levels. Individual data points are shown and the mean is represented by the height of the box. The bars encompass one standard deviation. FIG. 48A. Weight-Average Molecular Weight Ranges for Low, Mid, and High Molecular Weight Silk. FIG. 48B. PDI Ranges for Low, Mid, and High Molecular Weight Silk.

FIG. 49A-FIG. 49B are analytical SEC-MALS of Low Skid silk/modified silk compositions and the constituent AS compositions as separated by Q-SEC (Q-eluent). FIG. 49A. Average molecular weight in kDa of Low Skid silk (LS) and AS77-AS81 are shown. FIG. 49B. Polydispersity (PDI) measurements are shown. The numerical data is presented in Table 24.

FIG. 50A-FIG. 50B are analytical SEC-MALS of Low Skid silk/modified silk compositions and the constituent AS compositions as separated by SEC. FIG. 50A. Average molecular weight in kDa of Low Skid silk (LS) and AS82-AS89 are shown. FIG. 50B. Polydispersity (PDI) measurements are shown. The numerical data is presented in Table 25.

FIG. 51A-FIG. 51B are analytical SEC-MALS of Low Skid silk/modified silk compositions and the constituent AS compositions as separated by Q-HIC-SEC (Q-HIC-Eluent). FIG. 51A. Average molecular weight in kDa of Low Skid silk (LS) and AS90-AS94 are shown. FIG. 51B. Polydispersity (PDI) measurements are shown. The numerical data is presented in Table 26.

FIG. 52A-FIG. 52B are analytical SEC-MALS of Low Skid silk/modified silk compositions and the constituent AS compositions as separated by Q-HIC-SEC (Q-HIC-Flowthrough). FIG. 52A. Average molecular weight in kDa of Low Skid silk (LS) and AS95-AS100 are shown. FIG. 52B. Polydispersity (PDI) measurements are shown. The numerical data is presented in Table 26.

FIG. 53A-FIG. 53B are analytical SEC-MALS of Mid Skid silk/modified silk compositions and the constituent AS compositions as separated by Q-SEC (Q-flow through). FIG. 53A. Average molecular weight in kDa of Mid Skid silk (MS) and AS101-AS105 are shown. FIG. 53B. Polydispersity (PDI) measurements are shown. The numerical data is presented in Table 27.

FIG. 54A-FIG. 54B are analytical SEC-MALS of Mid Skid silk/modified silk compositions and the constituent AS compositions as separated by SEC. FIG. 54A. Average molecular weight in kDa of Mid Skid silk (MS) and AS106-AS111 are shown. FIG. 54B. Polydispersity (PDI) measurements are shown. The numerical data is presented in Table 28.

FIG. 55 is a chart illustrating mean TEWL values for barrier redux emulsion.

FIG. 56 is a chart illustrating the effect of the barrier redux emulsion on wrinkles and photoaging.

FIG. 57 is a chart illustrating the effect of the barrier redux emulsion on hyperpigmentation.

FIG. 58 is a chart illustrating the effect of the barrier redux emulsion on wrinkles.

FIG. 59 is a chart illustrating the effect of the barrier redux emulsion on crow's feet wrinkles.

FIG. 60 is a chart illustrating the effect of the barrier redux emulsion on crow's feet lines.

FIG. 61 is a chart illustrating effect of the barrier redux emulsion on marionette area wrinkles.

FIG. 62 is a chart illustrating the effect of the barrier redux emulsion on skin roughness.

FIG. 63 is a chart illustrating the effect of the barrier redux emulsion on global wrinkles.

FIG. 64 is a chart illustrating the effect of the barrier redux emulsion on global fine lines.

FIG. 65 is a chart illustrating the effect of the barrier redux emulsion on the appearance of medium, deep lines and wrinkles.

FIG. 66 is a chart illustrating the effect of the barrier redux emulsion on skin tone evenness.

FIG. 67 is a chart illustrating the effect of the barrier redux emulsion on skin texture.

FIG. 68 is a chart illustrating the effect of the barrier redux emulsion on skin scaling.

FIG. 69A-FIG. 69C show the sequence listing for fibroin heavy chain.

FIG. 70 shows the sequence listing for fibroin light chain.

FIG. 71 shows the sequence listing for fibrohexamerin.

FIG. 72 is a graph illustrating absorbency of different fabrics using laundry pods with activated silk.

FIG. 73 is a flow chart showing various embodiments for producing silk fibroin protein fragments (SPFs) of the present disclosure.

FIG. 74 is a flow chart showing various parameters that can be modified during the process of producing a silk protein fragment solution of the present disclosure during the extraction and the dissolution steps.

FIG. 75 is a chart showing absorbency of Activated Silk™ with Capryl/Caprylyl glucoside coating on broad range of interlock nylon fabrics; unfinished nylon interlock fabrics are not absorbing water having poor absorbency; after Activated Silk™ with Capryl/Caprylyl glucoside coating, the absorbency of all the nylon interlock fabrics are significantly increased.

FIG. 76 is a chart showing absorbency of Activated Silk™ with Capryl/Caprylyl glucoside coating on various nylon fabrics other than interlock structure; unfinished nylon fabrics are not absorbing water or having poor absorbency; after Activated Silk™ with Capryl/Caprylyl glucoside coating, the absorbency of all the nylon fabrics are significantly increased.

FIG. 77 is a chart showing the moisture absorbency curve with no washing generated by changing the concentration of polyoxyethylene (29) castor oil in the mixture of emulsifiers (thereby changing the HLB) before adding to the coating solution; in all samples the silk concentration in the coating solution 1 g/L.

FIG. 78 is a chart showing the hand feel ranking curve with no washing generated by changing the concentration of polyoxyethylene (29) castor oil in the mixture of emulsifiers (thereby changing the HLB) before adding to the coating solution; in all samples the silk concentration in the coating solution 1 g/L.

FIG. 79A-FIG. 79D are charts showing the moisture management data for no washes (FIG. 79A), 5 washes (FIG. 79B), 10 washes (FIG. 79C), and 25 washes (FIG. 79D) generated by changing the concentration of the emulsion mixture (Polyoxyethylene (20) sorbitan monooleate, Polyoxyethylene (20) sorbitan trioleate, Polyoxyethylene (29) castor oil, and water in a 2:4:8:10 ratio) in the final coating solution; in all samples the silk concentration in the coating solution 1 g/L.

FIG. 80A-FIG. 80D are charts showing the hand feel ranking results from no washes (FIG. 80A), 5 washes (FIG. 80B), 10 washes (FIG. 80C), and 25 washes (FIG. 80D) generated by changing the concentration of the emulsion mixture (Polyoxyethylene (20) sorbitan monooleate, Polyoxyethylene (20) sorbitan trioleate, Polyoxyethylene (29) castor oil, and water in a 2:4:8:10 ratio) in the final coating solution; 1 is the best score and 8 is the worst hand ranking score; in all samples the silk concentration in the solution 1 g/L.

FIG. 81A-FIG. 81D are charts showing the hand feel ranking results from no washes (FIG. 81A), 5 washes (FIG. 81B), 10 washes (FIG. 81C), and 25 washes (FIG. 81D) generated by changing the concentration of mid molecular weight silk in the final coating solution; 1 is the best ranking and 8 is the worst ranking

FIG. 82A-FIG. 82D are charts showing the moisture management results from no washes (FIG. 82A), 5 washes (FIG. 82B), 10 washes (FIG. 82C), and 25 washes (FIG. 82D) generated by changing the concentration of mid molecular weight silk in the final coating solution.

FIG. 83A-FIG. 83D are graphs showing UV/Vis quantification experiments of fabrics coated with low molecular weight activated silk and polyoxyethylene (20) monooleate solution. FIG. 83A: A graph showing the percent of silk lost after five washes with respect to fiber surface area. FIG. 83B: A graph showing the mass quantified of silk on the fabric after coating with respect to fiber surface area. FIG. 83C: A graph showing the percent of silk lost after five washes with respect to fabric type. FIG. 83D: A graph showing the mass of silk quantified on each fabric before and after five washes depending on fabric type.

FIG. 84 is a chart showing UV/Vis quantification experiments of fabrics coated with low molecular weight activated silk and polyoxyethylene (20) monooleate solution. A graph showing the mass quantified of silk on the fabric after coating with respect to fabric mass in grams per square meter (GSM). This mass is dependent on knit type, fiber content, and filament denier.

FIG. 85A-FIG. 85C include a series of charts showing potentiometric titration curves with the charge density measured at a pH of 5 for the unfinished heavy weight double knit nylon fabric (FIG. 85A), activated silk finished heavy weight double knit nylon fabric (FIG. 85B), and Archroma RPU wetting agent finished heavy weight double knit nylon fabric (FIG. 85C). Each fabric has a titration curve obtained at no washes (FIGS. 85A-85C, left panels) and at five washes (FIGS. 85A-85C, right panels). The change in charge density at pH 5 after washing is denoted as AC.

FIG. 86 illustrates experimental setup for Example 18. Samples of fabric were prepared and washed for five washes in a front-loading washer and surface charging was measured as the difference in charge before and after washing.

FIG. 87 is a diagram showing the location and description of the zeta potential of a surface.

FIG. 88 is a graph showing the change in zeta potential before and after five washes for five different fabric types and three different finishing types.

FIG. 89 is a diagram showing the effective surface charge has on surrounding pH due to proton and hydroxide surface adsorption.

FIG. 90 is a graph showing the change in charge density at pH 5 before and after five washes for five different fabric types. The control section denotes the unfinished fabric and the AS 320 section denotes the silk finished fabrics.

FIG. 91 is a graph illustrating the UV/Vis spectrum of synthesized Silver Silk Nanoparticles tested in Example 19.

FIG. 92 is a graph showing absorbency of the unfinished, Nigh Blue DC98-IA pretreated with fixing agent only, and AS-320 coated Night Blue DC98-IA, pretreated with cationic fixing agent from T=0 to T=10.

FIG. 93 is a graph showing absorbency of the unfinished, Black DC95-FA pretreated with fixing agent only, and AS-320 coated Black DC95-FA, pretreated with cationic fixing agent from T=0 to T=10.

FIG. 94 is a graph showing absorbency of unfinished at T=0, and AS-320 coated (15 g/L, with and without HT-232H fixing agent pretreatment) 17120513 black from T=0 to T=10.

FIG. 95 is a graph showing absorbency of unfinished at T=0, and AS-320 coated (15 g/L, with and without HT-232H fixing agent pretreatment) 17120511 blue from T=0 to T=10.

FIG. 96 is a graph illustrating the improvement in Investigator Global Assessment (IGA) after using the Body Wash.

FIG. 97 is a graph illustrating the improvement in redness after using the Body Wash.

FIG. 98 is a graph illustrating the improvement in eczema count after using the Body Wash.

FIG. 99 is a graph illustrating the improvement in individual corneometer after using the Body Wash.

FIG. 100 is a graph illustrating the improvement in individual TEWL after using the Body Wash.

FIG. 101 is a graph illustrating responses of the Self-perception Questionnaires after using the Body Wash.

FIG. 102 is a graph illustrating responses of the Self-perception Questionnaires after using the Body Wash.

FIG. 103 illustrates three chromatography principles of silk fractionalization.

FIG. 104 illustrates the size exclusion chromatography of silk fractionalization.

FIG. 105 illustrates the anion exchange chromatography followed by size exclusion chromatography of silk fractionalization.

FIG. 106 illustrates the anion exchange chromatography followed by hydrophobic interactions chromatography and size exclusion chromatography.

FIG. 107 is a chart summarizing the three pipelines of silk fractionalization.

FIG. 108 is the characterizations methods of silk fractionalization.

FIG. 109 illustrates size exclusion chromatography of Low Skid silk.

FIG. 110 illustrates an example of silk composition characterization: Size exclusion chromatography of Low Skid silk.

FIG. 111 illustrates an example of silk composition characterization: Size exclusion chromatography of Low Skid silk Dynamic light scattering.

FIG. 112 is a chart including Z-averages of Low Skid silk/modified polypeptide composition.

FIG. 113 illustrates molecular weight and Polydispersity determination by HPLC.

FIG. 114 illustrates Silk fractionation Size exclusion chromatography of Low Skid silk. Lower-molecular-weight fractions self-assemble slowly over time.

FIG. 115 is a chart including assays for characterizing silk fractions.

FIG. 116 is a comparison of Molecular weight and Polydispersity determination by two methods.

FIG. 117 shows graphs presenting the data of Tables 70 and 71. Data shown with standard deviation. “Nanoclay” refers to Elementis Bentone Hydroclay.

FIG. 118 is as diagram of the typical bentonite clay structure.

FIG. 119 shows SEM images of cross-sectional area of film cast with Elementis Bentone Hydroclay 2001. Highly-ordered stacking of clay layers is visible.

FIG. 120 is an SEM image of cross-sectional area of film cast with pure RSF.

FIG. 121 is an SEM image of cross-sectional area of 1:1 RSF/2001 film cast under neutral (pH ˜7.0) conditions. The layered structure of the clay is retained.

FIG. 122 is an SEM image of cross-sectional area of 1:1 RSF/2001 film cast under acidic (pH ˜3.5) conditions. Note the ribbon-like structure.

FIG. 123 illustrates the increased diffusive pathway created by the RSF/nanoclay composite.

FIG. 124 is an FTIR scan of the amide I region of RSF/2001 films cast under neutral conditions. The concentration of nanoclay is varied from 0% (red) to 70% (yellow). As nanoclay content increases, the amide I peak shifts left, away from the beta sheet region.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

In some embodiments, compositions of the present disclosure include peptides compositions selected from compositions #1001 to #2450, having weight average molecular weights selected from about 1 kDa to about 145 kDa, and a polydispersity selected from between 1 and about 5 (including, without limitation, a polydispersity of 1), between 1 and about 1.5 (including, without limitation, a polydispersity of 1), between about 1.5 and about 2, between about 1.5 and about 3, between about 2 and about 2.5, between about 2.5 and about 3, between about 3 and about 3.5, between about 3.5 and about 4, between about 4 and about 4.5, and between about 4.5 and about 5.

Methods of making silk fibroin or silk fibroin protein fragments and their applications in various fields are known and are described for example in U.S. Pat. Nos. 9,187,538, 9,511,012, 9,517,191, 9,522,107, 9,522,108, 9,545,369, and 10,166,177, 10,287,728 and 10,301,768, all of which are incorporated herein in their entireties. Raw silk from silkworm Bombyx mori is composed of two primary proteins: silk fibroin (approximately 75%) and sericin (approximately 25%). Silk fibroin is a fibrous protein with a semi-crystalline structure that provides stiffness and strength. As used herein, the term “silk fibroin” means the fibers of the cocoon of Bombyx mori having a weight average molecular weight of about 370,000 Da. The crude silkworm fiber consists of a double thread of fibroin. The adhesive substance holding these double fibers together is sericin. The silk fibroin is composed of a heavy chain having a weight average molecular weight of about 350,000 Da (H chain), and a light chain having a weight average molecular weight about 25,000 Da (L chain). Silk fibroin is an amphiphilic polymer with large hydrophobic domains occupying the major component of the polymer, which has a high molecular weight. The hydrophobic regions are interrupted by small hydrophilic spacers, and the N- and C-termini of the chains are also highly hydrophilic. The hydrophobic domains of the H-chain contain a repetitive hexapeptide sequence of Gly-Ala-Gly-Ala-Gly-Ser and repeats of Gly-Ala/Ser/Tyr dipeptides, which can form stable anti-parallel-sheet crystallites. The amino acid sequence of the L-chain is non-repetitive, so the L-chain is more hydrophilic and relatively elastic. The hydrophilic (Tyr, Ser) and hydrophobic (Gly, Ala) chain segments in silk fibroin molecules are arranged alternatively such that allows self-assembling of silk fibroin molecules.

As used herein, the term “fibroin” includes silk worm fibroin and insect or spider silk protein. In an embodiment, fibroin is obtained from Bombyx mori. Raw silk from Bombyx mori is composed of two primary proteins: silk fibroin (approximately 75%) and sericin (approximately 25%). As used herein, the term “silk fibroin” means the fibers of the cocoon of Bombyx mori having a weight average molecular weight of about 370,000 Da. Methods of making silk fibroin protein fragments, and/or compositions thereof, are known and are described for example in U.S. Pat. Nos. 9,187,538, 9,511,012, 9,517,191, 9,522,107, 9,522,108, 9,545,369, and 10,166,177.

Recombinant silk described in patents and patent applications, incorporated by reference herein: US 2004590196, U.S. Pat. No. 7,754,851, US 2007654470, U.S. Pat. No. 7,951,908, US 2010785960, U.S. Pat. No. 8,034,897, US 20090263430, US 2008226854, US 20090123967, US 2005712095, US 2007991037, US 20090162896, US 200885266, U.S. Pat. No. 8,372,436, US 2007989907, US 2009267596, US 2010319542, US 2009265344, US 2012684607, US 2004583227, U.S. Pat. No. 8,030,024, US 2006643569, U.S. Pat. No. 7,868,146, US 2007991916, U.S. Pat. No. 8,097,583, US 2006643200, U.S. Pat. Nos. 8,729,238, 8,877,903, US 20190062557, US 20160280960, US 20110201783, US 2008991916, US 2011986662, US 2012697729, US 20150328363, U.S. Pat. No. 9,034,816, US 20130172478, U.S. Pat. No. 9,217,017, US 20170202995, U.S. Pat. No. 8,721,991, US 2008227498, U.S. Pat. Nos. 9,233,067, 8,288,512, US 2008161364, U.S. Pat. No. 7,148,039, U.S. Ser. No. 19/992,47806, US 2001861597, US 2004887100, U.S. Pat. Nos. 9,481,719, 8,765,688, US 200880705, US 2010809102, U.S. Pat. No. 8,367,803, US 2010664902, U.S. Pat. No. 7,569,660, U.S. Ser. No. 19/991,38833, US 2000591632, US 20120065126, US 20100278882, US 2008161352, US 20100015070, US 2009513709, US 20090194317, US 2004559286, US 200589551, US 2008187824, US 20050266242, US 20050227322, and US 20044418.

Recombinant silk is also described in other patents and patent applications, incorporated by reference herein: US 20190062557, US 20150284565, US 20130225476, US 20130172478, US 20130136779, US 20130109762, US 20120252294, US 20110230911, US 20110201783, US 20100298877, U.S. Pat. Nos. 10,478,520, 10,253,213, 10,072,152, 9,233,067, 9,217,017, 9,034,816, 8,877,903, 8,729,238, 8,721,991, 8,097,583, 8,034,897, 8,030,024, 7,951,908, 7,868,146, and 7,754,851.

Recombinant silk protein and/or methods described herein, may include one or more recombinant silk proteins described above or recited in U.S. Pat. Nos. 8,173,772, 8,278,416, 8,618,255, 8,642,734, 8,691,581, 8,729,235, 9,115,204, 9,157,070, 9,309,299, 9,644,012, 9,708,376, 9,051,453, 9,617,315, 9,968,682, 9,689,089, 9,732,125, 9,856,308, 9,926,348, 10,065,997, 10,316,069, and 10,329,332; and U.S. Patent Publication Nos. 2009/0226969, 2011/0281273, 2012/0041177, 2013/0065278, 2013/0115698, 2013/0316376, 2014/0058066, 2014/0079674, 2014/0245923, 2015/0087046, 2015/0119554, 2015/0141618, 2015/0291673, 2015/0291674, 2015/0239587, 2015/0344542, 2015/0361144, 2015/0374833, 2015/0376247, 2016/0024464, 2017/0066804, 2017/0066805, 2015/0293076, 2016/0222174, 2017/0283474, 2017/0088675, 2019/0135880, 2015/0329587, 2019/0040109, 2019/0135881, 2019/0177363, 2019/0225646, 2019/0233481, 2019/0031842, 2018/0355120, 2019/0186050, 2019/0002644, 2020/0031887, 2018/0273590, 20191/094403, 2019/0031843, 2018/0251501, 2017/0066805, 2018/0127553, 2019/0329526, 2020/0031886, 2018/0080147, 2019/0352349, 2020/0043085, 2019/0144819, 2019/0228449, 2019/0340666, 2020/0000091, 2019/0194710, 2019/0151505, 2018/0265555, 2019/0352330, 2019/0248847, and 2019/0378191, the entirety of which are incorporated herein by reference.

As used herein, the term “substantially free of inorganic residuals” means that the composition exhibits residuals of 0.1% (w/w) or less. In an embodiment, substantially free of inorganic residuals refers to a composition that exhibits residuals of 0.05% (w/w) or less. In an embodiment, substantially free of inorganic residuals refers to a composition that exhibits residuals of 0.01% (w/w) or less. In an embodiment, the amount of inorganic residuals is between 0 ppm (“non-detectable” or “ND”) and 1000 ppm. In an embodiment, the amount of inorganic residuals is ND to about 500 ppm. In an embodiment, the amount of inorganic residuals is ND to about 400 ppm. In an embodiment, the amount of inorganic residuals is ND to about 300 ppm. In an embodiment, the amount of inorganic residuals is ND to about 200 ppm. In an embodiment, the amount of inorganic residuals is ND to about 100 ppm. In an embodiment, the amount of inorganic residuals is between 10 ppm and 1000 ppm.

As used herein, the term “substantially free of organic residuals” means that the composition exhibits residuals of 0.1% (w/w) or less, in an embodiment, substantially free of organic residuals refers to a composition that exhibits residuals of 0.05% (w/w) or less. In an embodiment, substantially free of organic residuals refers to a composition that exhibits residuals of 0.01% (w/w) or less. In an embodiment, the amount of organic residuals is between 0 ppm (“non-detectable” or “ND”) and 1000 ppm. In an embodiment, the amount of organic residuals is ND to about 500 ppm. In an embodiment, the amount of organic residuals is ND to about 400 ppm. In an embodiment, the amount of organic residuals is ND to about 300 ppm. In an embodiment, the amount of organic residuals is ND to about 200 ppm. In an embodiment, the amount of organic residuals is ND to about 100 ppm. In an embodiment, the amount of organic residuals is between 10 ppm and 1000 ppm.

Compositions of the present disclosure exhibit “biocompatibility” meaning that the compositions are compatible with living tissue or a living system by not being toxic, injurious, or physiologically reactive and not causing immunological rejection. Such biocompatibility can be evidenced by participants topically applying compositions of the present disclosure on their skin for an extended period of time. In an embodiment, the extended period of time is about 3 days. In an embodiment, the extended period of time is about 7 days, in an embodiment, the extended period of time is about 14 days, in an embodiment, the extended period of time is about 21 days. In an embodiment, the extended period of time is about 30 days. In an embodiment, the extended period of time is selected from the group consisting of about I month, about 2 months, about 3 months, about 4 months, about 5 months, about 6 months, about 7 months, about 8 months, about 9 months, about 10 months, about 11 months, about 12 months, and indefinitely.

Compositions of the present disclosure can be “hypoallergenic” meaning that they are relatively unlikely to cause an allergic reaction. Such hypoallergenicity can be evidenced by participants topically applying compositions of the present disclosure on their skin for an extended period of time. In an embodiment, the extended period of time is about 3 days. In an embodiment, the extended period of time is about 7 days. In an embodiment, the extended period of time is about 14 days. In an embodiment, the extended period of time is about 21 days. In an embodiment, the extended period of time is about 30 days. In an embodiment, the extended period of time is selected from the group consisting of about 1 month, about 2 months, about 3 months, about 4 months, about 5 months, about 6 months, about 7 months, about 8 months, about 9 months, about 10 months, about 11 months, about 12 months, and indefinitely.

In an embodiment, a peptide composition of the present disclosure has non-detectable levels of LiBr residuals. In an embodiment, the amount of the LiBr residuals in a composition of the present disclosure is between 10 ppm and 1000 ppm. In an embodiment, the amount of the LiBr residuals in a composition of the present disclosure is between 10 ppm and 300 ppm. In an embodiment, the amount of the LiBr residuals in a composition of the present disclosure is less than 25 ppm. In an embodiment, the amount of the Li Br residuals in a composition of the present disclosure is less than 50 ppm. In an embodiment, the amount of the LiBr residuals in a composition of the present disclosure is less than 75 ppm. In an embodiment, the amount of the LiBr residuals in a composition of the present disclosure is less than 100 ppm. In an embodiment, the amount of the LiBr residuals in a composition of the present disclosure is less than 200 ppm. In an embodiment, the amount of the LiBr residuals in a composition of the present disclosure is less than 300 ppm. In an embodiment, the amount of the LiBr residuals in a composition of the present disclosure is less than 400 ppm. In an embodiment, the amount of the LiBr residuals in a composition of the present disclosure is less than 500 ppm. In an embodiment, the amount of the LiBr residuals in a composition of the present disclosure is less than 600 ppm. In an embodiment, the amount of the LiBr residuals in a composition of the present disclosure is less than 700 ppm. In an embodiment, the amount of the LiBr residuals in a composition of the present disclosure is less than 800 ppm. In an embodiment, the amount of the LiBr residuals in a composition of the present disclosure is less than 900 ppm. In an embodiment, the amount of the LiBr residuals in a composition of the present disclosure is less than 1000 ppm. In an embodiment, the amount of the LiBr residuals in a composition of the present disclosure is non-detectable to 500 ppm. In an embodiment, the amount of the LiBr residuals in a composition of the present disclosure is non-detectable to 450 ppm. In an embodiment, the amount of the LiBr residue in a composition of the present disclosure is non-detectable to 400 ppm. In an embodiment, the amount of the LiBr residuals in a composition of the present disclosure is non-detectable to 350 ppm. In an embodiment, the amount of the LiBr residuals in a composition of the present disclosure is non-detectable to 300 ppm. In an embodiment, the amount of the LiBr residuals in a composition of the present disclosure is non-detectable to 250 ppm. In an embodiment, the amount of the LiBr residuals in a composition of the present disclosure is non-detectable to 200 ppm. In an embodiment, the amount of the LiBr residuals in a composition of the present disclosure is non-detectable to 150 ppm. In an embodiment, the amount of the LiBr residuals in a composition of the present disclosure is non-detectable to 100 ppm. In an embodiment, the amount of the LiBr residuals in a composition of the present disclosure is 100 ppm to 200 ppm. In an embodiment, the amount of the LiBr residuals in a composition of the present disclosure is 200 ppm to 300 ppm. In an embodiment, the amount of the LiBr residuals in a composition of the present disclosure is 300 ppm to 400 ppm. In an embodiment, the amount of the LiBr residuals in a composition of the present disclosure is 400 ppm to 500 ppm.

In an embodiment, a peptide composition of the present disclosure has non-detectable levels of Na₂CO₃ residuals. In an embodiment, the amount of the Na₂CO₃ residuals in a composition of the present disclosure is less than 100 ppm. In an embodiment, the amount of the Na₂CO₃ residuals in a composition of the present disclosure is less than 200 ppm. In an embodiment, the amount of the Na₂CO₃ residuals in a composition of the present disclosure is less than 300 ppm. In an embodiment, the amount of the Na₂CO₃ residuals in a composition of the present disclosure is less than 400 ppm. In an embodiment, the amount of the Na₂CO₃ residuals in a composition of the present disclosure is less than 500 ppm. In an embodiment, the amount of the Na₂CO₃ residuals in a composition of the present disclosure is less than 600 ppm. In an embodiment, the amount of the Na₂CO₃ residuals in a composition of the present disclosure is less than 700 ppm. In an embodiment, the amount of the Na₂CO₃ residuals in a composition of the present disclosure is less than 800 ppm. In an embodiment, the amount of the Na₂CO₃ residuals in a composition of the present disclosure is less than 900 ppm. In an embodiment, the amount of the Na₂CO₃ residuals in a composition of the present disclosure is less than 1000 ppm. In an embodiment, the amount of the Na₂CO₃ residuals in a composition of the present disclosure is non-detectable to 500 ppm. In an embodiment, the amount of the Na₂CO₃ residuals in a composition of the present disclosure is non-detectable to 450 ppm. In an embodiment, the amount of the Na₂CO₃ residuals in a composition of the present disclosure is non-detectable to 400 ppm. In an embodiment, the amount of the Na₂CO₃ residuals in a composition of the present disclosure is non-detectable to 350 ppm. In an embodiment, the amount of the Na₂CO₃ residuals in a composition of the present disclosure is non-detectable to 300 ppm. In an embodiment, the amount of the Na₂CO₃ residuals in a composition of the present disclosure is non-detectable to 250 ppm. In an embodiment, the amount of the Na₂CO₃ residuals in a composition of the present disclosure is non-detectable to 200 ppm. In an embodiment, the amount of the Na₂CO₃ residuals in a composition of the present disclosure is non-detectable to 150 ppm. In an embodiment, the amount of the Na₂CO₃ residuals in a composition of the present disclosure is non-detectable to 100 ppm. In an embodiment, the amount of the Na₂CO₃ residuals in a composition of the present disclosure is 100 ppm to 200 ppm. In an embodiment, the amount of the Na₂CO₃ residuals in a composition of the present disclosure is 200 ppm to 300 ppm. In an embodiment, the amount of the Na₂CO₃ residuals in a composition of the present disclosure is 300 ppm to 400 ppm. In an embodiment, the amount of the Na₂CO₃ residuals in a composition of the present disclosure is 400 ppm to 500 ppm.

As used herein when referring to a number or a numerical range, the term “about” means that the stated number or numerical range is included together with numbers or numerical ranges within experimental variability, or within statistical experimental error from the stated number or numerical range, wherein the variation or error is from 0% to 15%, or from 0% to 10%, or from 0% to 5% of the stated number or numerical range.

The silk proteins or fragments thereof, silk solutions or mixtures (e.g., SPF or SFS solutions or mixture), and the like, may be prepared according to the methods described in U.S. Pat. Nos. 9,187,538, 9,522,107, 9,522,108, 9,511,012, 9,517,191, 9,545,369, and 10,166,177, and U.S. Patent Publication Nos. 2016/0222579 and 2016/0281294, and International Patent Publication Nos. WO 2016/090055 and WO 2017/011679, the entirety of which are incorporated herein by reference. Methods of using silk fibroin or silk fibroin fragments in coating applications are known and are described for example in U.S. Pat. Nos. 10,287,728 and 10,301,768.

The disclosure provides articles comprising coated fabrics, wherein the coating comprises a surfactant and/or emulsifier system and silk fibroin fragments, including modified silk fragments disclosed herein, e.g., and without limitation, fibroin peptides and/or protein fragments wherein at least one of the amino acids is modified, substituted, or replaced as disclosed herein and methods of making such articles.

Silk is a natural polymer produced by a variety of insects and spiders. Silk produced by Bombyx mori (silkworm) comprises a filament core protein, silk fibroin, and a glue-like coating consisting of a nonfilamentous protein, sericin. Silk fibroin is a FDA approved, edible, non-toxic, and relative inexpensive silkworm cocoon derived proteins. The structure and content of amino acids in the silk fibroin protein are very similar to the tissue of the human body.

Methods of making silk fibroin protein fragments are known and are described for example in U.S. Pat. Nos. 9,187,538, 9,511,012, 9,517,191, 9,522,107, 9,522,108, 9,545,369, and 10,166,177. Methods of using silk fibroin or silk fibroin fragments in coating applications, including coating applications of animal hair, are known and are described for example in U.S. Patent Application Publications Nos. 20160222579, and 20160281294. Compositions and methods of using silk fibroin or silk fibroin fragments in cosmetic applications are known and are described for example in U.S. Patent Application Publications Nos. 20180280274 and 20180008522, and International Patent Application Publication No. WO 2019005848. All of the publications cited herein are incorporated by reference herein in their entireties.

The recent advancements in silk material technology include the emergence of the top-down and bottom-up engineering of silk cocoon, specifically regenerate cocoon into an aqueous silk solution and to use genetic engineering to produce recombinant silk with molecularly defined composition (Tran et al., A review of the emerging role of silk for the treatment of the eye, Pharm. Res., 2018, vol. 35, pp. 1-16). In recent years, silk fibroin proteins are reported to have found applications in ocular tissue reconstruction, corneal tissue engineering and in ocular surface repair due to their biocompatibility, tunable properties, and transparency. Silk films have been found to support corneal cell growth and to develop stratified epithelial cell sheets equivalent to amniotic membrane substrates (Lawrence et al., Silk film biomaterials for cornea tissue engineering, Biomaterials, 2009; 30(7): 1299-308; Harkin et al., Silk fibroin in ocular tissue reconstruction, Biomaterials, 2011; Chirtla et al., Bombyx mori silk fibroin membranes as potential substrata for epithelial constructs used in the management of ocular surface disorders. Tissue Engineering Pan A, 2008; 14(7): 1203-11.). Silk fibroin protein and hydrolyzed peptide fragments have been shown to inhibit transcription and upstream activation of NF-κB protein subunits and proinflammatory molecules that are classically under the control of NF-κB (Hayden et al., Cell Research, 2011. 21(2): 223-244; Chon et al., International Journal of Molecular Medicine, 2012. 30(5): 1203-1210; Kim et al., J. Neurosurg., 2011. 114(2): 485-90; J. Microbiol. Biotechnol., 2012. 22(4): 494-500).

### Definitions

As used in the preceding sections and throughout the rest of this specification, unless defined otherwise, all technical and scientific terms used herein have the same meaning as is commonly understood by one skilled in the art to which this disclosure belongs. All patents and publications referred to herein are incorporated by reference in their entireties.

All percentages, parts and ratios are based upon the total weight of the eye care compositions of the present disclosure, unless otherwise specified. All such weights as they pertain to listed ingredients are based on the active level and, therefore, do not include solvents or by-products that may be included in commercially available materials, unless otherwise specified. The term “weight percent” may be denoted as “wt. %” or % w/w herein.

As used herein, the term “a”, “an”, or “the” generally is construed to cover both the singular and the plural forms.

The term “about” as used herein, generally refers to a particular numeric value that is within an acceptable error range as determined by one of ordinary skill in the art, which will depend in part on how the numeric value is measured or determined, i.e., the limitations of the measurement system. For example, “about” can mean zero variation, and a range of ±20%, ±10%, or ±5% of a given numeric value.

As used herein, the term “dermatologically acceptable carrier” means a carrier suitable for use in contact with mammalian keratinous tissue without causing any adverse effects such as undue toxicity, incompatibility, instability, allergic response, for example. A dermatologically acceptable carrier may include, without limitations, water, liquid or solid emollients, humectants, solvents, and the like.

As used herein, the term “hydrophilic-lipophilic balance” (HLB) of a surfactant and/or emulsifier is a measure of the degree to which it is hydrophilic or hydrophobic, as determined by calculating values for the different regions of the molecule, as described by Griffin's method HLB=20*Mₕ/M, where Mₕ is the molecular mass of the hydrophilic portion of the surfactant and/or emulsifier, and M is the molecular mass of the entire surfactant and/or emulsifier molecule, giving a result on a scale of 0 to 20. A HLB value of 0 corresponds to a completely lipophilic molecule, and a value of 20 corresponds to a completely hydrophilic molecule. The HLB value can be used to predict the surfactant and/or emulsifier properties of a molecule: HLB <10: Lipid-soluble (water-insoluble), HLB >10: Water-soluble (lipid-insoluble), HLB=1-3: anti-foaming agent, 3-6: W/O (water-in-oil) emulsifier, 7-9: wetting and spreading agent, 8-16: O/W (oil-in-water) emulsifier, 13-16: detergent, 16-18: solubilizer or hydrotrope.

As used herein, “average weight average molecular weight” refers to an average of two or more values of weight average molecular weight of silk fibroin or fragments thereof of the same compositions, the two or more values determined by two or more separate experimental readings.

As used herein, the term polymer “polydispersity (PD)” is generally used as a measure of the broadness of a molecular weight distribution of a polymer, and is defined by the formula polydispersity

$$PD = \frac{Mw}{Mn} .$$

As used herein, the term “substantially homogeneous” may refer to silk fibroin-based protein fragments that are distributed in a normal distribution about an identified molecular weight. As used herein, the term “substantially homogeneous” may refer to an even distribution of a component or an additive, for example, silk fibroin fragments, dermatologically acceptable carrier, etc., throughout a composition of the present disclosure.

As used herein, the terms “silk fibroin peptide,” “silk fibroin protein fragment,” and “silk fibroin fragment” are used interchangeably. Molecular weight or number of amino acids units are defined when molecular size becomes an important parameter.

As used herein, the term “fast-dissolving solid forms” refers to fast-dissolving solid forms including freeze dried forms (cakes, wafers, thin films), and compressed tablets.

As used herein, the terms “peptide” or “protein” refers to a chain of amino acids that are held together by peptide bonds (also called amide bonds). The basic distinguishing factors for proteins and peptides are size and structure. Peptides are smaller than proteins. Traditionally, peptides are defined as molecules that consist of between 2 and 50 amino acids, whereas proteins are made up of 50 or more amino acids. In addition, peptides tend to be less well defined in structure than proteins, which can adopt complex conformations known as secondary, tertiary, and quaternary structures.

As used herein, the term “fibroin” or “silk protein” is a type of structural protein produced by certain spider and insect species that produce silk (See definition provided in WIPO Pearl-WIPO's Multilingual Terminology Portal database). Fibroin may include silkworm fibroin, insect or spider silk protein (e.g., spidroin), recombinant spider protein, silk proteins present in other spider silk types, e.g., tubuliform silk protein (TuSP), flagelliform silk protein, minor ampullate silk proteins, aciniform silk protein, pyriform silk protein, aggregate silk glue), silkworm fibroin produced by genetically modified silkworm, or recombinant silkworm fibroin.

As used herein, the term “silk fibroin” refers to silkworm fibroin, silk fibroin produced by genetically modified silkworm, or recombinant silkworm fibroin (See (1) Narayan Ed., Encyclopedia of Biomedical Engineering, Vol. 2, Elsevier, 2019; (2) Kobayashi et al. Eds, Encyclopedia of Polymeric Nanomaterials, Springer, 2014, https://link.springer.com/referenceworkentry/10.1007%2F978-3-642-36199-9_323-1). In an embodiment, silk fibroin is obtained from Bombyx mori.

The term “solid solution” as used herein, refers to the active agent molecularly dissolved in the solid excipient matrix such as hydrophobic polymers, wherein the active agent is miscible with the polymer matrix excipient.

The term “solid dispersion” as used herein, refers to the active agent dispersed as crystalline or amorphous particles, wherein the active agent is dispersed in an amorphous polymer and is distributed at random between the polymer matrix excipient.

As used herein, the term “substantially homogeneous” may refer to silk fibroin-based protein fragments that are distributed in a normal distribution about an identified molecular weight. As used herein, the term “substantially homogeneous” may also refer to an even distribution of a component or an additive, for example, silk fibroin-based protein fragments, dermatologically acceptable carrier, etc., throughout the silk eye care composition.

As used herein, the term “surface tension” refers to the tendency of fluid surfaces to shrink into the minimum surface area possible. At liquid-air interfaces, surface tension results from the greater attraction of liquid molecules to each other (due to cohesion) than to the molecules in the air (due to adhesion). The net effect is an inward force at its surface that causes the liquid to behave as if its surface were covered with a stretched elastic membrane. Because of the relatively high attraction of water molecules to each other through a web of hydrogen bonds, water has a higher surface tension (72.8 mN/m at 20° C.) than most other liquids.

### SPF Definitions and Properties

As used herein, “silk protein fragments” (SPF) include, without limitation, one or more of: “silk fibroin fragments” as defined herein; “recombinant silk fragments” as defined herein; “spider silk fragments” as defined herein; “silk fibroin-like protein fragments” as defined herein; “chemically modified silk fragments” as defined herein; “sericin or sericin fragments” as defined herein; and/or modified silk fragments disclosed herein, e.g., and without limitation, fibroin peptides and/or protein fragments wherein at least one of the amino acids is modified, substituted, or replaced as disclosed herein. SPF may have any molecular weight values or ranges described herein, and any polydispersity values or ranges described herein.

### SPF Molecular Weight and Polydispersity

In an embodiment, a composition of the present disclosure includes SPF having an average weight average molecular weight selected from between about 1 to about 5 kDa. In an embodiment, a composition of the present disclosure includes SPF having an average weight average molecular weight selected from between about 5 to about 10 kDa. In an embodiment, a composition of the present disclosure includes SPF having an average weight average molecular weight selected from between about 10 to about 15 kDa. In an embodiment, a composition of the present disclosure includes SPF having an average weight average molecular weight selected from between about 15 to about 20 kDa. In an embodiment, a composition of the present disclosure includes SPF having an average weight average molecular weight selected from between about 14 to about 30 kDa. In an embodiment, a composition of the present disclosure includes SPF having an average weight average molecular weight selected from between about 20 to about 25 kDa. In an embodiment, a composition of the present disclosure includes SPF having an average weight average molecular weight selected from between about 25 to about 30 kDa. In an embodiment, a composition of the present disclosure includes SPF having an average weight average molecular weight selected from between about 30 to about 35 kDa. In an embodiment, a composition of the present disclosure includes SPF having an average weight average molecular weight selected from between about 35 to about 40 kDa. In an embodiment, a composition of the present disclosure includes SPF having an average weight average molecular weight selected from between about 39 to about 54 kDa. In an embodiment, a composition of the present disclosure includes SPF having an average weight average molecular weight selected from between about 40 to about 45 kDa. In an embodiment, a composition of the present disclosure includes SPF having an average weight average molecular weight selected from between about 45 to about 50 kDa. In an embodiment, a composition of the present disclosure includes SPF having an average weight average molecular weight selected from between about 50 to about 55 kDa. In an embodiment, a composition of the present disclosure includes SPF having an average weight average molecular weight selected from between about 55 to about 60 kDa. In an embodiment, a composition of the present disclosure includes SPF having an average weight average molecular weight selected from between about 60 to about 65 kDa. In an embodiment, a composition of the present disclosure includes SPF having an average weight average molecular weight selected from between about 65 to about 70 kDa. In an embodiment, a composition of the present disclosure includes SPF having an average weight average molecular weight selected from between about 70 to about 75 kDa. In an embodiment, a composition of the present disclosure includes SPF having an average weight average molecular weight selected from between about 75 to about 80 kDa. In an embodiment, a composition of the present disclosure includes SPF having an average weight average molecular weight selected from between about 80 to about 85 kDa. In an embodiment, a composition of the present disclosure includes SPF having an average weight average molecular weight selected from between about 85 to about 90 kDa. In an embodiment, a composition of the present disclosure includes SPF having an average weight average molecular weight selected from between about 90 to about 95 kDa. In an embodiment, a composition of the present disclosure includes SPF having an average weight average molecular weight selected from between about 95 to about 100 kDa. In an embodiment, a composition of the present disclosure includes SPF having an average weight average molecular weight selected from between about 100 to about 105 kDa. In an embodiment, a composition of the present disclosure includes SPF having an average weight average molecular weight selected from between about 105 to about 110 kDa. In an embodiment, a composition of the present disclosure includes SPF having an average weight average molecular weight selected from between about 110 to about 115 kDa. In an embodiment, a composition of the present disclosure includes SPF having an average weight average molecular weight selected from between about 115 to about 120 kDa. In an embodiment, a composition of the present disclosure includes SPF having an average weight average molecular weight selected from between about 120 to about 125 kDa. In an embodiment, a composition of the present disclosure includes SPF having an average weight average molecular weight selected from between about 125 to about 130 kDa. In an embodiment, a composition of the present disclosure includes SPF having an average weight average molecular weight selected from between about 130 to about 135 kDa. In an embodiment, a composition of the present disclosure includes SPF having an average weight average molecular weight selected from between about 135 to about 140 kDa. In an embodiment, a composition of the present disclosure includes SPF having an average weight average molecular weight selected from between about 140 to about 145 kDa. In an embodiment, a composition of the present disclosure includes SPF having an average weight average molecular weight selected from between about 145 to about 150 kDa. In an embodiment, a composition of the present disclosure includes SPF having an average weight average molecular weight selected from between about 150 to about 155 kDa. In an embodiment, a composition of the present disclosure includes SPF having an average weight average molecular weight selected from between about 155 to about 160 kDa. In an embodiment, a composition of the present disclosure includes SPF having an average weight average molecular weight selected from between about 160 to about 165 kDa. In an embodiment, a composition of the present disclosure includes SPF having an average weight average molecular weight selected from between about 165 to about 170 kDa. In an embodiment, a composition of the present disclosure includes SPF having an average weight average molecular weight selected from between about 170 to about 175 kDa. In an embodiment, a composition of the present disclosure includes SPF having an average weight average molecular weight selected from between about 175 to about 180 kDa.

In an embodiment, a composition of the present disclosure includes SPF having an average weight average molecular weight selected from between about 180 to about 185 kDa. In an embodiment, a composition of the present disclosure includes SPF having an average weight average molecular weight selected from between about 185 to about 190 kDa. In an embodiment, a composition of the present disclosure includes SPF having an average weight average molecular weight selected from between about 190 to about 195 kDa. In an embodiment, a composition of the present disclosure includes SPF having an average weight average molecular weight selected from between about 195 to about 200 kDa. In an embodiment, a composition of the present disclosure includes SPF having an average weight average molecular weight selected from between about 200 to about 205 kDa. In an embodiment, a composition of the present disclosure includes SPF having an average weight average molecular weight selected from between about 205 to about 210 kDa. In an embodiment, a composition of the present disclosure includes SPF having an average weight average molecular weight selected from between about 210 to about 215 kDa. In an embodiment, a composition of the present disclosure includes SPF having an average weight average molecular weight selected from between about 215 to about 220 kDa.

In an embodiment, a composition of the present disclosure includes SPF having an average weight average molecular weight selected from between about 220 to about 225 kDa. In an embodiment, a composition of the present disclosure includes SPF having an average weight average molecular weight selected from between about 225 to about 230 kDa. In an embodiment, a composition of the present disclosure includes SPF having an average weight average molecular weight selected from between about 230 to about 235 kDa. In an embodiment, a composition of the present disclosure includes SPF having an average weight average molecular weight selected from between about 235 to about 240 kDa. In an embodiment, a composition of the present disclosure includes SPF having an average weight average molecular weight selected from between about 240 to about 245 kDa. In an embodiment, a composition of the present disclosure includes SPF having an average weight average molecular weight selected from between about 245 to about 250 kDa. In an embodiment, a composition of the present disclosure includes SPF having an average weight average molecular weight selected from between about 250 to about 255 kDa. In an embodiment, a composition of the present disclosure includes SPF having an average weight average molecular weight selected from between about 255 to about 260 kDa.

In an embodiment, a composition of the present disclosure includes SPF having an average weight average molecular weight selected from between about 260 to about 265 kDa. In an embodiment, a composition of the present disclosure includes SPF having an average weight average molecular weight selected from between about 265 to about 270 kDa. In an embodiment, a composition of the present disclosure includes SPF having an average weight average molecular weight selected from between about 270 to about 275 kDa. In an embodiment, a composition of the present disclosure includes SPF having an average weight average molecular weight selected from between about 275 to about 280 kDa. In an embodiment, a composition of the present disclosure includes SPF having an average weight average molecular weight selected from between about 280 to about 285 kDa. In an embodiment, a composition of the present disclosure includes SPF having an average weight average molecular weight selected from between about 285 to about 290 kDa. In an embodiment, a composition of the present disclosure includes SPF having an average weight average molecular weight selected from between about 290 to about 295 kDa. In an embodiment, a composition of the present disclosure includes SPF having an average weight average molecular weight selected from between about 295 to about 300 kDa.

In an embodiment, a composition of the present disclosure includes SPF having an average weight average molecular weight selected from between about 300 to about 305 kDa. In an embodiment, a composition of the present disclosure includes SPF having an average weight average molecular weight selected from between about 305 to about 310 kDa. In an embodiment, a composition of the present disclosure includes SPF having an average weight average molecular weight selected from between about 310 to about 315 kDa. In an embodiment, a composition of the present disclosure includes SPF having an average weight average molecular weight selected from between about 315 to about 320 kDa. In an embodiment, a composition of the present disclosure includes SPF having an average weight average molecular weight selected from between about 320 to about 325 kDa. In an embodiment, a composition of the present disclosure includes SPF having an average weight average molecular weight selected from between about 325 to about 330 kDa. In an embodiment, a composition of the present disclosure includes SPF having an average weight average molecular weight selected from between about 330 to about 335 kDa. In an embodiment, a composition of the present disclosure includes SPF having an average weight average molecular weight selected from between about 335 to about 340 kDa.

In an embodiment, a composition of the present disclosure includes SPF having an average weight average molecular weight selected from between about 340 to about 345 kDa. In an embodiment, a composition of the present disclosure includes SPF having an average weight average molecular weight selected from between about 345 to about 350 kDa.

In some embodiments, compositions of the present disclosure include SPF compositions selected from compositions #1001 to #2450, having weight average molecular weights selected from about 1 kDa to about 145 kDa, and a polydispersity selected from between 1 and about 5 (including, without limitation, a polydispersity of 1), between 1 and about 1.5 (including, without limitation, a polydispersity of 1), between about 1.5 and about 2, between about 1.5 and about 3, between about 2 and about 2.5, between about 2.5 and about 3, between about 3 and about 3.5, between about 3.5 and about 4, between about 4 and about 4.5, and between about 4.5 and about 5:

| | | PDI | | | | | | | | | |
|---|---|---|---|---|---|---|---|---|---|---|---|
| MW | | (about) | | | | | | | | | |
| (about) | | 1-5 | 1-1.5 | 1.5-2 | 1.5-3 | 2-2.5 | 2.5-3 | 3-3.5 | 3.5-4 | 4-4.5 | 4.5-5 |
| 1 | kDa | 1001 | 1002 | 1003 | 1004 | 1005 | 1006 | 1007 | 1008 | 1009 | 1010 |
| 2 | kDa | 1011 | 1012 | 1013 | 1014 | 1015 | 1016 | 1017 | 1018 | 1019 | 1020 |
| 3 | kDa | 1021 | 1022 | 1023 | 1024 | 1025 | 1026 | 1027 | 1028 | 1029 | 1030 |
| 4 | kDa | 1031 | 1032 | 1033 | 1034 | 1035 | 1036 | 1037 | 1038 | 1039 | 1040 |
| 5 | kDa | 1041 | 1042 | 1043 | 1044 | 1045 | 1046 | 1047 | 1048 | 1049 | 1050 |
| 6 | kDa | 1051 | 1052 | 1053 | 1054 | 1055 | 1056 | 1057 | 1058 | 1059 | 1060 |
| 7 | kDa | 1061 | 1062 | 1063 | 1064 | 1065 | 1066 | 1067 | 1068 | 1069 | 1070 |
| 8 | kDa | 1071 | 1072 | 1073 | 1074 | 1075 | 1076 | 1077 | 1078 | 1079 | 1080 |
| 9 | kDa | 1081 | 1082 | 1083 | 1084 | 1085 | 1086 | 1087 | 1088 | 1089 | 1090 |
| 10 | kDa | 1091 | 1092 | 1093 | 1094 | 1095 | 1096 | 1097 | 1098 | 1099 | 1100 |
| 11 | kDa | 1101 | 1102 | 1103 | 1104 | 1105 | 1106 | 1107 | 1108 | 1109 | 1110 |
| 12 | kDa | 1111 | 1112 | 1113 | 1114 | 1115 | 1116 | 1117 | 1118 | 1119 | 1120 |
| 13 | kDa | 1121 | 1122 | 1123 | 1124 | 1125 | 1126 | 1127 | 1128 | 1129 | 1130 |
| 14 | kDa | 1131 | 1132 | 1133 | 1134 | 1135 | 1136 | 1137 | 1138 | 1139 | 1140 |
| 15 | kDa | 1141 | 1142 | 1143 | 1144 | 1145 | 1146 | 1147 | 1148 | 1149 | 1150 |
| 16 | kDa | 1151 | 1152 | 1153 | 1154 | 1155 | 1156 | 1157 | 1158 | 1159 | 1160 |
| 17 | kDa | 1161 | 1162 | 1163 | 1164 | 1165 | 1166 | 1167 | 1168 | 1169 | 1170 |
| 18 | kDa | 1171 | 1172 | 1173 | 1174 | 1175 | 1176 | 1177 | 1178 | 1179 | 1180 |
| 19 | kDa | 1181 | 1182 | 1183 | 1184 | 1185 | 1186 | 1187 | 1188 | 1189 | 1190 |
| 20 | kDa | 1191 | 1192 | 1193 | 1194 | 1195 | 1196 | 1197 | 1198 | 1199 | 1200 |
| 21 | kDa | 1201 | 1202 | 1203 | 1204 | 1205 | 1206 | 1207 | 1208 | 1209 | 1210 |
| 22 | kDa | 1211 | 1212 | 1213 | 1214 | 1215 | 1216 | 1217 | 1218 | 1219 | 1220 |
| 23 | kDa | 1221 | 1222 | 1223 | 1224 | 1225 | 1226 | 1227 | 1228 | 1229 | 1230 |
| 24 | kDa | 1231 | 1232 | 1233 | 1234 | 1235 | 1236 | 1237 | 1238 | 1239 | 1240 |
| 25 | kDa | 1241 | 1242 | 1243 | 1244 | 1245 | 1246 | 1247 | 1248 | 1249 | 1250 |
| 26 | kDa | 1251 | 1252 | 1253 | 1254 | 1255 | 1256 | 1257 | 1258 | 1259 | 1260 |
| 27 | kDa | 1261 | 1262 | 1263 | 1264 | 1265 | 1266 | 1267 | 1268 | 1269 | 1270 |
| 28 | kDa | 1271 | 1272 | 1273 | 1274 | 1275 | 1276 | 1277 | 1278 | 1279 | 1280 |
| 29 | kDa | 1281 | 1282 | 1283 | 1284 | 1285 | 1286 | 1287 | 1288 | 1289 | 1290 |
| 30 | kDa | 1291 | 1292 | 1293 | 1294 | 1295 | 1296 | 1297 | 1298 | 1299 | 1300 |
| 31 | kDa | 1301 | 1302 | 1303 | 1304 | 1305 | 1306 | 1307 | 1308 | 1309 | 1310 |
| 32 | kDa | 1311 | 1312 | 1313 | 1314 | 1315 | 1316 | 1317 | 1318 | 1319 | 1320 |
| 33 | kDa | 1321 | 1322 | 1323 | 1324 | 1325 | 1326 | 1327 | 1328 | 1329 | 1330 |
| 34 | kDa | 1331 | 1332 | 1333 | 1334 | 1335 | 1336 | 1337 | 1338 | 1339 | 1340 |
| 35 | kDa | 1341 | 1342 | 1343 | 1344 | 1345 | 1346 | 1347 | 1348 | 1349 | 1350 |
| 36 | kDa | 1351 | 1352 | 1353 | 1354 | 1355 | 1356 | 1357 | 1358 | 1359 | 1360 |
| 37 | kDa | 1361 | 1362 | 1363 | 1364 | 1365 | 1366 | 1367 | 1368 | 1369 | 1370 |
| 38 | kDa | 1371 | 1372 | 1373 | 1374 | 1375 | 1376 | 1377 | 1378 | 1379 | 1380 |
| 39 | kDa | 1381 | 1382 | 1383 | 1384 | 1385 | 1386 | 1387 | 1388 | 1389 | 1390 |
| 40 | kDa | 1391 | 1392 | 1393 | 1394 | 1395 | 1396 | 1397 | 1398 | 1399 | 1400 |
| 41 | kDa | 1401 | 1402 | 1403 | 1404 | 1405 | 1406 | 1407 | 1408 | 1409 | 1410 |
| 42 | kDa | 1411 | 1412 | 1413 | 1414 | 1415 | 1416 | 1417 | 1418 | 1419 | 1420 |
| 43 | kDa | 1421 | 1422 | 1423 | 1424 | 1425 | 1426 | 1427 | 1428 | 1429 | 1430 |
| 44 | kDa | 1431 | 1432 | 1433 | 1434 | 1435 | 1436 | 1437 | 1438 | 1439 | 1440 |
| 45 | kDa | 1441 | 1442 | 1443 | 1444 | 1445 | 1446 | 1447 | 1448 | 1449 | 1450 |
| 46 | kDa | 1451 | 1452 | 1453 | 1454 | 1455 | 1456 | 1457 | 1458 | 1459 | 1460 |
| 47 | kDa | 1461 | 1462 | 1463 | 1464 | 1465 | 1466 | 1467 | 1468 | 1469 | 1470 |
| 48 | kDa | 1471 | 1472 | 1473 | 1474 | 1475 | 1476 | 1477 | 1478 | 1479 | 1480 |
| 49 | kDa | 1481 | 1482 | 1483 | 1484 | 1485 | 1486 | 1487 | 1488 | 1489 | 1490 |
| 50 | kDa | 1491 | 1492 | 1493 | 1494 | 1495 | 1496 | 1497 | 1498 | 1499 | 1500 |
| 51 | kDa | 1501 | 1502 | 1503 | 1504 | 1505 | 1506 | 1507 | 1508 | 1509 | 1510 |
| 52 | kDa | 1511 | 1512 | 1513 | 1514 | 1515 | 1516 | 1517 | 1518 | 1519 | 1520 |
| 53 | kDa | 1521 | 1522 | 1523 | 1524 | 1525 | 1526 | 1527 | 1528 | 1529 | 1530 |
| 54 | kDa | 1531 | 1532 | 1533 | 1534 | 1535 | 1536 | 1537 | 1538 | 1539 | 1540 |
| 55 | kDa | 1541 | 1542 | 1543 | 1544 | 1545 | 1546 | 1547 | 1548 | 1549 | 1550 |
| 56 | kDa | 1551 | 1552 | 1553 | 1554 | 1555 | 1556 | 1557 | 1558 | 1559 | 1560 |
| 57 | kDa | 1561 | 1562 | 1563 | 1564 | 1565 | 1566 | 1567 | 1568 | 1569 | 1570 |
| 58 | kDa | 1571 | 1572 | 1573 | 1574 | 1575 | 1576 | 1577 | 1578 | 1579 | 1580 |
| 59 | kDa | 1581 | 1582 | 1583 | 1584 | 1585 | 1586 | 1587 | 1588 | 1589 | 1590 |
| 60 | kDa | 1591 | 1592 | 1593 | 1594 | 1595 | 1596 | 1597 | 1598 | 1599 | 1600 |
| 61 | kDa | 1601 | 1602 | 1603 | 1604 | 1605 | 1606 | 1607 | 1608 | 1609 | 1610 |
| 62 | kDa | 1611 | 1612 | 1613 | 1614 | 1615 | 1616 | 1617 | 1618 | 1619 | 1620 |
| 63 | kDa | 1621 | 1622 | 1623 | 1624 | 1625 | 1626 | 1627 | 1628 | 1629 | 1630 |
| 64 | kDa | 1631 | 1632 | 1633 | 1634 | 1635 | 1636 | 1637 | 1638 | 1639 | 1640 |
| 65 | kDa | 1641 | 1642 | 1643 | 1644 | 1645 | 1646 | 1647 | 1648 | 1649 | 1650 |
| 66 | kDa | 1651 | 1652 | 1653 | 1654 | 1655 | 1656 | 1657 | 1658 | 1659 | 1660 |
| 67 | kDa | 1661 | 1662 | 1663 | 1664 | 1665 | 1666 | 1667 | 1668 | 1669 | 1670 |
| 68 | kDa | 1671 | 1672 | 1673 | 1674 | 1675 | 1676 | 1677 | 1678 | 1679 | 1680 |
| 69 | kDa | 1681 | 1682 | 1683 | 1684 | 1685 | 1686 | 1687 | 1688 | 1689 | 1690 |
| 70 | kDa | 1691 | 1692 | 1693 | 1694 | 1695 | 1696 | 1697 | 1698 | 1699 | 1700 |
| 71 | kDa | 1701 | 1702 | 1703 | 1704 | 1705 | 1706 | 1707 | 1708 | 1709 | 1710 |
| 72 | kDa | 1711 | 1712 | 1713 | 1714 | 1715 | 1716 | 1717 | 1718 | 1719 | 1720 |
| 73 | kDa | 1721 | 1722 | 1723 | 1724 | 1725 | 1726 | 1727 | 1728 | 1729 | 1730 |
| 74 | kDa | 1731 | 1732 | 1733 | 1734 | 1735 | 1736 | 1737 | 1738 | 1739 | 1740 |
| 75 | kDa | 1741 | 1742 | 1743 | 1744 | 1745 | 1746 | 1747 | 1748 | 1749 | 1750 |
| 76 | kDa | 1751 | 1752 | 1753 | 1754 | 1755 | 1756 | 1757 | 1758 | 1759 | 1760 |
| 77 | kDa | 1761 | 1762 | 1763 | 1764 | 1765 | 1766 | 1767 | 1768 | 1769 | 1770 |
| 78 | kDa | 1771 | 1772 | 1773 | 1774 | 1775 | 1776 | 1777 | 1778 | 1779 | 1780 |
| 79 | kDa | 1781 | 1782 | 1783 | 1784 | 1785 | 1786 | 1787 | 1788 | 1789 | 1790 |
| 80 | kDa | 1791 | 1792 | 1793 | 1794 | 1795 | 1796 | 1797 | 1798 | 1799 | 1800 |
| 81 | kDa | 1801 | 1802 | 1803 | 1804 | 1805 | 1806 | 1807 | 1808 | 1809 | 1810 |
| 82 | kDa | 1811 | 1812 | 1813 | 1814 | 1815 | 1816 | 1817 | 1818 | 1819 | 1820 |
| 83 | kDa | 1821 | 1822 | 1823 | 1824 | 1825 | 1826 | 1827 | 1828 | 1829 | 1830 |
| 84 | kDa | 1831 | 1832 | 1833 | 1834 | 1835 | 1836 | 1837 | 1838 | 1839 | 1840 |
| 85 | kDa | 1841 | 1842 | 1843 | 1844 | 1845 | 1846 | 1847 | 1848 | 1849 | 1850 |
| 86 | kDa | 1851 | 1852 | 1853 | 1854 | 1855 | 1856 | 1857 | 1858 | 1859 | 1860 |
| 87 | kDa | 1861 | 1862 | 1863 | 1864 | 1865 | 1866 | 1867 | 1868 | 1869 | 1870 |
| 88 | kDa | 1871 | 1872 | 1873 | 1874 | 1875 | 1876 | 1877 | 1878 | 1879 | 1880 |
| 89 | kDa | 1881 | 1882 | 1883 | 1884 | 1885 | 1886 | 1887 | 1888 | 1889 | 1890 |
| 90 | kDa | 1891 | 1892 | 1893 | 1894 | 1895 | 1896 | 1897 | 1898 | 1899 | 1900 |
| 91 | kDa | 1901 | 1902 | 1903 | 1904 | 1905 | 1906 | 1907 | 1908 | 1909 | 1910 |
| 92 | kDa | 1911 | 1912 | 1913 | 1914 | 1915 | 1916 | 1917 | 1918 | 1919 | 1920 |
| 93 | kDa | 1921 | 1922 | 1923 | 1924 | 1925 | 1926 | 1927 | 1928 | 1929 | 1930 |
| 94 | kDa | 1931 | 1932 | 1933 | 1934 | 1935 | 1936 | 1937 | 1938 | 1939 | 1940 |
| 95 | kDa | 1941 | 1942 | 1943 | 1944 | 1945 | 1946 | 1947 | 1948 | 1949 | 1950 |
| 96 | kDa | 1951 | 1952 | 1953 | 1954 | 1955 | 1956 | 1957 | 1958 | 1959 | 1960 |
| 97 | kDa | 1961 | 1962 | 1963 | 1964 | 1965 | 1966 | 1967 | 1968 | 1969 | 1970 |
| 98 | kDa | 1971 | 1972 | 1973 | 1974 | 1975 | 1976 | 1977 | 1978 | 1979 | 1980 |
| 99 | kDa | 1981 | 1982 | 1983 | 1984 | 1985 | 1986 | 1987 | 1988 | 1989 | 1990 |
| 100 | kDa | 1991 | 1992 | 1993 | 1994 | 1995 | 1996 | 1997 | 1998 | 1999 | 2000 |
| 101 | kDa | 2001 | 2002 | 2003 | 2004 | 2005 | 2006 | 2007 | 2008 | 2009 | 2010 |
| 102 | kDa | 2011 | 2012 | 2013 | 2014 | 2015 | 2016 | 2017 | 2018 | 2019 | 2020 |
| 103 | kDa | 2021 | 2022 | 2023 | 2024 | 2025 | 2026 | 2027 | 2028 | 2029 | 2030 |
| 104 | kDa | 2031 | 2032 | 2033 | 2034 | 2035 | 2036 | 2037 | 2038 | 2039 | 2040 |
| 105 | kDa | 2041 | 2042 | 2043 | 2044 | 2045 | 2046 | 2047 | 2048 | 2049 | 2050 |
| 106 | kDa | 2051 | 2052 | 2053 | 2054 | 2055 | 2056 | 2057 | 2058 | 2059 | 2060 |
| 107 | kDa | 2061 | 2062 | 2063 | 2064 | 2065 | 2066 | 2067 | 2068 | 2069 | 2070 |
| 108 | kDa | 2071 | 2072 | 2073 | 2074 | 2075 | 2076 | 2077 | 2078 | 2079 | 2080 |
| 109 | kDa | 2081 | 2082 | 2083 | 2084 | 2085 | 2086 | 2087 | 2088 | 2089 | 2090 |
| 110 | kDa | 2091 | 2092 | 2093 | 2094 | 2095 | 2096 | 2097 | 2098 | 2099 | 2100 |
| 111 | kDa | 2101 | 2102 | 2103 | 2104 | 2105 | 2106 | 2107 | 2108 | 2109 | 2110 |
| 112 | kDa | 2111 | 2112 | 2113 | 2114 | 2115 | 2116 | 2117 | 2118 | 2119 | 2120 |
| 113 | kDa | 2121 | 2122 | 2123 | 2124 | 2125 | 2126 | 2127 | 2128 | 2129 | 2130 |
| 114 | kDa | 2131 | 2132 | 2133 | 2134 | 2135 | 2136 | 2137 | 2138 | 2139 | 2140 |
| 115 | kDa | 2141 | 2142 | 2143 | 2144 | 2145 | 2146 | 2147 | 2148 | 2149 | 2150 |
| 116 | kDa | 2151 | 2152 | 2153 | 2154 | 2155 | 2156 | 2157 | 2158 | 2159 | 2160 |
| 117 | kDa | 2161 | 2162 | 2163 | 2164 | 2165 | 2166 | 2167 | 2168 | 2169 | 2170 |
| 118 | kDa | 2171 | 2172 | 2173 | 2174 | 2175 | 2176 | 2177 | 2178 | 2179 | 2180 |
| 119 | kDa | 2181 | 2182 | 2183 | 2184 | 2185 | 2186 | 2187 | 2188 | 2189 | 2180 |
| 120 | kDa | 2191 | 2192 | 2193 | 2194 | 2195 | 2196 | 2197 | 2198 | 2199 | 2190 |
| 121 | kDa | 2201 | 2202 | 2203 | 2204 | 2205 | 2206 | 2207 | 2208 | 2209 | 2200 |
| 122 | kDa | 2211 | 2212 | 2213 | 2214 | 2215 | 2216 | 2217 | 2218 | 2219 | 2210 |
| 123 | kDa | 2221 | 2222 | 2223 | 2224 | 2225 | 2226 | 2227 | 2228 | 2229 | 2220 |
| 124 | kDa | 2231 | 2232 | 2233 | 2234 | 2235 | 2236 | 2237 | 2238 | 2239 | 2230 |
| 125 | kDa | 2241 | 2242 | 2243 | 2244 | 2245 | 2246 | 2247 | 2248 | 2249 | 2240 |
| 126 | kDa | 2251 | 2252 | 2253 | 2254 | 2255 | 2256 | 2257 | 2258 | 2259 | 2250 |
| 127 | kDa | 2261 | 2262 | 2263 | 2264 | 2265 | 2266 | 2267 | 2268 | 2269 | 2260 |
| 128 | kDa | 2271 | 2272 | 2273 | 2274 | 2275 | 2276 | 2277 | 2278 | 2279 | 2270 |
| 129 | kDa | 2281 | 2282 | 2283 | 2284 | 2285 | 2286 | 2287 | 2288 | 2289 | 2280 |
| 130 | kDa | 2291 | 2292 | 2293 | 2294 | 2295 | 2296 | 2297 | 2298 | 2299 | 2290 |
| 131 | kDa | 2301 | 2302 | 2303 | 2304 | 2305 | 2306 | 2307 | 2308 | 2309 | 2300 |
| 132 | kDa | 2311 | 2312 | 2313 | 2314 | 2315 | 2316 | 2317 | 2318 | 2319 | 2310 |
| 133 | kDa | 2321 | 2322 | 2323 | 2324 | 2325 | 2326 | 2327 | 2328 | 2329 | 2320 |
| 134 | kDa | 2331 | 2332 | 2333 | 2334 | 2335 | 2336 | 2337 | 2338 | 2339 | 2330 |
| 135 | kDa | 2341 | 2342 | 2343 | 2344 | 2345 | 2346 | 2347 | 2348 | 2349 | 2340 |
| 136 | kDa | 2351 | 2352 | 2353 | 2354 | 2355 | 2356 | 2357 | 2358 | 2359 | 2350 |
| 137 | kDa | 2361 | 2362 | 2363 | 2364 | 2365 | 2366 | 2367 | 2368 | 2369 | 2360 |
| 138 | kDa | 2371 | 2372 | 2373 | 2374 | 2375 | 2376 | 2377 | 2378 | 2379 | 2370 |
| 139 | kDa | 2381 | 2382 | 2383 | 2384 | 2385 | 2386 | 2387 | 2388 | 2389 | 2380 |
| 140 | kDa | 2391 | 2392 | 2393 | 2394 | 2395 | 2396 | 2397 | 2398 | 2399 | 2390 |
| 141 | kDa | 2401 | 2402 | 2403 | 2404 | 2405 | 2406 | 2407 | 2408 | 2409 | 2400 |
| 142 | kDa | 2411 | 2412 | 2413 | 2414 | 2415 | 2416 | 2417 | 2418 | 2419 | 2410 |
| 143 | kDa | 2421 | 2422 | 2423 | 2424 | 2525 | 2526 | 2527 | 2528 | 2529 | 2520 |
| 144 | kDa | 2431 | 2432 | 2433 | 2434 | 2535 | 2536 | 2537 | 2538 | 2539 | 2530 |
| 145 | kDa | 2441 | 2442 | 2443 | 2444 | 2445 | 2446 | 2447 | 2448 | 2449 | 2440 |

In an embodiment, SPF in a composition of the present disclosure have a polydispersity selected from between 1 to about 5.0, including, without limitation, a polydispersity of 1. In an embodiment, SPF in a composition of the present disclosure have a polydispersity selected from between about 1.5 to about 3.0. In an embodiment, SPF in a composition of the present disclosure have a polydispersity selected from between 1 to about 1.5, including, without limitation, a polydispersity of 1. In an embodiment, SPF in a composition of the present disclosure have a polydispersity selected from between about 1.5 to about 2.0. In an embodiment, SPF in a composition of the present disclosure have a polydispersity selected from between about 2.0 to about 2.5. In an embodiment, SPF in a composition of the present disclosure have a polydispersity selected from between about 2.5 to about 3.0.

In an embodiment, SPF in a composition of the present disclosure have a polydispersity selected from between about 3.0 to about 3.5. In an embodiment, SPF in a composition of the present disclosure have a polydispersity selected from between about 3.5 to about 4.0. In an embodiment, SPF in a composition of the present disclosure have a polydispersity selected from between about 4.0 to about 4.5. In an embodiment, SPF in a composition of the present disclosure have a polydispersity selected from between about 4.5 to about 5.0.

In an embodiment, SPF in a composition of the present disclosure have a polydispersity of 1. In an embodiment, SPF in a composition of the present disclosure have a polydispersity of about 1.1. In an embodiment, SPF in a composition of the present disclosure have a polydispersity of about 1.2. In an embodiment, SPF in a composition of the present disclosure have a polydispersity of about 1.3. In an embodiment, SPF in a composition of the present disclosure have a polydispersity of about 1.4. In an embodiment, SPF in a composition of the present disclosure have a polydispersity of about 1.5. In an embodiment, SPF in a composition of the present disclosure have a polydispersity of about 1.6. In an embodiment, SPF in a composition of the present disclosure have a polydispersity of about 1.7. In an embodiment, SPF in a composition of the present disclosure have a polydispersity of about 1.8. In an embodiment, SPF in a composition of the present disclosure have a polydispersity of about 1.9. In an embodiment, SPF in a composition of the present disclosure have a polydispersity of about 2.0. In an embodiment, SPF in a composition of the present disclosure have a polydispersity of about 2.1. In an embodiment, SPF in a composition of the present disclosure have a polydispersity of about 2.2. In an embodiment, SPF in a composition of the present disclosure have a polydispersity of about 2.3. In an embodiment, SPF in a composition of the present disclosure have a polydispersity of about 2.4. In an embodiment, SPF in a composition of the present disclosure have a polydispersity of about 2.5. In an embodiment, SPF in a composition of the present disclosure have a polydispersity of about 2.6. In an embodiment, SPF in a composition of the present disclosure have a polydispersity of about 2.7. In an embodiment, SPF in a composition of the present disclosure have a polydispersity of about 2.8. In an embodiment, SPF in a composition of the present disclosure have a polydispersity of about 2.9. In an embodiment, SPF in a composition of the present disclosure have a polydispersity of about 3.0. In an embodiment, SPF in a composition of the present disclosure have a polydispersity of about 3.1. In an embodiment, SPF in a composition of the present disclosure have a polydispersity of about 3.2. In an embodiment, SPF in a composition of the present disclosure have a polydispersity of about 3.3. In an embodiment, SPF in a composition of the present disclosure have a polydispersity of about 3.4. In an embodiment, SPF in a composition of the present disclosure have a polydispersity of about 3.5. In an embodiment, SPF in a composition of the present disclosure have a polydispersity of about 3.6. In an embodiment, SPF in a composition of the present disclosure have a polydispersity of about 3.7. In an embodiment, SPF in a composition of the present disclosure have a polydispersity of about 3.8. In an embodiment, SPF in a composition of the present disclosure have a polydispersity of about 3.9. In an embodiment, SPF in a composition of the present disclosure have a polydispersity of about 4.0. In an embodiment, SPF in a composition of the present disclosure have a polydispersity of about 4.1. In an embodiment, SPF in a composition of the present disclosure have a polydispersity of about 4.2. In an embodiment, SPF in a composition of the present disclosure have a polydispersity of about 4.3. In an embodiment, SPF in a composition of the present disclosure have a polydispersity of about 4.4. In an embodiment, SPF in a composition of the present disclosure have a polydispersity of about 4.5. In an embodiment, SPF in a composition of the present disclosure have a polydispersity of about 4.6. In an embodiment, SPF in a composition of the present disclosure have a polydispersity of about 4.7. In an embodiment, SPF in a composition of the present disclosure have a polydispersity of about 4.8. In an embodiment, SPF in a composition of the present disclosure have a polydispersity of about 4.9. In an embodiment, SPF in a composition of the present disclosure have a polydispersity of about 5.0.

### Silk Fibroin Fragments

Methods of making silk fibroin or silk fibroin protein fragments and their applications in various fields are known and are described for example in U.S. Pat. Nos. 9,187,538, 9,511,012, 9,517,191, 9,522,107, 9,522,108, 9,545,369, and 10,166,177, 10,287,728 and 10,301,768, all of which are incorporated herein in their entireties. Raw silk from silkworm Bombyx mori is composed of two primary proteins: silk fibroin (approximately 75%) and sericin (approximately 25%). Silk fibroin is a fibrous protein with a semi-crystalline structure that provides stiffness and strength. As used herein, the term “silk fibroin” means the fibers of the cocoon of Bombyx mori having a weight average molecular weight of about 370,000 Da. The crude silkworm fiber consists of a double thread of fibroin. The adhesive substance holding these double fibers together is sericin. The silk fibroin is composed of a heavy chain having a weight average molecular weight of about 350,000 Da (H chain), and a light chain having a weight average molecular weight about 25,000 Da (L chain). Silk fibroin is an amphiphilic polymer with large hydrophobic domains occupying the major component of the polymer, which has a high molecular weight. The hydrophobic regions are interrupted by small hydrophilic spacers, and the N- and C-termini of the chains are also highly hydrophilic. The hydrophobic domains of the H-chain contain a repetitive hexapeptide sequence of Gly-Ala-Gly-Ala-Gly-Ser and repeats of Gly-Ala/Ser/Tyr dipeptides, which can form stable anti-parallel-sheet crystallites. The amino acid sequence of the L-chain is non-repetitive, so the L-chain is more hydrophilic and relatively elastic. The hydrophilic (Tyr, Ser) and hydrophobic (Gly, Ala) chain segments in silk fibroin molecules are arranged alternatively such that allows self-assembling of silk fibroin molecules.

Provided herein are methods for producing pure and highly scalable silk fibroin-protein fragment mixture solutions that may be used across multiple industries for a variety of applications. Without wishing to be bound by any particular theory, it is believed that these methods are equally applicable to fragmentation of any SPF described herein, including without limitation recombinant silk proteins, and fragmentation of silk-like or fibroin-like proteins.

As used herein, the term “fibroin” includes silk worm fibroin and insect or spider silk protein. In an embodiment, fibroin is obtained from Bombyx mori. Raw silk from Bombyx mori is composed of two primary proteins: silk fibroin (approximately 75%) and sericin (approximately 25%). Silk fibroin is a fibrous protein with a semi-crystalline structure that provides stiffness and strength. As used herein, the term “silk fibroin” means the fibers of the cocoon of Bombyx mori having a weight average molecular weight of about 370,000 Da. Conversion of these insoluble silk fibroin fibrils into water-soluble silk fibroin protein fragments requires the addition of a concentrated neutral salt (e.g., 8-10 M lithium bromide), which interferes with inter- and intramolecular ionic and hydrogen bonding that would otherwise render the fibroin protein insoluble in water. Methods of making silk fibroin protein fragments, and/or compositions thereof, are known and are described for example in U.S. Pat. Nos. 9,187,538, 9,511,012, 9,517,191, 9,522,107, 9,522,108, 9,545,369, and 10,166,177.

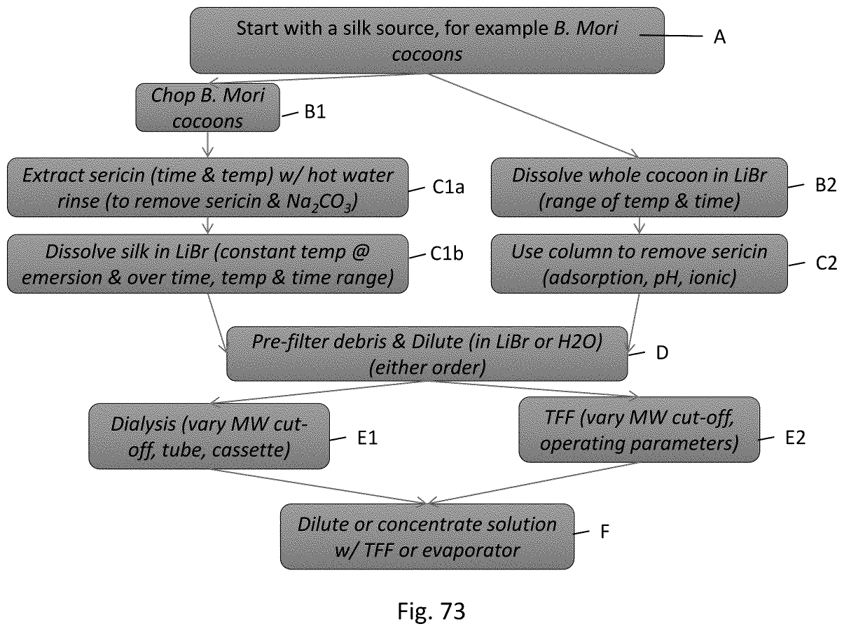

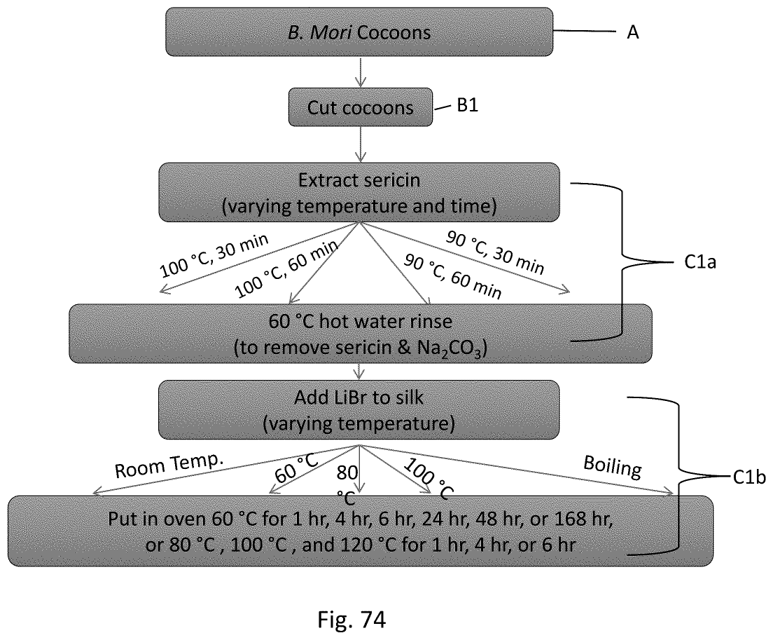

FIG. 73 is a flow chart showing generic embodiments for producing some embodiments of the present disclosure. It should be understood that not all of the steps illustrated are necessarily required to fabricate all silk solutions of the present disclosure. As illustrated in FIG. 74, step A, cocoons (heat-treated or non-heat-treated), silk fibers, silk powder, spider silk or recombinant spider silk can be used as the silk source.

FIG. 74 is a flow chart showing generic embodiments for various parameters that can be modified during the process of producing a silk protein fragment solution of the present disclosure during the extraction and the dissolution steps.

In some embodiments, specifically disclosed herein, molecular weight of the silk fibroin protein fragment composition was determined using High Pressure Liquid Chromatography (HPLC) with a Refractive Index Detector (RID), and polydispersity was calculated using Cirrus GPC Online GPC/SEC Software Version 3.3 (Agilent). In some embodiments specifically disclosed herein, molecular weight of the silk fibroin protein fragment composition was determined using SEC-MALS methods.

### Data Analysis and Calculations—Calculation of Average Molecular Weight Using Cirrus Software

Upload the chromatography data files of the standards and the analytical samples into Cirrus SEC data collection and molecular weight analysis software. Calculate the weight average molecular weight (M_{w}), number average molecular weight (Mₙ), peak average molecular weight (Mₚ), and polydispersity for each injection of the sample.

### Spider Silk Fragments

Spider silks are natural polymers that consist of three domains: a repetitive middle core domain that dominates the protein chain, and non-repetitive N-terminal and C-terminal domains. The large core domain is organized in a block copolymer-like arrangement, in which two basic sequences, crystalline [poly(A) or poly(GA)] and less crystalline (GGX or GPGXX (SEQ ID NO:7)) polypeptides alternate. Dragline silk is the protein complex composed of major ampullate dragline silk protein 1 (MaSp1) and major ampullate dragline silk protein 2 (MaSp2). Both silks are approximately 3500 amino acid long. MaSp1 can be found in the fiber core and the periphery, whereas MaSp2 forms clusters in certain core areas. The large central domains of MaSp1 and MaSp2 are organized in block copolymer-like arrangements, in which two basic sequences, crystalline [poly(A) or poly(GA)] and less crystalline (GGX or GPGXX (SEQ ID NO:7)) polypeptides alternate in core domain. Specific secondary structures have been assigned to poly(A)/(GA), GGX and GPGXX(SEQ ID NO:7) motifs including β-sheet, α-helix and β-spiral respectively. The primary sequence, composition and secondary structural elements of the repetitive core domain are responsible for mechanical properties of spider silks; whereas, non-repetitive N- and C-terminal domains are essential for the storage of liquid silk dope in a lumen and fiber formation in a spinning duct.

The main difference between MaSp1 and MaSp2 is the presence of proline (P) residues accounting for 15% of the total amino acid content in MaSp2, whereas MaSp1 is proline-free. By calculating the number of proline residues in N. clavipes dragline silk, it is possible to estimate the presence of the two proteins in fibers; 81% MaSp1 and 19% MaSp2. Different spiders have different ratios of MaSp1 and MaSp2. For example, a dragline silk fiber from the orb weaver Argiope aurantia contains 41% MaSp1 and 59% MaSp2. Such changes in the ratios of major ampullate silks can dictate the performance of the silk fiber.

At least seven different types of silk proteins are known for one orb-weaver species of spider. Silks differ in primary sequence, physical properties and functions. For example, dragline silks used to build frames, radii and lifelines are known for outstanding mechanical properties including strength, toughness and elasticity. On an equal weight basis, spider silk has a higher toughness than steel and Kevlar.

Flageliform silk found in capture spirals has extensibility of up to 500%. Minor ampullate silk, which is found in auxiliary spirals of the orb-web and in prey wrapping, possesses high toughness and strength almost similar to major ampullate silks, but does not supercontract in water.

Spider silks are known for their high tensile strength and toughness. The recombinant silk proteins also confer advantageous properties to cosmetic or dermatological compositions, in particular to be able to improve the hydrating or softening action, good film forming property and low surface density. Diverse and unique biomechanical properties together with biocompatibility and a slow rate of degradation make spider silks excellent candidates as biomaterials for tissue engineering, guided tissue repair and drug delivery, for cosmetic products (e.g. nail and hair strengthener, skin care products), and industrial materials (e.g. nanowires, nanofibers, surface coatings).

In an embodiment, a silk protein may include a polypeptide derived from natural spider silk proteins. The polypeptide is not limited particularly as long as it is derived from natural spider silk proteins, and examples of the polypeptide include natural spider silk proteins and recombinant spider silk proteins such as variants, analogs, derivatives or the like of the natural spider silk proteins. In terms of excellent tenacity, the polypeptide may be derived from major dragline silk proteins produced in major ampullate glands of spiders. Examples of the major dragline silk proteins include major ampullate spidroin MaSp1 and MaSp2 from Nephila clavipes, and ADF3 and ADF4 from Araneus diadematus, etc. Examples of the polypeptide derived from major dragline silk proteins include variants, analogs, derivatives or the like of the major dragline silk proteins. Further, the polypeptide may be derived from flagelliform silk proteins produced in flagelliform glands of spiders. Examples of the flagelliform silk proteins include flagelliform silk proteins derived from Nephila clavipes, etc.

Examples of the polypeptide derived from major dragline silk proteins include a polypeptide containing two or more units of an amino acid sequence represented by the formula 1: REP1-REP2 (1), preferably a polypeptide containing five or more units thereof, and more preferably a polypeptide containing ten or more units thereof. Alternatively, the polypeptide derived from major dragline silk proteins may be a polypeptide that contains units of the amino acid sequence represented by the formula 1: REP1-REP2 (1) and that has, at a C-terminal, an amino acid sequence represented by any of SEQ ID NOS: 1 to 3 of U.S. Pat. No. 9,051,453, which is incorporated by reference herein in its entirety, or an amino acid sequence having a homology of 90% or more with the amino acid sequence represented by any of SEQ ID NOS: 1 to 3 of U.S. Pat. No. 9,051,453, which is incorporated by reference herein in its entirety. In the polypeptide derived from major dragline silk proteins, units of the amino acid sequence represented by the formula 1: REP1-REP2 (1) may be the same or may be different from each other. In the case of producing a recombinant protein using a microbe such as Escherichia coli as a host, the molecular weight of the polypeptide derived from major dragline silk proteins is 500 kDa or less, or 300 kDa or less, or 200 kDa or less, in terms of productivity.

In the formula (1), the REP1 indicates polyalanine. In the REP1, the number of alanine residues arranged in succession is preferably 2 or more, more preferably 3 or more, further preferably 4 or more, and particularly preferably 5 or more. Further, in the REP1, the number of alanine residues arranged in succession is preferably 20 or less, more preferably 16 or less, further preferably 12 or less, and particularly preferably 10 or less. In the formula (1), the REP2 is an amino acid sequence composed of 10 to 200 amino acid residues. The total number of glycine, serine, glutamine and alanine residues contained in the amino acid sequence is 40% or more, preferably 60% or more, and more preferably 70% or more with respect to the total number of amino acid residues contained therein.

In the major dragline silk, the REP1 corresponds to a crystal region in a fiber where a crystal β sheet is formed, and the REP2 corresponds to an amorphous region in a fiber where most of the parts lack regular configurations and that has more flexibility. Further, the [REP1-REP2] corresponds to a repetitious region (repetitive sequence) composed of the crystal region and the amorphous region, which is a characteristic sequence of dragline silk proteins.

### Recombinant Silk Fragments

In some embodiments, the recombinant silk protein refers to recombinant spider silk polypeptides, recombinant insect silk polypeptides, or recombinant mussel silk polypeptides. In some embodiments, the recombinant silk protein fragment disclosed herein include recombinant spider silk polypeptides of Araneidae or Araneoids, or recombinant insect silk polypeptides of Bombyx mori. In some embodiments, the recombinant silk protein fragment disclosed herein include recombinant spider silk polypeptides of Araneidae or Araneoids. In some embodiments, the recombinant silk protein fragment disclosed herein include block copolymer having repetitive units derived from natural spider silk polypeptides of Araneidae or Araneoids. In some embodiments, the recombinant silk protein fragment disclosed herein include block copolymer having synthetic repetitive units derived from spider silk polypeptides of Araneidae or Araneoids and non-repetitive units derived from natural repetitive units of spider silk polypeptides of Araneidae or Araneoids.

Recent advances in genetic engineering have provided a route to produce various types of recombinant silk proteins. Recombinant DNA technology has been used to provide a more practical source of silk proteins. As used herein “recombinant silk protein” refers to synthetic proteins produced heterologously in prokaryotic or eukaryotic expression systems using genetic engineering methods.

Various methods for synthesizing recombinant silk peptides are known and have been described by Ausubel et al., Current Protocols in Molecular Biology § 8 (John Wiley & Sons 1987, (1990)), incorporated herein by reference. A gram-negative, rod-shaped bacterium E. coli is a well-established host for industrial scale production of proteins. Therefore, the majority of recombinant silks have been produced in E. coli. E. coli which is easy to manipulate, has a short generation time, is relatively low cost and can be scaled up for larger amounts protein production.

The recombinant silk proteins can be produced by transformed prokaryotic or eukaryotic systems containing the cDNA coding for a silk protein, for a fragment of this protein or for an analog of such a protein. The recombinant DNA approach enables the production of recombinant silks with programmed sequences, secondary structures, architectures and precise molecular weight. There are four main steps in the process: (i) design and assembly of synthetic silk-like genes into genetic ‘cassettes’, (ii) insertion of this segment into a DNA recombinant vector, (iii) transformation of this recombinant DNA molecule into a host cell and (iv) expression and purification of the selected clones.

The term “recombinant vectors”, as used herein, includes any vectors known to the skilled person including plasmid vectors, cosmid vectors, phage vectors such as lambda phage, viral vectors such as adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, or plant) or in in vitro expression systems. Cloning vectors are generally used to engineer and amplify a certain desired DNA fragment and may lack functional sequences needed for expression of the desired DNA fragments.

The prokaryotic systems include Gram-negative bacteria or Gram-positive bacteria. The prokaryotic expression vectors can include an origin of replication which can be recognized by the host organism, a homologous or heterologous promoter which is functional in the said host, the DNA sequence coding for the spider silk protein, for a fragment of this protein or for an analogous protein. Nonlimiting examples of prokaryotic expression organisms are Escherichia coli, Bacillus subtilis, Bacillus megaterium, Corynebacterium glutamicum, Anabaena, Caulobacter, Gluconobacter, Rhodobacter, Pseudomonas, Para coccus, Bacillus (e.g. Bacillus subtilis) Brevibacterium, Corynebacterium, Rhizobium (Sinorhizobium), Flavobacterium, Klebsiella, Enterobacter, Lactobacillus, Lactococcus, Methylobacterium, Propionibacterium, Staphylococcus or Streptomyces cells.

The eukaryotic systems include yeasts and insect, mammalian or plant cells. In this case, the expression vectors can include a yeast plasmid origin of replication or an autonomous replication sequence, a promoter, a DNA sequence coding for a spider silk protein, for a fragment or for an analogous protein, a polyadenylation sequence, a transcription termination site and, lastly, a selection gene. Nonlimiting examples of eukaryotic expression organisms include yeasts, such as Saccharomyces cerevisiae, Pichia pastoris, basidiosporogenous, ascosporogenous, filamentous fungi, such as Aspergillus niger, Aspergillus oryzae, Aspergillus nidulans, Trichoderma reesei, Acremonium chrysogenum, Candida, Hansenula, Kluyveromyces, Saccharomyces (e.g. Saccharomyces cerevisiae), Schizosaccharomyces, Pichia (e.g. Pichia pastoris) or Yarrowia cells etc., mammalian cells, such as HeLa cells, COS cells, CHO cells etc., insect cells, such as Sf9 cells, MEL cells, etc., “insect host cells” such as Spodoptera frugiperda or Trichoplusia ni cells. SF9 cells, SF-21 cells or High-Five cells, wherein SF-9 and SF-21 are ovarian cells from Spodoptera frugiperda, and High-Five cells are egg cells from Trichoplusia ni., “plant host cells”, such as tobacco, potato or pea cells.

A variety of heterologous host systems have been explored to produce different types of recombinant silks. Recombinant partial spidroins as well as engineered silks have been cloned and expressed in bacteria (Escherichia coli), yeast (Pichia pastoris), insects (silkworm larvae), plants (tobacco, soybean, potato, Arabidopsis), mammalian cell lines (BHT/hamster) and transgenic animals (mice, goats). Most of the silk proteins are produced with an N- or C-terminal His-tags to make purification simple and produce enough amounts of the protein.

In some embodiments, the host suitable for expressing the recombinant spider silk protein using heterogeneous system may include transgenic animals and plants. In some embodiments, the host suitable for expressing the recombinant spider silk protein using heterogeneous system comprises bacteria, yeasts, mammalian cell lines. In some embodiments, the host suitable for expressing the recombinant spider silk protein using heterogeneous system comprises E coli. In some embodiments, the host suitable for expressing the recombinant spider silk protein using heterogeneous system comprises transgenic B. mori silkworm generated using genome editing technologies (e.g., CRISPR).

The recombinant silk protein in this disclosure comprises synthetic proteins which are based on repeat units of natural silk proteins. Besides the synthetic repetitive silk protein sequences, these can additionally comprise one or more natural nonrepetitive silk protein sequences.

In some embodiments, “recombinant silk protein” refers to recombinant silkworm silk protein or fragments thereof. The recombinant production of silk fibroin and silk sericin has been reported. A variety of hosts are used for the production including E. coli, Saccharomyces cerevisiae, Pseudomonas sp., Rhodopseudomonas sp., Bacillus sp., and Strepomyces. See EP 0230702, which is incorporate by reference herein by its entirety.

Provided herein also include design and biological-synthesis of silk fibroin protein-like multiblock polymer comprising GAGAGX (SEO ID NO:1) hexapeptide (X is A, Y, V or S) derived from the repetitive domain of B. mori silk heavy chain (H chain).

In some embodiments, this disclosure provides silk protein-like multiblock polymers derived from the repetitive domain of B. mori silk heavy chain (H chain) comprising the GAGAGS (SEQ ID NO:2) hexapeptide repeating units. The GAGAGS (SEQ ID NO:2) hexapeptide is the core unit of H-chain and plays an important role in the formation of crystalline domains. The silk protein-like multiblock polymers containing the GAGAGS (SEQ ID NO:2) hexapeptide repeating units spontaneously aggregate into β-sheet structures, similar to natural silk fibroin protein, where in the silk protein-like multiblock polymers having any weight average molecular weight described herein.

In some embodiments, this disclosure provides silk-peptide like multiblock copolymers composed of the GAGAGS (SEQ ID NO:2) hexapeptide repetitive fragment derived from H chain of B. mori silk heavy chain and mammalian elastin VPGVG (SEQ ID NO:3) motif produced by E. coli. In some embodiments, this disclosure provides fusion silk fibroin proteins composed of the GAGAGS (SEQ ID NO:2) hexapeptide repetitive fragment derived from H chain of B. mori silk heavy chain and GVGVP (SEQ ID NO:4) produced by E. coli, where in the silk protein-like multiblock polymers having any weight average molecular weight described herein.

In some embodiments, this disclosure provides B. mori silkworm recombinant proteins composed of the (GAGAGS)₁₆ (SEQ ID NO:5) repetitive fragment. In some embodiments, this disclosure provides recombinant proteins composed of the (GAGAGS)₁₆ (SEQ ID NO:5) repetitive fragment and the non-repetitive (GAGAGS)₁₆-F-COOH, (GAGAGS)₁₆-F-F-COOH, (GAGAGS)₁₆-F-F-F-COOH, (GAGAGS)₁₆-F-F-F-F-COOH, (GAGAGS)₁₆-F-F-F-F-F-F-F-F-COOH, (GAGAGS)₁₆-F-F-F-F-F-F-F-F-F-F-F-F-COOH produced by E. coli, where F has the following amino acid sequence SGFGPVANGGSGEASSESDFGSSGFGPVANASSGEASSESDFAG (SEQ ID NO:6), and where in the silk protein-like multiblock polymers having any weight average molecular weight described herein.

In some embodiments, “recombinant silk protein” refers to recombinant spider silk protein or fragments thereof. The productions of recombinant spider silk proteins based on a partial cDNA clone have been reported. The recombinant spider silk proteins produced as such comprise a portion of the repetitive sequence derived from a dragline spider silk protein, Spidroin 1, from the spider Nephila clavipes. see Xu et al. (Proc. Natl. Acad. Sci. U.S.A., 87:7120-7124 (1990). cDNA clone encoding a portion of the repeating sequence of a second fibroin protein, Spidroin 2, from dragline silk of Nephila clavipes and the recombinant synthesis thereof is described in J. Biol. Chem., 1992, volume 267, pp. 19320-19324. The recombinant synthesis of spider silk proteins including protein fragments and variants of Nephila clavipes from transformed E. coli is described in U.S. Pat. Nos. 5,728,810 and 5,989,894. cDNA clones encoding minor ampullate spider silk proteins and the expression thereof is described in U.S. Pat. Nos. 5,733,771 and 5,756,677. cDNA clone encoding the flagelliform silk protein from an orb-web spinning spider is described in U.S. Pat. No. 5,994,099. U.S. Pat. No. 6,268,169 describes the recombinant synthesis of spider silk like proteins derived from the repeating peptide sequence found in the natural spider dragline of Nephila clavipes by E. coli, Bacillus subtilis, and Pichia pastoris recombinant expression systems. WO 03/020916 describes the cDNA clone encoding and recombinant production of spider spider silk proteins having repeative sequences derived from the major ampullate glands of Nephila madagascariensis, Nephila senegalensis, Tetragnatha kauaiensis, Tetragnatha versicolor, Argiope aurantia, Argiope trifasciata, Gasteracantha mammosa, and Latrodectus geometricus, the flagelliform glands of Argiope trifasciata, the ampullate glands of Dolomedes tenebrosus, two sets of silk glands from Plectreurys tristis, and the silk glands of the mygalomorph Euagrus chisoseus. Each of the above reference is incorporated herein by reference in its entirety.

In some embodiments, the recombinant spider silk protein is a hybrid protein of a spider silk protein and an insect silk protein, a spider silk protein and collagen, a spider silk protein and resilin, or a spider silk protein and keratin. The spider silk repetitive unit comprises or consists of an amino acid sequence of a region that comprises or consists of at least one peptide motif that repetitively occurs within a naturally occurring major ampullate gland polypeptide, such as a dragline spider silk polypeptide, a minor ampullate gland polypeptide, a flagelliform polypeptide, an aggregate spider silk polypeptide, an aciniform spider silk polypeptide or a pyriform spider silk polypeptide.

In some embodiments, the recombinant spider silk protein in this disclosure comprises synthetic spider silk proteins derived from repetitive units of natural spider silk proteins, consensus sequence, and optionally one or more natural non-repetitive spider silk protein sequences. The repeated units of natural spider silk polypeptide may include dragline spider silk polypeptides or flagelliform spider silk polypeptides of Araneidae or Araneoids.

As used herein, the spider silk “repetitive unit” comprises or consists of at least one peptide motif that repetitively occurs within a naturally occurring major ampullate gland polypeptide, such as a dragline spider silk polypeptide, a minor ampullate gland polypeptide, a flagelliform polypeptide, an aggregate spider silk polypeptide, an aciniform spider silk polypeptide or a pyriform spider silk polypeptide. A “repetitive unit” refers to a region which corresponds in amino acid sequence to a region that comprises or consists of at least one peptide motif (e.g. AAAAAA) (SEQ ID NO:21) or GPGQQ (SEQ ID NO:16)) that repetitively occurs within a naturally occurring silk polypeptide (e.g. MaSpI, ADF-3, ADF-4, or Flag) (i.e. identical amino acid sequence) or to an amino acid sequence substantially similar thereto (i.e. variational amino acid sequence). A “repetitive unit” having an amino acid sequence which is “substantially similar” to a corresponding amino acid sequence within a naturally occurring silk polypeptide (i.e. wild-type repetitive unit) is also similar with respect to its properties, e.g. a silk protein comprising the “substantially similar repetitive unit” is still insoluble and retains its insolubility. A “repetitive unit” having an amino acid sequence which is “identical” to the amino acid sequence of a naturally occurring silk polypeptide, for example, can be a portion of a silk polypeptide corresponding to one or more peptide motifs of MaSpI, MaSpII, ADF-3 and/or ADF-4. A “repetitive unit” having an amino acid sequence which is “substantially similar” to the amino acid sequence of a naturally occurring silk polypeptide, for example, can be a portion of a silk polypeptide corresponding to one or more peptide motifs of MaSpI, MaSpII, ADF-3 and/or ADF-4, but having one or more amino acid substitution at specific amino acid positions.

As used herein, the term “consensus peptide sequence” refers to an amino acid sequence which contains amino acids which frequently occur in a certain position (e.g., “G”) and wherein, other amino acids which are not further determined are replaced by the place holder “X”. In some embodiments, the consensus sequence is at least one of (i) GPGXX (SEQ ID NO:7), wherein X is an amino acid selected from A, S, G, Y, P and Q; (ii) GGX, wherein X is an amino acid selected from Y, P, R, S, A, T, N and Q, preferably Y, P and Q; (iii) Ax, wherein x is an integer from 5 to 10.

The consensus peptide sequences GPGXX (SEQ ID NO:7) and GGX, i.e., glycine rich motifs, provide flexibility to the silk polypeptide and thus, to the thread formed from the silk protein containing said motifs. In detail, the iterated GPGXX (SEQ ID NO:7) motif forms turn spiral structures, which imparts elasticity to the silk polypeptide. Major ampullate and flagelliform silks both have a GPGXX (SEQ ID NO:7) motif. The iterated GGX motif is associated with a helical structure having three amino acids per turn and is found in most spider silks. The GGX motif may provide additional elastic properties to the silk. The iterated polyalanine Ax (peptide) motif forms a crystalline β-sheet structure that provides strength to the silk polypeptide, as described for example in WO 03/057727.

In some embodiments, the recombinant spider silk protein in this disclosure comprises two identical repetitive units each comprising at least one, preferably one, amino acid sequence selected from the group consisting of: GGRPSDTYG (SEQ ID NO:8) and GGRPSSSYG (SEQ ID NO:9) derived from Resilin. Resilin is an elastomeric protein found in most arthropods that provides low stiffness and high strength.

As used herein, “non-repetitive units” refers to an amino acid sequence which is “substantially similar” to a corresponding non-repetitive (carboxy terminal) amino acid sequence within a naturally occurring dragline polypeptide (i.e. wild-type non-repetitive (carboxy terminal) unit), preferably within ADF-3, ADF-4, NR3, NR4 of the spider Araneus diadematus, which is also described in U.S. Pat. No. 9,217,017, which is incorporated by reference herein in its entirety, C16 peptide (spider silk protein eADF4, molecular weight of 47.7 kDa, AMSilk) comprising the 16 repeats of the sequence GSSAAAAAAAASGPGGYGPENQGPSGPGGYGPGGP (SEQ ID NO:10), an amino acid sequence adapted from the natural sequence of ADF4 from A. diadematus. Non-repetitive ADF-4 and variants thereof display efficient assembly behavior.

Among the synthetic spider silk proteins, the recombinant silk protein in this disclosure comprises in some embodiments the C16-protein having the polypeptide sequence SEQ ID NO: 1 as described in U.S. Pat. No. 8,288,512, which is incorporated by reference herein in its entirety. Besides the polypeptide sequence shown in SEQ ID NO: 1 as described in U.S. Pat. No. 8,288,512, particularly functional equivalents, functional derivatives and salts of this sequence are also included.

As used herein, “functional equivalents” refers to mutant which, in at least one sequence position of the abovementioned amino acid sequences, have an amino acid other than that specifically mentioned.

In some embodiments, the recombinant spider silk protein in this disclosure comprises, in an effective amount, at least one natural or recombinant silk protein including spider silk protein, corresponding to Spidroin major 1 described by Xu et al., PNAS, USA, 87, 7120, (1990), Spidroin major 2 described by Hinman and Lewis, J. Biol. Chem., 267, 19320, (1922), recombinant spider silk protein as described in U.S. Patent Application No. 2016/0222174 and U.S. Pat. Nos. 9,051,453, 9,617,315, 9,689,089, 8,173,772, 8,642,734, 8,367,803 8,097,583, 8,030,024, 7,754,851, 7,148,039, 7,060,260, or alternatively the minor Spidroins described in patent application WO 95/25165. Each of the above-cited references is incorporated herein by reference in its entirety. Additional recombinant spider silk proteins suitable for the recombinant RSPF of this disclosure include ADF3 and ADF4 from the “Major Ampullate” gland of Araneus diadematus.

Recombinant silk is also described in other patents and patent applications, incorporated by reference herein: US 2004590196, U.S. Pat. No. 7,754,851, US 2007654470, U.S. Pat. No. 7,951,908, US 2010785960, U.S. Pat. No. 8,034,897, US 20090263430, US 2008226854, US 20090123967, US 2005712095, US 2007991037, US 20090162896, US 200885266, U.S. Pat. No. 8,372,436, US 2007989907, US 2009267596, US 2010319542, US 2009265344, US 2012684607, US 2004583227, U.S. Pat. No. 8,030,024, US 2006643569, U.S. Pat. No. 7,868,146, US 2007991916, U.S. Pat. No. 8,097,583, US 2006643200, U.S. Pat. Nos. 8,729,238, 8,877,903, US 20190062557, US 20160280960, US 20110201783, US 2008991916, US 2011986662, US 2012697729, US 20150328363, U.S. Pat. No. 9,034,816, US 20130172478, U.S. Pat. No. 9,217,017, US 20170202995, U.S. Pat. No. 8,721,991, US 2008227498, U.S. Pat. Nos. 9,233,067, 8,288,512, US 2008161364, U.S. Pat. No. 7,148,039, U.S. Ser. No. 19/992,47806, US 2001861597, US 2004887100, U.S. Pat. Nos. 9,481,719, 8,765,688, US 200880705, US 2010809102, U.S. Pat. No. 8,367,803, US 2010664902, U.S. Pat. No. 7,569,660, U.S. Ser. No. 19/991,38833, US 2000591632, US 20120065126, US 20100278882, US 2008161352, US 20100015070, US 2009513709, US 20090194317, US 2004559286, US 200589551, US 2008187824, US 20050266242, US 20050227322, and US 20044418. Recombinant silk is also described in other patents and patent applications, incorporated by reference herein: US 20190062557, US 20150284565, US 20130225476, US 20130172478, US 20130136779, US 20130109762, US 20120252294, US 20110230911, US 20110201783, US 20100298877, U.S. Pat. Nos. 10,478,520, 10,253,213, 10,072,152, 9,233,067, 9,217,017, 9,034,816, 8,877,903, 8,729,238, 8,721,991, 8,097,583, 8,034,897, 8,030,024, 7,951,908, 7,868,146, and 7,754,851.

In some embodiments, the recombinant spider silk protein in this disclosure comprises or consists of 2 to 80 repetitive units, each independently selected from GPGXX (SEQ ID NO: 7), GGX and Ax as defined herein.

In some embodiments, the recombinant spider silk protein in this disclosure comprises or consists of repetitive units each independently selected from selected from the group consisting of (SEQ ID NO:11-SEQ ID NO:26) GPGAS, GPGSG, GPGGY, GPGGP, GPGGA, GPGQQ, GPGGG, GPGQG, GPGGS, GGY, GGP, GGA, GGR, GGS, GGT, GGN, GGQ, AAAAA, AAAAAA, AAAAAAA, AAAAAAAA, AAAAAAAAA, AAAAAAAAAA, GGRPSDTYG and GGRPSSSYG (SEQ ID NO: 27), (i) GPYGPGASAAAAAAGGYGPGSGQQ (SEQ ID NO: 28), (ii) GSSAAAAAAAASGPGGYGPENQGPSGPGGYGPGGP (SEQ ID NO:29), (iii) GPGQQGPGQQGPGQQGPGQQ (SEQ ID NO:30) (iv) GPGGAGGPYGPGGAGGPYGPGGAGGPY (SEQ ID NO:31), (v) GGTTIIEDLDITIDGADGPITISEELTI (SEQ ID NO:32), (vi) PGSSAAAAAAAASGPGQGQGQGQGQGGRPSDTYG (SEQ ID NO:33), (vii) SAAAAAAAAGPGGGNGGRPSDTYGAPGGGNGGRPSSSYG (SEQ ID NO:34), (viii) GGAGGAGGAGGSGGAGGS (SEQ ID NO:35), (ix) GPGGAGPGGYGPGGSGPGGYGPGGSGPGGY (SEQ ID NO:36), (x) GPYGPGASAAAAAAGGYGPGCGQQ (SEQ ID NO:37), (xi) GPYGPGASAAAAAAGGYGPGKGQQ (SEQ ID NO:38), (xii) GSSAAAAAAAASGPGGYGPENQGPCGPGGYGPGGP (SEQ ID NO:39), (xiii) GSSAAAAAAAASGPGGYGPKNQGPSGPGGYGPGGP (SEQ ID NO:40), (xiv) GSSAAAAAAAASGPGGYGPKNQGPSGPGGYGPGGP (SEQ ID NO:40), or variants thereof as described in U.S. Pat. No. 8,877,903, for example, a synthetic spider peptide having sequential order of GPGAS (SEQ ID NO:11), GGY, GPGSG (SEQ ID NO:12) in the peptide chain, or sequential order of AAAAAAAA (SEQ ID NO:23), GPGGY (SEQ ID NO:13), GPGGP SEQ ID NO:14 in the peptide chain, sequential order of AAAAAAAA (SEQ ID NO:23), GPGQG (SEQ ID NO:18), GGR in the peptide chain.

In some embodiments, this disclosure provides silk protein-like multiblock peptides that imitate the repeating units of amino acids derived from natural spider silk proteins such as Spidoin major 1 domain, Spidoin major 2 domain or Spidoin minor 1 domain and the profile of variation between the repeating units without modifying their three-dimensional conformation, wherein these silk protein-like multiblock peptides comprise a repeating unit of amino acids corresponding to one of the sequences (I), (II), (III) and/or (IV) below.
[(XGG)_{w}(XGA)(GXG)ₓ(AGA)_{y}(G)_{z}AG]ₚ Formula (I) in which: X corresponds to tyrosine or to glutamine, w is an integer equal to 2 or 3, x is an integer from 1 to 3, y is an integer from 5 to 7, z is an integer equal to 1 or 2, and p is an integer and having any weight average molecular weight described herein, and/or
[(GPG₂YGPGQ₂)ₐ(X′)₂S(A)_{b}]ₚ Formula (II) in which: X′ corresponds to the amino acid sequence GPS or GPG, a is equal to 2 or 3, b is an integer from 7 to 10, and p is an integer and having any weight average molecular weight described herein, and/or
[(GR)(GA)ₗ(A)ₘ(GGX)ₙ(GA)ₗ(A)ₘ]ₚ Formula (III) and/or [(GGX″)ₙ(GA)ₘ(A)ₗ]ₚ Formula (IV) in which: X″ corresponds to tyrosine, glutamine or alanine, l is an integer from 1 to 6, m is an integer from 0 to 4, n is an integer from 1 to 4, and p is an integer.

In some embodiments, the recombinant spider silk protein or an analog of a spider silk protein comprising an amino acid repeating unit of sequence (V):
[(Xaa Gly Gly)_{w}(Xaa Gly Ala)(Gly Xaa Gly)ₓ(Ala Gly Ala)_{y}(Gly)_{z}Ala Gly]ₚFormula (V), wherein Xaa is tyrosine or glutamine, w is an integer equal to 2 or 3, x is an integer from 1 to 3, y is an integer from 5 to 7, z is an integer equal to 1 or 2, and p is an integer.

In some embodiments, the recombinant spider silk protein in this disclosure is selected from the group consisting of ADF-3 or variants thereof, ADF-4 or variants thereof, MaSpI or variants thereof, MaSpII or variants thereof as described in U.S. Pat. No. 9,217,017.

In some embodiments, this disclosure provides water soluble recombinant spider silk proteins produced in mammalian cells. The solubility of the spider silk proteins produced in mammalian cells was attributed to the presence of the COOH-terminus in these proteins, which makes them more hydrophilic. These COOH-terminal amino acids are absent in spider silk proteins expressed in microbial hosts.

In some embodiments, the recombinant spider silk protein in this disclosure comprises water soluble recombinant spider silk protein C16 modified with an amino or carboxyl terminal selected from the amino acid sequences consisting of: GCGGGGGG (SEQ ID NO:41), GKGGGGGG (SEQ ID NO:42), GCGGSGGGGSGGGG (SEQ ID NO:43), GKGGGGGGSGGGG (SEQ ID NO:44), and GCGGGGGGSGGGG (SEQ ID NO:45). In some embodiments, the recombinant spider silk protein in this disclosure comprises C₁₆NR4, C₃₂NR4, C16, C32, NR4C₁₆NR4, NR4C₃₂NR4, NR3C₁₆NR3, or NR3C₃₂NR3 such that the molecular weight of the protein ranges as described herein.

In some embodiments, the recombinant spider silk protein in this disclosure comprises recombinant spider silk protein having a synthetic repetitive peptide segments and an amino acid sequence adapted from the natural sequence of ADF4 from A. diadematus as described in U.S. Pat. No. 8,877,903. In some embodiments, the RSPF in this disclosure comprises the recombinant spider silk proteins having repeating peptide units derived from natural spider silk proteins such as Spidroin major 1 domain, Spidroin major 2 domain or Spidroin minor 1 domain, wherein the repeating tide sequence is

(SEQ ID NO: 46)
GSSAAAAAAAASGPGQGQGQGQGQGGRPSDTYG
or
(SEQ ID NO: 47)
SAAAAAAAAGPGGGNGGRPSDTYGAPGGGNGGRPSSSYG,

as described in U.S. Pat. No. 8,367,803, which is incorporated by reference herein in its entirety.

In some embodiments, this disclosure provides recombinant spider proteins composed of the GPGGAGPGGYGPGGSGPGGYGPGGSGPGGY (SEQ ID NO:48) repetitive fragment and having a molecular weight as described herein.

As used herein, the term “recombinant silk” refers to recombinant spider and/or silkworm silk protein or fragments thereof. In an embodiment, the spider silk protein is selected from the group consisting of swathing silk (Achniform gland silk), egg sac silk (Cylindriform gland silk), egg case silk (Tubuliform silk), non-sticky dragline silk (Ampullate gland silk), attaching thread silk (Pyriform gland silk), sticky silk core fibers (Flagelliform gland silk), and sticky silk outer fibers (Aggregate gland silk). For example, recombinant spider silk protein, as described herein, includes the proteins described in U.S. Patent Application No. 2016/0222174 and U.S. Pat. Nos. 9,051,453, 9,617,315, 9,689,089, 8,173,772, and 8,642,734.

Some organisms make multiple silk fibers with unique sequences, structural elements, and mechanical properties. For example, orb weaving spiders have six unique types of glands that produce different silk polypeptide sequences that are polymerized into fibers tailored to fit an environmental or lifecycle niche. The fibers are named for the gland they originate from and the polypeptides are labeled with the gland abbreviation (e.g. “Ma”) and “Sp” for spidroin (short for spider fibroin). In orb weavers, these types include Major Ampullate (MaSp, also called dragline), Minor Ampullate (MiSp), Flagelliform (Flag), Aciniform (AcSp), Tubuliform (TuSp), and Pyriform (PySp). This combination of polypeptide sequences across fiber types, domains, and variation amongst different genus and species of organisms leads to a vast array of potential properties that can be harnessed by commercial production of the recombinant fibers. To date, the vast majority of the work with recombinant silks has focused on the Major Ampullate Spidroins (MaSp).

Aciniform (AcSp) silks tend to have high toughness, a result of moderately high strength coupled with moderately high extensibility. AcSp silks are characterized by large block (“ensemble repeat”) sizes that often incorporate motifs of poly serine and GPX. Tubuliform (TuSp or Cylindrical) silks tend to have large diameters, with modest strength and high extensibility. TuSp silks are characterized by their poly serine and poly threonine content, and short tracts of poly alanine. Major Ampullate (MaSp) silks tend to have high strength and modest extensibility. MaSp silks can be one of two subtypes: MaSpI and MaSp2. MaSpI silks are generally less extensible than MaSp2 silks, and are characterized by poly alanine, GX, and GGX motifs. MaSp2 silks are characterized by poly alanine, GGX, and GPX motifs. Minor Ampullate (MiSp) silks tend to have modest strength and modest extensibility. MiSp silks are characterized by GGX, GA, and poly A motifs, and often contain spacer elements of approximately 100 amino acids. Flagelliform (Flag) silks tend to have very high extensibility and modest strength. Flag silks are usually characterized by GPG, GGX, and short spacer motifs.

Silk polypeptides are characteristically composed of a repeat domain (REP) flanked by non-repetitive regions (e.g., C-terminal and N-terminal domains). In an embodiment, both the C-terminal and N-terminal domains are between 75-350 amino acids in length. The repeat domain exhibits a hierarchical architecture. The repeat domain comprises a series of blocks (also called repeat units). The blocks are repeated, sometimes perfectly and sometimes imperfectly (making up a quasi-repeat domain), throughout the silk repeat domain. The length and composition of blocks varies among different silk types and across different species. Table 1 of U.S. Published Application No. 2016/0222174, the entirety of which is incorporated herein, lists examples of block sequences from selected species and silk types, with further examples presented in Rising, A. et al., Spider silk proteins: recent advances in recombinant production, structure-function relationships and biomedical applications, Cell Mol. Life Sci., 68:2, pg 169-184 (2011); and Gatesy, J. et al., Extreme diversity, conservation, and convergence of spider silk fibroin sequences, Science, 291:5513, pg. 2603-2605 (2001). In some cases, blocks may be arranged in a regular pattern, forming larger macro-repeats that appear multiple times (usually 2-8) in the repeat domain of the silk sequence. Repeated blocks inside a repeat domain or macro-repeat, and repeated macro-repeats within the repeat domain, may be separated by spacing elements.

The construction of certain spider silk block copolymer polypeptides from the blocks and/or macro-repeat domains, according to certain embodiments of the disclosure, is illustrated in U.S. Published Patent Application No. 2016/0222174.

The recombinant block copolymer polypeptides based on spider silk sequences produced by gene expression in a recombinant prokaryotic or eukaryotic system can be purified according to methods known in the art. In a preferred embodiment, a commercially available expression/secretion system can be used, whereby the recombinant polypeptide is expressed and thereafter secreted from the host cell, to be easily purified from the surrounding medium. If expression/secretion vectors are not used, an alternative approach involves purifying the recombinant block copolymer polypeptide from cell lysates (remains of cells following disruption of cellular integrity) derived from prokaryotic or eukaryotic cells in which a polypeptide was expressed. Methods for generation of such cell lysates are known to those of skill in the art. In some embodiments, recombinant block copolymer polypeptides are isolated from cell culture supernatant.

Recombinant block copolymer polypeptide may be purified by affinity separation, such as by immunological interaction with antibodies that bind specifically to the recombinant polypeptide or nickel columns for isolation of recombinant polypeptides tagged with 6-8 histidine residues at their N-terminus or C-terminus Alternative tags may comprise the FLAG epitope or the hemagglutinin epitope. Such methods are commonly used by skilled practitioners.

A solution of such polypeptides (i.e., recombinant silk protein) may then be prepared and used as described herein.

In another embodiment, recombinant silk protein may be prepared according to the methods described in U.S. Pat. No. 8,642,734, the entirety of which is incorporated herein, and used as described herein.

In an embodiment, a recombinant spider silk protein is provided. The spider silk protein typically consists of from 170 to 760 amino acid residues, such as from 170 to 600 amino acid residues, preferably from 280 to 600 amino acid residues, such as from 300 to 400 amino acid residues, more preferably from 340 to 380 amino acid residues. The small size is advantageous because longer spider silk proteins tend to form amorphous aggregates, which require use of harsh solvents for solubilization and polymerization. The recombinant spider silk protein may contain more than 760 residues, in particular in cases where the spider silk protein contains more than two fragments derived from the N-terminal part of a spider silk protein, The spider silk protein comprises an N-terminal fragment consisting of at least one fragment (NT) derived from the corresponding part of a spider silk protein, and a repetitive fragment (REP) derived from the corresponding internal fragment of a spider silk protein. Optionally, the spider silk protein comprises a C-terminal fragment (CT) derived from the corresponding fragment of a spider silk protein. The spider silk protein comprises typically a single fragment (NT) derived from the N-terminal part of a spider silk protein, but in preferred embodiments, the N-terminal fragment include at least two, such as two fragments (NT) derived from the N-terminal part of a spider silk protein. Thus, the spidroin can schematically be represented by the formula NTₘ-REP, and alternatively NTₘ-REP-CT, where m is an integer that is 1 or higher, such as 2 or higher, preferably in the ranges of 1-2, 1-4, 1-6, 2-4 or 2-6. Preferred spidroins can schematically be represented by the formulas NT₂-REP or NT-REP, and alternatively NT₂-REP-CT or NT-REP-CT. The protein fragments are covalently coupled, typically via a peptide bond. In one embodiment, the spider silk protein consists of the NT fragment(s) coupled to the REP fragment, which REP fragment is optionally coupled to the CT fragment.

In one embodiment, the first step of the method of producing polymers of an isolated spider silk protein involves expression of a polynucleic acid molecule which encodes the spider silk protein in a suitable host, such as Escherichia coli. The thus obtained protein is isolated using standard procedures. Optionally, lipopolysaccharides and other pyrogens are actively removed at this stage.

In the second step of the method of producing polymers of an isolated spider silk protein, a solution of the spider silk protein in a liquid medium is provided. By the terms “soluble” and “in solution” is meant that the protein is not visibly aggregated and does not precipitate from the solvent at 60,000×g. The liquid medium can be any suitable medium, such as an aqueous medium, preferably a physiological medium, typically a buffered aqueous medium, such as a 10-50 mM Tris-HCl buffer or phosphate buffer. The liquid medium has a pH of 6.4 or higher and/or an ion composition that prevents polymerization of the spider silk protein. That is, the liquid medium has either a pH of 6.4 or higher or an ion composition that prevents polymerization of the spider silk protein, or both.

Ion compositions that prevent polymerization of the spider silk protein can readily be prepared by the skilled person utilizing the methods disclosed herein. A preferred ion composition that prevents polymerization of the spider silk protein has an ionic strength of more than 300 mM. Specific examples of ion compositions that prevent polymerization of the spider silk protein include above 300 mM NaCl, 100 mM phosphate and combinations of these ions having desired preventive effect on the polymerization of the spider silk protein, e.g. a combination of 10 mM phosphate and 300 mM NaCl.

The presence of an NT fragment improves the stability of the solution and prevents polymer formation under these conditions. This can be advantageous when immediate polymerization may be undesirable, e.g. during protein purification, in preparation of large batches, or when other conditions need to be optimized. It is preferred that the pH of the liquid medium is adjusted to 6.7 or higher, such as 7.0 or higher, or even 8.0 or higher, such as up to 10.5, to achieve high solubility of the spider silk protein. It can also be advantageous that the pH of the liquid medium is adjusted to the range of 6.4-6.8, which provides sufficient solubility of the spider silk protein but facilitates subsequent pH adjustment to 6.3 or lower.

In the third step, the properties of the liquid medium are adjusted to a pH of 6.3 or lower and ion composition that allows polymerization. That is, if the liquid medium wherein the spider silk protein is dissolved has a pH of 6.4 or higher, the pH is decreased to 6.3 or lower. The skilled person is well aware of various ways of achieving this, typically involving addition of a strong or weak acid. If the liquid medium wherein the spider silk protein is dissolved has an ion composition that prevents polymerization, the ion composition is changed so as to allow polymerization. The skilled person is well aware of various ways of achieving this, e.g. dilution, dialysis or gel filtration. If required, this step involves both decreasing the pH of the liquid medium to 6.3 or lower and changing the ion composition so as to allow polymerization. It is preferred that the pH of the liquid medium is adjusted to 6.2 or lower, such as 6.0 or lower. In particular, it may be advantageous from a practical point of view to limit the pH drop from 6.4 or 6.4-6.8 in the preceding step to 6.3 or 6.0-6.3, e.g. 6.2 in this step. In a preferred embodiment, the pH of the liquid medium of this step is 3 or higher, such as 4.2 or higher. The resulting pH range, e.g. 4.2-6.3 promotes rapid polymerization,

In the fourth step, the spider silk protein is allowed to polymerize in the liquid medium having pH of 6.3 or lower and an ion composition that allows polymerization of the spider silk protein. Although the presence of the NT fragment improves solubility of the spider silk protein at a pH of 6.4 or higher and/or an ion composition that prevents polymerization of the spider silk protein, it accelerates polymer formation at a pH of 6.3 or lower when the ion composition allows polymerization of the spider silk protein. The resulting polymers are preferably solid and macroscopic, and they are formed in the liquid medium having a pH of 6.3 or lower and an ion composition that allows polymerization of the spider silk protein. In a preferred embodiment, the pH of the liquid medium of this step is 3 or higher, such as 4.2 or higher. The resulting pH range, e.g. 4.2-6.3 promotes rapid polymerization, Resulting polymer may be provided at the molecular weights described herein and prepared as a solution form that may be used as necessary for article coatings.

Ion compositions that allow polymerization of the spider silk protein can readily be prepared by the skilled person utilizing the methods disclosed herein. A preferred ion composition that allows polymerization of the spider silk protein has an ionic strength of less than 300 mM. Specific examples of ion compositions that allow polymerization of the spider silk protein include 150 mM NaCl, 10 mM phosphate, 20 mM phosphate and combinations of these ions lacking preventive effect on the polymerization of the spider silk protein, e.g. a combination of 10 mM phosphate or 20 mM phosphate and 150 mM NaCl. It is preferred that the ionic strength of this liquid medium is adjusted to the range of 1-250 mM.

Without desiring to be limited to any specific theory, it is envisaged that the NT fragments have oppositely charged poles, and that environmental changes in pH affects the charge balance on the surface of the protein followed by polymerization, whereas salt inhibits the same event.

At neutral pH, the energetic cost of burying the excess negative charge of the acidic pole may be expected to prevent polymerization. However, as the dimer approaches its isoelectric point at lower pH, attractive electrostatic forces will eventually become dominant, explaining the observed salt and pH-dependent polymerization behavior of NT and NT-containing minispidroins. It is proposed that, in some embodiments, pH-induced NT polymerization, and increased efficiency of fiber assembly of NT-minispidroins, are due to surface electrostatic potential changes, and that clustering of acidic residues at one pole of NT shifts its charge balance such that the polymerization transition occurs at pH values of 6.3 or lower.

In a fifth step, the resulting, preferably solid spider silk protein polymers are isolated from said liquid medium. Optionally, this step involves actively removing lipopolysaccharides and other pyrogens from the spidroin polymers.

Without desiring to be limited to any specific theory, it has been observed that formation of spidroin polymers progresses via formation of water-soluble spidroin dimers. The present disclosure thus also provides a method of producing dimers of an isolated spider silk protein, wherein the first two method steps are as described above. The spider silk proteins are present as dimers in a liquid medium at a pH of 6.4 or higher and/or an ion composition that prevents polymerization of said spider silk protein. The third step involves isolating the dimers obtained in the second step, and optionally removal of lipopolysaccharides and other pyrogens. In a preferred embodiment, the spider silk protein polymer of the disclosure consists of polymerized protein dimers. The present disclosure thus provides a novel use of a spider silk protein, preferably those disclosed herein, for producing dimers of the spider silk protein.

According to another aspect, the disclosure provides a polymer of a spider silk protein as disclosed herein. In an embodiment, the polymer of this protein is obtainable by any one of the methods therefor according to the disclosure. Thus, the disclosure provides various uses of recombinant spider silk protein, preferably those disclosed herein, for producing polymers of the spider silk protein as recombinant silk based coatings. According to one embodiment, the present disclosure provides a novel use of a dimer of a spider silk protein, preferably those disclosed herein, for producing polymers of the isolated spider silk protein as recombinant silk based coatings. In these uses, it is preferred that the polymers are produced in a liquid medium having a pH of 6.3 or lower and an ion composition that allows polymerization of said spider silk protein. In an embodiment, the pH of the liquid medium is 3 or higher, such as 4.2 or higher. The resulting pH range, e.g. 4.2-6.3 promotes rapid polymerization, Using the method(s) of the present disclosure, it is possible to control the polymerization process, and this allows for optimization of parameters for obtaining silk polymers with desirable properties and shapes.

In an embodiment, the recombinant silk proteins described herein, include those described in U.S. Pat. No. 8,642,734, the entirety of which is incorporated by reference.

In another embodiment, the recombinant silk proteins described herein may be prepared according to the methods described in U.S. Pat. No. 9,051,453, the entirety of which is incorporated herein by reference.

An amino acid sequence represented by SEQ ID NO: 1 of U.S. Pat. No. 9,051,453, is identical to an amino acid sequence that is composed of 50 amino acid residues of an amino acid sequence of ADF3 at the C-terminal (NCBI Accession No.: AAC47010, GI: 1263287). An amino acid sequence represented by SEQ ID NO: 2 of U.S. Pat. No. 9,051,453, is identical to an amino acid sequence represented by SEQ ID NO: 1 of U.S. Pat. No. 9,051,453, from which 20 residues have been removed from the C-terminal. An amino acid sequence represented by SEQ ID NO: 3 of U.S. Pat. No. 9,051,453, is identical to an amino acid sequence represented by SEQ ID NO: 1 of U.S. Pat. No. 9,051,453 from which 29 residues have been removed from the C-terminal.

An example of the polypeptide that contains units of the amino acid sequence represented by the formula 1: REP1-REP2 (1) and that has, at a C-terminal, an amino acid sequence represented by any of SEQ ID NOS: 1 to 3 of U.S. Pat. No. 9,051,453 or an amino acid sequence having a homology of 90% or more with the amino acid sequence represented by any of SEQ ID NOS: 1 to 3 of U.S. Pat. No. 9,051,453, is a polypeptide having an amino acid sequence represented by SEQ ID NO: 8 of U.S. Pat. No. 9,051,453, which is incorporated by reference herein in its entirety. The polypeptide having the amino acid sequence represented by SEQ ID NO: 8 of U.S. Pat. No. 9,051,453, is obtained by the following mutation: in an amino acid sequence of ADF3 (NCBI Accession No.: AAC47010, GI: 1263287) to the N-terminal of which has been added an amino acid sequence (SEQ ID NO: 5 of U.S. Pat. No. 9,051,453) composed of a start codon, His 10 tags and an HRV3C Protease (Human rhinovirus 3C Protease) recognition site, 1^{th} to 13^{th} repetitive regions are about doubled and the translation ends at the 1154^{th} amino acid residue. In the polypeptide having the amino acid sequence represented by SEQ ID NO: 8 of U.S. Pat. No. 9,051,453, the C-terminal sequence is identical to the amino acid sequence represented by SEQ ID NO: 3 of U.S. Pat. No. 9,051,453.

Further, the polypeptide that contains units of the amino acid sequence represented by the formula 1: REP1-REP2 (1) and that has, at a C-terminal, an amino acid sequence represented by any of SEQ ID NOS: 1 to 3 of U.S. Pat. No. 9,051,453, or an amino acid sequence having a homology of 90% or more with the amino acid sequence represented by any of SEQ ID NOS: 1 to 3 of U.S. Pat. No. 9,051,453, may be a protein that has an amino acid sequence represented by SEQ ID NO: 8 of U.S. Pat. No. 9,051,453, in which one or a plurality of amino acids have been substituted, deleted, inserted and/or added and that has a repetitious region composed of a crystal region and an amorphous region.

Further, an example of the polypeptide containing two or more units of the amino acid sequence represented by the formula 1: REP1-REP2 (1) is a recombinant protein derived from ADF4 having an amino acid sequence represented by SEQ ID NO: 15 of U.S. Pat. No. 9,051,453, which is incorporated by reference herein in its entirety. The amino acid sequence represented by SEQ ID NO: 15 of U.S. Pat. No. 9,051,453, is an amino acid sequence obtained by adding the amino acid sequence (SEQ ID NO: 5 of U.S. Pat. No. 9,051,453) composed of a start codon, His 10 tags and an HRV3C Protease (Human rhinovirus 3C Protease) recognition site, to the N-terminal of a partial amino acid sequence of ADF4 obtained from the NCBI database (NCBI Accession No.: AAC47011, GI: 1263289). Further, the polypeptide containing two or more units of the amino acid sequence represented by the formula 1: REP1-REP2 (1) may be a polypeptide that has an amino acid sequence represented by SEQ ID NO: 15 of U.S. Pat. No. 9,051,453, in which one or a plurality of amino acids have been substituted, deleted, inserted and/or added and that has a repetitious region composed of a crystal region and an amorphous region. Further, an example of the polypeptide containing two or more units of the amino acid sequence represented by the formula 1: REP1-REP2 (1) is a recombinant protein derived from MaSp2 that has an amino acid sequence represented by SEQ ID NO: 17 of U.S. Pat. No. 9,051,453, which is incorporated by reference here in its entirety. The amino acid sequence represented by SEQ ID NO: 17 of U.S. Pat. No. 9,051,453, is an amino acid sequence obtained by adding the amino acid sequence (SEQ ID NO: 5 of U.S. Pat. No. 9,051,453,) composed of a start codon, His 10 tags and an HRV3C Protease (Human rhinovirus 3C Protease) recognition site, to the N-terminal of a partial sequence of MaSp2 obtained from the NCBI web database (NCBI Accession No.: AAT75313, GI: 50363147). Furthermore, the polypeptide containing two or more units of the amino acid sequence represented by the formula 1: REP1-REP2 (1) may be a polypeptide that has an amino acid sequence represented by SEQ ID NO: 17 of U.S. Pat. No. 9,051,453, in which one or a plurality of amino acids have been substituted, deleted, inserted and/or added and that has a repetitious region composed of a crystal region and an amorphous region.

Examples of the polypeptide derived from flagelliform silk proteins include a polypeptide containing 10 or more units of an amino acid sequence represented by the formula 2: REP3 (2), preferably a polypeptide containing 20 or more units thereof, and more preferably a polypeptide containing 30 or more units thereof. In the case of producing a recombinant protein using a microbe such as Escherichia coli as a host, the molecular weight of the polypeptide derived from flagelliform silk proteins is preferably 500 kDa or less, more preferably 300 kDa or less, and further preferably 200 kDa or less, in terms of productivity.

In the formula (2), the REP 3 indicates an amino acid sequence composed of Gly-Pro-Gly-Gly-X, where X indicates an amino acid selected from the group consisting of Ala, Ser, Tyr and Val.

A major characteristic of the spider silk is that the flagelliform silk does not have a crystal region, but has a repetitious region composed of an amorphous region. Since the major dragline silk and the like have a repetitious region composed of a crystal region and an amorphous region, they are expected to have both high stress and stretchability. Meanwhile, as to the flagelliform silk, although the stress is inferior to that of the major dragline silk, the stretchability is high. The reason for this is considered to be that most of the flagelliform silk is composed of amorphous regions.

An example of the polypeptide containing 10 or more units of the amino acid sequence represented by the formula 2: REP3 (2) is a recombinant protein derived from flagelliform silk proteins having an amino acid sequence represented by SEQ ID NO: 19 of U.S. Pat. No. 9,051,453, which is incorporated by reference herein in its entirety. The amino acid sequence represented by SEQ ID NO: 19 of U.S. Pat. No. 9,051,453, is an amino acid sequence obtained by combining a partial sequence of flagelliform silk protein of Nephila clavipes obtained from the NCBI database (NCBI Accession No.: AAF36090, GI: 7106224), specifically, an amino acid sequence thereof from the 1220^{th}residue to the 1659^{th} residue from the N-terminal that corresponds to repetitive sections and motifs (referred to as a PR1 sequence), with a partial sequence of flagelliform silk protein of Nephila clavipes obtained from the NCBI database (NCBI Accession No.: AAC38847, GI: 2833649), specifically, a C-terminal amino acid sequence thereof from the 816^{th} residue to the 907^{th} residue from the C-terminal, and thereafter adding the amino acid sequence (SEQ ID NO: 5 of U.S. Pat. No. 9,051,453) composed of a start codon, His 10 tags and an HRV3C Protease recognition site, to the N-terminal of the combined sequence. Further, the polypeptide containing 10 or more units of the amino acid sequence represented by the formula 2: REP3 (2) may be a polypeptide that has an amino acid sequence represented by SEQ ID NO: 19 of U.S. Pat. No. 9,051,453, in which one or a plurality of amino acids have been substituted, deleted, inserted and/or added and that has a repetitious region composed of an amorphous region.

The polypeptide can be produced using a host that has been transformed by an expression vector containing a gene encoding a polypeptide. A method for producing a gene is not limited particularly, and it may be produced by amplifying a gene encoding a natural spider silk protein from a cell derived from spiders by a polymerase chain reaction (PCR), etc., and cloning it, or may be synthesized chemically. Also, a method for chemically synthesizing a gene is not limited particularly, and it can be synthesized as follows, for example: based on information of amino acid sequences of natural spider silk proteins obtained from the NCBI web database, etc., oligonucleotides that have been synthesized automatically with AKTA oligopilot plus 10/100 (GE Healthcare Japan Corporation) are linked by PCR, etc. At this time, in order to facilitate the purification and observation of protein, it is possible to synthesize a gene that encodes a protein having an amino acid sequence of the above-described amino acid sequence to the N-terminal of which has been added an amino acid sequence composed of a start codon and His 10 tags.

Examples of the expression vector include a plasmid, a phage, a virus, and the like that can express protein based on a DNA sequence. The plasmid-type expression vector is not limited particularly as long as it allows a target gene to be expressed in a host cell and it can amplify itself. For example, in the case of using Escherichia coli Rosetta (DE3) as a host, a pET22b(+) plasmid vector, a pCold plasmid vector, and the like can be used. Among these, in terms of productivity of protein, it is preferable to use the pET22b(+) plasmid vector. Examples of the host include animal cells, plant cells, microbes, etc.

The polypeptide used in the present disclosure is preferably a polypeptide derived from ADF3, which is one of two principal dragline silk proteins of Araneus diadematus. This polypeptide has advantages of basically having high strength-elongation and toughness and of being synthesized easily.

Accordingly, the recombinant silk protein (e.g., the recombinant spider silk-based protein) used in accordance with the embodiments, articles, and/or methods described herein, may include one or more recombinant silk proteins described above or recited in U.S. Pat. Nos. 8,173,772, 8,278,416, 8,618,255, 8,642,734, 8,691,581, 8,729,235, 9,115,204, 9,157,070, 9,309,299, 9,644,012, 9,708,376, 9,051,453, 9,617,315, 9,968,682, 9,689,089, 9,732,125, 9,856,308, 9,926,348, 10,065,997, 10,316,069, and 10,329,332; and U.S. Patent Publication Nos. 2009/0226969, 2011/0281273, 2012/0041177, 2013/0065278, 2013/0115698, 2013/0316376, 2014/0058066, 2014/0079674, 2014/0245923, 2015/0087046, 2015/0119554, 2015/0141618, 2015/0291673, 2015/0291674, 2015/0239587, 2015/0344542, 2015/0361144, 2015/0374833, 2015/0376247, 2016/0024464, 2017/0066804, 2017/0066805, 2015/0293076, 2016/0222174, 2017/0283474, 2017/0088675, 2019/0135880, 2015/0329587, 2019/0040109, 2019/0135881, 2019/0177363, 2019/0225646, 2019/0233481, 2019/0031842, 2018/0355120, 2019/0186050, 2019/0002644, 2020/0031887, 2018/0273590, 20191/094403, 2019/0031843, 2018/0251501, 2017/0066805, 2018/0127553, 2019/0329526, 2020/0031886, 2018/0080147, 2019/0352349, 2020/0043085, 2019/0144819, 2019/0228449, 2019/0340666, 2020/0000091, 2019/0194710, 2019/0151505, 2018/0265555, 2019/0352330, 2019/0248847, and 2019/0378191, the entirety of which are incorporated herein by reference.

### Silk Fibroin-Like Protein Fragments

The recombinant silk protein in this disclosure comprises synthetic proteins which are based on repeat units of natural silk proteins. Besides the synthetic repetitive silk protein sequences, these can additionally comprise one or more natural nonrepetitive silk protein sequences. As used herein, “silk fibroin-like protein fragments” refer to protein fragments having a molecular weight and polydispersity as defined herein, and a certain degree of homology to a protein selected from native silk protein, fibroin heavy chain, fibroin light chain, or any protein comprising one or more GAGAGS (SEQ ID NO:2) hexa amino acid repeating units. In some embodiments, a degree of homology is selected from about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, or less than 75%.

As described herein, a protein such as native silk protein, fibroin heavy chain, fibroin light chain, or any protein comprising one or more GAGAGS (SEQ ID NO:2) hexa amino acid repeating units includes between about 9% and about 45% glycine, or about 9% glycine, or about 10% glycine, about 43% glycine, about 44% glycine, about 45% glycine, or about 46% glycine. As described herein, a protein such as native silk protein, fibroin heavy chain, fibroin light chain, or any protein comprising one or more GAGAGS (SEQ ID NO:2) hexa amino acid repeating units includes between about 13% and about 30% alanine, or about 13% alanine, or about 28% alanine, or about 29% alanine, or about 30% alanine, or about 31% alanine. As described herein, a protein such as native silk protein, fibroin heavy chain, fibroin light chain, or any protein comprising one or more GAGAGS (SEQ ID NO:2) hexa amino acid repeating units includes between 9% and about 12% serine, or about 9% serine, or about 10% serine, or about 11% serine, or about 12% serine.

In some embodiments, a silk fibroin-like protein described herein includes about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, or about 55% glycine. In some embodiments, a silk fibroin-like protein described herein includes about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, or about 39% alanine. In some embodiments, a silk fibroin-like protein described herein includes about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, or about 22% serine. In some embodiments, a silk fibroin-like protein described herein may include independently any amino acid known to be included in natural fibroin. In some embodiments, a silk fibroin-like protein described herein may exclude independently any amino acid known to be included in natural fibroin. In some embodiments, on average 2 out of 6 amino acids, 3 out of 6 amino acids, or 4 out of 6 amino acids in a silk fibroin-like protein described herein is glycine. In some embodiments, on average 1 out of 6 amino acids, 2 out of 6 amino acids, or 3 out of 6 amino acids in a silk fibroin-like protein described herein is alanine. In some embodiments, on average none out of 6 amino acids, 1 out of 6 amino acids, or 2 out of 6 amino acids in a silk fibroin-like protein described herein is serine.

### Sericin or Sericin Fragments

The main body of the raw silk is silk fibroin fiber, and the silk fibroin fiber is coated with an adhesive substance silk sericin. Sericin is a colloidal silk protein that covers the surface of the silk thread and is composed of bulky amino acids rich in chemical reactivity such as serine, threonine, and aspartic acid, in addition to glycine and alanine. In the various processes of producing silk from raw silk, sericin is important in controlling the solubility of silk and producing high quality silk. Moreover, it plays an extremely important role as an adhesion functional protein. When silk fiber is used as a clothing material, most of the silk sericin covering the silk thread is removed and discarded, so sericin is a valuable unused resource.

In some embodiments, the silk protein fragments described herein include sericin or sericin fragments. Methods of preparing sericin or sericin fragments and their applications in various fields are known and are described herein, and are also described, for example, in U.S. Pat. Nos. 7,115,388, 7,157,273, and 9,187,538, all of which are incorporated by reference herein in their entireties.

In some embodiments, sericin removed from the raw silk cocoons, such as in a degumming step, can be collected and used in the methods described herein. Sericin can also be reconstituted from a powder, and used within the compositions and methods of the disclosure.

### Other Properties of SPF

Compositions of the present disclosure are “biocompatible” or otherwise exhibit “biocompatibility” meaning that the compositions are compatible with living tissue or a living system by not being toxic, injurious, or physiologically reactive and not causing immunological rejection or an inflammatory response. Such biocompatibility can be evidenced by participants topically applying compositions of the present disclosure on their skin for an extended period of time. In an embodiment, the extended period of time is about 3 days. In an embodiment, the extended period of time is about 7 days. In an embodiment, the extended period of time is about 14 days. In an embodiment, the extended period of time is about 21 days. In an embodiment, the extended period of time is about 30 days. In an embodiment, the extended period of time is selected from the group consisting of about 1 month, about 2 months, about 3 months, about 4 months, about 5 months, about 6 months, about 7 months, about 8 months, about 9 months, about 10 months, about 11 months, about 12 months, and indefinitely. For example, in some embodiments, the coatings described herein are biocompatible coatings.

In some embodiments, compositions described herein, which may be biocompatible compositions (e.g., biocompatible coatings that include silk), may be evaluated and comply with International Standard ISO 10993-1, titled the “Biological evaluation of medical devices—Part 1: Evaluation and testing within a risk management process.” In some embodiments, compositions described herein, which may be biocompatible compositions, may be evaluated under ISO 106993-1 for one or more of cytotoxicity, sensitization, hemocompatibility, pyrogenicity, implantation, genotoxicity, carcinogenicity, reproductive and developmental toxicity, and degradation.

Compositions of the present disclosure are “hypoallergenic” meaning that they are relatively unlikely to cause an allergic reaction. Such hypoallergenicity can be evidenced by participants topically applying compositions of the present disclosure on their skin for an extended period of time. In an embodiment, the extended period of time is about 3 days. In an embodiment, the extended period of time is about 7 days. In an embodiment, the extended period of time is about 14 days. In an embodiment, the extended period of time is about 21 days. In an embodiment, the extended period of time is about 30 days. In an embodiment, the extended period of time is selected from the group consisting of about 1 month, about 2 months, about 3 months, about 4 months, about 5 months, about 6 months, about 7 months, about 8 months, about 9 months, about 10 months, about 11 months, about 12 months, and indefinitely.

In an embodiment, the stability of a composition of the present disclosure is about 1 day. In an embodiment, the stability of a composition of the present disclosure is about 2 days. In an embodiment, the stability of a composition of the present disclosure is about 3 days. In an embodiment, the stability of a composition of the present disclosure is about 4 days. In an embodiment, the stability of a composition of the present disclosure is about 5 days. In an embodiment, the stability of a composition of the present disclosure is about 6 days. In an embodiment, the stability of a composition of the present disclosure is about 7 days. In an embodiment, the stability of a composition of the present disclosure is about 8 days. In an embodiment, the stability of a composition of the present disclosure is about 9 days. In an embodiment, the stability of a composition of the present disclosure is about 10 days.

In an embodiment, the stability of a composition of the present disclosure is about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, or about 30 days.

In an embodiment, the stability of a composition of the present disclosure is 10 days to 6 months. In an embodiment, the stability of a composition of the present disclosure is 6 months to 12 months. In an embodiment, the stability of a composition of the present disclosure is 12 months to 18 months. In an embodiment, the stability of a composition of the present disclosure is 18 months to 24 months. In an embodiment, the stability of a composition of the present disclosure is 24 months to 30 months. In an embodiment, the stability of a composition of the present disclosure is 30 months to 36 months. In an embodiment, the stability of a composition of the present disclosure is 36 months to 48 months. In an embodiment, the stability of a composition of the present disclosure is 48 months to 60 months.

In an embodiment, a SPF composition of the present disclosure is not soluble in an aqueous solution due to the crystallinity of the protein. In an embodiment, a SPF composition of the present disclosure is soluble in an aqueous solution. In an embodiment, the SPF of a composition of the present disclosure include a crystalline portion of about two-thirds and an amorphous region of about one-third. In an embodiment, the SPF of a composition of the present disclosure include a crystalline portion of about one-half and an amorphous region of about one-half. In an embodiment, the SPF of a composition of the present disclosure include a 99% crystalline portion and a 1% amorphous region. In an embodiment, the SPF of a composition of the present disclosure include a 95% crystalline portion and a 5% amorphous region. In an embodiment, the SPF of a composition of the present disclosure include a 90% crystalline portion and a 10% amorphous region. In an embodiment, the SPF of a composition of the present disclosure include a 85% crystalline portion and a 15% amorphous region. In an embodiment, the SPF of a composition of the present disclosure include a 80% crystalline portion and a 20% amorphous region. In an embodiment, the SPF of a composition of the present disclosure include a 75% crystalline portion and a 25% amorphous region. In an embodiment, the SPF of a composition of the present disclosure include a 70% crystalline portion and a 30% amorphous region. In an embodiment, the SPF of a composition of the present disclosure include a 65% crystalline portion and a 35% amorphous region. In an embodiment, the SPF of a composition of the present disclosure include a 60% crystalline portion and a 40% amorphous region. In an embodiment, the SPF of a composition of the present disclosure include a 50% crystalline portion and a 50% amorphous region. In an embodiment, the SPF of a composition of the present disclosure include a 40% crystalline portion and a 60% amorphous region. In an embodiment, the SPF of a composition of the present disclosure include a 35% crystalline portion and a 65% amorphous region. In an embodiment, the SPF of a composition of the present disclosure include a 30% crystalline portion and a 70% amorphous region. In an embodiment, the SPF of a composition of the present disclosure include a 25% crystalline portion and a 75% amorphous region. In an embodiment, the SPF of a composition of the present disclosure include a 20% crystalline portion and a 80% amorphous region. In an embodiment, the SPF of a composition of the present disclosure include a 15% crystalline portion and a 85% amorphous region. In an embodiment, the SPF of a composition of the present disclosure include a 10% crystalline portion and a 90% amorphous region. In an embodiment, the SPF of a composition of the present disclosure include a 5% crystalline portion and a 90% amorphous region. In an embodiment, the SPF of a composition of the present disclosure include a 1% crystalline portion and a 99% amorphous region.

As used herein, the term “substantially free of inorganic residuals” means that the composition exhibits residuals of 0.1% (w/w) or less. In an embodiment, substantially free of inorganic residuals refers to a composition that exhibits residuals of 0.05% (w/w) or less. In an embodiment, substantially free of inorganic residuals refers to a composition that exhibits residuals of 0.01% (w/w) or less. In an embodiment, the amount of inorganic residuals is between 0 ppm (“non-detectable” or “ND”) and 1000 ppm. In an embodiment, the amount of inorganic residuals is ND to about 500 ppm. In an embodiment, the amount of inorganic residuals is ND to about 400 ppm. In an embodiment, the amount of inorganic residuals is ND to about 300 ppm. In an embodiment, the amount of inorganic residuals is ND to about 200 ppm. In an embodiment, the amount of inorganic residuals is ND to about 100 ppm. In an embodiment, the amount of inorganic residuals is between 10 ppm and 1000 ppm.

As used herein, the term “substantially free of organic residuals” means that the composition exhibits residuals of 0.1% (w/w) or less, in an embodiment, substantially free of organic residuals refers to a composition that exhibits residuals of 0.05% (w/w) or less. In an embodiment, substantially free of organic residuals refers to a composition that exhibits residuals of 0.01% (w/w) or less. In an embodiment, the amount of organic residuals is between 0 ppm (“non-detectable” or “ND”) and 1000 ppm. In an embodiment, the amount of organic residuals is ND to about 500 ppm. In an embodiment, the amount of organic residuals is ND to about 400 ppm. In an embodiment, the amount of organic residuals is ND to about 300 ppm. In an embodiment, the amount of organic residuals is ND to about 200 ppm. In an embodiment, the amount of organic residuals is ND to about 100 ppm. In an embodiment, the amount of organic residuals is between 10 ppm and 1000 ppm.

Compositions of the present disclosure exhibit “biocompatibility” meaning that the compositions are compatible with living tissue or a living system by not being toxic, injurious, or physiologically reactive and not causing immunological rejection. Such biocompatibility can be evidenced by participants topically applying compositions of the present disclosure on their skin for an extended period of time. In an embodiment, the extended period of time is about 3 days. In an embodiment, the extended period of time is about 7 days, in an embodiment, the extended period of time is about 14 days, in an embodiment, the extended period of time is about 21 days. In an embodiment, the extended period of time is about 30 days. In an embodiment, the extended period of time is selected from the group consisting of about I month, about 2 months, about 3 months, about 4 months, about 5 months, about 6 months, about 7 months, about 8 months, about 9 months, about 10 months, about 11 months, about 12 months, and indefinitely.

Compositions of the present disclosure are “hypoallergenic” meaning that they are relatively unlikely to cause an allergic reaction. Such hypoallergenicity can be evidenced by participants topically applying compositions of the present disclosure on their skin for an extended period of time. In an embodiment, the extended period of time is about 3 days. In an embodiment, the extended period of time is about 7 days. In an embodiment, the extended period of time is about 14 days. In an embodiment, the extended period of time is about 21 days. In an embodiment, the extended period of time is about 30 days. In an embodiment, the extended period of time is selected from the group consisting of about 1 month, about 2 months, about 3 months, about 4 months, about 5 months, about 6 months, about 7 months, about 8 months, about 9 months, about 10 months, about 11 months, about 12 months, and indefinitely.

Following are non-limiting examples of suitable ranges for various parameters in and for preparation of the silk solutions of the present disclosure. The silk solutions of the present disclosure may include one or more, but not necessarily all, of these parameters and may be prepared using various combinations of ranges of such parameters.

In an embodiment, the percent SPF in the solution is less than 30.0 wt. %. In an embodiment, the percent SPF in the solution is less than 25.0 wt. %. In an embodiment, the percent SPF in the solution is less than 20.0 wt. %. In an embodiment, the percent SPF in the solution is less than 19.0 wt. %. In an embodiment, the percent SPF in the solution is less than 18.0 wt. %. In an embodiment, the percent SPF in the solution is less than 17.0 wt. %. In an embodiment, the percent SPF in the solution is less than 16.0 wt. %. In an embodiment, the percent SPF in the solution is less than 15.0 wt. %. In an embodiment, the percent SPF in the solution is less than 14.0 wt. %. In an embodiment, the percent SPF in the solution is less than 13.0 wt. %. In an embodiment, the percent SPF in the solution is less than 12.0 wt. %. In an embodiment, the percent SPF in the solution is less than 11.0 wt. %. In an embodiment, the percent SPF in the solution is less than 10.0 wt. %. In an embodiment, the percent SPF in the solution is less than 9.0 wt. %. In an embodiment, the percent SPF in the solution is less than 8.0 wt. %. In an embodiment, the percent SPF in the solution is less than 7.0 wt. %. In an embodiment, the percent SPF in the solution is less than 6.0 wt. %. In an embodiment, the percent SPF in the solution is less than 5.0 wt. %. In an embodiment, the percent SPF in the solution is less than 4.0 wt. %. In an embodiment, the percent SPF in the solution is less than 3.0 wt. %. In an embodiment, the percent SPF in the solution is less than 2.0 wt. %. In an embodiment, the percent SPF in the solution is less than 1.0 wt. %. In an embodiment, the percent SPF in the solution is less than 0.9 wt. %. In an embodiment, the percent SPF in the solution is less than 0.8 wt. %. In an embodiment, the percent SPF in the solution is less than 0.7 wt. %. In an embodiment, the percent SPF in the solution is less than 0.6 wt. %. In an embodiment, the percent SPF in the solution is less than 0.5 wt. %. In an embodiment, the percent SPF in the solution is less than 0.4 wt. %. In an embodiment, the percent SPF in the solution is less than 0.3 wt. %. In an embodiment, the percent SPF in the solution is less than 0.2 wt. %. In an embodiment, the percent SPF in the solution is less than 0.1 wt. %.

In an embodiment, the percent SPF in the solution is greater than 0.1 wt. %. In an embodiment, the percent SPF in the solution is greater than 0.2 wt. %. In an embodiment, the percent SPF in the solution is greater than 0.3 wt. %. In an embodiment, the percent SPF in the solution is greater than 0.4 wt. %. In an embodiment, the percent SPF in the solution is greater than 0.5 wt. %. In an embodiment, the percent SPF in the solution is greater than 0.6 wt. %. In an embodiment, the percent SPF in the solution is greater than 0.7 wt. %. In an embodiment, the percent SPF in the solution is greater than 0.8 wt. %. In an embodiment, the percent SPF in the solution is greater than 0.9 wt. %. In an embodiment, the percent SPF in the solution is greater than 1.0 wt. %. In an embodiment, the percent SPF in the solution is greater than 2.0 wt. %. In an embodiment, the percent SPF in the solution is greater than 3.0 wt. %. In an embodiment, the percent SPF in the solution is greater than 4.0 wt. %. In an embodiment, the percent SPF in the solution is greater than 5.0 wt. %. In an embodiment, the percent SPF in the solution is greater than 6.0 wt. %. In an embodiment, the percent SPF in the solution is greater than 7.0 wt. %. In an embodiment, the percent SPF in the solution is greater than 8.0 wt. %. In an embodiment, the percent SPF in the solution is greater than 9.0 wt. %. In an embodiment, the percent SPF in the solution is greater than 10.0 wt. %. In an embodiment, the percent SPF in the solution is greater than 11.0 wt. %. In an embodiment, the percent SPF in the solution is greater than 12.0 wt. %. In an embodiment, the percent SPF in the solution is greater than 13.0 wt. %. In an embodiment, the percent SPF in the solution is greater than 14.0 wt. %. In an embodiment, the percent SPF in the solution is greater than 15.0 wt. %. In an embodiment, the percent SPF in the solution is greater than 16.0 wt. %. In an embodiment, the percent SPF in the solution is greater than 17.0 wt. %. In an embodiment, the percent SPF in the solution is greater than 18.0 wt. %. In an embodiment, the percent SPF in the solution is greater than 19.0 wt. %. In an embodiment, the percent SPF in the solution is greater than 20.0 wt. %. In an embodiment, the percent SPF in the solution is greater than 25.0 wt. %.

In an embodiment, the percent SPF in the solution ranges from about 0.1 wt. % to about 30.0 wt. %. In an embodiment, the percent SPF in the solution ranges from about 0.1 wt. % to about 25.0 wt. %. In an embodiment, the percent SPF in the solution ranges from about 0.1 wt. % to about 20.0 wt. %. In an embodiment, the percent SPF in the solution ranges from about 0.1 wt. % to about 15.0 wt. %. In an embodiment, the percent SPF in the solution ranges from about 0.1 wt. % to about 10.0 wt. %. In an embodiment, the percent SPF in the solution ranges from about 0.1 wt. % to about 9.0 wt. %. In an embodiment, the percent SPF in the solution ranges from about 0.1 wt. % to about 8.0 wt. %. In an embodiment, the percent SPF in the solution ranges from about 0.1 wt. % to about 7.0 wt. %. In an embodiment, the percent SPF in the solution ranges from about 0.1 wt. % to about 6.5 wt. %. In an embodiment, the percent SPF in the solution ranges from about 0.1 wt. % to about 6.0 wt. %. In an embodiment, the percent SPF in the solution ranges from about 0.1 wt. % to about 5.5 wt. %. In an embodiment, the percent SPF in the solution ranges from about 0.1 wt. % to about 5.0 wt. %. In an embodiment, the percent SPF in the solution ranges from about 0.1 wt. % to about 4.5 wt. %. In an embodiment, the percent SPF in the solution ranges from about 0.1 wt. % to about 4.0 wt. %. In an embodiment, the percent SPF in the solution ranges from about 0.1 wt. % to about 3.5 wt. %. In an embodiment, the percent SPF in the solution ranges from about 0.1 wt. % to about 3.0 wt. %. In an embodiment, the percent SPF in the solution ranges from about 0.1 wt. % to about 2.5 wt. %. In an embodiment, the percent SPF in the solution ranges from about 0.1 wt. % to about 2.0 wt. %. In an embodiment, the percent SPF in the solution ranges from about 0.1 wt. % to about 2.4 wt. %. In an embodiment, the percent SPF in the solution ranges from about 0.5 wt. % to about 5.0 wt. %. In an embodiment, the percent SPF in the solution ranges from about 0.5 wt. % to about 4.5 wt. %. In an embodiment, the percent SPF in the solution ranges from about 0.5 wt. % to about 4.0 wt. %. In an embodiment, the percent SPF in the solution ranges from about 0.5 wt. % to about 3.5 wt. %. In an embodiment, the percent SPF in the solution ranges from about 0.5 wt. % to about 3.0 wt. %. In an embodiment, the percent SPF in the solution ranges from about 0.5 wt. % to about 2.5 wt. %. In an embodiment, the percent SPF in the solution ranges from about 1.0 wt. % to about 4.0 wt. %. In an embodiment, the percent SPF in the solution ranges from about 1.0 wt. % to about 3.5 wt. %. In an embodiment, the percent SPF in the solution ranges from about 1.0 wt. % to about 3.0 wt. %. In an embodiment, the percent SPF in the solution ranges from about 1.0 wt. % to about 2.5 wt. %. In an embodiment, the percent SPF in the solution ranges from about 1.0 wt. % to about 2.4 wt. %. In an embodiment, the percent SPF in the solution ranges from about 1.0 wt. % to about 2.0 wt. %.

In an embodiment, the percent SPF in the solution ranges from about 20.0 wt. % to about 30.0 wt. %. In an embodiment, the percent SPF in the solution ranges from about 0.1 wt. % to about 10.0 wt. %. In an embodiment, the percent SPF in the solution ranges from about 1.0 wt. % to about 10.0 wt. %. In an embodiment, the percent SPF in the solution ranges from about 2 wt. % to about 10.0 wt. %. In an embodiment, the percent SPF in the solution ranges from about 0.1 wt. % to about 6.0 wt. %. In an embodiment, the percent SPF in the solution ranges from about 6.0 wt. % to about 10.0 wt. %. In an embodiment, the percent SPF in the solution ranges from about 6.0 wt. % to about 8.0 wt. %. In an embodiment, the percent SPF in the solution ranges from about 6.0 wt. % to about 9.0 wt. %. In an embodiment, the percent SPF in the solution ranges from about 10.0 wt. % to about 20.0 wt. %. In an embodiment, the percent SPF in the solution ranges from about 11.0 wt. % to about 19.0 wt. %. In an embodiment, the percent SPF in the solution ranges from about 12.0 wt. % to about 18.0 wt. %. In an embodiment, the percent SPF in the solution ranges from about 13.0 wt. % to about 17.0 wt. %. In an embodiment, the percent SPF in the solution ranges from about 14.0 wt. % to about 16.0 wt. %. In an embodiment, the percent SPF in the solution is about 1.0 wt. %. In an embodiment, the percent SPF in the solution is about 0.5 wt. %. In an embodiment, the percent SPF in the solution is about 1.5 wt. %. In an embodiment, the percent SPF in the solution is about 2.0 wt. %. In an embodiment, the percent SPF in the solution is about 2.4 wt. %. In an embodiment, the percent SPF in the solution is 3.0 wt. %. In an embodiment, the percent SPF in the solution is 3.5 wt. %. In an embodiment, the percent SPF in the solution is about 4.0 wt. %. In an embodiment, the percent SPF in the solution is about 4.5 wt. %. In an embodiment, the percent SPF in the solution is about 5.0 wt. %. In an embodiment, the percent SPF in the solution is about 5.5 wt. %. In an embodiment the percent SPF in the solution is about 6.0 wt. %. In an embodiment, the percent SPF in the solution is about 6.5 wt. %. In an embodiment, the percent SPF in the solution is about 7.0 wt. %. In an embodiment, the percent SPF in the solution is about 7.5 wt. %. In an embodiment, the percent SPF in the solution is about 8.0 wt. %. In an embodiment, the percent SPF in the solution is about 8.5 wt. %. In an embodiment, the percent SPF in the solution is about 9.0 wt. %. In an embodiment, the percent SPF in the solution is about 9.5 wt. %. In an embodiment, the percent SPF in the solution is about 10.0 wt. %.

In an embodiment, the percent sericin in the solution is non-detectable to 25.0 wt. %. In an embodiment, the percent sericin in the solution is non-detectable to 5.0 wt. %. In an embodiment, the percent sericin in the solution is 1.0 wt. %. In an embodiment, the percent sericin in the solution is 2.0 wt. %. In an embodiment, the percent sericin in the solution is 3.0 wt. %. In an embodiment, the percent sericin in the solution is 4.0 wt. %. In an embodiment, the percent sericin in the solution is 5.0 wt. %. In an embodiment, the percent sericin in the solution is 10.0 wt. %. In an embodiment, the percent sericin in the solution is 25.0 wt. %.

In some embodiments, the silk fibroin protein fragments of the present disclosure are shelf stable (they will not slowly or spontaneously gel when stored in an aqueous solution and there is no aggregation of fragments and therefore no increase in molecular weight over time), from 10 days to 3 years depending on storage conditions, percent SPF, and number of shipments and shipment conditions.

Additionally, pH may be altered to extend shelf life and/or support shipping conditions by preventing premature folding and aggregation of the silk. In an embodiment, the stability of the LiBr-silk fragment solution is 0 to 1 year. In an embodiment, the stability of the LiBr-silk fragment solution is 0 to 2 years. In an embodiment, the stability of the LiBr-silk fragment solution is 0 to 3 years. In an embodiment, the stability of the LiBr-silk fragment solution is 0 to 4 years. In an embodiment, the stability of the LiBr-silk fragment solution is 0 to 5 years. In an embodiment, the stability of the LiBr-silk fragment solution is 1 to 2 years. In an embodiment, the stability of the LiBr-silk fragment solution is 1 to 3 years. In an embodiment, the stability of the LiBr-silk fragment solution is 1 to 4 years. In an embodiment, the stability of the LiBr-silk fragment solution is 1 to 5 years. In an embodiment, the stability of the LiBr-silk fragment solution is 2 to 3 years. In an embodiment, the stability of the LiBr-silk fragment solution is 2 to 4 years. In an embodiment, the stability of the LiBr-silk fragment solution is 2 to 5 years. In an embodiment, the stability of the LiBr-silk fragment solution is 3 to 4 years. In an embodiment, the stability of the LiBr-silk fragment solution is 3 to 5 years. In an embodiment, the stability of the LiBr-silk fragment solution is 4 to 5 years.

In an embodiment, the stability of a composition of the present disclosure is 10 days to 6 months. In an embodiment, the stability of a composition of the present disclosure is 6 months to 12 months. In an embodiment, the stability of a composition of the present disclosure is 12 months to 18 months. In an embodiment, the stability of a composition of the present disclosure is 18 months to 24 months. In an embodiment, the stability of a composition of the present disclosure is 24 months to 30 months. In an embodiment, the stability of a composition of the present disclosure is 30 months to 36 months. In an embodiment, the stability of a composition of the present disclosure is 36 months to 48 months. In an embodiment, the stability of a composition of the present disclosure is 48 months to 60 months.

In an embodiment, a composition of the present disclosure having SPF has non-detectable levels of LiBr residuals. In an embodiment, the amount of the LiBr residuals in a composition of the present disclosure is between 10 ppm and 1000 ppm. In an embodiment, the amount of the LiBr residuals in a composition of the present disclosure is between 10 ppm and 300 ppm. In an embodiment, the amount of the LiBr residuals in a composition of the present disclosure is less than 25 ppm. In an embodiment, the amount of the Li Br residuals in a composition of the present disclosure is less than 50 ppm. In an embodiment, the amount of the LiBr residuals in a composition of the present disclosure is less than 75 ppm. In an embodiment, the amount of the LiBr residuals in a composition of the present disclosure is less than 100 ppm. In an embodiment, the amount of the LiBr residuals in a composition of the present disclosure is less than 200 ppm. In an embodiment, the amount of the LiBr residuals in a composition of the present disclosure is less than 300 ppm. In an embodiment, the amount of the LiBr residuals in a composition of the present disclosure is less than 400 ppm. In an embodiment, the amount of the LiBr residuals in a composition of the present disclosure is less than 500 ppm. In an embodiment, the amount of the LiBr residuals in a composition of the present disclosure is less than 600 ppm. In an embodiment, the amount of the LiBr residuals in a composition of the present disclosure is less than 700 ppm. In an embodiment, the amount of the LiBr residuals in a composition of the present disclosure is less than 800 ppm. In an embodiment, the amount of the LiBr residuals in a composition of the present disclosure is less than 900 ppm. In an embodiment, the amount of the LiBr residuals in a composition of the present disclosure is less than 1000 ppm. In an embodiment, the amount of the LiBr residuals in a composition of the present disclosure is non-detectable to 500 ppm. In an embodiment, the amount of the LiBr residuals in a composition of the present disclosure is non-detectable to 450 ppm. In an embodiment, the amount of the LiBr residue in a composition of the present disclosure is non-detectable to 400 ppm. In an embodiment, the amount of the LiBr residuals in a composition of the present disclosure is non-detectable to 350 ppm. In an embodiment, the amount of the LiBr residuals in a composition of the present disclosure is non-detectable to 300 ppm. In an embodiment, the amount of the LiBr residuals in a composition of the present disclosure is non-detectable to 250 ppm. In an embodiment, the amount of the LiBr residuals in a composition of the present disclosure is non-detectable to 200 ppm. In an embodiment, the amount of the LiBr residuals in a composition of the present disclosure is non-detectable to 150 ppm. In an embodiment, the amount of the LiBr residuals in a composition of the present disclosure is non-detectable to 100 ppm. In an embodiment, the amount of the LiBr residuals in a composition of the present disclosure is 100 ppm to 200 ppm. In an embodiment, the amount of the LiBr residuals in a composition of the present disclosure is 200 ppm to 300 ppm. In an embodiment, the amount of the LiBr residuals in a composition of the present disclosure is 300 ppm to 400 ppm. In an embodiment, the amount of the LiBr residuals in a composition of the present disclosure is 400 ppm to 500 ppm.

In an embodiment, a composition of the present disclosure having SPF, has non-detectable levels of Na₂CO₃ residuals. In an embodiment, the amount of the Na₂CO₃ residuals in a composition of the present disclosure is less than 100 ppm. In an embodiment, the amount of the Na₂CO₃ residuals in a composition of the present disclosure is less than 200 ppm. In an embodiment, the amount of the Na₂CO₃ residuals in a composition of the present disclosure is less than 300 ppm. In an embodiment, the amount of the Na₂CO₃ residuals in a composition of the present disclosure is less than 400 ppm. In an embodiment, the amount of the Na₂CO₃ residuals in a composition of the present disclosure is less than 500 ppm. In an embodiment, the amount of the Na₂CO₃ residuals in a composition of the present disclosure is less than 600 ppm. In an embodiment, the amount of the Na₂CO₃ residuals in a composition of the present disclosure is less than 700 ppm. In an embodiment, the amount of the Na₂CO₃ residuals in a composition of the present disclosure is less than 800 ppm. In an embodiment, the amount of the Na₂CO₃ residuals in a composition of the present disclosure is less than 900 ppm. In an embodiment, the amount of the Na₂CO₃ residuals in a composition of the present disclosure is less than 1000 ppm. In an embodiment, the amount of the Na₂CO₃ residuals in a composition of the present disclosure is non-detectable to 500 ppm. In an embodiment, the amount of the Na₂CO₃ residuals in a composition of the present disclosure is non-detectable to 450 ppm. In an embodiment, the amount of the Na₂CO₃ residuals in a composition of the present disclosure is non-detectable to 400 ppm. In an embodiment, the amount of the Na₂CO₃ residuals in a composition of the present disclosure is non-detectable to 350 ppm. In an embodiment, the amount of the Na₂CO₃ residuals in a composition of the present disclosure is non-detectable to 300 ppm. In an embodiment, the amount of the Na₂CO₃ residuals in a composition of the present disclosure is non-detectable to 250 ppm. In an embodiment, the amount of the Na₂CO₃ residuals in a composition of the present disclosure is non-detectable to 200 ppm. In an embodiment, the amount of the Na₂CO₃ residuals in a composition of the present disclosure is non-detectable to 150 ppm. In an embodiment, the amount of the Na₂CO₃ residuals in a composition of the present disclosure is non-detectable to 100 ppm. In an embodiment, the amount of the Na₂CO₃ residuals in a composition of the present disclosure is 100 ppm to 200 ppm. In an embodiment, the amount of the Na₂CO₃ residuals in a composition of the present disclosure is 200 ppm to 300 ppm. In an embodiment, the amount of the Na₂CO₃ residuals in a composition of the present disclosure is 300 ppm to 400 ppm. In an embodiment, the amount of the Na₂CO₃ residuals in a composition of the present disclosure is 400 ppm to 500 ppm.

A unique feature of the SPF compositions of the present disclosure are shelf stability (they will not slowly or spontaneously gel when stored in an aqueous solution and there is no aggregation of fragments and therefore no increase in molecular weight over time), from 10 days to 3 years depending on storage conditions, percent silk, and number of shipments and shipment conditions. Additionally pH may be altered to extend shelf-life and/or support shipping conditions by preventing premature folding and aggregation of the silk. In an embodiment, a SPF solution composition of the present disclosure has a shelf stability for up to 2 weeks at room temperature (RT). In an embodiment, a SPF solution composition of the present disclosure has a shelf stability for up to 4 weeks at RT. In an embodiment, a SPF solution composition of the present disclosure has a shelf stability for up to 6 weeks at RT. In an embodiment, a SPF solution composition of the present disclosure has a shelf stability for up to 8 weeks at RT. In an embodiment, a SPF solution composition of the present disclosure has a shelf stability for up to 10 weeks at RT. In an embodiment, a SPF solution composition of the present disclosure has a shelf stability for up to 12 weeks at RT. In an embodiment, a SPF solution composition of the present disclosure has a shelf stability ranging from about 4 weeks to about 52 weeks at RT.

Table R below shows shelf stability test results for embodiments of SPF compositions of the present disclosure.

TABLE R
Shelf Stability of SPF Compositions of the Present Disclosure

| % Silk | Temperature | Time to Gelation | |
|---|---|---|---|
| 2 | RT | 4 | weeks |
| 2 | 4° C. | >9 | weeks |
| 4 | RT | 4 | weeks |
| 4 | 4° C. | >9 | weeks |
| 6 | RT | 2 | weeks |
| 6 | 4° C. | >9 | weeks |

In some embodiments, the water solubility of the silk film derived from silk fibroin protein fragments as described herein can be modified by solvent annealing (water annealing or methanol annealing), chemical crosslinking, enzyme crosslinking and heat treatment.

In some embodiments, the process of annealing may involve inducing beta-sheet formation in the silk fibroin protein fragment solutions used as a coating material. Techniques of annealing (e.g., increase crystallinity) or otherwise promoting “molecular packing” of silk fibroin-protein based fragments have been described. In some embodiments, the amorphous silk film is annealed to introduce beta-sheet in the presence of a solvent selected from the group of water or organic solvent. In some embodiments, the amorphous silk film is annealed to introduce beta-sheet in the presence of water (water annealing process). In some embodiments, the amorphous silk fibroin protein fragment film is annealed to introduce beta-sheet in the presence of methanol. In some embodiments, annealing (e.g., the beta sheet formation) is induced by addition of an organic solvent. Suitable organic solvents include, but are not limited to methanol, ethanol, acetone, isopropanol, or combination thereof.

In some embodiments, annealing is carried out by so-called “water-annealing” or “water vapor annealing” in which water vapor is used as an intermediate plasticizing agent or catalyst to promote the packing of beta-sheets. In some embodiments, the process of water annealing may be performed under vacuum. Suitable such methods have been described in Jin H-J et al. (2005), Water-stable Silk Films with Reduced Beta-Sheet Content, Advanced Functional Materials, 15: 1241-1247; Xiao H. et al. (2011), Regulation of Silk Material Structure by Temperature-Controlled Water Vapor Annealing, Biomacromolecules, 12(5): 1686-1696.

The important feature of the water annealing process is to drive the formation of crystalline beta-sheet in the silk fibroin protein fragment peptide chain to allow the silk fibroin self-assembling into a continuous film. In some embodiments, the crystallinity of the silk fibroin protein fragment film is controlled by controlling the temperature of water vapor and duration of the annealing. In some embodiments, the annealing is performed at a temperature ranging from about 65° C. to about 110° C. In some embodiments, the temperature of the water is maintained at about 80° C. In some embodiments, annealing is performed at a temperature selected from the group of about 65° C., about 70° C., about 75° C., about 80° C., about 85° C., about 90° C., about 95° C., about 100° C., about 105° C., and about 110° C.

In some embodiments, the annealing process lasts a period of time selected from the group of about 1 minute to about 40 minutes, about 1 minute to about 50 minutes, about 1 minute to about 60 minutes, about 1 minute to about 70 minutes, about 1 minute to about 80 minutes, about 1 minute to about 90 minutes, about 1 minute to about 100 minutes, about 1 minute to about 110 minutes, about 1 minute to about 120 minutes, about 1 minute to about 130 minutes, about 5 minutes to about 40 minutes, about 5 minutes to about 50 minutes, about 5 minutes to about 60 minutes, about 5 minutes to about 70 minutes, about 5 minutes to about 80 minutes, about 5 minutes to about 90 minutes, about 5 minutes to about 100 minutes, about 5 minutes to about 110 minutes, about 5 minutes to about 120 minutes, about 5 minutes to about 130 minutes, about 10 minutes to about 40 minutes, about 10 minutes to about 50 minutes, about 10 minutes to about 60 minutes, about 10 minutes to about 70 minutes, about 10 minutes to about 80 minutes, about 10 minutes to about 90 minutes, about 10 minutes to about 100 minutes, about 10 minutes to about 110 minutes, about 10 minutes to about 120 minutes, about 10 minutes to about 130 minutes, about 15 minutes to about 40 minutes, about 15 minutes to about 50 minutes, about 15 minutes to about 60 minutes, about 15 minutes to about 70 minutes, about 15 minutes to about 80 minutes, about 15 minutes to about 90 minutes, about 15 minutes to about 100 minutes, about 15 minutes to about 110 minutes, about 15 minutes to about 120 minutes, about 15 minutes to about 130 minutes, about 20 minutes to about 40 minutes, about 20 minutes to about 50 minutes, about 20 minutes to about 60 minutes, about 20 minutes to about 70 minutes, about 20 minutes to about 80 minutes, about 20 minutes to about 90 minutes, about 20 minutes to about 100 minutes, about 20 minutes to about 110 minutes, about 20 minutes to about 120 minutes, about 20 minutes to about 130 minutes, about 25 minutes to about 40 minutes, about 25 minutes to about 50 minutes, about 25 minutes to about 60 minutes, about 25 minutes to about 70 minutes, about 25 minutes to about 80 minutes, about 25 minutes to about 90 minutes, about 25 minutes to about 100 minutes, about 25 minutes to about 110 minutes, about 25 minutes to about 120 minutes, about 25 minutes to about 130 minutes, about 30 minutes to about 40 minutes, about 30 minutes to about 50 minutes, about 30 minutes to about 60 minutes, about 30 minutes to about 70 minutes, about 30 minutes to about 80 minutes, about 30 minutes to about 90 minutes, about 30 minutes to about 100 minutes, about 30 minutes to about 110 minutes, about 30 minutes to about 120 minutes, about 30 minutes to about 130 minutes, about 35 minutes to about 40 minutes, about 35 minutes to about 50 minutes, about 35 minutes to about 60 minutes, about 35 minutes to about 70 minutes, about 35 minutes to about 80 minutes, about 35 minutes to about 90 minutes, about 35 minutes to about 100 minutes, about 35 minutes to about 110 minutes, about 35 minutes to about 120 minutes, about 35 minutes to about 130 minutes, about 40 minutes to about 50 minutes, about 40 minutes to about 60 minutes, about 40 minutes to about 70 minutes, about 40 minutes to about 80 minutes, about 40 minutes to about 90 minutes, about 40 minutes to about 100 minutes, about 40 minutes to about 110 minutes, about 40 minutes to about 120 minutes, about 40 minutes to about 130 minutes, about 45 minutes to about 50 minutes, about 45 minutes to about 60 minutes, about 45 minutes to about 70 minutes, about 45 minutes to about 80 minutes, about 45 minutes to about 90 minutes, about 45 minutes to about 100 minutes, about 45 minutes to about 110 minutes, about 45 minutes to about 120 minutes, and about 45 minutes to about 130 minutes. In some embodiments, the annealing process lasts a period of time ranging from about 1 minute to about 60 minutes. In some embodiments, the annealing process lasts a period of time ranging from about 45 minutes to about 60 minutes. The longer water annealing post-processing corresponded an increased crystallinity of silk fibroin protein fragments.

In some embodiments, the annealed silk fibroin protein fragment film is immersing the wet silk fibroin protein fragment film in 100% methanol for 60 minutes at room temperature. The methanol annealing changed the composition of silk fibroin protein fragment film from predominantly amorphous random coil to crystalline antiparallel beta-sheet structure.

In some embodiments, the SPF as described herein can be used to prepare SPF microparticles by precipitation with methanol. Alternative flash drying, fluid-bed drying, spray drying or vacuum drying can be applied to remove water from the silk solution. The SPF powder can then be stored and handled without refrigeration or other special handling procedures. In some embodiments, the SPF powders comprise low molecular weight silk fibroin protein fragments. In some embodiments, the SPF powders comprise mid-molecular weight silk fibroin protein fragments. In some embodiments, the SPF powders comprise a mixture of low molecular weight silk fibroin protein fragments and mid-molecular weight silk fibroin protein fragment.

As used herein, the terms “substantially sericin free” or “substantially devoid of sericin” refer to silk fibers in which a majority of the sericin protein has been removed. In an embodiment, silk fibroin that is substantially devoid of sericin refers to silk fibroin having from about 0.01 wt. % to about 10.0 wt. % sericin. In an embodiment, silk fibroin that is substantially devoid of sericin refers to silk fibroin having about 0.01 wt. % to about 9.0 wt. % sericin. In an embodiment, silk fibroin that is substantially devoid of sericin refers to silk fibroin having from about 0.01 wt. % to about 8.0 wt. % sericin. In an embodiment, silk fibroin that is substantially devoid of sericin refers to silk fibroin having from about 0.01 wt. % to about 7.0 wt. % sericin. In an embodiment, silk fibroin that is substantially devoid of sericin refers to silk fibroin having from about 0.01 wt. % to about 6.0 wt. % sericin. In an embodiment, silk fibroin that is substantially devoid of sericin refers to silk fibroin having from about 0.01 wt. % to about 5.0 wt. % sericin. In an embodiment, silk fibroin that is substantially devoid of sericin refers to silk fibroin having from about 0 wt. % to about 4.0 wt. % sericin. In an embodiment, silk fibroin that is substantially devoid of sericin refers to silk fibroin having from about 0.05 wt. % to about 4.0 wt. % sericin. In an embodiment, silk fibroin that is substantially devoid of sericin refers to silk fibroin having from about 0.1 wt. % to about 4.0 wt. % sericin. In an embodiment, silk fibroin that is substantially devoid of sericin refers to silk fibroin having from about 0.5 wt. % to about 4.0 wt. % sericin. In an embodiment, silk fibroin that is substantially devoid of sericin refers to silk fibroin having from about 1.0 wt. % to about 4.0 wt. % sericin. In an embodiment, silk fibroin that is substantially devoid of sericin refers to silk fibroin having from about 1.5 wt. % to about 4.0 wt. % sericin. In an embodiment, silk fibroin that is substantially devoid of sericin refers to silk fibroin having from about 2.0 wt. % to about 4.0 wt. % sericin. In an embodiment, silk fibroin that is substantially devoid of sericin refers to silk fibroin having from about 2.5 wt. % to about 4.0 wt. % sericin. In an embodiment, silk fibroin that is substantially devoid of sericin refers to silk fibroin having a sericin content from about 0.01 wt. % to about 0.1 wt. %. In an embodiment, silk fibroin that is substantially devoid of sericin refers to silk fibroin having a sericin content below about 0.1 wt. %. In an embodiment, silk fibroin that is substantially devoid of sericin refers to silk fibroin having a sericin content below about 0.05 wt. %. In an embodiment, when a silk source is added to a boiling (100° C.) aqueous solution of sodium carbonate for a treatment time of between about 30 minutes to about 60 minutes, a degumming loss of about 26.0 wt. % to about 31.0 wt. % is obtained. Following are non-limiting examples of suitable ranges for various parameters in and for preparation of the silk solutions of the present disclosure. The silk solutions of the present disclosure may include one or more, but not necessarily all, of these parameters and may be prepared using various combinations of ranges of such parameters.

In an embodiment, the percent SPF in the solution is less than 30.0 wt. %. In an embodiment, the percent SPF in the solution is less than 25.0 wt. %. In an embodiment, the percent SPF in the solution is less than 20.0 wt. %. In an embodiment, the percent SPF in the solution is less than 19.0 wt. %. In an embodiment, the percent SPF in the solution is less than 18.0 wt. %. In an embodiment, the percent SPF in the solution is less than 17.0 wt. %. In an embodiment, the percent SPF in the solution is less than 16.0 wt. %. In an embodiment, the percent SPF in the solution is less than 15.0 wt. %. In an embodiment, the percent SPF in the solution is less than 14.0 wt. %. In an embodiment, the percent SPF in the solution is less than 13.0 wt. %. In an embodiment, the percent SPF in the solution is less than 12.0 wt. %. In an embodiment, the percent SPF in the solution is less than 11.0 wt. %. In an embodiment, the percent SPF in the solution is less than 10.0 wt. %. In an embodiment, the percent SPF in the solution is less than 9.0 wt. %. In an embodiment, the percent SPF in the solution is less than 8.0 wt. %. In an embodiment, the percent SPF in the solution is less than 7.0 wt. %. In an embodiment, the percent SPF in the solution is less than 6.0 wt. %. In an embodiment, the percent SPF in the solution is less than 5.0 wt. %. In an embodiment, the percent SPF in the solution is less than 4.0 wt. %. In an embodiment, the percent SPF in the solution is less than 3.0 wt. %. In an embodiment, the percent SPF in the solution is less than 2.0 wt. %. In an embodiment, the percent SPF in the solution is less than 1.0 wt. %. In an embodiment, the percent SPF in the solution is less than 0.9 wt. %. In an embodiment, the percent SPF in the solution is less than 0.8 wt. %. In an embodiment, the percent SPF in the solution is less than 0.7 wt. %. In an embodiment, the percent SPF in the solution is less than 0.6 wt. %. In an embodiment, the percent SPF in the solution is less than 0.5 wt. %. In an embodiment, the percent SPF in the solution is less than 0.4 wt. %. In an embodiment, the percent SPF in the solution is less than 0.3 wt. %. In an embodiment, the percent SPF in the solution is less than 0.2 wt. %. In an embodiment, the percent SPF in the solution is less than 0.1 wt. %.

In an embodiment, the percent SPF in the solution is greater than 0.1 wt. %. In an embodiment, the percent SPF in the solution is greater than 0.2 wt. %. In an embodiment, the percent SPF in the solution is greater than 0.3 wt. %. In an embodiment, the percent SPF in the solution is greater than 0.4 wt. %. In an embodiment, the percent SPF in the solution is greater than 0.5 wt. %. In an embodiment, the percent SPF in the solution is greater than 0.6 wt. %. In an embodiment, the percent SPF in the solution is greater than 0.7 wt. %. In an embodiment, the percent SPF in the solution is greater than 0.8 wt. %. In an embodiment, the percent SPF in the solution is greater than 0.9 wt. %. In an embodiment, the percent SPF in the solution is greater than 1.0 wt. %. In an embodiment, the percent SPF in the solution is greater than 2.0 wt. %. In an embodiment, the percent SPF in the solution is greater than 3.0 wt. %. In an embodiment, the percent SPF in the solution is greater than 4.0 wt. %. In an embodiment, the percent SPF in the solution is greater than 5.0 wt. %. In an embodiment, the percent SPF in the solution is greater than 6.0 wt. %. In an embodiment, the percent SPF in the solution is greater than 7.0 wt. %. In an embodiment, the percent SPF in the solution is greater than 8.0 wt. %. In an embodiment, the percent SPF in the solution is greater than 9.0 wt. %. In an embodiment, the percent SPF in the solution is greater than 10.0 wt. %. In an embodiment, the percent SPF in the solution is greater than 11.0 wt. %. In an embodiment, the percent SPF in the solution is greater than 12.0 wt. %. In an embodiment, the percent SPF in the solution is greater than 13.0 wt. %. In an embodiment, the percent SPF in the solution is greater than 14.0 wt. %. In an embodiment, the percent SPF in the solution is greater than 15.0 wt. %. In an embodiment, the percent SPF in the solution is greater than 16.0 wt. %. In an embodiment, the percent SPF in the solution is greater than 17.0 wt. %. In an embodiment, the percent SPF in the solution is greater than 18.0 wt. %. In an embodiment, the percent SPF in the solution is greater than 19.0 wt. %. In an embodiment, the percent SPF in the solution is greater than 20.0 wt. %. In an embodiment, the percent SPF in the solution is greater than 25.0 wt. %. In an embodiment, the percent SPF in the solution ranges from about 0.1 wt. % to about 30.0 wt. %. In an embodiment, the percent SPF in the solution ranges from about 0.1 wt. % to about 25.0 wt. %. In an embodiment, the percent SPF in the solution ranges from about 0.1 wt. % to about 20.0 wt. %. In an embodiment, the percent SPF in the solution ranges from about 0.1 wt. % to about 15.0 wt. %. In an embodiment, the percent SPF in the solution ranges from about 0.1 wt. % to about 10.0 wt. %. In an embodiment, the percent SPF in the solution ranges from about 0.1 wt. % to about 9.0 wt. %. In an embodiment, the percent SPF in the solution ranges from about 0.1 wt. % to about 8.0 wt. %. In an embodiment, the percent SPF in the solution ranges from about 0.1 wt. % to about 7.0 wt. %. In an embodiment, the percent SPF in the solution ranges from about 0.1 wt. % to about 6.5 wt. %. In an embodiment, the percent SPF in the solution ranges from about 0.1 wt. % to about 6.0 wt. %. In an embodiment, the percent SPF in the solution ranges from about 0.1 wt. % to about 5.5 wt. %. In an embodiment, the percent SPF in the solution ranges from about 0.1 wt. % to about 5.0 wt. %. In an embodiment, the percent SPF in the solution ranges from about 0.1 wt. % to about 4.5 wt. %. In an embodiment, the percent SPF in the solution ranges from about 0.1 wt. % to about 4.0 wt. %. In an embodiment, the percent SPF in the solution ranges from about 0.1 wt. % to about 3.5 wt. %. In an embodiment, the percent SPF in the solution ranges from about 0.1 wt. % to about 3.0 wt. %. In an embodiment, the percent SPF in the solution ranges from about 0.1 wt. % to about 2.5 wt. %. In an embodiment, the percent SPF in the solution ranges from about 0.1 wt. % to about 2.0 wt. %. In an embodiment, the percent SPF in the solution ranges from about 0.1 wt. % to about 2.4 wt. %. In an embodiment, the percent SPF in the solution ranges from about 0.5 wt. % to about 5.0 wt. %. In an embodiment, the percent SPF in the solution ranges from about 0.5 wt. % to about 4.5 wt. %. In an embodiment, the percent SPF in the solution ranges from about 0.5 wt. % to about 4.0 wt. %. In an embodiment, the percent SPF in the solution ranges from about 0.5 wt. % to about 3.5 wt. %. In an embodiment, the percent SPF in the solution ranges from about 0.5 wt. % to about 3.0 wt. %. In an embodiment, the percent SPF in the solution ranges from about 0.5 wt. % to about 2.5 wt. %. In an embodiment, the percent SPF in the solution ranges from about 1.0 wt. % to about 4.0 wt. %. In an embodiment, the percent SPF in the solution ranges from about 1.0 wt. % to about 3.5 wt. %. In an embodiment, the percent SPF in the solution ranges from about 1.0 wt. % to about 3.0 wt. %. In an embodiment, the percent SPF in the solution ranges from about 1.0 wt. % to about 2.5 wt. %. In an embodiment, the percent SPF in the solution ranges from about 1.0 wt. % to about 2.4 wt. %. In an embodiment, the percent SPF in the solution ranges from about 1.0 wt. % to about 2.0 wt. %.

In an embodiment, the percent SPF in the solution ranges from about 20.0 wt. % to about 30.0 wt. %. In an embodiment, the percent SPF in the solution ranges from about 0.1 wt. % to about 10.0 wt. %. In an embodiment, the percent SPF in the solution ranges from about 1.0 wt. % to about 10.0 wt. %. In an embodiment, the percent SPF in the solution ranges from about 2 wt. % to about 10.0 wt. %. In an embodiment, the percent SPF in the solution ranges from about 0.1 wt. % to about 6.0 wt. %. In an embodiment, the percent SPF in the solution ranges from about 6.0 wt. % to about 10.0 wt. %. In an embodiment, the percent SPF in the solution ranges from about 6.0 wt. % to about 8.0 wt. %. In an embodiment, the percent SPF in the solution ranges from about 6.0 wt. % to about 9.0 wt. %. In an embodiment, the percent SPF in the solution ranges from about 10.0 wt. % to about 20.0 wt. %. In an embodiment, the percent SPF in the solution ranges from about 11.0 wt. % to about 19.0 wt. %. In an embodiment, the percent SPF in the solution ranges from about 12.0 wt. % to about 18.0 wt. %. In an embodiment, the percent SPF in the solution ranges from about 13.0 wt. % to about 17.0 wt. %. In an embodiment, the percent SPF in the solution ranges from about 14.0 wt. % to about 16.0 wt. %. In an embodiment, the percent SPF in the solution is about 1.0 wt. %. In an embodiment, the percent SPF in the solution is about 1.5 wt. %. In an embodiment, the percent SPF in the solution is about 2.0 wt. %. In an embodiment, the percent SPF in the solution is about 2.4 wt. %. In an embodiment, the percent SPF in the solution is 3.0 wt. %. In an embodiment, the percent SPF in the solution is 3.5 wt. %. In an embodiment, the percent SPF in the solution is about 4.0 wt. %. In an embodiment, the percent SPF in the solution is about 4.5 wt. %. In an embodiment, the percent SPF in the solution is about 5.0 wt. %. In an embodiment, the percent SPF in the solution is about 5.5 wt. %. In an embodiment the percent SPF in the solution is about 6.0 wt. %. In an embodiment, the percent SPF in the solution is about 6.5 wt. %. In an embodiment, the percent SPF in the solution is about 7.0 wt. %. In an embodiment, the percent SPF in the solution is about 7.5 wt. %. In an embodiment, the percent SPF in the solution is about 8.0 wt. %. In an embodiment, the percent SPF in the solution is about 8.5 wt. %. In an embodiment, the percent SPF in the solution is about 9.0 wt. %. In an embodiment, the percent SPF in the solution is about 9.5 wt. %. In an embodiment, the percent SPF in the solution is about 10.0 wt. %.

In an embodiment, the percent sericin in the solution is non-detectable to 25.0 wt. %. In an embodiment, the percent sericin in the solution is non-detectable to 5.0 wt. %. In an embodiment, the percent sericin in the solution is 1.0 wt. %. In an embodiment, the percent sericin in the solution is 2.0 wt. %. In an embodiment, the percent sericin in the solution is 3.0 wt. %. In an embodiment, the percent sericin in the solution is 4.0 wt. %. In an embodiment, the percent sericin in the solution is 5.0 wt. %. In an embodiment, the percent sericin in the solution is 10.0 wt. %. In an embodiment, the percent sericin in the solution is 25.0 wt. %.

In some embodiments, the silk fibroin-based protein fragments of the present disclosure are shelf stable (they will not slowly or spontaneously gel when stored in an aqueous solution and there is no aggregation of fragments and therefore no increase in molecular weight over time), from 10 days to 3 years depending on storage conditions, percent SPF, and number of shipments and shipment conditions. Additionally, pH may be altered to extend shelf life and/or support shipping conditions by preventing premature folding and aggregation of the silk. In an embodiment, the stability of the LiBr-silk fragment solution is 0 to 1 year. In an embodiment, the stability of the LiBr-silk fragment solution is 0 to 2 years. In an embodiment, the stability of the LiBr-silk fragment solution is 0 to 3 years. In an embodiment, the stability of the LiBr-silk fragment solution is 0 to 4 years. In an embodiment, the stability of the LiBr-silk fragment solution is 0 to 5 years. In an embodiment, the stability of the LiBr-silk fragment solution is 1 to 2 years. In an embodiment, the stability of the LiBr-silk fragment solution is 1 to 3 years. In an embodiment, the stability of the LiBr-silk fragment solution is 1 to 4 years. In an embodiment, the stability of the LiBr-silk fragment solution is 1 to 5 years. In an embodiment, the stability of the LiBr-silk fragment solution is 2 to 3 years. In an embodiment, the stability of the LiBr-silk fragment solution is 2 to 4 years. In an embodiment, the stability of the LiBr-silk fragment solution is 2 to 5 years. In an embodiment, the stability of the LiBr-silk fragment solution is 3 to 4 years. In an embodiment, the stability of the LiBr-silk fragment solution is 3 to 5 years. In an embodiment, the stability of the LiBr-silk fragment solution is 4 to 5 years.

In an embodiment, the stability of a composition of the present disclosure is 10 days to 6 months. In an embodiment, the stability of a composition of the present disclosure is 6 months to 12 months. In an embodiment, the stability of a composition of the present disclosure is 12 months to 18 months. In an embodiment, the stability of a composition of the present disclosure is 18 months to 24 months. In an embodiment, the stability of a composition of the present disclosure is 24 months to 30 months. In an embodiment, the stability of a composition of the present disclosure is 30 months to 36 months. In an embodiment, the stability of a composition of the present disclosure is 36 months to 48 months. In an embodiment, the stability of a composition of the present disclosure is 48 months to 60 months.

In an embodiment, a selected property of the SPF coated articles that may be enhanced as compared to non-coated articles may include one or more of dimensional stability to laundering, dimensional stability to dry cleaning, appearance after laundering, appearance after dry cleaning, colorfastness to laundering, colorfastness to dry cleaning, colorfastness to non-chlorine bleach, seam torque/spirality (on knits), colorfastness to crocking, colorfastness to rubbing, colorfastness to water, colorfastness to light, colorfastness to perspiration, colorfastness to chlorinated pool water, colorfastness to sea water, tensile strength, seam slippage, tearing strength, seam breaking strength, abrasion resistance, pilling resistance, stretch recovery, bursting strength, colorfastness to die transfer in storage (labels), colorfastness to ozone, pile retention, bowing and skewing, colorfastness to saliva, snagging resistance, wrinkle resistance (e.g., appearance of apparel, retention of creases in fabrics, smooth appearance of fabrics), water repellency, water resistance, stain repellant (e.g., water repellency, oil repellency, water/alcohol repellency), vertical wicking, water absorption, dry rate, soil release, air permeability, wicking, antimicrobial properties, ultraviolet protection, resistance to torque, malodor resistant, biocompatibility, wetting time, absorption rate, spreading speed, accumulative one-way transport, flame retardant properties, coloring properties, fabric softening properties, a pH adjusting property, an antifelting property, and overall moisture management capability.

In any of the foregoing embodiments, at least one property of the article is improved, wherein the property that is improved is dimensional stability to laundering, and wherein the property is improved by an amount relative to an uncoated article selected from the group consisting of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 200%, at least 300%, at least 400%, and at least 500%.

In any of the foregoing embodiments, at least one property of the article is improved, wherein the property that is improved is size retention on laundering, and wherein the property is improved by an amount relative to an uncoated article selected from the group consisting of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 200%, at least 300%, at least 400%, and at least 500%.

In any of the foregoing embodiments, at least one property of the article is improved, wherein the property that is improved is resistance to shrinkage, and wherein the property is improved by an amount relative to an uncoated article selected from the group consisting of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 200%, at least 300%, at least 400%, and at least 500%.

In any of the foregoing embodiments the fabric can be pretreated with a fixing agent component. In some embodiments, the fixing agent is cationic. In some embodiments, the fixing agent is a polyacrylamide. In some embodiments, the fixing agent component comprises a polyacrylamide, a solvent (e.g., water), and an alcohol (e.g, glycol). In some embodiments, the cationic fixing agent provides improved absorbency. In some embodiments the cationic fixing agent has a concentration of about 1 g/L, about 2 g/L, about 3 g/L, about 4 g/L, about 5 g/L, about 6 g/L, about 7 g/L, about 8 g/L, about 9 g/L, or about 10 g/L.

### Compositions and Processes Including Silk Protein Fragment Coatings

In an embodiment, the disclosure may include textiles, such as fibers, yarns, fabrics, or other materials and combinations thereof, that may be coated with an SPF mixture solution (i.e., silk fibroin solution (SFS)) as described herein to produce a coated article. In an embodiment, the coated articles described herein may be treated with additional chemical agents that may enhance the properties of the coated article. In an embodiment, the SFS may include one or more chemical agents that may enhance the properties of the coated article.

In an embodiment, textiles may be flexible materials (woven or non-woven) that include a network of natural and/or man-made fibers, thread, yarn, or a combination thereof. SFS may be applied at any stage of textile processing from individual fibers, to yarn, to fabric, to thread, or a combination thereof. In an embodiment, fibers may be natural fibers that may include a natural fiber cellulose base, wherein the natural fiber cellulose base may include one or more of: (1) a baste such as flax, hemp, kenaf, jute, linen, and/or ramie; (2) a leaf such as flax, hemp, sisal, abaca, banana, henequen, ramie, sunn, and/or coir; and (3) seed hair such as cotton and/or kapok. In an embodiment, fibers may be natural fibers that may include a natural fiber protein base, wherein the natural fiber protein base may include one or more of: (1) hair such as alpaca, camel, cashmere, llama, mohair, and/or vicuna; (2) wool such as sheep; (3) filament such as silk. In an embodiment, fibers may be natural fibers that may include a natural fiber mineral base, including asbestos. In an embodiment, fibers may be man-made fibers that may include a man-made fiber organic natural polymer base, which may include one or more of: (1) a cellulose base such as bamboo, rayon, lyocell, acetate, and/or triacetate; (2) a protein base such as azlon; (3) an alginate; and (4) rubber. In an embodiment, fibers may be man-made fibers that may include a man-made fiber organic synthetic base, which may include one or more of acrylic, anidex, aramid, fluorocarbon, modacrylic, novoloid, nylon, nytril, olefin, PBI, polycarbonate, polyester, rubber, saran, spandex, vinal vinvon. In an embodiment, fibers may be man-made fibers that may include a man-made fiber inorganic base, which may include one or more of a glass material, metallic material, and carbon material.

In an embodiment, yarn may include natural fibers that may include a natural fiber cellulose base, wherein the natural fiber cellulose base may be from: (1) a baste such as flax, hemp, kenaf, jute, linen, and/or ramie; (2) a leaf such as flax, hemp, sisal, abaca, banana, henequen, ramie, sunn, and/or coir; or (3) seed hair such as cotton and/or kapok. In an embodiment, yarn may include natural fibers that may include a natural fiber protein base, wherein the natural fiber protein base may be from: (1) hair such as alpaca, camel, cashmere, llama, mohair, and/or vicuna; (2) wool such as sheep; or (3) filament such as silk. In an embodiment, yarn may include natural fibers that may include a natural fiber mineral base, including asbestos. In an embodiment, yarn may include man-made fibers that may include a man-made fiber organic natural polymer base, which may include: (1) a cellulose base such as bamboo, rayon, lyocell, acetate, and/or triacetate; (2) a protein base such as azlon; (3) an alginate; or (4) rubber. In an embodiment, yarn may include man-made fibers that may include a man-made fiber organic synthetic base, which may include acrylic, anidex, aramid, fluorocarbon, modacrylic, novoloid, nylon, nytril, olefin, PBI, polycarbonate, polyester, rubber, saran, spandex, vinal and/or vinvon. In an embodiment, yarn may include man-made fibers that may include a man-made fiber inorganic base, which may include a glass material, metallic material, carbon material, and/or specialty material.

In an embodiment, fabrics may include natural fibers and/or yarn that may include a natural fiber cellulose base, wherein the natural fiber cellulose base may be from: (1) a baste such as flax, hemp, kenaf, jute, linen, and/or ramie; (2) a leaf such as flax, hemp, sisal, abaca, banana, henequen, ramie, sunn, and/or coir; or (3) seed hair such as cotton and/or kapok. In an embodiment, fabric may include natural fibers and/or yarn that may include a natural fiber protein base, wherein the natural fiber protein base may be from: (1) hair such as alpaca, camel, cashmere, llama, mohair, and/or vicuna; (2) wool such as sheep; or (3) filament such as silk. In an embodiment, fabric may include natural fibers and/or yarn that may include a natural fiber mineral base, including asbestos. In an embodiment, fabric may include man-made fibers and/or yarn that may include a man-made fiber organic natural polymer base, which may include: (1) a cellulose base such as bamboo, rayon, lyocell, acetate, and/or triacetate; (2) a protein base such as azlon; (3) an alginate; or (4) rubber. In an embodiment, fabric may include man-made fibers and/or yarn that may include a man-made fiber organic synthetic base, which may include acrylic, anidex, aramid, fluorocarbon, modacrylic, novoloid, nylon, nytril, olefin, PBI, polycarbonate, polyester, rubber, saran, spandex, vinal and/or vinvon. In an embodiment, fabric may include man-made fibers and/or yarn that may include a man-made fiber inorganic base, which may include a glass material, metallic material, carbon material, and/or specialty material.

In some embodiments, the fabric may comprise alpaca fiber, alpaca fleece, alpaca wool, lama fiber, lama fleece, lama wool, cotton, sheep fleece, sheep wool, byssus, chiengora, qiviut, yak, rabbit, lambswool, mohair wool, tibetan wool, lopi, camel hair, pashmina, angora wool, silkworm silk, spider silk, abaca fiber, coir fiber, flax fiber, jute fiber, kapok fiber, kenaf fiber, raffia fiber, bamboo fiber, hemp, modal fiber, pina, ramie, sisal, soy protein fiber, polyester, polyamide, polyaramid, polytetrafluoroethylene, polyethylene, polypropylene, polyurethane, silicone, mixtures of polyurethane and polyethyleneglycol, ultrahigh molecular weight polyethylene, high-performance polyethylene, nylon, LYCRA (polyester-polyurethane copolymer, also known as SPANDEX and elastomer), or a mixture thereof. In some embodiments, the fabric comprises wool. In some embodiments, the fabric comprises an inert synthetic material, such as polyester, polyamide, polyaramid, polytetrafluoroethylene, polyethylene, polypropylene, polyurethane, silicone, mixtures of polyurethane and polyethyleneglycol, ultrahigh molecular weight polyethylene, high-performance polyethylene, nylon, LYCRA (polyester-polyurethane copolymer, also known as SPANDEX and elastomer), rayon, or a mixture thereof.

In some embodiments, the fabric comprises one or more selected from the group consisting of cotton, silk, alpaca fleece, alpaca wool, lama fleece, lama wool, cotton, cashmere, sheep fleece, sheep wool, and combinations thereof. In some embodiments, the fabric comprises one or more of natural wool, synthetic wool, alpaca fleece, alpaca wool, lama fleece, lama wool, cashmere, sheep fleece, sheep wool, mohair wool, camel hair, or angora wool.

In some embodiments, an article described herein may include a synthetic fiber component in an amount, by weight of the article (w/w), of 100%. In some embodiments, an article described herein may include a synthetic fiber component in an amount, by weight of the article (w/w), of greater than 1%, 2%, 3%, 4%, 5%, 6%, 7%, 8%, 90%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, where the balance of the article, by weight (w/w), is a non-synthetic fiber component, as described herein. In some embodiments, an article described herein may include a synthetic fiber component in an amount, by weight of the article (w/w), of less than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, where the balance of the article, by weight (w/w), is a non-synthetic fiber component, as described herein. In some embodiments, an article described herein may include a synthetic fiber component in an amount, by weight of the article (w/w), of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, where the balance of the article, by weight (w/w), is a non-synthetic fiber component, as described herein.

In some embodiments, the coating further comprises a crosslinker. In some embodiments, any SPF described herein, including silk fibroin or silk fibroin-based protein fragments are chemically modified with a precursor linker comprising a crosslinker to form silk conjugates. In some embodiments, the fabric is covalently linked to the crosslinker. In some embodiments, the crosslinker is covalently linked to the surfactant and/or emulsifier. In some embodiments, the crosslinker is covalently linked to the fabric and to the surfactant and/or emulsifier. In some embodiments, the crosslinker is covalently linked to the fabric and an SPF.

Precursor linkers for coatings can be selected from any of the following natural crosslinkers: caffeic acid, tannic acid, genipin, proanthocyanidin, and the like. Precursor crosslinking can be selected from any of the following enzymatic crosslinking: transglutaminase transferase crosslinking, hydrolase crosslinking, peptidase crosslinking (e.g., sortase SrtA from Staphylococcus aureus), oxidoreductase crosslinking, tyrosinase crosslinking, laccase crosslinking, peroxidase crosslinking (e.g., horseradish peroxidase), lysyl oxidase crosslinking, peptide ligases (e.g., butelase 1, peptiligase, subtiligase, etc.), and the like.

In some embodiments, silk fibroin or silk fibroin-based protein fragments are chemically modified with a precursor linker to form silk conjugates with a crosslinker or an activator independently selected from a N-hydroxysuccinimide ester crosslinker, an imidoester crosslinker, a sulfosuccinimidyl aminobenzoate, a methacrylate, a silane, a silicate, an alkyne compound, an azide compound, an aldehyde, a carbodiimide crosslinker, a dicyclohexyl carbodiimide activator, a dicyclohexyl carbodiimide crosslinker, a maleimide crosslinker, a haloacetyl crosslinker, a pyridyl disulfide crosslinker, a hydrazide crosslinker, an alkoxyamine crosslinker, a reductive amination crosslinker, an aryl azide crosslinker, a diazirine crosslinker, an azide-phosphine crosslinker, a transferase crosslinker, a hydrolase crosslinker, a transglutaminase crosslinker, a peptidase crosslinker, an oxidoreductase crosslinker, a tyrosinase crosslinker, a laccase crosslinker, a peroxidase crosslinker, a lysyl oxidase crosslinker, and any combinations thereof. Some chemically modified silk fibroin has been described in J Mater Chem. 2009, June 23, 19(36), 6443-6450, including cyanuric chloride-activated coupling, carbodiimide coupling, arginine masking, chlorosulfonic acid reaction, diazonium coupling, tyrosinase-catalyzed grafting, and poly(methacrylate) grafting.

In some embodiments, the article further comprises a crosslinking agent. In some embodiments, the crosslinking agent is a polyphenol compound comprising 12 phenolic hydroxyl groups, having a molecular weight of about 500-4000 Da, and exhibiting about 5-7 aromatic rings per 1000 Da. In some embodiments, the crosslinking agent is a polyphenol compound selected from the group consisting of curcumin, desmethoxycurcumin, bis-desmethoxycurcumin, resveratrol, caffeic acid, tannin, gallotannin, procyanidin, hydrolysable tannin, phlorotannin, gallic acid, chlorogenic acid, carnosol, capsaicin, 6-shogaol, 6-gingerol, flavonoid, flavanol, neoflavonoid, arbutin, cynarin, apigenin, isocuttelarein, luteolin, nobiletin, tangeretin, tectochrysin, galangin, kaempferol, myricetin, quercetin, rutin, citrin, curcurocitrin, eriodictyol, hesperidin, naringenin, naringin, pinocembrin, quercitrin, biochanin A, chrysin, daidzein, equol, formononetin, genistein, glycetein, ipriflavone, lactuin, pycnogenol, silymarin, lignin, and combinations thereof.

In an embodiment, the coating is applied to an article including a fabric at the yarn level. In an embodiment, the coating is applied at the fabric level. In an embodiment, the coating has a thickness selected from the group consisting of about 5 nm, about 10 nm, about 15 nm, about 20 nm, about 25 nm, about 50 nm, about 100 nm, about 200 nm, about 500 nm, about 1 μm, about 5 μm, about 10 μm, and about 20 μm. In an embodiment, the coating has a thickness range selected from the group consisting of about 5 nm to about 100 nm, about 100 nm to about 200 nm, about 200 nm to about 500 nm, about 1 μm to about 2 μm, about 2 μm to about 5 μm, about 5 μm to about 10 μm, and about 10 μm to about 20 μm.

In an embodiment, fabric is treated with a polymer, such as polyglycolide (PGA), polyethylene glycols, copolymers of glycolide, glycolide/L-lactide copolymers (PGA/PLLA), glycolide/trimethylene carbonate copolymers (PGA/TMC), polylactides (PLA), stereocopolymers of PLA, poly-L-lactide (PLLA), poly-DL-lactide (PDLLA), L-lactide/DL-lactide copolymers, co-polymers of PLA, lactide/tetramethylglycolide copolymers, lactide/trimethylene carbonate copolymers, lactide/6-valerolactone copolymers, lactide/ε-caprolactone copolymers, polydepsipeptides, PLA/polyethylene oxide copolymers, unsymmetrically 3,6-substituted poly-1,4-dioxane-2,5-diones, poly-β-hydroxybutyrate (PHBA), PHBA/p-hydroxyvalerate copolymers (PHBA/HVA), poly-β-hydroxypropionate (PHPA), poly-β-dioxanone (PDS), poly-δ-valerolactone, poly-ε-caprolactone, methylmethacrylate-N-vinyl pyrrolidine copolymers, polyesteramides, polyesters of oxalic acid, polydihydropyrans, polyalkyl-2-cyanoacrylates, polyurethanes (PU), polyvinylalcohols (PVA), polypeptides, poly-β-malic acid (PMLA), poly-β-alkanoic acids, polyvinylalcohol (PVA), polyethyleneoxide (PEO), chitine polymers, polyethylene, polypropylene, polyasetal, polyamides, polyesters, polysulphone, polyether ether ketone, polyethylene terephthalate, polycarbonate, polyaryl ether ketone, and polyether ketone.

In an embodiment, textiles may be manufactured via one or more of the following processes weaving processes, knitting processes, and non-woven processes. In an embodiment, weaving processes may include plain weaving, twill weaving, and/or satin weaving. In an embodiment, knitting processes may include weft knitting (e.g., circular, flat bed, and/or full fashioned) and/or warp knitting (e.g., tricot, Raschel, and/or crochet). In an embodiment, non-woven processes may include stable fiber (e.g., dry laid and/or wet laid) and/or continuous filament (e.g., spun laid and/or melt blown).

In an embodiment, the disclosure provides an article comprising a fabric coated with silk protein fragments. In an embodiment, the disclosure provides an article comprising a fabric coated with silk protein fragments, wherein the fabric is a fabric used for human apparel, including performance and/or athletic apparel. In an embodiment, the disclosure provides an article comprising a fabric coated with silk protein fragments, and wherein the fabric exhibits improved moisture management properties and/or resistance to microbial growth. In an embodiment, the disclosure provides an article comprising a fabric coated with silk protein fragments, wherein the fabric is a fabric used for home upholstery. In an embodiment, the disclosure provides an article comprising a fabric coated with silk protein fragments, wherein the fabric is used for automobile upholstery. In an embodiment, the disclosure provides an article comprising a fabric coated with silk protein fragments, wherein the fabric is used for aircraft upholstery. In an embodiment, the disclosure provides an article comprising a fabric coated with silk protein fragments, wherein the fabric is used for upholstery in transportation vehicles for public, commercial, military, or other use, including buses and trains. In an embodiment, the disclosure provides an article comprising a fabric coated with silk protein fragments, wherein the fabric is used for upholstery of a product that requires a high degree of resistance to wear as compared to normal upholstery.

In an embodiment, the disclosure provides an article comprising a fabric coated with silk protein fragments, wherein the fabric is a fabric fabricated as trim on automobile upholstery. In an embodiment, the disclosure provides an article comprising a fabric coated with silk protein fragments, wherein the article is a fabric product fabricated as a steering wheel. In an embodiment, the disclosure provides an article comprising a fabric coated with silk protein fragments, wherein the fabric is a fabric product fabricated as a headrest. In an embodiment, the disclosure provides an article comprising a fabric coated with silk protein fragments, wherein the fabric is a fabric product fabricated as an armrest. In an embodiment, the disclosure provides an article comprising a fabric coated with silk protein fragments, wherein the fabric is a fabric product fabricated as an automobile floor mat. In an embodiment, the disclosure provides an article comprising a fabric coated with silk protein fragments, wherein the fabric is a fabric product fabricated as automobile or vehicle carpet. In an embodiment, the disclosure provides an article comprising a fabric coated with silk protein fragments, wherein the fabric is a fabric product fabricated as automotive trim. In an embodiment, the disclosure provides an article comprising a fabric coated with silk protein fragments, wherein the fabric is a fabric product fabricated as a children's car seat. In an embodiment, the disclosure provides an article comprising a fabric coated with silk protein fragments, wherein the fabric is a fabric product fabricated as a seat belt or safety harness. In an embodiment, the disclosure provides an article comprising a fabric coated with silk protein fragments, wherein the fabric is a fabric product fabricated as a dashboard. In an embodiment, the disclosure provides an article comprising a fabric coated with silk protein fragments, wherein the fabric is a fabric product fabricated as a seat. In an embodiment, the disclosure provides an article comprising a fabric coated with silk protein fragments, wherein the fabric is a fabric product fabricated as a seat panel. In an embodiment, the disclosure provides an article comprising a fabric coated with silk protein fragments, wherein the fabric is a fabric product fabricated as an interior panel. In an embodiment, the disclosure provides an article comprising a fabric coated with silk protein fragments, wherein the fabric is a fabric product fabricated as an airbag cover. In an embodiment, the disclosure provides an article comprising a fabric coated with silk protein fragments, wherein the fabric is a fabric product fabricated as an airbag. In an embodiment, the disclosure provides an article comprising a fabric coated with silk protein fragments, wherein the fabric is a fabric product fabricated as a sun visor. In an embodiment, the disclosure provides an article comprising a fabric coated with silk protein fragments, wherein the fabric is a fabric product fabricated as a wiring harness. In an embodiment, the disclosure provides an article coated with silk protein fragments, wherein the article is a cushion. In an embodiment, the disclosure provides an article coated with silk protein fragments, wherein the product is automotive, aircraft, or other vehicular insulation. In some embodiments, the coating comprises an article coated with silk protein fragments, thereof having a weight average molecular weight range of about 1 kDa to about 350 kDa, wherein the silk protein fragments have an average weight average molecular weight range selected from the group consisting of about 5 to about 10 kDa, about 6 kDa to about 17 kDa, about 17 kDa to about 39 kDa, about 39 kDa to about 80 kDa, about 60 to about 100 kDa, and about 80 kDa to about 144 kDa, wherein the silk protein fragments have a polydispersity of between about 1.5 and about 3.0, or about 1.0 and about 5.0, and optionally wherein the proteins or protein fragments, prior to coating the fabric, do not spontaneously or gradually gelate and do not visibly change in color or turbidity when in a solution for at least 10 days. The coating comprises silk protein fragments having a weight average molecular weight range of about 5 kDa and about 144 kDa, wherein the silk protein fragments have an average weight average molecular weight range selected from the group consisting of about 5 to about 10 kDa, about 6 kDa to about 17 kDa, about 17 kDa to about 39 kDa, about 39 kDa to about 80 kDa, about 60 to about 100 kDa, and about 80 kDa to about 144 kDa, wherein the silk protein fragments have a polydispersity of between about 1.5 and about 3.0, and optionally wherein the proteins or protein fragments, prior to coating the fabric, do not spontaneously or gradually gelate and do not visibly change in color or turbidity when in a solution for at least 10 days.

In an embodiment, the disclosure provides an article comprising fabric coated with silk protein fragments. In an embodiment, the article is a fabric used in the manufacture of tents, sleeping bags, ponchos, and soft-walled coolers. In an embodiment, the fabric is a fabric used in the manufacture of athletic equipment. In an embodiment, the fabric is a fabric used in the manufacture of outdoor gear. In an embodiment, the fabric is a fabric used in the manufacture of hiking gear, such as harnesses and backpacks. In an embodiment, the fabric is a fabric used in the manufacture of climbing gear. In an embodiment, the fabric is canvas. In an embodiment, the fabric is a fabric used in the manufacture of a hat. In an embodiment, the fabric is a fabric used in the manufacture of an umbrella. In an embodiment, the fabric is a fabric used in the manufacture of a tent. In an embodiment, the fabric is a fabric used in the manufacture of a baby sleeper, a baby blanket, or a baby pajama. In an embodiment, the fabric is a fabric used in the manufacture of a glove, such as a driving glove or an athletic glove. In an embodiment, the fabric is a fabric used in the manufacture of athletic pants, such as sweat pants, jogging pants, yoga pants, or pants for use in competitive sports. In an embodiment, the fabric is a fabric used in the manufacture of athletic shirts, such as sweat shirts, jogging shirts, yoga shirts, or shirts for use in competitive sports. In an embodiment, the fabric is a fabric used in the manufacture of beach equipment, such as beach umbrellas, beach chairs, beach blankets, and beach towels. In an embodiment, the fabric is a fabric used in the manufacture of jackets or overcoats. In an embodiment, the fabric is a fabric used in the manufacture of medical garments, such as surgical drapes, surgical gowns, surgical sleeves, laboratory sleeves, laboratory coats, wound dressings, sterilization wraps, surgical face masks, retention bandages, support devices, compression bandages, shoe covers, surgical blankets, and the like. The coating comprises silk based proteins or fragments thereof having a weight average molecular weight range of about 5 kDa to about 144 kDa.

In an embodiment, the disclosure provides an article comprising a textile coated with silk fibroin-based proteins or fragments thereof. In an embodiment, the textile is a textile used in the manufacture of tents, sleeping bags, ponchos, and soft-walled coolers. In an embodiment, the textile is a textile used in the manufacture of athletic equipment. In an embodiment, the textile is a textile used in the manufacture of outdoor gear. In an embodiment, the textile is a textile used in the manufacture of hiking gear, such as harnesses and backpacks. In an embodiment, the textile is a textile used in the manufacture of climbing gear. In an embodiment, the textile is canvas. In an embodiment, the textile is a textile used in the manufacture of a hat. In an embodiment, the textile is a textile used in the manufacture of an umbrella. In an embodiment, the textile is a textile used in the manufacture of a tent. In an embodiment, the textile is a textile used in the manufacture of a baby sleeper, a baby blanket, or a baby pajama. In an embodiment, the textile is a textile used in the manufacture of a glove, such as a driving glove or an athletic glove. In an embodiment, the textile is a textile used in the manufacture of athletic pants, such as sweat pants, jogging pants, yoga pants, or pants for use in competitive sports. In an embodiment, the textile is a textile used in the manufacture of athletic shirts, such as sweat shirts, jogging shirts, yoga shirts, or shirts for use in competitive sports. In an embodiment, the textile is a textile used in the manufacture of beach equipment, such as beach umbrellas, beach chairs, beach blankets, and beach towels. In an embodiment, the textile is a textile used in the manufacture of jackets or overcoats. In an embodiment, the textile is a textile used in the manufacture of medical garments, such as surgical drapes, surgical gowns, surgical sleeves, laboratory sleeves, laboratory coats, wound dressings, sterilization wraps, surgical face masks, retention bandages, support devices, compression bandages, shoe covers, surgical blankets, and the like. The coating comprises silk based proteins or fragments thereof having a weight average molecular weight range of about 1 kDa to about 350 kDa, wherein the silk based proteins or protein fragments thereof have an average weight average molecular weight range selected from the group consisting of about 5 to about 10 kDa, about 6 kDa to about 17 kDa, about 17 kDa to about 39 kDa, about 39 kDa to about 80 kDa, about 60 to about 100 kDa, and about 80 kDa to about 144 kDa, wherein the silk based proteins or fragments thereof have a polydispersity of between about 1.0 and about 5.0, and optionally wherein the proteins or protein fragments, prior to coating the fabric, do not spontaneously or gradually gelate and do not visibly change in color or turbidity when in a solution for at least 10 days. In an embodiment, the disclosure provides a shoe coated with silk fibroin-based proteins or fragments thereof. In an embodiment, the disclosure provides a shoe coated with silk fibroin-based proteins or fragments thereof, wherein the shoe exhibits an improved property relative to an uncoated shoe. In an embodiment, the disclosure provides a shoe coated with silk fibroin-based proteins or fragments thereof, wherein the shoe exhibits an improved property relative to an uncoated shoe, and wherein the improved property is stain resistance. In an embodiment, the disclosure provides a shoe coated with silk fibroin-based proteins or fragments thereof, wherein the shoe exhibits an improved property relative to an uncoated shoe, and wherein the shoe is made of natural leather or synthetic leather. The coating comprises silk based proteins or fragments thereof having a weight average molecular weight range of about 1 kDa to about 350 kDa, or about 5 kDa to about 144 kDa, wherein the silk based proteins or protein fragments thereof have an average weight average molecular weight range selected from the group consisting of about 5 to about 10 kDa, about 6 kDa to about 17 kDa, about 17 kDa to about 39 kDa, about 39 kDa to about 80 kDa, about 60 to about 100 kDa, and about 80 kDa to about 144 kDa, wherein the silk based proteins or fragments thereof have a polydispersity of between about 1.0 and about 5.0, or about 1.5 and about 3.0, and optionally wherein the proteins or protein fragments, prior to coating the fabric, do not spontaneously or gradually gelate and do not visibly change in color or turbidity when in a solution for at least 10 days.

In one aspect, the disclosure provides methods of making a silk coated fabric and/or articles using the silk protein fragments of the disclosure. In some embodiments, the silk coated fabric is a silk fibroin coated fabric. In some embodiments, the silk coated article is a silk fibroin coated article. In some embodiments, the disclosure also includes an article prepared by the methods of the disclosure. In some embodiments, the disclosure also includes an article comprising a coated fabric prepared by the methods of the disclosure. In some embodiments, the disclosure also includes a coated fabric prepared by the methods of the disclosure.

In some embodiments, the disclosure includes a method of making a silk fibroin coated fabric, comprising applying to the fabric a solution comprising a reducing agent, applying to the fabric a silk fibroin solution, and drying the fabric.

In some embodiments, the disclosure includes a method of improving size retention on laundering in a fabric, comprising applying to the fabric a solution comprising a reducing agent, applying to the fabric a silk fibroin solution, and drying the fabric.

In one aspect, the disclosure includes a method of improving size retention on laundering in a fabric comprising coating a surface of the fabric with a solution comprising a reducing agent, preparing a silk fibroin solution comprising silk protein fibroin fragments, coating a surface of the fabric with the silk fibroin solution, and drying the surface of the fabric that has been coated with the silk fibroin solution, wherein upon laundering, the coated fabric substantially retains its initial size prior to laundering.

Any surfactant and/or emulsifier is contemplated by the present disclosure. In a non-limiting example, the surfactant and/or emulsifier is used as mixed with a silk fibroin solution to treat the fabric. In a non-limiting example, the surfactant and/or emulsifier is used to pretreat the surface of the fabric in order to improve the surface affinity between the silk protein fragments and the fabric. In some embodiments, the surfactant and/or emulsifier is a natural surfactant and/or emulsifier. In some embodiments, the surfactant and/or emulsifier is selected from coco glucoside, decyl glucoside, lauryl glucoside, sucrose cocoate, capryl/caprylyl glucoside, and caprylyl/capryl glucoside. In some embodiments, the surfactant and/or emulsifier is selected from Polyoxyethylene sorbitan monooleate, Polyoxyethylene sorbitan trioleate, and Polyoxyethylene castor oil. In some embodiments, the surfactant and/or emulsifier is selected from Polyoxyethylene (10-30) sorbitan monooleate, Polyoxyethylene (10-30) sorbitan trioleate, and Polyoxyethylene (10-50) castor oil. In some embodiments, the surfactant and/or emulsifier is selected from Polyoxyethylene (20) sorbitan monooleate, Polyoxyethylene (20) sorbitan trioleate, and Polyoxyethylene (29) castor oil. In some embodiments, the surfactant and/or emulsifier is Polyoxyethylene (20) sorbitan monooleate. In some embodiments, the surfactant and/or emulsifier is Polyoxyethylene (20) sorbitan monolaurate. In some embodiments, the surfactant and/or emulsifier is Polyoxyethylene (20) sorbitan monopalmitate. In some embodiments, the surfactant and/or emulsifier is Polyoxyethylene (20) sorbitan monostearate. In some embodiments, the surfactant and/or emulsifier is Polyoxyethylene (20) sorbitan trioleate. In some embodiments, the surfactant and/or emulsifier is Polyoxyethylene (20) sorbitan tristearate. In some embodiments, the surfactant and/or emulsifier is Polyoxyethylene (29) castor oil. In some embodiments, the surfactant and/or emulsifier comprises a sorbitan mono fatty acid. In some embodiments, the surfactant and/or emulsifier comprises a sorbitan tri fatty acid. In some embodiments, the surfactant and/or emulsifier comprises a castor oil. In some embodiments, the surfactant and/or emulsifier comprises has a given degree of ethoxylation which can be tuned to result in a specific HLB value. In some embodiments, the concentration of the surfactant and/or emulsifier in the solution ranges from 0.01 g/L to about 100 g/L. In some embodiments, the concentration of the surfactant and/or emulsifier in the solution ranges from 0.1 g/L to about 50 g/L. In some embodiments, the concentration of the surfactant and/or emulsifier in the solution ranges from 0.5 g/L to about 25 g/L. In some embodiments, the concentration of the surfactant and/or emulsifier in the solution ranges from 1 g/L to about 20 g/L. In some embodiments, the concentration of the surfactant and/or emulsifier in the solution ranges from about 20 g/L to about 50 g/L. In some embodiments, the concentration of the surfactant and/or emulsifier is about 1 g/L. In some embodiments, the concentration of the surfactant and/or emulsifier is about 2 g/L. In some embodiments, the concentration of the surfactant and/or emulsifier is about 3 g/L. In some embodiments, the concentration of the surfactant and/or emulsifier is about 4 g/L. In some embodiments, the concentration of the surfactant and/or emulsifier is about 5 g/L. In some embodiments, the concentration of the surfactant and/or emulsifier is about 6 g/L. In some embodiments, the concentration of the surfactant and/or emulsifier is about 7 g/L. In some embodiments, the concentration of the surfactant and/or emulsifier is about 8 g/L. In some embodiments, the concentration of the surfactant and/or emulsifier is about 9 g/L. In some embodiments, the concentration of the surfactant and/or emulsifier is about 10 g/L. In some embodiments, the concentration of the surfactant and/or emulsifier is about 11 g/L. In some embodiments, the concentration of the surfactant and/or emulsifier is about 12 g/L. In some embodiments, the concentration of the surfactant and/or emulsifier is about 13 g/L. In some embodiments, the concentration of the surfactant and/or emulsifier is about 14 g/L. In some embodiments, the concentration of the surfactant and/or emulsifier is about 15 g/L. In some embodiments, the concentration of the surfactant and/or emulsifier is about 16 g/L. In some embodiments, the concentration of the surfactant and/or emulsifier is about 17 g/L. In some embodiments, the concentration of the surfactant and/or emulsifier is about 18 g/L. In some embodiments, the concentration of the surfactant and/or emulsifier is about 19 g/L. In some embodiments, the concentration of the surfactant and/or emulsifier is about 20 g/L. In some embodiments, the concentration of the surfactant and/or emulsifier is about 21 g/L. In some embodiments, the concentration of the surfactant and/or emulsifier is about 22 g/L. In some embodiments, the concentration of the surfactant and/or emulsifier is about 23 g/L. In some embodiments, the concentration of the surfactant and/or emulsifier is about 24 g/L. In some embodiments, the concentration of the surfactant and/or emulsifier is about 25 g/L. In some embodiments, the concentration of the surfactant and/or emulsifier is about 26 g/L. In some embodiments, the concentration of the surfactant and/or emulsifier is about 27 g/L. In some embodiments, the concentration of the surfactant and/or emulsifier is about 28 g/L. In some embodiments, the concentration of the surfactant and/or emulsifier is about 29 g/L. In some embodiments, the concentration of the surfactant and/or emulsifier is about 30 g/L. In some embodiments, the concentration of the surfactant and/or emulsifier is about 31 g/L. In some embodiments, the concentration of the surfactant and/or emulsifier is about 32 g/L. In some embodiments, the concentration of the surfactant and/or emulsifier is about 33 g/L. In some embodiments, the concentration of the surfactant and/or emulsifier is about 34 g/L. In some embodiments, the concentration of the surfactant and/or emulsifier is about 35 g/L. In some embodiments, the concentration of the surfactant and/or emulsifier is about 36 g/L. In some embodiments, the concentration of the surfactant and/or emulsifier is about 37 g/L. In some embodiments, the concentration of the surfactant and/or emulsifier is about 38 g/L. In some embodiments, the concentration of the surfactant and/or emulsifier is about 39 g/L. In some embodiments, the concentration of the surfactant and/or emulsifier is about 40 g/L. In some embodiments, the concentration of the surfactant and/or emulsifier is about 41 g/L. In some embodiments, the concentration of the surfactant and/or emulsifier is about 42 g/L. In some embodiments, the concentration of the surfactant and/or emulsifier is about 43 g/L. In some embodiments, the concentration of the surfactant and/or emulsifier is about 44 g/L. In some embodiments, the concentration of the surfactant and/or emulsifier is about 45 g/L. In some embodiments, the concentration of the surfactant and/or emulsifier is about 46 g/L. In some embodiments, the concentration of the surfactant and/or emulsifier is about 47 g/L. In some embodiments, the concentration of the surfactant and/or emulsifier is about 48 g/L. In some embodiments, the concentration of the surfactant and/or emulsifier is about 49 g/L. In some embodiments, the concentration of the surfactant and/or emulsifier is about 50 g/L.

In some embodiments, the concentration of the silk fibroin fragments in the solution ranges from 0.01 g/L to about 100 g/L. In some embodiments, the concentration of the silk fibroin fragments in the solution ranges from 0.1 g/L to about 50 g/L. In some embodiments, the concentration of the silk fibroin fragments in the solution ranges from 0.5 g/L to about 25 g/L. In some embodiments, the concentration of the silk fibroin fragments in the solution ranges from 1 g/L to about 20 g/L. In some embodiments, the concentration of the silk fibroin fragments in the solution ranges from about 20 g/L to about 50 g/L. In some embodiments, the concentration of the silk fibroin fragments is about 1 g/L. In some embodiments, the concentration of the silk fibroin fragments is about 2 g/L. In some embodiments, the concentration of the silk fibroin fragments is about 3 g/L. In some embodiments, the concentration of the silk fibroin fragments is about 4 g/L. In some embodiments, the concentration of the silk fibroin fragments is about 5 g/L. In some embodiments, the concentration of the silk fibroin fragments is about 6 g/L. In some embodiments, the concentration of the silk fibroin fragments is about 7 g/L. In some embodiments, the concentration of the silk fibroin fragments is about 8 g/L. In some embodiments, the concentration of the silk fibroin fragments is about 9 g/L. In some embodiments, the concentration of the silk fibroin fragments is about 10 g/L. In some embodiments, the concentration of the silk fibroin fragments is about 11 g/L. In some embodiments, the concentration of the silk fibroin fragments is about 12 g/L. In some embodiments, the concentration of the silk fibroin fragments is about 13 g/L. In some embodiments, the concentration of the silk fibroin fragments is about 14 g/L. In some embodiments, the concentration of the silk fibroin fragments is about 15 g/L. In some embodiments, the concentration of the silk fibroin fragments is about 16 g/L. In some embodiments, the concentration of the silk fibroin fragments is about 17 g/L. In some embodiments, the concentration of the silk fibroin fragments is about 18 g/L. In some embodiments, the concentration of the silk fibroin fragments is about 19 g/L. In some embodiments, the concentration of the silk fibroin fragments is about 20 g/L. In some embodiments, the concentration of the silk fibroin fragments is about 21 g/L. In some embodiments, the concentration of the silk fibroin fragments is about 22 g/L. In some embodiments, the concentration of the silk fibroin fragments is about 23 g/L. In some embodiments, the concentration of the silk fibroin fragments is about 24 g/L. In some embodiments, the concentration of the silk fibroin fragments is about 25 g/L. In some embodiments, the concentration of the silk fibroin fragments is about 26 g/L. In some embodiments, the concentration of the silk fibroin fragments is about 27 g/L. In some embodiments, the concentration of the silk fibroin fragments is about 28 g/L. In some embodiments, the concentration of the silk fibroin fragments is about 29 g/L. In some embodiments, the concentration of the silk fibroin fragments is about 30 g/L. In some embodiments, the concentration of the silk fibroin fragments is about 31 g/L. In some embodiments, the concentration of the silk fibroin fragments is about 32 g/L. In some embodiments, the concentration of the silk fibroin fragments is about 33 g/L. In some embodiments, the concentration of the silk fibroin fragments is about 34 g/L. In some embodiments, the concentration of the silk fibroin fragments is about 35 g/L. In some embodiments, the concentration of the silk fibroin fragments is about 36 g/L. In some embodiments, the concentration of the silk fibroin fragments is about 37 g/L. In some embodiments, the concentration of the silk fibroin fragments is about 38 g/L. In some embodiments, the concentration of the silk fibroin fragments is about 39 g/L. In some embodiments, the concentration of the silk fibroin fragments is about 40 g/L. In some embodiments, the concentration of the silk fibroin fragments is about 41 g/L. In some embodiments, the concentration of the silk fibroin fragments is about 42 g/L. In some embodiments, the concentration of the silk fibroin fragments is about 43 g/L. In some embodiments, the concentration of the silk fibroin fragments is about 44 g/L. In some embodiments, the concentration of the silk fibroin fragments is about 45 g/L. In some embodiments, the concentration of the silk fibroin fragments is about 46 g/L. In some embodiments, the concentration of the silk fibroin fragments is about 47 g/L. In some embodiments, the concentration of the silk fibroin fragments is about 48 g/L. In some embodiments, the concentration of the silk fibroin fragments is about 49 g/L. In some embodiments, the concentration of the silk fibroin fragments is about 50 g/L.

In some embodiments, the w/w ratio of silk fibroin fragments to surfactant and/or emulsifier in the solution is about 99:1, about 98:2, about 97:3, about 96:4, about 95:5, about 94:6, about 93:7, about 92:8, about 91:9, about 90:10, about 89:11, about 88:12, about 87:13, about 86:14, about 85:15, about 84:16, about 83:17, about 82:18, about 81:19, about 80:20, about 79:21, about 78:22, about 77:23, about 76:24, about 75:25, about 74:26, about 73:27, about 72:28, about 71:29, about 70:30, about 69:31, about 68:32, about 67:33, about 66:34, about 65:35, about 64:36, about 63:37, about 62:38, about 61:39, about 60:40, about 59:41, about 58:42, about 57:43, about 56:44, about 55:45, about 54:46, about 53:47, about 52:48, about 51:49, about 50:50, about 49:51, about 48:52, about 47:53, about 46:54, about 45:55, about 44:56, about 43:57, about 42:58, about 41:59, about 40:60, about 39:61, about 38:62, about 37:63, about 36:64, about 35:65, about 34:66, about 33:67, about 32:68, about 31:69, about 30:70, about 29:71, about 28:72, about 27:73, about 26:74, about 25:75, about 24:76, about 23:77, about 22:78, about 21:79, about 20:80, about 19:81, about 18:82, about 17:83, about 16:84, about 15:85, about 14:86, about 13:87, about 12:88, about 11:89, about 10:90, about 9:91, about 8:92, about 7:93, about 6:94, about 5:95, about 4:96, about 3:97, about 2:98, or about 1:99. In some embodiments, the w/w ratio of silk fibroin fragments to surfactant and/or emulsifier in the solution is about 1:1.

In some embodiments, the w/w ratio of silk fibroin fragments to surfactant and/or emulsifier in the article is about 99:1, about 98:2, about 97:3, about 96:4, about 95:5, about 94:6, about 93:7, about 92:8, about 91:9, about 90:10, about 89:11, about 88:12, about 87:13, about 86:14, about 85:15, about 84:16, about 83:17, about 82:18, about 81:19, about 80:20, about 79:21, about 78:22, about 77:23, about 76:24, about 75:25, about 74:26, about 73:27, about 72:28, about 71:29, about 70:30, about 69:31, about 68:32, about 67:33, about 66:34, about 65:35, about 64:36, about 63:37, about 62:38, about 61:39, about 60:40, about 59:41, about 58:42, about 57:43, about 56:44, about 55:45, about 54:46, about 53:47, about 52:48, about 51:49, about 50:50, about 49:51, about 48:52, about 47:53, about 46:54, about 45:55, about 44:56, about 43:57, about 42:58, about 41:59, about 40:60, about 39:61, about 38:62, about 37:63, about 36:64, about 35:65, about 34:66, about 33:67, about 32:68, about 31:69, about 30:70, about 29:71, about 28:72, about 27:73, about 26:74, about 25:75, about 24:76, about 23:77, about 22:78, about 21:79, about 20:80, about 19:81, about 18:82, about 17:83, about 16:84, about 15:85, about 14:86, about 13:87, about 12:88, about 11:89, about 10:90, about 9:91, about 8:92, about 7:93, about 6:94, about 5:95, about 4:96, about 3:97, about 2:98, or about 1:99. In some embodiments, the w/w ratio of silk fibroin fragments to surfactant and/or emulsifier in the article is about 1:1.

In some embodiments, the silk fibroin solution comprises low molecular weight silk fibroin-based protein fragments, medium molecular weight silk fibroin-based protein fragments, and/or high molecular weight silk fibroin-based protein fragments. In some embodiments, the silk fibroin solution comprises low molecular weight silk fibroin-based protein fragments. In some embodiments, the silk fibroin solution comprises medium molecular weight silk fibroin-based protein fragments. In some embodiments, drying the surface of the fabric comprises heating the surface of the fabric without substantially modifying silk fibroin coating performance. In some embodiments, the method includes an additional step of drying the surface of the fabric. In some embodiments, the additional drying step is performed after coating a surface of the fabric with the solution comprising a reducing agent. In some embodiments, the additional drying step is performed before coating the surface with the silk fibroin solution.

In some embodiments, upon laundering, the fabric substantially retains its initial size prior to laundering. In some embodiments, upon laundering, the fabric retains a substantially higher fraction of its initial size prior to laundering compared to a similar fabric not similarly treated with the surfactant and/or emulsifier and the silk fibroin solution

In any of the foregoing embodiments, at least one property of the article is improved, wherein the property that is improved is dimensional stability to laundering, and wherein the property is improved by an amount relative to an uncoated article selected from the group consisting of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 200%, at least 300%, at least 400%, and at least 500%. In any of the foregoing embodiments, at least one property of the article is improved, wherein the property that is improved is moisture management. In some embodiments, moisture management is improved comparative to a similar article comprising a similar fabric but no coating. Moisture management can be assessed by any method known in the art, for example, and without limitation, by a water absorbency test, a vertical wicking test, or a dry rate test. Moisture management can be improved by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 200%, at least 300%, at least 400%, or at least 500%.

In any of the foregoing embodiments, at least one property of the article is improved, wherein the property that is improved is size retention on laundering, and wherein the property is improved by an amount relative to an uncoated article selected from the group consisting of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 200%, at least 300%, at least 400%, and at least 500%.

In any of the foregoing embodiments, at least one property of the article is improved, wherein the property that is improved is resistance to shrinkage, and wherein the property is improved by an amount relative to an uncoated article selected from the group consisting of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 200%, at least 300%, at least 400%, and at least 500%.

In an embodiment, the foregoing improved property, or any other improved property described herein, is determined after a period of machine washing (e.g., by home laundering machine washing) cycles selected from the group consisting of 0 cycles, 1 cycle, 2 cycles, 3 cycles, 4 cycles, 5 cycles, 6 cycles, 7 cycles, 8 cycles, 9 cycles, 10 cycles, 11 cycles, 12 cycles, 13 cycles, 14 cycles, 15 cycles, 20 cycles, 25 cycles, 30 cycles, 35 cycles, 40 cycles, 45 cycles, and 50 cycles.

In an embodiment, the concentration of the silk fibroin solution is less than 30.0% w/v. In an embodiment, the concentration of the silk fibroin solution is less than 25.0% w/v. In an embodiment, the concentration of the silk fibroin solution is less than 20.0% w/v. In an embodiment, the concentration of the silk fibroin solution is less than 19.0% w/v. In an embodiment, the concentration of the silk fibroin solution is less than 18.0% w/v. In an embodiment, the concentration of the silk fibroin solution is less than 17.0% w/v. In an embodiment, the concentration of the silk fibroin solution is less than 16.0% w/v. In an embodiment, the concentration of the silk fibroin solution is less than 15.0% w/v. In an embodiment, the concentration of the silk fibroin solution is less than 14.0% w/v. In an embodiment, the concentration of the silk fibroin solution is less than 13.0% w/v. In an embodiment, the concentration of the silk fibroin solution is less than 12.0% w/v. In an embodiment, the concentration of the silk fibroin solution is less than 11.0% w/v. In an embodiment, the concentration of the silk fibroin solution is less than 10.0% w/v. In an embodiment, the concentration of the silk fibroin solution is less than 9.0% w/v. In an embodiment, the concentration of the silk fibroin solution is less than 8.0% w/v. In an embodiment, the concentration of the silk fibroin solution is less than 7.0% w/v. In an embodiment, the concentration of the silk fibroin solution is less than 6.0% w/v. In an embodiment, the concentration of the silk fibroin solution is less than 5.0% w/v. In an embodiment, the concentration of the silk fibroin solution is less than 4.0% w/v. In an embodiment, the concentration of the silk fibroin solution is less than 3.0% w/v. In an embodiment, the concentration of the silk fibroin solution is less than 2.0% w/v. In an embodiment, the concentration of the silk fibroin solution is less than 1.0% w/v. In an embodiment, the concentration of the silk fibroin solution is less than 0.9% w/v. In an embodiment, the concentration of the silk fibroin solution is less than 0.8% w/v. In an embodiment, the concentration of the silk fibroin solution is less than 0.7% w/v. In an embodiment, the concentration of the silk fibroin solution is less than 0.6% w/v. In an embodiment, the concentration of the silk fibroin solution is less than 0.5% w/v. In an embodiment, the concentration of the silk fibroin solution is less than 0.4% w/v. In an embodiment, the concentration of the silk fibroin solution is less than 0.3% w/v. In an embodiment, the concentration of the silk fibroin solution is less than 0.2% w/v.

In an embodiment, the concentration of the silk fibroin solution is less than 0.1% w/v. In an embodiment, the concentration of the silk fibroin solution is greater than 0.1% w/v. In an embodiment, the concentration of the silk fibroin solution is greater than 0.2% w/v. In an embodiment, the concentration of the silk fibroin solution is greater than 0.3% w/v. In an embodiment, the concentration of the silk fibroin solution is greater than 0.4% w/v. In an embodiment, the concentration of the silk fibroin solution is greater than 0.5% w/v. In an embodiment, the concentration of the silk fibroin solution is greater than 0.6% w/v. In an embodiment, the concentration of the silk fibroin solution is greater than 0.7% w/v. In an embodiment, the concentration of the silk fibroin solution is greater than 0.8% w/v. In an embodiment, the concentration of the silk fibroin solution is greater than 0.9% w/v. In an embodiment, the concentration of the silk fibroin solution is greater than 1.0% w/v. In an embodiment, the concentration of the silk fibroin solution is greater than 2.0% w/v. In an embodiment, the concentration of the silk fibroin solution is greater than 3.0% w/v. In an embodiment, the concentration of the silk fibroin solution is greater than 4.0% w/v. In an embodiment, the concentration of the silk fibroin solution is greater than 5.0% w/v. In an embodiment, the concentration of the silk fibroin solution is greater than 6.0% w/v. In an embodiment, the concentration of the silk fibroin solution is greater than 7.0% w/v. In an embodiment, the concentration of the silk fibroin solution is greater than 8.0% w/v. In an embodiment, the concentration of the silk fibroin solution is greater than 9.0% w/v. In an embodiment, the concentration of the silk fibroin solution is greater than 10.0% w/v. In an embodiment, the concentration of the silk fibroin solution is greater than 11.0% w/v. In an embodiment, the concentration of the silk fibroin solution is greater than 120.0% w/v. In an embodiment, the concentration of the silk fibroin solution is greater than 130.0% w/v. In an embodiment, the concentration of the silk fibroin solution is greater than 140.0% w/v. In an embodiment, the concentration of the silk fibroin solution is greater than 15.0% w/v. In an embodiment, the concentration of the silk fibroin solution is greater than 160.0% w/v. In an embodiment, the concentration of the silk fibroin solution is greater than 17.0% w/v. In an embodiment, the concentration of the silk fibroin solution is greater than 18.0% w/v. In an embodiment, the concentration of the silk fibroin solution is greater than 190.0% w/v. In an embodiment, the concentration of the silk fibroin solution is greater than 20.0% w/v. In an embodiment, the concentration of the silk fibroin solution is greater than 25.0% w/v.

In an embodiment, the concentration of the silk fibroin solution ranges from about 0.1% w/v to about 30.0% w/v. In an embodiment, the concentration of the silk fibroin solution ranges from about 0.1% w/v to about 25.0% w/v. In an embodiment, the concentration of the silk fibroin solution ranges from about 0.1% w/v to about 20.0% w/v. In an embodiment, the concentration of the silk fibroin solution ranges from about 0.1% w/v to about 15.0% w/v. In an embodiment, the concentration of the silk fibroin solution ranges from about 0.1% w/v to about 10.0% w/v. In an embodiment, the concentration of the silk fibroin solution ranges from about 0.1% w/v to about 9.0% w/v. In an embodiment, the concentration of the silk fibroin solution ranges from about 0.1% w/v to about 8.0% w/v. In an embodiment, the concentration of the silk fibroin solution ranges from about 0.1% w/v to about 7.0% w/v. In an embodiment, the concentration of the silk fibroin solution ranges from about 0.1% w/v to about 6.5% w/v. In an embodiment, the concentration of the silk fibroin solution ranges from about 0.1% w/v to about 6.0% w/v. In an embodiment, the concentration of the silk fibroin solution ranges from about 0.1% w/v to about 5.5% w/v. In an embodiment, the concentration of the silk fibroin solution ranges from about 0.1% w/v to about 5.0% w/v. In an embodiment, the concentration of the silk fibroin solution ranges from about 0.1% w/v to about 4.5% w/v. In an embodiment, the concentration of the silk fibroin solution ranges from about 0.1% w/v to about 4.0% w/v. In an embodiment, the concentration of the silk fibroin solution ranges from about 0.1% w/v to about 3.5% w/v. In an embodiment, the concentration of the silk fibroin solution ranges from about 0.1% w/v to about 3.0% w/v. In an embodiment, the concentration of the silk fibroin solution ranges from about 0.1% w/v to about 2.5% w/v. In an embodiment, the concentration of the silk fibroin solution ranges from about 0.1% w/v to about 2.0% w/v. In an embodiment, the concentration of the silk fibroin solution ranges from about 0.1% w/v to about 2.4% w/v. In an embodiment, the concentration of the silk fibroin solution ranges from about 0.5% w/v to about 5.0% w/v. In an embodiment, the concentration of the silk fibroin solution ranges from about 0.5% w/v to about 4.5% w/v. In an embodiment, the concentration of the silk fibroin solution ranges from about 0.5% w/v to about 4.0% w/v. In an embodiment, the concentration of the silk fibroin solution ranges from about 0.5% w/v to about 3.5% w/v. In an embodiment, the concentration of the silk fibroin solution ranges from about 0.5% w/v to about 3.0% w/v. In an embodiment, the concentration of the silk fibroin solution ranges from about 0.5% w/v to about 2.5% w/v. In an embodiment, the concentration of the silk fibroin solution ranges from about 1.0% w/v to about 4.0% w/v. In an embodiment, the concentration of the silk fibroin solution ranges from about 1.0% w/v to about 3.5% w/v. In an embodiment, the concentration of the silk fibroin solution ranges from about 1.0% w/v to about 3.0% w/v. In an embodiment, the concentration of the silk fibroin solution ranges from about 1.0% w/v to about 2.5% w/v. In an embodiment, the concentration of the silk fibroin solution ranges from about 1.0% w/v to about 2.4% w/v. In an embodiment, the concentration of the silk fibroin solution ranges from about 1.0% w/v to about 2.0% w/v.

In an embodiment, the concentration of the silk fibroin solution ranges from about 20.0% w/v to about 30.0% w/v. In an embodiment, the concentration of the silk fibroin solution ranges from about 0.1% w/v to about 10.0% w/v. In an embodiment, the concentration of the silk fibroin solution ranges from about 1.0% w/v to about 10.0% w/v. In an embodiment, the concentration of the silk fibroin solution ranges from about 2% w/v to about 10.0% w/v. In an embodiment, the concentration of the silk fibroin solution ranges from about 0.1% w/v to about 6.0% w/v. In an embodiment, the concentration of the silk fibroin solution ranges from about 6.0% w/v to about 10.0% w/v. In an embodiment, the concentration of the silk fibroin solution ranges from about 6.0% w/v to about 8.0% w/v. In an embodiment, the concentration of the silk fibroin solution ranges from about 6.0% w/v to about 9.0% w/v. In an embodiment, the concentration of the silk fibroin solution ranges from about 10.0% w/v to about 20.0% w/v. In an embodiment, the concentration of the silk fibroin solution ranges from about 11.0% w/v to about 190.0% w/v. In an embodiment, the concentration of the silk fibroin solution ranges from about 120.0% w/v to about 18.0% w/v. In an embodiment, the concentration of the silk fibroin solution ranges from about 130.0% w/v to about 170.0% w/v. In an embodiment, the concentration of the silk fibroin solution ranges from about 14.0% w/v to about 16.0% w/v. In an embodiment, the concentration of the silk fibroin solution is about 1.0% w/v. In an embodiment, the concentration of the silk fibroin solution is about 0.5% w/v. In an embodiment, the concentration of the silk fibroin solution is about 1.5% w/v. In an embodiment, the concentration of the silk fibroin solution is about 2.0 wt. %. In an embodiment, the concentration of the silk fibroin solution is about 2.4% w/v. In an embodiment, the concentration of the silk fibroin solution is 3.0% w/v. In an embodiment, the concentration of the silk fibroin solution is 3.5% w/v. In an embodiment, the concentration of the silk fibroin solution is about 4.0% w/v. In an embodiment, the concentration of the silk fibroin solution is about 4.5% w/v. In an embodiment, the concentration of the silk fibroin solution is about 5.0% w/v. In an embodiment, the concentration of the silk fibroin solution is about 5.5% w/v. In an embodiment the concentration of the silk fibroin solution is about 6.0% w/v. In an embodiment, the concentration of the silk fibroin solution is about 6.5% w/v. In an embodiment, the concentration of the silk fibroin solution is about 7.0% w/v. In an embodiment, the concentration of the silk fibroin solution is about 7.5% w/v. In an embodiment, the concentration of the silk fibroin solution is about 8.0% w/v. In an embodiment, the concentration of the silk fibroin solution is about 8.5% w/v. In an embodiment, the concentration of the silk fibroin solution is about 9.0% w/v. In an embodiment, the concentration of the silk fibroin solution is about 9.5% w/v. In an embodiment, the concentration of the silk fibroin solution is about 10.0% w/v.

In some embodiments, the SFS includes an acidic agent. In some embodiments, an acidic agent is a Bronsted acid. In an embodiment, the acidic agent includes one or more of citric acid and acetic acid. In an embodiment, the acidic agent aids the deposition and coating of SPF mixtures (i.e., SFS coating) on the textile to be coated as compared to the absence of such acidic agent. In an embodiment, the acidic agent improves crystallization of the SPF mixtures at the textile to be coated. In an embodiment, the acidic agent is added at a concentration by weight (% w/w or % w/v) or by volume (v/v) of greater than about 0.001%, or greater than about 0.002%, or greater than about 0.003%, or greater than about 0.004%, or greater than about 0.005%, or greater than about 0.006%, or greater than about 0.007%, or greater than about 0.008%, or greater than about 0.009%, or greater than about 0.01%, or greater than about 0.02%, or greater than about 0.03%, or greater than about 0.04%, or greater than about 0.05%, or greater than about 0.06%, or greater than about 0.07%, or greater than about 0.08%, or greater than about 0.09%, or greater than about 0.1%, or greater than about 0.2%, or greater than about 0.3%, or greater than about 0.4%, or greater than about 0.5%, or greater than about 0.6%, or greater than about 0.7%, or greater than about 0.8%, or greater than about 0.9%, or greater than about 1.0% or greater than about 2.0%, or greater than about 3.0%, or greater than about 4.0%, or greater than about 5.0%.

In an embodiment, the acidic agent is added at a concentration by weight (% w/w or % w/v) or by volume (v/v) of less than about 0.001%, or less than about 0.002%, or less than about 0.003%, or less than about 0.004%, or less than about 0.005%, or less than about 0.006%, or less than about 0.007%, or less than about 0.008%, or less than about 0.009%, or less than about 0.01%, or less than about 0.02%, or less than about 0.03%, or less than about 0.04%, or less than about 0.05%, or less than about 0.06%, or less than about 0.07%, or less than about 0.08%, or less than about 0.09%, or less than about 0.1%, or less than about 0.2%, or less than about 0.3%, or less than about 0.4%, or less than about 0.5%, or less than about 0.6%, or less than about 0.7%, or less than about 0.8%, or less than about 0.9%, or less than about 1.0% or less than about 2.0%, or less than about 3.0%, or less than about 4.0%, or less than about 5.0%.

In some embodiments, SFS may have a pH of less than about 9, or less than about 8.5, or less than about 8, or less than about 7.5, or less than about 7, or less than about 6.5, or less than about 6, or less than about 5.5, or less than about 5, or less than about 4.5, or less than about 4, or greater than about 3.5, or greater than about 4, or greater than about 4.5, or greater than about 5, or greater than about 5.5, or greater than about 6, or greater than about 6.5, or greater than about 7, or greater than about 7.5, or greater than about 8, or greater than about 8.5.

In some embodiments, SFS may include an acidic agent, and may have a pH of less than about 9, or less than about 8.5, or less than about 8, or less than about 7.5, or less than about 7, or less than about 6.5, or less than about 6, or less than about 5.5, or less than about 5, or less than about 4.5, or less than about 4, or greater than about 3.5, or greater than about 4, or greater than about 4.5, or greater than about 5, or greater than about 5.5, or greater than about 6, or greater than about 6.5, or greater than about 7, or greater than about 7.5, or greater than about 8, or greater than about 8.5.

In some embodiments, SFS, with or without a surfactant and/or emulsifier, has a pH ranging from about 3 to 5. In some embodiments, SFS, with or without a surfactant and/or emulsifier, has a pH of about 4. In some embodiments, SFS, with or without a surfactant and/or emulsifier, has a pH of about 4.5. In some embodiments, SFS, with or without a surfactant and/or emulsifier, has a pH of between about 4 and about 4.5.

In some embodiments, SFS may be applied to fibers and/or yarn having a diameter of less than about 100 nm, or less than about 200 nm, or less than about 300 nm, or less than about 400 nm, or less than about 500 nm, or less than about 600 nm, or less than about 700 nm, or less than about 800 nm, or less than about 900 nm, or less than about 1000 nm, or less than about 2 μm, or less than about 5 μm, or less than about 10 μm, or less than about 20 μm, or less than about 30 μm, or less than about 40 μm, or less than about 50 μm, or less than about 60 μm, or less than about 70 μm, or less than about 80 μm, or less than about 90 μm, or less than about 100 μm, or less than about 200 μm, or less than about 300 μm, or less than about 400 μm, or less than about 500 μm, or less than about 600 μm, or less than about 700 μm, or less than about 800 μm, or less than about 900 μm, or less than about 1000 μm, or less than about 2 mm, or less than about 3 mm, or less than about 4 mm, or less than about 5 mm, or less than about 6 mm, or less than about 7 mm, or less than about 8 mm, or less than about 9 mm, or less than about 10 mm, or less than about 20 mm, or less than about 30 mm, or less than about 40 mm, or less than about 50 mm, or less than about 60 mm, or less than about 70 mm, or less than about 80 mm, or less than about 90 mm, or less than about 100 mm, or less than about 200 mm, or less than about 300 mm, or less than about 400 mm, or less than about 500 mm, or less than about 600 mm, or less than about 700 mm, or less than about 800 mm, or less than about 900 mm, or less than about 1000 mm.

In some embodiments, SFS may be applied to fibers and/or yarn having a diameter of greater than about 100 nm, or greater than about 200 nm, or greater than about 300 nm, or greater than about 400 nm, or greater than about 500 nm, or greater than about 600 nm, or greater than about 700 nm, or greater than about 800 nm, or greater than about 900 nm, or greater than about 1000 nm, or greater than about 2 μm, or greater than about 5 μm, or greater than about 10 μm, or greater than about 20 μm, or greater than about 30 μm, or greater than about 40 μm, or greater than about 50 μm, or greater than about 60 μm, or greater than about 70 μm, or greater than about 80 μm, or greater than about 90 μm, or greater than about 100 μm, or greater than about 200 μm, or greater than about 300 μm, or greater than about 400 μm, or greater than about 500 μm, or greater than about 600 μm, or greater than about 700 μm, or greater than about 800 μm, or greater than about 900 μm, or greater than about 1000 μm, or greater than about 2 mm, or greater than about 3 mm, or greater than about 4 mm, or greater than about 5 mm, or greater than about 6 mm, or greater than about 7 mm, or greater than about 8 mm, or greater than about 9 mm, or greater than about 10 mm, or greater than about 20 mm, or greater than about 30 mm, or greater than about 40 mm, or greater than about 50 mm, or greater than about 60 mm, or greater than about 70 mm, or greater than about 80 mm, or greater than about 90 mm, or greater than about 100 mm, or greater than about 200 mm, or greater than about 300 mm, or greater than about 400 mm, or greater than about 500 mm, or greater than about 600 mm, or greater than about 700 mm, or greater than about 800 mm, or greater than about 900 mm, or greater than about 1000 mm.

In some embodiments, SFS may be applied to fibers and/or yarn having a length of less than about 100 nm, or less than about 200 nm, or less than about 300 nm, or less than about 400 nm, or less than about 500 nm, or less than about 600 nm, or less than about 700 nm, or less than about 800 nm, or less than about 900 nm, or less than about 1000 nm, or less than about 2 μm, or less than about 5 μm, or less than about 10 μm, or less than about 20 μm, or less than about 30 μm, or less than about 40 μm, or less than about 50 μm, or less than about 60 μm, or less than about 70 μm, or less than about 80 μm, or less than about 90 μm, or less than about 100 μm, or less than about 200 μm, or less than about 300 μm, or less than about 400 μm, or less than about 500 μm, or less than about 600 μm, or less than about 700 μm, or less than about 800 μm, or less than about 900 μm, or less than about 1000 μm, or less than about 2 mm, or less than about 3 mm, or less than about 4 mm, or less than about 5 mm, or less than about 6 mm, or less than about 7 mm, or less than about 8 mm, or less than about 9 mm, or less than about 10 mm, or less than about 20 mm, or less than about 30 mm, or less than about 40 mm, or less than about 50 mm, or less than about 60 mm, or less than about 70 mm, or less than about 80 mm, or less than about 90 mm, or less than about 100 mm, or less than about 200 mm, or less than about 300 mm, or less than about 400 mm, or less than about 500 mm, or less than about 600 mm, or less than about 700 mm, or less than about 800 mm, or less than about 900 mm, or less than about 1000 mm.

In some embodiments, SFS may be applied to fibers and/or yarn having a length of greater than about 100 nm, or greater than about 200 nm, or greater than about 300 nm, or greater than about 400 nm, or greater than about 500 nm, or greater than about 600 nm, or greater than about 700 nm, or greater than about 800 nm, or greater than about 900 nm, or greater than about 1000 nm, or greater than about 2 μm, or greater than about 5 μm, or greater than about 10 μm, or greater than about 20 μm, or greater than about 30 μm, or greater than about 40 μm, or greater than about 50 μm, or greater than about 60 μm, or greater than about 70 μm, or greater than about 80 μm, or greater than about 90 μm, or greater than about 100 μm, or greater than about 200 μm, or greater than about 300 μm, or greater than about 400 μm, or greater than about 500 μm, or greater than about 600 μm, or greater than about 700 μm, or greater than about 800 μm, or greater than about 900 μm, or greater than about 1000 μm, or greater than about 2 mm, or greater than about 3 mm, or greater than about 4 mm, or greater than about 5 mm, or greater than about 6 mm, or greater than about 7 mm, or greater than about 8 mm, or greater than about 9 mm, or greater than about 10 mm, or greater than about 20 mm, or greater than about 30 mm, or greater than about 40 mm, or greater than about 50 mm, or greater than about 60 mm, or greater than about 70 mm, or greater than about 80 mm, or greater than about 90 mm, or greater than about 100 mm, or greater than about 200 mm, or greater than about 300 mm, or greater than about 400 mm, or greater than about 500 mm, or greater than about 600 mm, or greater than about 700 mm, or greater than about 800 mm, or greater than about 900 mm, or greater than about 1000 mm.

In some embodiments, SFS may be applied to fibers and/or yarn having a weight (g/m²) of less than about 1 g/m², or less than about 2 g/m², or less than about 3 g/m², or less than about 4 g/m², or less than about 5 g/m², or less than about 6 g/m², or less than about 7 g/m², or less than about 8 g/m², or less than about 9 g/m², or less than about 10 g/m², or less than about 20 g/m², or less than about 30 g/m², or less than about 40 g/m², or less than about 50 g/m², or less than about 60 g/m², or less than about 70 g/m², or less than about 80 g/m², or less than about 90 g/m², or less than about 100 g/m², or less than about 200 g/m², or less than about 300 g/m², or less than about 400 g/m², or less than about 500 g/m².

In some embodiments, SFS may be applied to fibers and/or yarn having a weight (g/m²) of at greater than about 1 g/m², or greater than about 2 g/m², or greater than about 3 g/m², or greater than about 4 g/m², or greater than about 5 g/m², or greater than about 6 g/m², or greater than about 7 g/m², or greater than about 8 g/m², or greater than about 9 g/m², or greater than about 10 g/m², or greater than about 20 g/m², or greater than about 30 g/m², or greater than about 40 g/m², or greater than about 50 g/m², or greater than about 60 g/m², or greater than about 70 g/m², or greater than about 80 g/m², or greater than about 90 g/m², or greater than about 100 g/m², or greater than about 200 g/m², or greater than about 300 g/m², or greater than about 400 g/m², or greater than about 500 g/m².

In some embodiments, SFS may be applied to fabric having a thickness of less than about 100 nm, or less than about 200 nm, or less than about 300 nm, or less than about 400 nm, or less than about 500 nm, or less than about 600 nm, or less than about 700 nm, or less than about 800 nm, or less than about 900 nm, or less than about 1000 nm, or less than about 2 μm, or less than about 5 μm, or less than about 10 μm, or less than about 20 μm, or less than about 30 μm, or less than about 40 μm, or less than about 50 μm, or less than about 60 μm, or less than about 70 μm, or less than about 80 μm, or less than about 90 μm, or less than about 100 μm, or less than about 200 μm, or less than about 300 μm, or less than about 400 μm, or less than about 500 μm, or less than about 600 μm, or less than about 700 μm, or less than about 800 μm, or less than about 900 μm, or less than about 1000 μm, or less than about 2 mm, or less than about 3 mm, or less than about 4 mm, or less than about 5 mm, or less than about 6 mm, or less than about 7 mm, or less than about 8 mm, or less than about 9 mm, or less than about 10 mm.

In some embodiments, SFS may be applied to fabric having a thickness of greater than about 100 nm, or greater than about 200 nm, or greater than about 300 nm, or greater than about 400 nm, or greater than about 500 nm, or greater than about 600 nm, or greater than about 700 nm, or greater than about 800 nm, or greater than about 900 nm, or greater than about 1000 nm, or greater than about 2 μm, or greater than about 5 μm, or greater than about 10 μm, or greater than about 20 μm, or greater than about 30 μm, or greater than about 40 μm, or greater than about 50 μm, or greater than about 60 μm, or greater than about 70 μm, or greater than about 80 μm, or greater than about 90 μm, or greater than about 100 μm, or greater than about 200 μm, or greater than about 300 μm, or greater than about 400 μm, or greater than about 500 μm, or greater than about 600 μm, or greater than about 700 μm, or greater than about 800 μm, or greater than about 900 μm, or greater than about 1000 μm, or greater than about 2 mm, or greater than about 3 mm, or greater than about 4 mm, or greater than about 5 mm, or greater than about 6 mm, or greater than about 7 mm, or greater than about 8 mm, or greater than about 9 mm, or greater than about 10 mm.

In some embodiments, SFS may be applied to fabric having a width of less than about 100 nm, or less than about 200 nm, or less than about 300 nm, or less than about 400 nm, or less than about 500 nm, or less than about 600 nm, or less than about 700 nm, or less than about 800 nm, or less than about 900 nm, or less than about 1000 nm, or less than about 2 μm, or less than about 5 μm, or less than about 10 μm, or less than about 20 μm, or less than about 30 μm, or less than about 40 μm, or less than about 50 μm, or less than about 60 μm, or less than about 70 μm, or less than about 80 μm, or less than about 90 μm, or less than about 100 μm, or less than about 200 μm, or less than about 300 μm, or less than about 400 μm, or less than about 500 μm, or less than about 600 μm, or less than about 700 μm, or less than about 800 μm, or less than about 900 μm, or less than about 1000 μm, or less than about 2 mm, or less than about 3 mm, or less than about 4 mm, or less than about 5 mm, or less than about 6 mm, or less than about 7 mm, or less than about 8 mm, or less than about 9 mm, or less than about 10 mm, or less than about 20 mm, or less than about 30 mm, or less than about 40 mm, or less than about 50 mm, or less than about 60 mm, or less than about 70 mm, or less than about 80 mm, or less than about 90 mm, or less than about 100 mm, or less than about 200 mm, or less than about 300 mm, or less than about 400 mm, or less than about 500 mm, or less than about 600 mm, or less than about 700 mm, or less than about 800 mm, or less than about 900 mm, or less than about 1000 mm, or less than about 2 m, or less than about 3 m, or less than about 4 m, or less than about 5 m.

In some embodiments, SFS may be applied to fabric having a width of greater than about 100 nm, or greater than about 200 nm, or greater than about 300 nm, or greater than about 400 nm, or greater than about 500 nm, or greater than about 600 nm, or greater than about 700 nm, or greater than about 800 nm, or greater than about 900 nm, or greater than about 1000 nm, or greater than about 2 μm, or greater than about 5 μm, or greater than about 10 μm, or greater than about 20 μm, or greater than about 30 μm, or greater than about 40 μm, or greater than about 50 μm, or greater than about 60 μm, or greater than about 70 μm, or greater than about 80 μm, or greater than about 90 μm, or greater than about 100 μm, or greater than about 200 μm, or greater than about 300 μm, or greater than about 400 μm, or greater than about 500 μm, or greater than about 600 μm, or greater than about 700 μm, or greater than about 800 μm, or greater than about 900 μm, or greater than about 1000 μm, or greater than about 2 mm, or greater than about 3 mm, or greater than about 4 mm, or greater than about 5 mm, or greater than about 6 mm, or greater than about 7 mm, or greater than about 8 mm, or greater than about 9 mm, or greater than about 10 mm, or greater than about 20 mm, or greater than about 30 mm, or greater than about 40 mm, or greater than about 50 mm, or greater than about 60 mm, or greater than about 70 mm, or greater than about 80 mm, or greater than about 90 mm, or greater than about 100 mm, or greater than about 200 mm, or greater than about 300 mm, or greater than about 400 mm, or greater than about 500 mm, or greater than about 600 mm, or greater than about 700 mm, or greater than about 800 mm, or greater than about 900 mm, or greater than about 1000 mm, or greater than about 2 m, or greater than about 3 m, or greater than about 4 m, or greater than about 5 m.

In some embodiments, SFS may be applied to fabric having a length of less than about 100 nm, or less than about 200 nm, or less than about 300 nm, or less than about 400 nm, or less than about 500 nm, or less than about 600 nm, or less than about 700 nm, or less than about 800 nm, or less than about 900 nm, or less than about 1000 nm, or less than about 2 μm, or less than about 5 μm, or less than about 10 μm, or less than about 20 μm, or less than about 30 μm, or less than about 40 μm, or less than about 50 μm, or less than about 60 μm, or less than about 70 μm, or less than about 80 μm, or less than about 90 μm, or less than about 100 μm, or less than about 200 μm, or less than about 300 μm, or less than about 400 μm, or less than about 500 μm, or less than about 600 μm, or less than about 700 μm, or less than about 800 μm, or less than about 900 μm, or less than about 1000 μm, or less than about 2 mm, or less than about 3 mm, or less than about 4 mm, or less than about 5 mm, or less than about, or less than about 6 mm, or less than about 7 mm, or less than about 8 mm, or less than about 9 mm, or less than about 10 mm, or less than about 20 mm, or less than about 30 mm, or less than about 40 mm, or less than about 50 mm, or less than about 60 mm, or less than about 70 mm, or less than about 80 mm, or less than about 90 mm, or less than about 100 mm, or less than about 200 mm, or less than about 300 mm, or less than about 400 mm, or less than about 500 mm, or less than about 600 mm, or less than about 700 mm, or less than about 800 mm, or less than about 900 mm, or less than about 1000 mm.

In some embodiments, SFS may be applied to fabric having a length of greater than about 100 nm, or greater than about 200 nm, or greater than about 300 nm, or greater than about 400 nm, or greater than about 500 nm, or greater than about 600 nm, or greater than about 700 nm, or greater than about 800 nm, or greater than about 900 nm, or greater than about 1000 nm, or greater than about 2 μm, or greater than about 5 μm, or greater than about 10 μm, or greater than about 20 μm, or greater than about 30 μm, or greater than about 40 μm, or greater than about 50 μm, or greater than about 60 μm, or greater than about 70 μm, or greater than about 80 μm, or greater than about 90 μm, or greater than about 100 μm, or greater than about 200 μm, or greater than about 300 μm, or greater than about 400 μm, or greater than about 500 μm, or greater than about 600 μm, or greater than about 700 μm, or greater than about 800 μm, or greater than about 900 μm, or greater than about 1000 μm, or greater than about 2 mm, or greater than about 3 mm, or greater than about 4 mm, or greater than about 5 mm, or greater than about 6 mm, or greater than about 7 mm, or greater than about 8 mm, or greater than about 9 mm, or greater than about 10 mm, or greater than about 20 mm, or greater than about 30 mm, or greater than about 40 mm, or greater than about 50 mm, or greater than about 60 mm, or greater than about 70 mm, or greater than about 80 mm, or greater than about 90 mm, or greater than about 100 mm, or greater than about 200 mm, or greater than about 300 mm, or greater than about 400 mm, or greater than about 500 mm, or greater than about 600 mm, or greater than about 700 mm, or greater than about 800 mm, or greater than about 900 mm, or greater than about 1000 mm.

In some embodiments, SFS may be applied to fabric having a stretch percentage of less than about 1%, or less than about 2%, or less than about 3%, or less than about 4%, or less than about 5%, or less than about 6%, or less than about 7%, or less than about 8%, or less than about 9%, or less than about 10%, or less than about 20%, or less than about 30%, or less than about 40%, or less than about 50%, or less than about 60%, or less than about 70%, or less than about 80%, or less than about 90%, or less than about 100, or less than about 110%, or less than about 120%, or less than about 130%, or less than about 140%, or less than about 150%, or less than about 160%, or less than about 170%, or less than about 180%, or less than about 190%, or less than about 200%. Stretch percentage may be determined for a fabric having an unstretched width and stretching the fabric to a stretched width, then subtracting the unstretched width from the stretched width to yield the net stretched width, then dividing the net stretched width and multiplying the

$$( Stretch Percentage = \frac{( Stretched Width - Unstretched Width )}{Unstretched Width} * 100 ) .$$

In some embodiments, SFS may be applied to fabric having a stretch percentage of greater than about 1%, or greater than about 2%, or greater than about 3%, or greater than about 4%, or greater than about 5%, or greater than about 6%, or greater than about 7%, or greater than about 8%, or greater than about 9%, or greater than about 10%, or greater than about 20%, or greater than about 30%, or greater than about 40%, or greater than about 50%, or greater than about 60%, or greater than about 70%, or greater than about 80%, or greater than about 90%, or greater than about 100, or greater than about 110%, or greater than about 120%, or greater than about 130%, or greater than about 140%, or greater than about 150%, or greater than about 160%, or greater than about 170%, or greater than about 180%, or greater than about 190%, or greater than about 200%.

In some embodiments, SFS may be applied to fabric having a tensile energy (N/cm²) of less than about 1 cN/cm², or less than about 2 cN/cm², or less than about 3 cN/cm², or less than about 4 cN/cm², or less than about 5 cN/cm², or less than about 5 cN/cm², or less than about 6 cN/cm², or less than about 7 cN/cm², or less than about 8 cN/cm², or less than about 9 cN/cm², or less than about 10 cN/cm², or less than about 20 cN/cm², or less than about 30 cN/cm², or less than about 40 cN/cm², or less than about 50 cN/cm², or less than about 60 cN/cm², or less than about 70 cN/cm², or less than about 80 cN/cm², or less than about 90 cN/cm², or less than about 100 cN/cm², or less than about 2 N/cm², or less than about 3 N/cm², or less than about 4 N/cm², or less than about 5 N/cm², or less than about 6 N/cm², or less than about 7 N/cm², or less than about 8 N/cm², or less than about 9 N/cm², or less than about 10 N/cm², or less than about 20 N/cm², or less than about 30 N/cm², or less than about 40 N/cm², or less than about 50 N/cm², or less than about 60 N/cm², or less than about 70 N/cm², or less than about 80 N/cm², or less than about 90 N/cm², or less than about 100 N/cm², or less than about 150 N/cm², or less than about 200 N/cm².

In some embodiments, SFS may be applied to fabric having a tensile energy (N/cm²) of greater than about 1 cN/cm², or greater than about 2 cN/cm², or greater than about 3 cN/cm², or greater than about 4 cN/cm², or greater than about 5 cN/cm², or greater than about 5 cN/cm², or greater than about 6 cN/cm², or greater than about 7 cN/cm², or greater than about 8 cN/cm², or greater than about 9 cN/cm², or greater than about 10 cN/cm², or greater than about 20 cN/cm², or greater than about 30 cN/cm², or greater than about 40 cN/cm², or greater than about 50 cN/cm², or greater than about 60 cN/cm², or greater than about 70 cN/cm², or greater than about 80 cN/cm², or greater than about 90 cN/cm², or greater than about 100 cN/cm², or greater than about 2 N/cm², or greater than about 3 N/cm², or greater than about 4 N/cm², or greater than about 5 N/cm², or greater than about 6 N/cm², or greater than about 7 N/cm², or greater than about 8 N/cm², or greater than about 9 N/cm², or greater than about 10 N/cm², or greater than about 20 N/cm², or greater than about 30 N/cm², or greater than about 40 N/cm², or greater than about 50 N/cm², or greater than about 60 N/cm², or greater than about 70 N/cm², or greater than about 80 N/cm², or greater than about 90 N/cm², or greater than about 100 N/cm², or greater than about 150 N/cm², or greater than about 200 N/cm².

In some embodiments, SFS may be applied to fabric having a shear rigidity (N/cm-degree) of less than about 1 cN/cm-degree, or less than about 2 cN/cm-degree, or less than about 3 cN/cm-degree, or less than about 4 cN/cm-degree, or less than about 5 cN/cm-degree, or less than about 5 cN/cm-degree, or less than about 6 cN/cm-degree, or less than about 7 cN/cm-degree, or less than about 8 cN/cm-degree, or less than about 9 cN/cm-degree, or less than about 10 cN/cm-degree, or less than about 20 cN/cm-degree, or less than about 30 cN/cm-degree, or less than about 40 cN/cm-degree, or less than about 50 cN/cm-degree, or less than about 60 cN/cm-degree, or less than about 70 cN/cm-degree, or less than about 80 cN/cm-degree, or less than about 90 cN/cm-degree, or less than about 100 cN/cm-degree, or less than about 2 N/cm-degree, or less than about 3 N/cm-degree, or less than about 4 N/cm-degree, or less than about 5 N/cm-degree, or less than about 6 N/cm-degree, or less than about 7 N/cm-degree, or less than about 8 N/cm-degree, or less than about 9 N/cm-degree, or less than about 10 N/cm-degree, or less than about 20 N/cm-degree, or less than about 30 N/cm-degree, or less than about 40 N/cm-degree, or less than about 50 N/cm-degree, or less than about 60 N/cm-degree, or less than about 70 N/cm-degree, or less than about 80 N/cm-degree, or less than about 90 N/cm-degree, or less than about 100 N/cm-degree, or less than about 150 N/cm-degree, or less than about 200 N/cm-degree.

In some embodiments, SFS may be applied to fabric having a shear rigidity (N/cm-degree) of greater than about 1 cN/cm-degree, or greater than about 2 cN/cm-degree, or greater than about 3 cN/cm-degree, or greater than about 4 cN/cm-degree, or greater than about 5 cN/cm-degree, or greater than about 5 cN/cm-degree, or greater than about 6 cN/cm-degree, or greater than about 7 cN/cm-degree, or greater than about 8 cN/cm-degree, or greater than about 9 cN/cm-degree, or greater than about 10 cN/cm-degree, or greater than about 20 cN/cm-degree, or greater than about 30 cN/cm-degree, or greater than about 40 cN/cm-degree, or greater than about 50 cN/cm-degree, or greater than about 60 cN/cm-degree, or greater than about 70 cN/cm-degree, or greater than about 80 cN/cm-degree, or greater than about 90 cN/cm-degree, or greater than about 100 cN/cm-degree, or greater than about 2 N/cm-degree, or greater than about 3 N/cm-degree, or greater than about 4 N/cm-degree, or greater than about 5 N/cm-degree, or greater than about 6 N/cm-degree, or greater than about 7 N/cm-degree, or greater than about 8 N/cm-degree, or greater than about 9 N/cm-degree, or greater than about 10 N/cm-degree, or greater than about 20 N/cm-degree, or greater than about 30 N/cm-degree, or greater than about 40 N/cm-degree, or greater than about 50 N/cm-degree, or greater than about 60 N/cm-degree, or greater than about 70 N/cm-degree, or greater than about 80 N/cm-degree, or greater than about 90 N/cm-degree, or greater than about 100 N/cm-degree, or greater than about 150 N/cm-degree, or greater than about 200 N/cm-degree.

In some embodiments, SFS may be applied to fabric having a bending rigidity (N·cm²/cm) of less than about 1 cN·cm²/cm, or less than about 2 cN·cm²/cm, or less than about 3 cN·cm²/cm, or less than about 4 cN·cm²/cm, or less than about 5 cN·cm²/cm, or less than about 5 cN·cm²/cm, or less than about 6 cN·cm²/cm, or less than about 7 cN·cm²/cm, or less than about 8 cN·cm²/cm, or less than about 9 cN·cm²/cm, or less than about 10 cN·cm²/cm, or less than about 20 cN·cm²/cm, or less than about 30 cN·cm²/cm, or less than about 40 cN·cm²/cm, or less than about 50 cN·cm²/cm, or less than about 60 cN·cm²/cm, or less than about 70 cN·cm²/cm, or less than about 80 cN·cm²/cm, or less than about 90 cN·cm²/cm, or less than about 100 cN·cm²/cm, or less than about 2 N·cm²/cm, or less than about 3 N·cm²/cm, or less than about 4 N·cm²/cm, or less than about 5 N·cm²/cm, or less than about 6 N·cm²/cm, or less than about 7 N·cm²/cm, or less than about 8 N·cm²/cm, or less than about 9 N·cm²/cm, or less than about 10 N·cm²/cm, or less than about 20 N·cm²/cm, or less than about 30 N·cm²/cm, or less than about 40 N·cm²/cm, or less than about 50 N·cm²/cm, or less than about 60 N·cm²/cm, or less than about 70 N·cm²/cm, or less than about 80 N·cm²/cm, or less than about 90 N·cm²/cm, or less than about 100 N·cm²/cm, or less than about 150 N·cm²/cm, or less than about 200 N·cm²/cm. In some embodiments, SFS may be applied to fabric having a bending rigidity (N·cm²/cm) of greater than about 1 cN·cm²/cm, or greater than about 2 cN·cm²/cm, or greater than about 3 cN·cm²/cm, or greater than about 4 cN·cm²/cm, or greater than about 5 cN·cm²/cm, or greater than about 5 cN·cm²/cm, or greater than about 6 cN·cm²/cm, or greater than about 7 cN·cm²/cm, or greater than about 8 cN·cm²/cm, or greater than about 9 cN·cm²/cm, or greater than about 10 cN·cm²/cm, or greater than about 20 cN·cm²/cm, or greater than about 30 cN·cm²/cm, or greater than about 40 cN·cm²/cm, or greater than about 50 cN·cm²/cm, or greater than about 60 cN·cm²/cm, or greater than about 70 cN·cm²/cm, or greater than about 80 cN·cm²/cm, or greater than about 90 cN·cm²/cm, or greater than about 100 cN·cm²/cm, or greater than about 2 N·cm²/cm, or greater than about 3 N·cm²/cm, or greater than about 4 N·cm²/cm, or greater than about 5 N·cm²/cm, or greater than about 6 N·cm²/cm, or greater than about 7 N·cm²/cm, or greater than about 8 N·cm²/cm, or greater than about 9 N·cm²/cm, or greater than about 10 N·cm²/cm, or greater than about 20 N·cm²/cm, or greater than about 30 N·cm²/cm, or greater than about 40 N·cm²/cm, or greater than about 50 N·cm²/cm, or greater than about 60 N·cm²/cm, or greater than about 70 N·cm²/cm, or greater than about 80 N·cm²/cm, or greater than about 90 N·cm²/cm, or greater than about 100 N·cm²/cm, or greater than about 150 N·cm²/cm, or greater than about 200 N·cm²/cm.

In some embodiments, SFS may be applied to fabric having a compression energy (N·cm/cm²) of less than about 1 cN·cm/cm², or less than about 2 cN·cm/cm², or less than about 3 cN·cm/cm², or less than about 4 cN·cm/cm², or less than about 5 c N·cm/cm², or less than about 5 cN·cm/cm², or less than about 6 cN·cm/cm², or less than about 7 cN·cm/cm², or less than about 8 cN·cm/cm², or less than about 9 cN·cm/cm², or less than about 10 cN·cm/cm², or less than about 20 cN·cm/cm², or less than about 30 cN·cm/cm², or less than about 40 cN·cm/cm², or less than about 50 cN·cm/cm², or less than about 60 cN·cm/cm², or less than about 70 cN·cm/cm², or less than about 80 cN·cm/cm², or less than about 90 cN·cm/cm², or less than about 100 cN·cm/cm², or less than about 2 N·cm/cm², or less than about 3 N·cm/cm², or less than about 4 N·cm/cm², or less than about 5 N·cm/cm², or less than about 6 N·cm/cm², or less than about 7 N·cm/cm², or less than about 8 N·cm/cm², or less than about 9 N·cm/cm², or less than about 10 N·cm/cm², or less than about 20 N·cm/cm², or less than about 30 N·cm/cm², or less than about 40 N·cm/cm², or less than about 50 N·cm/cm², or less than about 60 N·cm/cm², or less than about 70 N·cm/cm², or less than about 80 N·cm/cm², or less than about 90 N·cm/cm², or less than about 100 N·cm/cm², or less than about 150 N·cm/cm², or less than about 200 N·cm/cm².

In some embodiments, SFS may be applied to fabric having a compression energy (N·cm/cm²) of greater than about 1 cN·cm/cm², or greater than about 2 cN·cm/cm², or greater than about 3 cN·cm/cm², or greater than about 4 cN·cm/cm², or greater than about 5 cN·cm/cm², or greater than about 5 cN·cm/cm², or greater than about 6 cN·cm/cm², or greater than about 7 cN·cm/cm², or greater than about 8 cN·cm/cm², or greater than about 9 cN·cm/cm², or greater than about 10 cN·cm/cm², or greater than about 20 cN·cm/cm², or greater than about 30 cN·cm/cm², or greater than about 40 cN·cm/cm², or greater than about 50 cN·cm/cm², or greater than about 60 cN·cm/cm², or greater than about 70 cN·cm/cm², or greater than about 80 cN·cm/cm², or greater than about 90 cN·cm/cm², or greater than about 100 cN·cm/cm², or greater than about 2 N·cm/cm², or greater than about 3 N·cm/cm², or greater than about 4 N·cm/cm², or greater than about 5 N·cm/cm², or greater than about 6 N·cm/cm², or greater than about 7 N·cm/cm², or greater than about 8 N·cm/cm², or greater than about 9 N·cm/cm², or greater than about 10 N·cm/cm², or greater than about 20 N·cm/cm², or greater than about 30 N·cm/cm², or greater than about 40 N·cm/cm², or greater than about 50 N·cm/cm², or greater than about 60 N·cm/cm², or greater than about 70 N·cm/cm², or greater than about 80 N·cm/cm², or greater than about 90 N·cm/cm², or greater than about 100 N·cm/cm², or greater than about 150 N·cm/cm², or greater than about 200 N·cm/cm².

In some embodiments, SFS may be applied to fabric having a coefficient of friction of less than about 0.04, or less than about 0.05, or less than about 0.06, or less than about 0.07, or less than about 0.08, or less than about 0.09, or less than about 0.10, or less than about 0.10, or less than about 0.15, or less than about 0.20, or less than about 0.25, or less than about 0.30, or less than about 0.35, or less than about 0.40, or less than about 0.45, or less than about 0.50, or less than about 0.55, or less than about 0.60, or less than about 0.65, or less than about 0.70, or less than about 0.75, or less than about 0.80, or less than about 0.85, or less than about 0.90, or less than about 0.95, or less than about 1.00, or less than about 1.05.

In some embodiments, SFS may be applied to fabric having a coefficient of friction of greater than about 0.04, or greater than about 0.05, or greater than about 0.06, or greater than about 0.07, or greater than about 0.08, or greater than about 0.09, or greater than about 0.10, or greater than about 0.10, or greater than about 0.15, or greater than about 0.20, or greater than about 0.25, or greater than about 0.30, or greater than about 0.35, or greater than about 0.40, or greater than about 0.45, or greater than about 0.50, or greater than about 0.55, or greater than about 0.60, or greater than about 0.65, or greater than about 0.70, or greater than about 0.75, or greater than about 0.80, or greater than about 0.85, or greater than about 0.90, or greater than about 0.95, or greater than about 1.00, or greater than about 1.05.

In some embodiments, chemical finishes may be applied to textiles before or after such textiles are coated with SFS. In an embodiment, chemical finishing may be intended as the application of chemical agents and/or SFS to textiles, including fibers, yarn, and fabric, or to garments that are prepared by such fibers, yarn, and fabric to modify the original textile's or garment's properties and achieve properties in the textile or garment that would be otherwise absent. With chemical finishes, textiles treated with such chemical finishes may act as surface treatments and/or the treatments may modify the elemental analysis of treated textile base polymers.

In an embodiment, a type of chemical finishing may include the application of certain silk-fibroin based solutions to textiles. For example, SFS may be applied to a fabric after it is dyed, but there are also scenarios that may require the application of SFS during processing, during dyeing, or after a garment is assembled from a selected textile or fabric, thread, or yarn. In some embodiments, after its application, SFS may be dried with the use of heat. SFS may then be fixed to the surface of the textile in a processing step called curing.

In some embodiments, SFS may be supplied in a concentrated form suspended in water. In some embodiments, SFS may have a concentration by weight (% w/w or % w/v) or by volume (v/v) of less than about 50%, or less than about 45%, or less than about 40%, or less than about 35%, or less than about 30%, or less than about 25%, or less than about 20%, or less than about 15%, or less than about 10%, or less than about 5%, or less than about 4%, or less than about 3%, or less than about 2%, or less than about 1%, or less than about 0.1%, or less than about 0.01%, or less than about 0.001%, or less than about 0.0001%, or less than about 0.00001%. In some embodiments, SFS may have a concentration by weight (% w/w or % w/v) or by volume (v/v) of greater than about 50%, or greater than about 45%, or greater than about 40%, or greater than about 35%, or greater than about 30%, or greater than about 25%, or greater than about 20%, or greater than about 15%, or greater than about 10%, or greater than about 5%, or greater than about 4%, or greater than about 3%, or greater than about 2%, or greater than about 1%, or greater than about 0.1%, or greater than about 0.01%, or greater than about 0.001%, or greater than about 0.0001%, or greater than about 0.00001%.

In some embodiments, the solution concentration and the wet pick of the material determines the amount of silk fibroin solution (SFS), which may include silk-based proteins or fragments thereof, that may be fixed or otherwise adhered to the textile being coated. The wet pick up may be expressed by the following formula:

$$wet pick up ( \% ) = \frac{weight of SFS applied \times 100}{weight of dry textile material} .$$

The total amount of SFS added to the textile material may be expressed by the following formula:

$$SFS added ( \% ) = \frac{weight of dry SFS coated textile material \times 100}{weight of dry textile material before coating} .$$

Regarding methods for applying SFS to textiles more broadly, SFS may be applied to textiles through a pad or roller application on process, a saturation and removal process, and/or a topical application process. Moreover, the methods of silk application (i.e., SFS application or coating) may include bath coating, kiss rolling, spray coating, and/or two-sided rolling. In some embodiments, the coating processes (e.g., bath coating, kiss rolling, spray coating, two-sided rolling, roller application, saturation and removal application, and/or topical application), drying processes, and curing processes may be varied as described herein to modify one or more selected textile (e.g., fabric) properties of the resulting coated textile wherein such properties include, but are not limited to wetting time, absorption rate, spreading speed, accumulative one-way transport, and/or overall moisture management capability. In some embodiments, the aforementioned selected properties may be enhanced by varying one or more of the coating processes, drying processes, and curing processes as described herein.

In an embodiment, the padder application may be used on dry or wet textile. For example, it may be applied on fabric after the dyeing process. The fabric may be fed into a water bath solution and may reach saturation. The fabric to be coated may then pass through a set of rollers that, based on multiple variables, extract the bath solution in excess to the desired wet pick up %. The variables that affect the wet pick up % are the roller pressure and materials, the fabric composition and construction, and the SFS viscosity.

In an embodiment, the padder application on wet textile may be used to reduce the cost of drying the fabric post dyeing. The fabric exiting the pad rollers may maintain a higher weight % than the incoming fabric to maintain a SFS deposit on the fabric; and the SFS solution may need to account for any dilution taking place due to water present on the incoming fabric.

In an embodiment, the saturation and removal application is a low wet pick up method that may, for example, solve some of the issues associated with removing large amounts of water during drying processes. Since fabric may dry in an oven from the outside surface towards the inside, water may move from the inside to the outside resulting in a higher coating concentration on the outside surface. With less water content, migration may be reduced due to a higher viscosity in the solution. However, decreased wet pick up may result in an uneven solution deposit.

In an embodiment, vacuum extraction may be used as a method for low wet pick up. Saturated fabric may be subject to a vacuum that pulls solution out of the fabric and returns it to an application loop. Air jet ejection may be a method for providing low wet pick up. The saturated fabric may be subjected to high pressure steam that removes solution out of the fabric and returns it to an application loop.

In an embodiment, a porous bowl method may be used for low wet pick up. Solid pad rollers may be substituted with rubber coated fiber rollers. Saturated fabric may be subjected to the pressure of the roller since the porosity of the rollers may allow for more solution to be squeezed from the fabric.

In an embodiment, a transfer padding method may be used for low wet pick up. Saturated fabric may be passed through two continuous dry non-woven fabrics and may be pressed at low pressure. The non-woven fabrics may extract excess solution from the fabric being treated.

In an embodiment, topical application may be used as a low wet pick up method of application that deposits the desired amount of SFS to the fabric without removing any excess material. The methods described above may be used for one-sided coating applications, but there are variations that may allow for two-sided coating.

In an embodiment, kiss rolling may be used as a topical method of application that transfers the SFS from a roller (i.e., a kiss roller) to one side of the fabric. The solution viscosity, roller surface finish, speed of the roller, speed of the fabric, contact angle of the fabric on the roller and properties of the fabric are parameters that control the amount of solution deposited on the fabric.

In an embodiment, a variation to the kiss roller technique may be the Triatex MA system that uses two moisture content sensors to determine the solution pick up at the kiss roller and adjust the kiss roller controllable variable to maintain consistent the solution deposit onto the fabric.

In an embodiment, a loop transfer application may be used as a topical method of application that transfers the SFS from a saturated loop fabric to the fabric to be coated between low pressure pad rollers. There is a two rollers version that may allow for minimum contact with the fabric and a three rollers version that allows for greater contact with the fabric.

In an embodiment, an engrave roller application may be used as a topical method of application that may transfer a metered amount of SFS onto the fabric. This may be achieved by engraving a pattern on the surface of the roller with precise depth and design that contains a controlled amount of SFS. A blade may be used to remove any solution that is deposited on the surface of the roller in order to maintain a consistent transfer of solution to the fabric to be coated.

In an embodiment, rotary screen printing may be used as a topical method of application that may deposit SFS onto the fabric by seeping the solution through a roller screen. The solution may be contained in the screen print roller core at a set level while a blade may be used to remove any excess solution from the interior roller wall, providing a clean surface for the next revolution of the screen printer roller.

In an embodiment, magnetic roller coating may be used as a topical method of application that may deposit SFS from a kiss roller onto the fabric to be coated. The kiss roller is semi-submersed in a bath solution while a magnetic field created in the fabric driving roller determines the amount of pressure applied by the kiss roller, controlling the solution pick up rate.

In an embodiment, spraying may be used as a topical method of application that may transfer SFS onto the fabric by nebulizing the solution. The spray pattern may be controlled by the nozzle pattern, size, and the air flow. Spray application may be used for one side application or also two sided application.

In an embodiment, foam application may be used a topical method of application that may transfer SFS onto the fabric. Foam may be made by substituting part of the water in the solution with air therefore reducing the amount of water to be applied to the fabric. Foam application may be used for one-sided application or two-sided application where the same foam may be deposited through a squeeze roller or different foam solutions may be provided through transfer rolls or through a slot applicator.

In an embodiment, the application of SFS may take place after a garment is assembled. In an embodiment, the process may take place in a washing and dyeing machine or in a spray booth. For example, a washing and dyeing machine may be similar in shape to a household front loader washing machine, it allows the process to take place at exhaustion post dyeing or with an independent processing cycle. In an embodiment, a spray booth machine may include a manual or a fully automated process. For example, a garment may be held by a mannequin while an operator or an anthropomorphic robot may spray the solution onto the fabric.

In an embodiment, SFS may be a water based solution that, after its application to the textile, may require thermal vaporization to infuse the SFS onto the textile. Thermal vaporization may be applied by heat transfer through radiation with equipment such as infrared or radio frequency dryer.

In an embodiment, thermal vaporization may be applied by convection through heated air circulating in an oven to the required temperature, while the fabric is clamped and is transported by a conveyor. This allows full control on fabric width dimension.

In an embodiment, thermal vaporization may be applied by conduction through contacting the textile with heated cylinder or calendar cylinder. Since the fabric is not clamp there is minimal control on fabric width.

In an embodiment, curing of the SFS on the textile may be completed with the same equipment used for the thermal vaporization in a continuous cycle or in a separate cycle.

In an embodiment, curing time temperature may be dependent the textile polymer content and the binding method of preference for the SFS with the specific polymer. The curing process may not start until the thermal vaporization is completed.

In some embodiments, sensor may be used to monitor SFS deposition on the textile and the drying and curing steps.

In some embodiments, for monitoring the deposition of SFS, a contactless sensor, like the one supplied by Pleva model AF120 based on microwave absorption of water, may be used. Measurement of the material moisture may be based on microwave absorption by water. A semiconductor oscillator transmits microwave energy through the web. The non-absorbed part of the energy may be received on the opposite side by a microwave receiver. The amount of absorption is a measurement of the absolute moisture content. The microwave sensor is capable of detecting and measuring water content from a minimum of 0 up to 2000 gH₂O/m².

In some embodiments, for wide fabric processing multiple sensor may be paired side by side, delivering the data analysis to a centralized control system loop capable to add more solution in the area of the fabric that is low.

In some embodiments, another sensor may be used that is based on microwave technology, such as Aqualot by Mahlo. The sensor may evaluate the shift in the resonant frequency of the two standing waves with respect to each other rather than the attenuation of the microwaves by the quantity of water molecules in the measuring gap.

In some embodiments, another contactless sensor for SFS may be the IR-3000 by MoistTech based on near infrared sensing technology. The sensor measures the amount of near infrared energy reflected at a given wavelength that is inversely proportional to the quantity of absorbing molecules in the fabric.

In some embodiments, the residual moister at the end of the curing process may be measured to further confirm the drying and curing process. In addition to the above sensor, a contact sensor such as the Textometer RMS by Mahlo may be used for measuring moister through conductivity.

In some embodiments, monitoring the end of the drying process phase may be achieved by measuring the fabric temperature with a contactless temperature sensor. When wet product enters the dryer, it first heats up to the cooling limit temperature. In some embodiments, when the water content drops to residual moisture levels, the product temperature may begin to rise again. The closer the product temperature approaches the circulation air temperature in the dryer, the slower the temperature continues to rise. In some embodiments, at a certain temperature threshold (called the fixing temperature) the temperature necessary for processing, fixing, or condensing is reached.

In some embodiments, to determine the dwell time for a desired product temperature, the surface temperature of the product may be measured without contact at several locations in the dryer using high-temperature resistant infrared pyrometers. Mahlo Permaset VMT is an infrarem Pyrometer that may be assembled in multiple units to monitors temperature through the dryer. Setex is another manufacturer offering fabric temperature sensors for use in dryers and oven like the models WTM V11, V21, and V41.

In some embodiments, SFS may be applied to a textile during exhaust dyeing. In some embodiments, the process may involve loading fabric into a bath, originally known as a batch, and allowing it to come into equilibrium with the solution. Exhaust dyeing may be the ability of the silk fibroin molecules to move from the solution onto the fibers or thread of a textile (substantivity). The substantivity of the silk fibroin may be influenced by temperature or additives, such as salt.

In some embodiments, an exhaust dyeing process may take anywhere from a few minutes to a few hours. When the fabric has been absorbed, or fixed, as much silk fibroin as it can, the bath may be emptied and the fabric may be rinsed to remove any excess solution.

In some embodiments, an important parameter in exhaust dyeing may be what is known as the specific liquor ratio. This describes the ratio of the mass of the fabric to the volume of the SFS bath and determines the amount of silk fibroin deposited on a textile.

In some embodiments, SFS can be applied to a textile during jet dyeing processes. A jet dyeing machine may formed by closed tubular system where the fabric is placed. For transporting the fabric through the tube, a jet of dye liquor is supplied through a venturi. The jet may create turbulence. This may help in SFS penetration along with preventing the fabric from touching the walls of the tube. For example, as the fabric is often exposed to comparatively higher concentrations of liquor within the transport tube, a small SFS bath is needed in the bottom of the vessel. This arrangement may be enough for the smooth movement from rear to front of the vessel.

In some embodiments, SFS may be applied during Paddle dyeing. Paddle dyeing machines may be generally used to many forms of textiles but the method best suits to garments. Heat may be generated through steam injection directly into the coating bath. In an embodiment, a paddle dyeing machine operates through a paddle that circulates both the bath and garments in a perforated central island. It is here that the SFS, water, and steam for heat are added. The overhead paddle machine may be described as a vat with a paddle that has blades of full width. The blades may generally dip a few centimeters into the vat. This action may stir the bath and push garments to be died down, thus keeping them submerged in the dye liquor. In some embodiments, the processing methods set forth herein may be used to apply SFS to textiles with one or more of the following parameters including, but not limited to, fabric speed, solution viscosity, solution added to fabric, fabric range width, drying temperature, drying time, curing time, fabric tension, padder pressure, padder roller shore hardness, stenter temperature, and common drying and curing temperatures. In an embodiment, the processing method parameters may also include a condensation temperature, which may vary depending upon the chemical recipe used to apply the SFS to the textiles.

In an embodiment, the fabric speed for the processes of the disclosure may be less than about 0.1 m/min, or less than about 0.2 m/min, or less than about 0.3 m/min, or less than about 0.4 m/min, or less than about 0.5 m/min, or less than about 0.6 m/min, or less than about 0.7 m/min, or less than about 0.8 m/min, or less than about 0.9 m/min, or less than about 1 m/min, or less than about 2 m/min, or less than about 3 m/min, or less than about 4 m/min, or less than about 5 m/min, or less than about 6 m/min, or less than about 7 m/min, or less than about 8 m/min, or less than about 9 m/min, or less than about 10 m/min, or less than about 20 m/min, or less than about 30 m/min, or less than about 40 m/min, or less than about 50 m/min, or less than about 60 m/min.

In an embodiment, the fabric speed for the processes of the disclosure may be greater than about 0.1 m/min, or greater than about 0.2 m/min, or greater than about 0.3 m/min, or greater than about 0.4 m/min, or greater than about 0.5 m/min, or greater than about 0.6 m/min, or greater than about 0.7 m/min, or greater than about 0.8 m/min, or greater than about 0.9 m/min, or greater than about 1 m/min, or greater than about 2 m/min, or greater than about 3 m/min, or greater than about 4 m/min, or greater than about 5 m/min, or greater than about 6 m/min, or greater than about 7 m/min, or greater than about 8 m/min, or greater than about 9 m/min, or greater than about 10 m/min, or greater than about 20 m/min, or greater than about 30 m/min, or greater than about 40 m/min, or greater than about 50 m/min, or greater than about 60 m/min.

In an embodiment, the solution viscosity for the processes of the disclosure may be less than about 1000 mPas, or less than about 1500 mPas, or less than about 2000 mPas, or less than about 2500, or less than about 3000 mPas, or less than about 4000 mPas, or less than about 4500 mPas, or less than about 5000 mPas, or less than about 5500 mPas, or less than about 6000 mPas, or less than about 6500 mPas, or less than about 7000 mPas, or less than about 7500 mPas, or less than about 8000 mPas, or less than about 8500 mPas, or less than about 9000 mPas, or less than about 9500 mPas, or less than about 10000 mPas, or less than about 10500 mPas, or less than about 11000 mPas, or less than about 11500 mPas, or less than about 12000 mPas. In an embodiment, the solution viscosity for the processes of the disclosure may be greater than about 1000 mPas, or greater than about 1500 mPas, or greater than about 2000 mPas, or greater than about 2500, or greater than about 3000 mPas, or greater than about 4000 mPas, or greater than about 4500 mPas, or greater than about 5000 mPas, or greater than about 5500 mPas, or greater than about 6000 mPas, or greater than about 6500 mPas, or greater than about 7000 mPas, or greater than about 7500 mPas, or greater than about 8000 mPas, or greater than about 8500 mPas, or greater than about 9000 mPas, or greater than about 9500 mPas, or greater than about 10000 mPas, or greater than about 10500 mPas, or greater than about 11000 mPas, or greater than about 11500 mPas, or greater than about 12000 mPas.

In an embodiment, the solution may be added to a textile (e.g., fabric) for the processes of the disclosure in less than about 0.01 g/m², or less than about 0.02 g/m², or less than about 0.03 g/m², or less than about 0.04 g/m², or less than about 0.05 g/m², or less than about 0.06 g/m², or less than about 0.07 g/m², or less than about 0.08 g/m², or less than about 0.09 g/m², or less than about 0.10 g/m², or less than about 0.2 g/m², or less than about 0.3 g/m², or less than about 0.4 g/m², or less than about 0.5 g/m², or less than about 0.6 g/m², or less than about 0.7 g/m², or less than about 0.8 g/m², or less than about 0.9 g/m², or less than about 1 g/m², or less than about 2 g/m², or less than about 3 g/m², or less than about 4 g/m², or less than about 5 g/m², or less than about 6 g/m², or less than about 7 g/m², or less than about 8 g/m², or less than about 9 g/m², or less than about 10 g/m², or less than about 20 g/m², or less than about 30 g/m², or less than about 40 g/m², or less than about 50 g/m², or less than about 60 g/m², or less than about 70 g/m², or less than about 80 g/m², or less than about 90 g/m², or less than about 100 g/m².

In an embodiment, the solution may be added to a textile (e.g., fabric) for the processes of the disclosure in greater than about 0.01 g/m², or greater than about 0.02 g/m², or greater than about 0.03 g/m², or greater than about 0.04 g/m², or greater than about 0.05 g/m², or greater than about 0.06 g/m², or greater than about 0.07 g/m², or greater than about 0.08 g/m², or greater than about 0.09 g/m², or greater than about 0.10 g/m², or greater than about 0.2 g/m², or greater than about 0.3 g/m², or greater than about 0.4 g/m², or greater than about 0.5 g/m², or greater than about 0.6 g/m², or greater than about 0.7 g/m², or greater than about 0.8 g/m², or greater than about 0.9 g/m², or greater than about 1 g/m², or greater than about 2 g/m², or greater than about 3 g/m², or greater than about 4 g/m², or greater than about 5 g/m², or greater than about 6 g/m², or greater than about 7 g/m², or greater than about 8 g/m², or greater than about 9 g/m², or greater than about 10 g/m², or greater than about 20 g/m², or greater than about 30 g/m², or greater than about 40 g/m², or greater than about 50 g/m², or greater than about 60 g/m², or greater than about 70 g/m², or greater than about 80 g/m², or greater than about 90 g/m², or greater than about 100 g/m².

In an embodiment, the fabric range width for the processes of the disclosure may be less than about 1 mm, or less than about 2 mm, or less than about 3 mm, or less than about 4 mm, or less than about 5 mm, or less than about 6 mm, or less than about 7 mm, or less than about 8 mm, or less than about 9, or less than about 10 mm, or less than about 20 mm, or less than about 30 mm, or less than about 40 mm, or less than about 50 mm, or less than about 60 mm, or less than about 70 mm, or less than about 80 mm, or less than about 90 mm, or less than about 100 mm, or less than about 200, or less than about 300 mm, or less than about 400 mm, or less than about 500 mm, or less than about 600 mm, or less than about 700 mm, or less than about 800 mm, or less than about 900 mm, or less than about 1000 mm, or less than about 2000 mm, or less than about 2000 mm, or less than about 3000 mm, or less than about 4000 mm, or less than about 5000 mm.

In an embodiment, the fabric range width for the processes of the disclosure may be greater than about 1 mm, or greater than about 2 mm, or greater than about 3 mm, or greater than about 4 mm, or greater than about 5 mm, or greater than about 6 mm, or greater than about 7 mm, or greater than about 8 mm, or greater than about 9, or greater than about 10 mm, or greater than about 20 mm, or greater than about 30 mm, or greater than about 40 mm, or greater than about 50 mm, or greater than about 60 mm, or greater than about 70 mm, or greater than about 80 mm, or greater than about 90 mm, or greater than about 100 mm, or greater than about 200, or greater than about 300 mm, or greater than about 400 mm, or greater than about 500 mm, or greater than about 600 mm, or greater than about 700 mm, or greater than about 800 mm, or greater than about 900 mm, or greater than about 1000 mm, or greater than about 2000 mm, or greater than about 2000 mm, or greater than about 3000 mm, or greater than about 4000 mm, or greater than about 5000 mm.

In an embodiment, the drying and/or curing temperature for the processes of the disclosure may be less than about 70° C., or less than about 75° C., or less than about 80° C., or less than about 85° C., or less than about 90° C., or less than about 95° C., or less than about 100° C., or less than about 110° C., or less than about 120° C., or less than about 130° C., or less than about 140° C., or less than about 150° C., or less than about 160° C., or less than about 170° C., or less than about 180° C., or less than about 190° C., or less than about 200° C., or less than about 210° C., or less than about 220° C., or less than about 230° C.

In an embodiment, the drying and/or curing temperature for the processes of the disclosure may be greater than about 70° C., or greater than about 75° C., or greater than about 80° C., or greater than about 85° C., or greater than about 90° C., or greater than about 95° C., or greater than about 100° C., or greater than about 110° C., or greater than about 120° C., or greater than about 130° C., or greater than about 140° C., or greater than about 150° C., or greater than about 160° C., or greater than about 170° C., or greater than about 180° C., or greater than about 190° C., or greater than about 200° C., or greater than about 210° C., or greater than about 220° C., or greater than about 230° C.

In an embodiment, the drying time for the processes of the disclosure may be less than about 10 seconds, or less than about 20 seconds, or less than about 30 seconds, or less than about 40 seconds, or less than about 50 seconds, or less than about 60 seconds, or less than about 2 minutes, or less than about, 3 minutes, or less than about 4 minutes, or less than about 5 minutes, or less than about 6 minutes, or less than about 7 minutes, or less than about 8 minutes, or less than about 9 minutes, or less than about 10 minutes, or less than about 20 minutes, or less than about 30 minutes, or less than about 40 minutes, or less than about 50 minutes, or less than about 60 minutes.

In an embodiment, the drying time for the processes of the disclosure may be greater than about 10 seconds, or greater than about 20 seconds, or greater than about 30 seconds, or greater than about 40 seconds, or greater than about 50 seconds, or greater than about 60 seconds, or greater than about 2 minutes, or greater than about, 3 minutes, or greater than about 4 minutes, or greater than about 5 minutes, or greater than about 6 minutes, or greater than about 7 minutes, or greater than about 8 minutes, or greater than about 9 minutes, or greater than about 10 minutes, or greater than about 20 minutes, or greater than about 30 minutes, or greater than about 40 minutes, or greater than about 50 minutes, or greater than about 60 minutes.

In an embodiment, the curing time for the processes of the disclosure may be less than about 1 second, or less than about 2 seconds, or less than about 3 seconds, or less than about 4 seconds, or less than about 5 seconds, or less than about 6 seconds, or less than about 7 seconds, or less than about 8 seconds, or less than about 9 seconds, or less than about 10 seconds, or less than about 20 seconds, or less than about 30 seconds, or less than about 40 seconds, or less than about 50 seconds, or less than about 60 seconds, or less than about 2 minutes, or less than about 3 minutes, or less than about 4 minutes, or less than about 5 minutes, or less than about 6 minutes, or less than about 7 minutes, or less than about 8 minutes, or less than about 9 minutes, or less than about 10 minutes, or less than about 20 minutes, or less than about 30 minutes, or less than about 40 minutes, or less than about 50 minutes, or less than about 60 minutes.

In an embodiment, the curing time for the processes of the disclosure may be greater than about 1 second, or greater than about 2 seconds, or greater than about 3 seconds, or greater than about 4 seconds, or greater than about 5 seconds, or greater than about 6 seconds, or greater than about 7 seconds, or greater than about 8 seconds, or greater than about 9 seconds, or greater than about 10 seconds, or greater than about 20 seconds, or greater than about 30 seconds, or greater than about 40 seconds, or greater than about 50 seconds, or greater than about 60 seconds, or greater than about 2 minutes, or greater than about 3 minutes, or greater than about 4 minutes, or greater than about 5 minutes, or greater than about 6 minutes, or greater than about 7 minutes, or greater than about 8 minutes, or greater than about 9 minutes, or greater than about 10 minutes, or greater than about 20 minutes, or greater than about 30 minutes, or greater than about 40 minutes, or greater than about 50 minutes, or greater than about 60 minutes.

In an embodiment, the fabric tension for the processes of the disclosure may be less than about 1 N, or less than about 2 N, or less than about 3 N, or less than about 4 N, or less than about 5 N, or less than about 6 N, or less than about 7 N, or less than about 8 N, or less than about 9 N, or less than about 10 N, or less than about 20 N, or less than about 30 N, or less than about 40 N, or less than about 50 N, or less than about 60 N, or less than about 70 N, or less than about 80 N, or less than about 90 N, or less than about 100 N, or less than about 150 N, or less than about 200 N, or less than about 250 N, or less than about 300 N.

In an embodiment, the fabric tension for the processes of the disclosure may be greater than about 1 N, or greater than about 2 N, or greater than about 3 N, or greater than about 4 N, or greater than about 5 N, or greater than about 6 N, or greater than about 7 N, or greater than about 8 N, or greater than about 9 N, or greater than about 10 N, or greater than about 20 N, or greater than about 30 N, or greater than about 40 N, or greater than about 50 N, or greater than about 60 N, or greater than about 70 N, or greater than about 80 N, or greater than about 90 N, or greater than about 100 N, or greater than about 150 N, or greater than about 200 N, or greater than about 250 N, or greater than about 300 N.

In an embodiment, the padder pressure for the processes of the disclosure may be less than about 1 N/mm, or less than about 2 N/mm, or less than about 3 N/mm, or less than about 4 N/mm, or less than about 4 N/mm, or less than about 5 N/mm, or less than about 6 N/mm, or less than about 7 N/mm, or less than about 8 N/mm, or less than about 9 N/mm, or less than about 10 N/mm, or less than about 20 N/mm, or less than about 30 N/mm, or less than about 40 N/mm, or less than about 50 N/mm, or less than about 60 N/mm, or less than about 70 N/mm, or less than about 80 N/mm, or less than about 90 N/mm.

In an embodiment, the padder pressure for the processes of the disclosure may be greater than about 1 N/mm, or greater than about 2 N/mm, or greater than about 3 N/mm, or greater than about 4 N/mm, or greater than about 4 N/mm, or greater than about 5 N/mm, or greater than about 6 N/mm, or greater than about 7 N/mm, or greater than about 8 N/mm, or greater than about 9 N/mm, or greater than about 10 N/mm, or greater than about 20 N/mm, or greater than about 30 N/mm, or greater than about 40 N/mm, or greater than about 50 N/mm, or greater than about 60 N/mm, or greater than about 70 N/mm, or greater than about 80 N/mm, or greater than about 90 N/mm.

In an embodiment, the padder roller shore hardness for the processes of the disclosure may be less than about 70 shore A, or less than about 75 shore A, or less than about 80 shore A, or less than about 85 shore A, or less than about 90 shore A, or less than about 95 shore A, or less than about 100 shore A.

In an embodiment, the padder roller shore hardness for the processes of the disclosure may be greater than about 70 shore A, or greater than about 75 shore A, or greater than about 80 shore A, or greater than about 85 shore A, or greater than about 90 shore A, or greater than about 95 shore A, or greater than about 100 shore A.

In an embodiment, the stenter temperature for the processes of the disclosure may be less than about 70° C., or less than about 75° C., or less than about 80° C., or less than about 85° C., or less than about 90° C., or less than about 95° C., or less than about 100° C., or less than about 110° C., or less than about 120° C., or less than about 130° C., or less than about 140° C., or less than about 150° C., or less than about 160° C., or less than about 170° C., or less than about 180° C., or less than about 190° C., or less than about 200° C., or less than about 210° C., or less than about 220° C., or less than about 230° C.

In an embodiment, the stenter temperature for the processes of the disclosure may be greater than about 70° C., or greater than about 75° C., or greater than about 80° C., or greater than about 85° C., or greater than about 90° C., or greater than about 95° C., or greater than about 100° C., or greater than about 110° C., or greater than about 120° C., or greater than about 130° C., or greater than about 140° C., or greater than about 150° C., or greater than about 160° C., or greater than about 170° C., or greater than about 180° C., or greater than about 190° C., or greater than about 200° C., or greater than about 210° C., or greater than about 220° C., or greater than about 230° C.

In an embodiment, the common drying temperatures for the processes of the disclosure may be less than about 110° C., or less than about 115° C., or less than about 120° C., or less than about 125° C., or less than about 130° C., or less than about 135° C., or less than about 140° C., or less than about 145° C., or less than about 150° C. In an embodiment, the common drying temperatures for the processes of the disclosure may be greater than about 110° C., or greater than about 115° C., or greater than about 120° C., or greater than about 125° C., or greater than about 130° C., or greater than about 135° C., or greater than about 140° C., or greater than about 145° C., or greater than about 150° C.

In some embodiments, a silk fibroin coated material (e.g., fabric) may be heat resistant to a selected temperature where the selected temperature is chosen for drying, curing, and/or heat setting a dye that may be applied to the material (e.g., LYCRA). As used herein, a “heat resistant” may refer to a property of the silk fibroin coating deposited on the material where the silk fibroin coating and/or silk fibroin protein does not exhibit a substantial modification (i.e., “substantially modifying”) in silk fibroin coating performance as compared to a control material having a comparable silk fibroin coating that was not subjected to the selected temperature for drying, curing, wash cycling, and/or heat setting purposes. In some embodiments, the selected temperature is the glass transition temperature (T_{g}) for the material upon which the silk fibroin coating is applied. In some embodiments, the selected temperature is greater than about 65° C., or greater than about 70° C., or greater than about 80° C., or greater than about 90° C., or greater than about 100° C., or greater than about 110° C., or greater than about 120° C., or greater than about 130° C., or greater than about 140° C., or greater than about 150° C., or greater than about 160° C., or greater than about 170° C., or greater than about 180° C., or greater than about 190° C., or greater than about 200° C., or greater than about 210° C., or greater than about 220° C. In some embodiments, the selected temperature is less than about 65° C., or less than about 70° C., or less than about 80° C., or less than about 90° C., or less than about 100° C., or less than about 110° C., or less than about 120° C., or less than about 130° C., or less than about 140° C., or less than about 150° C., or less than about 160° C., or less than about 170° C., or less than about 180° C., or less than about 190° C., or less than about 200° C., or less than about 210° C., or less than about 220° C.

In an embodiment, “substantially modifying” silk fibroin coating performance may be a decrease in a selected property of silk fibroin coating, such as wetting time, absorption rate, spreading speed, accumulative one-way transport, or overall moisture management capability as compared to a control silk fibroin coating that was not subjected to the selected temperature for drying, curing, wash cycling, and/or heat setting purposes, where such decrease is less than about a 1% decrease, or less than about a 2% decrease, or less than about a 3% decrease, or less than about a 4% decrease, or less than about a 5% decrease, or less than about a 6% decrease, or less than about a 7% decrease, or less than about an 8% decrease, or less than about a 9% decrease, or less than about a 10% decrease, or less than about a 15% decrease, or less than about a 20% decrease, or less than about a 25% decrease, or less than about a 30% decrease, or less than about a 35% decrease, or less than about a 40% decrease, or less than about a 45% decrease, or less than about a 50% decrease, or less than about a 60% decrease, or less than about a 70% decrease, or less than about a 80% decrease, or less than about a 90% decrease, or less than about 100% decrease in wetting time, absorption rate, spreading speed, accumulative one-way transport, or overall moisture management capability as compared to a control silk fibroin coating that was not subjected to the selected temperature for drying, curing, wash cycling, and/or heat setting purposes. In some embodiments, “wash cycling” may refer to at least one wash cycle, or at least two wash cycles, or at least three wash cycles, or at least four wash cycles, or at least five wash cycles.

In an embodiment, “substantially modifying” silk fibroin coating performance may be an increase in a selected property of silk fibroin coating, such as wetting time, absorption rate, spreading speed, accumulative one-way transport, or overall moisture management capability as compared to a control silk fibroin coating that was not subjected to the selected temperature for drying, curing, wash cycling, and/or heat setting purposes, where such increase is less than about a 1% increase, or less than about a 2% increase, or less than about a 3% increase, or less than about a 4% increase, or less than about a 5% increase, or less than about a 6% increase, or less than about a 7% increase, or less than about an 8% increase, or less than about a 9% increase, or less than about a 10% increase, or less than about a 15% increase, or less than about a 20% increase, or less than about a 25% increase, or less than about a 30% increase, or less than about a 35% increase, or less than about a 40% increase, or less than about a 45% increase, or less than about a 50% increase, or less than about a 60% increase, or less than about a 70% increase, or less than about a 80% increase, or less than about a 90% increase, or less than about 100% increase in wetting time, absorption rate, spreading speed, accumulative one-way transport, or overall moisture management capability as compared to a control silk fibroin coating that was not subjected to the selected temperature for drying, curing, wash cycling, and/or heat setting purposes. In some embodiments, “wash cycling” may refer to at least one wash cycle, or at least two wash cycles, or at least three wash cycles, or at least four wash cycles, or at least five wash cycles.

In some embodiments, the SFS coated article may be subjected to heat setting in order to set one or more dyes that may be applied to the SFS coated article in order to permanently set the one or more dyes on the SFS coated article. In some embodiments, the SFS coated article may be heat setting resistant, wherein the SFS coating on the SFS coated article may resist a heat setting temperature of greater than about 100° C., or greater than about 110° C., or greater than about 120° C., or greater than about 130° C., or greater than about 140° C., or greater than about 150° C., or greater than about 160° C., or greater than about 170° C., or greater than about 180° C., or greater than about 190° C., or greater than about 200° C., or greater than about 210° C., or greater than about 220° C. In some embodiments, the selected temperature is less than about 100° C., or less than about 110° C., or less than about 120° C., or less than about 130° C., or less than about 140° C., or less than about 150° C., or less than about 160° C., or less than about 170° C., or less than about 180° C., or less than about 190° C., or less than about 200° C., or less than about 210° C., or less than about 220° C.

In an embodiment, a material coated by the silk fibroin coating as described herein may partially dissolved or otherwise partially incorporated within a portion of the material after the silk fibroin coated material is subjected to heating and/or curing as described herein. Without being limited to any one theory of the disclosure, where the silk fibroin coated material is heated to greater than about the glass transition temperature (Tg) for the material that is coated, the silk fibroin coating may become partially dissolved or otherwise partially incorporated within a portion of the material. In some embodiments, a material coated by the silk fibroin coating as described herein may be sterile or may be sterilized to provide a sterilized silk fibroin coated material. Alternatively, or in addition thereto, the methods described herein may include a sterile SFS prepared from sterile silk fibroin.

In some embodiments, the fabric constructions that are compatible with the processes of the disclosure include woven fabrics, knitted fabrics, and non-woven fabrics.

In some embodiments, the coating pattern provided by the processes of the disclosure include one side coating, two side coating, and/or throughout coating.

In some embodiments, the equipment manufacturers that are capable of producing equipment configured to continuously coat SFS on textiles include, but are not limited to, Aigle, Amba Projex, Bombi, Bruckner, Cavitec, Crosta, Dienes Apparatebau, Eastsign, Europlasma, Fermor, Fontanet, Gaston Systems, Hansa Mixer, Harish, Has Group, Icomatex, Idealtech, Interspare, Isotex, Klieverik, KTP, M P, Mageba, Mahr Feinpruef, Matex, Mathis, Menzel LP, Meyer, Monforts, Morrison Textile, Mtex, Muller Frick, Muratex Textile, Reliant Machinery, Rollmac, Salvade, Sandvik Tps, Santex, Chmitt-Machinen, Schott & Meissner, Sellers, Sicam, Siltex, Starlinger, Swatik Group India, Techfull, TMT Manenti, Unitech Textile Machinery, Weko, Willy, Wumag Texroll, Yamuna, Zappa, and Zimmer Austria.

In some embodiments, the equipment manufactures that are capable of producing equipment configured to dry SFS coated on textiles include, but are not limited to, Alea, Alkan Makina, Anglada, Atac Makina, Bianco, Bruckner, Campen, CHTC, CTMTC, Dilmenler, Elteksmak, Erbatech, Fontanet, Harish, Icomatex, Ilsung, Inspiron, Interspare, Master, Mathis, Monfongs, Monforts, Salvade, Schmitt-Maschinen, Sellers, Sicam, Siltex, Swastik Group India, Tacome, Tubetex, Turbang, Unitech Textile Machinery, and Yamuna.

### Silk Protein Fragments in Collagen Boosting Compositions and Methods Thereof

In further embodiments, disclosure provides a method for collagen expression in a subject in need thereof, comprising administering to the subject a composition comprising silk fibroin fragments, or without limitation any other silk protein fragments described herein, including modified silk fragments disclosed herein, e.g., and without limitation, fibroin peptides and/or protein fragments wherein at least one of the amino acids is modified, substituted, or replaced as disclosed herein, having an average weight average molecular weight selected from between about 1 kDa and about 5 kDa, between about 5 kDa and about 10 kDa, between about 6 kDa and about 17 kDa, between about 10 kDa and about 15 kDa, between about 15 kDa and about 20 kDa, between about 14 kDa and about 30 kDa, between about 17 kDa and about 39 kDa, between about 20 kDa and about 25 kDa, between about 25 kDa and about 30 kDa, between about 30 kDa and about 35 kDa, between about 35 kDa and about 40 kDa, between about 39 kDa and about 54 kDa, between about 39 kDa and about 80 kDa, between about 40 kDa and about 45 kDa, between about 45 kDa and about 50 kDa, between about 60 kDa and about 100 kDa, and between about 80 kDa and about 144 kDa, and a polydispersity between 1 and about 5. Any other molecular weight, molecular weight range, and polydispersity of silk fibroin fragments, or without limitation any other silk protein fragments, described herein, can be used in the methods and compositions of the disclosure.

In some embodiments, the composition further comprises 0 to 500 ppm lithium bromide. In some embodiments, the composition further comprises 0 to 500 ppm sodium carbonate. In some embodiments, the silk fibroin fragments, or without limitation any other silk protein fragments described herein, have a polydispersity between 1 and about 1.5. In some embodiments, the silk fibroin fragments, or without limitation any other silk protein fragments described herein, have a polydispersity between about 1.5 and about 2.0. In some embodiments, the silk fibroin fragments, or without limitation any other silk protein fragments described herein, have a polydispersity between about 1.5 and about 3.0. In some embodiments, the silk fibroin fragments, or without limitation any other silk protein fragments described herein, have a polydispersity between about 2.0 and about 2.5. In some embodiments, the silk fibroin fragments, or without limitation any other silk protein fragments described herein, have a polydispersity between about 2.5 and about 3.0. In some embodiments, the silk fibroin fragments, or without limitation any other silk protein fragments described herein, are present in the composition at about 0.001 wt. % to about 10.0 wt. % relative to the total weight of the composition. In some embodiments, the composition further comprises about 0.001% (w/w) to about 10% (w/w) sericin relative to the total weight of the composition. In some embodiments, the composition further comprises about 0.001% (w/w) to about 10% (w/w) sericin relative to the silk fibroin fragments, or without limitation any other silk protein fragments described herein. In some embodiments, the silk fibroin fragments, or without limitation any other silk protein fragments described herein, do not spontaneously or gradually gelate and do not visibly change in color or turbidity when in an aqueous solution for at least 10 days prior to formulation into the composition. In some embodiments, the silk fibroin fragments, or without limitation any other silk protein fragments described herein, are present in the composition at about 0.01 wt. % to about 10.0 wt. % relative to the total weight of the composition. In some embodiments, the silk fibroin fragments, or without limitation any other silk protein fragments described herein, are present in the composition at about 0.01 wt. % to about 1.0 wt. % relative to the total weight of the composition. In some embodiments, the silk fibroin fragments, or without limitation any other silk protein fragments described herein, are present in the composition at about 1.0 wt. % to about 2.0 wt. % relative to the total weight of the composition. In some embodiments, the silk fibroin fragments, or without limitation any other silk protein fragments described herein, are present in the composition at about 2.0 wt. % to about 3.0 wt. % relative to the total weight of the composition. In some embodiments, the silk fibroin fragments, or without limitation any other silk protein fragments described herein, are present in the composition at about 3.0 wt. % to about 4.0 wt. % relative to the total weight of the composition. In some embodiments, the silk fibroin fragments, or without limitation any other silk protein fragments described herein, are present in the composition at about 4.0 wt. % to about 5.0 wt. % relative to the total weight of the composition. In some embodiments, the silk fibroin fragments, or without limitation any other silk protein fragments described herein, are present in the composition at about 5.0 wt. % to about 6.0 wt. % relative to the total weight of the composition.

In some embodiments, collagen expression is increased over a base level by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, collagen expression is increased over a base level by about 101%, about 102%, about 103%, about 104%, about 105%, about 106%, about 107%, about 108%, about 109%, about 110%, about 111%, about 112%, about 113%, about 114%, about 115%, about 116%, about 117%, about 118%, about 119%, about 120%, about 121%, about 122%, about 123%, about 124%, about 125%, about 126%, about 127%, about 128%, about 129%, about 130%, about 131%, about 132%, about 133%, about 134%, about 135%, about 136%, about 137%, about 138%, about 139%, about 140%, about 141%, about 142%, about 143%, about 144%, about 145%, about 146%, about 147%, about 148%, about 149%, about 150%, about 151%, about 152%, about 153%, about 154%, about 155%, about 156%, about 157%, about 158%, about 159%, about 160%, about 161%, about 162%, about 163%, about 164%, about 165%, about 166%, about 167%, about 168%, about 169%, about 170%, about 171%, about 172%, about 173%, about 174%, about 175%, about 176%, about 177%, about 178%, about 179%, about 180%, about 181%, about 182%, about 183%, about 184%, about 185%, about 186%, about 187%, about 188%, about 189%, about 190%, about 191%, about 192%, about 193%, about 194%, about 195%, about 196%, about 197%, about 198%, about 199%, or about 200%.

In some embodiments, collagen expression is increased over a base level by about 225%, about 250%, about 275%, about 300%, about 325%, about 350%, about 375%, about 400%, about 425%, about 450%, about 475%, about 500%, about 525%, about 550%, about 575%, about 600%, about 625%, about 650%, about 675%, about 700%, about 725%, about 750%, about 775%, about 800%, about 825%, about 850%, about 875%, about 900%, about 925%, about 950%, about 975%, or about 1000%.

In some embodiments, the methods of treatment provided include one or more of administering a composition of the disclosure before, after, or during a laser treatment, administering a composition of the disclosure before, after, or during a skin peel, administering a composition of the disclosure before, after, or during a radiation treatment. In some embodiments, the methods of treatment provided include one or more of administering a composition of the disclosure to treat a burn, including without limitation any type of burn (e.g., thermic burn, sunburn, fire burn, hot liquid burn, radiation burn, chemical burn, and the like). In some embodiments, the methods of treatment provided include one or more of administering a composition of the disclosure to treat a burn, including without limitation a first-, second-, or third-degree burn. In some embodiments, the methods of treatment provided include one or more of administering a composition of the disclosure for treating a skin condition due to aging.

Following are non-limiting examples of suitable ranges for various parameters in and for preparation of the silk solutions of the present disclosure, including modified silk fragments disclosed herein, e.g., and without limitation, fibroin peptides and/or protein fragments wherein at least one of the amino acids is modified, substituted, or replaced as disclosed herein. The silk solutions of the present disclosure may include one or more, but not necessarily all, of these parameters and may be prepared using various combinations of ranges of such parameters.

In an embodiment, the percent silk in the solution is, without limitation, less than 30 wt. %. In an embodiment, the percent silk in the solution is less than 25 wt. %. In an embodiment, the percent silk in the solution is less than 20 wt. %. In an embodiment, the percent silk in the solution is less than 19 wt. %. In an embodiment, the percent silk in the solution is less than 18 wt. %. In an embodiment, the percent silk in the solution is less than 17 wt. %. In an embodiment, the percent silk in the solution is less than 16 wt. %. In an embodiment, the percent silk in the solution is less than 15 wt. %. In an embodiment, the percent silk in the solution is less than 14 wt. %. In an embodiment, the percent silk in the solution is less than 13 wt. %. In an embodiment, the percent silk in the solution is less than 12 wt. %. In an embodiment, the percent silk in the solution is less than 11 wt. %. In an embodiment, the percent silk in the solution is less than 10 wt. %. In an embodiment, the percent silk in the solution is less than 9 wt. %. In an embodiment, the percent silk in the solution is less than 8 wt. %. In an embodiment, the percent silk in the solution is less than 7 wt. %.

In an embodiment, the percent silk in the solution is less than 6 wt. %. In an embodiment, the percent silk in the solution is less than 5 wt. %. In an embodiment, the percent silk in the solution is less than 4 wt. %. In an embodiment, the percent silk in the solution is less than 3 wt. %. In an embodiment, the percent silk in the solution is less than 2 wt. %. In an embodiment, the percent silk in the solution is less than 1 wt. %. In an embodiment, the percent silk in the solution is less than 0.9 wt. %. In an embodiment, the percent silk in the solution is less than 0.8 wt. %. In an embodiment, the percent silk in the solution is less than 0.7 wt. %. In an embodiment, the percent silk in the solution is less than 0.6 wt. %. In an embodiment, the percent silk in the solution is less than 0.5 wt. %. In an embodiment, the percent silk in the solution is less than 0.4 wt. %. In an embodiment, the percent silk in the solution is less than 0.3 wt. %. In an embodiment, the percent silk in the solution is less than 0.2 wt. %. In an embodiment, the percent silk in the solution is less than 0.1 wt. %.

In an embodiment, the percent silk in the solution is, without limitation, greater than 0.1 wt. %. In an embodiment, the percent silk in the solution is greater than 0.2 wt. %. In an embodiment, the percent silk in the solution is greater than 0.3 wt. %. In an embodiment, the percent silk in the solution is greater than 0.4 wt. %. In an embodiment, the percent silk in the solution is greater than 0.5 wt. %. In an embodiment, the percent silk in the solution is greater than 0.6 wt. %. In an embodiment, the percent silk in the solution is greater than 0.7 wt. %. In an embodiment, the percent silk in the solution is greater than 0.8 wt. %. In an embodiment, the percent silk in the solution is greater than 0.9 wt. %. In an embodiment, the percent silk in the solution is greater than 1.0 wt. %. In an embodiment, the percent silk in the solution is greater than 2.0 wt. %. In an embodiment, the percent silk in the solution is greater than 3.0 wt. %. In an embodiment, the percent silk in the solution is greater than 4.0 wt. %. In an embodiment, the percent silk in the solution is greater than 5.0 wt. %. In an embodiment, the percent silk in the solution is greater than 6.0 wt. %. In an embodiment, the percent silk in the solution is greater than 7.0 wt. %. In an embodiment, the percent silk in the solution is greater than 8.0 wt. %. In an embodiment, the percent silk in the solution is greater than 9.0 wt. %. In an embodiment, the percent silk in the solution is greater than 10.0 wt. %. In an embodiment, the percent silk in the solution is greater than 11.0 wt. %. In an embodiment, the percent silk in the solution is greater than 12.0 wt. %. In an embodiment, the percent silk in the solution is greater than 13.0 wt. %. In an embodiment, the percent silk in the solution is greater than 14.0 wt. %. In an embodiment, the percent silk in the solution is greater than 15.0 wt. %. In an embodiment, the percent silk in the solution is greater than 16.0 wt. %. In an embodiment, the percent silk in the solution is greater than 17.0 wt. %. In an embodiment, the percent silk in the solution is greater than 18.0 wt. %. In an embodiment, the percent silk in the solution is greater than 19.0 wt. %. In an embodiment, the percent silk in the solution is greater than 20.0 wt. %. In an embodiment, the percent silk in the solution is greater than 25.0 wt. %.

In an embodiment, the percent silk in the solution ranges, without limitation, from about 0.1 wt. % to about 30.0 wt. %. In an embodiment, the percent silk in the solution ranges from about 0.1 wt. % to about 25.0 wt. %. In an embodiment, the percent silk in the solution ranges from about 0.1 wt. % to about 20.0 wt. %. In an embodiment, the percent silk in the solution ranges from about 0.1 wt. % to about 15.0 wt. %. In an embodiment, the percent silk in the solution ranges from about 0.1 wt. % to about 10.0 wt. %. In an embodiment, the percent silk in the solution ranges from about 0.1 wt. % to about 9.0 wt. %. In an embodiment, the percent silk in the solution ranges from about 0.1 wt. % to about 8.0 wt. %. In an embodiment, the percent silk in the solution ranges from about 0.1 wt. % to about 7.0 wt. %. In an embodiment, the percent silk in the solution ranges from about 0.1 wt. % to about 6.5 wt. %. In an embodiment, the percent silk in the solution ranges from about 0.1 wt. % to about 6.0 wt. %. In an embodiment, the percent silk in the solution ranges from about 0.1 wt. % to about 5.5 wt. %. In an embodiment, the percent silk in the solution ranges from about 0.1 wt. % to about 5.0 wt. %. In an embodiment, the percent silk in the solution ranges from about 0.1 wt. % to about 4.5 wt. %. In an embodiment, the percent silk in the solution ranges from about 0.1 wt. % to about 4.0 wt. %. In an embodiment, the percent silk in the solution ranges from about 0.1 wt. % to about 3.5 wt. %. In an embodiment, the percent silk in the solution ranges from about 0.1 wt. % to about 3.0 wt. %. In an embodiment, the percent silk in the solution ranges from about 0.1 wt. % to about 2.5 wt. %. In an embodiment, the percent silk in the solution ranges from about 0.1 wt. % to about 2.0 wt. %. In an embodiment, the percent silk in the solution ranges from about 0.1 wt. % to about 2.4 wt. %. In an embodiment, the percent silk in the solution ranges from about 0.5 wt. % to about 5.0 wt. %. In an embodiment, the percent silk in the solution ranges from about 0.5 wt. % to about 4.5 wt. %. In an embodiment, the percent silk in the solution ranges from about 0.5 wt. % to about 4.0 wt. %. In an embodiment, the percent silk in the solution ranges from about 0.5 wt. % to about 3.5 wt. %. In an embodiment, the percent silk in the solution ranges from about 0.5 wt. % to about 3.0 wt. %. In an embodiment, the percent silk in the solution ranges from about 0.5 wt. % to about 2.5 wt. %. In an embodiment, the percent silk in the solution ranges from about 1.0 wt. % to about 4.0 wt. %. In an embodiment, the percent silk in the solution ranges from about 1.0 wt. % to about 3.5 wt. %. In an embodiment, the percent silk in the solution ranges from about 1.0 wt. % to about 3.0 wt. %. In an embodiment, the percent silk in the solution ranges from about 1.0 wt. % to about 2.5 wt. %. In an embodiment, the percent silk in the solution ranges from about 1.0 wt. % to about 2.4 wt. %. In an embodiment, the percent silk in the solution ranges from about 1.0 wt. % to about 2 wt. %.

In an embodiment, the percent silk in the solution ranges from about 20.0 wt. % to about 30.0 wt. %. In an embodiment, the percent silk in the solution ranges from about 0.1 wt. % to about 10.0 wt. %. In an embodiment, the percent silk in the solution ranges from about 1.0 wt. % to about 10.0 wt. %. In an embodiment, the percent silk in the solution ranges from about 2 wt. % to about 10.0 wt. %. In an embodiment, the percent silk in the solution ranges from about 0.1 wt. % to about 6.0 wt. %. In an embodiment, the percent silk in the solution ranges from about 6.0 wt. % to about 10.0 wt. %. In an embodiment, the percent silk in the solution ranges from about 6.0 wt. % to about 8.0 wt. %. In an embodiment, the percent silk in the solution ranges from about 6.0 wt. % to about 9.0 wt. %. In an embodiment, the percent silk in the solution ranges from about 10.0 wt. % to about 20.0 wt. %. In an embodiment, the percent silk in the solution ranges from about 11.0 wt. % to about 19.0 wt. %. In an embodiment, the percent silk in the solution ranges from about 12.0 wt. % to about 18.0 wt. %. In an embodiment, the percent silk in the solution ranges from about 13.0 wt. % to about 17.0 wt. %. In an embodiment, the percent silk in the solution ranges from about 14.0 wt. % to about 16.0 wt. %. In an embodiment, the percent silk in the solution is about 1.0 wt. %. In an embodiment, the percent silk in the solution is about 1.5 wt. %. In an embodiment, the percent silk in the solution is about 2.0 wt. %. In an embodiment, the percent silk in the solution is about 2.4 wt. %. In an embodiment, the percent silk in the solution is 3.0 wt. %. In an embodiment, the percent silk in the solution is 3.5 wt. %. In an embodiment, the percent silk in the solution is about 4.0 wt. %. In an embodiment, the percent silk in the solution is about 4.5 wt. %. In an embodiment, the percent silk in the solution is about 5.0 wt. %. In an embodiment, the percent silk in the solution is about 5.5 wt. %. In an embodiment the percent silk in the solution is about 6.0 wt. %. In an embodiment, the percent silk in the solution is about 6.5 wt. %. In an embodiment, the percent silk in the solution is about 7.0 wt. %. In an embodiment, the percent silk in the solution is about 7.5 wt. %. In an embodiment, the percent silk in the solution is about 8.0 wt. %. In an embodiment, the percent silk in the solution is about 8.5 wt. %. In an embodiment, the percent silk in the solution is about 9.0 wt. %. In an embodiment, the percent silk in the solution is about 9.5 wt. %. In an embodiment, the percent silk in the solution is about 10.0 wt.

In an embodiment, the percent sericin in the solution is non-detectable to 30.0 wt. %. In an embodiment, the percent sericin in the solution is non-detectable to 5.0 wt. %. In an embodiment, the percent sericin in the solution is 1.0 wt. %. In an embodiment, the percent sericin in the solution is 2.0 wt. %. In an embodiment, the percent sericin in the solution is 3.0 wt. %. In an embodiment, the percent sericin in the solution is 4.0 wt. %. In an embodiment, the percent sericin in the solution is 5.0 wt. %. In an embodiment, the percent sericin in the solution is 10.0 wt. %. In an embodiment, the percent sericin in the solution is 30.0 wt. %.

In some embodiments, the silk fibroin protein based fragments of the present disclosure are shelf stable (they will not slowly or spontaneously gel when stored in an aqueous solution and there is no aggregation of fragments and therefore no increase in molecular weight over time), from 10 days to 3 years depending on storage conditions, percent silk, and number of shipments and shipment conditions.

Additionally, pH may be altered to extend shelf-life and/or support shipping conditions by preventing premature folding and aggregation of the silk. In an embodiment, the stability of the LiBr-silk fragment solution is 0 to 1 year. In an embodiment, the stability of the LiBr-silk fragment solution is 0 to 2 years. In an embodiment, the stability of the LiBr-silk fragment solution is 0 to 3 years. In an embodiment, the stability of the LiBr-silk fragment solution is 0 to 4 years. In an embodiment, the stability of the LiBr-silk fragment solution is 0 to 5 years. In an embodiment, the stability of the LiBr-silk fragment solution is 1 to 2 years. In an embodiment, the stability of the LiBr-silk fragment solution is 1 to 3 years. In an embodiment, the stability of the LiBr-silk fragment solution is 1 to 4 years. In an embodiment, the stability of the LiBr-silk fragment solution is 1 to 5 years. In an embodiment, the stability of the LiBr-silk fragment solution is 2 to 3 years. In an embodiment, the stability of the LiBr-silk fragment solution is 2 to 4 years. In an embodiment, the stability of the LiBr-silk fragment solution is 2 to 5 years. In an embodiment, the stability of the LiBr-silk fragment solution is 3 to 4 years. In an embodiment, the stability of the LiBr-silk fragment solution is 3 to 5 years. In an embodiment, the stability of the LiBr-silk fragment solution is 4 to 5 years.

In an embodiment, the stability of a composition of the present disclosure is 10 days to 6 months. In an embodiment, the stability of a composition of the present disclosure is 6 months to 12 months. In an embodiment, the stability of a composition of the present disclosure is 12 months to 18 months. In an embodiment, the stability of a composition of the present disclosure is 18 months to 24 months. In an embodiment, the stability of a composition of the present disclosure is 24 months to 30 months. In an embodiment, the stability of a composition of the present disclosure is 30 months to 36 months. In an embodiment, the stability of a composition of the present disclosure is 36 months to 48 months. In an embodiment, the stability of a composition of the present disclosure is 48 months to 60 months.

In an embodiment, a composition of the present disclosure includes silk fibroin-based protein fragments having a weight average molecular weight selected from between 1 kDa to 5 kDa. In an embodiment, a composition of the present disclosure includes silk fibroin-based protein fragments having a weight average molecular weight selected from between 5 kDa to 10 kDa. In an embodiment, a composition of the present disclosure includes silk fibroin-based protein fragments having a weight average molecular weight selected from between 10 kDa to 15 kDa. In an embodiment, a composition of the present disclosure includes silk fibroin-based protein fragments having a weight average molecular weight selected from between 15 kDa to 20 kDa. In an embodiment, a composition of the present disclosure includes silk fibroin-based protein fragments having a weight average molecular weight selected from between 20 kDa to 25 kDa. In an embodiment, a composition of the present disclosure includes silk fibroin-based protein fragments having a weight average molecular weight selected from between 25 kDa to 30 kDa. In an embodiment, a composition of the present disclosure includes silk fibroin-based protein fragments having a weight average molecular weight selected from between 30 kDa to 35 kDa. In an embodiment, a composition of the present disclosure includes silk fibroin-based protein fragments having a weight average molecular weight selected from between 35 kDa to 40 kDa. In an embodiment, a composition of the present disclosure includes silk fibroin-based protein fragments having a weight average molecular weight selected from between 40 kDa to 45 kDa. In an embodiment, a composition of the present disclosure includes silk fibroin-based protein fragments having a weight average molecular weight selected from between 45 kDa to 50 kDa. In an embodiment, a composition of the present disclosure includes silk fibroin-based protein fragments having a weight average molecular weight selected from between 50 kDa to 55 kDa. In an embodiment, a composition of the present disclosure includes silk fibroin-based protein fragments having a weight average molecular weight selected from between 55 kDa to 60 kDa. In an embodiment, a composition of the present disclosure includes silk fibroin-based protein fragments having a weight average molecular weight selected from between 60 kDa to 65 kDa. In an embodiment, a composition of the present disclosure includes silk fibroin-based protein fragments having a weight average molecular weight selected from between 65 kDa to 70 kDa. In an embodiment, a composition of the present disclosure includes silk fibroin-based protein fragments having a weight average molecular weight selected from between 70 kDa to 75 kDa. In an embodiment, a composition of the present disclosure includes silk fibroin-based protein fragments having a weight average molecular weight selected from between 75 kDa to 80 kDa. In an embodiment, a composition of the present disclosure includes silk fibroin-based protein fragments having a weight average molecular weight selected from between 80 kDa to 85 kDa. In an embodiment, a composition of the present disclosure includes silk fibroin-based protein fragments having a weight average molecular weight selected from between 85 kDa to 90 kDa. In an embodiment, a composition of the present disclosure includes silk fibroin-based protein fragments having a weight average molecular weight selected from between 90 kDa to 95 kDa. In an embodiment, a composition of the present disclosure includes silk fibroin-based protein fragments having a weight average molecular weight selected from between 95 kDa to 100 kDa. In an embodiment, a composition of the present disclosure includes silk fibroin-based protein fragments having a weight average molecular weight selected from between 100 kDa to 105 kDa. In an embodiment, a composition of the present disclosure includes silk fibroin-based protein fragments having a weight average molecular weight selected from between 105 kDa to 110 kDa. In an embodiment, a composition of the present disclosure includes silk fibroin-based protein fragments having a weight average molecular weight selected from between 110 kDa to 115 kDa. In an embodiment, a composition of the present disclosure includes silk fibroin-based protein fragments having a weight average molecular weight selected from between 115 kDa to 120 kDa. In an embodiment, a composition of the present disclosure includes silk fibroin-based protein fragments having a weight average molecular weight selected from between 120 kDa to 125 kDa. In an embodiment, a composition of the present disclosure includes silk fibroin-based protein fragments having a weight average molecular weight selected from between 125 kDa to 130 kDa. In an embodiment, a composition of the present disclosure includes silk fibroin-based protein fragments having a weight average molecular weight selected from between 130 kDa to 135 kDa. In an embodiment, a composition of the present disclosure includes silk fibroin-based protein fragments having a weight average molecular weight selected from between 135 kDa to 140 kDa. In an embodiment, a composition of the present disclosure includes silk fibroin-based protein fragments having a weight average molecular weight selected from between 140 kDa to 145 kDa. In an embodiment, a composition of the present disclosure includes silk fibroin-based protein fragments having a weight average molecular weight selected from between 145 kDa to 150 kDa. In an embodiment, a composition of the present disclosure includes silk fibroin-based protein fragments having a weight average molecular weight selected from between 150 kDa to 155 kDa. In an embodiment, a composition of the present disclosure includes silk fibroin-based protein fragments having a weight average molecular weight selected from between 155 kDa to 160 kDa. In an embodiment, a composition of the present disclosure includes silk fibroin-based protein fragments having a weight average molecular weight selected from between 160 kDa to 165 kDa. In an embodiment, a composition of the present disclosure includes silk fibroin-based protein fragments having a weight average molecular weight selected from between 165 kDa to 170 kDa. In an embodiment, a composition of the present disclosure includes silk fibroin-based protein fragments having a weight average molecular weight selected from between 170 kDa to 175 kDa. In an embodiment, a composition of the present disclosure includes silk fibroin-based protein fragments having a weight average molecular weight selected from between 175 kDa to 180 kDa. In an embodiment, a composition of the present disclosure includes silk fibroin-based protein fragments having a weight average molecular weight selected from between 180 kDa to 185 kDa. In an embodiment, a composition of the present disclosure includes silk fibroin-based protein fragments having a weight average molecular weight selected from between 185 kDa to 190 kDa. In an embodiment, a composition of the present disclosure includes silk fibroin-based protein fragments having a weight average molecular weight selected from between 190 kDa to 195 kDa. In an embodiment, a composition of the present disclosure includes silk fibroin-based protein fragments having a weight average molecular weight selected from between 195 kDa to 200 kDa. In an embodiment, a composition of the present disclosure includes silk fibroin-based protein fragments having a weight average molecular weight selected from between 200 kDa to 205 kDa. In an embodiment, a composition of the present disclosure includes silk fibroin-based protein fragments having a weight average molecular weight selected from between 205 kDa to 210 kDa. In an embodiment, a composition of the present disclosure includes silk fibroin-based protein fragments having a weight average molecular weight selected from between 210 kDa to 215 kDa. In an embodiment, a composition of the present disclosure includes silk fibroin-based protein fragments having a weight average molecular weight selected from between 215 kDa to 220 kDa. In an embodiment, a composition of the present disclosure includes silk fibroin-based protein fragments having a weight average molecular weight selected from between 220 kDa to 225 kDa. In an embodiment, a composition of the present disclosure includes silk fibroin-based protein fragments having a weight average molecular weight selected from between 225 kDa to 230 kDa. In an embodiment, a composition of the present disclosure includes silk fibroin-based protein fragments having a weight average molecular weight selected from between 230 kDa to 235 kDa. In an embodiment, a composition of the present disclosure includes silk fibroin-based protein fragments having a weight average molecular weight selected from between 235 kDa to 240 kDa. In an embodiment, a composition of the present disclosure includes silk fibroin-based protein fragments having a weight average molecular weight selected from between 240 kDa to 245 kDa. In an embodiment, a composition of the present disclosure includes silk fibroin-based protein fragments having a weight average molecular weight selected from between 245 kDa to 250 kDa. In an embodiment, a composition of the present disclosure includes silk fibroin-based protein fragments having a weight average molecular weight selected from between 250 kDa to 255 kDa. In an embodiment, a composition of the present disclosure includes silk fibroin-based protein fragments having a weight average molecular weight selected from between 255 kDa to 260 kDa. In an embodiment, a composition of the present disclosure includes silk fibroin-based protein fragments having a weight average molecular weight selected from between 260 kDa to 265 kDa. In an embodiment, a composition of the present disclosure includes silk fibroin-based protein fragments having a weight average molecular weight selected from between 265 kDa to 270 kDa. In an embodiment, a composition of the present disclosure includes silk fibroin-based protein fragments having a weight average molecular weight selected from between 270 kDa to 275 kDa. In an embodiment, a composition of the present disclosure includes silk fibroin-based protein fragments having a weight average molecular weight selected from between 275 kDa to 280 kDa. In an embodiment, a composition of the present disclosure includes silk fibroin-based protein fragments having a weight average molecular weight selected from between 280 kDa to 285 kDa. In an embodiment, a composition of the present disclosure includes silk fibroin-based protein fragments having a weight average molecular weight selected from between 285 kDa to 290 kDa. In an embodiment, a composition of the present disclosure includes silk fibroin-based protein fragments having a weight average molecular weight selected from between 290 kDa to 295 kDa. In an embodiment, a composition of the present disclosure includes silk fibroin-based protein fragments having a weight average molecular weight selected from between 295 kDa to 300 kDa. In an embodiment, a composition of the present disclosure includes silk fibroin-based protein fragments having a weight average molecular weight selected from between 300 kDa to 305 kDa. In an embodiment, a composition of the present disclosure includes silk fibroin-based protein fragments having a weight average molecular weight selected from between 305 kDa to 310 kDa. In an embodiment, a composition of the present disclosure includes silk fibroin-based protein fragments having a weight average molecular weight selected from between 310 kDa to 315 kDa. In an embodiment, a composition of the present disclosure includes silk fibroin-based protein fragments having a weight average molecular weight selected from between 315 kDa to 320 kDa. In an embodiment, a composition of the present disclosure includes silk fibroin-based protein fragments having a weight average molecular weight selected from between 320 kDa to 325 kDa. In an embodiment, a composition of the present disclosure includes silk fibroin-based protein fragments having a weight average molecular weight selected from between 325 kDa to 330 kDa. In an embodiment, a composition of the present disclosure includes silk fibroin-based protein fragments having a weight average molecular weight selected from between 330 kDa to 335 kDa. In an embodiment, a composition of the present disclosure includes silk fibroin-based protein fragments having a weight average molecular weight selected from between 350 kDa to 340 kDa. In an embodiment, a composition of the present disclosure includes silk fibroin-based protein fragments having a weight average molecular weight selected from between kDa 340 to 345 kDa. In an embodiment, a composition of the present disclosure includes silk fibroin-based protein fragments having a weight average molecular weight selected from between kDa 345 to 350 kDa.

In an embodiment, a composition of the silk fibroin-based protein fragments in this disclosure has a polydispersity selected from between about 1 to about 5.0, In an embodiment, a composition of the silk fibroin-based protein fragments has a polydispersity selected from between about 1.5 to about 3.0. In an embodiment, a composition of the silk fibroin-based protein fragments has a polydispersity selected from between about 1 to about 1.5. In an embodiment, a composition of the silk fibroin-based protein fragments has a polydispersity selected from between about 1.5 to about 2.0. In an embodiment, a composition of the silk fibroin-based protein fragments has a polydispersity selected from between about 2.0 to about 2.5. In an embodiment, a composition of the silk fibroin-based protein fragments, has a polydispersity selected from between about is 2.0 to about 3.0. In an embodiment, a composition of the silk fibroin-based protein fragments has a polydispersity selected from between about is 2.5 to about 3.0.

In some embodiments, silk fibroin protein fragments useful for applications in collagen stimulating compositions and methods of making and using thereof also include an aqueous gel of the silk fibroin protein fragments. The gelation of silk fibroin protein fragment solutions may be induced by sonication, vortex, heating, solvent treatment (e.g. methanol, ethanol), electrogelation, ultrasonication, chemicals (e.g. vitamin C), or the like.

Silk peptide is an extract from natural silk fibroin hydrolysate. Silk peptide exhibits pearl luster and silky feel when incorporated into personal care products. The structure of silk peptide is similar to human hair and skin tissue. The silk peptides are serine rich polypeptides having 10 or more amino acid residues and weight average molecular weights as described herein. In some embodiments, the silk peptide extract can be easily absorbed by skin, for example human skin, provide nutrients for skin, and promote the metabolism of skin.

In some embodiments, the collagen stimulating compositions and methods of making and using thereof optionally comprises plant extract that enhances the beneficial effects of silk fibroin protein fragments. In some embodiments, the plant extract is selected from the group consisting of extracts from rice, oat, almond, Camellia Sinensis (green tea) extract, Butyrospermum Parkii (shea butter), coconut, papaya, mango, peach, lemon, wheat, rosemary, apricot, algae, grapefruit, sandalwood, lime, orange, Acacia concinna, Butea parviflora, Butea superb, Butea frondosa, Campanulata (fire tulip), Adansonia Digitata (Baobab), Phoenix Dactylifera (date), Hibiscus Sabdarifa (hibiscus), Aframomum Melegueta (African pepper), Khaya Senegalensis (mahogany wood), Tamarindus Indica (tamarind, or curcumin), Cyperus Papyrus (papyrus), Ageratum spp., birch, burdock, horsetail, lavender, marjoram, nettle, tail cat, thyme, oak bark, echinacea, stinging nettle, witch hazel, hops, henna, chamomile, whitethorn, lime-tree blossom, almond, pine needles, horse chestnut, juniper, kiwi, melon, mallow, cuckoo flower, wild thyme, yarrow, melissa, rest harrow, coltsfoot, marshmallow, rice meristem, moringa, ginseng and ginger root, aloe vera, aloe barbadensis leaf extract, Lavandula angustifolia (lavender) flower extract, sambucus nigra (elderberry) fruit extract, phoenix dactylifera (date) seed extract, avandula stoechas (spanish lavender) extract, spiraea ulmaria (meadowsweet) leave extract, chamomilla recutita (chamomile) leaf extract, and Symphytum oficinale (comfrey) leaf extract and combination thereof. The extracts of these plants are obtained from seeds, roots, stem, leaves, flowers, bark, fruits, and/or whole plant.

In some embodiments, the plant extract is presented in the collagen stimulating compositions and methods of making and using thereof at a weight percent ranging from about 0.001 wt. % to about 10.0 wt. % by the total weight of the composition. In some embodiments, the plant extract is presented in the collagen stimulating compositions and methods of making and using thereof at a weight percent ranging from about 0.005 wt. % to about 5.0 wt. % by the total weight of the composition. In some embodiments, the plant extract is presented in the collagen stimulating compositions and methods of making and using thereof at a weight percent ranging from about 0.01 wt. % to about 2.0 wt. % by the total weight of the composition. In some embodiments, the plant extract is presented in the collagen stimulating compositions and methods of making and using thereof at a weight percent ranging from 0.0045 wt. % to 0.0055 wt. % by the total weight of the composition.

In some embodiments, the collagen stimulating compositions and methods of making and using thereof optionally comprises a UV filter that absorbs ultraviolet light of wavelengths between 290 to 329 nm. In some embodiments, the collagen stimulating compositions and methods of making and using thereof include an UV filter selected from the group consisting of para-aminobenzoic acid, ethyl para-aminobenzoate, amyl para-aminobenzoate, octyl para-aminobenzoate, ethylene glycol salicylate, phenyl salicylate, octyl salicylate, benzyl salicylate, butylphenyl salicylate, homomenthyl salicylate, benzyl cinnamate, 2-ethoxyethyl para-methoxycinnamate, octyl para-methoxycinnamate, glyceryl mono(2-ethylhexanoate) dipara-methoxycinnamate, isopropyl para-methoxycinnamate, diisopropyl-diisopropylcinnamic acid ester mixtures, urocanic acid, ethyl urocanate, hydroxymethoxybenzophenone, hydroxymethoxybenzophenonesulfonic acid and salts thereof, dihydroxymethoxybenzophenone, sodium dihydroxymethoxybenzophenonedisulfonate, dihydroxybenzophenone, tetrahydroxybenzophenone, 4-tert-butyl-4′-methoxydibenzoylmethane, 2,4,6-trianilino-p-(carbo-2′-ethylhexyl-1′-oxy)-1,3,5-triazine, and 2-(2-hydroxy-5-methylphenyl)benzotriazole. In some embodiments, the water soluble ultraviolet absorbent selected from the group consisting of 2-ethylhexyl-p-methoxycinnamate, 4-tert-butyl-4′-methoxydibenzoylmethane, octocrylene, 2,4-bis-[{4-(2-ethylhexyloxy)-2-hydroxy}-phenyl]-6-(4-methoxyphenyl)-1,3,5-triazine, methylene bis-benzotriazolyl tetramethylbutylphenol, 2,4,6-tris-[4-(2-ethylhexyloxycarbonyl)anilino]-1,3,5-triazine, diethylamino hydroxybenzoyl hexyl benzoate, oxybenzone, 2,2′-dihydroxy-4,4′-dimethoxy benzophenone, and combination thereof.

In some embodiments, the UV filter is selected from the group consisting of butyl methoxydibenzoylmethane, ethylhexyl methoxycinnamate, ethylhexyl salicylate, octocrylene, ethylhexyl methoxycinnamate, isoamyl-p-methoxycinnamate, ethylhexyltriazone, diethylhexyl butamido triazone, methylene bis-benzotriazolyl tetramethylbutylphenol, disodium phenyl dibenzimidazole tetrasulfonate, bis-ethylhexyloxyphenol methoxyphenyl triazine, benzophenone-3, and combination thereof.

In some embodiments, the collagen stimulating compositions and methods of making and using thereof comprises an inorganic pigment as UV filters selected from TiO₂, SiO₂, Fe₂O₃, ZrO₂, MnO, Al₂O₃, and combination thereof.

In some embodiments, the UV filter is presented in the composition at a weight percent ranging from about 0.001 wt. % to about 20.0 wt. % by the total weight of the collagen boosting composition. In some embodiments, the UV filter is presented in the composition at a weight percent ranging from about 0.01 wt. % to about 10.0 wt. % by the total weight of the composition. In some embodiments, the UV filter is presented in the composition at a weight percent ranging from about 0.05 wt. % to about 8.0 wt. % by the total weight of the composition.

In some embodiments, the collagen stimulating compositions and methods of making and using thereof optionally comprises an emollient selected from the group consisting of a hydrocarbon oil, a hydrocarbon wax, a silicone oil, an acetoglyceride ester, an ethoxylated glyceride, an alkyl ester of a fatty acid, an alkenyl ester of a fatty acid, a fatty acid, a fatty alcohol, a fatty alcohol ether, an ether-ester, lanolin, a lanolin derivative, a polyhydric alcohol, a polyether derivative, a polyhydric ester, a wax ester, a beeswax derivative, a vegetable wax, a natural or essential oil, a phospholipid, a sterol, an amide, and combination thereof.

In some embodiments, the emollients incorporated in the collagen stimulating compositions and methods of making and using thereof comprise one or more of (1) hydrocarbon oils and waxes, e.g., mineral oil, petrolatum, paraffin, ozokerite, microcrystalline wax, polyethylene, squalene, and perhydrosqualene; (2) silicone oils, e.g., dimethyl polysiloxanes, methylphenyl polysiloxanes, water-soluble and alcohol-soluble silicone glycol copolymers; (3) acetoglyceride esters, e.g., acetylated monoglycerides; (4) ethoxylated glycerides, e.g., ethoxylated glyceryl monostearate; (5) alkyl esters of fatty acids having 10 to 20 carbon atoms, e.g., hexyl laurate, isohexyl laurate, isohexyl palmitate, isopropyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, lauryl lactate, myristyl lactate, methyl, isopropyl, butyl esters of fatty acids; (6) alkenyl esters of fatty acids having 10 to 20 carbon atoms, e.g., oleyl myristate, oleyl stearate, and oleyl oleate; (7) fatty acids having 10 to 20 carbon atoms, e.g., pelargonic, lauric, myristic, palmitic, stearic, isostearic, hydroxystearic, oleic, linoleic, ricinoleic, arachidic, behenic, and erucic acids; (8) fatty alcohols having 10 to 20 carbon atoms, e.g., lauryl, myristyl, cetyl, hexadecyl, stearyl, isostearyl, hydroxystearyl, oleyl, ricinoleyl, behenyl, erucyl alcohols, and 2-octyl dodecanol; (9) fatty alcohols ethers, e.g., ethoxylated fatty alcohols of 10 to 20 carbon atoms, lauryl, cetyl, stearyl, isostearyl, oleyl, and cholesterol alcohols having attached thereto from 1 to 50 ethylene oxide groups or 1 to 50 propylene oxide groups; (10) ether-esters, e.g. fatty acid esters of ethoxylated fatty alcohols; (11) lanolin and its derivatives, e.g., lanolin oil, lanolin wax, lanolin alcohols, lanolin fatty acids, isopropyl lanolate, ethoxylated lanolin, ethoxylated lanolin alcohols, ethoxylated cholesterol, propoxylated lanolin alcohols, acetylated lanolin, acetylated lanolin alcohols, lanolin alcohols linoleate, lanolin alcohols ricinoleate, acetate of lanolin alcohols ricinoleate, acetate of ethoxylated alcohols-esters, hydrogenolysis of lanolin, ethoxylated hydrogenated lanolin, ethoxylated sorbitol lanolin, and liquid and semisolid lanolin absorption bases; (12) polyhydric alcohols and polyether derivatives, e.g., propylene glycol, dipropylene glycol, polypropylene glycols 2000 and 4000, polyoxyethylene glycols, polyoxypropylene polyoxyethylene glycols, glycerol, sorbitol, ethoxylated sorbitol, hydroxypropyl sorbitol, polyethylene glycols 200-6000, methoxy polyethylene glycols 350, 550, 750, 2000 and 5000, poly[ethylene oxide]homopolymers (weight average molecular weight of 100,000-5,000,000 Da), polyalkylene glycols and derivatives, hexylene glycol (2-methyl-2,4-pentanediol), 1, 3-butylene glycol, 1,2,6-hexanetriol, ethohexadiol USP (2-ethyl-1,3-hexanediol), C15-C18 vicinal glycol, and polyoxypropylene derivatives of trimethylolpropane; (13) polyhydric alcohol esters, e.g., ethylene glycol mono- and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters, sucrose cocoate, sucrose dilaurate, sucrose distearate, sucrose hexaerucate, sucrose laurate, sucrose myristate, sucrose oleate, sucrose palmitate, sucrose pentaerucate, sucrose polybehenate, sucrose polycottonseedate, sucrose polylaurate, sucrose polylinoleate, sucrose polyoleate, sucrose polypalmate, sucrose polysoyate, sucrose polystearate, sucrose ricinoleate, sucrose stearate, sucrose tetraisostearate, sucrose tribehenate, sucrose tristearat; (14) wax esters, e.g., beeswax, spermaceti, myristyl myristate, and stearyl stearate; (15) beeswax derivatives, e.g., polyoxyethylene sorbitol beeswax which are reaction products of beeswax with ethoxylated sorbitol of varying ethylene oxide content; (16) vegetable waxes, e.g., carnauba and candelilla waxes; (17) natural or essential oils, e.g., citrus oil, non-citrus fruit oil, nut oils, oils having flavors, perfume or scents, canola oil, corn oil, neem oil, olive oil, cottonseed oil, coconut oil, fractionated coconut oil, palm oil, nut oils, safflower oil, sesame oil, soybean oil, peanut oil, almond oil, cashew oil, hazelnut oil, macadamia oil, pecan oil, pine nut oil, pistachio oil, walnut oil, grapefruit seed oil, lemon oil, orange oil, sweet orange oil, tangerine oil, lime oil, mandarin oil, omega 3 oil, flaxseed oil (linseed oil), apricot oil, avocado oil, carrot oil, cocoa butter oil, coconut oil, fractionated coconut oil, hemp oil, papaya seed oil, rice bran oil, shea butter oil, tea tree seed oil, and wheat germ oil, lavender oil, rosemary oil, tung oil, jojoba oil, poppy seed oil, shea butter, castor oil, mango oil, rose hip oil, tall oil chamomile oil, cinnamon oil, citronella oil, eucalyptus oil, fennel seed oil, jasmine oil, juniper berry oil, raspberry seed oil, lavender oil, primrose oil, lemon grass oil, nutmeg oil, patchouli oil, peppermint oil, pine oil, rose oil, rose hip oil, rosemary oil, eucalyptus oil, tea tree oil, rosewood oil, sandalwood oil, sassafras oil, spearmint oil, Ricinus communis (castor) seed oil, wintergreen oil; (18) phospholipids, e.g., lecithin and derivatives; (19) sterols, e.g., cholesterol and cholesterol fatty acid esters; and (20) fatty acid amides, ethoxylated fatty acid amides, and solid fatty acid alkanolamides, (21) lanolin, therbroma cacao (cocoa) seed butter, petrolatum, euphorbia cerifera (candelilla) wax, honey, geraniol, menthol, camphor, cetyl esters, mineral oil, salicylic acid, phenol, palmitoyl isoleucine,

In some embodiments, the collagen stimulating compositions and methods of making and using thereof optionally comprises a moisturizer selected from the group consisting of water-soluble, low molecular weight moisturizers, fat-soluble, low molecular weight moisturizers, water-soluble, high molecular weight moisturizers and fat-soluble, high molecular weight moisturizers, humectant, and combination thereof.

In some embodiments, the moisturizer comprises a humectant. As used herein, the term “humectant” refer to a hygroscopic substance used to keep things moist. A humectant attracts and retains the moisture in the air nearby via absorption, drawing the water vapor into or beneath the organism's or object's surface.

In some embodiments, the collagen stimulating compositions and methods of making and using thereof optionally comprises a water-soluble silk fibroin peptide as humectant. The amino peptides derived from the silk fibroin protein fragments can be easily absorbed by skin. In some embodiments, a water-soluble silk fibroin peptide may be added to the composition to give an enhanced after use feeling.

In some embodiments, amino acids derived from the silk fibroin protein fragments may be added to the collagen stimulating compositions and methods of making and using thereof as a conditioning agent (e.g. to exert excellent condition effects such as moist feel, softness, smoothness, gloss).

In some embodiments, the collagen stimulating compositions and methods of making and using thereof may comprise one or more additional humectant selected from the group consisting of honey, aloe vera, aloe vera leaf juice, aloe vera leaf extract, sorbitol, urea, lactic acid, sodium lactate, pyrrolidone carboxylic acid, trehalose, maltitol, alpha-hydroxy acids, sodium pyroglutamate, pyrolidonecarboxylate, N-acetyl-ethanolamine, sodium lactate, isopropanol, polyalkylene glycols (e.g., ethylene glycol, propylene glycol, hexylene glycol, 1,3-butylene glycol, dipropylene glycol, triethylene glycol), 1,3-propanediol, diethylene glycol monoethyl ether, glyceryl coconate, hydroxystearate, myristate, oleate, free and/or uncrosslinked hyaluronic acid or a salt thereof, chondroitin sulfuric acid, phospholipids, collagen, elastin, ceramides, lecithin sorbitol, PEG-4, and combination thereof.

In some embodiments, the collagen stimulating compositions and methods of making and using thereof optionally comprise polyhydric alcohols as moisturizer selected from the group consisting of ethylene glycol, propylene glycol, 1,3 butylene glycol, glycerin, sorbitol, polyethylene glycol, glutamine, mannitol, pyrrolidone-sodium carboxylate, (polymerization degree n=2 or more), polypropylene glycol (polymerization degree n=2 or more), polyglycerin (polymerization degree n=2 or more), lactic acid, lactate, and combination thereof.

In some embodiments, the collagen stimulating compositions and methods of making and using thereof optionally comprise fat-soluble, low molecular weight moisturizers selected from the group consisting of cholesterol and cholesterol ester. In some embodiments, the composition optionally comprises water-soluble, high molecular weight moisturizers selected from the group consisting of carboxyvinyl polymers, polyaspartate, tragacanth, xanthane gum, methyl cellulose, hydroxymethyl cellulose, hydroxyethyl cellulose, hydroxypropyl cellulose, carboxymethyl cellulose, water-soluble chitin, chitosan and dextrin. In some embodiments, the composition optionally comprises fat-soluble, high molecular weight moisturizers selected from the group consisting of polyvinylpyrrolidone-eicosene copolymers, polyvinylpyrrolidone-hexadecene copolymers, nitrocellulose, dextrin fatty acid ester and high molecular silicone.

Additional suitable moisturizers include polymeric moisturizers that are water soluble and/or water swellable in nature. In some embodiments, free and/or uncrosslinked hyaluronic acid, or chitosan is combined with moisturizers to enhance their properties.

In some embodiments, the collagen stimulating compositions and methods of making and using thereof contains moisturizer at about 0.1 wt. % to about 30.0 wt. % by the total weight of the collagen boosting composition. In some embodiments, the composition contains moisturizer at about 0.5 wt. % to about 25.0 wt. % by the total weight of the collagen boosting composition. In some embodiments, the composition contains moisturizer at about 1.0 wt. % to about 20.0 wt. % by the total weight of the composition.

In some embodiments, the collagen stimulating compositions and methods of making and using thereof optionally comprise a particle, wherein the particle may include polymeric particle, mica, silica, mud, and clay. The particles in the collagen stimulating compositions and methods of making and using thereof provide the benefits of smoothness, reduced friction, slippery feel whilst leaving the hair feeling clean, light and airy, and improved texture when spread on the hands and/or hair.

In some embodiments, the collagen stimulating compositions and methods of making and using thereof contains a polymeric particle formed of a polymer selected from the group consisting of an anionic and/or nonionic and/or zwitterionic polymer. In some embodiments, the composition contains a polymeric particle formed of a polymer selected from the group consisting of polystyrene, polyvinylacetate, polydivinylbenzene, polymethylmethacrylate, poly-n-butylacrylate, poly-n-butylmethacrylate, poly-2-ethylhexylmethyacrylate, 6,12-nylon, poyurethanes, epoxy resins, styrene/vinyl acetate copolymers, styrene/trimethylaminoethyl methacrylate chloride copolymers, and combinations thereof.

In some embodiments, the collagen stimulating compositions and methods of making and using thereof contains a cationically polymeric particle formed of a hydrophobic polymer selected from the group consisting of polyethylene homopolymers, ethylene-acrylic acid copolymer, polyamide polymer having a molecular weight in the range of from about 6,000 Da to about 12,000 Da, polyethylene-vinyl acetate copolymer, silicone-synthetic wax copolymer, silicone-natural wax copolymer, candelilla-silicone copolymer, ozokerite-silicone copolymer, synthetic paraffin wax-silicone copolymer, and combinations thereof.

In some embodiments, the collagen stimulating compositions and methods of making and using thereof contains swollen polymer particles for depositing discrete particles. In some embodiments, the swollen polymer particles are selected from the group consisting of particulate silicone polymers and surface-alkylated spherical silicon particles. In some embodiments, the silicone polymers forming the swollen polymer particles are selected from the group consisting of polydiorganosiloxanes, polymonoorganosiloxanes, and cross-linked polydimethyl siloxanes, crosslinked polymonomethyl siloxanes optionally having end groups including hydroxyl or methyl, and crosslinked polydimethyl siloxane (DC 2-9040 silicone fluid by Dow Corning). The polydisorganosiloxanes are preferably derived from suitable combinations of R₃SiO_{0.5} repeating units and R₂SiO repeating units. The polymonoorganosiloxanes are derived from R₁SiO_{1.5}. Each R independently represents an alkyl, alkenyl (e.g. vinyl), alkaryl, aralkyl, or aryl (e.g. phenyl) group. In some embodiments, R is a methyl group.

In some embodiments, the polymeric particles are nanoparticles having a median particle size of less than 1000 nm. In some embodiments, the polymeric particles have a median particle size of about 5 nm to about 600 nm. In some embodiments, the polymeric particles have a median particle size of about 10 nm to about 500 nm. In some embodiments, the polymeric particles have a median particle size of about 10 nm to about 400 nm. In some embodiments, the polymeric particles have a median particle size of about 20 nm to about 300 nm. In some embodiments, the polymeric particles have a median particle size of about 50 nm to about 600 nm.

In some embodiments, the collagen stimulating compositions and methods of making and using thereof contains clay particles forming a dispersion or a suspension in the dermatologically acceptable carrier as disclosed herein. Throughout this specification, the term “clay” is intended to mean fine-grained earthy materials that become plastic when mixed with water. The clay may be a natural, synthetic or chemically modified clay. Clays include hydrous aluminum silicates which contain impurities, e.g. potassium, sodium, magnesium, or iron in small amounts.

In one embodiment, the clay is a material containing from 38.8% to 98.2% of SiO₂ and from 0.3% to 38.0% of Al₂O₃, and further contains one or more of metal oxides selected from Fe₂O₃, CaO, MgO, TiO₂, ZrO₂, Na₂O and K₂O. In some embodiments, the clay has a layered structure comprising hydrous sheets of octahedrally coordinated aluminum, magnesium or iron, or of tetrahedrally coordinated silicon.

In one embodiment, the clay is selected from the group consisting of kaolin, talc, 2:1 phyllosilicates, 1:1 phyllosilicates, smectite, bentonite, montmorillonites (also known as bentonites), hectorites, volchonskoites, nontronites, saponites, beidelites, sauconites, and mixtures thereof. In one embodiment, the clay is kaolin or bentonite. In some embodiments, the clay is a synthetic hectorite. In another embodiment, the clay is a bentonite.

In some embodiments, the clays have a cation exchange capacity of from about 0.7 meq/100 g to about 150 meq/100 g. In some embodiments, the clays have a cation exchange capacity of from about 30 meq/100 g to about 100 meq/100 g.

In some embodiments, the collagen stimulating compositions and methods of making and using thereof optionally comprise a composite particle having an anionically charged clay electrostatically complexed with the cationically charged hair conditioning agents as disclosed herein.

Commercially available synthetic hectorites include those products sold under the trade names Laponite® RD, Laponite® RDS, Laponite® XLG, Laponite® XLS, Laponite® D, Laponite® DF, Laponite® DS, Laponite® S, and Laponite® JS (Southern Clay products, Texas, USA). Commercially available bentonites include those products sold under the trade names Gelwhite® GP, Gelwhite® H, Gelwhite® L, Mineral Colloid® BP, Mineral Colloid® MO, Gelwhite® MAS 100 (sc), Gelwhite® MAS 101, Gelwhite® MAS 102, Gelwhite® MAS 103, Bentolite® WH, Bentolite® L10, Bentolite® H, Bentolite® L, Permont® SX10A, Permont® SC20, and Permont® HN24 (Southern Clay Products, Texas, USA); Bentone® EW and Bentone® MA (Dow Corning); and Bentonite® USP BL 670 and Bentolite® H4430 (Whitaker, Clarke & Daniels). In some embodiments, the particles have a median particle size ranging from about 1 μm to about 100 μm. In some embodiments, the particles have a median particle size ranging from about 2 μm to about 50 μm. In some embodiments, the particles have a median particle size ranging from about 2 μm to about 20 μm. In some embodiments, the particles have a median particle size ranging from about 4 μm to about 10 μm. In some embodiments, the particles have a median particle size selected from: about 1 μm, about 1.1 μm, about 1.2 μm, about 1.3 μm, about 1.4 μm, about 1.5 μm, about 1.6 μm, about 1.7 μm, about 1.8 μm, about 1.9 μm, about 2.0 μm, about 2.1 μm, about 2.2 μm, about 2.3 μm, about 2.4 μm, about 2.5 μm, about 2.6 μm, about 2.7 μm, about 2.8 μm, about 2.9 μm, about 3.0 μm, about 3.1 μm, about 3.2 μm, about 3.3 μm, about 3.4 μm, about 3.5 μm, about 3.6 μm, about 3.7 μm, about 3.8 μm, about 3.9 μm, about 4.0 μm, about 4.1 μm, about 4.2 μm, about 4.3 μm, about 4.4 μm, about 4.5 μm, about 4.6 μm, about 4.7 μm, about 4.8 μm, about 4.9 μm, about 5.0 μm, about 5.1 μm, about 5.2 μm, about 5.3 μm, about 5.4 μm, about 5.5 μm, about 5.6 μm, about 5.7 μm, about 5.8 μm, about 5.9 μm, about 6.0 μm, about 6.1 μm, about 6.2 μm, about 6.3 μm, about 6.4 μm, about 6.5 μm, about 6.6 μm, about 6.7 μm, about 6.8 μm, about 6.9 μm, about 7.0 μm, about 7.1 μm, about 7.2 μm, about 7.3 μm, about 7.4 μm, about 7.5 μm, about 7.6 μm, about 7.7 μm, about 7.8 μm, about 7.9 μm, about 8.0 μm, about 8.1 μm, about 8.2 μm, about 8.3 μm, about 8.4 μm, about 8.5 μm, about 8.6 μm, about 8.7 μm, about 8.8 μm, about 8.9 μm, about 9.0 μm, about 9.1 μm, about 9.2 μm, about 9.3 μm, about 9.4 μm, about 9.5 μm, about 9.6 μm, about 9.7 μm, about 9.8 μm, about 9.9 μm, and about 10.0 μm.

In some embodiments, the weight ratio of the cationically charged hair conditioning agent to the clay is from 0.05:1 to 20:1. In some embodiments, the weight ratio of the cationically charged hair conditioning agent to the clay is from 0.1:1 to 10:1. In some embodiments, the weight ratio of the cationically charged hair conditioning agent to the clay is from 0.2:1 to 5:1. In some embodiments, the weight ratio of the cationically charged hair conditioning agent to the clay is selected from 0.05:1, 0.1:1, 0.2:1, 0.5:1, 0.75:1, 1:1, 1.5:1, 2:1, 2.5:1, 3:1, 3.5:1, 4.0:1, 4.5:1, 5.0:1, 5.5:1, 6.0:1, 6.5:1, 7.0:1, 7.5:1, 8.0:1, 8.5:1, 9.0:1, 9.5:1, 10.0:1, 10.5:1, 11.0:1, 11.5:1, 12.0:1, 12.5:1, 13.0:1, 13.5:1, 14.0:1, 14.5:1, 15.0:1, 15.5:1, 16.0:1, 16.5:1, 17.0:1, 17.5:1, 18.0:1, 18.5:1, 19.0:1, 19.5:1, ND 20.0:1.

In some embodiments, the particle is present in the collagen stimulating compositions and methods of making and using thereof at a weight percent ranging from about 0.01 wt. % to about 10.0 wt. % by the total weight of the silk collagen boosting composition. In some embodiments, the particle is present in the collagen stimulating compositions and methods of making and using thereof at a weight percent ranging from about 0.1 wt. % to about 10.0 wt. % by the total weight of the silk collagen boosting composition. In some embodiments, the particle is present in the composition at a weight percent ranging from about 0.1 wt. % to about 2.0 wt. % by the total weight of the silk collagen boosting composition. In some embodiments, the particle is present in the composition at a weight percent ranging from about 1.0 wt. % to about 9.0 wt. % by the total weight of the silk collagen boosting composition. In some embodiments, the particle is present in the composition at a weight percent ranging from about 1.0 wt. % to about 5.0 wt. % by the total weight of the silk collagen boosting composition. In some embodiments, the particle is present in the composition at a weight percent selected from: about 0.01 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5.0 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6.0 wt. %, about 6.1 wt. %, about 6.2 wt. %, about 6.3 wt. %, about 6.4 wt. %, about 6.5 wt. %, about 6.6 wt. %, about 6.7 wt. %, about 6.8 wt. %, about 6.9 wt. %, about 7.0 wt. %, about 7.1 wt. %, about 7.2 wt. %, about 7.3 wt. %, about 7.4 wt. %, about 7.5 wt. %, about 7.6 wt. %, about 7.7 wt. %, about 7.8 wt. %, about 7.9 wt. %, about 8.0 wt. %, about 8.1 wt. %, about 8.2 wt. %, about 8.3 wt. %, about 8.4 wt. %, about 8.5 wt. %, about 8.6 wt. %, about 8.7 wt. %, about 8.8 wt. %, about 8.9 wt. %, about 9.0 wt. %, about 9.1 wt. %, about 9.2 wt. %, about 9.3 wt. %, about 9.4 wt. %, about 9.5 wt. %, about 9.6 wt. %, about 9.7 wt. %, about 9.8 wt. %, about 9.9 wt. %, and about 10.0 wt. % by the total weight of the composition.

In some embodiments, the collagen stimulating compositions and methods of making and using thereof optionally comprise a colloidal stabilizer to maintain particle dispersive stability, particularly of larger sized particles. Suitable colloidal stabilizer is selected from the group consisting of propylene oxide-ethylene oxide copolymers or ethyleneoxide-propylenoxide graphted polyethylenimines, polyoxyethylene (20-80 units POE) isooctylphenyl ether, fatty alcohol ethoxylates, polyethoxylated polyterephthalate block co-polymers containing polyvinylpyrrolidone, copolymers containing vinylpyrolidone repeating units, and combinations thereof.

In some embodiments, collagen stimulating compositions and methods of making and using thereof comprises an emulsion as the dermatologically acceptable carrier. In some embodiments, the dermatologically acceptable carrier exists as a conventional emulsion. In some embodiments, the dermatologically acceptable carrier exits as a microemulsion. In some embodiments, the dermatologically acceptable carrier exits as a water-in-oil emulsion. In some embodiments, the dermatologically acceptable carrier exits as an oil-in-water emulsion. In some embodiments, the dermatologically acceptable carrier exits as a nano-emulsion. In some embodiments, the dermatologically acceptable carrier exits as a water-in-silicone oil emulsion. In some embodiments, the dermatologically acceptable carrier exits as a silicone oil-in-water emulsion.

As used herein, the conventional emulsions have one continuous phase and one disperse phase, which is present as very small spheres stabilized by coating with surfactants. Depending on the nature of the continuous phase, the emulsions are described as oil-in-water or water-in-oil. These emulsions are kinetically stable in the ideal case, i.e. they are retained even for a prolonged period, but not indefinitely. During temperature fluctuations in particular, they may have a tendency toward phase separation as a result of sedimentation, creaming, thickening or flocculation.

As used herein, the microemulsions are thermodynamically stable, isotropic, fluid, optically clear single liquid phase containing a ternary system having three ingredients of an oily component, an aqueous component and a surfactant. Microemulsions arise when a surfactant, or more frequently a mixture of a surfactant and a cosurfactant, reduces the oil/water interfacial tension to extremely low values, often in the range 10³ to 10⁹, preferably 10⁴ to 10⁶ N/m, such that the two insoluble phases remain dispersed by themselves in a homogeneous manner as a result of the thermal agitation. Microemulsions often have bicontinuous structures with equilibrium regions, so-called subphases in the order of magnitude from 100 to 1000 Angstroms. The microemulsion refers to either one state of an O/W (oil-in-water) type microemulsion in which oil is solubilized by micelles, or a bicontinuous microemulsion in which the number of associations of surfactant molecules are rendered infinite so that both the aqueous phase and oil phase have a continuous structure.

For properties, the microemulsion appears transparent or translucent and may exist as a solution in a monophasic state in which all the formulated ingredients and components are uniformly dissolved therein.

Regardless of manufacturing processes, microemulsions may take the same state if they have the same formulation components and prepared at the same temperature. Therefore, the above-described three ingredients (oil, water and surfactant) and the remaining ingredients may be added and mixed in any orders as appropriate and may be agitated using mechanical forces at any power to consequently yield a microemulsion having substantially the same state (in appearance, viscosity, feeling of use, etc.).

Bicontinuous microemulsions comprise two phases, a water phase and an oil phase, in the form of extended adjoining and intertwined domains at whose interface stabilizing interface-active surfactants are concentrated in a monomolecular layer.

Bicontinuous micro emulsions form very readily, usually spontaneously due to the very low interfacial tension, when the individual components, water, oil and a suitable emulsifier system, are mixed. Since the domains have only very small extensions in the order of magnitude of nanometers in at least one dimension, the microemulsions appear visually transparent and are thermodynamically, i.e. indefinitely, stable in a certain temperature range depending on the emulsifier system used.

As used herein, the term nanoemulsions refer to emulsions presenting transparent or translucent appearances due to their nano particle sizes, e.g. less than 1000 nm.

Emulsifiers (e.g., surfactants) are substances which reduce the interfacial tension between liquid phases which are not miscible with one another, a polar phase, often water and a nonpolar, organic phase, and thus increase their mutual solubility. Surfactants have a characteristic structure feature of at least one hydrophilic and one hydrophobic structural unit. This structure feature is also referred to as amphiphilic.

Anionic, cationic, amphoteric and nonionic surfactants have conventionally been used as emulsifiers for production of emulsified cosmetic materials by emulsification of water and oily substances. However, since synthetic surfactants have been implicated in the destruction of skin surface tissue and constituting a cause of liver damage when entering the body, numerous naturally-derived protein-based emulsifiers including natural protein based emulsifiers have been employed because of their high safety.

Although emulsified cosmetic materials obtained using protein-based emulsifiers generally have a soft, moist feel during use, it is often the case finished products impart a crumbling feel and lack spreadability. The important factors for emulsifiers used in cosmetic products include not only safety and emulsifying power, but also feel during use. The disclosure provides the use of silk fibroin protein fragments as emulsifier (thereafter silk emulsifier) to stabilize the emulsion carrier for the collagen boosting composition disclosed herein.

In an embodiment, the collagen stimulating compositions and methods of making and using thereof comprises an emulsion as carrier having a silk emulsifier in the emulsifier system. Silk fibroin is an amphiphilic polymer with large hydrophobic domains occupying the major component of the polymer, which has a high molecular weight. The hydrophobic regions are interrupted by small hydrophilic spacers, and the N- and C-termini of the chains are also highly hydrophilic. The hydrophobic domains of the H-chain contain a repetitive hexapeptide sequence of Gly-Ala-Gly-Ala-Gly-Ser and repeats of Gly-Ala/Ser/Tyr dipeptides, which can form stable anti-parallel-sheet crystallites. The amino acid sequence of the L-chain is non-repetitive, so the L-chain is more hydrophilic and relatively elastic. The hydrophilic (Tyr, Ser) and hydrophobic (Gly, Ala) chain segments in silk fibroin molecules are arranged alternatively such that allows self-assembling of silk fibroin molecules.

In some embodiments, the emulsifier system comprises a silk emulsifier and a small molecule having high HLB value. The composition of hydrophobic repeating groups is one penta-peptide -Gly-Ala-Gly-Ala-Gly- for each hydrophilic -Ser-, the hydrophilic-hydrophobic balance (HLB) for the silk fibroin protein can be modified to a range from 7.95-16.74 in a hydrophilic environment created by the addition of a hydrophilic molecule having high HLB value (i.e. >10). This range of HLB value of the silk fibroin protein fragments allows the preparation of a wide range of emulsions from O/W type emulsions to W/O type emulsions. In some embodiments, the hydrophilic molecule having high HLB value is selected from the group consisting of glycerol HLB 11.28, butantetraol HLB 12.7, xylitol HLB 14.13, D-sorbitol HLB 15.55, inositol HLB 16.74, polysaccharide including hyaluronic acid, hyaluronate, carrageenan, pullulan, alginic acid, alginate, microbial exopolysaccharides, glucosamine, chondroitin sulfate, glycosaminoglycans, glucomannan, and combination thereof. In some embodiments, the emulsifier system comprises the silk emulsifier and glycerol.

In some embodiments, the silk emulsifier and hydrophilic molecule having high HLB value are incorporated in the emulsion carrier at a weight ratio of silk emulsifier to the hydrophilic molecule of 1:1 to 1:10. In some embodiments, the silk emulsifier and hydrophilic molecule having high HLB value are incorporated in the emulsion carrier at a weight ratio of silk emulsifier to the hydrophilic molecule selected from: 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3.0, 1:3.1, 1:3.2, 1:3.3, 1:3.4, 1:3.5, 1:3.6, 1:3.7, 1:3.8, 1:3.9, 1:4, 1:4.1, 1:4.2, 1:4.3, 1:4.4, 1:4.5, 1:4.6, 1:4.7, 1:4.8, 1:4.9, 1:5.0, 1:5.1, 1:5.2, 1:5.3, 1:5.4, 1:5.5, 1:5.6, 1:5.7, 1:5.8, 1:5.9, 1:6, 1:6.1, 1:6.2, 1:6.3, 1:6.4, 1:6.5, 1:6.6, 1:6.7, 1:6.8, 1:6.9, 1:7, 1:8, 1:9 and 1:10. In some embodiments, the silk emulsifier and hydrophilic molecule having high HLB value are incorporated in the emulsion carrier at a weight ratio of silk emulsifier to the hydrophilic molecule of 1:1. In some embodiments, the emulsifier system comprises the silk emulsifier and glycerol at a weight ratio of silk emulsifier to glycerol of 1:1 to 1:3. In some embodiments, the emulsifier system comprises the silk emulsifier and glycerol at a weight ratio of silk emulsifier to glycerol selected from: 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3.0.

In an embodiment, this disclosure provides an aqueous solution of silk fibroin protein fragments or the aqueous gel of silk fibroin protein based fragments as described above as emulsifier (hereafter as silk emulsifier) for the emulsion carrier. The aqueous solution of silk fibroin protein fragments or the aqueous gel of silk fibroin protein fragments as described above may be admixed with an oily component to achieve uniform emulsification between the water in the aqueous solution or aqueous gel of the silk fibroin protein fragments and the oily component.

In some embodiments, the silk fibroin protein fragments used as emulsifier has a weight average molecular weight of greater than about 5 kDa. In some embodiments, the silk fibroin protein used as emulsifier has a weight average molecular weight selected from about 5 kDa to about 350 kDa. In some embodiments, the silk fibroin protein used as emulsifier has a weight average molecular weight selected from between about 20 kDa to about 80 kDa. In some embodiments, the silk fibroin protein used as emulsifier has a weight average molecular weight selected from between about 40 kDa to about 60 kDa. In other embodiments, any silk fibroin fragments described herein can be used as emulsifiers.

In some embodiments, the amount of the silk emulsifier presented in the emulsion carrier ranges from about 0.1 wt. % to about 15.0 wt. % by the total weight of the emulsion carrier. In some embodiments, the amount of the silk emulsifier presented in the emulsion carrier ranges from about 0.75 wt. % to about 10.0 wt. % by the total weight of the emulsion carrier. In some embodiments, the amount of the silk emulsifier presented in the emulsion carrier is selected from the group consisting of about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.25 wt. %, about 1.50 wt. %, about 1.75 wt. %, about 2.0 wt. %, about 2.25 wt. %, about 2.5 wt. %, about 2.75 wt. %, about 3.0 wt. %, about 3.25 wt. %, about 3.5 wt. %, about 3.75 wt. %, about 4.0 wt. %, about 4.25 wt. %, about 4.5 wt. %, about 4.75 wt. %, about 5.0 wt. %, about 5.25 wt. %, about 5.5 wt. %, about 5.75 wt. %, about 6.0 wt. %, about 6.25 wt. %, about 7.5 wt. %, about 7.75 wt. %, about 8.0 wt. %, about 8.25 wt. %, about 8.5 wt. %, about 8.75 wt. %, about 9.0 wt. %, about 9.25 wt. %, about 9.5 wt. %, about 9.75 wt. %, about 10.0 wt. %, about 10.25 wt. %, about 10.5 wt. %, about 10.75 wt. %, about 11.0 wt. %, about 11.25 wt. %, about 11.5 wt. %, about 11.75 wt. %, about 12.0 wt. %, about 12.25 wt. %, about 12.50 wt. %, about 12.75 wt. %, about 13.0 wt. %, about 13.25 wt. %, about 13.50 wt. %, about 13.75 wt. %, about 14.0 wt. %, about 14.25 wt. %, about 14.50 wt. %, about 14.75 wt. %, and about 15.0 wt. %.

Silk protein in the aqueous solution tends to fibrillate more readily by shear of vibration or stirring if it has a higher molecular weight. The fibrillated protein consists of water-insoluble masses causes reduction of pleasant feel during use of the cosmetic materials.

In some embodiments, the silk fibroin protein fragments are blended with hydrophilic substance with high HLB value to enhance the hydrophilic environment and such hydrophilic substance includes glycerol, butantetraol, xylitol, D-sorbitol, inositol polyethylene glycol, polyethylene oxide, polylactic acid, cellulose, chitin and polyvinyl alcohol to prevent silk fibroin solution from gelation. It is important to prevent fibroin transformation from random coils to β-sheet structure (fibrillate).

In some embodiments, a sucrose fatty ester based emulsifier having HLB value>10 is added to the silk fibroin protein as emulsion stabilizer to enhance silk fibroin protein emulsification efficiency.

In some embodiments, the emulsifying system for the collagen stimulating compositions and methods of making and using thereof may include a sucrose fatty ester based emulsifier and an aqueous solution of silk fibroin protein or the aqueous gel of silk fibroin protein.

In some embodiments, an aqueous solution or an aqueous gel containing silk fibroin protein fragments may be used as co-emulsifier for the collagen stimulating compositions, wherein the aqueous solution or gel of silk protein is obtained by dissolving unscoured, partially scoured or scoured spun silkworm fibers (cocoon filaments) with a neutral salt (e.g. lithium bromide). In some embodiments, the sucrose fatty ester is sucrose palmitate and sucrose laurate ester. In some embodiments, silk proteins may be employed as surfactants for the collagen stimulating compositions with enhanced emulsifying efficiency. In some embodiments, phospholipids (e.g., lecithin) may be used to complex with silk fibroin protein fragments derived co-emulsifiers to increase their emulsifying power (efficiency of surfactant).

In some embodiments, the collagen stimulating compositions containing microemulsion obtained using silk fibroin protein fragments-based emulsifier generally have good spreadability, a soft, and moist feel during use. In some embodiments, the emulsion carrier for the collagen stimulating compositions and methods of making and using thereof may further comprise one or more ionic surfactants as co-emulsifiers.

An ionic surfactant is a surfactant that is ionized to have an electric charge in an aqueous solution; depending on the type of the electric charge, it is classified into ampholytic surfactants, cationic surfactants, or anionic surfactants. When an anionic surfactant and an ampholytic surfactant, or an anionic surfactant and a cationic surfactant, are mixed in an aqueous solution, the interfacial tension against oil decreases.

An ampholytic surfactant has at least one cationic functional group and one anionic functional group, is cationic when the solution is acidic and anionic when the solution is alkaline, and assumes characteristics similar to a nonionic surfactant around the isoelectric point.

Ampholytic surfactants are classified, based on the type of the anionic group, into the carboxylic acid type, the sulfuric ester type, the sulfonic acid type, and the phosphoric ester type. For the present invention, the carboxylic acid type, the sulfuric ester type, and the sulfonic acid type are preferable. The carboxylic acid type is further classified into the amino acid type and the betaine type. Particularly preferable is the betaine type.

Specific examples include: imidazoline type ampholytic surfactants (for example, 2-undecyl-1-hydroxyethyl-1-carboxymethyl-4,5-dihydro-2-imidazolium sodium salt and 1-[2-(carboxymethoxy)ethyl]-1-(carboxymethyl)-4,5-dihydro-2-norcocoalkylimidazolium hydroxide disodium salt); and betaine type surfactants (for example, 2-heptadecyl-N-carboxymethyl-N-hydroxyethyl imidazolinium betaine, lauryldimethylarninoacetic acid betaine, alkyl betaine, amide betaine, and sulfobetaine).

Examples of the cationic surfactant include quaternary ammonium salts such as cetyltrimethylammonium chloride, stearyltrimethylammonium chloride, benenyltrimethylammonium chloride, behenyldimethylhydroxyethylammonium chloride, stearyldimethylbenzylammonium chloride, and cetyltrimethylammonium methylsulfate. Other examples include amide amine compounds such as stearic diethylaminoethylamide, stearic dimethylaminoethylamide, palmitic diethylaminoethylamide, palmitic dimethylaminoethylamide, myristic diethylaminoethylamide, myristic dimethylaminoethylamide, behenic diethylaminoethylamide, behenic dimethylaminoethylamide, stearic diethylaminopropylamide, stearic dimethylaminopropylamide, palmitic diethylaminopropylamide, palmitic dimethylaminopropylamide, myristic diethylaminopropylamide, myristic dimethylaminopropylamide, behenic diethylaminopropylamide, and behenic dimethylaminopropylamide.

In some embodiments, the emulsifier system for the collagen stimulating compositions and methods of making and using thereof may further comprise one or more anionic surfactants. Anionic surfactants are classified into the carboxylate type such as fatty acid soaps, N-acyl glutamates, and alkyl ether acetates, the sulfonic acid type such as α-olefin sulfonates, alkane sulfonates, and alkylbenzene sulfonates, the sulfuric ester type such as higher alcohol sulfuric ester salts, and phosphoric ester salts. Preferable are the carboxylate type, the sulfonic acid type, and the sulfuric ester salt type; particularly preferable is the sulfuric ester salt type.

In some embodiments, the anionic surfactant for the collagen stimulating compositions and methods of making and using thereof is selected from the group consisting of higher alkyl sulfuric acid ester salts (for example, sodium lauryl sulfate and potassium lauryl sulfate); alkyl ether sulfuric acid ester salts (e.g., POE-triethanolamine lauryl sulfate and sodium POE-lauryl sulfate); N-acyl sarcosinic acids (e.g., sodium lauroyl sarcosinate); higher fatty acid amide sulfonic acid salts (e.g., sodium N-myristoyl N-methyl taurate, Sodium N-cocoyl-N-methyl taurate, and Sodium jauroylmethyl taurate); phosphoric ester salts (e.g., sodium POE-oleyl ether phosphate and POE stearyl ether phosphoric acid); sulfosuccinates (e.g., sodium di-2-ethylhexylsulfosuccinate, sodium monolauroyl monoethanol amide polyoxyethylene sulfosuccinate, and sodium lauryl polypropylene glycol sulfosuccinate); alkyl benzene sulfonates (e.g., sodium linear dodecyl benzene sulfonate, triethanolamine linear dodecyl benzene sulfonate, and linear dodecyl benzene sulfonic acid); higher fatty acid ester sulfates (e.g., hydrogenated coconut oil aliphatic acid glyceryl sodium sulfate); N-acyl glutamates (e.g., mono sodium N-lauroylglutamate, disodium N-stearoylglutamate, and sodium N-myristoyl-L-glutamate); sulfated oils (e.g., turkey red oil); POE-alkyl ether carboxylic acid; POE-alkyl aryl ether carboxylate; α-olefin sulfonate; higher fatty acid ester sulfonates; sec-alcohol sulfates; higher fatty acid alkyl amide sulfates; sodium lauroyl monoethanolamine succinates; ditriethanolamine N-palmitoylaspartate; and sodium caseinate.

In some embodiments, the emulsifier system for the collagen stimulating compositions and methods of making and using thereof may further comprise one or more nonionic surfactants as co-emulsifiers. The nonionic surfactant preferably has an HLB value of 8.9-14. It is generally known that the solubility into water and the solubility into oil balance when the HLB is 7. That is, a surfactant preferable for the present invention would have medium solubility in oil/water.

The nonionic surfactants may include: (1) polyethylene oxide extended sorbitan monoalkylates (e.g., polysorbates); (2) polyalkoxylated alkanols; (3) polyalkoxylated alkylphenols include polyethoxylated octyl or nonyl phenols having HLB values of at least about 14, which are commercially available under the trade designations ICONOL® and TRITON®; (4) polaxamers. Surfactants based on block copolymers of ethylene oxide (EO) and propylene oxide (PO) may also be effective. Both EO-PO-EO blocks and PO-EO-PO blocks are expected to work well as long as the HLB is at least about 14, and preferably at least about 16. Such surfactants are commercially available under the trade designations PLURONIC® and TETRONIC® from BASF; (5) polyalkoxylated esters: polyalkoxylated glycols such as ethylene glycol, propylene glycol, glycerol, and the like may be partially or completely esterified, i.e. one or more alcohols may be esterified, with a (C8 to C22) alkyl carboxylic acid. Such polyethoxylated esters having an HLB of at least about 14, and preferably at least about 16, may be suitable for use in compositions of the present invention; (6) alkyl polyglucosides. This includes glucopon 425, which has a (C8 to C16) alkyl chain length; (7) sucrose fatty acid ester having high HLB value (8-18): sucrose cocoate, sucrose dilaurate, sucrose distearate, sucrose hexaerucate, sucrose hexaoleate/hexapalmitate/hexstearate, sucrose hexapalmitate, sucrose laurate, sucrose myristate, sucrose oleate, sucrose palmitate, sucrose pentaerucate, sucrose polybehenate, sucrose polycottonseedate, sucrose polylaurate, sucrose polylinoleate, sucrose polyoleate, sucrose polypalmate, sucrose polysoyate, sucrose polystearate, sucrose ricinoleate, sucrose stearate, sucrose tetraisostearate, sucrose trilaurate.

In some embodiments, the emulsifier system comprises a lipophilic nonionic surfactants selected from the group consisting of sorbitan fatty acid esters (e.g., sorbitan mono oleate monooleate, sorbitan mono isostearate monoisostearate, sorbitan mono laurate monolaurate, sorbitan mono palmitate monopalmitate, sorbitan mono stearate monostearate, sorbitan sesquioleate, sorbitan trioleate, diglyceryl sorbitan penta-2-ethylhexylate, diglyceryl sorbitan tetra-2-ethylhexylate); glyceryl and polyglyceryl aliphatic acids (e.g., mono cottonseed oil fatty acid glycerine, glyceryl monoerucate, glyceryl sesquioleate, glyceryl monostearate, α,α′-glyceryl oleate pyroglutamate, monostearate glyceryl malic acid); propylene glycol fatty acid esters (e.g., propylene glycol monostearate); hydrogenated castor oil derivatives; glyceryl alkylethers, and combination thereof.

In some embodiments, the emulsifier system comprises a hydrophilic nonionic surfactants selected from the group consisting of POE-sorbitan fatty acid esters (e.g., POE-sorbitan monooleate, POE-sorbitan monostearate, POE-sorbitan monooleate, and POE-sorbitan tetraoleate); POE sorbitol fatty acid esters (e.g., POE sorbitol monolaurate, POE-sorbitol monooleate, POE-sorbitolpentaoleate, and POE-sorbitol monostearate); POE-glyceryl fatty acid esters (e.g., POE-monooleates such as POE-glyceryl monostearate, POE-glyceryl monoisostearate, and POE glycerin glyceryl triisostearate); POE-fatty acid esters (e.g, POE-distearate, POE-monodioleate, and ethylene glycol distearate); POE-alkylethers (e.g., POE-lauryl ether, POE-oleyl ether, POE-stearyl ether, POE-behenyl ether, POE 2-octyl dodecyl ether, and POE-cholestanol ether); pluaronics (e.g., pluaronic); POE-POP-alkylethers (e.g, POE-POP-cetyl ether, POE-POP2-decyl tetradecyl ether, POE-POP-monobutyl ether, POE-POP-lanolin hydrate, and POE-POP glycerin glyceryl ether); tetra POE-tetra POP-ethylenediamino condensates (e.g., tetronic); POE-castor oil hydrogenated castor oil derivatives (e.g., POE-castor oil, POE-hydrogenated castor oil, POE-hydrogenated castor oil monoisostearate, POE-hydrogenated castor oil triisostearate, POE-hydrogenated castor oil monopyroglutamic monoisostearic diester, and POE-hydrogenated castor oil maleic acid); POE-beeswax-lanolin derivatives (e.g., POE-sorbitol beeswax); alkanol amides (e.g., palm oil fatty acid diethanol amide, laurate monoethanolamide, and fatty acid isopropanol amide); POE-propylene glycol fatty acid esters; POE-alkylamines; POE-fatty acid amides; sucrose fatty acid esters; alkyl ethoxydimethylamine oxides; and trioleyl phosphoric acid.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof, carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof, polyoxyethylated vitamins and derivatives thereof; polyoxyethylene-polyoxypropylene block copolymers; and mixtures thereof, polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In some embodiments, the emulsifier system comprises mono-glycerol derivatives and/or diglycerol derivatives. Specific examples include: monoglycerol derivatives such as monoglycerol monooctanoate, monooctyl monoglyceryl ether, monoglycerol monononanoate, monononyl monoglyceryl ether, monoglycerol monodecanoate, monodecyl monoglyceryl ether, monoglycerol monoundecylenate, monoundecylenyl glyceryl ether, monoglycerol monododecanoate, monododecyl monoglyceryl ether, monoglycerol monotetradecanoate, monoglycerol monohexadecanoate, monoglycerol monooleate, and monoglycerol monoisostearate, as well as diglycerol derivatives such as diglycerol monooctanoate, monooctyl diglyceryl ether, diglycerol monononanoate, monononyl diglyceryl ether, diglycerol monodecanoate, monodecyl diglyceryl ether, diglycerol monoundecylenate, monoundecylenyl glyceryl ether, diglycerol monododecanoate, monododecyl diglyceryl ether, diglycerol monotetradecanoate, diglycerol monohexadecanoate, diglycerol monooleate, and diglycerol monoisostearate.

In some embodiments, the emulsifier system comprises the silk emulsifier and one or more of sucrose laurate, and sucrose palmitate. In some embodiments, the emulsifier system comprises the silk emulsifier and sucrose laurate. In some embodiments, the emulsifier system comprises the silk emulsifier and sucrose palmitate. In some embodiments, the emulsifier system comprises the silk emulsifier, sucrose laurate, and sucrose palmitate, wherein sucrose laurate, and sucrose palmitate in the emulsion carrier has a weight ratio of sucrose laurate to sucrose palmitate ranging from 1:1 to 1:3. In some embodiments, the emulsifier system comprises the silk emulsifier, sucrose laurate, and sucrose palmitate, wherein sucrose laurate, and sucrose palmitate in the emulsion carrier has a weight ratio of sucrose laurate to sucrose palmitate selected from: 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, and 1:3.0.

In some embodiments, the emulsifier system comprises the silk emulsifier, sucrose laurate, and sucrose palmitate, wherein sucrose laurate, and sucrose palmitate in the emulsion carrier has a weight ratio of sucrose laurate to sucrose palmitate selected from: 1:1, 1:1.1, 1:1.2 and 1:1.3. In some embodiments, the emulsifier system comprises the silk emulsifier, sucrose laurate, and sucrose palmitate, wherein sucrose laurate, and sucrose palmitate in the emulsion carrier has a weight ratio of sucrose laurate to sucrose palmitate of 1:1.

In some embodiments, the emulsifier system comprises the silk emulsifier, glycerol, sucrose laurate, and sucrose palmitate, wherein sucrose laurate and sucrose palmitate in the emulsion carrier has a weight ratio of sucrose laurate to sucrose palmitate selected from: 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, and 1:3.0, wherein the silk emulsifier and the glycerol in the emulsion carrier has a weight ratio of silk emulsifier to glycerol selected from: 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, and 1:3.0.

In some embodiments, the emulsifier system comprises the silk emulsifier, glycerol, sucrose laurate, and sucrose palmitate, wherein sucrose laurate and sucrose palmitate in the emulsion carrier has a weight ratio of sucrose laurate to sucrose palmitate selected from: 1:1, 1:1.1, 1:1.2, and 1:1.3, wherein the silk emulsifier and the glycerol in the emulsion carrier has a weight ratio of silk emulsifier to glycerol selected from: 1:1, 1:2, and 1:3.0.

In some embodiments, the emulsifier system is incorporated in the emulsion carrier at a weight percent ranging from 0.1 wt. % to 5.0 wt. % by the total weight of the collagen boosting composition. In some embodiments, the emulsifier system is incorporated in the emulsion carrier at a weight percent ranging from 0.1 wt. % to 3.0 wt. % by the total weight of the collagen boosting composition. In some embodiments, the emulsifier system is incorporated in the emulsion carrier at a weight percent ranging from 0.1 wt. % to 2.0 wt. % by the total weight of the collagen boosting composition.

In some embodiments, the emulsion carrier comprises an oil phase emulsified with the emulsifier system containing the silk emulsifier as described above. The fatty materials may be useful for forming the oil phase. The fatty material is selected from the group consisting of hydrocarbon oils, silicon oil, higher fatty acids, higher alcohols, synthetic ester oils, silicone oils, liquid oils/fats, solid oils/fats, waxes, and combination thereof.

In an embodiment, the fatty material optionally comprises a wax. The wax is selected from the group consisting of polyethylene wax, polypropylene wax, beeswax, candelilla wax, paraffin wax, ozokerite, microcrystalline waxes, carnauba wax, cotton wax, esparto wax, carnauba wax, bayberry wax, tree wax, whale wax, montan wax, bran wax, lanolin, kapok wax, lanolin acetate, liquid lanolin, sugar cane wax, lanolin fatty acid isopropyl ester, hexyl laurate, reduced lanolin, jojoba wax, hard lanolin, shellac wax, POE lanolin alcohol ether, POE lanolin alcohol acetate, POE cholesterol ether, lanolin fatty acid polyethylene glycol, POE hydrogenated lanolin alcohol ether, and combination thereof.

In an embodiment, the fatty material optionally comprises an ester oil. The ester oil is selected from the group consisting of cholesteryl isostearate, isopropyl palmitate, isopropyl myristate, neopentylglycol dicaprate, isopropyl isostearate, octadecyl myristate, cetyl 2-ethylhexanoate, cetearyl isononanoate, cetearyl octanoate, isononyl isononanoate, isotridecyl isononanoate, glyceryl tri-2-ethylhexanoate, glyceryl tri(caprylatelcaprate), diethylene glycol monoethyl ether oleate, dicaprylyl ether, caprylic acid/capric acid propylene glycol diester, and combination thereof.

In an embodiment, the fatty material optionally comprises a glyceride fatty ester. As used herein, the term “glyceride fatty esters” refers to the mono-, di-, and tri-esters formed between glycerol and long chain carboxylic acids such as C₆-C₃₀ carboxylic acids. The carboxylic acids may be saturated or unsaturated or contain hydrophilic groups such as hydroxyl. Preferred glyceride fatty esters are derived from carboxylic acids of carbon chain length ranging from C₁₀ to C₂₄, preferably C₁₀ to C₂₂ most preferably C₁₂ to C₂₀.

In an embodiment, the fatty material optionally comprises synthetic ester oils. In some embodiments, the synthetic ester oil is selected from the group consisting of isopropyl myristate, cetyl octanoate, octyldodecyl myristate, isopropyl palmitate, butyl stearate, hexyl laurate, myristyl myristate, decyl oleate, hexyldecyl dimethyloctanoate, cetyl lactate, myristyl lactate, lanolin acetate, isocetyl stearate, isocetyl isostearate, cholesteryl 12-hydroxystearate, ethylene glycol di-2-ethylhexylate, dipentaerythritol fatty acid ester, N-alkyl glycol monoisostearate, neopentyl glycol dicaprate, diisostearyl malate, glyceryl di-2-heptylundecanoate, trimethylolpropane tri-2-ethylhexylate, trimethylolpropane triisostearate, pentaneerythritol tetra-2-ethylhexylate, glyceryl tri-2-ethylhexylate, trimethylolpropane triisostearate, cetyl 2-ethylhexanoate, 2-ethylhexyl palmitate, glyceryl trimyristate, tri-2-heptylundecanoic glyceride, castor oil fatty acid methyl ester, oleyl oleate, cetostearyl alcohol, acetoglyceride, 2-heptylundecyl palmitate, diisopropyl adipate, N-lauroyl-L-glutamic acid-2-octyldodecyl ester, di-2-heptylundecyl adipate, ethyl laurate, di-2-ethylhexyl cebatate. 2-hexyldecyl myristate, 2-hexyldecyl palmitate, 2-hexyldecyl adipate, diisopropyl cebatate, 2-ethylhexyl succinate, ethyl acetate, butyl acetate, amyl acetate and triethyl |citrate, and combination thereof.

In an embodiment, the fatty material optionally comprises ether oil. In some embodiments, the ether oils are selected from the group consisting of alkyl-1,3-dimethylethyl ether, nonylphenyl ether, and combination thereof.

In an embodiment, the fatty material optionally comprises higher fatty acids. As used herein, the higher fatty acids have a carbon number ranging from 8 to 22. In some embodiments, the higher fatty acid is selected from the group consisting of lauric acid, myristic acid, palmitic acid, stearic acid, behenic acid, oleic acid, 12-hydroxystearic acid, undecylenic acid, tall oil, isostearic acid, linoleic acid, linolenic acid, eicosapentaenoic acid (EPA), docosahexaenoic acid (DHA), and combination thereof.

In an embodiment, the fatty material optionally comprises higher fatty alcohols. As used herein, the higher fatty alcohols have a carbon number ranging from 8 to 22. In some embodiments, the higher fatty acid is selected from the group consisting of straight chain alcohols (for example, lauryl alcohol, cetyl alcohol, stearyl alcohol, behenyl alcohol, myristyl alcohol, oleyl alcohol, and cetostearyl alcohol) and branched chain ethyl alcohols (for example, mono stearyl glyceryl ether (batyl alcohol), 2-decyltetradecynol, lanolin alcohol, cholesterol, phytosterol, hexyl dodecanol, isostearyl alcohol, and octyl dodecanol), and combination thereof.

In some embodiments, the fatty phase comprises liquid oils/fats. In some embodiments, the liquid oils/fats are selected from the group consisting of avocado oil, tsubaki oil, turtle oil, macademia nut oil, corn oil, mink oil, olive oil, rape seed oil, egg yolk oil, sesame seed oil, persic oil, wheat germ oil, sasanqua oil, castor oil, linseed oil, safflower oil, cotton seed oil, perilla oil, soybean oil, peanut oil, tea seed oil, kaya oil, rice bran oil, chinese wood oil, Japanese wood oil, jojoba oil, germ oil, triglycerol, glyceryl trioctanoate and glyceryl triisopalmitate, and combination thereof.

In some embodiments, the fatty phase comprises solid fats/oils. In some embodiments, the solid oils/fats are selected from the group consisting of cacao butter, coconut oil, horse tallow, hardened coconut oil, palm oil, beef tallow, sheep tallow, hardened beef tallow, palm kernel oil, pork tallow, beef bone tallow, Japanese core wax, hardened oil, neatsfoot tallow, Japanese wax and hydrogenated castor oil, and combination thereof.

In some embodiments, the fatty phase comprises vegetable oils. In some embodiments, the vegetable oils are selected from the group consisting of buriti oil, soybean oil, olive oil, tea tree oil, rosemary oil, jojoba oil, coconut oil, sesame seed oil, sesame oil, palm oil, avocado oil, babassu oil, rice oil, almond oil, argon oil, sunflower oil, and combination thereof. In some embodiments, the vegetable oil is selected from the group consisting of coconut oil, sunflower oil and sesame oil. In some embodiments, the oily component is selected from cocoa butter, palm stearin, sunflower oil, soybean oil and coconut oil.

In some embodiments, the oil phase for the collagen stimulating compositions and methods of making and using thereof comprises lipid material. In some embodiments, the lipid materials are selected from the group consisting of ceramides, phospholipids (e.g., soy lecithin, egg lecithin), glycolipids, and combination thereof.

In some embodiments, the oil phase for the collagen stimulating compositions and methods of making and using thereof comprises hydrocarbon oil. As used herein, the hydrocarbon oils have average carbon chain length less than 20 carbon atoms. Suitable hydrocarbon oils include cyclic hydrocarbons, straight chain aliphatic hydrocarbons (saturated or unsaturated), and branched chain aliphatic hydrocarbons (saturated or unsaturated). Straight chain hydrocarbon oils will typically contain from about 6 to about 16 carbon atoms, preferably from about 8 up to about 14 carbon atoms. Branched chain hydrocarbon oils can and typically may contain higher numbers of carbon atoms, e.g. from about 6 up to about 20 carbon atoms, preferably from about 8 up to about 18 carbon atoms. Suitable hydrocarbon oils of the invention will generally have a viscosity at ambient temperature (25 to 30° C.) of from 0.0001 to 0.5 Pa·s, preferably from 0.001 to 0.05 Pa·s, more preferably from 0.001 to 0.02 Pa·s.

In some embodiments, the hydrogen carbon oils are selected from the group consisting of liquid petrolatum, squalane, pristane, paraffin, isoparaffin, ceresin, squalene, mineral oil, light mineral oil, blend of light mineral oil and heavy mineral oil, polyisobutene, hydrogenated polyisobutene, terpene oil and combination thereof.

In some embodiments, the hydrogen carbon oils light mineral oil. As used herein, mineral oils are clear oily liquids obtained from petroleum oil, from which waxes have been removed, and the more volatile fractions removed by distillation. The fraction distilling between 250° C. to 300° C. is termed mineral oil, and it consists of a mixture of hydrocarbons, in which the number of carbon atoms per hydrocarbon molecule generally ranges from C10 to C40. Mineral oil may be characterized in terms of its viscosity, where light mineral oil is relatively less viscous than heavy mineral oil, and these terms are defined more specifically in the U.S. Pharmacopoeia, 22nd revision, p. 899 (1990). A commercially available example of a suitable light mineral oil for use in the invention is Sirius® M40 (carbon chain length C0-C28 mainly C12-C20, viscosity 4.3×10 Pa·s), available from Silkolene. Other hydrocarbon oils that may be used in the invention include relatively lower molecular weight hydrocarbons including linear saturated hydrocarbons such a tetradecane, hexadecane, and octadecane, cyclic hydrocarbons such as dioctylcyclohexane (e.g. CETIOL® S from Henkel), branched chain hydrocarbons (e.g. ISOPAR® and ISOPAR® V from Exxon Corp.).

In some embodiments, the fatty material for the oil phase is selected from the group consisting of neopentyl glycol diheptanoate, propylene glycol dicaprylate, dioctyl adipate, coco-caprylate/caprate, diethylhexyl adipate, diisopropyl dimer dilinoleate, diisostearyl dimer dilinoleate, butyrospermum parkii (shea) butter, C12-C13 alkyl lactate, di-C12-C13 alkyl tartrate, tri-C12-C13 alkyl citrate, C12-C15 alkyl lactate, ppg dioctanoate, diethylene glycol dioctanoate, meadow foam oil, C12-15 alkyl oleate, tridecyl neopentanoate, cetearyl alcohol and polysorbate 60, C18-C26 triglycerides, cetearyl alcohol & cetearyl glucoside, acetylated lanolin, vp/eicosene copolymer, glyceryl hydroxystearate, C18-36 acid glycol ester, C18-36 triglycerides, glyceryl hydroxystearate and mixtures thereof. also suitable and preferred are cetyl alcohol & glyceryl stearate & PEG-75, stearate & ceteth-20 & steareth-20, lauryl glucoside & polyglyceryl-2 dipolyhydroxystearate, beheneth-25, polyamide-3 & pentaerythrityl tetra-di-t-butyl hydroxycinnamate, polyamide-4 and PEG-100 stearate, potassium cethylphosphate, stearic acid and hectorites.

In some embodiments, the fatty material for the oil phase is selected from the group consisting of liquid paraffin, liquid isoparaffin, neopentylglycol dicaprate, isopropyl isostearate, cetyl 2-ethylhesanoate, isononyl isononanoate, glyceryl tri(caprylatelcaprate), alky-1,3-dimethylbutyl ether, methyl polysiloxane having a molecular weight ranging from 100 to 500, decamethylcydopentasiloxane, octamethylcydotetrasiloxane, higher fatty acids having a carbon number ranging from 12 to 22, higher alcohols having a carbon number ranging from 12 to 22, ceramides, glycolipids, and terpene oil.

In some embodiments, the fatty material for the oil phase is selected from the group consisting of paraffin oil, glyceryl stearate, isopropyl myristate, diisopropyl adipate, cetylstearyl 2-ethylhexanoate, hydrogenated polyisobutene, Vaseline, caprylic/capric triglycerides, microcrystalline wax, lanolin and stearic acid, silicone oils and combination thereof.

In an embodiment, the fatty material for the oil phase is selected from the group consisting of vegetable oils including jojoba oil, olive oil, camella oil, avocado oil, cacao oil, sunflower oil, persic oil, palm oil, castor oil, buriti oil, medium chain triglycerides.

In an embodiment, the oily materials emulsifiable by the silk emulsifier is selected from the group consisting of a vegetable oil, isododecane, and isohexadecane, and one or more oily esters of fatty acids, wherein the vegetable oil is selected from jojoba oils and/or camellia oils, wherein said oily esters are selected from isononyl isononanoate and coco caprylate.

In some embodiments, the oil phase is present in the collagen stimulating compositions and methods of making and using thereof at a weight percent ranging from 1.0 wt. % to about 95 wt. % by the total weight of the collagen boosting composition. In some embodiments, the oil phase is present in the collagen boosting composition at a weight percent ranging from 45.0 wt. % to about 95 wt. % by the total weight of the collagen boosting composition. In some embodiments, the oil phase is present in the collagen boosting composition at a weight percent ranging from 45.0 wt. % to about 65.0 wt. % by the total weight of the collagen boosting composition. In some embodiments, the oil phase is present in the collagen boosting composition at a weight percent ranging from 5.0 wt. % to about 45 wt. % by the total weight of the collagen boosting composition. In some embodiments, the oil phase is present in the collagen boosting composition at a weight percent ranging from 5.0 wt. % to about 35 wt. % by the total weight of the collagen boosting composition. In some embodiments, the oil phase is present in the collagen boosting composition at a weight percent ranging from 10.0 wt. % to about 25 wt. % by the total weight of the collagen boosting composition.

In some embodiments, the oil phase is presented in the collagen stimulating compositions and methods of making and using thereof in a weight percent ranging from about 50.0 wt. % to 95.0 weight % by the total weight of the emulsion carrier. In some embodiments, the oil phase is presented in the collagen boosting composition in a weight percent ranging from about 5 wt. % to 45 weight % by the total weight of the emulsion carrier, because such a content allows the emulsion carrier to have a stability over a wider temperature range around the room temperatures and a good feeling.

In some embodiments, the aqueous phase for the emulsion carrier comprises water, an aqueous solution, a blend of alcohol and water, or a lyotropic liquid crystalline phase as aqueous carrier. Selection of the water contained in the collagen stimulating compositions and methods of making and using thereof of the present invention is not limited in particular; specific examples include purified water, ion-exchanged water, and tap water. In some embodiments, the aqueous further comprise one or more small molecule polyhydric alcohols selected from the group consisting of ethanediol, propanediol, glycerol, butanediol, butantetraol, xylitol, sorbitol, inositol, ethylene glycol, polyethylene glycol. In some embodiments, the aqueous phase further comprise one or more low alcohol solvent including methanol, ethanol, and isopropanol.

The blend ratio of water and polyhydric alcohol is determined appropriately based on emulsion formulation types.

In some embodiments, the emulsion comprises from about 50 wt. % to about 98 wt. % of the aqueous phase by the total weight of the composition. In some embodiments, the emulsion comprises from about 60 wt. % to about 90 wt. % of the aqueous phase by the total weight of the composition. In some embodiments, the amount of the aqueous phase in the emulsion is selected from: about 50.0 wt. %, about 51.0 wt. %, about 52.0 wt. %, about 53.0 wt. %, about 54.0 wt. %, about 55.0 wt. %, about 56.0 wt. %, about 57.0 wt. %, about 58.0 wt. %, about 59.0 wt. %, about 60.0 wt. %, about 61.0 wt. %, about 62.0 wt. %, about 63.0 wt. %, about 64.0 wt. %, about 65.0 wt. %, about 66.0 wt. %, about 67.0 wt. %, about 68.0 wt. %, about 69.0 wt. %, about 70.0 wt. %, about 71.0 wt. %, about 72.0 wt. %, about 73.0 wt. %, about 74.0 wt. %, about 75.0 wt. %, about 76.0 wt. %, about 77.0 wt. %, about 78.0 wt. %, about 79.0 wt. %, about 80.0 wt. %, about 81.0 wt. %, about 82.0 wt. %, about 83.0 wt. %, about 84.0 wt. %, about 85.0 wt. %, about 86.0 wt. %, about 87.0 wt. %, about 88.0 wt. %, about 89.0 wt. %, about 90.0 wt. %, about 91.0 wt. %, about 92.0 wt. %, about 93.0 wt. %, about 94.0 wt. %, about 95.0 wt. %, about 96.0 wt. %, about 97.0 wt. %, about 98.0 wt. %, by the total weight of the composition.

In some embodiments, the silk containing emulsifier system is present in the aqueous phase.

In some embodiments, the collagen stimulating compositions and methods of making and using thereof comprise viscosity modifiers and/or thickeners. In some embodiments, the thickener is selected from the group consisting of ethylene glycol monostearate, carbomer polymers, carboxyvinyl polymer, acrylic copolymers, methyl cellulose, copolymers of lactide and glycolide monomers, ethyl cellulose, hydroxyethyl cellulose, hydroxypropyl cellulose, carrageenan, hydrophobically modified hydroxy-ethyl-cellulose, laponite and water soluble salts of cellulose ethers such as sodium carboxymethylcellulose and sodium carboxymethyl hydroxyethyl cellulose, natural gums such as gum karaya, gum arabic, Guars, HP Guars, heteropolysaccharide gums (e.g., xanthan gum), and gum tragacanth.

In some embodiments, the thickener is selected from the group consisting of talc, fumed silica, polymeric polyether compound (e.g., polyethylene or polypropylene oxide (MW 300 to 1,000,000), capped with alkyl or acyl groups containing 1 to about 18 carbon atoms), ethylene glycol stearate, alkanolamides of fatty acids having from 16 to 22 carbon atoms, polyethylene glycol 3 distearate, polyacrylic acids (e.g., Carbopol® 420, Carbopol® 488 or Carbopol® 493), cross-linked polymers of acrylic acid, copolymers of acrylic acid with a hydrophobic monomer, copolymers of carboxylic acid-containing monomers and acrylic esters (e.g. Carbopol® 1342), cross-linked copolymers of acrylic acid and acrylate esters, polyacrylic acids cross-linked with polyfunctional agent (e.g., Carbopol® 910, Carbopol® 934, Carbopol® 940, Carbopol® 941 and Carbopol® 980, Ultrez® 10), and crystalline long chain acyl derivatives.

In some embodiments, the collagen stimulating compositions and methods of making and using thereof comprise from about 0.1 wt. % to about 15.0 wt. % of thickener/viscosity modifying agent by the total weight of the composition. In some embodiments, the collagen stimulating compositions and methods of making and using thereof comprise from about 0.1 wt. % to about 10.0 wt. % of thickener/viscosity modifying agent by the total weight of the composition. In some embodiments, the collagen stimulating compositions and methods of making and using thereof comprise from about 0.5 wt. % to about 6.0 wt. % of thickener/viscosity modifying agent by the total weight of the composition. In some embodiments, the collagen stimulating compositions and methods of making and using thereof comprise from about 0.9 wt. % to about 4.0 wt. % of thickener/viscosity modifying agent by the total weight of the composition. In some embodiments, the collagen stimulating compositions and methods of making and using thereof comprise about 2.0 wt. % of thickener/viscosity modifying agent by the total weight of the composition. In some embodiments, the amount of the thickener/viscosity modifying agent presented in the collagen stimulating compositions and methods of making and using thereof is selected from the group consisting of about 0.1 wt. %, about 0.2 wt. %, about 0.3 wt. %, about 0.4 wt. %, about 0.5 wt. %, about 0.6 wt. %, about 0.7 wt. %, about 0.8 wt. 10%, about 0.9 wt. %, about 1.0 wt. %, about 1.25 wt. %, about 1.50 wt. %, about 1.75 wt. %, about 2.0 wt. %, about 2.25 wt. %, about 2.5 wt. %, about 2.75 wt. %, about 3.0 wt. %, about 3.25 wt. %, about 3.5 wt. %, about 3.75 wt. %, about 4.0 wt. %, about 4.25 wt. %, about 4.5 wt. %, about 4.75 wt. %, about 5.0 wt. %, about 5.25 wt. %, about 5.5 wt. %, about 5.75 wt. %, about 6.0 wt. %, about 6.25 wt. %, about 7.5 wt. %, about 7.75 wt. %, about 8.0 wt. %, about 8.25 wt. %, about 8.5 wt. %, about 8.75 wt. %, about 9.0 wt. %, about 9.25 wt. %, about 9.5 wt. %, about 9.75 wt. %, about 10.0 wt. %, about 10.1 wt. %, about 10.2 wt. %, about 10.3 wt. %, about 10.4 wt. %, about 10.5 wt. %, about 10.6 wt. %, about 10.7 wt. %, about 10.8 wt. %, about 10.9 wt. %, about 11.0 wt. %, about 11.1 wt. %, about 11.2 wt. %, about 11.3 wt. %, about 11.4 wt. %, about 11.5 wt. %, about 11.6 wt. %, about 11.7 wt. %, about 11.8 wt. %, about 11.9 wt. %, about 12.0 wt. %, about 12.1 wt. %, about 12.2 wt. %, about 12.3 wt. %, about 12.4 wt. %, about 12.5 wt. %, about 12.6 wt. %, about 12.7 wt. %, about 12.8 wt. %, about 12.9 wt. %, about 13.0 wt. %, about 13.1 wt. %, about 13.2 wt. %, about 13.3 wt. %, about 13.4 wt. %, about 13.5 wt. %, about 13.6 wt. %, about 13.7 wt. %, about 13.8 wt. %, about 13.9 wt. %, about 14.0 wt. %, about 14.1 wt. %, about 14.2 wt. %, about 14.3 wt. %, about 14.4 wt. %, about 14.5 wt. %, about 14.6 wt. %, about 14.7 wt. %, about 14.8 wt. %, about 14.9 wt. %, about 15.0 wt. %, by the total weight of the composition.

In some embodiments, the collagen stimulating compositions and methods of making and using thereof comprise water, an aqueous solution, an alcohol, a blend of alcohol and water, or a lyotropic liquid crystalline phase as aqueous carrier. Selection of the water contained in the composition is not limited in particular; specific examples include purified water, ion-exchanged water, and tap water. In some embodiments, the collagen stimulating compositions and methods of making and using thereof further comprise one or more small molecule polyhydric alcohols selected from the group consisting of ethanediol, propanediol, glycerol, butanediol, butantetraol, xylitol, sorbitol, inositol, ethylene glycol, polyethylene glycol. In some embodiments, the collagen stimulating compositions and methods of making and using thereof further comprise one or more low alcohol solvent including methanol, ethanol, and isopropanol.

In some embodiments, the collagen stimulating compositions and methods of making and using thereof comprise from about 50 wt. % to about 98 wt. % of the aqueous carrier by the total weight of the composition. In some embodiments, the collagen stimulating compositions and methods of making and using thereof comprise from about 60 wt. % to about 90 wt. % of the aqueous carrier by the total weight of the composition. In some embodiments, the amount of the aqueous carrier in the collagen stimulating compositions and methods of making and using thereof is selected from: about 50.0 wt. %, about 51.0 wt. %, about 52.0 wt. %, about 53.0 wt. %, about 54.0 wt. %, about 55.0 wt. %, about 56.0 wt. %, about 57.0 wt. %, about 58.0 wt. %, about 59.0 wt. %, about 60.0 wt. %, about 61.0 wt. %, about 62.0 wt. %, about 63.0 wt. %, about 64.0 wt. %, about 65.0 wt. %, about 66.0 wt. %, about 67.0 wt. %, about 68.0 wt. %, about 69.0 wt. %, about 70.0 wt. %, about 71.0 wt. %, about 72.0 wt. %, about 73.0 wt. %, about 74.0 wt. %, about 75.0 wt. %, about 76.0 wt. %, about 77.0 wt. %, about 78.0 wt. %, about 79.0 wt. %, about 80.0 wt. %, about 81.0 wt. %, about 82.0 wt. %, about 83.0 wt. %, about 84.0 wt. %, about 85.0 wt. %, about 86.0 wt. %, about 87.0 wt. %, about 88.0 wt. %, about 89.0 wt. %, about 90.0 wt. %, about 91.0 wt. %, about 92.0 wt. %, about 93.0 wt. %, about 94.0 wt. %, about 95.0 wt. %, about 96.0 wt. %, about 97.0 wt. %, about 98.0 wt. %, by the total weight of the composition.

In some embodiments, the collagen stimulating compositions and methods of making and using thereof comprise a non-aqueous liquid carrier. Non-aqueous liquid carrier as used herein means that the liquid carrier is substantially free of water. In the present invention, “the liquid carrier being substantially free of water” means that: the liquid carrier is free of water; or, if the liquid carrier contains water, the level of water is very low. In the present invention, the level of water, if included, 1% or less, preferably 0.5% or less, more preferably 0.3% or less, still more preferably 0.1% or less, even more preferably 0% by weight of the composition.

In some embodiments, the non-aqueous liquid carrier comprises an oily material selected from the group consisting of mineral oil, hydrocarbon oils, hydrogenated polydecene, polyisobutene, isoparaffin, isododecane, isohexadecane, volatile silicone oil, non-volatile silicone oil, isohexadecane, squalene, squalene, ester oil and combination thereof. In some embodiments, the non-aqueous liquid carrier comprises an oily material selected from the group consisting of white mineral oils, squalane, hydrogenated polyisobutene, isohexadecane, and isododecane. In some embodiments, the non-aqueous liquid carrier comprises squalane and hydrogenated polyisobutene. In some embodiments, the non-aqueous liquid carrier comprises white mineral oils, isohexadecane, and isododecane. In some embodiments, the hydrocarbon oil is selected from the group consisting of liquid paraffin, liquid isoparaffin, squalene, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, permethyl-substituted isomers, e.g., the permethyl-substituted isomers of hexadecane and eicosane (e.g., 2,2,4,4,6,6,8,8-dimethyl-10-methylundecane and 2,2,4,4,6,6-dimethyl-8-methylnonane), copolymer of isobutylene and butane, poly-α-olefins (e.g., polymer of ethylene, propylene, 1-butene, 1-pentene, 1-hexene, 1-octene, 1-decene, 1-dodecene, 1-tetradecene), and combination thereof.

In some embodiments, the collagen stimulating compositions and methods of making and using thereof comprise an organic oil comprising a fatty ester oil selected from the group consisting of isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, oleyl adipate, isopropyl myristate, glycol stearate, and isopropyl laurate, isocetyl stearoyl stearate, diisopropyl adipate, tristearyl citrate, triolein, tristearin glyceryl dilaurate, C₈-C₁₀ triester of trimethylolpropane, tetraester of 3,3 diethanol-1,5 pentadiol, C₈-C₁₀ diester of adipic acid, ethylene glycol mono and di-fatty acid esters, diethylene glycol mono- and di-fatty acid esters, polyethylene glycol mono- and di-fatty acid esters, propylene glycol mono- and di-fatty acid esters, polypropylene glycol monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, sorbitan fatty acid esters, and polyoxyethylene sorbitan fatty acid esters (e.g. polyoxyethylene (20) sorbitan monooleate, polysorbate 80, Tween 80®), and combination thereof.

In some embodiments, the non-aqueous liquid carrier comprises a volatile isoparaffin having from about 8 to about 20 carbon atoms. In some embodiments, the non-aqueous liquid carrier comprises a volatile isoparaffin having from about 8 to about 16 carbon atoms. In some embodiments, the non-aqueous liquid carrier comprises a volatile isoparaffin having from about 10 to about 16 carbon atoms. In some embodiments, the volatile isoparaffin is selected from the group consisting of trimer, tetramer, and pentamer of isobutene, and mixtures thereof. Commercially available isoparaffin hydrocarbons may have distributions of its polymerization degree, and may be mixtures of, for example, trimer, tetramer, and pentamer. What is meant by tetramer herein is that a commercially available isoparaffin hydrocarbons in which tetramer has the highest content, i.e., tetramer is included at a level of preferably 70% or more, more preferably 80% or more, still more preferably 85% or more.

In some embodiments, the volatile isoparaffin is a mixture of several grades of isoparaffins. In some embodiments, the volatile isoparaffin has a viscosity range selected from: about 0.5 mm²·s⁻¹ to about 50 mm²·s⁻¹, about 0.8 mm²·s⁻¹ to about 40 mm²·s⁻¹, about 1 mm²·s⁻¹ to about 30 mm²·s⁻¹, about 1 mm²·s⁻¹ to about 20 mm²·s⁻¹, and about 1 mm²·s⁻¹ to about 10 mm²·s⁻¹, at 37.8° C. When using two or more isoparaffin hydrocarbon solvents, it is preferred that the mixture of isoparaffin hydrocarbon solvents have the above viscosity.

In some embodiments, the non-aqueous liquid carrier comprises ester oil. In some embodiments, the ester oils have an HLB of 3 or less, and as liquid at room temperature. In some embodiments, the ester oil is selected from the group consisting of methyl palmitate, methyl stearate, methyl oleate, methyl linoleate, and methyl laurate. In an embodiment, the ester oil methyl stearate.

In some embodiments, the ester oil is included in the non-aqueous liquid carrier at a weight percent selected from: about 0.1 wt. % to about 25 wt. %, about 0.5 wt. % to about 15 wt. %, about 1.0 wt. % to about 10 wt. %, about 1.0 wt. % to about 5.0 wt. % by the total weight of the collagen boosting composition, in view of the balance between conditioned feel and product stability, and/or in view of prevent foaming.

In some embodiments, the non-aqueous liquid carrier comprises fatty esters selected from the group consisting of trimethyloylpropane tricaprylate/tricaprylate, C12-C15 alkyl benzoate, ethylhexyl stearate, ethylhexyl cocoate, decyl oleate, decyl cocoate, ethyl oleate, isopropyl myristate, ethylhexyl perlagonate, pentaerythrityl tetracaprylate/tetracaprate, PPG-3 benzyl ether myristate, propyiene glycol dicaprylate/dicaprate, ethylhexyl isostearate, ethylhexyl palmitate and natural oils such as glycine soja, Helianthus annuus, Simmondsia chinensis, Carthamus tinctorius, Oenothera biennis and rapae oleum, and combination thereof.

In some embodiments, the non-aqueous liquid carrier comprises glyceride fatty ester. In some embodiments, the suitable glyceride fatty esters for use in hair oils of the invention have a viscosity at ambient temperature (25 to 30° C.) of from 0.01 to 0.8 Pa·s, preferably from 0.015 to 0.6 Pa·s, more preferably from 0.02 to 0.065 Pa·s.

In an embodiment, the fatty material comprises a glyceride fatty ester. As used herein, the term “glyceride fatty esters” refers to the mono-, di-, and tri-esters formed between glycerol and long chain carboxylic acids such as C6-C30 carboxylic acids.

The carboxylic acids may be saturated or unsaturated or contain hydrophilic groups such as hydroxyl. Preferred glyceride fatty esters are derived from carboxylic acids of carbon chain length ranging from C10 to C24, preferably C10 to C22, most preferably C12 to C 20, most preferably C12 to C18. In some embodiments, glyceride fatty ester is a medium-chain triglyceride having C6-C12 fatty acid chain.

In some embodiments, glyceride fatty ester is sourced from varieties of vegetable and animal fats and oils, such as camellia oil, coconut oil, castor oil, safflower oil, sunflower oil, peanut oil, cottonseed oil, corn oil, olive oil, cod liver oil, almond oil, avocado oil, palm oil, sesame oil, lanolin and soybean oil. Synthetic oils include trimyristin, triolein and tristearin glyceryl dilaurate. Vegetable derived glyceride fatty esters include almond oil, castor oil, coconut oil, palm kernel oil, sesame oil, sunflower oil and soybean oil.

In some embodiments, the glyceride fatty ester is selected from coconut oil, sunflower oil, almond oil and mixtures thereof.

The non-aqueous liquid carrier is included at a level by weight of the collagen boosting composition of, from about 50% to about 99.9%, from about 60% to about 99.8%, more preferably from about 65% to about 98% by the total weight of the collagen boosting composition.

In some embodiments, the collagen stimulating compositions and methods of making and using thereof comprise an aqueous liquid carrier substantially free of non-silk surfactant. In some embodiments, the aqueous liquid carrier is selected from water, an aqueous solution, an alcohol, a blend of alcohol and water, or a lyotropic liquid crystalline phase. Selection of the water contained in the composition is not limited in particular; specific examples include purified water, ion-exchanged water, and tap water.

In some embodiments, the aqueous liquid carrier comprises one or more small molecule polyhydric alcohols selected from the group consisting of ethanediol, propanediol, glycerol, butanediol, butantetraol, xylitol, sorbitol, inositol, ethylene glycol, polyethylene glycol. In some embodiments, the aqueous liquid carrier comprises water and glycerol. In some embodiments, the aqueous liquid carrier comprises water and glycerol in a weight ratio of water to glycerol at 1:10. In some embodiments, the aqueous liquid carrier comprises water and glycerol in a weight ratio of water to glycerol selected from 1:10, 1:9, 1:8, 1:7, 1:6, 1:5, 1:4, 1:3, 1:2, and 1:1. In some embodiments, the aqueous liquid carrier comprises water and glycerol in a weight ratio of water to glycerol at 1:1. In some embodiments, the aqueous liquid carrier comprises water and glycerol in a weight ratio of water to glycerol at 1:10. In some embodiments, the aqueous liquid carrier comprises silk fibroin protein fragments and glycerol in a weight ratio of silk fibroin protein fragments to glycerol selected from 1:10, 1:9, 1:8, 1:7, 1:6, 1:5, 1:4, 1:3, 1:2, and 1:1. In some embodiments, the aqueous liquid carrier comprises silk fibroin protein fragments and glycerol in a weight ratio of silk fibroin protein fragments to glycerol at 1:1.

In some embodiments, the pH of the aqueous liquid phase is adjusted ranging from about 4.0 to about 9.0. In some embodiments, the pH of the aqueous liquid phase is adjusted ranging from about 4.5 to about 8.5. In some embodiments, the pH of the aqueous liquid phase is adjusted ranging from about 5.0 to about 7.0. The pH adjusting agent may include a buffer (e.g. PBS buffer), alkali metal salt, acid, citric acid, succinic acid, phosphoric acid, sodium hydroxide, ammonium hydroxide, ethanolamine, sodium carbonate, and combination thereof.

In some embodiments, the composition comprises from about 1.0 wt. % to about 99.0 wt. % of the aqueous liquid carrier by the total weight of the composition. In some embodiments, the composition comprises from about 5.0 wt. % to about 45.0 wt. % of the aqueous liquid carrier by the total weight of the composition. In some embodiments, the composition comprises from about 5.0 wt. % to about 35.0 wt. % of the aqueous liquid carrier by the total weight of the composition. In some embodiments, the composition comprises from about 10.0 wt. % to about 30.0 wt. % of the aqueous liquid carrier by the total weight of the composition. In some embodiments, the composition comprises from about 45.0 wt. % to about 95.0 wt. % of the aqueous liquid carrier by the total weight of the composition. In some embodiments, the composition comprises from about 60.0 wt. % to about 90.0 wt. % of the aqueous liquid carrier by the total weight of the composition. In some embodiments, the composition comprises from about 45.0 wt. % to about 75.0 wt. % of the aqueous liquid carrier by the total weight of the composition. In some embodiments, the composition comprises from about 60.0 wt. % to about 75.0 wt. % of the aqueous liquid carrier by the total weight of the composition. In some embodiments, the amount of the aqueous liquid carrier in the composition is selected from: about 1.0 wt. %, about 2.0 wt. %, about 3.0 wt. %, about 4.0 wt. %, about 5.0 wt. %, about 6.0 wt. %, about 7.0 wt. %, about 8.0 wt. %, about 9.0 wt. %, about 10.0 wt. %, about 11.0 wt. %, about 12.0 wt. %, about 13.0 wt. %, about 14.0 wt. %, about 15.0 wt. %, about 16.0 wt. %, about 17.0 wt. %, about 18.0 wt. %, about 19.0 wt. %, about 20.0 wt. %, about 21.0 wt. %, about 22.0 wt. %, about 23.0 wt. %, about 24.0 wt. %, about 25.0 wt. %, about 26.0 wt. %, about 27.0 wt. %, about 28.0 wt. %, about 29.0 wt. %, about 30.0 wt. %, about 31.0 wt. %, about 32.0 wt. %, about 33.0 wt. %, about 34.0 wt. %, about 35.0 wt. %, about 36.0 wt. %, about 37.0 wt. %, about 38.0 wt. %, about 39.0 wt. %, about 40.0 wt. %, about 41.0 wt. %, about 42.0 wt. %, about 43.0 wt. %, about 44.0 wt. %, about 45.0 wt. %, about 46.0 wt. %, about 47.0 wt. %, about 48.0 wt. %, about 49.0 wt. %, about 50.0 wt. %, about 51.0 wt. %, about 52.0 wt. %, about 53.0 wt. %, about 54.0 wt. %, about 55.0 wt. %, about 56.0 wt. %, about 57.0 wt. %, about 58.0 wt. %, about 59.0 wt. %, about 60.0 wt. %, about 61.0 wt. %, about 62.0 wt. %, about 63.0 wt. %, about 64.0 wt. %, about 65.0 wt. %, about 66.0 wt. %, about 67.0 wt. %, about 68.0 wt. %, about 69.0 wt. %, about 70.0 wt. %, about 71.0 wt. %, about 72.0 wt. %, about 73.0 wt. %, about 74.0 wt. %, about 75.0 wt. %, about 76.0 wt. %, about 77.0 wt. %, about 78.0 wt. %, about 79.0 wt. %, about 80.0 wt. %, about 81.0 wt. %, about 82.0 wt. %, about 83.0 wt. %, about 84.0 wt. %, about 85.0 wt. %, about 86.0 wt. %, about 87.0 wt. %, about 88.0 wt. %, about 89.0 wt. %, about 90.0 wt. %, about 91.0 wt. %, about 92.0 wt. %, about 93.0 wt. %, about 94.0 wt. %, about 95.0 wt. %, about 96.0 wt. %, about 97.0 wt. %, about 98.0 wt. %, by the total weight of the composition.

In some embodiments, the collagen stimulating compositions and methods of making and using thereof optionally comprise a natural or synthetic fragrant essential oil. In some embodiments, the fragrant essential oil is selected from the group consisting of eucalyptus oil, lavandin oil, lavender oil, vetiver oil, litsea cubeba oil, lemon oil, sandalwood oil, rosemary oil, chamomile oil, savory oil, nutmeg oil, cinnamon oil, hyssop oil, caraway oil, orange oil, geraniol oil, cade oil, almond oil, argan oil, avocado oil, cedar oil, wheat germ oil, bergamot oil, and combination thereof.

In some embodiments, the collagen stimulating compositions and methods of making and using thereof optionally comprise vitamins selected from the group selected from the group consisting of vitamin A, vitamin B, vitamin E, vitamin D, vitamin K, riboflavin, pyridoxin, coenzyme thiamine pyrophosphate, flavin adenine dinucleotide, folic acid, pyridoxal phosphate, tetradrofolic acid, and combination thereof.

In some embodiments, the collagen stimulating compositions and methods of making and using thereof contains vitamin and/or coenzymes at about 0.01 wt. % to about 8.0 wt. % by the total weight of the composition. In some embodiments, the composition contains vitamin and/or coenzymes at about 0.001 wt. % to about 10.0 wt. % by the total weight of the composition. In some embodiments, the composition contains vitamin and/or coenzymes at about 0.05 wt. % to about 5.0 wt. % by the total weight of the composition.

In some embodiments, the collagen stimulating compositions and methods of making and using thereof optionally comprise a preservative selected from the group consisting of triazoles, imidazoles, naphthalene derivatives, benzimidazoles, morphline derivatives, dithiocarbamates, benzisothiazoles, benzamides, boron compounds, formaldehyde donors, isothiazolones, thiocyanates, quaternary ammonium compounds, iodine derivates, phenol derivatives, micobicides, pyridines, dialkylthiocarbamates, nitriles, parabens, alkyl parabens, and salts thereof.

In some embodiments, the collagen stimulating compositions and methods of making and using thereof is formulated in a form selected from the group consisting of aqueous solution, ethanolic solution, oil, gel, emulsion, suspension, mousses, liquid crystal, solid, gels, lotions, creams, aerosol sprays, paste, foam and tonics. In some embodiments, the composition is in a form selected from the group consisting of a cream, spray, aerosol, mousse, or gel.

In an embodiment, the composition may include a solubilizer to ensure good solubilization and/or dissolution of the compound of the present disclosure and to minimize precipitation of the compound of the present disclosure. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, δ-valerolactone and isomers thereof, β-butyrolactone and isomers thereof, and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

The following clauses describe certain embodiments.

Clause 1. An article comprising a fabric and a coating, wherein the coating comprises a surfactant and/or emulsifier system, and silk fibroin fragments having an average weight average molecular weight selected from between about 1 kDa and about 5 kDa, from between about 5 kDa and about 10 kDa, from between about 6 kDa and about 17 kDa, from between about 10 kDa and about 15 kDa, from between about 14 kDa and about 30 kDa, from between about 15 kDa and about 20 kDa, from between about 17 kDa and about 39 kDa, from between about 20 kDa and about 25 kDa, from between about 25 kDa and about 30 kDa, from between about 30 kDa and about 35 kDa, from between about 35 kDa and about 40 kDa, from between about 39 kDa and about 54 kDa, from between about 39 kDa and about 80 kDa, from between about 40 kDa and about 45 kDa, from between about 45 kDa and about 50 kDa, from between about 50 kDa and about 55 kDa, from between about 55 kDa and about 60 kDa, from between about 60 kDa and about 100 kDa, or from between about 80 kDa and about 144 kDa, and a polydispersity ranging from 1 to about 5. In some embodiments, the coating comprises a composition comprising a plurality of peptides or protein fragments, each comprising a plurality of amino acids selected from M, R, V, K, T, F, I, L, C, A, Q, Y, N, D, E, G, S, H, P, and W, wherein at least one of the amino acids is modified, substituted, or replaced. In some embodiments, the plurality of peptides or protein fragments comprises a fibroin peptide or protein fragment comprising an amino acid modification, substitution, or replacement of an amino acid selected from M, R, V, K, T, F, I, L, C, A, Q, Y, N, D, E, G, S, H, P, and W. In some embodiments, the fibroin is a fibroin heavy chain, a fibroin light chain, or a fibrohexamerin. In some embodiments, the peptide or protein fragment comprises between about 2 and about 100 amino acids. In some embodiments, the peptide or protein fragment comprises between about 2 and about 25 amino acids. In some embodiments, the peptide or protein fragment comprises between about 25 and about 50 amino acids. In some embodiments, the peptide or protein fragment comprises between about 50 and about 75 amino acids. In some embodiments, the peptide or protein fragment comprises between about 75 and about 100 amino acids. In some embodiments, the peptide or protein fragment comprises between about 100 and about 125 amino acids. In some embodiments, the peptide or protein fragment comprises between about 125 and about 150 amino acids. In some embodiments, the peptide or protein fragment comprises between about 150 and about 200 amino acids. In some embodiments, the peptide or protein fragment comprises between about 200 and about 250, 300, 350, 400, 450, or 500 amino acids. In some embodiments, the peptide or protein fragment comprises between one and five modifications, substitutions, and/or replacements. In some embodiments, the peptide or protein fragment comprises one modification, substitution, and/or replacement. In some embodiments, the peptide or protein fragment comprises two modifications, substitutions, and/or replacements. In some embodiments, the peptide or protein fragment comprises three modifications, substitutions, and/or replacements. In some embodiments, the peptide or protein fragment comprises four modifications, substitutions, and/or replacements. In some embodiments, the peptide or protein fragment comprises five modifications, substitutions, and/or replacements. In some embodiments, the peptide or protein fragment comprises six modifications, substitutions, and/or replacements. In some embodiments, the peptide or protein fragment comprises seven modifications, substitutions, and/or replacements. In some embodiments, the peptide or protein fragment comprises eight modifications, substitutions, and/or replacements. In some embodiments, the peptide or protein fragment comprises nine modifications, substitutions, and/or replacements. In some embodiments, the peptide or protein fragment comprises ten modifications, substitutions, and/or replacements. In some embodiments, a modification, substitution, and/or replacement is selected from an asparagine to aspartic acid modification, substitution, and/or replacement, a glutamine to glutamic acid modification, substitution, and/or replacement, and a methionine to methionine oxide modification, substitution, and/or replacement. In some embodiments, the fibroin is a fibroin heavy chain, and wherein a modification, substitution, and/or replacement is at a position corresponding to any one position from 1 to 5263 of the fibroin heavy chain. In some embodiments, a modification, substitution, and/or replacement is at Q58, M64, N68, N70, N77, M80, N93, M103, Q125, N132, Q139, Q275, N4191, Q5216, and/or N5262 position of fibroin heavy chain. In some embodiments, the fibroin is a fibroin light chain, and wherein a modification, substitution, and/or replacement is at a position corresponding to any one position from 1 to 262 of the fibroin light chain. In some embodiments, a modification, substitution, and/or replacement is at N23, Q24, N28, M69, N105, N108, N118, N136, N138, Q149, N186, N200, Q202, N204, N240, N248, and/or Q255 position of fibroin light chain. In some embodiments, the fibroin is a fibrohexamerin (p25), and wherein a modification, substitution, and/or replacement is at a position corresponding to any one position from 1 to 220 of the fibrohexamerin (p25). In some embodiments, a modification, substitution, and/or replacement is at Q62, N93, M120, N149, N172, N174, and/or N202 position of fibrohexamerin (p25). In some embodiments, each modification, substitution, and/or replacement is independently ranging in the composition between about 1% to about 99%. In some embodiments, each modification, substitution, and/or replacement is independently ranging in the composition between about 1% to about 10%. In some embodiments, each modification, substitution, and/or replacement is independently ranging in the composition between about 10% to about 20%. In some embodiments, each modification, substitution, and/or replacement is independently ranging in the composition between about 20% to about 30%. In some embodiments, each modification, substitution, and/or replacement is independently ranging in the composition between about 30% to about 40%. In some embodiments, each modification, substitution, and/or replacement is independently ranging in the composition between about 40% to about 50%. In some embodiments, each modification, substitution, and/or replacement is independently ranging in the composition between about 50% to about 60%. In some embodiments, each modification, substitution, and/or replacement is independently ranging in the composition between about 60% to about 70%. In some embodiments, each modification, substitution, and/or replacement is independently ranging in the composition between about 70% to about 80%. In some embodiments, each modification, substitution, and/or replacement is independently ranging in the composition between about 80% to about 90%. In some embodiments, each modification, substitution, and/or replacement is independently ranging in the composition between or about 90% to about 99%. As used herein, a % modification, substitution, and/or replacement is defined as (number of peptide or protein fragments comprising a modification, substitution, and/or replacement at a specific position, divided by the total number of peptide or protein fragments which include the specific position, whether comprising a modification, substitution, and/or replacement, or not)×100. In some embodiments, the coating comprises a composition comprising a plurality of peptides or protein fragments of fibroin heavy chain, fibroin light chain, and/or fibrohexamerin (p25), the composition comprising one or more fractions, wherein the plurality of peptides or protein fragments comprises a fibroin peptide or protein fragment comprising an amino acid modification, substitution, or replacement. In some embodiments, the plurality of peptides or protein fragments having a weight average molecular weight (M_{w}) selected from between about 1 kDa and about 20 kDa, from between about 20 kDa and about 40 kDa, from between about 40 kDa and about 60 kDa, from between about 60 kDa and about 80 kDa, from between about 80 kDa and about 100 kDa, from between about 100 kDa and about 120 kDa, from between about 120 kDa and about 140 kDa, or from between about 140 kDa and about 160 kDa, from between about 160 kDa and about 180 kDa, from between about 180 kDa and about 200 kDa, or from between about 200 kDa and about 250 kDa, and a polydispersity between 1 and about 1.7. In some embodiments, the plurality of peptides or protein fragments in a fraction having a weight average molecular weight (M_{w}) selected from between about 10 kDa and about 20 kDa, from between about 20 kDa and about 40 kDa, from between about 40 kDa and about 60 kDa, from between about 60 kDa and about 80 kDa, from between about 80 kDa and about 100 kDa, from between about 100 kDa and about 120 kDa, from between about 120 kDa and about 140 kDa, from between about 140 kDa and about 160 kDa, or from between about 160 kDa and about 180 kDa, and a polydispersity between 1 and about 1.1, or 1 and about 1.2. In some embodiments, the plurality of peptides or protein fragments in a fraction having a weight average molecular weight (M_{w}) selected from between about 10 kDa and about 20 kDa, from between about 20 kDa and about 40 kDa, from between about 40 kDa and about 60 kDa, from between about 60 kDa and about 80 kDa, from between about 80 kDa and about 100 kDa, from between about 100 kDa and about 120 kDa, or from between about 120 kDa and about 140 kDa, and a polydispersity between 1 and about 1.1, or 1 and about 1.2. In some embodiments, the plurality of peptides or protein fragments in a fraction having a weight average molecular weight (M_{w}) selected from between about 60 kDa and about 80 kDa, from between about 80 kDa and about 100 kDa, or from between about 100 kDa and about 120 kDa, and a polydispersity between 1 and about 1.1. In some embodiments, the plurality of peptides or protein fragments in a fraction having a weight average molecular weight (M_{w}) selected from between about 10 kDa and about 20 kDa, from between about 20 kDa and about 40 kDa, from between about 40 kDa and about 60 kDa, from between about 60 kDa and about 80 kDa, from between about 80 kDa and about 100 kDa, or from between about 100 kDa and about 110 kDa, and a polydispersity between 1 and about 1.1, or 1 and about 1.2. In some embodiments, the plurality of peptides or protein fragments in a fraction having a weight average molecular weight (M_{w}) selected from between about 60 kDa and about 80 kDa, from between about 80 kDa and about 100 kDa, from between about 100 kDa and about 120 kDa, or from between about 120 kDa and about 140 kDa, and a polydispersity between 1 and about 1.1. In some embodiments, the plurality of peptides or protein fragments in a fraction having a weight average molecular weight (M_{w}) selected from between about 20 kDa and about 40 kDa, or from between about 40 kDa and about 60 kDa, and a polydispersity between 1 and about 1.1, or 1 and about 1.2. In some embodiments, the one or more fractions are selected from AS77, AS78, AS79, AS80, and AS81. In some embodiments, the one or more fractions are selected from AS82, AS83, AS84, AS85, AS86, AS87, AS88, and AS89. In some embodiments, the one or more fractions are selected from AS90, AS91, AS92, AS93, and AS94. In some embodiments, the one or more fractions are selected from AS95, AS96, AS97, AS98, AS99, and AS100. In some embodiments, the plurality of peptides or protein fragments having a weight average molecular weight (M_{w}) selected from between about 40 kDa and about 60 kDa, from between about 60 kDa and about 80 kDa, from between about 80 kDa and about 100 kDa, from between about 100 kDa and about 120 kDa, from between about 120 kDa and about 140 kDa, from between about 140 kDa and about 160 kDa, from between about 160 kDa and about 180 kDa, from between about 180 kDa and about 200 kDa, or from between about 200 kDa and about 220 kDa, and a polydispersity between 1 and about 1.7. In some embodiments, the plurality of peptides or protein fragments in a fraction having a weight average molecular weight (M_{w}) selected from between about 40 kDa and about 60 kDa, from between about 60 kDa and about 80 kDa, from between about 80 kDa and about 100 kDa, from between about 100 kDa and about 120 kDa, from between about 120 kDa and about 140 kDa, from between about 140 kDa and about 160 kDa, from between about 160 kDa and about 180 kDa, from between about 180 kDa and about 200 kDa, or from between about 200 kDa and about 210 kDa, and a polydispersity between 1 and about 1.2, or 1 and about 1.3. In some embodiments, the plurality of peptides or protein fragments in a fraction having a weight average molecular weight (M_{w}) selected from between about 40 kDa and about 60 kDa, from between about 60 kDa and about 80 kDa, from between about 80 kDa and about 100 kDa, or from between about 100 kDa and about 110 kDa, and a polydispersity between 1 and about 1.1, or 1 and about 1.2. In some embodiments, the plurality of peptides or protein fragments in a fraction having a weight average molecular weight (M_{w}) selected from between about 60 kDa and about 80 kDa, from between about 80 kDa and about 100 kDa, from between about 100 kDa and about 120 kDa, from between about 120 kDa and about 140 kDa, from between about 140 kDa and about 160 kDa, from between about 160 kDa and about 180 kDa, from between about 180 kDa and about 200 kDa, or from between about 200 kDa and about 210 kDa, and a polydispersity between 1 and about 1.2, or 1 and about 1.3. In some embodiments, the one or more fractions are selected from AS101, AS102, AS103, AS104, and AS105. In some embodiments, the one or more fractions are selected from AS106, AS107, AS108, AS109, AS110, and AS111. In some embodiments, an amino acid is selected from M, R, V, K, T, F, I, L, C, A, Q, Y, N, D, E, G, S, H, P, and W. In some embodiments, a peptide or protein fragment comprises between about 2 and about 100 amino acids. In some embodiments, the peptide or protein fragment comprises between about 2 and about 25 amino acids. In some embodiments, the peptide or protein fragment comprises between about 25 and about 50 amino acids. In some embodiments, the peptide or protein fragment comprises between about 50 and about 75 amino acids. In some embodiments, the peptide or protein fragment comprises between about 75 and about 100 amino acids. In some embodiments, the peptide or protein fragment comprises between about 100 and about 125 amino acids. In some embodiments, the peptide or protein fragment comprises between about 125 and about 150 amino acids. In some embodiments, the peptide or protein fragment comprises between about 150 and about 200 amino acids. In some embodiments, the peptide or protein fragment comprises between about 200 and about 250, 300, 350, 400, 450, or 500 amino acids. In some embodiments, a peptide or protein fragment comprises between one and five modifications, substitutions, and/or replacements. In some embodiments, the peptide or protein fragment comprises one modification, substitution, and/or replacement. In some embodiments, the peptide or protein fragment comprises two modifications, substitutions, and/or replacements. In some embodiments, the peptide or protein fragment comprises three modifications, substitutions, and/or replacements. In some embodiments, the peptide or protein fragment comprises four modifications, substitutions, and/or replacements. In some embodiments, the peptide or protein fragment comprises five modifications, substitutions, and/or replacements. In some embodiments, the peptide or protein fragment comprises six modifications, substitutions, and/or replacements. In some embodiments, the peptide or protein fragment comprises seven modifications, substitutions, and/or replacements. In some embodiments, the peptide or protein fragment comprises eight modifications, substitutions, and/or replacements. In some embodiments, the peptide or protein fragment comprises nine modifications, substitutions, and/or replacements. In some embodiments, the peptide or protein fragment comprises ten modifications, substitutions, and/or replacements. In some embodiments, the fibroin is a fibroin heavy chain, and wherein a modification, substitution, and/or replacement is at a position corresponding to any one position from 1 to 5263 of the fibroin heavy chain. In some embodiments, the fibroin is a fibroin light chain, and wherein a modification, substitution, and/or replacement is at a position corresponding to any one position from 1 to 262 of the fibroin light chain. In some embodiments, the fibroin is a fibrohexamerin (p25) chain, and wherein a modification, substitution, and/or replacement is at a position corresponding to any one position from 1 to 220 of the fibrohexamerin (p25) chain. In some embodiments, a modification, substitution, and/or replacement is selected from an asparagine to aspartic acid modification, substitution, and/or replacement, a glutamine to glutamic acid modification, substitution, and/or replacement, and a methionine to methionine oxide modification, substitution, and/or replacement. In some embodiments, a modification, substitution, and/or replacement is at fibroin heavy chain position selected from Q58, M64, N68, N70, N77, M80, N93, M103, Q125, N132, Q139, Q275, N4191, Q5216, and/or N5262. In some embodiments, a modification, substitution, and/or replacement is at fibroin light chain position selected from N23, Q24, N28, M69, N105, N108, N118, N136, N138, Q149, N186, N200, Q202, N204, N240, N248, and/or Q255. In some embodiments, a modification, substitution, and/or replacement is at fibrohexamerin (p25) position selected from Q62, N93, M120, N149, N172, N174, and/or N202. In some embodiments, each modification, substitution, and/or replacement is independently ranging in the composition between about 1% to about 99%. In some embodiments, each modification, substitution, and/or replacement is independently ranging in the composition between about 1% to about 10%. In some embodiments, each modification, substitution, and/or replacement is independently ranging in the composition between about 10% to about 20%. In some embodiments, each modification, substitution, and/or replacement is independently ranging in the composition between about 20% to about 30%. In some embodiments, each modification, substitution, and/or replacement is independently ranging in the composition between about 30% to about 40%. In some embodiments, each modification, substitution, and/or replacement is independently ranging in the composition between about 40% to about 50%. In some embodiments, each modification, substitution, and/or replacement is independently ranging in the composition between about 50% to about 60%. In some embodiments, each modification, substitution, and/or replacement is independently ranging in the composition between about 60% to about 70%. In some embodiments, each modification, substitution, and/or replacement is independently ranging in the composition between about 70% to about 80%. In some embodiments, each modification, substitution, and/or replacement is independently ranging in the composition between about 80% to about 90%. In some embodiments, each modification, substitution, and/or replacement is independently ranging in the composition between or about 90% to about 99%. As used herein, a % modification, substitution, and/or replacement is defined as (number of peptide or protein fragments comprising a modification, substitution, and/or replacement at a specific position, divided by the total number of peptide or protein fragments which include the specific position, whether comprising a modification, substitution, and/or replacement, or not)×100. In some embodiments, a molecular weight is determined by MALS.

Clause 2. The article of clause 1, wherein the silk fibroin fragments have a polydispersity from 1 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, or from about 4.5 to about 5.0.

Clause 3. The article of clause 1, wherein the silk fibroin fragments have a polydispersity from about 1.5 to about 3.0.

Clause 4. The article of clause 1, wherein the silk fibroin fragments comprise one or more of low molecular weight silk fibroin fragments and medium molecular weight silk fibroin fragments.

Clause 5. The article of any one of clauses 1 to 4, further comprising about 0.01% (w/w) to about 10% (w/w) sericin relative to the silk fibroin fragments.

Clause 6. The article of any one of clauses 1 to 5, wherein the fabric comprises one or more of polyester, polyamide, polyaramid, polytetrafluoroethylene, polyethylene, polypropylene, polyurethane, silicone, mixtures of polyurethane and polyethyleneglycol, ultrahigh molecular weight polyethylene, high-performance polyethylene, nylon, LYCRA (polyester-polyurethane copolymer, also known as SPANDEX and elastomer), or a mixture thereof.

Clause 7. The article of any one of clauses 1 to 6, wherein the coating further comprises one or more of a wetting agent, an anti-foaming agent, a softener, a wicking agent, and an anti-microbial.

Clause 8. The article of any one of clauses 1 to 7, wherein the w/w ratio of silk fibroin fragments to the surfactant and/or emulsifier system in the coating is about 99:1, about 98:2, about 97:3, about 96:4, about 95:5, about 94:6, about 93:7, about 92:8, about 91:9, about 90:10, about 89:11, about 88:12, about 87:13, about 86:14, about 85:15, about 84:16, about 83:17, about 82:18, about 81:19, about 80:20, about 79:21, about 78:22, about 77:23, about 76:24, about 75:25, about 74:26, about 73:27, about 72:28, about 71:29, about 70:30, about 69:31, about 68:32, about 67:33, about 66:34, about 65:35, about 64:36, about 63:37, about 62:38, about 61:39, about 60:40, about 59:41, about 58:42, about 57:43, about 56:44, about 55:45, about 54:46, about 53:47, about 52:48, about 51:49, about 50:50, about 49:51, about 48:52, about 47:53, about 46:54, about 45:55, about 44:56, about 43:57, about 42:58, about 41:59, about 40:60, about 39:61, about 38:62, about 37:63, about 36:64, about 35:65, about 34:66, about 33:67, about 32:68, about 31:69, about 30:70, about 29:71, about 28:72, about 27:73, about 26:74, about 25:75, about 24:76, about 23:77, about 22:78, about 21:79, about 20:80, about 19:81, about 18:82, about 17:83, about 16:84, about 15:85, about 14:86, about 13:87, about 12:88, about 11:89, about 10:90, about 9:91, about 8:92, about 7:93, about 6:94, about 5:95, about 4:96, about 3:97, about 2:98, or about 1:99.

Clause 9. The article of any one of clauses 1 to 7, wherein the w/w ratio of silk fibroin fragments to the surfactant and/or emulsifier in the coating is about 1:1, about 1:2, about 1:4, about 1:8, about 1:16, or about 1:32.

Clause 10. The article of any one of clauses 1 to 7, wherein the w/w ratio of silk fibroin fragments to the surfactant and/or emulsifier system in the coating is about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24, about 1:25, about 1:26, about 1:27, about 1:28, about 1:29, about 1:30, about 1:31, or about 1:32.

Clause 11. The article of any one of clauses 1 to 10, wherein the surfactant and/or emulsifier system comprises one or more of polyoxyethylene sorbitan monooleate, polyoxyethylene sorbitan trioleate, polyoxyethylene castor oil, and any combination thereof.

Clause 12. The article of any one of clauses 1 to 10, wherein the surfactant and/or emulsifier system comprises one or more of polyoxyethylene (10-30) sorbitan monooleate, polyoxyethylene (10-30) sorbitan trioleate, polyoxyethylene (10-50) castor oil, and any combination thereof.

Clause 13. The article of any one of clauses 1 to 10, wherein the surfactant and/or emulsifier system comprises one or more of polyoxyethylene (20) sorbitan monooleate, polyoxyethylene (20) sorbitan trioleate, polyoxyethylene (29) castor oil, and any combination thereof.

Clause 14. The article of any one of clauses 1 to 10, wherein the surfactant and/or emulsifier system comprises one or more of polyoxyethylene (20) sorbitan monolaurate, polyoxyethylene (20) sorbitan monopalmitate, polyoxyethylene (20) sorbitan monostearate, polyoxyethylene (20) sorbitan tristearate, and any combination thereof.

Clause 15. The article of any one of clauses 1 to 10, wherein the surfactant and/or emulsifier system comprises one or more of a sorbitan mono fatty acid, a sorbitan tri fatty acid, a castor oil, and any combination thereof.

Clause 16. The article of any one of clauses 1 to 15, wherein the surfactant and/or emulsifier system comprises one or more of coco glucoside, decyl glucoside, lauryl glucoside, sucrose cocoate, capryl/caprylyl glucoside, caprylyl/capryl glucoside, and any combination thereof.

Clause 17. The article of any one of clauses 1 to 16, wherein the surfactant and/or emulsifier system has an HLB between about 11 and about 13.50.

Clause 18. The article of any one of clauses 1 to 16, wherein the surfactant and/or emulsifier system has an HLB between about 11 and about 11.50, between about 11.50 and about 12, between about 12 and about 12.50, between about 12.50 and about 13, or between about 13 and about 13.50. In some embodiments, the surfactant and/or emulsifier system has an HLB of about 11, about 11.1, about 11.2, about 11.3, about 11.4, about 11.5, about 11.6, about 11.7, about 11.8, about 11.9, about 12, about 12.1, about 12.2, about 12.3, about 12.4, about 12.5, about 12.6, about 12.7, about 12.8, about 12.9, about 13, about 13.1, about 13.2, about 13.3, about 13.4, about 13.5, about 13.6, about 13.7, about 13.8, about 13.9, or about 14.

Clause 19. The article of any one of clauses 1 to 18, wherein the article has an improved moisture management comparative to a similar article comprising a similar fabric but no coating.

Clause 20. The article of clause 19, wherein moisture management is assessed by a water absorbency test, a vertical wicking test, or a dry rate test.

Clause 21. The article of any one of clauses 1 to 20, wherein the article has an improved drapability comparative to a similar article comprising a similar fabric but no coating.

Clause 22. The article of any one of clauses 1 to 21, wherein the article has an improved smoothness comparative to a similar article comprising a similar fabric but no coating.

Clause 23. The article of any one of clauses 1 to 22, wherein the article has an improved hand feel comparative to a similar article comprising a similar fabric but no coating.

Clause 24. The article of any one of clauses 1 to 23, wherein the article has a lower charge density at a given pH value comparative to a similar article comprising a similar fabric but no coating.

Clause 25. A method of making a silk fibroin coated fabric, comprising: applying to the fabric a solution comprising a surfactant and/or emulsifier system; applying to the fabric a silk fibroin fragments solution; and drying the fabric.

Clause 26. A method of making a silk fibroin coated fabric, comprising: applying to the fabric a solution comprising a surfactant and/or emulsifier system and silk fibroin fragments disclosed herein; and drying the fabric.

Clause 27. The method of clause 25 or clause 26, wherein the concentration of the silk fibroin fragments in a solution ranges from 0.01 g/L to about 100 g/L.

Clause 28. The method of any one of clauses 25 to 27, wherein the concentration of the surfactant and/or emulsifier system in a solution ranges from 0.01 g/L to about 100 g/L.

Clause 29. The method of any one of clauses 25 to 28, wherein the silk fibroin fragments have an average weight average molecular weight selected from between about 1 kDa and about 5 kDa, from between about 5 kDa and about 10 kDa, from between about 6 kDa and about 17 kDa, from between about 10 kDa and about 15 kDa, from between about 14 kDa and about 30 kDa, from between about 15 kDa and about 20 kDa, from between about 17 kDa and about 39 kDa, from between about 20 kDa and about 25 kDa, from between about 25 kDa and about 30 kDa, from between about 30 kDa and about 35 kDa, from between about 35 kDa and about 40 kDa, from between about 39 kDa and about 54 kDa, from between about 39 kDa and about 80 kDa, from between about 40 kDa and about 45 kDa, from between about 45 kDa and about 50 kDa, from between about 50 kDa and about 55 kDa, from between about 55 kDa and about 60 kDa, from between about 60 kDa and about 100 kDa, or from between about 80 kDa and about 144 kDa, and a polydispersity ranging from 1 to about 5.

Clause 30. The method of any one of clauses 25 to 29, wherein the silk fibroin fragments have a polydispersity from 1 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, or from about 4.5 to about 5.0.

Clause 31. The method of any one of clauses 25 to 29, wherein the silk fibroin fragments have a polydispersity from about 1.5 to about 3.0.

Clause 32. The method of any one of clauses 25 to 29, wherein the silk fibroin fragments comprise one or more of low molecular weight silk fibroin fragments and medium molecular weight silk fibroin fragments.

Clause 33. The method of any one of clauses 25 to 32, wherein a solution further comprises about 0.01% (w/w) to about 10% (w/w) sericin relative to the silk fibroin fragments.

Clause 34. The method of any one of clauses 25 to 33, wherein the fabric comprises one or more of polyester, polyamide, polyaramid, polytetrafluoroethylene, polyethylene, polypropylene, polyurethane, silicone, mixtures of polyurethane and polyethyleneglycol, ultrahigh molecular weight polyethylene, high-performance polyethylene, nylon, LYCRA (polyester-polyurethane copolymer, also known as SPANDEX and elastomer), or a mixture thereof.

Clause 35. The method of any one of clauses 25 to 34, wherein a solution further comprises one or more of a wetting agent, an anti-foaming agent, a softener, a wicking agent, and an anti-microbial.

Clause 36. The method of any one of clauses 25 to 35, wherein the w/w ratio of silk fibroin fragments to the surfactant and/or emulsifier system is about 99:1, about 98:2, about 97:3, about 96:4, about 95:5, about 94:6, about 93:7, about 92:8, about 91:9, about 90:10, about 89:11, about 88:12, about 87:13, about 86:14, about 85:15, about 84:16, about 83:17, about 82:18, about 81:19, about 80:20, about 79:21, about 78:22, about 77:23, about 76:24, about 75:25, about 74:26, about 73:27, about 72:28, about 71:29, about 70:30, about 69:31, about 68:32, about 67:33, about 66:34, about 65:35, about 64:36, about 63:37, about 62:38, about 61:39, about 60:40, about 59:41, about 58:42, about 57:43, about 56:44, about 55:45, about 54:46, about 53:47, about 52:48, about 51:49, about 50:50, about 49:51, about 48:52, about 47:53, about 46:54, about 45:55, about 44:56, about 43:57, about 42:58, about 41:59, about 40:60, about 39:61, about 38:62, about 37:63, about 36:64, about 35:65, about 34:66, about 33:67, about 32:68, about 31:69, about 30:70, about 29:71, about 28:72, about 27:73, about 26:74, about 25:75, about 24:76, about 23:77, about 22:78, about 21:79, about 20:80, about 19:81, about 18:82, about 17:83, about 16:84, about 15:85, about 14:86, about 13:87, about 12:88, about 11:89, about 10:90, about 9:91, about 8:92, about 7:93, about 6:94, about 5:95, about 4:96, about 3:97, about 2:98, or about 1:99.

Clause 37. The method of any one of clauses 25 to 35, wherein the w/w ratio of silk fibroin fragments to the surfactant and/or emulsifier system is about 1:1, about 1:2, about 1:4, about 1:8, about 1:16, or about 1:32.

Clause 38. The method of any one of clauses 25 to 35, wherein the w/w ratio of silk fibroin fragments to the surfactant and/or emulsifier system is about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24, about 1:25, about 1:26, about 1:27, about 1:28, about 1:29, about 1:30, about 1:31, or about 1:32.

Clause 39. The method of any one of clauses 25 to 38, wherein the surfactant and/or emulsifier system comprises one or more of polyoxyethylene sorbitan monooleate, polyoxyethylene sorbitan trioleate, polyoxyethylene castor oil, and any combination thereof.

Clause 40. The method of any one of clauses 25 to 38, wherein the surfactant and/or emulsifier system comprises one or more of polyoxyethylene (10-30) sorbitan monooleate, polyoxyethylene (10-30) sorbitan trioleate, polyoxyethylene (10-50) castor oil, and any combination thereof.

Clause 41. The method of any one of clauses 25 to 38, wherein the surfactant and/or emulsifier system comprises one or more of polyoxyethylene (20) sorbitan monooleate, polyoxyethylene (20) sorbitan trioleate, polyoxyethylene (29) castor oil, and any combination thereof.

Clause 42. The method of any one of clauses 25 to 38, wherein the surfactant and/or emulsifier system comprises one or more of polyoxyethylene (20) sorbitan monolaurate, polyoxyethylene (20) sorbitan monopalmitate, polyoxyethylene (20) sorbitan monostearate, polyoxyethylene (20) sorbitan tristearate, and any combination thereof.

Clause 43. The method of any one of clauses 25 to 38, wherein the surfactant and/or emulsifier system comprises one or more of a sorbitan mono fatty acid, a sorbitan tri fatty acid, a castor oil, and any combination thereof.

Clause 44. The method of any one of clauses 25 to 43, wherein the surfactant and/or emulsifier system comprises one or more of coco glucoside, decyl glucoside, lauryl glucoside, sucrose cocoate, capryl/caprylyl glucoside, caprylyl/capryl glucoside, and any combination thereof.

Clause 45. The method of any one of clauses 25 to 44, wherein the surfactant and/or emulsifier system has an HLB between about 11 and about 13.50.

Clause 46. The method of any one of clauses 25 to 44, wherein the surfactant and/or emulsifier system has an HLB between about 11 and about 11.50, between about 11.50 and about 12, between about 12 and about 12.50, between about 12.50 and about 13, or between about 13 and about 13.50. In some embodiments, the surfactant and/or emulsifier system has an HLB of about 11, about 11.1, about 11.2, about 11.3, about 11.4, about 11.5, about 11.6, about 11.7, about 11.8, about 11.9, about 12, about 12.1, about 12.2, about 12.3, about 12.4, about 12.5, about 12.6, about 12.7, about 12.8, about 12.9, about 13, about 13.1, about 13.2, about 13.3, about 13.4, about 13.5, about 13.6, about 13.7, about 13.8, about 13.9, or about 14.

Clause 47. The method of any one of clauses 25 to 46, wherein the drying comprises heating.

Clause 48. The method of any one of clauses 25 to 47, wherein the pH of a solution is acidic.

Clause 49. The method of any one of clauses 25 to 47, wherein the pH of a solution is between about 3.5 and about 4, between about 4 and about 4.5, between about 4.5 and about 5, between about 5 and about 5.5, or between about 5.5 and about 6. In some embodiments, the pH of a solution is about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, or about 6.

Clause 50. An article prepared by the method of any one of clauses 25 to 49.

Clause 51. The article of clause 50, wherein the article has an improved moisture management comparative to a similar article comprising a similar fabric but no coating.

Clause 52. The article of clause 51, wherein moisture management is assessed by a water absorbency test, a vertical wicking test, or a dry rate test.

Clause 53. The article of any one of clauses 50 to 52, wherein the article has an improved drapability comparative to a similar article comprising a similar fabric but no coating.

Clause 54. The article of any one of clauses 50 to 53, wherein the article has an improved smoothness comparative to a similar article comprising a similar fabric but no coating.

Clause 55. The article of any one of clauses 50 to 54, wherein the article has an improved hand feel comparative to a similar article comprising a similar fabric but no coating.

Clause 56. The article of any one of clauses 50 to 55, wherein the article has a lower charge density at a given pH value comparative to a similar article comprising a similar fabric but no coating.

Clause 57. The article of any one of clauses 1 to 24, or 50 to 56, wherein the amount of silk fibroin fragments in the article is between about 0.01 g to 0.5 g per 1500 m² (denier) to 4000 m² (denier).

Clause 58. The article of any one of clauses 1 to 24, or 50 to 56, wherein the amount of silk fibroin fragments in the article is between about 0.03 g to 0.35 g per 1500 m² (denier) to 4000 m² (denier).

Clause 59. The article of any one of clauses 1 to 24, or 50 to 56, wherein the amount of silk fibroin fragments in the article is between about 0.05 g to 0.2 g per 3000 m² (denier) to 4000 m² (denier).

Clause 60. The article of any one of clauses 1 to 24, or 50 to 56, wherein the amount of silk fibroin fragments in the article is between about 0.2 g to 0.35 g per 1000 m² (denier) to 2500 m² (denier).

Clause 61. The article of any one of clauses 1 to 24, or 50 to 56, wherein the amount of silk fibroin fragments in the article is about 0.01 g/1000-4500 m² (denier), about 0.02 g/1000-4500 m² (denier), about 0.03 g/1000-4500 m² (denier), about 0.04 g/1000-4500 m² (denier), about 0.05 g/1000-4500 m² (denier), about 0.06 g/1000-4500 m² (denier), about 0.07 g/1000-4500 m² (denier), about 0.08 g/1000-4500 m² (denier), about 0.09 g/1000-4500 m² (denier), about 0.10 g/1000-4500 m² (denier), about 0.11 g/1000-4500 m² (denier), about 0.12 g/1000-4500 m² (denier), about 0.13 g/1000-4500 m² (denier), about 0.14 g/1000-4500 m² (denier), about 0.15 g/1000-4500 m² (denier), about 0.16 g/1000-4500 m² (denier), about 0.17 g/1000-4500 m² (denier), about 0.18 g/1000-4500 m² (denier), about 0.19 g/1000-4500 m² (denier), about 0.2 g/1000-4500 m² (denier), about 0.21 g/1000-4500 m² (denier), about 0.22 g/1000-4500 m² (denier), about 0.23 g/1000-4500 m² (denier), about 0.24 g/1000-4500 m² (denier), about 0.25 g/1000-4500 m² (denier), about 0.26 g/1000-4500 m² (denier), about 0.27 g/1000-4500 m² (denier), about 0.28 g/1000-4500 m² (denier), about 0.29 g/1000-4500 m² (denier), about 0.3 g/1000-4500 m² (denier), about 0.31 g/1000-4500 m² (denier), about 0.32 g/1000-4500 m² (denier), about 0.33 g/1000-4500 m² (denier), about 0.34 g/1000-4500 m² (denier), about 0.35 g/1000-4500 m² (denier), about 0.36 g/1000-4500 m² (denier), about 0.37 g/1000-4500 m² (denier), about 0.38 g/1000-4500 m² (denier), about 0.39 g/1000-4500 m² (denier), about 0.4 g/1000-4500 m² (denier), about 0.41 g/1000-4500 m² (denier), about 0.42 g/1000-4500 m² (denier), about 0.43 g/1000-4500 m² (denier), about 0.44 g/1000-4500 m² (denier), about 0.45 g/1000-4500 m² (denier), about 0.46 g/1000-4500 m² (denier), about 0.47 g/1000-4500 m² (denier), about 0.48 g/1000-4500 m² (denier), about 0.49 g/1000-4500 m² (denier), or about 0.5 g/1000-4500 m² (denier).

Clause 101. An article comprising a fabric and a coating, wherein the coating comprises a surfactant and silk fibroin fragments having an average weight average molecular weight selected from between about 1 kDa and about 5 kDa, from between about 5 kDa and about 10 kDa, from between about 6 kDa and about 17 kDa, from between about 10 kDa and about 15 kDa, from between about 14 kDa and about 30 kDa, from between about 15 kDa and about 20 kDa, from between about 17 kDa and about 39 kDa, from between about 20 kDa and about 25 kDa, from between about 25 kDa and about 30 kDa, from between about 30 kDa and about 35 kDa, from between about 35 kDa and about 40 kDa, from between about 39 kDa and about 54 kDa, from between about 39 kDa and about 80 kDa, from between about 40 kDa and about 45 kDa, from between about 45 kDa and about 50 kDa, from between about 50 kDa and about 55 kDa, from between about 55 kDa and about 60 kDa, from between about 60 kDa and about 100 kDa, or from between about 80 kDa and about 144 kDa, and a polydispersity ranging from 1 to about 5.

Clause 102. The article of clause 101, wherein the silk fibroin fragments have a polydispersity from 1 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, or from about 4.5 to about 5.0.

Clause 103. The article of clause 101, wherein the silk fibroin fragments have a polydispersity from about 1.5 to about 3.0.

Clause 104. The article of clause 101, wherein the silk fibroin fragments comprise one or more of low molecular weight silk fibroin fragments and medium molecular weight silk fibroin fragments.

Clause 105. The article of any one of clauses 101-104, further comprising about 0.01% (w/w) to about 10% (w/w) sericin relative to the silk fibroin fragments.

Clause 66. The article of any one of clauses 101-105, wherein the fabric comprises one or more of polyester, polyamide, polyaramid, polytetrafluoroethylene, polyethylene, polypropylene, polyurethane, silicone, mixtures of polyurethane and polyethyleneglycol, ultrahigh molecular weight polyethylene, high-performance polyethylene, nylon, LYCRA (polyester-polyurethane copolymer, also known as SPANDEX and elastomer), or a mixture thereof.

Clause 107. The article of any one of clauses 101-106, wherein the coating further comprises one or more of a wetting agent, an anti-foaming agent, a softener, a wicking agent, and an anti-microbial.

Clause 108. The article of any one of clauses 101-107, wherein the w/w ratio of silk fibroin fragments to surfactant in the coating is about 99:1, about 98:2, about 97:3, about 96:4, about 95:5, about 94:6, about 93:7, about 92:8, about 91:9, about 90:10, about 89:11, about 88:12, about 87:13, about 86:14, about 85:15, about 84:16, about 83:17, about 82:18, about 81:19, about 80:20, about 79:21, about 78:22, about 77:23, about 76:24, about 75:25, about 74:26, about 73:27, about 72:28, about 71:29, about 70:30, about 69:31, about 68:32, about 67:33, about 66:34, about 65:35, about 64:36, about 63:37, about 62:38, about 61:39, about 60:40, about 59:41, about 58:42, about 57:43, about 56:44, about 55:45, about 54:46, about 53:47, about 52:48, about 51:49, about 50:50, about 49:51, about 48:52, about 47:53, about 46:54, about 45:55, about 44:56, about 43:57, about 42:58, about 41:59, about 40:60, about 39:61, about 38:62, about 37:63, about 36:64, about 35:65, about 34:66, about 33:67, about 32:68, about 31:69, about 30:70, about 29:71, about 28:72, about 27:73, about 26:74, about 25:75, about 24:76, about 23:77, about 22:78, about 21:79, about 20:80, about 19:81, about 18:82, about 17:83, about 16:84, about 15:85, about 14:86, about 13:87, about 12:88, about 11:89, about 10:90, about 9:91, about 8:92, about 7:93, about 6:94, about 5:95, about 4:96, about 3:97, about 2:98, or about 1:99.

Clause 109. The article of any one of clauses 101-107, wherein the w/w ratio of silk fibroin fragments to surfactant in the coating is about 1:1.

Clause 110. The article of any one of clauses 101-109, wherein the surfactant is selected from coco glucoside, decyl glucoside, lauryl glucoside, sucrose cocoate, capryl/caprylyl glucoside, and caprylyl/capryl glucoside.

Clause 111. The article of any one of clauses 101-109, wherein the surfactant is selected from capryl/caprylyl glucoside and caprylyl/capryl glucoside.

Clause 112. The article of any one of clauses 101-111, wherein the article has an improved moisture management comparative to a similar article comprising a similar fabric but no coating.

Clause 113. The article of clause 112, wherein moisture management is assessed by a water absorbency test, a vertical wicking test, or a dry rate test.

Clause 114. A method of making a silk fibroin coated fabric, comprising: applying to the fabric a solution comprising a surfactant; applying to the fabric a silk fibroin fragments solution; and drying the fabric.

Clause 115. A method of making a silk fibroin coated fabric, comprising: applying to the fabric a solution comprising a surfactant and silk fibroin fragments; and drying the fabric.

Clause 116. The method of clause 114 or 115, wherein the concentration of the silk fibroin fragments in a solution ranges from 0.01 g/L to about 100 g/L.

Clause 117. The method of any one of clauses 114-116, wherein the concentration of the surfactant in a solution ranges from 0.01 g/L to about 100 g/L.

Clause 118. The method of any one of clauses 114-117, wherein the w/w ratio of silk fibroin fragments to surfactant is about 99:1, about 98:2, about 97:3, about 96:4, about 95:5, about 94:6, about 93:7, about 92:8, about 91:9, about 90:10, about 89:11, about 88:12, about 87:13, about 86:14, about 85:15, about 84:16, about 83:17, about 82:18, about 81:19, about 80:20, about 79:21, about 78:22, about 77:23, about 76:24, about 75:25, about 74:26, about 73:27, about 72:28, about 71:29, about 70:30, about 69:31, about 68:32, about 67:33, about 66:34, about 65:35, about 64:36, about 63:37, about 62:38, about 61:39, about 60:40, about 59:41, about 58:42, about 57:43, about 56:44, about 55:45, about 54:46, about 53:47, about 52:48, about 51:49, about 50:50, about 49:51, about 48:52, about 47:53, about 46:54, about 45:55, about 44:56, about 43:57, about 42:58, about 41:59, about 40:60, about 39:61, about 38:62, about 37:63, about 36:64, about 35:65, about 34:66, about 33:67, about 32:68, about 31:69, about 30:70, about 29:71, about 28:72, about 27:73, about 26:74, about 25:75, about 24:76, about 23:77, about 22:78, about 21:79, about 20:80, about 19:81, about 18:82, about 17:83, about 16:84, about 15:85, about 14:86, about 13:87, about 12:88, about 11:89, about 10:90, about 9:91, about 8:92, about 7:93, about 6:94, about 5:95, about 4:96, about 3:97, about 2:98, or about 1:99.

Clause 119. The method of any one of clauses 114-118, wherein a solution further comprises one or more of a wetting agent, an anti-foaming agent, a softener, a wicking agent, and an anti-microbial.

Clause 120. The method of any one of clauses 114-119, wherein the silk fibroin fragments have an average weight average molecular weight selected from between about 1 kDa and about 5 kDa, from between about 5 kDa and about 10 kDa, from between about 6 kDa and about 17 kDa, from between about 10 kDa and about 15 kDa, from between about 14 kDa and about 30 kDa, from between about 15 kDa and about 20 kDa, from between about 17 kDa and about 39 kDa, from between about 20 kDa and about 25 kDa, from between about 25 kDa and about 30 kDa, from between about 30 kDa and about 35 kDa, from between about 35 kDa and about 40 kDa, from between about 39 kDa and about 54 kDa, from between about 39 kDa and about 80 kDa, from between about 40 kDa and about 45 kDa, from between about 45 kDa and about 50 kDa, from between about 50 kDa and about 55 kDa, from between about 55 kDa and about 60 kDa, from between about 60 kDa and about 100 kDa, or from between about 80 kDa and about 144 kDa, and a polydispersity ranging from 1 to about 5.

Clause 121. The method of any one of clauses 114-120, wherein the silk fibroin fragments have a polydispersity from 1 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, or from about 4.5 to about 5.0.

Clause 122. The method of any one of clauses 114-120, wherein the silk fibroin fragments have a polydispersity from about 1.5 to about 3.0.

Clause 123. The method of any one of clauses 114-120, wherein the silk fibroin fragments comprise one or more of low molecular weight silk fibroin fragments and medium molecular weight silk fibroin fragments.

Clause 124. The method of any one of clauses 114-123, wherein a solution further comprises about 0.01% (w/w) to about 10% (w/w) sericin relative to the silk fibroin fragments.

Clause 125. The method of any one of clauses 114-124, wherein the fabric comprises one or more of polyester, polyamide, polyaramid, polytetrafluoroethylene, polyethylene, polypropylene, polyurethane, silicone, mixtures of polyurethane and polyethyleneglycol, ultrahigh molecular weight polyethylene, high-performance polyethylene, nylon, LYCRA (polyester-polyurethane copolymer, also known as SPANDEX and elastomer), or a mixture thereof.

Clause 126. The method of any one of clauses 114-125, wherein the surfactant is selected from coco glucoside, decyl glucoside, lauryl glucoside, sucrose cocoate, capryl/caprylyl glucoside, and caprylyl/capryl glucoside.

Clause 127. The method of any one of clauses 114-125, wherein the surfactant is selected from capryl/caprylyl glucoside and caprylyl/capryl glucoside.

Clause 128. The method of any one of clauses 114-127, wherein the drying comprises heating.

Clause 129. The method of clause 128, wherein the heating does not substantially modify the coating performance.

Clause 130. The method of any one of clauses 114-129, wherein the pH of a solution is acidic.

Clause 131. The method of any one of clauses 114-129, wherein the pH of a solution is between about 4 and about 4.5.

Clause 132. An article prepared by the method of any one of clauses 114-131.

Clause 133. The article of clause 132, wherein the article has an improved moisture management comparative to a similar article comprising a similar fabric but no coating.

Clause 134. The article of clause 133, wherein moisture management is assessed by a water absorbency test, a vertical wicking test, or a dry rate test.

Clause 201. An article comprising a fabric and a coating, wherein the coating comprises a surfactant and/or emulsifier and silk fibroin fragments having an average weight average molecular weight selected from between about 1 kDa and about 5 kDa, from between about 5 kDa and about 10 kDa, from between about 6 kDa and about 17 kDa, from between about 10 kDa and about 15 kDa, from between about 14 kDa and about 30 kDa, from between about 15 kDa and about 20 kDa, from between about 17 kDa and about 39 kDa, from between about 20 kDa and about 25 kDa, from between about 25 kDa and about 30 kDa, from between about 30 kDa and about 35 kDa, from between about 35 kDa and about 40 kDa, from between about 39 kDa and about 54 kDa, from between about 39 kDa and about 80 kDa, from between about 40 kDa and about 45 kDa, from between about 45 kDa and about 50 kDa, from between about 50 kDa and about 55 kDa, from between about 55 kDa and about 60 kDa, from between about 60 kDa and about 100 kDa, or from between about 80 kDa and about 144 kDa, and a polydispersity ranging from 1 to about 5.

Clause 202. The article of clause 201, wherein the silk fibroin fragments have a polydispersity from 1 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, or from about 4.5 to about 5.0.

Clause 203. The article of clause 201, wherein the silk fibroin fragments have a polydispersity from about 1.5 to about 3.0.

Clause 204. The article of clause 201, wherein the silk fibroin fragments comprise one or more of low molecular weight silk fibroin fragments and medium molecular weight silk fibroin fragments.

Clause 205. The article of any one of clauses 201-204, further comprising about 0.01% (w/w) to about 10% (w/w) sericin relative to the silk fibroin fragments.

Clause 206. The article of any one of clauses 201-205, wherein the fabric comprises one or more of polyester, polyamide, polyaramid, polytetrafluoroethylene, polyethylene, polypropylene, polyurethane, silicone, mixtures of polyurethane and polyethyleneglycol, ultrahigh molecular weight polyethylene, high-performance polyethylene, nylon, LYCRA (polyester-polyurethane copolymer, also known as SPANDEX and elastomer), or a mixture thereof.

Clause 207. The article of any one of clauses 201-206, wherein the coating further comprises one or more of a wetting agent, an anti-foaming agent, a softener, a wicking agent, and an anti-microbial.

Clause 208. The article of any one of clauses 201-207, wherein the w/w ratio of silk fibroin fragments to surfactant and/or emulsifier in the coating is about 99:1, about 98:2, about 97:3, about 96:4, about 95:5, about 94:6, about 93:7, about 92:8, about 91:9, about 90:10, about 89:11, about 88:12, about 87:13, about 86:14, about 85:15, about 84:16, about 83:17, about 82:18, about 81:19, about 80:20, about 79:21, about 78:22, about 77:23, about 76:24, about 75:25, about 74:26, about 73:27, about 72:28, about 71:29, about 70:30, about 69:31, about 68:32, about 67:33, about 66:34, about 65:35, about 64:36, about 63:37, about 62:38, about 61:39, about 60:40, about 59:41, about 58:42, about 57:43, about 56:44, about 55:45, about 54:46, about 53:47, about 52:48, about 51:49, about 50:50, about 49:51, about 48:52, about 47:53, about 46:54, about 45:55, about 44:56, about 43:57, about 42:58, about 41:59, about 40:60, about 39:61, about 38:62, about 37:63, about 36:64, about 35:65, about 34:66, about 33:67, about 32:68, about 31:69, about 30:70, about 29:71, about 28:72, about 27:73, about 26:74, about 25:75, about 24:76, about 23:77, about 22:78, about 21:79, about 20:80, about 19:81, about 18:82, about 17:83, about 16:84, about 15:85, about 14:86, about 13:87, about 12:88, about 11:89, about 10:90, about 9:91, about 8:92, about 7:93, about 6:94, about 5:95, about 4:96, about 3:97, about 2:98, or about 1:99.

Clause 209. The article of any one of clauses 201-207, wherein the w/w ratio of silk fibroin fragments to surfactant and/or emulsifier in the coating is about 1:1, about 1:2, about 1:4, about 1:8, about 1:16, or about 1:32.

Clause 210. The article of any one of clauses 201-209, wherein the emulsifier and/or surfactant is selected from polyoxyethylene sorbitan monooleate, polyoxyethylene sorbitan trioleate, polyoxyethylene castor oil, and any combination thereof.

Clause 211. The article of any one of clauses 201-209, wherein the emulsifier and/or surfactant is selected from polyoxyethylene (10-30) sorbitan monooleate, polyoxyethylene (10-30) sorbitan trioleate, polyoxyethylene (10-50) castor oil, and any combination thereof.

Clause 212. The article of any one of clauses 201-209, wherein the emulsifier and/or surfactant is selected from polyoxyethylene (20) sorbitan monooleate, polyoxyethylene (20) sorbitan trioleate, polyoxyethylene (29) castor oil, and any combination thereof.

Clause 213. The article of any one of clauses 201-212, wherein the emulsifier and/or surfactant has an HLB between 11 and 13.50.

Clause 214. The article of any one of clauses 201-213, wherein the article has an improved moisture management comparative to a similar article comprising a similar fabric but no coating.

Clause 215. The article of clause 214, wherein moisture management is assessed by a water absorbency test, a vertical wicking test, or a dry rate test.

Clause 216. The article of any one of clauses 201-213, wherein the article has an improved drapability comparative to a similar article comprising a similar fabric but no coating.

Clause 217. The article of any one of clauses 201-213, wherein the article has an improved smoothness comparative to a similar article comprising a similar fabric but no coating.

Clause 218. The article of any one of clauses 201-213, wherein the article has an improved hand feel comparative to a similar article comprising a similar fabric but no coating.

Clause 219. A method of making a silk fibroin coated fabric, comprising: applying to the fabric a solution comprising a surfactant and/or emulsifier system; applying to the fabric a silk fibroin fragments solution; and drying the fabric.

Clause 220. A method of making a silk fibroin coated fabric, comprising: applying to the fabric a solution comprising a surfactant and/or emulsifier system and silk fibroin fragments; and drying the fabric.

Clause 221. The method of clause 219 or 220, wherein the concentration of the silk fibroin fragments in a solution ranges from 0.01 g/L to about 100 g/L.

Clause 222. The method of any one of clauses 219-221, wherein the concentration of the surfactant and/or emulsifier system in a solution ranges from 0.01 g/L to about 100 g/L.

Clause 223. The method of any one of clauses 219-222, wherein the w/w ratio of silk fibroin fragments to the surfactant and/or emulsifier system is about 99:1, about 98:2, about 97:3, about 96:4, about 95:5, about 94:6, about 93:7, about 92:8, about 91:9, about 90:10, about 89:11, about 88:12, about 87:13, about 86:14, about 85:15, about 84:16, about 83:17, about 82:18, about 81:19, about 80:20, about 79:21, about 78:22, about 77:23, about 76:24, about 75:25, about 74:26, about 73:27, about 72:28, about 71:29, about 70:30, about 69:31, about 68:32, about 67:33, about 66:34, about 65:35, about 64:36, about 63:37, about 62:38, about 61:39, about 60:40, about 59:41, about 58:42, about 57:43, about 56:44, about 55:45, about 54:46, about 53:47, about 52:48, about 51:49, about 50:50, about 49:51, about 48:52, about 47:53, about 46:54, about 45:55, about 44:56, about 43:57, about 42:58, about 41:59, about 40:60, about 39:61, about 38:62, about 37:63, about 36:64, about 35:65, about 34:66, about 33:67, about 32:68, about 31:69, about 30:70, about 29:71, about 28:72, about 27:73, about 26:74, about 25:75, about 24:76, about 23:77, about 22:78, about 21:79, about 20:80, about 19:81, about 18:82, about 17:83, about 16:84, about 15:85, about 14:86, about 13:87, about 12:88, about 11:89, about 10:90, about 9:91, about 8:92, about 7:93, about 6:94, about 5:95, about 4:96, about 3:97, about 2:98, or about 1:99.

Clause 224. The method of any one of clauses 219-222, wherein the w/w ratio of silk fibroin fragments to surfactant and/or emulsifier system is about 1:1, about 1:2, about 1:4, about 1:8, about 1:16, or about 1:32.

Clause 225. The method of any one of clauses 219-224, wherein the emulsifier and/or surfactant system comprises one or more of polyoxyethylene sorbitan monooleate, polyoxyethylene sorbitan trioleate, polyoxyethylene castor oil, and any combination thereof.

Clause 226. The method of any one of clauses 219-224, wherein the emulsifier and/or surfactant system comprises one or more of polyoxyethylene (10-30) sorbitan monooleate, polyoxyethylene (10-30) sorbitan trioleate, polyoxyethylene (10-50) castor oil, and any combination thereof.

Clause 227. The method of any one of clauses 219-224, wherein the emulsifier and/or surfactant system comprises one or more of polyoxyethylene (20) sorbitan monooleate, polyoxyethylene (20) sorbitan trioleate, polyoxyethylene (29) castor oil, and any combination thereof.

Clause 228. The method of any one of clauses 219-224, wherein the emulsifier and/or surfactant system has an HLB between 11 and 13.50.

Clause 229. The method of any one of clauses 219-228, wherein a solution further comprises one or more of a wetting agent, an anti-foaming agent, a softener, a wicking agent, and an anti-microbial.

Clause 230. The method of any one of clauses 219-229, wherein the silk fibroin fragments have an average weight average molecular weight selected from between about 1 kDa and about 5 kDa, from between about 5 kDa and about 10 kDa, from between about 6 kDa and about 17 kDa, from between about 10 kDa and about 15 kDa, from between about 14 kDa and about 30 kDa, from between about 15 kDa and about 20 kDa, from between about 17 kDa and about 39 kDa, from between about 20 kDa and about 25 kDa, from between about 25 kDa and about 30 kDa, from between about 30 kDa and about 35 kDa, from between about 35 kDa and about 40 kDa, from between about 39 kDa and about 54 kDa, from between about 39 kDa and about 80 kDa, from between about 40 kDa and about 45 kDa, from between about 45 kDa and about 50 kDa, from between about 50 kDa and about 55 kDa, from between about 55 kDa and about 60 kDa, from between about 60 kDa and about 100 kDa, or from between about 80 kDa and about 144 kDa, and a polydispersity ranging from 1 to about 5.

Clause 231. The method of any one of clauses 219-230, wherein the silk fibroin fragments have a polydispersity from 1 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, or from about 4.5 to about 5.0.

Clause 232. The method of any one of clauses 219-230, wherein the silk fibroin fragments have a polydispersity from about 1.5 to about 3.0.

Clause 233. The method of any one of clauses 219-232, wherein the silk fibroin fragments comprise one or more of low molecular weight silk fibroin fragments and medium molecular weight silk fibroin fragments.

Clause 234. The method of any one of clauses 219-233, wherein a solution further comprises about 0.01% (w/w) to about 10% (w/w) sericin relative to the silk fibroin fragments.

Clause 235. The method of any one of clauses 219-234, wherein the fabric comprises one or more of polyester, polyamide, polyaramid, polytetrafluoroethylene, polyethylene, polypropylene, polyurethane, silicone, mixtures of polyurethane and polyethyleneglycol, ultrahigh molecular weight polyethylene, high-performance polyethylene, nylon, LYCRA (polyester-polyurethane copolymer, also known as SPANDEX and elastomer), or a mixture thereof.

Clause 236. The method of any one of clauses 219-235, wherein the drying comprises heating.

Clause 237. The method of any one of clauses 219-236, wherein the pH of a solution is acidic.

Clause 238. The method of any one of clauses 219-236, wherein the pH of a solution is between about 4 and about 4.5.

Clause 239. An article prepared by the method of any one of clauses 219-238.

Clause 240. The article of clause 239, wherein the article has an improved moisture management comparative to a similar article comprising a similar fabric but no coating.

Clause 241. The article of clause 240, wherein moisture management is assessed by a water absorbency test, a vertical wicking test, or a dry rate test.

Clause 242. The article of clause 239, wherein the article has an improved drapability comparative to a similar article comprising a similar fabric but no coating.

Clause 243. The article of clause 239, wherein the article has an improved smoothness comparative to a similar article comprising a similar fabric but no coating.

Clause 244. The article of clause 239, wherein the article has an improved hand feel comparative to a similar article comprising a similar fabric but no coating.

### EXAMPLES

The following examples are put forth so as to provide those of ordinary skill in the art with a complete disclosure and description of how to make and use the described embodiments, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1: Scaleup Methods

In some embodiments, a scaleup method comprises a lower gradient of temperature (Δ°) between any two points, two planes, or two voxels in a scaleup setup, e.g., and without limitation, a scaleup reaction vessel, compared to any similarly situated two points, two planes, or two voxels in a bench setup, e.g., and without limitation, a bench reaction vessel. In some embodiments, the scaleup gradient is lower than the bench gradient by about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., or about 20° C.

Extraction:

| | Bench | | Scaleup | |
|---|---|---|---|---|
| Cocoon mass | 95.3 | g | 3750 | g |
| Ext water vol | 8 L (RODI) | | 88.2 L (city) | |
| Sodium carbonate mass | 21.2 | g | 834.2 | g |
| Ext water temp | 100 | C. | 100 | C. |
| Extraction time | 60 | min | 30 | min |
| Rinse water vol | 8 L (RODI) | | 88.2 L (city) | |
| Rinse water temp | 60 | C. | 60 | C. |
| Rinse time | 20 | min | 15 | min |

| | | |
|---|---|---|
| Number of rinses | 3 | 3 |
| Notes | Intermittent | Intermittent |
| | agitation done by | agitation done by |
| | hand. Silk is wrung | washer drum |
| | by hand to remove | rotation. Water |
| | extraction and rinse | removal done by |
| | water between steps | spin cycle after |
| | | final rinse (no spin |
| | | cycles between |
| | | washes/rinses) |

Dissolution:

| | Bench (mid) | | Bench (low) | | Scaleup (mid) | | Scaleup (low) | |
|---|---|---|---|---|---|---|---|---|
| Fibroin mass | 100 | g | 100 | g | 10375 | g | 10375 | g |
| 9.3M LiBr vol | 400 | mL | 400 | mL | 41.5 | L | 41.5 | L |
| Dissolution temp | 100 | C. | 134 | C. | 103 | C. | 125 | C. |
| Combination time | 1-3 | minutes | 1-3 | minutes | 30-60 | min | 30-60 | min |
| Reaction time | 60 | min | 360 | min | 60 | min | 360 | min |

| | | | | |
|---|---|---|---|---|
| Notes | Reaction done in 5 | Reaction done in 5 | Reaction done in 50 L jacketed | Reaction done in 50 L jacketed |
| | L beaker fully | L beaker fully | glass reactor. Combination | glass reactor. Combination |
| | contained in an | contained in an | carried out by repetitively | carried out by repetitively |
| | oven. Beaker is | oven. Beaker is | submerging and agitating | submerging and agitating fibroin |
| | mostly unagitated | mostly unagitated | fibroin until all fibroin is | until all fibroin is added. Mixture |
| | after initial | after initial | added. Mixture is agitated at | is agitated at 25 RPM for duration |
| | combination | combination | 25 RPM for duration of | of reaction time. |
| | | | reaction time. | |

### Example 2: Derivatized or Modified Peptides

A novel method is disclosed to generate compositions of polypeptides that are derived from B. mori silkworm cocoons and comprised of natural and modified polypeptides. These two novel compositions are called Low Skid and Mid Skid silk/modified polypeptide compositions.

The novel production method involves removing sericin through several washing steps with an organic sodium carbonate salt with tightly controlled multi-stage temperature cycles and agitation as the first step in forming natural/modified polypeptide composition. Next the silk is dried to remove remaining water at controlled temperature to maintain polypeptide composition. The silk is then dissolved in high concentration of Lithium salt at two different temperatures and times to achieve the different compositions for Mid and Low silk. The liquid solution is then filtered and purified to remove the Lithium salt leaving only the natural/modified silk compositions in solution with pure water.

Low Skid and Mid Skid silk/modified polypeptide compositions comprise of populations of silk/modified polypeptides with distinctive properties. Low Skid silk/modified polypeptide composition does not self-assemble at 5 mg/mL. Low Skid silk/modified polypeptide composition comprises of two main populations of silk/modified polypeptides; one population (AS22) that does not self-assemble under conditions that promote self-assembly at 5 mg/mL and is rich in negatively charged amino acids; a second population of polypeptides (AS12) that self-assemble fast at 5 mg/mL and are depleted on negatively charged amino acids.

When AS12 and AS22 are combined at a ratio of 50% each the average molecular weight of the mixture becomes the same as Low Skid silk/modified polypeptide composition. Thus Low Skid silk/modified polypeptide composition consists of 50% AS12 and 50% AS22 silk/modified polypeptide compositions.

Mid Skid silk/modified polypeptide composition comprises of two main populations of silk/modified polypeptides; one population (AS11) that self-assemble slower than Mid Skid silk/modified polypeptides, under conditions that promote self-assembly at 5 mg/mL and is rich in negatively charged amino acids; a second population of polypeptides (AS1) that self-assemble faster than Mid Skid silk/modified polypeptides, under conditions that promote self-assembly at 5 mg/mL and are depleted on negatively charged amino acids.

When AS11 and AS11 are combined at a ratio of 50% each the average molecular weight of the mixture becomes the same as Mid Skid silk/modified polypeptide composition. Thus, Low Skid silk/modified polypeptide composition consists of 50% AS1 and 50% AS11 silk/modified polypeptide compositions.

Both Low Skid and Mid Skid silk/modified polypeptide compositions contain modified peptides that were determined after analysis with Mass Spectrometry.

Low and Mid Skid silk/modified polypeptide compositions described in this disclosure are never produced before compositions of silk-derived and modified polypeptides that when isolated display a wide range of behaviors, from extreme self-assembly to solubility and stability over time in various buffers and various average molecular weights and polydispersities. These novel silk-derived polypeptide compositions contain unique modified amino acid sequences that result from a unique silk processing method and scaleup. The tight controls around temperature, silk concentration, salt concentrations, physical agitation and purification allow us to tune at each step of the process the unique peptide compositions in the natural/modified silk species to design for specific performance criteria. Some of constituent polypeptide compositions display biological activity and could be used as therapeutic candidates.

Silk is a versatile material that can be used in many applications from development of implantable medical devices to the development of soluble polypeptide formulations of medicinal value. A major challenge with silk polypeptides in solution is their tendency to self-assemble and aggregate, making the control of their solubility very difficult. Also, the kinetics of gel/film formation cannot be controlled in a predictable way. This novel silk/modified peptide compositions contain populations of peptides that allows to control their properties and develop products with predictable and desired properties.

The disclosed compositions contain a collection of many polypeptides with different properties. Silk has been characterized mostly based on its molecular weight and polydispersity, and no mixture of silk/modified polypeptides has been characterized or has been generated. As disclosed herein, a unique large scale process is used to generate compositions of silk/modified polypeptides.

Low/Mid skid silks begin their process to remove sericin using sodium carbonate at specific silk mass, sodium carbonate, and water ratios. Multiple different temperature washing cycles 100° C. and 60° C. and agitation is also key in producing the specific natural/modified compositions. The silk is then dried to remove water at a specific temperature that maintains the silk composition. Next the silk is dissolved in a high concentration of Lithium Bromide at 103° C. and 125° C. for 1 and 6 hours respectively. The time and temperature allow for fine tuning the degree of post translational modifications that give the unique polypeptide compositions. The silk is then purified to remove Lithium Bromide and optionally concentrate the silk. For the downstream characterization of isolation of the various silk/modified polypeptide compositions chromatographic techniques were employed, biochemical/biophysical techniques, and cell biology methods.

To characterize/separate novel Low and Mid Skid silk/modified polypeptide compositions a combinations of Ion Exchange Chromatography fractionation, analytical methods, and biochemical dissection were used to characterize its properties.

### Generation of Low and Mid Skid Silk/Modified Polypeptide Compositions.

Silk is washed to remove sericin at 100° C. and 60° C. with sodium carbonate and then dried at 60° C. The silk is then dissolved in 9.3 M Lithium Bromide at 103° C. for 1 hour for Mid silk and 9.3 M Lithium Bromide at 125° C. for 6 hours for Low silk. This dissolution step controls not only molecular weight but also the polypeptide modifications creating the natural/modified silk compositions. The silk is then filtered to remove undissolved debris and purified using 10 KDa cutoff PES hollow fiber membranes, and concentrated using the same process leaving only natural/modified silk composite in solution with pure water. Every unit ops is tightly controlled for temperature, time, concentrations, agitation, and shear.

### Isolation of Low and Mid Skid/Modified Polypeptide Compositions

### Isolation of the AS22 Silk/Modified Polypeptide Composition Component of Low Skid Silk/Modified Polypeptide Composition.

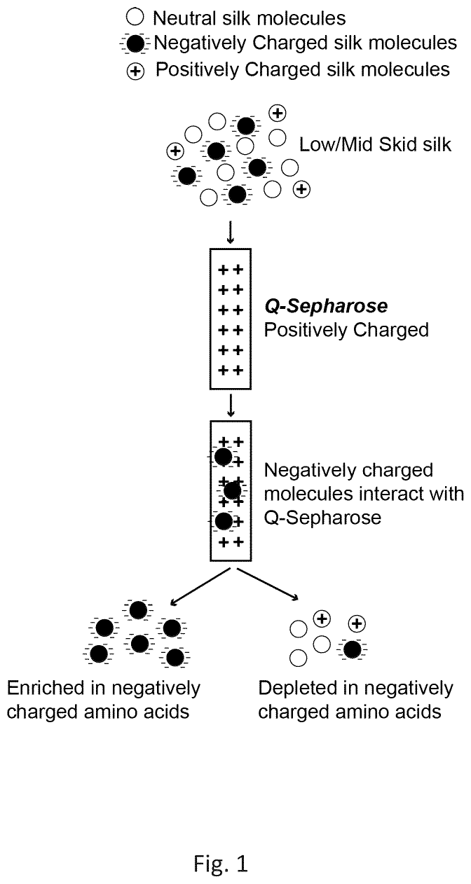

To isolate AS22 it was fractionated Low Skid silk/modified polypeptide compositions using HiTrap Q Sepharose Anion Exchanger (FIGS. 1,2). Tris was added in the silk preparations to a final concentration of 50 mM Tris-HCl pH=8.0. Silk was centrifuged before loading the HiTrap Q Sepharose column to remove any preformed aggregates. Low Skid silk preparation solutions have a characteristic yellow hue. The flow through from the HiTrap Q Sepharose column was transparent. AS22 eluted with 1M NaCl and it has an intense yellow hue.

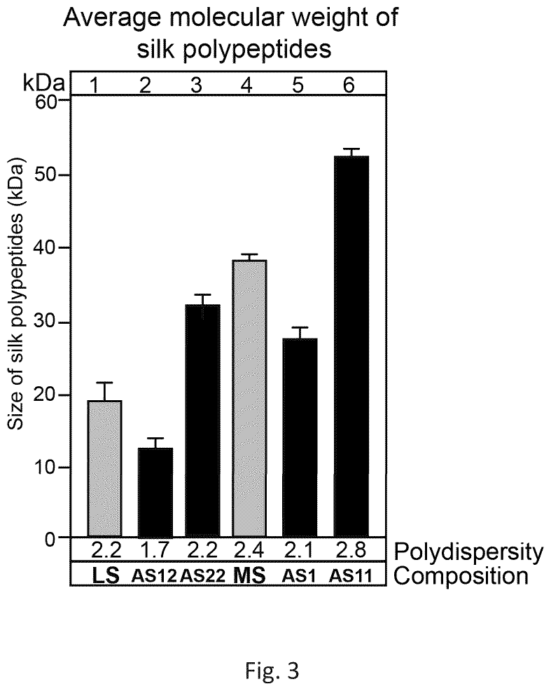

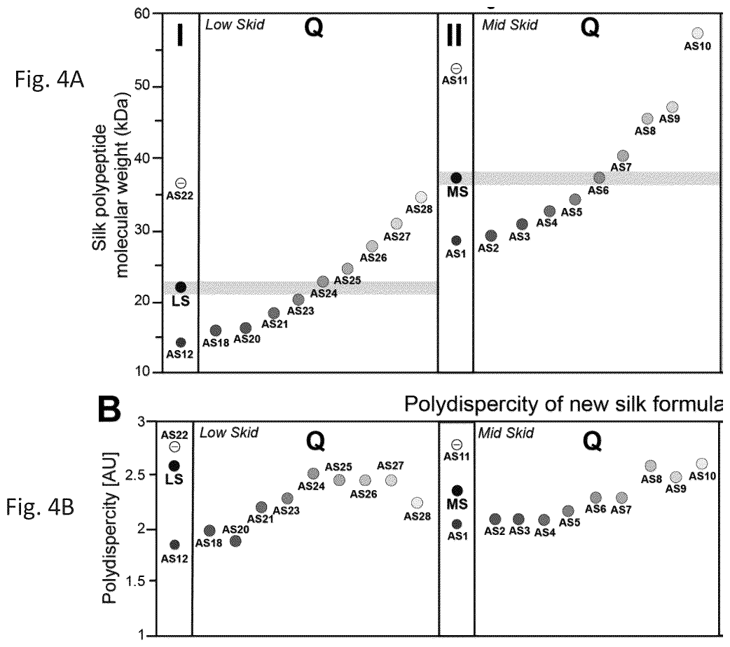

When AS22 formulation is analyzed with an analytical SEC column (see materials and methods) with HPLC it has an average molecular weight (M_{w}) of about 35 kDa and Polydispersity (PDI) of 2.3 (FIGS. 3, 4). This molecular weight and polydispersity are distinctive and different from Low Skid silk/modified polypeptide composition (MW about 19.5 kDa and PDI 2.2) and Mid Skid silk/modified polypeptide composition (MW about 38 kDa and PDI 2.4) (FIGS. 3, 4).

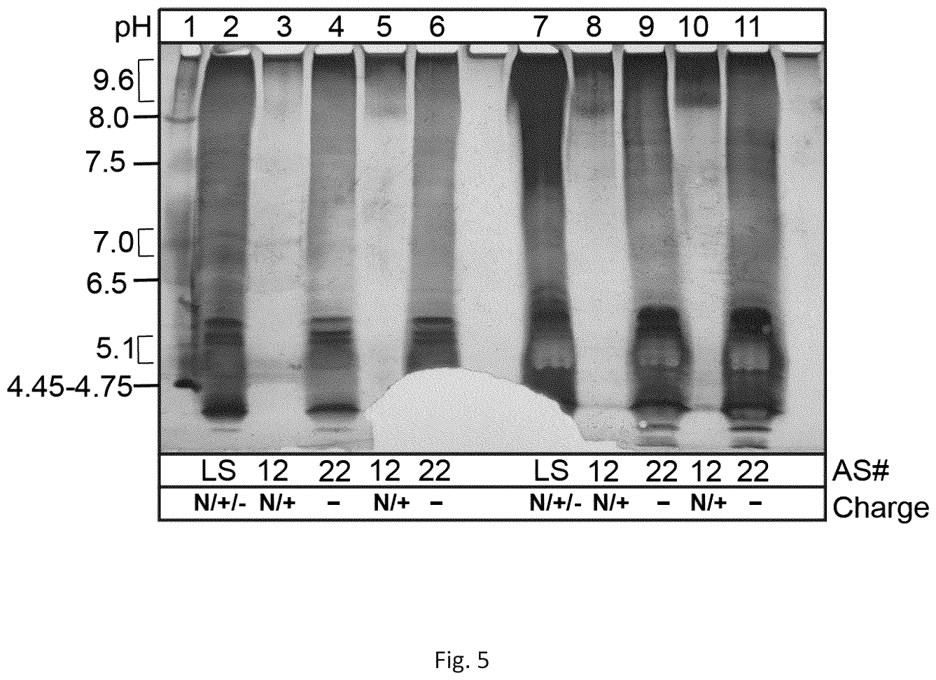

When AS22 and Low Skid silk were analyzed in Isoelectric Focusing Polyacrylamide gels, it was found that 22M silk peptides had pIs 3-6 and some species with pIs of about 9.6, whereas Low Skid silk also contains peptides that span the whole range of pIs, from 3 to 9.6 (FIG. 5).

### Isolation of the AS12 Silk/Modified Polypeptide Composition Component of Low Skid Silk/Modified Polypeptide Composition.

To develop AS12 a combination of Ion Exchange Chromatography (IEX) fractionation was used to purify the formulation, analytical methods and biochemical dissection to characterize its properties.

To isolate AS12 Low Skid silk preparations was fractioned using HiTrap Q Sepharose Anion Exchanger (FIG. 1,2). Tris was added in the silk preparations to a final concentration of 50 mM Tris-HCl pH=8.0. Silk was centrifuged before loading the HiTrap Q Sepharose column to remove any preformed aggregates. The flow through from the HiTrap Q Sepharose column was collected and was designated AS12. AS12 is colorless.

### AS12 Silk Formulation is Composed of Short Silk/Modified Polypeptides Depleted in Negative Charges.

When AS12 was analyzed with analytical Size Exclusion Chromatography, it has an average molecular weight (M_{w}) of about 12 kDa and Polydispersity (PDI) of 1.7 (FIG. 3,4). This molecular weight and polydispersity are distinctive and different from Low Skid silk/modified peptide composition (MW about 19.5 kDa and PDI 2.2) and Mid Skid silk/modified peptide composition (MW about 38 kDa and PDI 2.4) (FIG. 3,4). When AS12 and Low Skid silk/modified peptide composition were analyzed in Isoelectric Focusing Polyacrylamide gels, it was found that AS12 silk peptides had a pI of about 9-10, whereas Low Skid silk also contains peptides that span the whole range of pIs, from 3 to 9.6 (FIG. 5) with most of them concentrated around pI 3-5.5.

### Isolation of the AS) Silk/Modified Polypeptide Composition Component of Mid Skid Silk/Modified Polypeptide Composition.

To isolate AS1 Low Skid silk preparations were fractioned using HiTrap Q Sepharose Anion Exchanger (FIG. 1, 2). Tris was added in the silk preparations to a final concentration of 50 mM Tris-HCl pH=8.0. Silk was centrifuged before loading the HiTrap Q Sepharose column to remove any preformed aggregates. The flow through from the HiTrap Q Sepharose column was collected and was designated AS1. AS1 has a MW of about 28 kDa and PDI of 1.7-2.1 (FIG. 3,4). Mid Skid silk/modified peptide composition has MW of 37 kDa PDI 2.0 (FIG. 3,4).

### Isolation of the AS11 Silk/Modified Polypeptide Composition Component of Mid Skid Silk/Modified Polypeptide Composition.

To develop AS11 a combination of Ion Exchange Chromatography (IEX) fractionation to purify the formulation, analytical methods and biochemical dissection to characterize its properties was used. To isolate AS11 Mid Skid silk/modified peptide composition preparations was fractionated using HiTrap Q Sepharose Anion Exchanger (FIG. 1, 2). Tris was added in the silk preparations to a final concentration of 50 mM Tris-HCl pH=8.0. Silk was centrifuged before loading the HiTrap Q Sepharose column to remove any preformed aggregates. The elution from the HiTrap Q Sepharose column was collected and was designated AS11. AS11 formulation has a molecular weight (M_{w}) of about 53 kDa and Polydispersity (PDI) of 2.8 (FIG. 3, 4). This molecular weight and polydispersity are distinctive and different from Mid Skid silk/modified peptide composition (MW, about 37 kDa and PDI 2.4) (FIG. 3, 4).

### Self-Assembly of Low and Mid Skid/Modified Polypeptide Compositions

Self Assembly Assay and Data Derived from It. To study the stability of AS1 in solution self-assembly assays was performed.

AS1 silk at 5 mg/mL self assembles very fast. The absorbance at 550 nm curves of the self-assembly assays are sigmoid and they can be described as logistic curves. The typical logistic function is:

fx=Amax 1+e−k(t−t0.5)
Aₘₐₓ is the maximum density of the gel formed
k is the Self Assembly Rate Factor
t_{0.5} is the time point at which 50% of the gel has formed
e is the exponential equation for the specific curve
(see FIG. 8 A the red dotted lines for a better demonstration of how these factors were calculated from the self-assembly experiments)

Another parameter introduced to characterize the propensity of silk to form gels is the Self Assembly Factor which is:

$${f}_{SAF} = 1 {t}_{0.5} \times {A}_{max} \times 1000$$

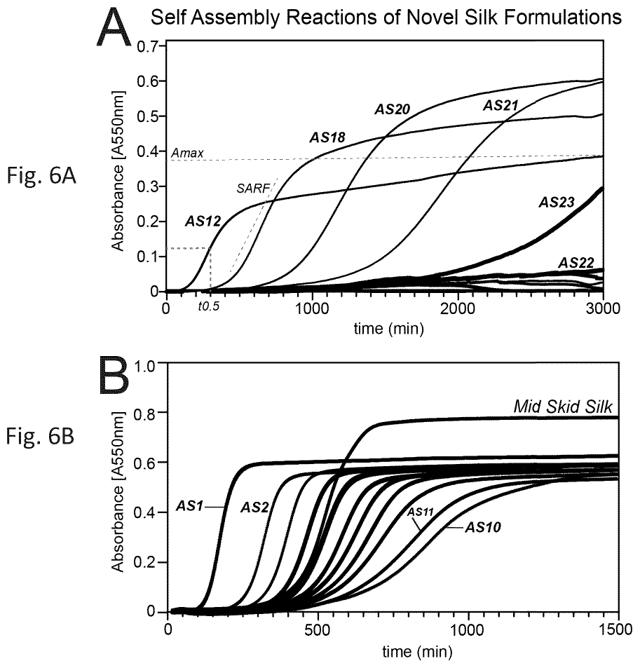

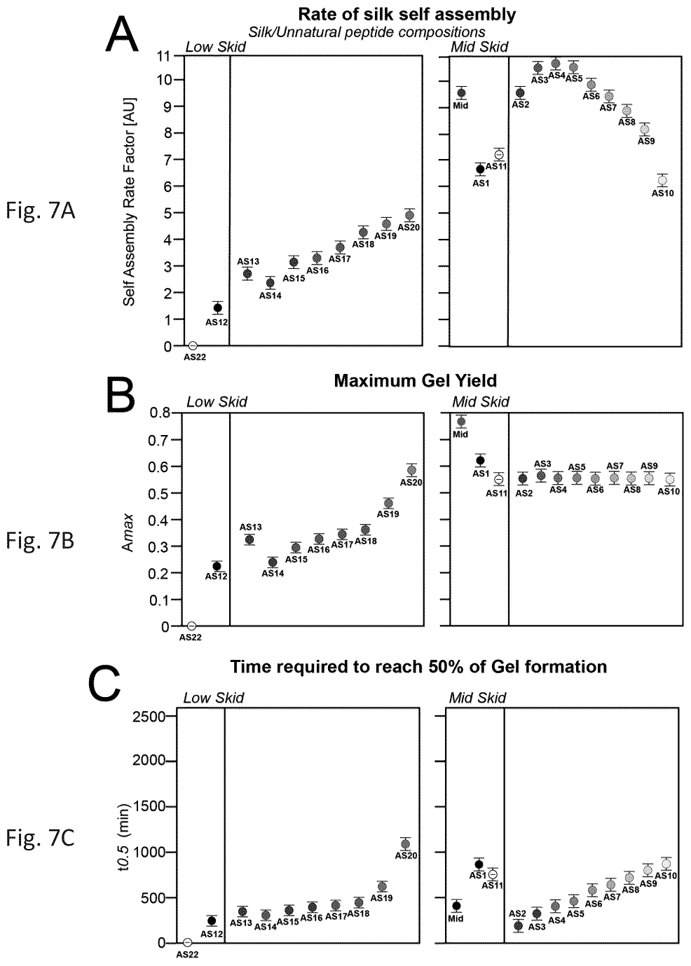

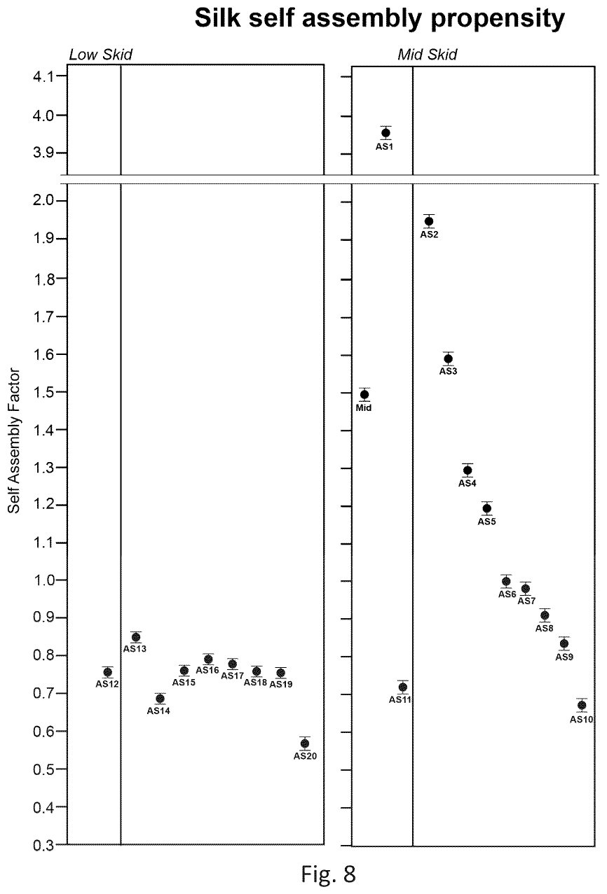

Using the experimental data from the self-assemble assays performed with the various novel isolated silk polypeptides these parameters calculated and use to dissect their properties (FIG. 6, 7, 8). Four parameters were in focus, collectively referred to as Self-assembly kinetic factors; the Self-Assembly Rate Factor (SARF), Aₘₐₓ t0.5, and the Self Assembly Factor (SAF) (FIG. 6, 7, 8). The SARF shows how fast silk self-assembles to form gel after the reaction begins or the gelation nuclei have formed; Aₘₐₓ shows how dense is the gel that is formed after self-assembly is complete, t0.5 shows how long it takes for the self-assembly reaction to reach the point where gel density is Amax2 and SAF shows the propensity of silk to self-assemble (FIG. 6, 7, 8).

### AS1 Silk/Modified Polypeptide Composition has the Fastest Self-Assembly Kinetics.

Self-assembly assays as described before, revealed that AS1 self-assembles very fast, much faster than Mid Skid silk/modified peptide compositions (FIG. 6, 7, 8). Mid Skid silk/modified peptide compositions was used as a positive control and shows fast self-assembly kinetics (FIG. 6, 7, 8).

### AS11 Silk/Modified Polypeptide Composition has the Fastest Self-Assembly Kinetics.

Self-assembly assays as described before, revealed that AS11 self-assembles fast but not as fast as Mid Skid silk/modified peptide compositions (FIG. 6, 7, 8. Mid Skid silk/modified peptide compositions was used as a positive control and shows fast self-assembly kinetics (FIG. 6, 7, 8).

AS12 Silk/Modified Polypeptide Composition is the Component of Low Skid Silk/Modified Polypeptide Composition that Promotes Self-Assembly.

Self-assembly assays as described before, revealed that AS12 self-assembles fast but not as fast as Mid Skid silk/modified peptide compositions (FIG. 6, 7, 8. Mid Skid silk/modified peptide compositions was used as a positive control and shows fast self-assembly kinetics (FIG. 6, 7, 8).

### AS22 Silk/Modified Polypeptide Composition is Very Stable in Aqueous Solution and Doesn't Self-Assemble.

Self-assembly assays as described before, revealed that AS22 displays a remarkable stability and doesn't self-assemble even in conditions that promote silk self-assembly. Mid Skid silk was used as a positive control and shows fast self-assembly kinetics (FIG. 6, 7, 8).

When AS1, AS11, AS12 and AS22 Silk/Modified Polypeptide Compositions are Combined at Different Ratios they Result in Compositions with Unique Properties.

To better understand the properties of the silk/modified polypeptide compositions (FIG. 6, 7, 8) a series of mixtures were created (see table 1). The resulting compositions display a unique combination of properties in self-assembly assays (FIG. 6, 7, 8). Mixtures of AS1 and AS11 (AS2-10) and AS12 with AS22 (AS13-AS21) all display a unique combination of self-assembly kinetics (FIG. 6, 7, 8). This information can be used to create silk/modified polypeptide compositions with specific desired properties.

### Materials and Methods Used for the Generation and Characterization of AS1-AS28.

### Anion Exchange Fractionation of Silk.

Low Skid silk was provided at a concentration of 60 mg/mL. Low Skid silk was centrifuged at 20,000×g, at 4° C. for 15 min to remove any aggregated material. For the silk fractionation Q-Sepharose prepacked columns connected to an AKTA pure 25 L (Cytiva, Serial Number 2747908) or columns packed with Q-Big Beads resin (Cytiva) were used. All buffers used were filtered through a 0.45 μm PES filter and degassed with sonication. Centrifuged Low or Mid Skid silk was loaded on 5×5 mL HiTrap Q HP columns (Cytiva 17115401, Lot:10305274) washed with 10 column volumes of 50 mM Tris pH=8.0, 10 column volumes of 50 mM Tris pH=8.0 (Tris Base, Goldbio CAT #T-400-5, CAS #77-86-1, Lot #250501T400), 1M NaCl (NaCl, VWR High Purity Grade, X190-5 kg, Lot #20G2756431) and finally 10 column volumes of 50 mM Tris pH=8.0, 100 mL of centrifuged Low Skid silk were loaded on the column with a flow rate of 5 mL/min. The flow-through was collected. The column was washed with 50 mM Tris pH=8.0 until the absorbance at 280 nm [A₂₈₀] got to 100 AU. Bound protein was eluted in one step with 50 mM Tris pH=8.0, 1M NaCl and all fractions with absorbance [A₂₈₀]>500AU were pooled together. 20 mL of the Low Skid silk (LS) that was used for the fractionation, the flow through (QFT) and the pooled elution fractions (QE) were placed in dialysis bags (Sigma D9652-100FT, Dialysis tubing cellulose membrane avg flat width 33 mm, 1.3 in., PCode1003241246, Lot #3110) and were immersed in 50× volumes of 50 mM Tris pH=8.0. Samples were flash frozen in liquid nitrogen and stored at −20° C. until they were used. Before use samples were thawed slowly at 4° C. or on ice.

### Analytical/Protein Characterization Methods.

### Protein Concentration Determination.

Protein concentration was determined by absorbance at 220 nm or 280 nm.

Solubilized silk preparations were diluted until A₂₈₀ was between 0.1-1. In this range the absorbance correlates linearly with the concentration of silk in the solution and the correlation is 1AU=1 mg/mL soluble silk proteins. Final concentrations in the initial silk solution were calculated after adjustment for the dilution used for the absorbance measurement.

### Analytical Size Exclusion Chromatography.

Analysis was performed in a PolySep GFC P-4000 LC Column, 300 mm×7.8 mm (Phenomenex, Part No. CHO-9229) connected to a Agilent 1260 Infinity II HPLC system with an Agilent G7162A RID Refractive Index Detector. The mobile phase used for the analysis was a solution of 0.1M NaCl, 12.5 mM Na₂HPO₄, pH 7 (the pH was adjusted with phosphoric acid and filtered through a 0.2 μm PES filter into a clean glass media bottle). 25 μL of sample were loaded on the column and the analysis was performed at 25° C. with a flow rate of 1 mL/min for 20 min. Calculation of the molecular weight of each sample was done using Agilent Technologies Open LAB CDS ChemStation Edition for LC & LC/MS Systems software Cirrus SEC data collection and molecular weight analysis software. (For more details see Report XXX).

### Analytical Anion Exchange Chromatography.

For the analysis an HPLC Dionex Ultimate 3000 and a TSKgel (07163) DEAE-3SW column 7.5 mm ID×7.5 cm, 10 μm was used. Chromatography was performed at 25° C. 10% Trifluoroethanol, 45% Acetonitrile in water was used as a blank solution. The column was equilibrated with 20 mM Na₂HPO₄/KH₂PO₄ (Na₂HPO₄: Fisher Chemical, Lot #188298, PN #S374-500, KH₂PO₄: Fisher Chemical, Lot #187270, PN #P285-500), pH 6 at 1 mL/min for 10 min. 30 μL of sample were loaded on the column. Bound silk polypeptides were eluted with a linear gradient of 500 mM NaCl (NaCl: Fisher Chemical, Lot #206719, PN #BP358-1). All solutions were made in LCMS water: Fisher Chemical, Lot #216650, PN #W5-4. Collected data were analyzed with XCaliburTi Software.

### Isoelectric Point Determination of Silk.

To determine the isoelectric point of the new silk compositions Isoelectric Focusing gels were used. These separate proteins based on their net charge and not their molecular weight. For the analysis, a BIO-RAD Criterion Precast Gels was used, IEF standards pI 4.45-9.6 (BIO-RAD, Cat #1610310, Batch 64417452, L0040178). Silk protein samples from new silk compositions were mixed with IEF Sample Buffer (BIO-RAD Cat #16110763, Batch 64345676, L004138B) (make sure that you have at least 5% v/v glycerol in the final mix). The mixtures were loaded on a Criterion Precast Gel. For the electrophoresis a 1×IEF Anode Buffer (BIO-RAD Cat #1610761, Batch 64376614) and 1×IEF Cathode Buffer (BIO-RAD, Cat #1610762, Batch 64364827) were used. The running conditions for the electrophoresis were: 100V constant for 60 min, 250V constant for 60 min and 500V constant for 30 min.

After the electrophoresis was complete proteins were fixed on the gel with a solution of 40% v/v Methanol (Sigma Aldrich, Methanol ACS reagent>99.8%, 179337-4L-Pb,Source SHBN0806, Pcode 1003210445), 10% v/v Acetic acid (VWR Acetic Acid, Glacial, ACS Grade, BDH3092-500MLP, Lot: 2018071399), for 30 min to overnight at room temperature with rocking.

### LC/MS Analysis of Polypeptides.

Samples were stored at 4° C. until used for analysis. For each sample, an aliquot was taken and mixed with an equal volume of 6 M guanidine hydrochloride (GuHCl) in a new tube. From that mix, an aliquot was taken again to create 120-fold and 240-fold further dilutions for determining protein concentrations using the BCA assay. Using the concentrations determined above, samples were diluted to 20 μg/μL with 6 M GuHCl. An aliquot of 1,000 μg total protein was transferred to a new tube. 50 mM dithiothreitol (DTT) was added to a final concentration of 5 mM and the samples were incubated at 60° C. for 30 minutes. After a brief equilibration period to room temperature, 100 mM iodoacetamide (IAM) was added to a final concentration of 10 mM and the samples were incubated at room temperature in the dark for 30 minutes. The IAM reaction was quenched by the addition of 50 mM DTT to a final concentration of 5 mM DTT, followed by a further incubation at room temperature for 30 minutes. Three aliquots corresponding to 100 μg of total protein were taken in separate tubes and diluted in PBS to get a final concentration of 0.2M GuHCl. Samples were then treated with enzyme at a protease to protein ratio of 1:50 (2 μg of each protease) overnight at either room temperature (chymotrypsin) or 37° C. (trypsin/Lys-C and Glu-C). The protease reactions were quenched by the addition of TFA to a final concentration of 1% (v/v). Samples were centrifuged for 10 minutes at 14,000 rpm and supernatant was transferred to HPLC autosampler vials for LC-MS analysis. LC
Column: C18 column (100 μm×150 mm, 3 μm)
Mobile Phase A: Water 0.1% formic acid
Mobile Phase B: Acetonitrile 0.1% formic acid
Flow Rate: 600 nL/min (micro pump) and 15 μL/min (loading pump)
Chromatographic time: 60 minutes
Elution Gradient: 7% B (0 to 3.3 min); 7% to 35% B (3.3 to 35 min); 35% to 95% B (35-37 min); 95% to 80% B (37-39 min); 80% B (39-41 min); 80% to 7% B (41 to 44 min); 7% B (44 to 60 min)
Injection volume: 4 μL

Acquisition for full MS ranges from 350 to 1600m/z. The MS method is based on data-dependent acquisition (DDA) for the top 10 ions with an isolation window of 3.0m/z and a normalized collision energy of 26.

Data was acquired using Thermo Xcalibur™ Software. Data analysis was performed using Thermo Proteome Discoverer™ Software. To unequivocally assign a specific protein from the identified peptides, a minimum of 2 unique peptides per protein are required upon searching against SwissProt database.

### Gel Staining Methods.

### Silver Staining

SDS and IEF polyacrylamide gels were stained using ProteoSilver Silver Stain Kit (Sigma, PROTSIL-1-1KT, Lot #SLCH2293, Pcode: 1003135372) following the manufacturer's instructions. Briefly, gels were immersed in fixing solution (50% v/v Ethanol, 10% Glacial Acetic Acid), washed with water, sensitizer solution, silver solution and developer solution. Gel band development was terminated with ProteoSilver Stop Solution.

### Self Assembly Assay.

The silk Self Assembly Assay (SAF) was performed in 35% v/v 2-propanol (Sigma-Aldrich 2-Propanol ACS reagent>99.5%. 190764-4L, Lot #SHBK7164, PCode 1002789344) and 50 mM CH₃COONa pH=5 (Sodium acetate anhydrous, VWR life sciences Product number 0602-1Kg, Lot #0677C055). Each reaction was done in a final volume of 200 μL. Total silk protein concentration was 5 mg/mL. First the buffer of 50 mM CH₃COONa pH=5, 35% v/v 2-propanol was prepared. Then DI/RO water was added so that after the addition of the volume of silk protein required to reach a final concentration of 5 mg/mL the total volume would be 200 μL. The protein was added last and mixed with very gentle pipetting to reduce shearing force. The protein mixtures were placed in wells of flat-bottom 96-well plates and a layer of 100 μL of Mineral Oil (Sigma, Mineral Oil BioReagent, for molecular biology, light oil, M5904-500 mL, Lot #MKCC7596, PCode 1002883254) carefully so as to not create any bubbles. Absorbance was recorded at 550 nm for 16-24h (depending on the sample). Recorded values were exported in Excel files for storage and further analysis.

### Data Analysis.

Data were analyzed using GraphPad Prism 9 for macOS Version 9.2.0 (283), Jul. 15, 2021. Figures were prepared with Adobe illustrator 25.4.1.

TABLE 1
Detailed composition of all AS products generated herein.
The composition of each product is given in % per mass (mg/mL).
(*use the AS number to locate more information about the
preparations in the AS Library inventory in the ELN)

| | Low | Low | Mid | Mid |
|---|---|---|---|---|
| AS | Skid Q- | Skid Q Flow | Skid Q- | Skid Q Flow |
| number | Elution (%) | Through (%) | Elution (%) | Through (%) |
| 1 | 0 | 0 | 0 | 100 |
| 2 | 0 | 0 | 90 | 10 |
| 3 | 0 | 0 | 80 | 20 |
| 4 | 0 | 0 | 70 | 30 |
| 5 | 0 | 0 | 60 | 40 |
| 6 | 0 | 0 | 50 | 50 |
| 7 | 0 | 0 | 40 | 60 |
| 8 | 0 | 0 | 30 | 70 |
| 9 | 0 | 0 | 20 | 80 |
| 10 | 0 | 0 | 10 | 90 |
| 11 | 0 | 0 | 100 | 0 |
| 12 | 0 | 100 | 0 | 0 |
| 13 | 5 | 95 | 0 | 0 |
| 14 | 2 | 90 | 0 | 0 |
| 15 | 5 | 90 | 0 | 0 |
| 16 | 6 | 90 | 0 | 0 |
| 17 | 8 | 90 | 0 | 0 |
| 18 | 10 | 90 | 0 | 0 |
| 19 | 15 | 85 | 0 | 0 |
| 20 | 20 | 80 | 0 | 0 |
| 21 | 30 | 70 | 0 | 0 |
| 22 | 100 | 0 | 0 | 0 |
| 23 | 40 | 60 | 0 | 0 |
| 24 | 50 | 50 | 0 | 0 |
| 25 | 60 | 40 | 0 | 0 |
| 26 | 70 | 30 | 0 | 0 |
| 27 | 80 | 20 | 0 | 0 |
| 28 | 90 | 10 | 0 | 0 |

### Example 3. Skid Manufacturing Process Examples for Specific New Low, Mid & High Silk Fragment Populations Each Having a Single Weight Average Molecular Weight Average (MW) with Polydispersity Less than 5 and/or Less than 3

### Fibroin Isolation

Fibroin isolation requires separation of sericin from raw B. mori silk fibers and cocoons. This separation is typically carried out in a single stage and facilitated in a solid substrate extraction operation. The unit consists of a vessel vented to atmosphere and encloses a perforated drum which rotates on a horizontal axis. Any vessel or drum type, vented to atmosphere or operated under pressure, may be used so long as the controls and gradients described are achieved with or without agitation in the reactor or mixing of the reaction components on any axis of rotation.

The raw silk fibers are introduced into the rotating drum via a sealable access port. Raw silk may be added to the vessel or enclosed in a secondary container, such as silk cocoons packed loosely into permeable mesh bags. This secondary containment step minimizes product loss, protects the equipment by preventing silk strands from entangling with rotating components in the vessel, and protects the drain lines from plugging with solids that would otherwise escape from the drum during processing. The reaction vessel fills with extraction solvent to partially submerge the perforated drum. The reaction extraction solvent is composed of 0.7%-0.95% by weight sodium carbonate in water to partially submerge the perforated drum. Example concentrations of extraction solvent are 0.94% by weight for fragment populations below 40 kDa and 0.70% by weight for fragments below 96 kDa as measured by HPLC with size exclusion chromatography with a refractive index detector (SEC-RI). Concentrations may be varied to generate different specific weight average molecule weight fragment size populations independently of or in combination with other process parameters at any single weight average MW between 1 kDa and 250 kDa. This holds true for every parameter detailed here (please see Example 29 Process Parameters).

This solvent composition has been shown effective at dissolving and stabilizing sericin in solution such that it can be removed with the solvent. The cocoon/solvent ratio is 0.040 kg/kg-0.070 kg/kg (typically 0.042 kg/kg). An electric heater located at the base of the vessel is used to maintain temperature of the extraction solvent in the range of 30° C. to 110° C. For examples below, a measured temperature range between 94.5° C.-97° C., or 2.5° C. total temperature gradient was used. The elevated extraction solvent temperature, its maintenance, consistency and gradient within the reactor vessel drives sericin removal and final product creation with targetable specificity.

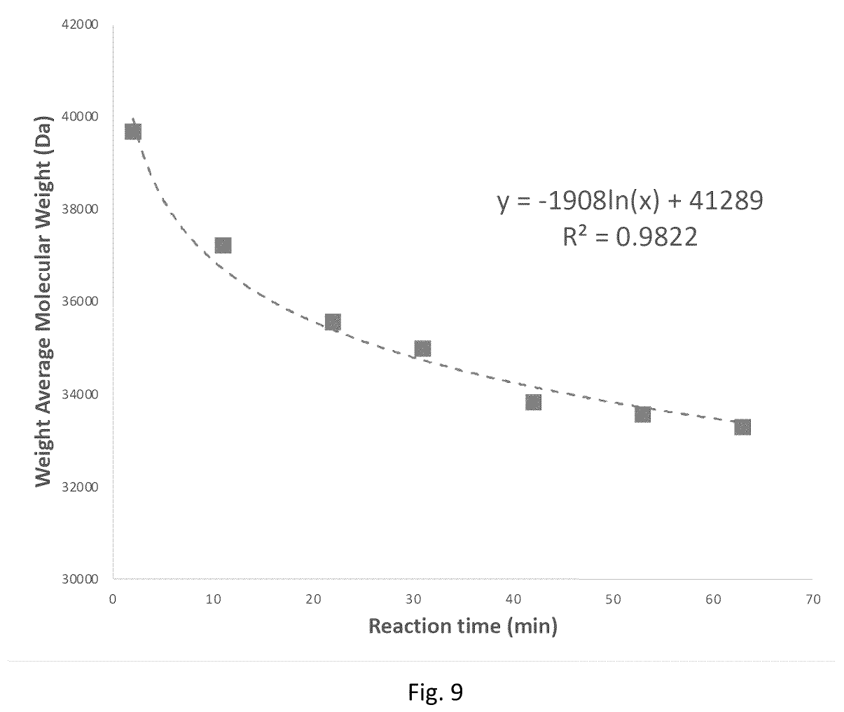

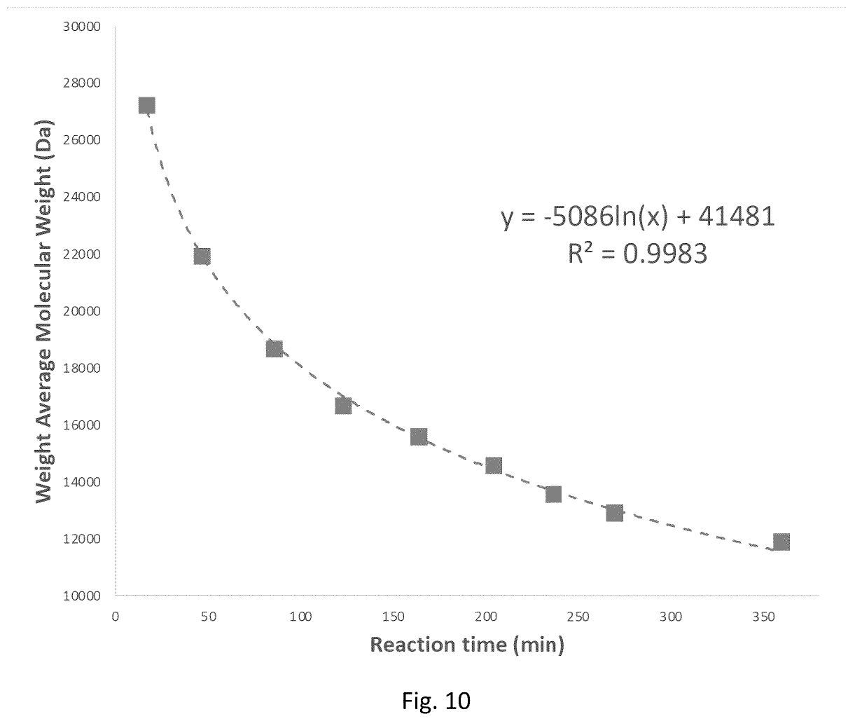

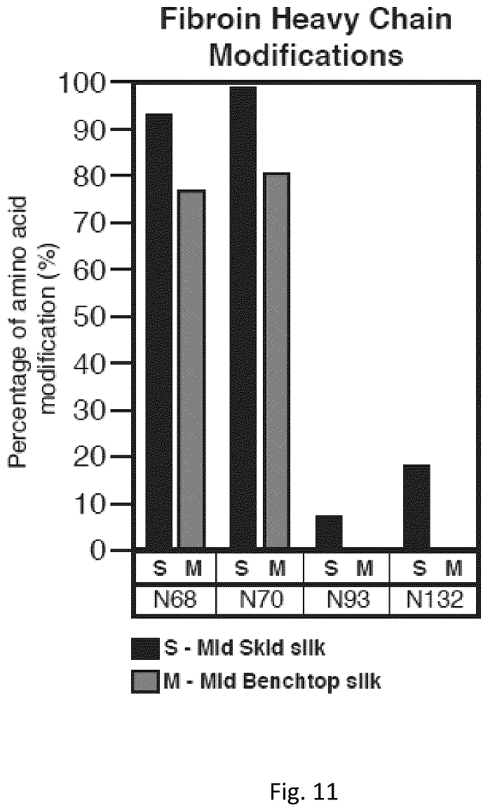

The rotating drum turns periodically throughout an isothermal phase of the extraction. It could rotate continuously, or intermittently, so long as the temperature gradient is maintained. In the examples below (FIGS. 9 and 10), a 30 minute isothermal phase was utilized. In another example, a 15 minute isothermal phase was utilized to generate a higher specific weight average molecule weight fragment size population of 96 kDa. This action serves to expose all fiber surfaces to the extraction solvent and to increase the precision of the product's final polydispersity, degree of modification, charge density, molecular weight and/or other critical features and benefits of the final product.

Rinsing with copious hot water follows, between 0.5× to 20× initial reaction vessel reaction solvent volume. The vessel is filled with non-potable water to partially submerge the perforated drum. Rinse water temperature is maintained in the range of 55° C.-65° C. with an ideal gradient of less than 10 degrees for 20 minutes with intermittent drum rotation, then the rinse water is drained to waste. This is repeated two additional times. Additional ranges are presented in Example 29 to detail expanded specific weight average molecule weight fragment size populations. The gradient or consistency or boundary of a specific temperature range is critical at every stage of the overall process or specific reaction step where increased or decreased temperature from ambient is employed, as these specific parameters and their associated levels or variable levels serve to produce differentiated final product from both processes and products described in the art.

Once rinsed, the drum rotates at high speed to remove water retained in the cocoons by centrifugal action. Fibroin with moisture content from 15%-65% wt (average 47%) is then manually removed from the washer and distributed evenly onto perforated trays. The residual moisture is driven off the fibroin by storage in a dryer with internal temperature maintained at 55° C.-60° C. until moisture content of the material is less than 1% of the total mass. It is also possible to continue to forward process the material immediately forward into the solvation process without drying. Extraction efficacy is verified by measuring the change in mass of the dry material before and after processing in the extraction unit. Typically, the amount of sericin removed from the cocoons is 30-36% wt of the total mass of the raw cocoons. The composition of the raw cocoons has been characterized by liquid chromatography-mass spec (LCMS). Using LCMS, sericin concentration in raw cocoons was determined to be ˜35.3% wt. Sericin was undetectable in fibroin after processing in the extraction unit. This result suggests that the sericin extraction method is effective, and the fraction of sericin detected in the raw cocoons corresponds well with the observed mass loss of the cocoons in the field.

### Fibroin Solvation and Modification

Control over the solubilization and modification of fibroin is achieved by dispersing the solid protein into a solvent and thermally treating the mixture at variable time and temperature. In one example, a 9.3 M lithium bromide solution in water is used as a solvent. The solvent is prepared in a vessel with or without baffles. The solution is blended to uniformity in the vessel using a center-mounted agitator with stacked 45° pitched blade turbines. Heat transfer oil circulates through the vessel jacket to stabilize bulk fluid temperature at the required reaction temperature while the solvent mixes. Typically, the reaction temperature is stabilized in the ranges of 100° C.-103° C. (103° C. target) or 122° C.-125° C. (125° C. target).

Fibroin is loaded into the vessel through an access port in the vessel head once the solvent reaches the required reaction temperature. The mass ratio of fibroin to solvent is typically 0.16 kg/kg. Downward force is applied to the floating protein mat to fully submerge the material and clear the headspace for additional material to be added. Once the headspace is cleared, agitation is briefly employed to disperse the wetted silk mat before addition is continued. Loading the vessel with the full mass of fibroin occurs over the course of 40-60 minutes, during which time reaction temperature is maintained in the vessel. The fibroin may also be placed in secondary containment and loaded at a single time point without the extended loading.

The reaction time begins after the fibroin addition is completed. Agitation is carried out over the full course of the reaction period. Reaction time varies depending on the desired properties of the resulting solution.

A first reaction process shows reactions carried out at multiple times between 0-63 minutes with working fluid temperature held at 100° C.-103° C. FIG. 1 displays the typical predictable evolution of the average molecular weight average (average MW) of the solubilized fibroin as a function of reaction time.

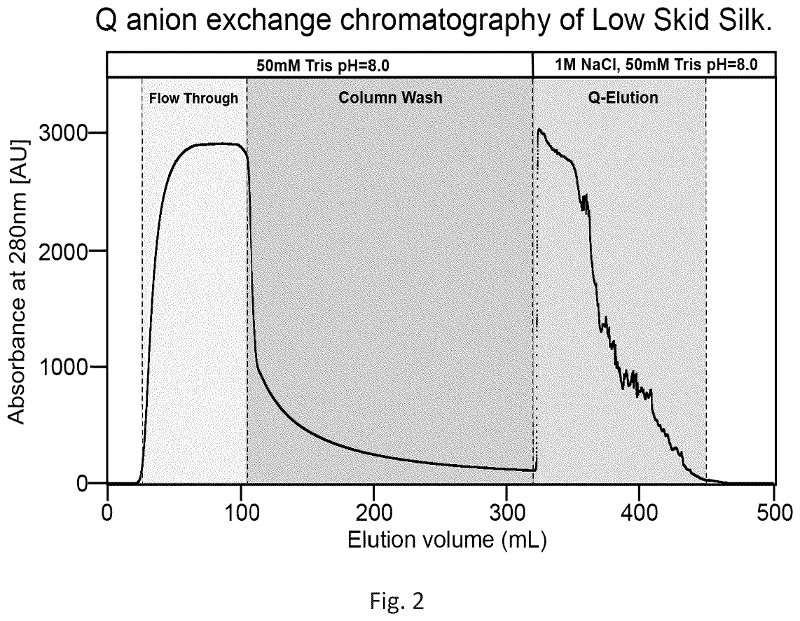

A second reaction process shows reactions carried out at multiple times between 40-420 min with temperature held at 122° C.-125° C. FIG. 2 displays the typical evolution of the average molecular weight average (average MW) of the solubilized fibroin as a function of reaction time.

The contents of the vessel are subsequently cooled. Cooling is accomplished by either of two methods. In one method, cooling is carried out by immediate removal of the solution from the vessel, dividing the solution into small volumes, and storing the containers in a refrigerator held at 4° C. In another method, cooling is carried out in place by recirculating chilled heat transfer oil through the vessel jacket. If cooling using a jacketed vessel, the temperature may be reduced to below 60° C. rapidly and within 70 minutes of the reaction period elapsing. Cooling to room temperature from 60° C. may be carried out rapidly or more slowly by environmental radiation or by forced cooling. When using forced cooling, the solution can be brought to room temperature within 3 hours, or less or more, to control the reaction outputs depending on desired product outcomes.

### Purification

The cooled reaction mixture is a viscous liquid composed of water, solvent or stabilizing salt (typically LiBr), fibroin, and miscellaneous undissolved organic solids. Fibroin is isolated from this mixture. Purification occurs through three filtration stages.

First, the reaction mixture undergoes dead-end filtration through a needle felt polypropylene filter media with nominal particle size rejection in the range of 1 mm-200 mm to remove relatively large undissolved contaminants. The filtered reaction mixture is transferred through the filtration media to a holding vessel with or without baffles, which is pre-charged with some volume of reverse osmosis/de-ionized (RODI) water. The volume of water charged to the holding vessel is determined by multiplication of the reaction mixture volume against a water-to-reaction mixture volumetric ratio. This ratio ranges from 1-7 L/L depending on the desired product and required downstream processing conditions.

The reaction mixture is blended to uniformity with the dilution water using a center-mounted agitator with stacked 450 pitched blade turbines or a propeller. Chilled propylene glycol circulates through the vessel jacket to cool the diluted mixture if the diluted material is to be stored for greater than 24 hours. Agitation for blending is limited to the bare minimum to achieve homogeneity, as excessive or prolonged shear on the fluid increases risk of product loss due to precipitation or foaming.

The diluted reaction mixture undergoes additional dead-end filtration through either a melt-blown and spun-bonded pleated poly propylene media with nominal 0.2 mm rejection or a resin bonded cellulose/diatomaceous earth lenticular media with absolute 2.5 mm rejection to reduce solution turbidity below a desired threshold.

The diluted reaction mixture is transferred through the filtration media to a tangential flow filtration (TFF) unit outfitted with a jacketed retentate vessel, a rotary lobe pump, 10 kDa molecular weight cutoff hollow fiber ultrafiltration membranes, or any molecular weight cut off between 1 kDa and 80 kDa, and an automatically controlled backpressure valve used to stabilize transmembrane pressure (TMP) during processing. TMP is defined as the average internal pressure of the TFF unit minus the permeate line pressure. The diluted reaction mixture recirculates between the retentate vessel and the membrane bank via the lobe pump and backpressure valve. The pump operates to maintain a constant recirculation flowrate, typically in the range of 200-500/min depending on application. The backpressure valve actuates to maintain TMP in the range of 7-35 psig depending on application. Chilled or heated propylene glycol or water is circulated through the retentate vessel to maintain working fluid temperature between 20° C. and 35° C. depending on application and with a filtration reaction temperate standard deviation of less than 10° C. ideally. For final product concentrations (protein by weight, in water) greater than 6% by weight, temperature greater than room temperature are typically used.

Operating under these conditions drives permeation of permeate such as water and LiBr through the membrane selective layers to waste. The TFF operation begins diafiltration, where volume is maintained in the retentate vessel by backfilling with RODI water during as volume is lost to membrane permeate. Diafiltration conditions are maintained until the conductivity of the permeate dips below a desired threshold, typically at any value between 10-50 mS/cm. LiBr concentration can be at any useful value, or typically below 1,000 ppm, or ideally be below 150 ppm. Diafiltration ceases once this condition is satisfied, at which point RODI water flow to the system stops and the working fluid is allowed to concentrate as permeation continues under maintained TMP and flow conditions. Protein concentration is monitored over the course of the concentration phase of operation. Concentration conditions are maintained until the protein concentration is within the range of 5-17% wt depending on application but can be any value between 1% to 40% by weight. Total residence time in the TFF unit ideally ranges from 12-35 hours depending on application.

The purified soluble silk fibroin fragment solution is drained from the TFF unit. The solution may be stored in either HDPE carboys or stainless-steel totes as work in progress or final product, or used in any further downstream process or processes, for example to further separate out ideal peptides, sub-populations of fragments or to purify the fragment population further, or complex the fragments with additional molecules or entities, or alter the form of the silk fibroin fragment solution from solubilized in water into dry powder by means of solvent extraction, lyophilization and freeze drying.

### Process Yield, Reproducibility, Consistency and Optimization

Process development has resulted in product yield improvements. Relative to the existing process described in Silk protein fragment compositions and articles manufactured therefrom (taken as the baseline case in this context), silk fibroin fragment yield was increased by at least 2× (200% yield) and up to at least 100×(10,000% yield improvement) for multiple fragment populations with specific average molecular weight averages between 1 and 250 kDa.

Additionally, process development has resulted in significant quality improvements exemplified by reduced variation in critical quality parameters such, specifically in production reproducibility between manufacturing production runs of weight average molecular weight and polydispersity characteristics of the protein fragment population in the final product. In reference to the standard bench top processes know in the art without the specific extraction reactor vessel and dissolution and filtration process parameters mentioned above, the scaleup of production of final product volumes of greater than 1 liter of soluble silk fragments in water resulted in up to 75% reduction in the standard deviation of molecular weight measurements and a 70% reduction in the standard deviation of dispersity measurements for a single silk fragment population with a single average MW between 1 and 250 kDa between 2 or more production runs.

### Example 4: Silk Use Categories

| | | Silk | | | |
|---|---|---|---|---|---|
| Use | | characterization | Source/ | Description | Data to |
| Category | Use Name | driver | Rationale | of use case | support |
| Personal | Barrier Redux | Breathable | Skin barrier | Facial serum | “Silk_pharmacology”. |
| Care | Emulsion | barrier formation, | benefits are | with | Clinical |
| | | claudin | a top | lightweight | data |
| | | upregulation | skincare | texture that | summarized in |
| | | | trend - | can either be | EBN-TDR-0073 |
| | | | Trans- | used alone to | with |
| | | | epidermal | support the | accompanying |
| | | | water loss is | skin barrier, or | report |
| | | | a | combined with | EVNCLI3M. |
| | | | measurement | other | Product spec: |
| | | | of skin | moisturizers/ | EBN-SPEC- |
| | | | barrier | serums. For | 0091 |
| | | | integrity. | use day or | |
| | | | Mid Skid silk | night. | |
| | | | at 2% | “Showcase | |
| | | | reduces | product” for | |
| | | | trans- | 33B. | |
| | | | epidermal | | |
| | | | water loss in | | |
| | | | clinical | | |
| | | | studies. | | |
| Personal | Skin Health | Breathable | Skin barrier | Touch-free | Formula is the |
| Care | Spray | barrier formation, | benefits are | eczema- | same as Barrier |
| | | claudin | a top | prone skin | Redux |
| | | upregulation, | skincare | treatment | Emulsion, but |
| | | TEWL reduction, | trend - | | with Low Skid |
| | | redness reduction | Trans- | | silk instead of |
| | | | epidermal | | Mid. Clinical |
| | | | water loss is | | study report |
| | | | a | | (PCR): |
| | | | measurement | | EVNCLI7M |
| | | | of skin | | |
| | | | barrier | | |
| | | | integrity. | | |
| | | | Mid Skid silk | | |
| | | | at 2% | | |
| | | | reduces | | |
| | | | trans- | | |
| | | | epidermal | | |
| | | | water loss in | | |
| | | | clinical | | |
| | | | studies. | | |
| Personal | Body Wash | Breathable | Skin barrier | Body wash for | Body wash |
| Care | | barrier formation, | benefits are | sensitive/eczema- | rinse-off study: |
| | | claudin | a top | prone skin | EBN-TDR- |
| | | upregulation, | skincare | | 0112. |
| | | remains on skin | trend - | | Consumer |
| | | after rinsing | Trans- | | study summary: |
| | | | epidermal | | EBN-TDR- |
| | | | water loss is | | 0108. |
| | | | a | | Consumer |
| | | | measurement | | study report: |
| | | | of skin | | (PCR): |
| | | | barrier | | EVNUSE1. |
| | | | integrity. | | Clinical study |
| | | | Mid Skid silk | | summary: EBN- |
| | | | at 2% | | TDR-0115. |
| | | | reduces | | Clinical study |
| | | | trans- | | report (PCR): |
| | | | epidermal | | EVNCLI4M |
| | | | water loss in | | |
| | | | clinical | | |
| | | | studies. | | |
| Personal | Mid Silk as a co- | Zeta potential | | Silk can be | Formula Stable |
| Care | emulsifier | and particle size | | used as an | at 10 weeks at |
| | | analysis in | | emulsifier in | high |
| | | progress | | combination | temperature |
| | | | | with a xanthan | concentrations. |
| | | | | gum and clay. | Additional study |
| | | | | Formulation | in progress to |
| | | | | can hold up to | further proof |
| | | | | 5% oil | that emulsions |
| | | | | | with different |
| | | | | | types of |
| | | | | | thickeners and |
| | | | | | clays can allow |
| | | | | | silk to be an |
| | | | | | emulsifier. |
| | | | | | Additional data |
| | | | | | supports that |
| | | | | | Silk and a |
| | | | | | rheology |
| | | | | | modifier works |
| | | | | | well |
| | | | | | without clay too |
| Personal | Collagen Redux | Silk upregulates | Adequate | Serum | HRIPT |
| Care | serum | collagen | collagen | formulated to | summary: EBN- |
| | | production in | production | visibly reduce | TDR-0101. |
| | | human dermal | minimizes | lines and | Phototoxicity/ |
| | | fibroblast cells | signs of | wrinkles, firm | sensitivity testing |
| | | | aging | skin and | summary: EBN- |
| | | | | improve | TDR-0138. |
| | | | | texture and | EpiOcular Test |
| | | | | tone for a | report |
| | | | | more youthful | summary: EBN- |
| | | | | appearance. | TDR-0134 |
| | | | | Features | |
| | | | | Activated | |
| | | | | Silk ™ 27P, a | |
| | | | | safe, naturally | |
| | | | | derived | |
| | | | | polypeptide | |
| | | | | solution that | |
| | | | | improves the | |
| | | | | appearance of | |
| | | | | fine lines | |
| | | | | without | |
| | | | | causing sun | |
| | | | | sensitivity or | |
| | | | | skin irritation. | |
| | | | | Ideal for | |
| | | | | consumers | |
| | | | | with sensitive | |
| | | | | skin looking | |
| | | | | for a safe/non- | |
| | | | | irritating | |
| | | | | product. | |
| Fabric | Laundry | Absorbency data | | Moisture | TXTL-715 |
| | treatment pods | | | management | Laundry pods |
| | | | | treatment | with Activated |
| | | | | system for | Silk; TDR-0140 |
| | | | | home use | POC |
| | | | | | dissolvable pod |
| | | | | | experiment |
| | | | | | summary |
| Fabric | Spray on | Absorbency data, | | Fabric | TXTL-715 |
| | treatment | hand feel | | treatment | Laundry pods |
| | | | | system for | with Activated |
| | | | | home use | Silk |
| Personal | Clear Mist with | zeta potential | Application | In the past Mid | |
| Care | Mid Silk | | work of | Silk is not able | |
| | | | cosmetic | be | |
| | | | formulation | | |
| | | | suggest that | | |
| | | | by | | |
| | | | suspending | | |
| | | | silk in a | | |
| | | | solution | | |
| | | | (very low | | |
| | | | viscosity.) | | |
| | | | Formulation | | |
| | | | can remain | | |
| | | | clear | | |
| Personal | Chemical | Stability matrix | Determining | AHA, BHA | Current data on |
| Care | Exfoliator on low | testing data with | most | and PHA | testing |
| | pH formulations. | different types of | common | | |
| | | acids, Zeta | chemical | | |
| | | potential | exfoliators | | |
| | | | used in | | |
| | | | cosmetics | | |
| | | | formulations | | |
| | | | that have | | |
| | | | compatibility | | |
| | | | with silk at | | |
| | | | low pH | | |
| Other | Food packaging | | | Edible/dissolvable | |
| | film | | | silk film | |
| | | | | use for | |
| | | | | packaging | |
| | | | | (dry/powder | |
| | | | | products) | |
| Personal | Deodorant/Antiperspirant | Visualization | An | Formation of | |
| Care | | (colorimeter) | antiperspirant | an “occlusive | |
| | | | salt can | plug” by the | |
| | | | change the | (+)′ly charged | |
| | | | charge on | silk which | |
| | | | the sweat | reverses a | |
| | | | glands | sweat gland's | |
| | | | | negative | |
| | | | | charge to a (+) | |
| | | | | charge on the | |
| | | | | skin surface, | |
| | | | | thus | |
| | | | | preventing | |
| | | | | sweat | |
| | | | | production | |

### Example 5. Modified Single Silk Fragment Weight Average Molecular Weight Average (MW) Populations “Low” and “Mid” with Degree of Amino Acid Modifications Produced Using New “Skid” Silk Production Processes

Modified Low and Mid Skid silk polypeptide compositions which are each a single silk fragment weight average Molecular Weight Average (MW) or average molecular weight average silk fragment population of silk-derived peptides, with each population having a polydispersity less than 5 and/or less than 3 as desired, described in this invention are never produced before compositions of silk-derived and modified polypeptides that when isolated display a wide range of behaviors, from extreme self-assembly to superb solubility and stability over time in various buffers and various weight average molecular weights and polydispersities. These novel silk-derived polypeptide compositions contain unique modified amino acids that result from our unique silk “skid” processing method and scale. The tight controls around temperature, silk concentration, salt concentrations, physical agitation and purification allow us to tune at each step of the process the unique peptide compositions in our silk species to design for specific performance criteria. Some of constituent polypeptide compositions display biological activity and could be used as therapeutic candidates.

Silk is a versatile material that can be used in many applications from development of implantable medical devices to the development of soluble polypeptide formulations of medicinal value. A major challenge with silk polypeptides in solution is their tendency to self-assemble and aggregate, making the control of their solubility very difficult. Also, the kinetics of gel/film formation cannot be controlled in a predictable way. Our novel silk peptide compositions contain populations of peptides that allow us to control their properties and develop products with predictable and desired properties.

### Development of Low and Mid Skid Silk/Modified Polypeptide Compostions.

Activated silk contains a collection of many polypeptides with different properties. Silk fragment populations has been characterized mostly based on its average molecular weight average and polydispersity, referred to here as a new silk fragment population or “silk polypeptide”. So far no mixture of silk polypeptides (i.e., multiple silk fragment populations) have been characterized or has been generated. Here, a unique large-scale process known as the Skid Silk Process or “skid: silk” was used to generate compositions of silk polypeptides.

Low/Mid skid silks were produced using process parameters and conditions described. Briefly, in summary, they begin their process to remove sericin using sodium carbonate at specific silk mass, sodium carbonate, and water ratios. Multiple different temperature washing cycles between 100° C. and 60° C. and agitation is also key in producing the specific natural/modified compositions. The silk is then dried to remove water at a specific temperature that maintains the silk composition. Next the silk is dissolved in a high concentration of Lithium Bromide at 103° C. and 125° C. for 1 and 6 hours respectively. The time and temperature allow for fine tuning the degree of post translational modifications that give the unique polypeptide compositions. The silk is then purified to remove Lithium Bromide and concentrate the silk.

### Generation of Low and Mid Skid Silk/Modified Polypeptide Compositions.

Silk was washed to remove sericin at 100° C. and 60° C. with sodium carbonate and then dried at 60° C. The silk was then dissolved in 9.3 M Lithium Bromide at 103° C. for 1 hour for Mid silk and 9.3 M Lithium Bromide at 125° C. for 6 hours for Low silk. This dissolution step controls not only molecular weight but also the polypeptide modifications creating the natural/modified silk compositions. The silk was then filtered to remove undissolved debris and purified using 10 kDa cutoff PES hollow fiber membranes and concentrated using the same process leaving only natural/modified silk composite in solution with pure water. Every unit ops was tightly controlled for temperature, time, concentrations, agitation, and shear.

The Silk Preparations have Unique Modifications Depending on the Production Method.

The dissolution of degummed silk cocoons was performed in high concentration of chaotropic salts (9M LiBr) and at very high temperatures that exceed 100° C. (see previous sections). The unique thermal treatment that occurs during the production method described herein, promotes the deamidation of Asparagine and Glutamine residues and the oxidation of Methionines. The deamidation of Asparagine and Glutamine residues and the oxidation of Methionines is referred to as “modifications” from now on. To determine the degree of amino acid modification during the various silk preparation methods LC/MS approaches were used (see LC/MS analysis of polypeptides for more details). The Skid process is run in facilities known as Walpole and Medford. A former process not employing the new process conditions is referred to as “benchtop” silk, or the “bench silk process” known in the art.

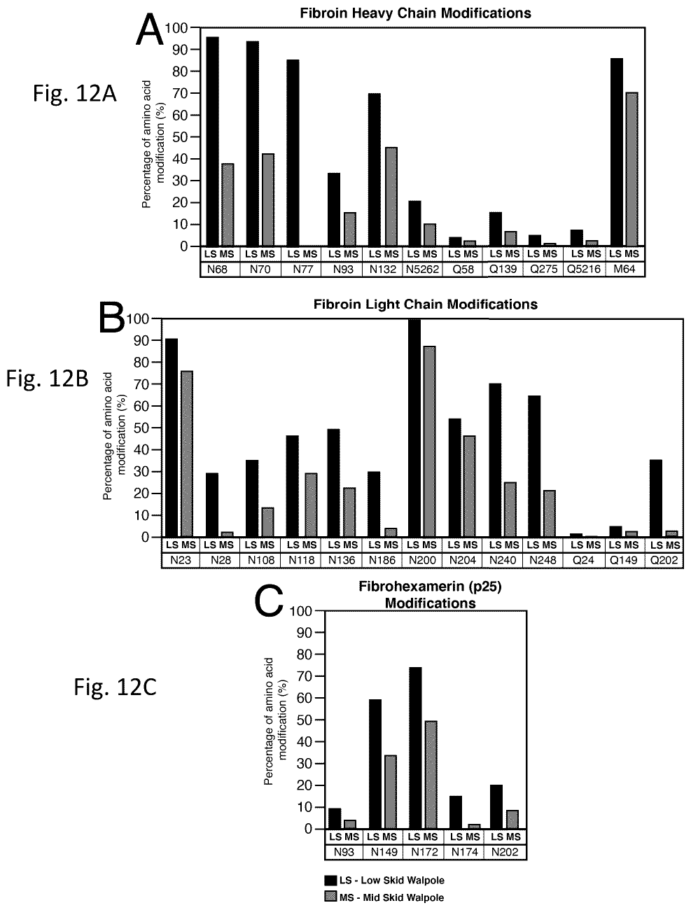

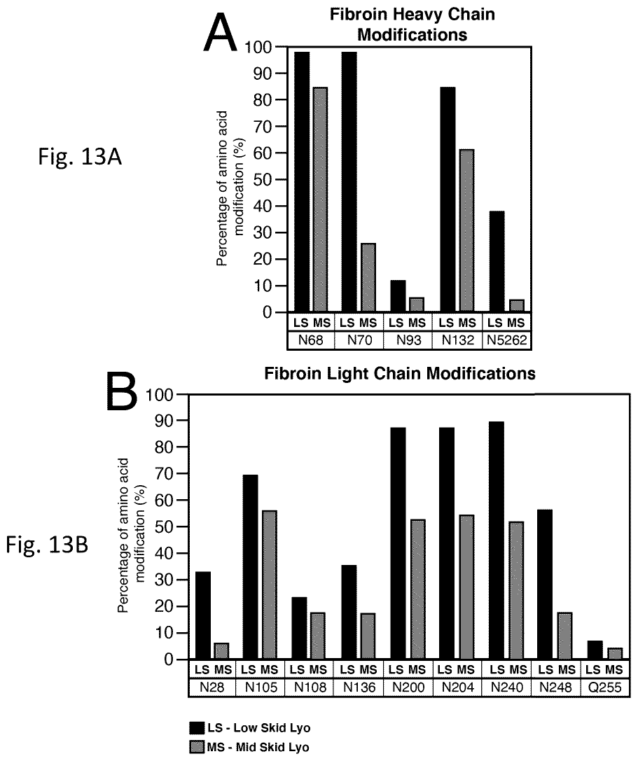

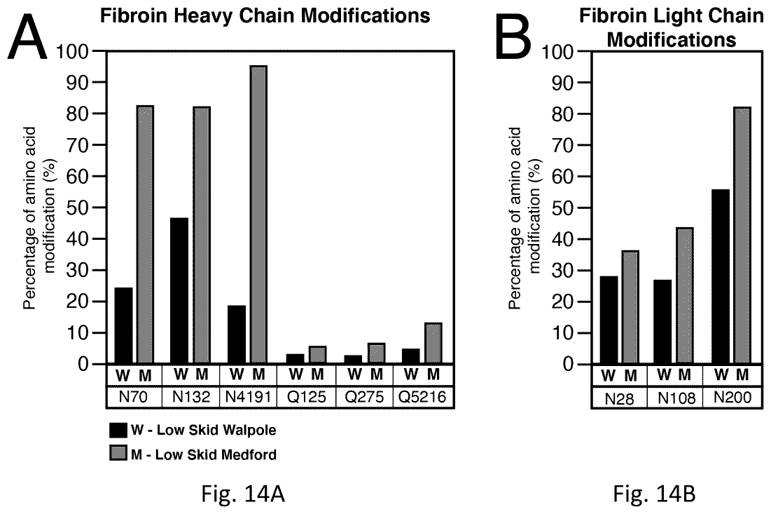

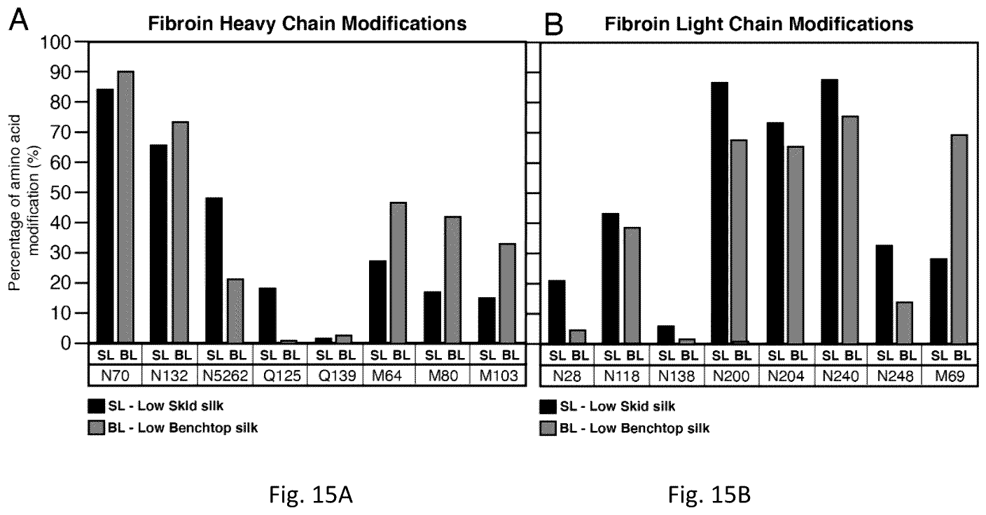

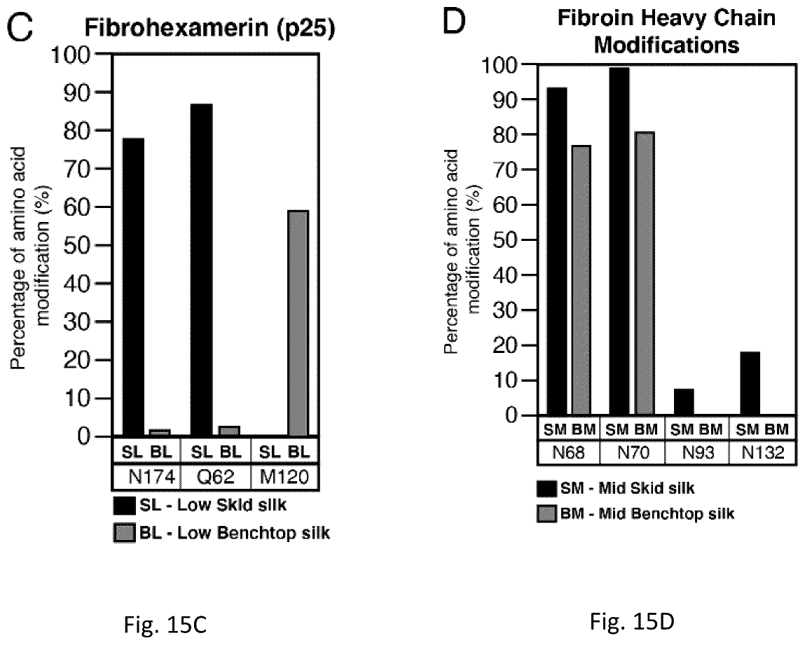

When Low Skid Silk was compared with Mid Skid silk produced with differing process conditions in the Walpole facility, it was found that Low Skid silk was more modified than Mid Skid silk (FIGS. 12A-12C, Table 2). When silk produced in Walpole was lyophilized it retained the same modification trend; lyophilized Low Skid silk was more modified than lyophilize Mid Skid silk (FIGS. 13A-13B, Table 3). Low Skid silk produced in the Walpole facility is unique and less modified than the Low Skid silk produced in Medford (FIGS. 14A-14B, Table 4). When silk was produced using a benchtop setup, the resulting silk preparation was different than its Skid counterpart. On average Low Benchtop is less deamidated than Low Skid silk. (FIGS. 15A-15C, Table 5) Mid Benchtop on average is also less modified than Mid Skid silk (FIG. 15D, Table 5 comparing Mid Skid silk with Mid Benchtop silk).

### LC/MS Analysis of Polypeptides.

### Materials

### Reagents

Guanidine hydrochloride (GuHCl) (Sigma cat #G3272-1KG)
Dithiothreitol (DTT) (ThermoFisher cat #20290)
Iodoacetamide (IAM) (Sigma cat #I1149-5G)
HPLC-grade water (FisherChemical cat #W5-4)
Acetonitrile (ACN) (FisherChemical cat #A955-4)
Formic acid (FA) (FisherChemical cat #A117-10X1AMP)
Trifluoroacetic acid (TFA) (FisherChemical cat #A116-10X1AMP)
Sodium acetate (Sigma cat #S5636-250G)

### Proteases

Trypsin/Lys-C mix (Promega cat #V5073)
Chymotrypsin (Promega cat #V1061)
Glu-C (Promega cat #V1651)

### Solutions

6 M GuHCl
50 mM DTT (10×)
100 mM IAM (10×)
50 mM sodium acetate

### Methods

### Denaturation, Reduction and Alkylation

Samples were stored at 4° C. until used for analysis.

For each sample, an aliquot was taken and mixed with an equal volume of 6 M guanidine hydrochloride (GuHCl) in a new tube. 50 mM dithiothreitol (DTT) was added to a final concentration of 5 mM and the samples were incubated at 60° C. for 30 minutes. After a brief equilibration period to room temperature, 100 mM iodoacetamide (IAM) was added to a final concentration of 10 mM and the samples were incubated at room temperature in the dark for 30 minutes. The IAM reaction was quenched by the addition of 50 mM DTT to a final concentration of 5 mM DTT. The samples were diluted in 50 mM sodium acetate to get a final concentration of 0.18 M GuHCl.

### Protease Digestion

Using the sample concentrations provided, 3 aliquots corresponding to 30 μg of total protein were taken in separate tubes. Samples were then treated with enzymes at a protease to protein ratio of 1:30 (1 μg of each protease) overnight at either room temperature (chymotrypsin) or 37° C. (trypsin/Lys-C and Glu-C). The aliquots treated with trypsin/Lys-C and Glu-C were boosted with the same amount of enzyme and incubated at 37° C. for 3 hours the next day. The protease reactions were quenched by the addition of TFA to a final concentration of 1% (v/v). Samples were centrifuged for 10 minutes at 14,000 rpm and supernatant was transferred to HPLC autosampler vials for LC-MS analysis.

### LC Conditions

Column: C18 column (100 μm×200 mm, 3 μm)
Mobile Phase A: Water 0.1% formic acid
Mobile Phase B: Acetonitrile 0.1% formic acid
Flow Rate: 300 nL/min (micro pump) and 15 μL/min (loading pump)
Chromatographic time: 60 minutes
Elution Gradient: 14% B (0 to 3.3 min); 14% to 30% B (3.3 to 35 min); 30% to 95% B (35-37 min); 95% to 80% B (37-39 min); 80% B (39-41 min); 80% to 14% B (41 to 44 min); 14% B (44 to 60 min)
Injection amount: 2 ug

### MS Conditions

Acquisition for full MS ranges from 350 to 1600 m/z. The MS method is based on data-dependent acquisition(DDA) for the top 10 ions with an isolation window of 3.0 m/z and a normalized collision energy of 27.

### Data Acquisition and Analysis

Data was acquired using Thermo Xcalibur™ Software. Data analysis was performed using Thermo Proteome Discoverer™ Software. In order to unequivocally assign a specific protein from the identified peptides, a minimum of 2 unique peptides per protein are required upon searching against Bombyx mori database.

For each modification site, all the peptides containing that amino acid were categorized into modified vs unmodified. The modification percentage is then calculated using the formula below:

$$modification \% = \frac{sum of modified peptides}{sum of modified and unmodified peptides}$$

### Tables

TABLE 2
Percentage of amino acid modifications in Low Skid Silk and Mid Skid silk produced
in the facility in Walpole. N are Asparagines that become aspartic acid and Q are
Glutamines that become deamidated. M corresponds to Methionies that become oxidized.
Fibroin Heavy Chain

| | N68 | N70 | N77 | N93 | N132 | N5262 | Q58 | Q139 | Q275 | Q5216 | M64 |
|---|---|---|---|---|---|---|---|---|---|---|---|
| LS Walpole | 95.37 | 92.99 | 85.33 | 33.73 | 69.08 | 21 | 3.56 | 15.97 | 4.01 | 6.98 | 86.54 |
| MS Walpole | 37.25 | 42.18 | 0 | 15.81 | 45.43 | 10.14 | 2.95 | 6.9 | 0.91 | 2.49 | 70.7 |

Fibroin Light Chain

| | N23 | N28 | N108 | N118 | N136 | N186 | N200 | N204 | N240 | N248 | Q24 | Q149 | Q202 |
|---|---|---|---|---|---|---|---|---|---|---|---|---|---|
| LS Walpole | 90.69 | 28.26 | 34.4 | 45.7 | 48.88 | 29.79 | 100 | 53.44 | 70.34 | 63.91 | 0.74 | 3.58 | 35.16 |
| MS Walpole | 75.49 | 1.58 | 12.92 | 28.46 | 22.06 | 3.98 | 87.36 | 45.88 | 25.12 | 20.86 | 0.04 | 2.65 | 2.58 |

Fibrohexamerin (p25)

| | N93 | N149 | N172 | N174 | N202 |
|---|---|---|---|---|---|
| LS Walpole | 9.76 | 59.34 | 74.42 | 15.42 | 20.19 |
| MS Walpole | 4.38 | 34.42 | 50.03 | 2.58 | 9.45 |

TABLE 3
Percentage of amino acid modifications in Low Skid Silk and Mid Skid
silk produced in the facility in Walpole and lyophilized. N are
Asparagines that become aspartic acid and Q are Glutamines that
become deamidated. M corresponds to Methionies that become oxidized.
Fibroin Heavy Chain

| | N68 | N70 | N93 | N132 | N5262 |
|---|---|---|---|---|---|
| Low Skid Lyo | 100 | 100 | 11.87 | 86.34 | 39.13 |
| Mid Skid Lyo | 86.1 | 26.57 | 5.95 | 63.12 | 4.37 |

Fibroin Light Chain

| | N28 | N105 | N108 | N136 | N200 | N204 | N240 | N248 | Q255 |
|---|---|---|---|---|---|---|---|---|---|
| Low Skid Lyo | 32.21 | 69.79 | 22.84 | 34.57 | 87.31 | 87.1 | 90.45 | 56.16 | 7.02 |
| Mid Skid Lyo | 4.06 | 56.15 | 17.14 | 15.53 | 52.87 | 54.44 | 52.37 | 17.79 | 2.4 |

TABLE 4
Percentage of amino acid modifications in Low Skid silk produced
in the facility in Walpole and Medford under differing process
parameters and variable levels. N are Asparagines that become
aspartic acid and Q are Glutamines that become deamidated.
M corresponds to Methionies that become oxidized.
Fibroin Heavy Chain

| | N70 | N132 | N4191 | Q125 | Q275 | Q5216 |
|---|---|---|---|---|---|---|
| Low Skid Walp | 25.02 | 47.21 | 18.78 | 2.82 | 2.92 | 4.39 |
| Low Skid Med | 83.82 | 82.88 | 96.01 | 5.82 | 6.7 | 13.67 |

Fibroin Light Chain

| | N28 | N108 | N200 |
|---|---|---|---|
| Low Skid Walp | 27.94 | 26.16 | 54.96 |
| Low Skid Med | 35.38 | 42.92 | 80.99 |

TABLE 5
Percentage of amino acid modifications in Mid silk produced in
between the different Skid and Benchtop processes. N are Asparagines
that become aspartic acid and Q are Glutamines that become deamidated.
M corresponds to Methionies that become oxidized.
Fibroin Heavy Chain (Low Skid and Low Benchtop)

| | N70 | N132 | N5262 | Q125 | Q139 | M64 | M80 | M103 |
|---|---|---|---|---|---|---|---|---|
| Low Skid Walpole | 85.12 | 66.91 | 48.31 | 2.66 | 0.62 | 27.9 | 17.29 | 16.1 |
| Low Benchtop | 90.56 | 74.64 | 21.29 | 1.2 | 2.59 | 47.54 | 43.14 | 33.3 |

Fibroin Light Chain (Low Skid and Low Benchtop)

| | N28 | N118 | N138 | N200 | N204 | N240 | N248 | M69 |
|---|---|---|---|---|---|---|---|---|
| Low Skid Walpole | 21.09 | 43.51 | 5.37 | 87.48 | 74.02 | 88.49 | 31.93 | 28.61 |
| Low Benchtop | 4.72 | 38.96 | 0.83 | 68.27 | 66.14 | 75.25 | 12.87 | 69.6 |

Fibrohexamerin (p25) (Low Skid and Low Benchtop)

| | N174 | Q62 | M120 |
|---|---|---|---|
| Low Skid Walpole | 77.6 | 87.34 | 0 |
| Low Benchtop | 0 | 2.74 | 59.28 |

Fibroin Heavy Chain (Mid Skid and Mid Benchtop)

| | N68 | N4191 | Q113 | Q125 |
|---|---|---|---|---|
| Mid Skid Walpole | 94.29 | 100 | 7.07 | 17.95 |
| Mid Benchtop | 77.81 | 81.3 | 0 | 0.57 |

*No significant differences in the other proteins between Mid Skid and Mid Benchtop.

### Calculation of the Percentage of Amino Acid Modification Per Location Along the Amino Acid Sequence of Each Protein Chain.

To determine the percentage of modified amino acids within the sequences of individual protein chains in the silk preparations, LC/MS analysis was employed. Through this analytical method, it was not only discerned the sequence of all peptides present in the solution but also quantified their respective concentrations. LC/MS methodology possesses the capability to pinpoint amino acid modifications at specific positions within these sequences, a phenomenon induced by the production techniques. Upon successfully identifying and quantifying all the peptides within the mixture, they were aligned with the protein sequences present in the silk cocoons. This alignment process allowed for the ascertainment of the origin of each peptide along the polypeptide chain of fibroin heavy and light chains, as well as fibrohexamerin (p25). Consequently, the exact positions of amino acids were pinpointed on these polypeptide chains.

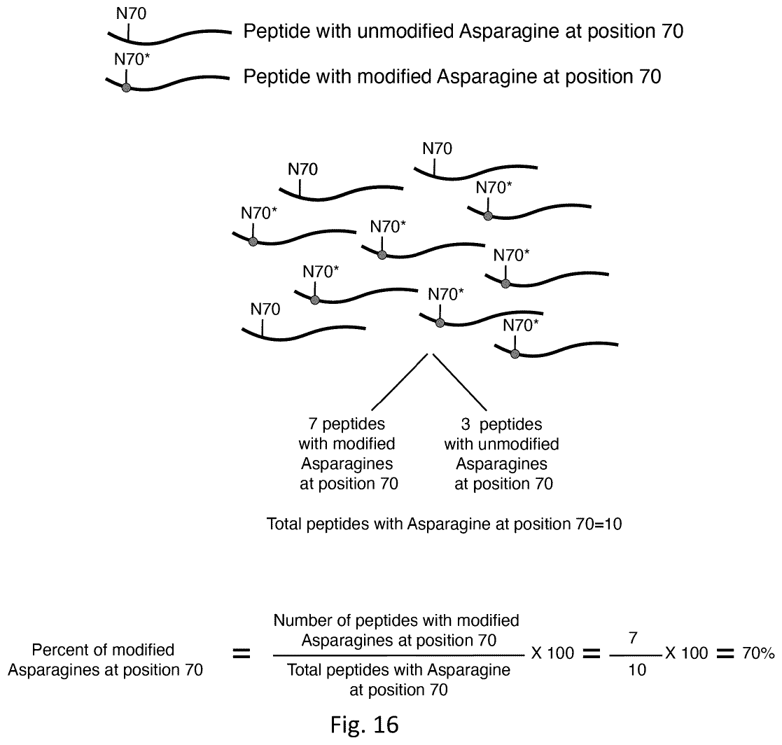

To calculate the percentage of modified amino acids at specific locations, all peptides containing these amino acids were quantified, both modified and unmodified. The calculation involved dividing the quantity of peptides containing modified amino acids by the total quantity of peptides containing those particular amino acids (modified and unmodified) and then multiplying the result by 100. This yielded the percentage of modified amino acids at the designated location. Refer to FIG. 16.

### Example 6. Low Skid Silk/Modified Polypeptide Compositions Isolated by Charge and Size Properties

Described Herein is a Novel Method to Generate Compositions of Polypeptide that are derived from B. mori silkworm cocoons and comprise of natural and modified polypeptides. This novel composition is called Low Skid silk/modified polypeptide compositions.

The novel production method involves removing sericin through several washing steps with an organic sodium carbonate salt with tightly controlled multi-stage temperature cycles and agitation as the first step in forming natural/modified polypeptide composition. Next the silk is dried to remove remaining water at controlled temperature to maintain polypeptide composition. The silk is then dissolved in high concentration of Lithium salt at 125° C. for 6 hours to achieve the compositions of Low Skid silk. The liquid solution is then filtered and purified to remove the Lithium salt leaving only the natural/modified silk compositions in solution with pure water.

Low Skid silk/modified polypeptide compositions comprise of populations of silk/modified polypeptides with distinctive properties.

Low Skid silk/modified polypeptide composition does not self-assemble at 5 mg/mL. Low Skid silk/modified polypeptide composition comprises of a variety of populations of silk/modified polypeptides; distinct populations were isolated based on charge and size, by fractionating Low Skid silk/modified polypeptides by anion exchange chromatography and size exclusion chromatography. A high-resolution separation of five negatively-charged silk compositions was achieved—AS77, AS78, AS79, AS80, and AS81. These silk compositions differ from one another by their average size, when AS77 is the largest, and AS81 is the smallest. These silk compositions do not self-assemble under conditions that promote self-assembly at 5 mg/mL.

The Low Skid silk/modified polypeptide compositions described in this invention are novel compositions of silk and modified polypeptides composed of a variety of silk polypeptide populations, generated by the exclusive treatment method of natural silk produced by B. mori. These silk compositions contain modified amino acid sequences that result from silk processing method and scale. The tight controls over temperature, silk concentration, buffers and salt concentrations, physical agitation, and purification allow for the precise development of silk compositions with a variety of performance criteria. Isolation of these populations by charge and size reveals new characteristics, like high solubility and stability in solution over time in these populations. The purification method allows for the isolation of silk/modified polypeptide compositions that display biological activities and could be used for therapeutic purposes.

Silk is a complex natural biomaterial that has the potential to be utilized in various applications such as the development of implantable medical devices, and the development of soluble polypeptide compositions of medical value. Additionally, it was demonstrated that silk peptides have anti-genotoxic effects. However, silk, in its natural form, is not soluble, and silk polypeptide compositions, without the proper processing, display poor solubility in solution and tend to self-assemble and aggregate over time. The kinetics of this self-assembly is unpredictable, and highly depends on the composition of the silk polypeptides/modified composition. Novel silk/modified polypeptide compositions were produced and the silk/modified polypeptide compositions isolated specific populations within these compositions. The isolation process allows for the control of the properties of the silk compositions and development of products with predictable and desired characteristics.

### Generation of Low Skid Silk/Modified Polypeptide Compositions.

Silk is washed to remove sericin at 100 C and 60 C with sodium carbonate and then dried at 60 C. The silk is then dissolved in 9.3M Lithium Bromide at 125 C for 6 hours. This dissolution step controls not only molecular weight but also the polypeptide modifications creating the natural/modified silk compositions. The silk is then filtered to remove undissolved debris and purified using 10 kDa cutoff PES hollow fiber membranes and concentrated using the same process leaving only natural/modified silk composite in solution with pure water. Every unit ops is tightly controlled for temperature, time, concentrations, agitation, and shear.

### Isolation of Low Skid/Modified Polypeptide Compositions

### Isolation of the AS77-AS81 Silk/Modified Polypeptide Composition Component of Low Skid Silk/Modified Polypeptide Composition.

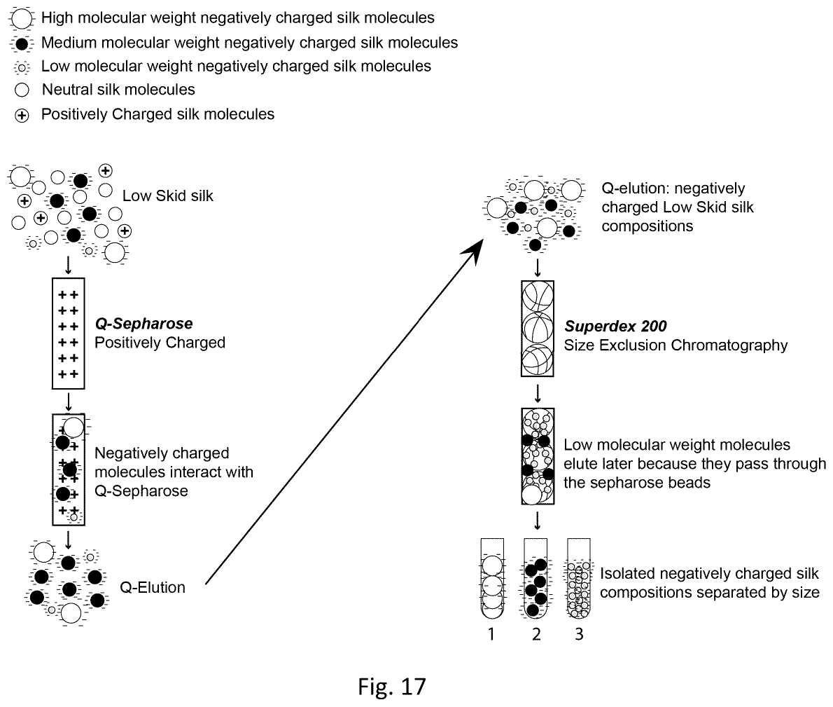

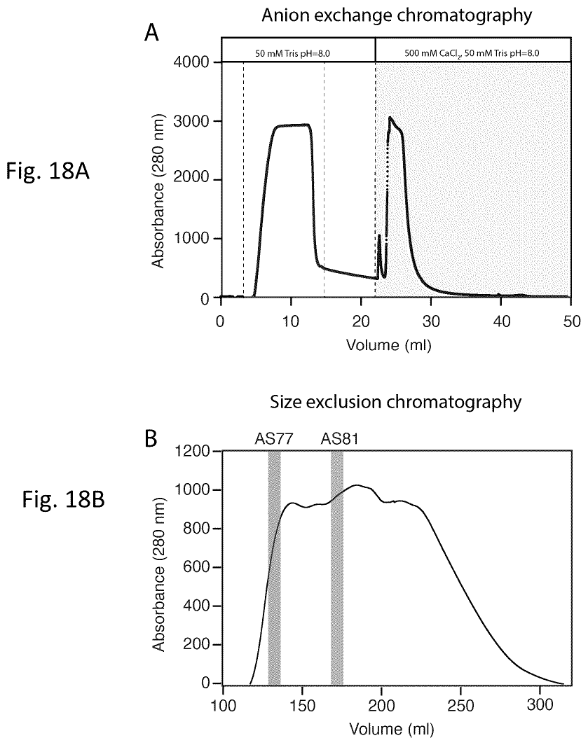

To isolate AS77-AS81 Low Skid silk/modified polypeptide compositions were fractioned using anion exchange chromatography (Q-Sepharose chromatography), following HiLoad 26/600 Superdex 200 μg size exclusion chromatography of the Q-eluate (FIG. 1, 2). Prior to chromatography, Tris was added to the silk preparations to a final concentration of 50 mM Tris-HCl, pH=8.0. The silk was centrifuged and filtered before loading to the Q-Sepharose column, to remove any preformed aggregates. The silk compositions were loaded onto the Q-Sepharose column, and the flowthrough fraction was collected. The negatively charged silk compositions were eluted using high salt buffer (50 mM Tris, 500 mM CaCl₂)). The eluted fractions were pulled together and are referred to as the Q-elution fraction. The Q-elution was further fractionated by the HiLoad 26/600 Superdex 200 μg, where the largest polypeptide compositions were eluted first, and each following fraction had a population of lower molecular weight of silk compositions (FIGS. 17, 18A, 18B). Low Skid silk preparation solutions have a characteristic yellow hue. The Q-elution fraction has a strong yellow hue, while the flowthrough fraction is transparent, and tends to self-assemble very quickly. The Q-elution silk compositions that are fractionated by size exclusion also had a yellow hue.

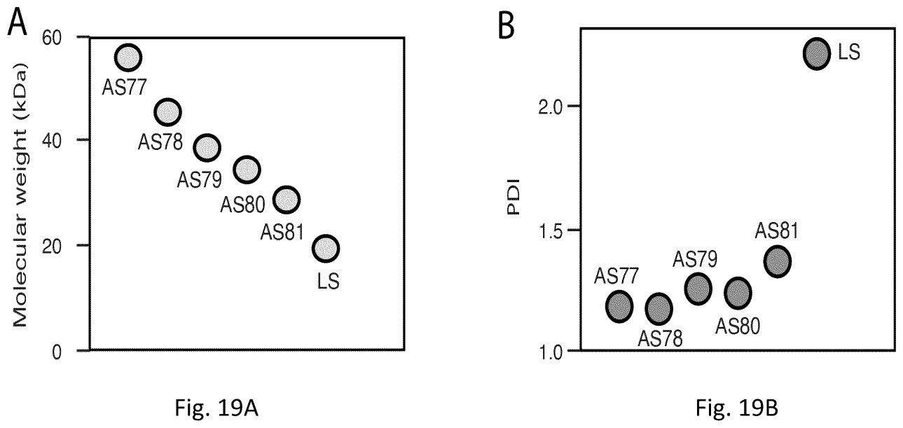

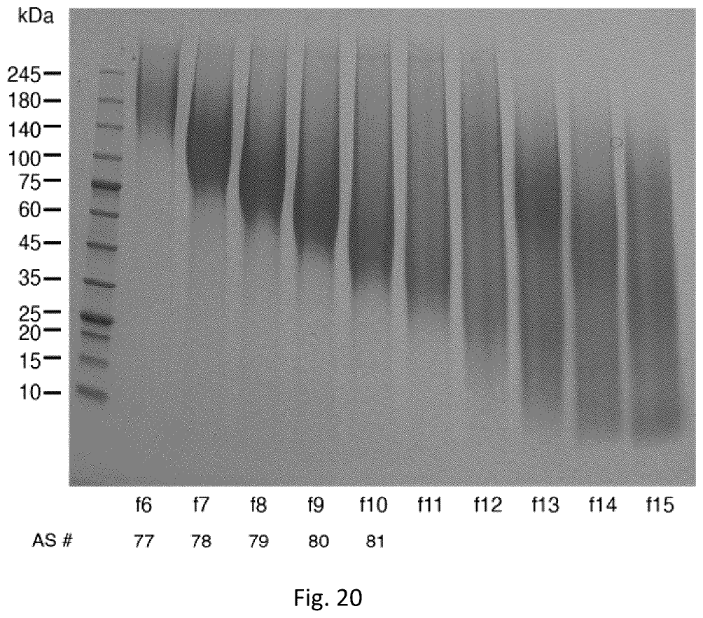

When silk formulations AS77-AS81 are analyzed with an analytical SEC column (see materials and methods) with HPLC, each of the silk formulations demonstrates a different average Mw, and a different Polydispersity (PDI) value (FIGS. 19A-19B, Table 7). In general, AS77 has the highest Mw (55714 Da), while AS81 has the lowest Mw (28750 Da). The PDI values display a differential change as well. The PDI value of AS77 is relatively low (1.1836), and AS81 is higher (1.3648) (FIGS. 19A-19B, Table 7). Unfractionated Low Skid silk has an average Mw of ˜19500, indicating that most of the peptide population tends to have lower molecular weight than fractions AS77-AS81. The polydispersity of unfractionated Low Skid silk is ˜2.2—significantly higher than the values of fractions AS77-AS81. This indicated that the unfractionated Low Skid silk is composed of a much diverse peptide population compared to fractions AS77-AS81.

### AS77-AS81 Silk Compositions Demonstrate Relative Uniformity by Dynamic Light Scattering and Show Gradual Particle Size Distribution.

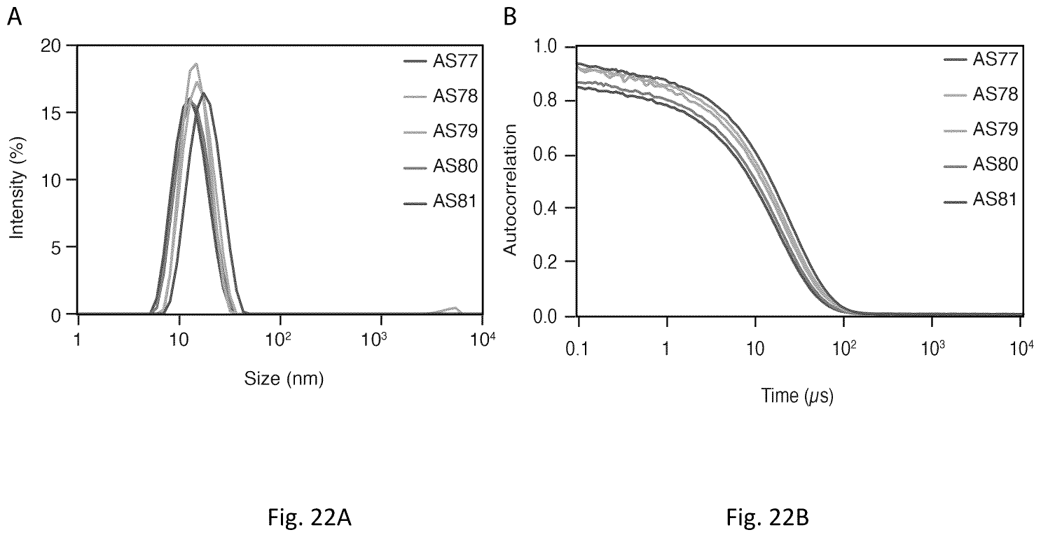

In dynamic light scattering analysis (Zetasizer Pro, FIGS. 22A-22B, Table 6), AS77-AS81 demonstrated relatively uniform, though broad, peaks where AS77 has the largest Z-average (17.16 nm), then AS78 (15.14 nm), and so on (Table 6), demonstrating the efficiency of the fractionation by size of the Q-elution fraction.

### Self-Assembly of Low and Mid Skid/Modified Polypeptide Compositions

Self-Assembly Assay and Data Derived from it

To study the stability of silk/modified peptide compositions in solution, Self-Assembly assays at a concentration of 5 mg/mL were performed.

The absorbance at 550 nm curves of the self-assembly assays are sigmoid and they can be described as logistic curves. The typical logistic function is:

$$f ( x ) = \frac{{A}_{max}}{1 + {e}^{- k ( t - t 0.5 )}}$$

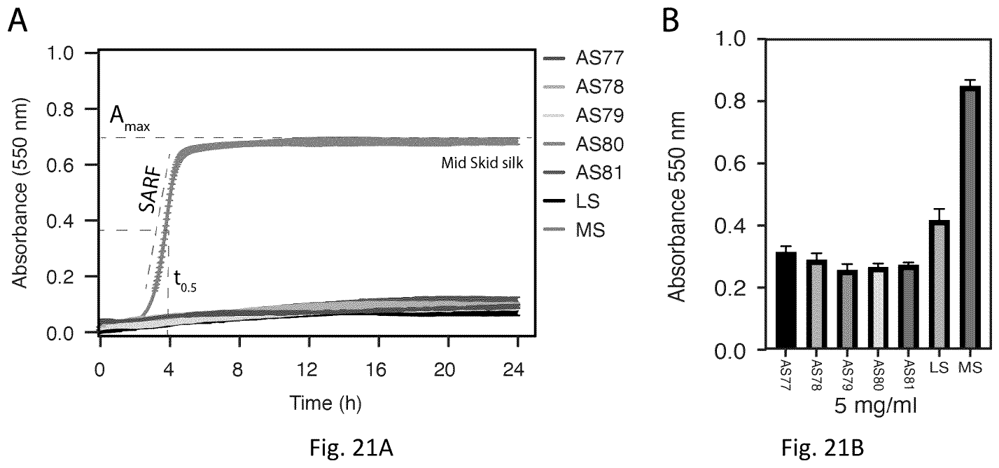

Aₘₐₓ is the maximum density of the gel formed
k is the Self-Assembly Rate Factor (SARF)
t_{0.5} is the time point at which 50% of the gel has formed
e is the exponential equation for the specific curve
(see FIG. 21A the red dotted lines for a better demonstration of how these factors from the Self-Assembly experiments were calculated)

Another parameter introduced to characterize the propensity of silk to form gels is the Self-Assembly Factor (F_{SAF}) which is:

$${F}_{SAF} = \frac{1}{t 0.5 ( min )} * {A}_{max} ( Abs ) * 1000$$

Using the experimental data from the Self-Assembly assays that were performed with the various novel isolated silk polypeptides, these parameters were calculated and used to dissect their properties (FIGS. 21A-21B). Four parameters were focused on, collectively referred to as Self-Assembly kinetic factors; the Self-Assembly Rate Factor (SARF), Aₘₐₓ, t0.5, and the Self-Assembly Factor (SAF) (FIGS. 21A-21B). The SARF shows how fast silk self-assembles to form gel after the reaction begins or the gelation nuclei have formed; Aₘₐₓ shows how dense is the gel that is formed after self-assembly is complete, t0.5 shows how long it takes for the self-assembly reaction to reach the point where gel density is

$$\frac{{A}_{max}}{2}$$

and SAF shows the propensity of silk to self-assemble (FIGS. 21A-21B).

### AS77-AS81 Silk Compositions do not Self-Assemble.

Self-assembly assays revealed that Low Skid silk/modified peptide compositions do not self-assemble under the experimental system conditions (FIG. 22A). No self-assembly occurred after 24 hours, and no self-assembly was detected even 12 days post-assay (FIG. 22B). Mid Skid/modified peptide compositions was used as a positive control and shows fast self-assembly kinetics (FIGS. 21A-21B).

Materials and Methods used for the generation and characterization of AS77-AS81.

### Anion Exchange Chromatography of Silk.

Low Skid silk was provided at a concentration of 60 mg/mL. 50 mM Tris, pH=8.0 buffer was added to the Low Skid silk, and the silk was centrifuged at 16000 rpm (rotor JA-18, Beckman coulter, average of 28100×g), at 4° C., for 30 min to separate formed aggregates from soluble silk. The supernatant was collected and filtered through a 0.22 μm PES filter. For silk fractionation, Q-Sepharose prepacked columns connected to an AKTA pure 25 L or HiPrep Q FF 16/10 20 mL Column, or HiTrap™ Capto™ Q 1 mL column was used. All buffers used were filtered through a 0.22 μm PES filter and degassed with sonication. Centrifuged and filtered Low Skid silk was loaded on 5×5 mL HiTrap Q HP columns washed with 10 column volumes of 50 mM Tris pH=8.0, 10 column volumes of 50 mM Tris pH=8.0, 500 mM CaCl₂) and finally 10 column volumes of 50 mM Tris pH=8.0. 170 mL of centrifuged Low Skid silk were loaded on the column with a flow rate of 5 mL/min. The flow-through was collected. The column was washed with 50 mM Tris pH=8.0 until the absorbance at 280 nm [A₂₈₀] got to 100 AU. Bound protein was eluted in one step with 50 mM Tris pH=8.0, 500 mM CaCl₂) and all fractions with absorbance [A₂₈₀]>500AU were pooled together. The Q-elution fraction (the eluate) was then used for further fractionation by size exclusion chromatography.

### Size Exclusion Chromatography of Silk.

The Low Skid silk eluate fraction of the Q-Sepharose anion exchange chromatography (Q-elution) was the starting material for size exclusion chromatography. The eluate was loaded onto a HiLoad 26/600 Superdex 200 μg gel filtration column for fractionation, using the AKTA Pure 25 L system. All buffers used during fractionation were filtered through 0.22 μm PES filter as well and were degassed. The Low Skid silk was loaded on the Superdex 200 gel filtration column, and was run with 50 mM Tris, 200 mM CaCl₂), pH=8.0, to fractionate the Q-elution Low Skid silk. The eluted silk compositions were collected in 10 ml fractions. Fractions 6-10 (AS77, AS78, AS79, AS80, AS81) were collected, and have relatively narrow range of molecular weight. The fractions were placed in 3.5 kDa cutoff dialysis bags, and were concentrated by covering the dialysis bags with polyethylene glycol 35000 Da. Then, fractions in the dialysis bags were immersed in 160× volumes of 50 mM Tris pH=8.0 overnight, and then were immersed in a new batch of 160× volume of 50 mM Tris pH=8.0. Samples were kept at 4° C. until they were used.

### Analytical/Protein Characterization Methods.

### Protein Concentration Determination.

Protein concentration was determined by absorbance at 220 nm or 280 nm. Solubilized silk preparations were diluted until A₂₈₀ was between 0.1-1. In this range the absorbance correlates linearly with the concentration of silk in the solution and the correlation is 1AU=1 mg/mL soluble silk proteins. Final concentrations in the initial silk solution were calculated after adjustment for the dilution used for the absorbance measurement.

### Analytical Size Exclusion Chromatography.

Analytical Size Exclusion Chromatography is performed as described in detail in below Example 23. Analysis was performed in a PolySep GFC P-4000 LC Column, 300 mm×7.8 mm connected to an Agilent 1260 Infinity II HPLC system with an Agilent G7162A RID Refractive Index Detector. The mobile phase used for the analysis was a solution of 0.1M NaCl, 12.5 mM Na₂HPO₄, pH 7 (the pH was adjusted with phosphoric acid and filtered through a 0.2 μm PES filter into a clean glass media bottle). 25 μL of sample were loaded on the column and the analysis was performed at 25° C. with a flow rate of 1 mL/min for 20 min. Calculation of the molecular weight of each sample was done using Agilent Technologies Open LAB CDS ChemStation Edition for LC & LC/MS Systems software Cirrus SEC data collection and molecular weight analysis software.

### SDS Polyacrylamide gel.

Low Skid silk fractions were uploaded onto a Mini-Protean TGX precast gel, 4-20%, with a protein marker Trident Prestained Protein Ladder for molecular weight reference. The SDS polyacrylamide gel was stained using ReadyBlue™ Protein stain gel. Gels were immersed in ReadyBlue™ solution for 1 h, then destained with DI/RO water.

### Self-Assembly Assay.

The silk Self Assembly Assay (SAF) was performed in 35% v/v 2-propanol and 50 mM CH₃COONa pH=5. Each reaction was done in a final volume of 200 μL. Total silk protein concentration was 5 mg/mL. First the buffer of 50 mM CH₃COONa pH=5.0, 35% v/v 2-propanol was prepared. Then DI/RO water was added so that after the addition of the volume of silk protein required to reach a final concentration of 5 mg/mL the total volume would be 200 μL. The protein was added last and mixed with very gentle pipetting to reduce shearing force. The protein mixtures were placed in wells of flat-bottom 96-well plates and a layer of 100 μL of Mineral Oil carefully, so as to not create any bubbles. Absorbance was recorded at 550 nm for 24h. Recorded values were exported in Excel files for storage and further analysis.

### Dynamic Light Scattering Analysis of Silk Compositions.

Low Skid silk compositions were diluted to a concentration of 1 mg/mL and filtered with a 0.22 μm PES syringe filter. All measurements were performed with a Malvern Zetasizer Pro Red Label, detection angle of 173°. The Red Label system operates with a 10 mW He—Ne laser (633 nm). The software used is ZS XPLORER version 3.2.1.11. All measurements were done with 4.2 ml polystyrol/polystyrene transparent cuvettes. samples were measured at 25° C., with 120 sec of equilibration time. The intensity size distributions, autocorrelation, and Z-average were measured.

TABLE 6
Z-average of AS77-AS81 calculated by Dynamic Light Scattering.

| Tables | |
|---|---|
| Low Skid silk/modified | Z-average (d · |
| polypeptide composition | nm) |
| AS77 | 17.16 |
| AS78 | 15.14 |
| AS79 | 14.27 |
| AS80 | 13.07 |
| AS81 | 12.19 |

The Z-average value of each silk/modified polypeptide composition was calculated by the Zetasizer Pro. Shown here are the Z-average values of each silk composition. The abbreviation d. nm refers to the diameter in nanometers.

TABLE 7

| Low Skid silk/modified | | |
|---|---|---|
| polypeptide composition | Mw | PDI |
| AS77 | 55714 | 1.1836 |
| AS78 | 45350 | 1.1725 |
| AS79 | 38633 | 1.2549 |
| AS80 | 34476 | 1.2366 |
| AS81 | 28750 | 1.3648 |
| Low Skid silk | 19500 | 2.2 |

Molecular weight (Mw) and Polydispersity (PDI) values of silk compositions AS77-AS81. Silk/modified polypeptide compositions AS77, AS78, AS79, AS80, and AS81 were analyzed by size exclusion chromatography (SEC) column with HPLC, and values of molecular weights (Mw) and Polydispersity (PDI) are indicated.

### Example 7. Low Skid Silk/Modified Polypeptide Compositions Isolated by Size Properties

Described herein is a novel method to generate compositions of polypeptide that are derived from B. mori silkworm cocoons and comprise of natural and modified polypeptides. This novel composition is called Low Skid silk/modified polypeptide compositions.

The novel production method involves removing sericin through several washing steps with an organic sodium carbonate salt with tightly controlled multi-stage temperature cycles and agitation as the first step in forming natural/modified polypeptide composition. Next the silk is dried to remove remaining water at controlled temperature to maintain polypeptide composition. The silk is then dissolved in high concentration of Lithium salt at 125° C. for 6 hours to achieve the compositions of Low Skid silk. The liquid solution is then filtered and purified to remove the Lithium salt leaving only the natural/modified silk compositions in solution with pure water.

Low Skid silk/modified polypeptide compositions comprise of populations of silk/modified polypeptides with distinctive properties.

Low Skid silk/modified polypeptide composition does not self-assemble at 5 mg/mL. Low Skid silk/modified polypeptide composition comprises of a variety of populations of silk/modified polypeptides; here distinct populations were isolated based on size, by fractionating Low Skid silk/modified polypeptides by size exclusion chromatography.

A high-resolution separation of five silk compositions—AS82, AS83, AS84, AS85, and AS86 was achieved. These silk compositions differ from one another by their average size, when AS82 is the largest, and AS86 is the smallest. These silk compositions do not self-assemble under conditions that promote self-assembly at 5 mg/mL.

In addition to the well-separated silk compositions, lower-molecular-weight silk compositions (AS87, AS88, AS89) were generated that are less well-resolved, but composed of significantly smaller polypeptide populations. These silk compositions self-assemble into a gel within a few days under conditions that promote self-assembly at 5 mg/mL, but not as quickly as Mid Skid silk (starting to self-assemble within 3 h).

### Low Skid Silk/Modified Polypeptide Compositions Isolated by Size Properties.

The Low Skid silk/modified polypeptide compositions described in this invention are novel compositions of silk and modified polypeptides composed of a variety of silk polypeptide populations, generated by the exclusive treatment method of natural silk produced by B. mori. These silk compositions contain modified amino acid sequences that result from the silk processing method and scale. The tight controls over temperature, silk concentration, buffers and salt concentrations, physical agitation, and purification allow us to precisely develop silk compositions with a variety of performance criteria. Isolation of these populations by size reveals different characteristics, like high solubility and stability in solution over time in some populations, and the tendency to self-assemble in others. the purification method allows us to isolate silk/modified polypeptide compositions that display biological activities and could be used for therapeutic purposes.

Silk is a complex natural biomaterial that has the potential to be utilized in various applications such as the development of implantable medical devices, and the development of soluble polypeptide compositions of medical value. Additionally, it was demonstrated that silk peptides have anti-genotoxic effects. However, silk, in its natural form, is not soluble, and silk polypeptide compositions, without the proper processing, display poor solubility in solution and tend to self-assemble and aggregate over time. The kinetics of this self-assembly is unpredictable, and highly depends on the composition of the silk polypeptides/modified composition. Novel silk/modified polypeptide compositions were produced and specific populations were isolated within these compositions. The isolation process allows us to control the properties of the silk compositions and develop products with predictable and desired characteristics.

### Generation of Low Skid Silk/Modified Polypeptide Compositions.

Silk is washed to remove sericin at 100 C and 60 C with sodium carbonate and then dried at 60 C. The silk is then dissolved in 9.3M Lithium Bromide at 125 C for 6 hours. This dissolution step controls not only molecular weight but also the polypeptide modifications creating the natural/modified silk compositions. The silk is then filtered to remove undissolved debris and purified using 10 kDa cutoff PES hollow fiber membranes and concentrated using the same process leaving only natural/modified silk composite in solution with pure water. Every unit ops is tightly controlled for temperature, time, concentrations, agitation, and shear.

### Isolation of Low Skid/Modified Polypeptide Compositions

### Isolation of the AS82-AS89 Silk/Modified Polypeptide Composition Component of Low Skid Silk/Modified Polypeptide Composition.

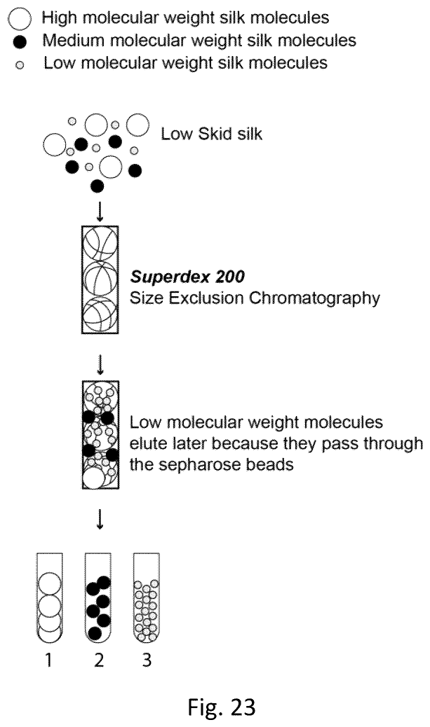

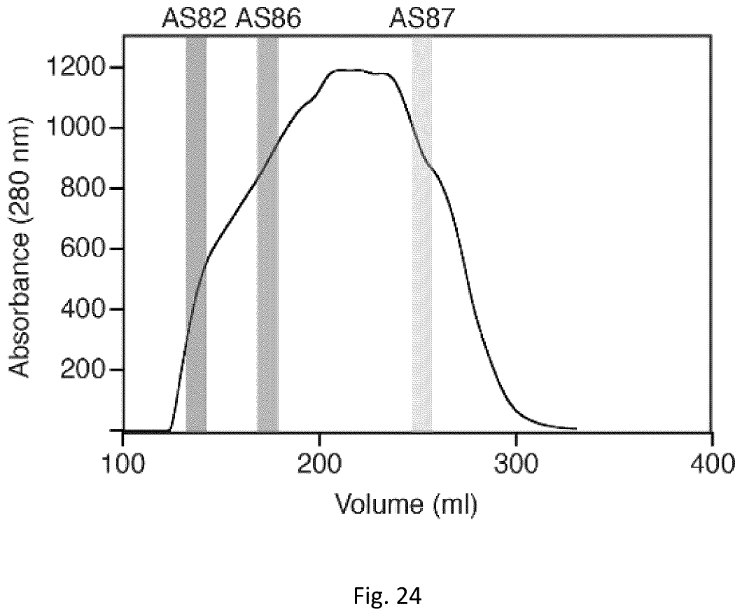

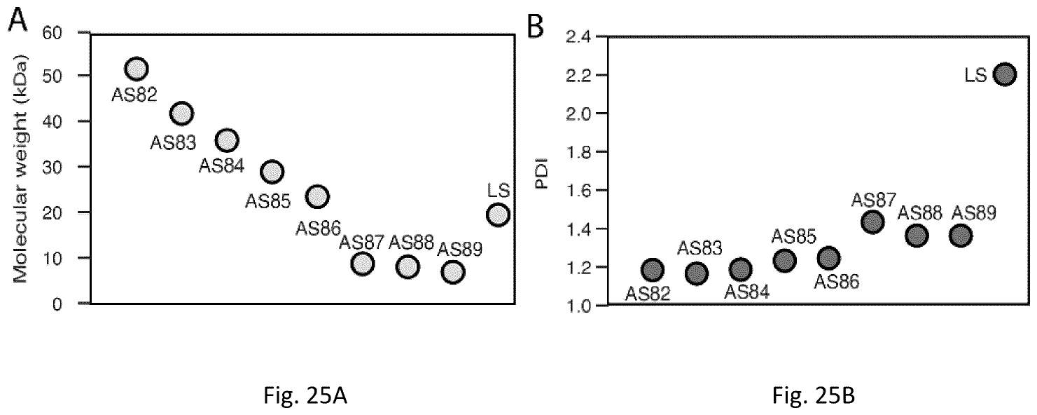

To isolate AS82-AS89 Low Skid silk/modified polypeptide compositions were fractioned using HiLoad 26/600 Superdex 200 μg size exclusion chromatography column (FIGS. 23 & 24). Tris was added to the silk preparations to a final concentration of 50 mM Tris-HCl, pH=8.0. The silk was centrifuged and filtered before loading to the HiLoad 26/600 Superdex 200 μg column, to remove any preformed aggregates. The silk compositions were fractionated by the HiLoad 26/600 Superdex 200 μg, where the largest polypeptide compositions were eluted first, and each following fraction had a population of lower molecular weight of silk compositions (FIG. 24). Low Skid silk preparation solutions have a characteristic yellow hue, and the fractionated silk compositions had a yellow hue. When silk formulations AS82-AS89 are analyzed with an analytical SEC column (see materials and methods) with HPLC, each of the silk formulations demonstrates a different average Mw, and a different Polydispersity (PDI) value (FIG. 24, Table 9). In general, AS82 has the highest Mw (51936 Da), while AS89 has the lowest Mw (6826 Da). The PDI values display a differential change as well. The PDI value of AS82 is relatively low (1.1738), and AS89 is higher (1.3544) (FIGS. 25A-25B, Table 9).

AS82-AS86 Silk Compositions Demonstrate Relative Uniformity by Dynamic Light Scattering, while AS87-AS89 Silk Compositions Contain Multiple Peptide Populations Sizes.

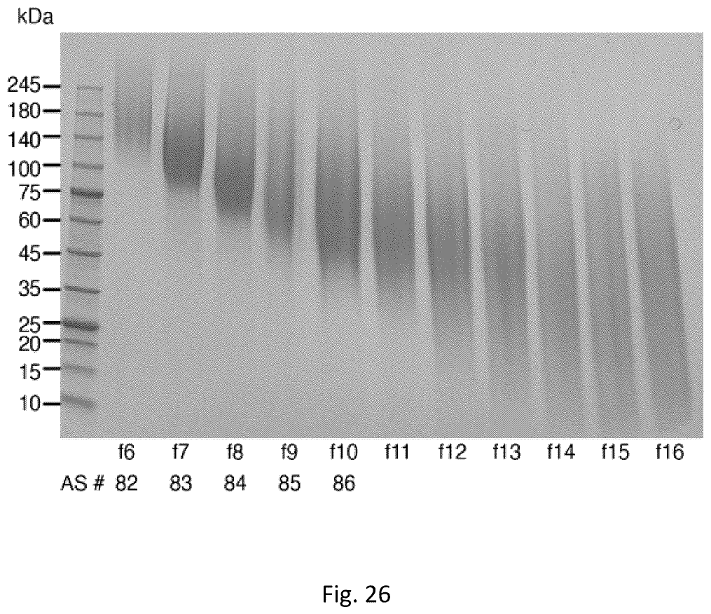

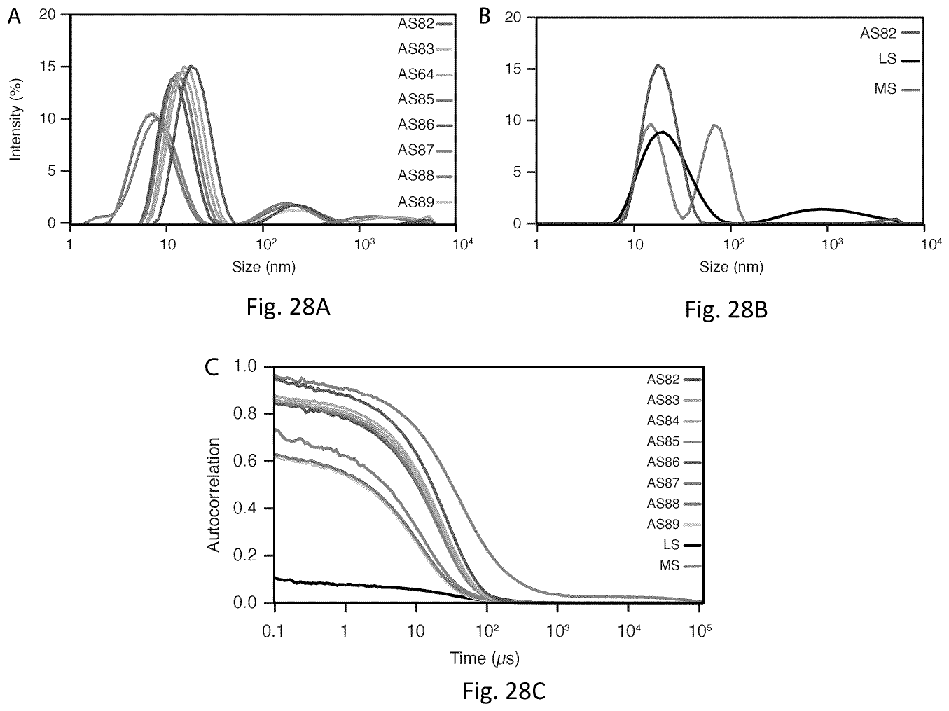

In dynamic light scattering analysis (Zetasizer Pro, FIGS. 28A-28C, Table 8), AS82-AS86 demonstrated relatively uniform, though broad, peaks where AS82 has the largest Z-average (18.174 nm), then AS83 (15.659 nm), and so on (Table 9). AS87, AS88, and AS89 are of lower molecular weight, and are eluted later during the chromatography, where the resolution of the Superdex 200 is not optimal and cannot resolve the peptide populations very well (the column resolution is reduced for proteins smaller than average size of ˜44 kDa), as can be observed by SDS gel electrophoresis in FIG. 26 (fraction 11 and on). Dynamic light scattering shows two peaks for these fractions, indicating the presence of several populations (FIG. 28A).

Self-Assembly of Low and Mid Skid/Modified Polypeptide Compositions Self-Assembly Assay and Data Derived from it

To study the stability of silk/modified peptide compositions in solution Self-Assembly assays were performed at a concentration of 5 mg/mL.

The absorbance at 550 nm curves of the self-assembly assays are sigmoid and they can be described as logistic curves. The typical logistic function is:

$$f ( x ) = \frac{{A}_{max}}{1 + {e}^{- k ( t - t 0.5 )}}$$

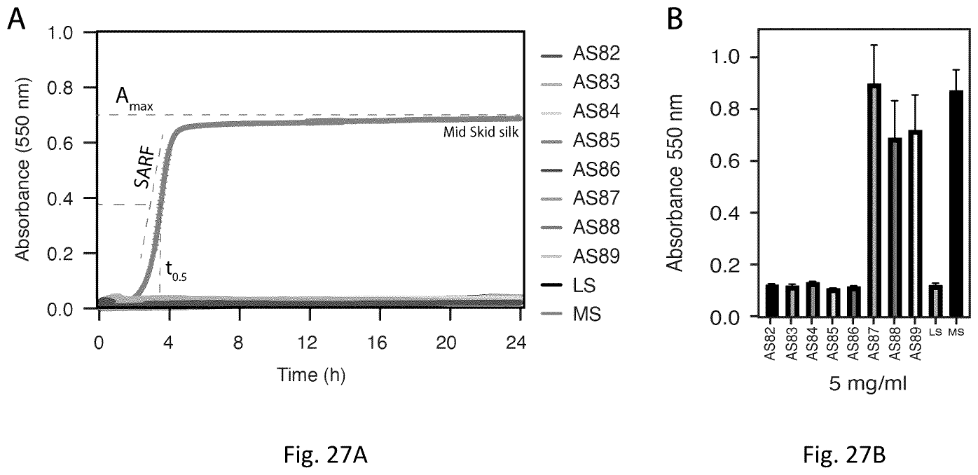

Aₘₐₓ is the maximum density of the gel formed
k is the Self-Assembly Rate Factor (SARF)
t_{0.5} is the time point at which 50% of the gel has formed
e is the exponential equation for the specific curve
(see FIG. 27A the red dotted lines for a better demonstration of how these factors from the Self-Assembly experiments were calculated)

Another parameter that was introduced to characterize the propensity of silk to form gels is the Self-Assembly Factor (F_{SAF}) which is:

$${F}_{SAF} = \frac{1}{t 0.5 ( min )} * {A}_{max} ( Abs ) * 1000$$

Using the experimental data from the Self-Assembly assays that were performed with the various novel isolated silk polypeptides, these parameters were calculated and used to dissect their properties (FIGS. 27A-27B). Four parameters were focused on, collectively referred to as Self-Assembly kinetic factors; the Self-Assembly Rate Factor (SARF), Aₘₐₓ, t0.5, and the Self-Assembly Factor (SAF) (FIGS. 27A-27B). The SARF shows how fast silk self-assembles to form gel after the reaction begins or the gelation nuclei have formed; Amax shows how dense is the gel that is formed after self-assembly is complete, t0.5 shows how long it takes for the self-assembly reaction to reach the point where gel density is and SAF shows the propensity of silk to self-assemble (FIGS. 27A-27B).

$$\frac{{A}_{max}}{2}$$

### AS82-AS86 Silk Compositions do not Self-Assemble.

Self-assembly assays revealed that Low Skid silk/modified peptide compositions do not self-assemble under the experimental system conditions (FIG. 27A). No self-assembly occurred after 24 hours, and no self-assembly was detected even 18 days post-assay (FIG. 27B). Mid Skid/modified peptide compositions was used as a positive control and shows fast self-assembly kinetics (FIGS. 27A-27B).

AS87-AS89 Silk Compositions Self-Assemble within Few Days.

Self-assembly assays as described before, revealed that AS87-AS89 do not self-assemble within 24 hours (FIG. 27A). However, if left for an extra 4-5 days, self-assembly occurs in these silk/modified polypeptide compositions (FIG. 27B). Mid Skid/modified peptide compositions was used as a positive control and shows fast self-assembly kinetics (FIGS. 27A-27B).

### Materials and Methods Used for the Generation and Characterization of AS82-AS89.

### Size Exclusion Chromatography of Silk.

The starting material, Low Skid silk at a concentration of 60 mg/mL, was provided by the manufacturing team. The Low Skid silk was transferred to 50 mM Tris, pH=8.0 buffer, and centrifuged at 16000 rpm, at 4° C., for 30 min to separate formed aggregates from soluble silk. The supernatant was collected and filtered through a 0.22 μm PES filter. Then, the silk was loaded onto a HiLoad 26/600 Superdex 200 μg gel filtration column for fractionation, using the AKTA Pure 25 L system. All buffers used during fractionation were filtered through 0.22 μm PES filter as well and were degassed. The Low Skid silk was loaded on the Superdex 200 gel filtration column, and was run with 50 mM Tris, 200 mM CaCl₂), pH8, to fractionate the Low Skid silk. The eluted silk compositions were collected in 10 ml fractions. Fractions 6-10 (AS82, AS83, AS84, AS85, AS86) were collected, and have relatively narrow range of molecular weight, while fractions 18-20 (AS87, AS88, AS89) have a broader molecular weight range, since these molecular sizes are outside of the HiLoad 26/600 Superdex 200 μg separation range. The fractions were placed in 3.5 kDa cutoff dialysis bags, and were concentrated by covering the dialysis bags with polyethylene glycol 35000 Da. Then, fractions in the dialysis bags were immersed in 160× volumes of 50 mM Tris pH=8.0 overnight, and then were immersed in a new batch of 160× volume of 50 mM Tris pH=8.0. Samples were kept at 4° C. until they were used.

Analytical/Protein Characterization methods.
Protein concentration determination.

Protein concentration was determined by absorbance at 220 nm or 280 nm. Solubilized silk preparations were diluted until A₂₈₀ was between 0.1-1. In this range the absorbance correlates linearly with the concentration of silk in the solution and the correlation is 1AU=1 mg/mL soluble silk proteins. Final concentrations in the initial silk solution were calculated after adjustment for the dilution used for the absorbance measurement.

### Analytical Size Exclusion Chromatography.

Analytical Size Exclusion Chromatography is performed as described in detail in Example 23. Analysis was performed in a PolySep GFC P-4000 LC Column, 300 mm×7.8 mm connected to an Agilent 1260 Infinity II HPLC system with an Agilent G7162A RID Refractive Index Detector. The mobile phase used for the analysis was a solution of 0.1M NaCl, 12.5 mM Na₂HPO₄, pH 7 (the pH was adjusted with phosphoric acid and filtered through a 0.2 μm PES filter into a clean glass media bottle). 25 μL of sample were loaded on the column and the analysis was performed at 25° C. with a flow rate of 1 mL/min for 20 min. Calculation of the molecular weight of each sample was done using Agilent Technologies Open LAB CDS ChemStation Edition for LC & LC/MS Systems software Cirrus SEC data collection and molecular weight analysis software.

### SDS Polyacrylamide Gel.

Low Skid silk fractions were uploaded onto a Mini-Protean TGX precast gel, 4-20%, with a protein marker Trident Prestained Protein Ladder for molecular weight reference. The SDS polyacrylamide gel was stained using ReadyBlue™ Protein stain gel. Gels were immersed in ReadyBlue™ solution for 1 h, then destained with DI/RO water.

### Self-Assembly Assay.

The silk Self Assembly Assay (SAF) was performed in 35% v/v 2-propanol and 50 mM CH₃COONa pH=5. Each reaction was done in a final volume of 200 μL. Total silk protein concentration was 5 mg/mL. First the buffer of 50 mM CH₃COONa pH=5, 35% v/v 2-propanol was prepared. Then DI/RO water was added so that after the addition of the volume of silk protein required to reach a final concentration of 5 mg/mL the total volume would be 200 μL. The protein was added last and mixed with very gentle pipetting to reduce shearing force. The protein mixtures were placed in wells of flat-bottom 96-well plates and a layer of 100 μL of Mineral Oil carefully so as to not create any bubbles. Absorbance was recorded at 550 nm for 24h.

### Dynamic Light Scattering Analysis of Silk Compositions.

Low Skid silk compositions were diluted to a concentration of 1 mg/mL and filtered with a 0.22 μm PES syringe filter. All measurements were performed with a Malvern Zetasizer Pro Red Label, detection angle of 173°. The Red Label system operates with a 10 mW He—Ne laser (633 nm). The software used is ZS XPLORER version 3.2.1.11. All measurements were done with 4.2 mL polystyrol/polystyrene transparent cuvettes. samples were measured at 25° C., with 120 sec of equilibration time. The intensity size distributions, autocorrelation, and Z-average were measured.

### Tables

TABLE 8
Z-average of AS82-AS89 calculated by Dynamic Light Scattering.

| Low Skid silk/modified polypeptide | Z-average (d · |
|---|---|
| composition | nm) |
| AS82 | 18.147 |
| AS83 | 15.659 |
| AS84 | 14.836 |
| AS85 | 13.473 |
| AS86 | 13.467 |
| AS87 | 8.577 |
| AS88 | 7.843 |
| AS89 | 7.681 |

The Z-average value of each silk/modified polypeptide composition was calculated by the Zetasizer Pro. Shown here are the Z-average values of each silk composition.

TABLE 9
Molecular weight (Mw) and Polydispersity (PDI)
values of silk compositions AS82-AS89.

| Low Skid silk/modified polypeptide | | |
|---|---|---|
| composition | Mw | PDI |
| AS82 | 51936 | 1.1738 |
| AS83 | 42028 | 1.1543 |
| AS84 | 36098 | 1.1755 |
| AS85 | 29089 | 1.2223 |
| AS86 | 23574 | 1.235 |
| AS87 | 8668 | 1.425 |
| AS88 | 7972 | 1.3537 |
| AS89 | 6826 | 1.3544 |
| Low Skid silk | 19500 | 2.2 |

Silk/modified polypeptide compositions AS82, AS83, AS84, AS85, AS86, AS87, AS88, and AS89 were analyzed by size exclusion chromatography (SEC) column with HBPLC, and values of molecular weights (Mw) and Polydispersity (PDI) are indicated.

### Example 8. Low Skid Silk/Modified Polypeptide Compositions Isolated by Charge. Hydrophobicity Levels, and Size Properties

Described herein is a novel method to generate compositions of polypeptide that are derived from B. mori silkworm cocoons and comprise of natural and modified polypeptides. This novel composition is called Low Skid silk/modified polypeptide compositions.

The novel production method involves removing sericin through several washing steps with an organic sodium carbonate salt with tightly controlled multi-stage temperature cycles and agitation as the first step in forming natural/modified polypeptide composition. Next the silk is dried to remove remaining water at controlled temperature to maintain polypeptide composition. The silk is then dissolved in high concentration of Lithium salt at 125° C. for 6 hours to achieve the compositions of Low Skid silk. The liquid solution is then filtered and purified to remove the Lithium salt leaving only the natural/modified silk compositions in solution with pure water.

Low Skid Silk/Modified Polypeptide Compositions Comprise of Populations of Silk/Modified Polypeptides with Distinctive Properties

Low Skid silk/modified polypeptide composition does not self-assemble at 5 mg/mL. Low Skid silk/modified polypeptide composition comprises of a variety of populations of silk/modified polypeptides; here it was sought to isolate distinct populations based on charge, hydrophobicity, and size, by fractionating Low Skid silk/modified polypeptides by anion exchange chromatography, followed by hydrophobic interaction chromatography and size exclusion chromatography. A high-resolution separation of ten distinct Low Skid silk/modified polypeptides compositions was achieved: five are negatively-charged silk compositions that display hydrophobicity characteristics as well—AS90, AS91, AS92, AS93, and AS94. These silk compositions differ from one another by their average size, when AS90 is the largest, and AS94 is the smallest. These silk compositions do not self-assemble under conditions that promote self-assembly at 5 mg/mL.

Additional five compositions are negatively charged silk compositions as well, that are less hydrophobic compared to AS90-AS94, and have relatively lower molecular weights (AS95, AS96, AS97, AS98, AS99, AS100). These silk compositions differ from one another by their average size, when AS95 is the largest, and AS100 is the smallest. These silk compositions may have the tendency to aggregate in solution, as can be demonstrated by dynamic light scattering and loss of material after filtration with 0.2 μm PES filter.

The Low Skid silk/modified polypeptide compositions described in this invention are novel compositions of silk and modified polypeptides composed of a variety of silk polypeptide populations, generated by the exclusive treatment method of natural silk produced by B. mori. These silk compositions contain modified amino acid sequences that result from the silk processing method and scale. The tight controls over temperature, silk concentration, buffers and salt concentrations, physical agitation, and purification allow us to precisely develop silk compositions with a variety of performance criteria. Isolation of these populations by charge, hydrophobicity, and size reveals new characteristics, like high solubility and stability in solution over time in some populations, and the tendency to aggregate in others. the purification method allows us to isolate silk/modified polypeptide compositions that display biological activities and could be used for therapeutic purposes.

Silk is a complex natural biomaterial that has the potential to be utilized in various applications such as the development of implantable medical devices, and the development of soluble polypeptide compositions of medical value. Additionally, it was demonstrated that silk peptides have anti-genotoxic effects However, silk, in its natural form, is not soluble, and silk polypeptide compositions, without the proper processing, display poor solubility in solution and tend to self-assemble and aggregate over time. The kinetics of this self-assembly is unpredictable, and highly depends on the composition of the silk polypeptides/modified composition. Novel silk/modified polypeptide compositions were produced and specific populations were isolated within these compositions. The isolation process allows us to control the properties of the silk compositions and develop products with predictable and desired characteristics.

### Generation of Low Skid Silk/Modified Polypeptide Compositions.

Silk is washed to remove sericin at 100° C. and 60° C. with sodium carbonate and then dried at 60° C. The silk is then dissolved in 9.3M Lithium Bromide at 125° C. for 6 hours.

This dissolution step controls not only molecular weight but also the polypeptide modifications creating the natural/modified silk compositions. The silk is then filtered to remove undissolved debris and purified using 10 kDa cutoff PES hollow fiber membranes and concentrated using the same process leaving only natural/modified silk composite in solution with pure water. Every unit ops is tightly controlled for temperature, time, concentrations, agitation, and shear.

### Isolation of Low Skid/Modified Polypeptide Compositions

### Isolation of the AS90-AS100 Silk/Modified Polypeptide Composition Component of Low Skid Silk/Modified Polypeptide Composition.

To isolate AS90-AS100 Low Skid silk/modified polypeptide compositions were fractioned using anion exchange chromatography (Q-Sepharose chromatography), following hydrophobic interactions (HIC) chromatography (Butyl ImpRes colum), followed by HiLoad 26/600 Superdex 200 μg size exclusion chromatography of the Q-HIC-eluate (AS90-94) and of the Q-HIC-flowthrough (AS95-100) (FIG. 1, 2). Prior to chromatography, Tris was added to the silk preparations to a final concentration of 50 mM Tris-HCl, pH=8.0. The silk was centrifuged and filtered before loading to the Q-Sepharose column, to remove any preformed aggregates. The silk compositions were loaded onto the Q-Sepharose column, and the flowthrough fraction was collected. The negatively charged silk compositions were eluted using high salt buffer (50 mM Tris, 500 mM CaCl₂)) (FIG. 2 A). The eluted fractions were pulled together and are referred to as the Q-elution fraction. The Q-flowthrough fraction is colorless and tends to aggregate.

The Q-elution was further fractionated by using a Butyl ImpRes column (FIG. 2 B), which separates polypeptides based on hydrophobicity. The chromatography was performed in the presence of 300 mM ammonium sulfate [(NH₄)₂SO₄], to expose hydrophobic regions within the silk polypeptides. The highly charged flowthrough fraction (Q-HIC-flowthrough) was collected for further fractionation by size exclusion chromatography. The more hydrophobic, bound silk peptides were eluted using 50 mM Tris, pH=8.0 in the absence of ammonium sulfate, to reverse the exposure of the hydrophobic regions in silk polypeptides, which results in their release from the Butyl ImpRes column.

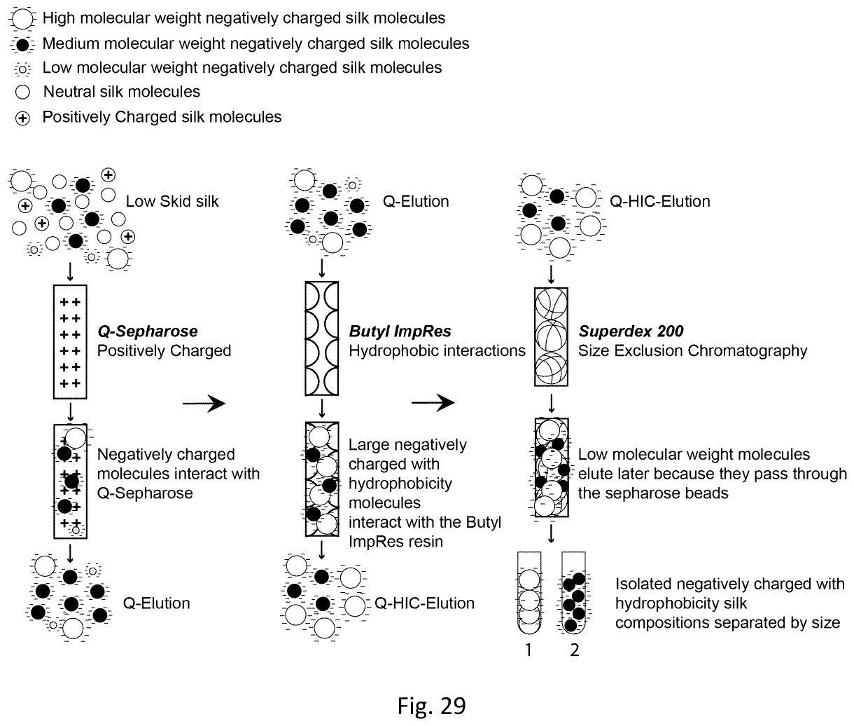

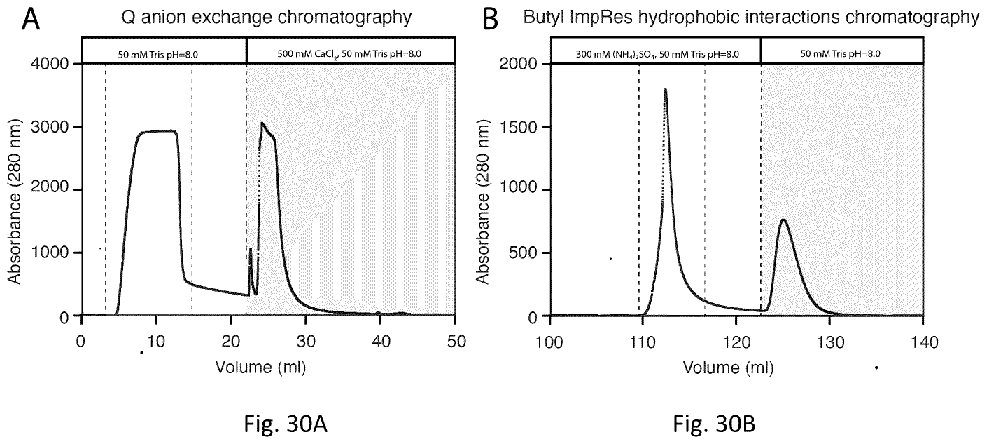

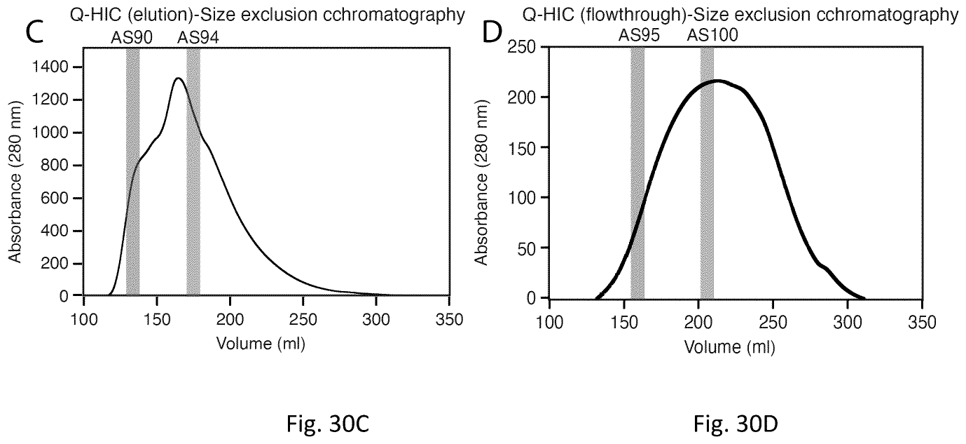

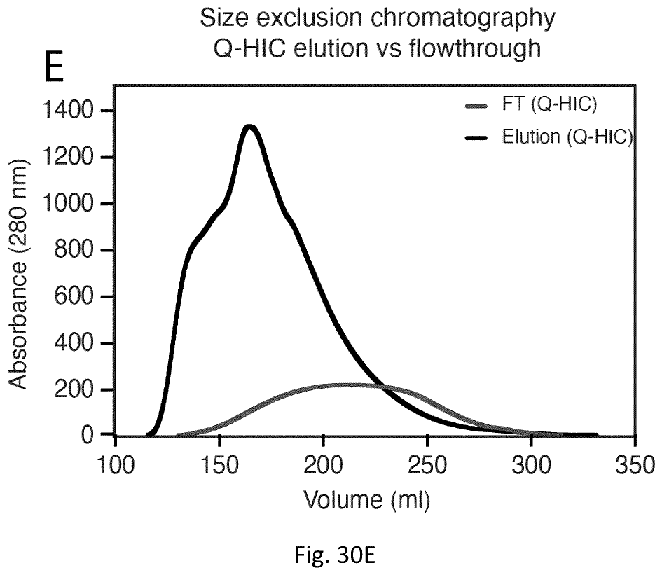

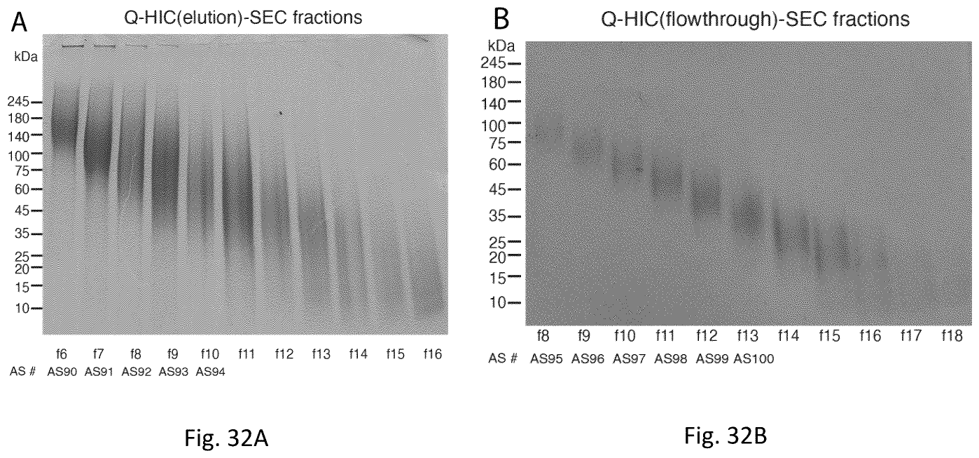

The Q-HIC-elution was further fractionated by the HiLoad 26/600 Superdex 200 μg, where the largest polypeptide compositions were eluted first, and each following fraction had a population of lower molecular weight of silk compositions (FIG. 29, FIGS. 30C and 30D). The fractions that were isolated are AS90-AS94. Then, the Q-HIC-flowthrough fraction was fractionated as well by the HiLoad 26/600 Superdex 200 μg, resulting in the generation of AS95-AS100. Comparing the size exclusion chromatograms of Q-HIC(elution) and Q-HIC(flowthrough) (FIG. 30E), it is evident that the Q-HIC(elution) fraction is composed of higher-molecular-weight peptide composition, while the silk peptides that compose the Q-HIC(flowthrough) fraction are eluted later, indicating smaller molecular weights (Also see FIGS. 32A and 32B for SDS-PAGE of fractions AS90-AS94 (32A) and AS95-AS100 (32B)).

Low Skid silk preparation solutions have a characteristic yellow hue. The Q-elution and the Q-HIC-elution fractions had a strong yellow hue, while the Q-flowthrough fraction is transparent, and tends to self-assemble very quickly. The Q-HIC-elution silk compositions that are fractionated by size exclusion (AS90-94) also had a yellow hue. The Q-HIC-flowthrough fractions that were fractionated by size exclusion chromatography (AS95-AS100) were colorless.

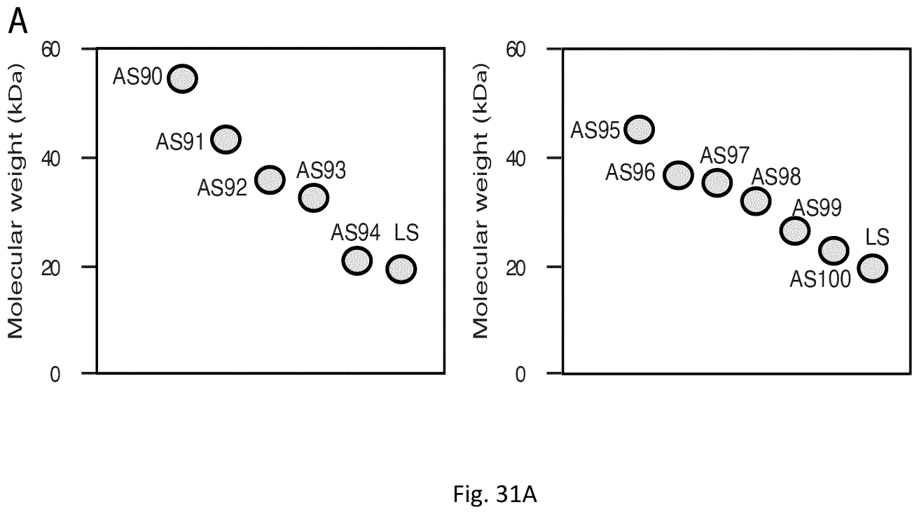

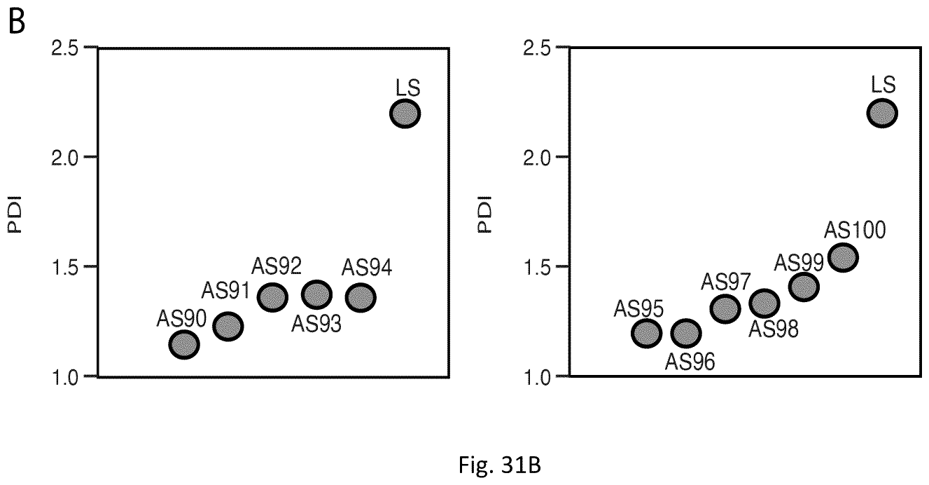

When silk formulations AS90-AS94 and AS95-AS100 are analyzed with an analytical SEC column (see materials and methods) with HPLC, each of the silk formulations demonstrates a different average Mw, and a different Polydispersity (PDI) value (FIGS. 31A-31B, Table 11). Among the Q-HIC-elution fractions, AS90 has the highest Mw (54620 Da), while AS94 has the lowest Mw (21057 Da). The PDI values display a differential change as well. The PDI value of AS90 is the lowest (1.1487), and AS94 is higher (1.3615) (FIGS. 31A-31B, Table 11). The Q-HIC-flowthrough fraction is composed of a smaller population of silk peptides, where the first eluted fraction, AS95, has the higher molecular weight among these fractions (45262 Da), 10 kDa smaller than the first eluted fraction of the Q-HIC-elution fraction (AS90). AS100 has the lowest molecular weight among the Q-HIC-flowthrough fractions (22799 Da). In general, AS95-AS100 demonstrate a trend of higher polydispersity compared to AS90-AS94. The PDI value of AS95 is the lowest (1.1988), and AS100 is higher (1.5438) (FIGS. 31A-31B, Table 11).

Unfractionated Low Skid silk has an average Mw of ˜19500, indicating that most of the peptide population tends to have lower molecular weight than fractions AS90-AS100. The polydispersity of unfractionated Low Skid silk is ˜2.2—significantly higher than the values of fractions AS90-AS100. This suggests that the unfractionated Low Skid silk is composed of a much diverse peptide population compared to fractions AS90-AS100.

AS90-AS94 Silk Compositions Demonstrate Relative Uniformity by Dynamic Light Scattering and Show Gradual Particle Size Distribution.

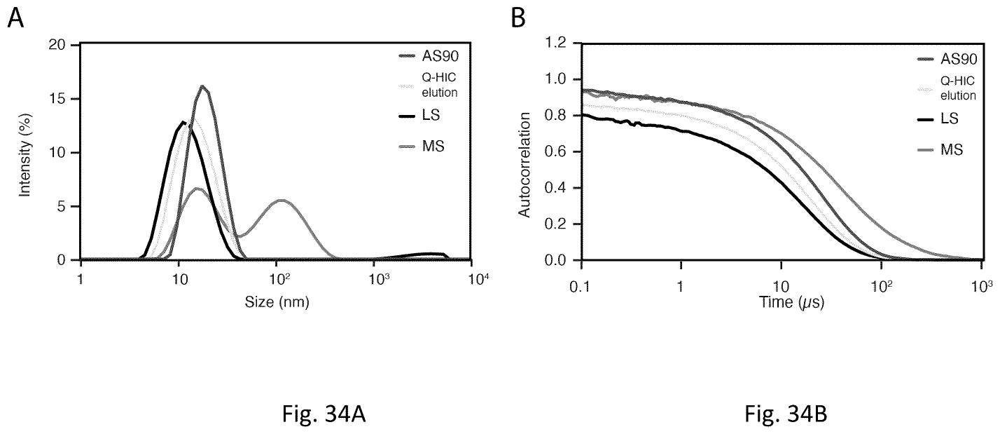

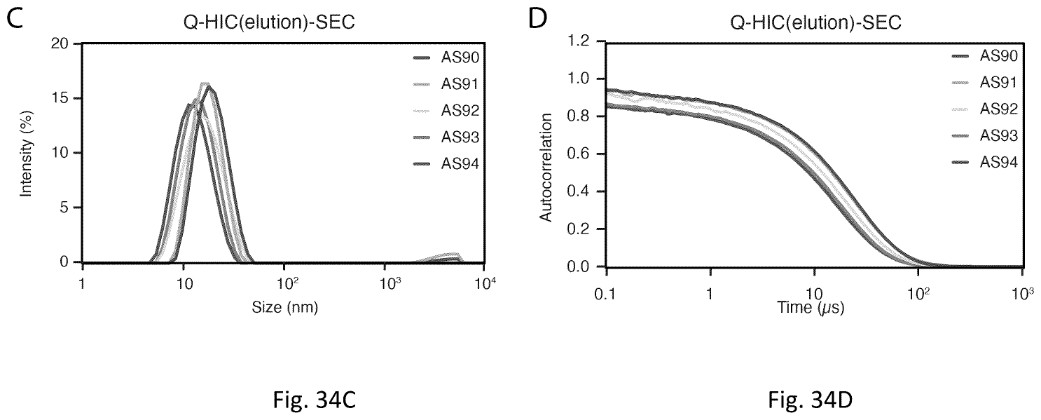

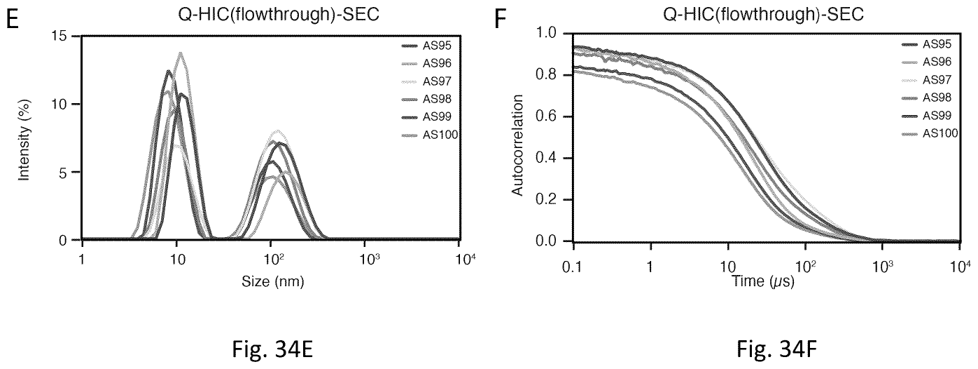

In dynamic light scattering analysis (Zetasizer Pro, FIGS. 34A-34F, Table 1), AS90-AS94 demonstrated relatively uniform, though broad, peaks where AS90 has the largest Z-average (17.617 d. nm), then AS91 (16.803 d. nm), and so on (Table 10), demonstrating the efficiency of the fractionation by size of the Q-HIC-elution fraction (FIGS. 34C and 34D). Low Skid silk and Mid Skid silk show broad peaks (FIGS. 34A and 34B), while the AS90 fraction shows more uniformity and narrower peak compared to the two and to the Q-HIC elution fraction, from which AS90 is derived.

AS95-AS100 silk compositions display non-uniform silk compositions by dynamic light scattering. AS95-AS100 demonstrate by dynamic light scattering the presence of two major broad peaks for each fraction, indicating a wide range of size of peptide populations (FIGS. 34E and 34F). The Z-average values of AS95-AS100 are not in descending order like the Z-average of AS90-AS94 and are more variable (Table 10).

AS95-A100 fractions and Q-HIC (flowthrough) may have the tendency to aggregate. During the generation AS95-AS100 there has been an extensive loss of protein material. By following the UV280 detection of protein amount that was flowed through the Butyl ImpRes column and did not bind the column, it was expected to find a large amount of silk peptides in solution. However, during size-exclusion chromatography, the amount of silk peptides in the resulting fractions was low (FIG. 32B). It is hypothesized that a large portion of the silk peptides has aggregated or did not go through the filtration step of the Q-HIC(flowthrough), and was lost before loading onto the gel filtration column. SDS-PAGE of AS95-AS100 that was run immediately after size exclusion chromatography (FIG. 32B) showed distinct populations of silk peptides, in a descending order of molecular weight. However, dynamic light scattering analysis that was performed later showed diverse size populations. This can indicate an aggregation over time of silk peptides in fractions AS95-AS100.

Self-Assembly of Low and Mid Skid/Modified Polypeptide Compositions Self Assembly Assay and Data Derived from it

To study the stability of silk/modified peptide compositions in solution Self-Assembly assays were performed at a concentration of 5 mg/mL.

The absorbance at 550 nm curves of the self-assembly assays are sigmoid and they can be described as logistic curves. The typical logistic function is:

$$f ( x ) = \frac{{A}_{max}}{1 + {e}^{- k ( t - t 0.5 )}}$$

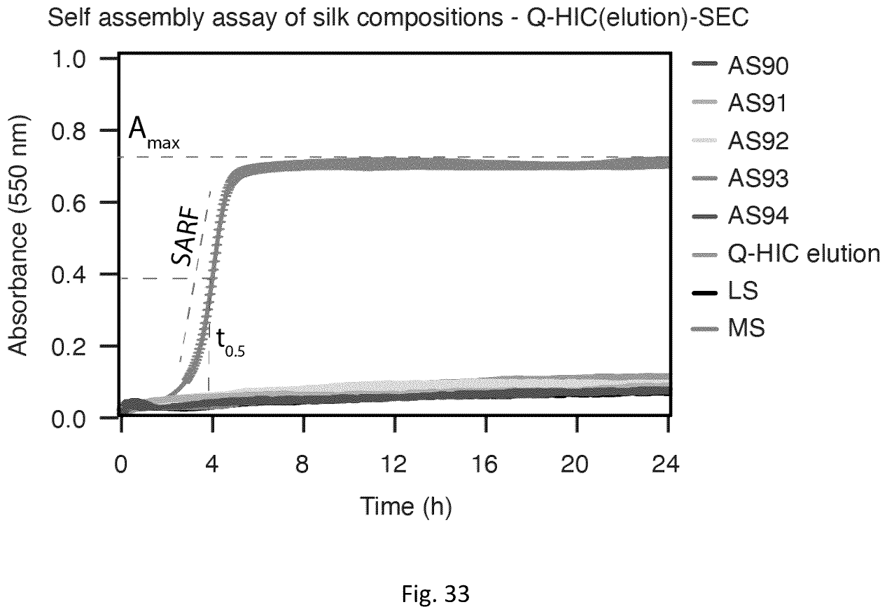

Aₘₐₓ is the maximum density of the gel formed
k is the Self-Assembly Rate Factor (SARF)
t_{0.5} is the time point at which 50% of the gel has formed
e is the exponential equation for the specific curve
(see FIG. 33 the red dotted lines for a better demonstration of how these factors from the Self-Assembly experiments are calculated)

Another parameter that was introduced to characterize the propensity of silk to form gels is the Self-Assembly Factor (F_{SAF}) which is:

$${F}_{SAF} = \frac{1}{t 0.5 ( min )} * {A}_{max} ( Abs ) * 1000$$

Using the experimental data from the Self-Assembly assays performed with the various novel isolated silk polypeptides, these parameters were calculated and used to dissect their properties (FIG. 33). Four parameters were focused on, collectively referred to as Self-Assembly kinetic factors; the Self-Assembly Rate Factor (SARF), Aₘₐₓ, t0.5, and the Self-Assembly Factor (SAF) (FIG. 33). The SARF shows how fast silk self-assembles to form gel after the reaction begins or the gelation nuclei have formed; Aₘₐₓ shows how dense is the gel that is formed after self-assembly is complete, t_{0.5} shows how long it takes for the self-assembly reaction to reach the point where gel density is

$$\frac{{A}_{max}}{2}$$

and SAF shows the propensity of silk to self-assemble (FIG. 33).

### AS90-AS94 Silk Compositions do not Self-Assemble.

Self-assembly assays revealed that Low Skid silk/modified peptide compositions do not self-assemble under the experimental system conditions (FIG. 33). Mid Skid/modified peptide compositions was used as a positive control and shows fast self-assembly kinetics (FIG. 33). Fractions AS95-AS100 were not tested for self-assembly since the purification process did not result in sufficient amount of silk peptides to perform the assay.

### Materials and Methods Used for the Generation and Characterization of AS90-AS100. Anion Exchange Chromatography of Silk.

Low Skid silk was provided by the manufacturing team at a concentration of 60 mg/mL. 50 mM Tris, pH=8.0 buffer was added to the Low Skid silk, and the silk was centrifuged at 16000 rpm (rotor JA-18, Beckman coulter, average of 28100×g), at 4° C., for 30 min to separate formed aggregates from soluble silk. The supernatant was collected and filtered through a 0.22 μm PES filter. For silk fractionation Q-Sepharose prepacked columns connected to an AKTA pure 25L or HiPrep Q FF 16/10 20 mL Column, or HiTrap™ Capto™ Q 1 mL column were used. All buffers used were filtered through a 0.22 μm PES filter and degassed with sonication. Centrifuged and filtered Low Skid silk was loaded on 5×5 mL HiTrap Q HP columns washed with 10 column volumes of 50 mM Tris pH=8.0, 10 column volumes of 50 mM Tris pH=8.0, 500 mM CaCl₂) and finally 10 column volumes of 50 mM Tris pH=8.0. 150 mL of centrifuged Low Skid silk were loaded on the column with a flow rate of 5 mL/min. The flow-through was collected. The column was washed with 50 mM Tris pH=8.0 until the absorbance at 280 nm [A₂₈₀] got to 100 AU. Bound protein was eluted in one step with 50 mM Tris pH=8.0, 500 mM CaCl₂) and all fractions with absorbance [A₂₈₀]>500AU were pooled together. This process was performed twice (total 300 ml of starting material of Low Skid silk was fractionated).The Q-elution fractions (the eluate) were then used for further fractionation by hydrophobic interactions chromatography.

### Hydrophobic Interactions Chromatography of Silk.

The buffer of Q-eluate was exchanged with water with dialysis in ×100 volumes of water. After the buffer exchange was complete, 50 mM Tris pH=8.0, 300 mM (NH₄)₂SO₄ were added to the Q-eluate. A column of Butyl ImpRes (Cytiva) resin was used for the creation of AS90-AS100. The Butyl ImpRes column was washed with ×10 column volumes of degassed and filtered (0.22 μm) 50 mM Tris pH=8.0, 300 mM (NH₄)₂SO₄, 10× column volumes of degassed and filtered (0.22 μm) 50 mM Tris pH=8.0 and 10× column volumes of degassed and filtered (0.22 μm) 50 mM Tris pH=8.0, 300 mM (NH₄)₂SO₄. The Q-eluate (200 mL) was used for the fractionation. Q-eluate silk was in 50 mM Tris pH=8.0, 300 mM (NH₄)₂SO₄ and loaded on the Butyl ImpRes column. All unbound proteins (the flowthrough fraction) were collected and saved for further analysis. After loading was complete, the Butyl ImpRes column was washed 10× column volumes of 50 mM Tris pH=8.0, 300 mM (NH₄)₂SO₄ until the OD280=about 100 AU. After the washing step was complete, bound silk molecules were eluted with 1.5× column volumes of 50 mM Tris pH=8.0. The elution was collected, and both Q-HIC(elution) and Q-HIC(flowthrough) fractions were transferred to dialysis bags and dialyzed against 3 mM Tris, pH=8.0. The two fractions were concentrated by covering the dialysis bags with polyethylene glycol 35000 Da and saved for further fractionation by size exclusion chromatography.

### Size Exclusion Chromatography of Silk.

Both the elution fraction of the hydrophobic interactions chromatography (Q-HIC-elution) and the flowthrough fraction (Q-HIC-flowthrough) were filtered (0.22 μm PES filter) to discard preformed aggregates, and fractionated separately by size exclusion chromatography. The eluate or the flowthrough fraction was loaded onto a HiLoad 26/600 Superdex 200 μg gel filtration column for fractionation, using the AKTA Pure 25 L system. All buffers used during fractionation were filtered through 0.22 μm PES filter and were degassed. The Q-HIC(elution) or Q-HIC(flowthrough) fractions were loaded on the Superdex 200 gel filtration column, and were run with 50 mM Tris, 200 mM CaCl₂), pH=8.0. The eluted silk compositions were collected in 10 ml fractions. Fractions 6-10 of the Q-HIC(elution) (AS90, AS91, AS92, AS93, AS94) were collected, and have relatively narrow range of molecular weight. Fractions 8-13 (AS95, AS96, AS97, AS98, AS99, AS100) were collected during the fractionation of Q-HIC(flowthrough). The fractions were placed in 3.5 kDa cutoff dialysis bags, and were concentrated by covering the dialysis bags with polyethylene glycol 35000 Da. Then, fractions in the dialysis bags were immersed in 160× volumes of 50 mM Tris pH=8.0 overnight, and then were immersed in a new batch of 160× volume of 50 mM Tris pH=8.0. Samples were kept at 4° C. until they were used.

### Analytical/Protein Characterization Methods.

### Protein Concentration Determination.

Protein concentration was determined by absorbance at 220 nm or 280 nm. Solubilized silk preparations were diluted until A₂₈₀ was between 0.1-1. In this range the absorbance correlates linearly with the concentration of silk in the solution and the correlation is 1AU=1 mg/mL soluble silk proteins. Final concentrations in the initial silk solution were calculated after adjustment for the dilution used for the absorbance measurement.

### Analytical Size Exclusion Chromatography.

Analytical Size Exclusion Chromatography is performed as described in detail in Example 23. Analysis was performed in a PolySep GFC P-4000 LC Column, 300 mm×7.8 mm connected to an Agilent 1260 Infinity II HPLC system with an Agilent G7162A RID Refractive Index Detector. The mobile phase used for the analysis was a solution of 0.1M NaCl, 12.5 mM Na₂HPO₄, pH 7 (the pH was adjusted with phosphoric acid and filtered through a 0.2 μm PES filter into a clean glass media bottle). 25 μL of sample were loaded on the column and the analysis was performed at 25° C. with a flow rate of 1 mL/min for 20 min. Calculation of the molecular weight of each sample was done using Agilent Technologies Open LAB CDS ChemStation Edition for LC & LC/MS Systems software Cirrus SEC data collection and molecular weight analysis software.

### SDS Polyacrylamide Gel.

Low Skid silk fractions were loaded onto a Mini-Protean TGX precast gel, 4-20%, with a protein marker Trident Prestained Protein Ladder for molecular weight reference. The SDS polyacrylamide gel was stained using ReadyBlue™ Protein stain gel. Gels were immersed in ReadyBlue™ solution for 1 h, then destained with DI/RO water.

### Self-Assembly Assay.

The silk Self Assembly Assay (SAF) was performed in 35% v/v 2-propanol and 50 mM CH₃COONa pH=5. Each reaction was done in a final volume of 200 μL. Total silk protein concentration was 5 mg/mL. First, the buffer of 50 mM CH₃COONa pH=5.0, 35% v/v 2-propanol was prepared. Then DI/RO water was added so that after the addition of the volume of silk protein required to reach a final concentration of 5 mg/mL the total volume would be 200 μL. The protein was added last and mixed with very gentle pipetting to reduce shearing force. The protein mixtures were placed in wells of flat-bottom 96-well plates and a layer of 100 μL of Mineral Oil carefully, so as to not create any bubbles. Absorbance was recorded at 550 nm for 24h.

Dynamic Light Scattering analysis of silk compositions.

Low and Mid Skid silk compositions were diluted to a concentration of 1 mg/mL and filtered with a 0.22 μm PES syringe filter. All measurements were performed with a Malvern Zetasizer Pro Red Label, detection angle of 173°. The Red Label system operates with a 10 mW He—Ne laser (633 nm). The software used is ZS XPLORER version 3.2.1.11. All measurements were done with 4.2 ml polystyrol/polystyrene transparent cuvettes. Samples were measured at 25° C., with 120 sec of equilibration time. The intensity size distributions, autocorrelation, and Z-average were measured.

TABLE 10
Z-average of AS90-AS100 calculated
by Dynamic Light Scattering.

| Low Skid silk/modified polypeptide | |
|---|---|
| composition | Z-average (d · nm) |
| AS90 | 17.617 |
| AS91 | 16.803 |
| AS92 | 14.489 |
| AS93 | 12.887 |
| AS94 | 11.778 |
| AS95 | 22.01 |
| AS96 | 16.403 |
| AS97 | 23.183 |
| AS98 | 17.824 |
| AS99 | 13.607 |
| AS100 | 11.836 |

The Z-average value of each silk/modified polypeptide composition was calculated by the Zetasizer Pro. Shown here are the Z-average values of each silk composition. The abbreviation d. nm refers to the diameter in nanometers.

TABLE 11
Molecular weight (Mw) and Polydispersity (PDI)
values of silk compositions AS90-AS100.

| Low Skid silk/modified polypeptide | | |
|---|---|---|
| composition | Mw | PDI |
| AS90 | 54620 | 1.1487 |
| AS91 | 43429 | 1.2309 |
| AS92 | 35973 | 1.3634 |
| AS93 | 32689 | 1.3752 |
| AS94 | 21057 | 1.3615 |
| AS95 | 45262 | 1.1988 |
| AS96 | 36777 | 1.1995 |
| AS97 | 35375 | 1.3112 |
| AS98 | 31978 | 1.3351 |
| AS99 | 26471 | 1.4107 |
| AS100 | 22799 | 1.5438 |
| Low Skid silk | 19500 | 2.2 |

Silk/modified polypeptide compositions AS90-AS100 were analyzed by size exclusion chromatography (SEC) column with HPLC, and values of molecular weights (Mw) and Polydispersity (PDI) are indicated.

### Example 9. Mid Skid Silk/Modified Polypeptide Compositions Isolated by Size Properties

Described herein is a novel method to generate compositions of polypeptide that are derived from B. mori silkworm cocoons and comprise of natural and modified polypeptides. This novel composition is called Mid Skid silk/modified polypeptide compositions.

The novel production method involves removing sericin through several washing steps with an organic sodium carbonate salt with tightly controlled multi-stage temperature cycles and agitation as the first step in forming natural/modified polypeptide composition. Next the silk is dried to remove remaining water at controlled temperature to maintain polypeptide composition. The silk is then dissolved in high concentration of Lithium salt at 103° C. for 1 hour to achieve the compositions of Mid Skid silk. The liquid solution is then filtered and purified to remove the Lithium salt leaving only the natural/modified silk compositions in solution with pure water.

Mid Skid silk/modified polypeptide compositions comprise of populations of silk/modified polypeptides with distinctive properties. Mid Skid silk/modified polypeptide composition self-assembles at 5 mg/mL.

Mid Skid silk/modified polypeptide composition comprises of a variety of populations of silk/modified polypeptides; here it was sought to isolate distinct populations based on size, by fractionating Mid Skid silk/modified polypeptides by size exclusion chromatography. A high-resolution separation of six silk compositions was achieved—AS106, AS107, AS108, AS109, AS110, and AS111. These silk compositions differ from one another by their average size, when AS106 is the largest, and AS111 is the smallest. These silk compositions self-assemble under conditions that promote self-assembly at 5 mg/mL.

The Mid Skid silk/modified polypeptide compositions described in this invention are novel compositions of silk and modified polypeptides composed of a variety of silk polypeptide populations, generated by the exclusive treatment method of natural silk produced by B. mori. These silk compositions contain modified amino acid sequences that result from the silk processing method and scale. The tight controls over temperature, silk concentration, buffers and salt concentrations, physical agitation, and purification allow for the precise development of silk compositions with a variety of performance criteria. Isolation of these populations by charge and size reveals new characteristics, like high solubility and stability in solution over time in these populations, the purification method allows us to isolate silk/modified polypeptide compositions that display biological activities and could be used for therapeutic purposes.

Silk is a complex natural biomaterial that has the potential to be utilized in various applications such as the development of implantable medical devices, and the development of soluble polypeptide compositions of medical value. Additionally, it was demonstrated that silk peptides have anti-genotoxic effects. However, silk, in its natural form, is not soluble, and silk polypeptide compositions, without the proper processing, display poor solubility in solution and tend to self-assemble and aggregate over time. The kinetics of this self-assembly is unpredictable, and highly depends on the composition of the silk polypeptides/modified composition. ovel silk/modified polypeptide compositions were produced and specific populations were isolated within these compositions. The isolation process allows for control of the properties of the silk compositions and development of products with predictable and desired characteristics.

### Generation of Mid Skid Silk/Modified Polypeptide Compositions.

Silk is washed to remove sericin at 100° C. and 60° C. with sodium carbonate and then dried at 60° C. The silk is then dissolved in 9.3 M Lithium Bromide at 103° C. for 1 hour. This dissolution step controls not only molecular weight but also the polypeptide modifications creating the natural/modified silk compositions. The silk is then filtered to remove undissolved debris and purified using 10 kDa cutoff PES hollow fiber membranes and concentrated using the same process leaving only natural/modified silk composite in solution with pure water. Every unit ops is tightly controlled for temperature, time, concentrations, agitation, and shear.

### Isolation of Mid Skid/Modified Polypeptide Compositions

### Isolation of the AS106-AS111 Silk/Modified Polypeptide Composition Component of Mid Skid Silk/Modified Polypeptide Composition.

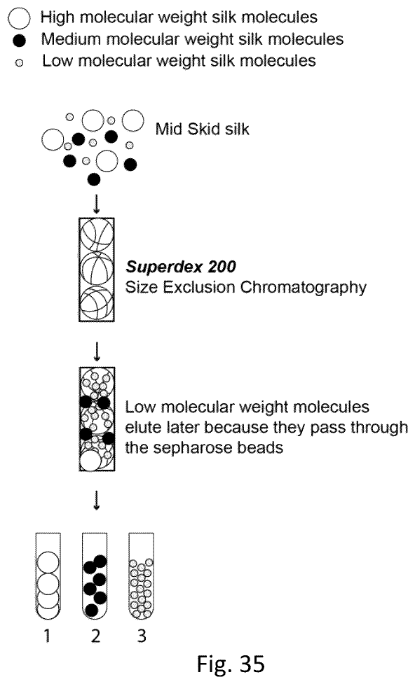

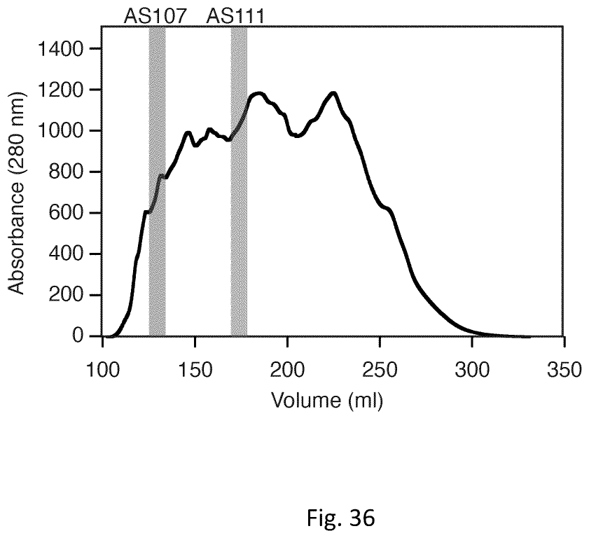

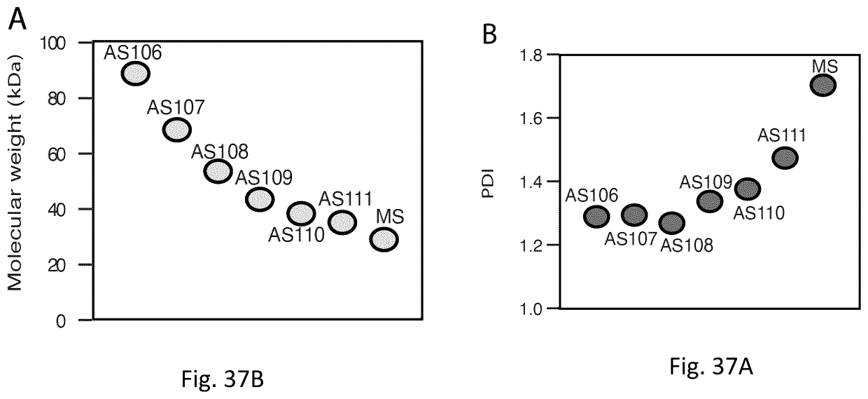

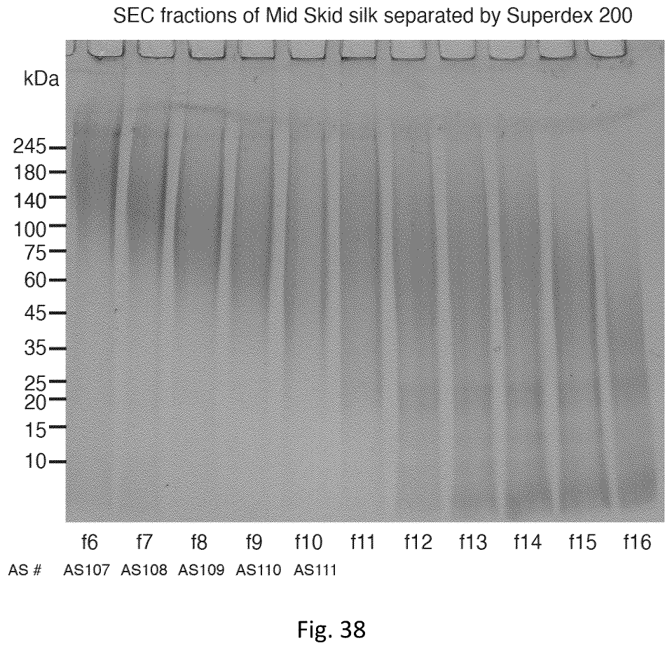

To isolate AS106-AS111, Mid Skid silk/modified polypeptide compositions were fractioned using HiLoad 26/600 Superdex 200 μg size exclusion chromatography column (FIGS. 35, 36). Tris was added to the silk preparations to a final concentration of 50 mM Tris-HCl, pH=8.0. The silk was centrifuged and filtered before loading to the HiLoad 26/600 Superdex 200 μg column, to remove any preformed aggregates. The silk compositions were fractionated by the HiLoad 26/600 Superdex 200 μg, where the largest polypeptide compositions were eluted first, and each following fraction had a population of lower molecular weight of silk compositions (FIGS. 37A-37B & FIG. 38). Mid Skid silk preparation solutions have a characteristic yellow hue, and the fractionated silk compositions had a light-yellow hue. When silk formulations AS106-AS111 are analyzed with an analytical SEC column (see materials and methods) with HPLC, each of the silk formulations demonstrates a different average molecular weight, and a different Polydispersity (PDI) value (FIGS. 37A-37B, Table 13). In general, AS106 has the highest molecular weight (89297 Da), while AS11 has the lowest molecular weight (35474 Da). Unfractionated Mid Skid silk had the lowest Mw (29265 Da), indicating that the majority of peptide population in Mid Skid silk are of lower molecular weight. The PDI values display a differential change as well. The PDI value of AS106 is relatively low (1.2866), and AS11 is higher (1.4702) (FIGS. 37A-37B, Table 13). Unfractionated Mid Skid silk has the highest PDI—1.6985, indicating a broad and diverse peptide population sizes.

### AS106-AS111 Compositions Contain Multiple Peptide Populations Sizes.

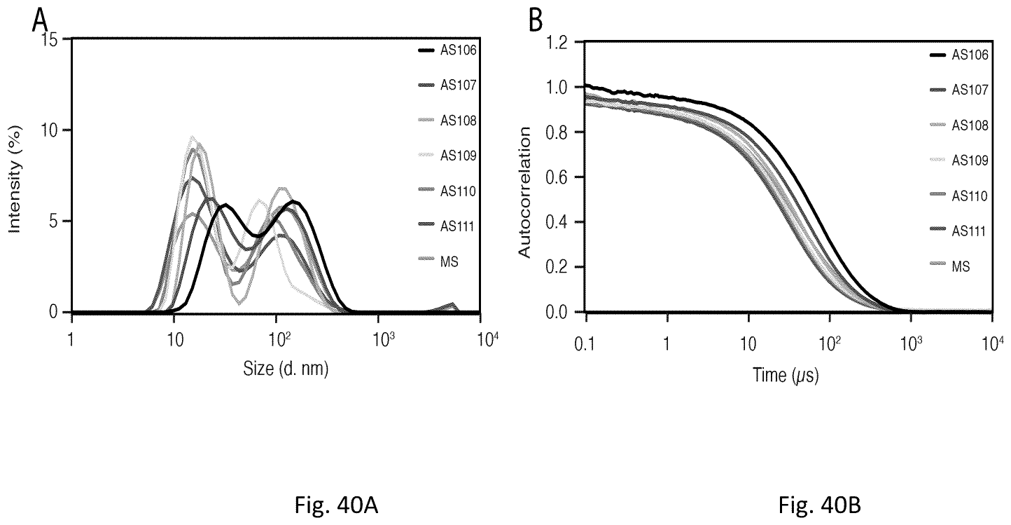

In dynamic light scattering analysis (Zetasizer Pro, FIGS. 40A-40B, Table 12), AS106-AS111 demonstrated multiple peptide size population by having two broad peaks for each fraction, similar to unfractionated Mid Skid silk (FIG. 40A). There is a shift in the molecular size of each fraction, where AS106 had the largest Z-average value (53.71 d. nm), and AS111 silk composition had the lowest (25.34 d. nm). Despite fractionation by size exclusion chromatography, each fraction contains a range of peptides in different molecular sizes, as can be observed by SDS gel electrophoresis in FIG. 38. Dynamic light scattering shows two peaks for these fractions, indicating the presence of several populations (FIG. 40A).

### Self-Assembly of Low and Mid Skid/Modified Polypeptide Compositions

Self-Assembly Assay and Data Derived from it

To study the stability of silk/modified peptide compositions in solution, Self-Assembly assays were performed at a concentration of 5 mg/mL.

The absorbance at 550 nm curves of the self-assembly assays are sigmoid and they can be described as logistic curves. The typical logistic function is:

$$f ( x ) = \frac{{A}_{max}}{1 + {e}^{- k ( t - t 0.5 )}}$$

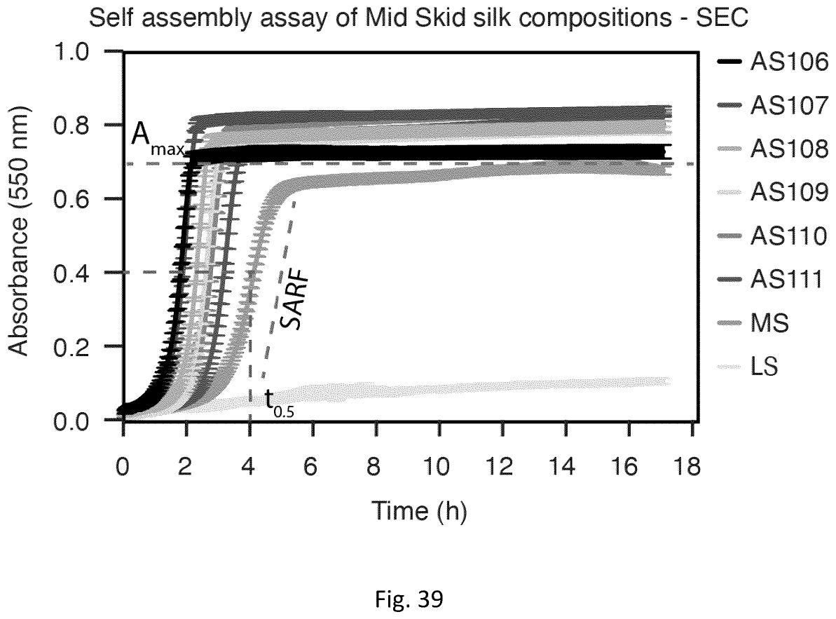

Aₘₐₓ is the maximum density of the gel formed
k is the Self-Assembly Rate Factor (SARF)
t_{0.5} is the time point at which 50% of the gel has formed
e is the exponential equation for the specific curve
(see FIG. 39 the red dotted lines for a better demonstration of how these factors from the Self-Assembly experiments were calculated)

Another parameter introduced to characterize the propensity of silk to form gels is the Self-Assembly Factor (F_{SAF}) which is:

$${F}_{SAF} = \frac{1}{t 0.5 ( min )} * {A}_{max} ( Abs ) * 1000$$

Using the experimental data from the Self-Assembly assays that were performed with the various novel isolated silk polypeptides, these parameters were calculated and used to dissect their properties (FIG. 39). Four parameters were focused on, collectively referred to as Self-Assembly kinetic factors; the Self-Assembly Rate Factor (SARF), Aₘₐₓ, t0.5, and the Self-Assembly Factor (SAF) (FIG. 39). The SARF shows how fast silk self-assembles to form gel after the reaction begins or the gelation nuclei have formed; Aₘₐₓ shows how dense is the gel that is formed after self-assembly is complete, t0.5 shows how long it takes for the self-assembly reaction to reach the point where gel density is

$$\frac{{A}_{max}}{2}$$

and SAF shows the propensity of silk to self-assemble (FIG. 39). For all parameter calculations, please see Table 14.

### AS106-AS111 Silk Compositions Demonstrate High Self-Assembly Characteristics.

Self-assembly assays revealed that Mid Skid silk/modified peptide compositions AS106-AS111 self-assemble highly efficiently under the experimental system conditions (FIG. 39). All Mid Skid fractions tested form denser gel in shorter time compared to unfractionated Mid Skid silk. This finding may indicate that components that promote self-assembly are of higher molecular weight, since the unfractionated Mid Skid silk contains a large populations of lower molecular weight peptides, but silk compositions AS106-AS111 are fractionated by size and contain higher molecular weight peptide populations (FIGS. 37A-37B & 38). Low Skid silk was used as a negative control, no self-assembly occurred after 17 hours.

### Materials and Methods Used for the Generation and Characterization of AS106-AS111.

### Size Exclusion Chromatography of Silk.

The starting material, Mid Skid silk at a concentration of 60 mg/mL, was provided by the manufacturing team. The Mid Skid silk was transferred to 50 mM Tris, pH=8.0 buffer, and centrifuged at 16000 rpm (rotor JA-18, Beckman coulter, average of 28100×g), at 4° C., for 30 min to separate formed aggregates from soluble silk. The supernatant was collected and filtered through a 0.22 μm PES filter. Then, the silk was loaded onto a HiLoad 26/600 Superdex 200 μg gel filtration column for fractionation, using the AKTA Pure 25 L system. All buffers used during fractionation were filtered through 0.22 μm PES filter as well and were degassed. The Mid Skid silk was loaded on the Superdex 200 gel filtration column, and was run with 50 mM Tris, 200 mM CaCl₂), pH8, to fractionate the Mid Skid silk. The eluted silk compositions were collected in 10 ml fractions. Fractions 5-10 (AS106, AS107, AS108, AS109, AS110, AS111) were collected. The fractions were placed in 3.5 kDa cutoff dialysis bags, and were concentrated by covering the dialysis bags with polyethylene glycol 35000 Da. Then, fractions in the dialysis bags were immersed in 160× volumes of 50 mM Tris pH=8.0 overnight, and then were immersed in a new batch of 160× volume of 50 mM Tris pH=8.0. Samples were kept at 4° C. until they were used.

### Analytical/Protein Characterization Methods.

### Protein Concentration Determination.

Protein concentration was determined by absorbance at 220 nm or 280 nm. Solubilized silk preparations were diluted until A₂₈₀ was between 0.1-1. In this range the absorbance correlates linearly with the concentration of silk in the solution and the correlation is 1AU=1 mg/mL soluble silk proteins. Final concentrations in the initial silk solution were calculated after adjustment for the dilution used for the absorbance measurement.

### Analytical Size Exclusion Chromatography.

Analytical Size Exclusion Chromatography is performed as described in detail in Example 23. Analysis was performed in a PolySep GFC P-4000 LC Column, 300 mm×7.8 mm connected to an Agilent 1260 Infinity II HPLC system with an Agilent G7162A RID Refractive Index Detector. The mobile phase used for the analysis was a solution of 0.1M NaCl, 12.5 mM Na₂HPO₄, pH 7 (the pH was adjusted with phosphoric acid and filtered through a 0.2 μm PES filter into a clean glass media bottle). 25 μL of sample were loaded on the column and the analysis was performed at 25° C. with a flow rate of 1 mL/min for 20 min. Calculation of the molecular weight of each sample was done using Agilent Technologies Open LAB CDS ChemStation Edition for LC & LC/MS Systems software Cirrus SEC data collection and molecular weight analysis software.

### SDS Polyacrylamide Gel

Mid Skid silk fractions were uploaded onto a Mini-Protean TGX precast gel, 4-20%, with a protein marker Trident Prestained Protein Ladder for molecular weight reference. The SDS polyacrylamide gel was stained using ReadyBlue™ Protein stain gel. Gels were immersed in ReadyBlue™ solution for 1 h, then destained with DI/RO water.

### Self-Assembly Assay.

The silk Self Assembly Assay (SAF) was performed in 35% v/v 2-propanol and 50 mM CH₃COONa pH=5. Each reaction was done in a final volume of 200. μL. Total silk protein concentration was 5 mg/mL. First the buffer of 50 mM CH₃COONa pH=5, 35% v/v 2-propanol was prepared. Then DI/RO water was added so that after the addition of the volume of silk protein required to reach a final concentration of 5 mg/mL the total volume would be 200 μL. The protein was added last and mixed with very gentle pipetting to reduce shearing force. The protein mixtures were placed in wells of flat-bottom 96-well plates and a layer of 100 μL of Mineral Oil carefully so as to not create any bubbles. Absorbance was recorded at 550 nm for 17h. Recorded values were exported in Excel files for storage and further analysis.

### Dynamic Light Scattering Analysis of Silk Compositions.

Mid Skid silk compositions were diluted to a concentration of 1 mg/mL and filtered with a 0.22 μm PES syringe filter. All measurements were performed with a Malvern Zetasizer Pro Red Label, detection angle of 173°. The Red Label system operates with a 10 mW He—Ne laser (633 nm). The software used is ZS XPLORER version 3.2.1.11.

All measurements were done with 4.2 mL polystyrol/polystyrene transparent cuvettes. samples were measured at 25° C., with 120 sec of equilibration time. The intensity size distributions, autocorrelation, and Z-average were measured.

TABLE 12
Z-average of AS106-AS111 calculated
by Dynamic Light Scattering.

| Mid Skid silk/modified polypeptide | Z-average (d · |
|---|---|
| composition | nm) |
| AS106 | 53.71 |
| AS107 | 39.93 |
| AS108 | 32.63 |
| AS109 | 34.87 |
| AS110 | 26.91 |
| AS111 | 25.34 |
| Mid Skid silk | 32.29 |

The Z-average value of each silk/modified polypeptide composition was calculated by the Zetasizer Pro. Shown here are the Z-average values of each silk composition. MS, Mid Skid silk.

TABLE 13
Molecular weight (Mw) and Polydispersity (PDI)
values of silk compositions AS106-AS111.

| Mid Skid silk/modified | | |
|---|---|---|
| polypeptide composition | Mw | PDI |
| AS106 | 89297 | 1.2866 |
| AS107 | 68997 | 1.2915 |
| AS108 | 53964 | 1.2662 |
| AS109 | 43762 | 1.334 |
| AS110 | 38650 | 1.3732 |
| AS111 | 35474 | 1.4702 |
| Mid Skid silk | 29265 | 1.6985 |

Silk/modified polypeptide compositions AS106, AS107, AS1108, AS109, AS110, and AS111 were analyzed by size exclusion chromatography (SEC) column with HPLC, and values of molecular weights (Mw) and Polydispersity (PDI) are indicated.

TABLE 14
Calculated self-assembly parameters
of silk compositions AS106-AS111.

| | Amax (Abs) | T_{0.5} (h) | SARF | F_{SAF} (Abs/min) |
|---|---|---|---|---|
| AS106 | 0.7226 | 1.638 | 7.599 | 7.35 |
| AS107 | 0.8258 | 1.778 | 8.270 | 7.74 |
| AS108 | 0.7803 | 2.191 | 9.959 | 5.94 |
| AS109 | 0.7678 | 2.486 | 10.66 | 5.15 |
| AS110 | 0.8159 | 2.661 | 10.38 | 5.11 |
| AS111 | 0.7844 | 3.054 | 10.47 | 4.28 |
| MS | 0.6710 | 3.843 | 7.843 | 2.91 |

For detailed description of how these parameters are calculated, please see Description of Invention section, Self-Assembly. MS, Mid Skid silk.

### Example 10: Mid Skid Silk/Modified Polypeptide Compositions Isolated by Charge and Size Properties

Described herein is a novel method to generate compositions of polypeptide that are derived from B. mori silkworm cocoons and comprise of natural and modified polypeptides. This novel composition is called Mid Skid silk/modified polypeptide compositions.

The novel production method involves removing sericin through several washing steps with an organic sodium carbonate salt with tightly controlled multi-stage temperature cycles and agitation as the first step in forming natural/modified polypeptide composition. Next the silk is dried to remove remaining water at controlled temperature to maintain polypeptide composition. The silk is then dissolved in high concentration of Lithium salt at 103° C. for 1 hour to achieve the compositions of Mid Skid silk. The liquid solution is then filtered and purified to remove the Lithium salt leaving only the natural/modified silk compositions in solution with pure water.

Mid Skid silk/modified polypeptide compositions comprise of populations of silk/modified polypeptides with distinctive properties. Mid Skid silk/modified polypeptide composition self-assembles at 5 mg/mL. Mid Skid silk/modified polypeptide composition comprises of a variety of populations of silk/modified polypeptides; here it was sought to isolate distinct populations based on charge and size, by fractionating Mid Skid silk/modified polypeptides by anion exchange chromatography and size exclusion chromatography. A high-resolution separation of five negatively-charged silk compositions was achieved—AS101, AS102, AS103, AS104, and AS105. These silk compositions differ from one another by their average size, when AS101 is the largest, and AS105 is the smallest. These silk compositions self-assemble under conditions that promote self-assembly at 5 mg/mL.

The Mid Skid silk/modified polypeptide compositions described in this invention are novel compositions of silk and modified polypeptides composed of a variety of silk polypeptide populations, generated by the exclusive treatment method of natural silk produced by B. mori. These silk compositions contain modified amino acid sequences that result from the silk processing method and scale. The tight controls over temperature, silk concentration, buffers and salt concentrations, physical agitation, and purification allow us to precisely develop silk compositions with a variety of performance criteria. Isolation of these populations by charge and size reveals new characteristics, like high solubility and stability in solution over time in these populations, the purification method allows us to isolate silk/modified polypeptide compositions that display biological activities and could be used for therapeutic purposes.

Silk is a complex natural biomaterial that has the potential to be utilized in various applications such as the development of implantable medical devices, and the development of soluble polypeptide compositions of medical value. Additionally, it was demonstrated that silk peptides have anti-genotoxic effects. However, silk, in its natural form, is not soluble, and silk polypeptide compositions, without the proper processing, display poor solubility in solution and tend to self-assemble and aggregate over time. The kinetics of this self-assembly is unpredictable, and highly depends on the composition of the silk polypeptides/modified composition. Novel silk/modified polypeptide compositions were produced and specific populations were isolated within these compositions. The isolation process allows for control of the properties of the silk compositions and development of products with predictable and desired characteristics.

### Generation of Mid Skid Silk/Modified Polypeptide Compositions.

Silk is washed to remove sericin at 100° C. and 60° C. with sodium carbonate and then dried at 60° C. The silk is then dissolved in 9.3 M Lithium Bromide at 103° C. for 1 hour. This dissolution step controls not only molecular weight but also the polypeptide modifications creating the natural/modified silk compositions. The silk is then filtered to remove undissolved debris and purified using 10 kDa cutoff PES hollow fiber membranes and concentrated using the same process leaving only natural/modified silk composite in solution with pure water. Every unit ops is tightly controlled for temperature, time, concentrations, agitation, and shear.

### Isolation of Mid Skid/Modified Polypeptide Compositions

### Isolation of the AS101-AS105 Silk/Modified Polypeptide Composition Component of Mid Skid Silk/Modified Polypeptide Composition.

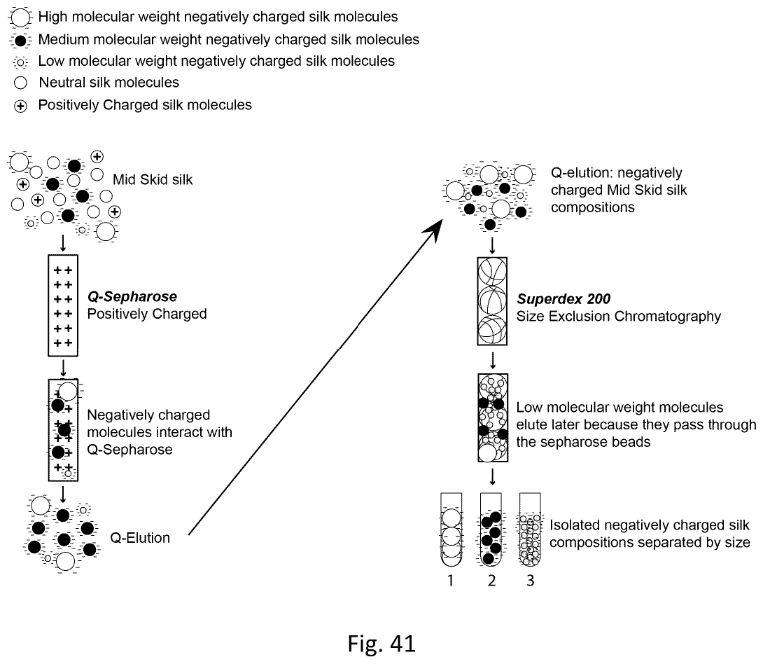

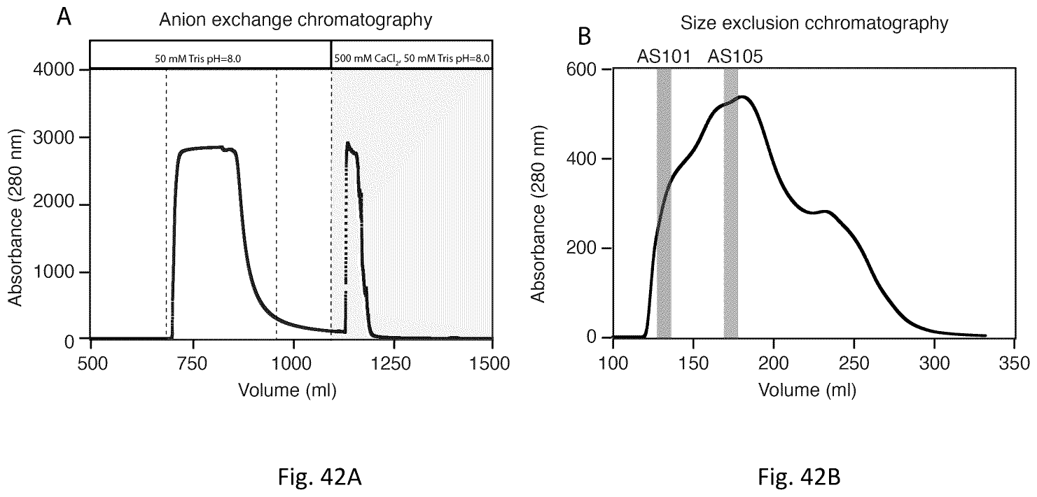

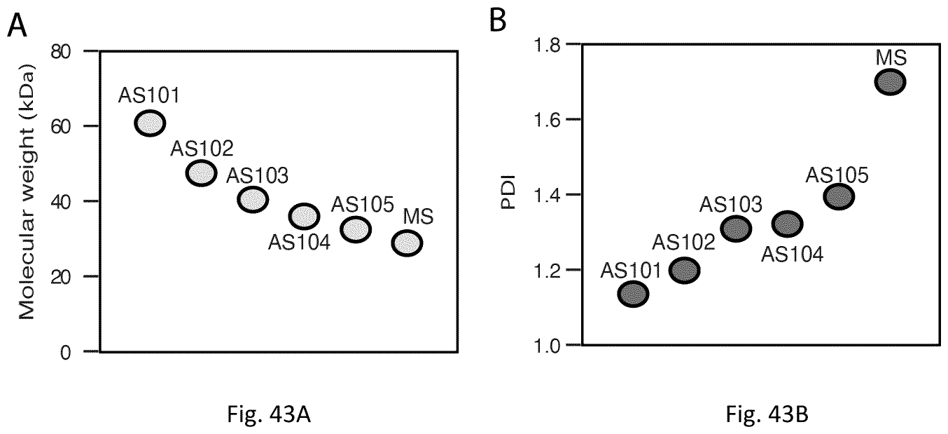

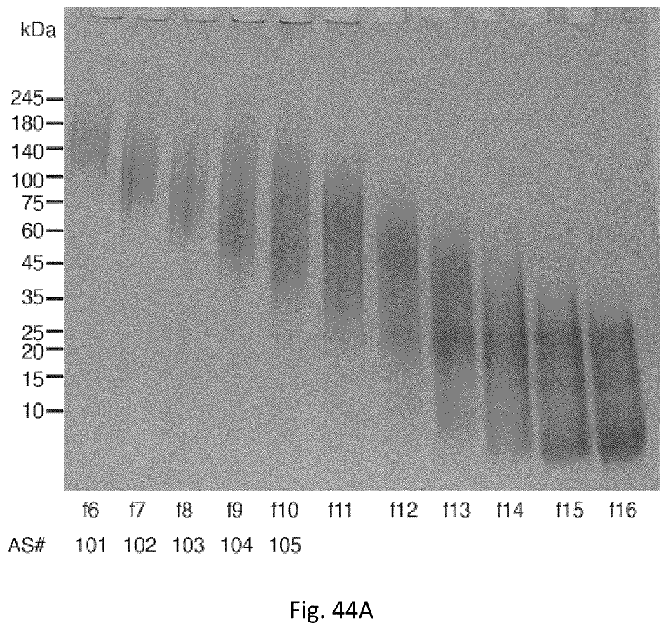

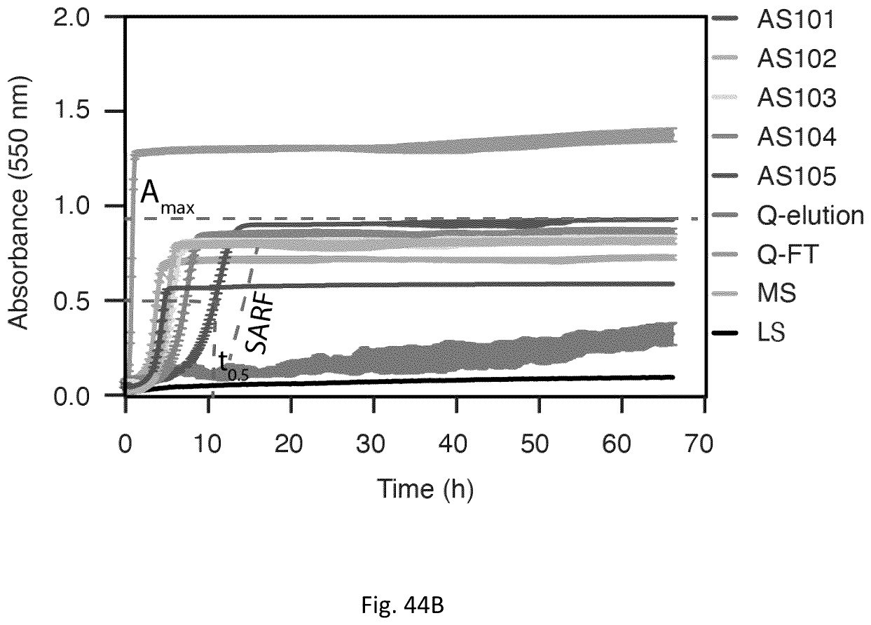

To isolate AS101-AS105, Mid Skid silk/modified polypeptide compositions were fractioned using anion exchange chromatography (Q-Sepharose chromatography), following HiLoad 26/600 Superdex 200 μg size exclusion chromatography of the Q-eluate (FIGS. 41, 42A, and 42B). Prior to chromatography, Tris was added to the silk preparations to a final concentration of 50 mM Tris-HCl, pH=8.0. The silk was centrifuged and filtered before loading to the Q-Sepharose column, to remove any preformed aggregates. The silk compositions were loaded onto the Q-Sepharose column, and the flowthrough fraction was collected. The negatively charged silk compositions were eluted using high salt buffer (50 mM Tris, 500 mM CaCl₂)). The eluted fractions were pulled together and are referred to as the Q-elution fraction. The Q-elution was further fractionated by the HiLoad 26/600 Superdex 200 μg, where the largest polypeptide compositions were eluted first, and each following fraction had a population of lower molecular weight of silk compositions (FIGS. 43A-43B & AJ). Mid Skid silk preparation solutions have a characteristic yellow hue. The Q-elution fraction has a strong yellow hue, while the flowthrough fraction is transparent, and tends to self-assemble very quickly. The Q-elution silk compositions that are fractionated by size exclusion also had a yellow hue. When silk formulations AS101-AS105 are analyzed with an analytical SEC column (see materials and methods) with HPLC, each of the silk formulations demonstrates a different average Mw, and a different Polydispersity (PDI) value (FIGS. 43A-43B, Table 16). In general, AS101 has the highest Mw (60949 Da), while AS105 has the lowest Mw (32804 Da). The PDI values display a differential change as well. The PDI value of AS101 is relatively low (1.1347), and AS105 is higher (1.3937) (FIGS. 43A-43B, Table 16). Unfractionated Mid Skid silk has an average Mw of 29265, indicating that most of the peptide population tends to have lower molecular weight than fractions AS101-AS105. The polydispersity of unfractionated Mid Skid silk is 1.6985—significantly higher than the values of fractions AS101-AS105. This indicated that the unfractionated Mid Skid silk is composed of a much diverse peptide population compared to fractions AS101-AS105, where the majority of the peptide populations have lower molecular weight.

AS101-AS105 Silk Compositions Demonstrate that Majority of the Peptide Populations are Relatively Uniform by Dynamic Light Scattering and Show Gradual Particle Size Distribution.

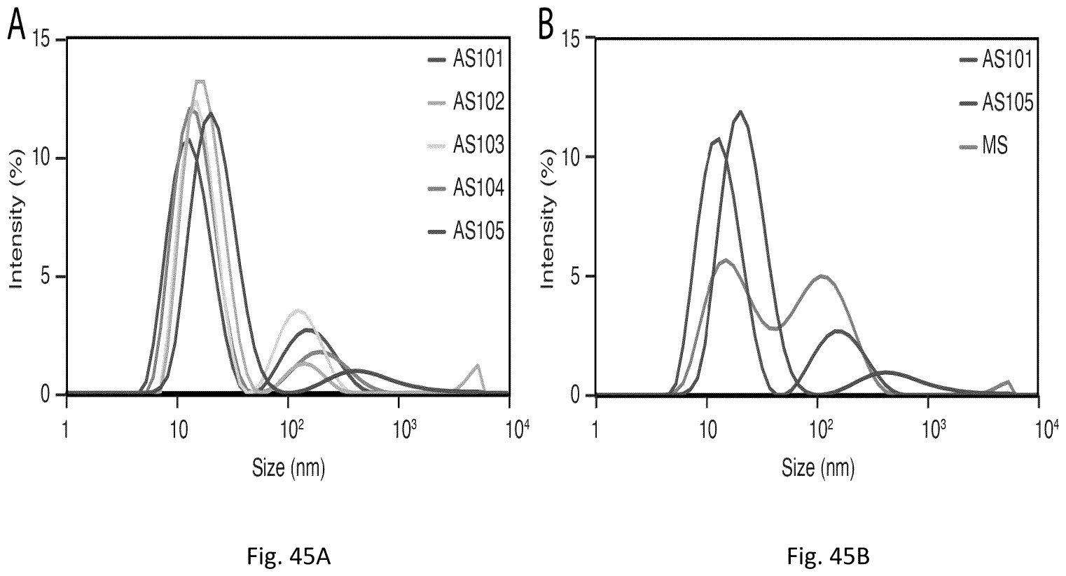

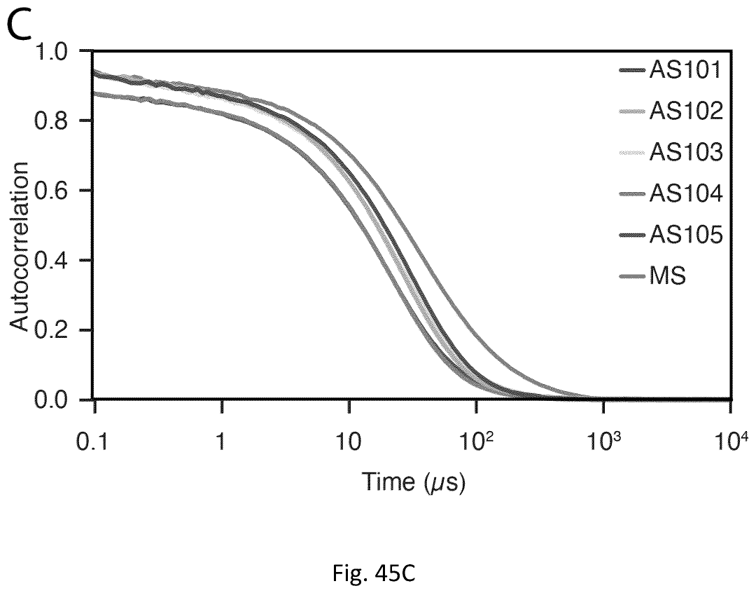

In dynamic light scattering analysis (Zetasizer Pro, FIGS. 45A-45C, Table 15), AS101-AS105 showed two peaks for each fraction, where the intensity is higher for the smaller size distribution compared to the larger size distribution peak. Comparing to unfractionated Mid Skid (FIG. 45B), which has two populations that are very close in intensity, it is evident that the Q-SEC fractionation enriched the populations that are of smaller hydrodynamic radius, and the separation between different fractions is efficient, as can be seen by the gradual decrease in size from fraction to fraction (FIGS. 45A-45B, Table 15). AS101 has the largest Z-average (21.905 d. nm), then AS102 (18.735 d. nm), and so on (Table 15), demonstrating the efficiency of the fractionation by size of the Q-elution fraction.

### Self-Assembly of Low and Mid Skid/Modified Polypeptide Compositions

Self-Assembly Assay and Data Derived from it

To study the stability of silk/modified peptide compositions in solution, Self-Assembly assays were performed at a concentration of 5 mg/mL.

The absorbance at 550 nm curves of the self-assembly assays are sigmoid and they can be described as logistic curves. The typical logistic function is:

$$f ( x ) = \frac{{A}_{max}}{1 + {e}^{- k ( t - t 0.5 )}}$$

Aₘₐₓ is the maximum density of the gel formed
k is the Self-Assembly Rate Factor (SARF)
t_{0.5} is the time point at which 50% of the gel has formed
e is the exponential equation for the specific curve
(see FIG. 44A the red dotted lines for a better demonstration of how these factors from the Self-Assembly experiments were calculated)

Another parameter introduced to characterize the propensity of silk to form gels is the Self-Assembly Factor (FSAF) which is:

$${F}_{SAF} = \frac{1}{t 0.5 ( min )} * {A}_{max} ( Abs ) * 1000$$

Using the experimental data from the Self-Assembly assays that were performed with the various novel isolated silk polypeptides, these parameters were calculated and used to dissect their properties (FIG. 44A). Four parameters were focused on, collectively referred to as Self-Assembly kinetic factors; the Self-Assembly Rate Factor (SARF), Amax, t0.5, and the Self-Assembly Factor (SAF) (FIG. 44A). The SARF shows how fast silk self-assembles to form gel after the reaction begins or the gelation nuclei have formed; Amax shows how dense is the gel that is formed after self-assembly is complete, t0.5 shows how long it takes for the self-assembly reaction to reach the point where gel density is A_max/2 and SAF shows the propensity of silk to self-assemble (FIG. 44A).

AS101-AS105 Self-Assemble in Different Kinetics to Form a Gel, while the Q-Elution Fraction Self-Assembles Poorly.

Silk compositions AS101-AS105 were tested for their ability to self-assemble and form a gel. The starting material for generating silk compositions AS101-AS105 was the Q-elution fraction (FIG. 41), which did not demonstrate significant self-assembly (FIG. 44A). However, silk compositions AS101-AS105, derived from the Q-elution fraction, self-assembled to form a gel in different kinetics (FIG. 44A, Table 17). This indicates that separating the higher-molecular-weight peptide populations from the lower-molecular-weight peptide population in Q-elution fraction allows them to self-assemble to form a gel. AS101 reached the lowest Amax, meaning the formed gel was the least dense. AS105 silk composition had the highest t0.5 value: the formation of the gel took the longest, and the gelation nuclei took longer to form.

### The Collected Q-Flowthrough Fraction Self-Assembles Exceptionally Fast and Forms the Densest Gel of all Fractions.

While fractionating silk by anion exchange, the flowthrough fraction (Q-FT) was collected (FIG. 42A) and demonstrated spontaneous slow self-assembly in 4° C. To compare the self-assembly capabilities of Q-FT to Mid Skid silk and the further fractionated silk compositions, the Q-FT in the self-assembly assay was tested. The Q-FT fraction showed extraordinary capability of self-assembly under the assay's conditions and started to self-assemble in less than 1 h (FIG. 44A, Table 17). The density of the formed gel was the highest of all silk compositions tested. These results show that the uncharged/positively charged silk compositions/modified peptides that were separated from the negatively charged population by anion exchange have a much higher tendency to self-assemble.

### Materials and Methods Used for the Generation and Characterization of AS101-AS105.

### Anion Exchange Chromatography of Silk.

Mid Skid silk was provided by the manufacturing team at a concentration of 60 mg/mL. 50 mM Tris, pH=8.0 buffer was added to the Mid Skid silk, and the silk was centrifuged at 16000 rpm (rotor JA-18, Beckman coulter, average of 28100×g), at 4° C., for 30 min to separate formed aggregates from soluble silk. The supernatant was collected and filtered through a 0.22 μm PES filter. For silk fractionation Q-Sepharose prepacked columns connected to an AKTA pure 25 L or HiPrep Q FF 16/10 20 mL Column were used. All buffers used were filtered through a 0.22 μm PES filter and degassed with sonication. Centrifuged and filtered Mid Skid silk was loaded on HiPrep Q FF 16/10 20 mL column, washed with 10 column volumes of 50 mM Tris pH=8.0, 10 column volumes of 50 mM Tris pH=8.0, 500 mM CaCl₂) and finally 10 column volumes of 50 mM Tris pH=8.0. 150 mL of centrifuged Mid Skid silk were loaded on the column with a flow rate of 5 mL/min. The flow-through was collected. The column was washed with 50 mM Tris pH=8.0 until the absorbance at 280 nm [A280] got to 100 AU. Bound protein was eluted in one step with 50 mM Tris pH=8.0, 500 mM CaCl₂) and all fractions with absorbance [A280]>500AU were pooled together. The Q-elution fraction (the eluate) was then used for further fractionation by size exclusion chromatography.

### Size Exclusion Chromatography of Silk.

The Mid Skid silk eluate fraction of the Q-Sepharose anion exchange chromatography (Q-elution) was the starting material for size exclusion chromatography. The eluate was loaded onto a HiLoad 26/600 Superdex 200 μg gel filtration column for fractionation, using the AKTA Pure 25 L system. All buffers used during fractionation were filtered through 0.22 μm PES filter as well and were degassed. The Mid Skid silk was loaded on the Superdex 200 gel filtration column, and was run with 50 mM Tris, 200 mM CaCl₂), pH=8.0, to fractionate the Q-elution Mid Skid silk. The eluted silk compositions were collected in 10 ml fractions. Fractions 6-10 (AS101, AS102, AS103, AS104, AS105) were collected, and have relatively narrow range of molecular weight. The fractions were placed in 3.5 kDa cutoff dialysis bags, and were concentrated by covering the dialysis bags with polyethylene glycol 35000 Da. Then, fractions in the dialysis bags were immersed in 160× volumes of 50 mM Tris pH=8.0 overnight, and then were immersed in a new batch of 160× volume of 50 mM Tris pH=8.0. Samples were kept at 4° C. until they were used.

### Analytical/Protein Characterization Methods.

### Protein Concentration Determination.

Protein concentration was determined by absorbance at 220 nm or 280 nm. Solubilized silk preparations were diluted until A280 was between 0.1-1. In this range the absorbance correlates linearly with the concentration of silk in the solution and the correlation is 1AU=1 mg/mL soluble silk proteins. Final concentrations in the initial silk solution were calculated after adjustment for the dilution used for the absorbance measurement.

### Analytical Size Exclusion Chromatography.

Analytical Size Exclusion Chromatography is performed as described in detail in Example 23. Analysis was performed in a PolySep GFC P-4000 LC Column, 300 mm×7.8 mm connected to an Agilent 1260 Infinity II HPLC system with an Agilent G7162A RID Refractive Index Detector. The mobile phase used for the analysis was a solution of 0.1M NaCl, 12.5 mM Na2HPO4, pH 7 (the pH was adjusted with phosphoric acid and filtered through a 0.2 μm PES filter into a clean glass media bottle). 25 μL of sample were loaded on the column and the analysis was performed at 25° C. with a flow rate of 1 mL/min for 20 min. Calculation of the molecular weight of each sample was done using Agilent Technologies Open LAB CDS ChemStation Edition for LC & LC/MS Systems software Cirrus SEC data collection and molecular weight analysis software.

### SDS Polyacrylamide Gel

Mid Skid silk fractions were loaded onto a Mini-Protean TGX precast gel, 4-20% (Bio-Rad, CAT #4561095, Batch #64518276), with a protein marker Trident Prestained Protein Ladder for molecular weight reference. The SDS polyacrylamide gel was stained using ReadyBlue™ Protein stain gel. Gels were immersed in ReadyBlue™ solution for 1 h, then destained with DI/RO water.

### Self-Assembly Assay.

The silk Self Assembly Assay (SAF) was performed in 35% v/v 2-propanol and 50 mM CH3COONa pH=5. Each reaction was done in a final volume of 200 μL. Total silk protein concentration was 5 mg/mL. First the buffer of 50 mM CH3COONa pH=5.0, 35% v/v 2-propanol was prepared. Then DI/RO water was added so that after the addition of the volume of silk protein required to reach a final concentration of 5 mg/mL the total volume would be 200 μL. The protein was added last and mixed with very gentle pipetting to reduce shearing force. The protein mixtures were placed in wells of flat-bottom 96-well plates and a layer of 100 μL of Mineral Oil carefully, so as to not create any bubbles. Absorbance was recorded at 550 nm for 24h. Recorded values were exported in Excel files for storage and further analysis.

### Dynamic Light Scattering Analysis of Silk Compositions.

Mid Skid silk compositions were diluted to a concentration of 1 mg/mL and filtered with a 0.22 μm PES syringe filter. All measurements were performed with a Malvern Zetasizer Pro Red Label, detection angle of 1. The Red Label system operates with a 10 mW He—Ne laser(633 nm). The software used isZS XPLORERversion 3.2.1.11. All measurements were done with 4.2 ml polystyrol/polystyrene transparent cuvettes. samples were measured at 25° C., with 120 sec of equilibration time. The intensity size distributions, autocorrelation, and Z-average were measured.

### Tables

TABLE 15
Z-average of AS101-AS105 calculated
by Dynamic Light Scattering.

| Mid Skid silk/modified | Z-average (d · |
|---|---|
| polypeptide composition | nm) |
| AS101 | 21.905 |
| AS102 | 18.735 |
| AS103 | 14.27 |
| AS104 | 13.07 |
| AS105 | 12.19 |
| Mid Skid silk | 30.302 |

The Z-average value of each silk/modified polypeptide composition was calculated by the Zetasizer Pro. Shown here are the Z-average values of each silk composition. The abbreviation d. nm refers to the diameter in nanometers.

TABLE 16
Molecular weight (Mw) and Polydispersity (PDI)
values of silk compositions AS101-AS105.

| Mid Skid silk/modified | | |
|---|---|---|
| polypeptide composition | Mw | PDI |
| AS101 | 60949 | 1.1347 |
| AS102 | 47772 | 1.1976 |
| AS103 | 40743 | 1.3085 |
| AS104 | 36269 | 1.3211 |
| AS105 | 32804 | 1.3937 |
| Mid Skid silk | 29265 | 1.6985 |

Silk/modified polypeptide compositions AS101, AS102, AS103, AS104, AS105, and unfractionated Mid Skid silk were analyzed by size exclusion chromatography (SEC) column with HPLC, and values of molecular weights (Mw) and Polydispersity (PDI) are indicated.

TABLE 17
Calculated self-assembly parameters of
silk compositions/modified peptides.

| | Amax (Abs) | T_{0.5} (h) | SARF | F_{SAF} (Abs/min) |
|---|---|---|---|---|
| AS101 | 0.5807 | 4.120 | 7.672 | 2.35 |
| AS102 | 0.802 | 4.932 | 9.398 | 2.71 |
| AS103 | 0.8116 | 5.416 | 7.734 | 2.50 |
| AS104 | 0.8552 | 6.866 | 7.450 | 2.08 |
| AS105 | 0.9132 | 10.27 | 7.132 | 1.48 |
| Q-FT | 1.319 | 0.9733 | 7.783 | 22.59 |
| Mid Skid silk | 0.7159 | 3.533 | 6.983 | 3.38 |

For detailed description of how these factors are calculated, please see Description of Invention section, Self-Assembly. Q-FT, flowthrough fraction collected during anion exchange chromatography using a Q column.

### Example 11. Absolute Weight Average Molecular Weight and Polydispersity of Low, Mid, and High Molecular Weight Silk by SEC-MALS

### Summary of Methodologies to Measure Molecular Weight

This example discusses the measurement of molar mass moments, specifically Mw, by SEC-MALS. Molar mass can be used interchangeably with the term “molecular weight”. For the sake of clarity, molar mass will be used in this example.

### Measuring Molar Mass Moments by SEC-RI

The methodology previously used for molar mass determination was SEC-RI. The following section is intended to describe the differences of the two methods and how both can exist in future fillings. As both methods produce a reported value of weight-averaged molecular weight (Mw), it is recommended that the Mw values be redefined as “absolute Mw” for SEC-MALS and “relative Mw” for SEC-RI

### SUMMARY

SEC is a mode of chromatographic separation and, on its own, cannot provide an absolute molar mass. Instead, SEC can provide a relative molar mass of a protein (or polymer) against a calibration curve of standard; this conventional calibration method provides a relative molar mass. SEC is often paired with a RI detector which is concentration-sensitive and molar-mass-insensitive.

Additional Information: With SEC, molecules are separated by hydrodynamic size (larger molecules having a shorter retention time than smaller molecules) which is correlated to molar mass. Herein, the retention time of the protein of interest is related to the calibration curve comprised of proteins or polymers of known molar mass. The caveat is that the unique structure or shape of a protein will impact the retention time in SEC; this may lead to variance in reported molecular weights from the use of different detectors as a specific protein of interest may differ somewhat or significantly from the calibration curve and molecules used to generate said curve. However, this method is commonly used as an industry-standard in addition to LS detectors as SEC-MALS has not been universally adopted as the singular molecular weight determination method.

### Measuring Molar Mass Moments by SEC-MALS

Here molecular weight and polydispersity as measured by Size Exclusion Chromatography-Multi-Angle Light Scattering (SEC-MALS) are reported for fractionated silk. SEC-MALS directly produces a weight averaged molar mass (Mw) measurement as compared to the relative molar mass measurement produced by a conventional calibration method such as SEC-RI.

Two detectors were used in the calculation of molar mass moments by SEC-MALS in this document: a refractive index (RI) detector and light scattering (LS) detector. RI detector directly provides the concentration of the protein. LS detector directly provides the weight-average molar mass (Mw) of the protein; the intensity of scattered light is directly proportionate to the Mw of the molecule.

### Calculation of Molar Mass Moments

The calculation of molar mass moments uses the following variables: nᵢ, Mᵢ, and cᵢ. Mᵢ, and cᵢ are directly measured by SEC-MALS. Note that the subscript “i” indicates that the value is calculated at each slice of peak of interest.
nᵢ is the number of molecules
Mᵢ is the molecular mass of the molecules (measured by LS detector)
cᵢ is the concentration of material as measured by the concentration detector (measured by RI detector)

Number-average molar mass, Mn, is related to an arithmetic mean where the total mass is divided by the number of molecules.

$${\overline{M}}_{n} = \frac{\sum {n}_{i} {M}_{i}}{\sum {n}_{i}} = \frac{\sum {c}_{i}}{\sum {c}_{i} / {M}_{i}}$$

For the weight-average molecular mass, Mw:

$${\overline{M}}_{w} = \frac{\sum {n}_{i} {M}_{i}^{2}}{{n}_{i}} = \frac{\sum {c}_{i} {M}_{i}}{\sum {c}_{i}}$$

### Calculation of Polydispersity Index (PDI)

The ratio of Mw and Mn produces the PDI value as calculated by ASTRA software. A polydisperse macromolecule has a PDI>1.05 and a monodisperse macromolecular has a PDI<1.05.

### Analytical Methods

Analysis was performed with a PolySep GFC P-4000 LC Column, 300 mm×7.8 mm with guard column connected to an Agilent 1260 Infinity II HPLC system with Wyatt LS and RI detectors (Table 18).

TABLE 18
HPLC Instrument Configuration

| Instrument Component | Vendor | Part Number |
|---|---|---|
| Sample Manager | Agilent | G5668A |
| Sample Thermostat | Agilent | G7167-60101 |
| Quaternary Pump | Agilent | G5654A |
| Column Compartment | Agilent | G7116A |
| UV Detector | Agilent | G1315C |
| LS Detector | Wyatt | miniDAWN TREOS with online |
| | | QELS |
| RI Detector | Wyatt | Optilab TrEX |

The mobile phase used for the analysis was a solution of 12.5 mM Na₂HPO₄, 100 mM NaCl titrated to a final pH of 7.0±0.2 with H₃PO₄. Before installing mobile phase on the HPLC, the solvent was filtered through a0.22 μm PES filter into a clean glass bottle. A summary of critical method parameters is described in Table 19.

TABLE 19
SEC-MALS Critical Method Parameters

| Critical Attribute | Value | |
|---|---|---|
| Target Load Mass¹ (injection volume) | 50 μg (50 μL) | |
| Flow Rate | 0.75 | mL/min |
| Column Temperature | 25° | C. |
| UV Detection Wavelength | 280 | nm |
| Run Time | 24 | min |
| Astra Collection Time | 23 | min |

¹Injection volume is varied to accommodate a 50 μg

### Data Analysis

All silk samples were processed in software ASTRA 7.3.2 following vendor recommended procedures. Reportable values of Mw, Mn, and PDI were derived from software ASTRA 7.3.2. Additional data analysis and production of figures were performed in GraphPad Prism 9 version 9.5.1 and Adobe Illustrator version 27.5.

TABLE 20
Summary of Mw and PDI for Low, Mid, and High Molecular Weight
Silk Types produced by different processes (BenchTop known
in the art and new Skid process, with different conditions)

| Production | Target Silk | Silk | | | |
|---|---|---|---|---|---|
| Scale | Conc. (%) | Type | Lot Number | Mw (kDa) | PDI |
| Benchtop | 6% | Low | EMED- | 38.7 | 2.051 |
| | | | TFF01-001- | | |
| | | | 0015 | | |
| Benchtop | 6% | Mid | TFF-001- | 51.8 | 1.445 |
| | | | 0601-M | | |
| Benchtop | 6% | High | TFF-001- | 67.6 | 1.566 |
| (60 min | | | 0603-IP | | |
| dissolution) | | | | | |
| Benchtop | 6% | High | TFF-001- | 95.1 | 1.390 |
| (15 min | | | 0602-IP | | |
| dissolution) | | | | | |
| Skid | 16.7% | Low | Skid-23164-L | 36.6 | 1.462 |
| Skid | 16.7% | Low | TFF-002- | 38.7 | 1.473 |
| | | | 23212-L | | |
| Skid | 10% | Mid | TFF-002- | 85.5 | 1.799 |
| | | | 23177-M | | |
| Skid | 10% | Mid | TFF-002- | 83.3 | 1.668 |
| | | | 23226-M | | |
| Skid | 6% | Low | TFF-003- | 35.9 | 1.416 |
| | | | 23194-L | | |
| Skid | 6% | Mid | TFF-003- | 71.4 | 1.527 |
| | | | 23201-M | | |
| Lyo Pellet | 6% | Low | LYO-004- | 41.2 | 1.575 |
| | | | 003L | | |
| Lyo Pellet | 16.7% | Low | 0075-23177- | 39.5 | 1.476 |
| | | | 001 | | |
| Lyo Pellet | 6% | Mid | LYO-004- | 64.7 | 1.661 |
| | | | 001M | | |
| Lyo Pellet | 10% | Mid | LYO-004- | 81.9 | 1.697 |
| | | | 004M | | |
| Lyo Pellet | 10% | Mid | 0092-23193- | 77.8 | 1.702 |
| | | | 001 | | |

### Example 12: Weight Average Molecular Weight and Polydispersity of Low Skid Silk/Modified Polypeptide Compositions and Mid Skid Silk/Modified Polypeptide Compositions by Size Exclusion Chromatography-Multi-Angle Light Scattering (SEC-MALS)

### Development of Mid Skid Silk/Modified Polypeptide Compositions

The development of modified polypeptide compositions of Low and Mid Skid Silk have been previously discussed as the generation of Low and Mid Skid Silk and the subsequent isolation of their modified polypeptide compositions.

### Summary of Methodologies to Measure Molecular Weight

This document discusses the measurement of molar mass moments, specifically Mw, by SEC-MALS. Molar mass can be used interchangeably with the term “molecular weight”. For the sake of clarity, molar mass will be used in this document.

### Measuring Molar Mass Moments by SEC-RI

Summary: SEC is a mode of chromatographic separation and, on its own, cannot provide an absolute molar mass. Instead, SEC can provide a relative molar mass of a protein (or polymer) against a calibration curve of standard; this conventional calibration method provides a relative molar mass. SEC is often paired with a refractive index (RI) detector which is concentration-sensitive.

Additional Information: With SEC, molecules are separated by hydrodynamic size (larger molecules having a shorter retention time than smaller molecules) which is correlated to molar mass. Herein, the retention time of the protein of interest is related to the calibration curve comprised of proteins or polymers of known molar mass. The caveat is that the unique structure or shape of a protein will impact the retention time in SEC; this may lead to variance in reported molecular weights from the use of different detectors as a specific protein of interest may differ somewhat or significantly from the calibration curve and molecules used to generate said curve. However, this method is commonly used as an industry-standard in addition to LS detectors as SEC-MALS has not been universally adopted as the singular molecular weight determination method.

### Measuring Molar Mass Moments by SEC-MALS

This document reports molecular weight and polydispersity as measured by SEC-MALS for fractionated silk. SEC-MALS produces a direct weight averaged molar mass (Mw) measurement as compared to the relative molar mass measurement produced by a conventional calibration method used by SEC-RI.

Two detectors were used in the calculation of molar mass moments by SEC-MALS in this document: a refractive index (RI) detector and light scattering (LS) detector. RI detector directly provides the concentration of the protein. LS detector directly provides the weight-average molar mass (Mw) of the protein; the intensity of scattered light is directly proportionate to the Mw of the molecule.

### Calculation of Molar Mass Moments

The calculation of molar mass moments uses the following variables: nᵢ, Mᵢ, and cᵢ. Mᵢ, and cᵢ are directly measured by SEC-MALS. Note that the subscript “i” indicates that the value is calculated at each slice of peak of interest.
nᵢ is the number of molecules
Mᵢ is the molecular mass of the molecules (measured by LS detector)
cᵢ is the concentration of material as measured by the concentration detector (measured by RI detector)

Number-average molar mass, Mn, is related to an arithmetic mean where the total mass is divided by the number of molecules.

$${\overline{M}}_{n} = \frac{\sum {n}_{i} {M}_{i}}{\sum {n}_{i}} = \frac{\sum {c}_{i}}{\sum {c}_{i} / {M}_{i}}$$

For the weight-average molecular mass, Mw:

$${\overline{M}}_{w} = \frac{\sum {n}_{i} {M}_{i}^{2}}{{n}_{i}} = \frac{\sum {c}_{i} {M}_{i}}{\sum {c}_{i}}$$

### Calculation of Polydispersity Index (PDI)

The PDI is the ratio of Mw and Mn as calculated by the program ASTRA. A macromolecule is considered to be polydisperse if the PDI>1.05.

$$PDI = \frac{{M}_{w}}{{M}_{n}}$$

### Results

### Low Skid Silk/Modified Polypeptide Compositions Isolated by Charge and Size, AS77-AS81

To isolate AS77-AS81 Low Skid silk/modified polypeptide compositions were fractioned using anion exchange chromatography (Q-Sepharose chromatography), following HiLoad 26/600 Superdex 200 μg size exclusion chromatography of the Q-eluate. Prior to chromatography, Tris was added to the silk preparations to a final concentration of 50 mM Tris-HCl, pH 8.0. The silk was centrifuged and filtered before loading to the Q-Sepharose column, to remove any pre-formed aggregates.

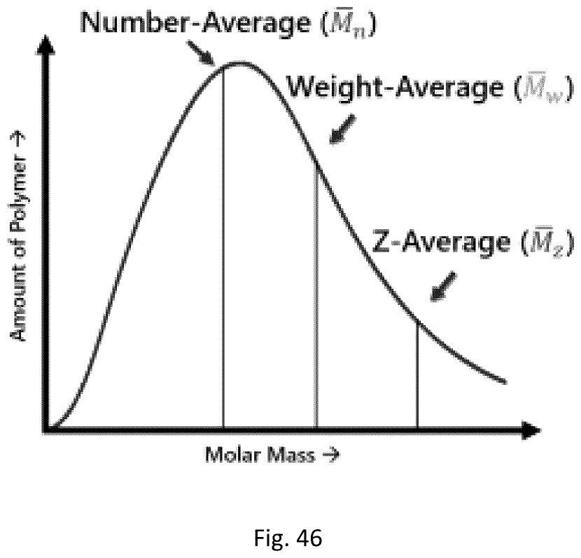

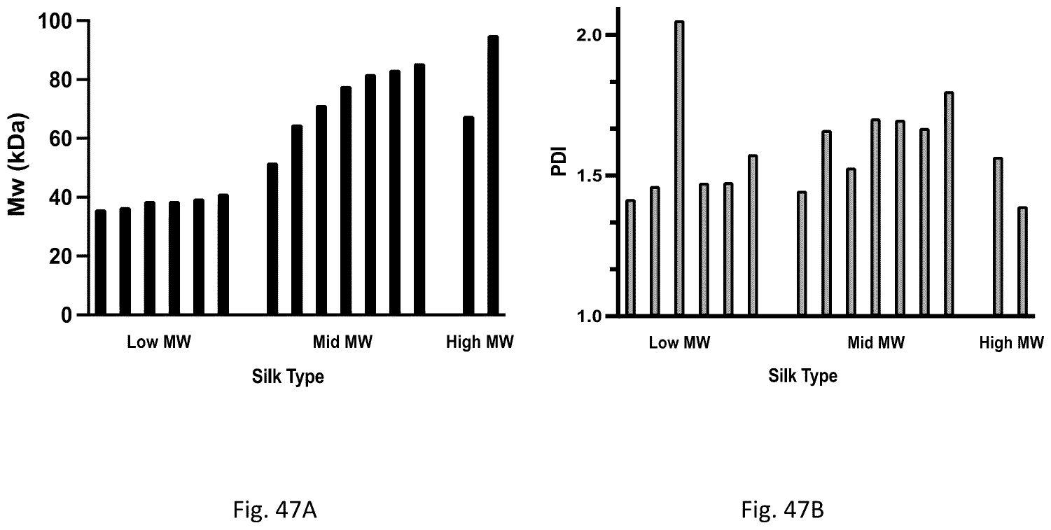

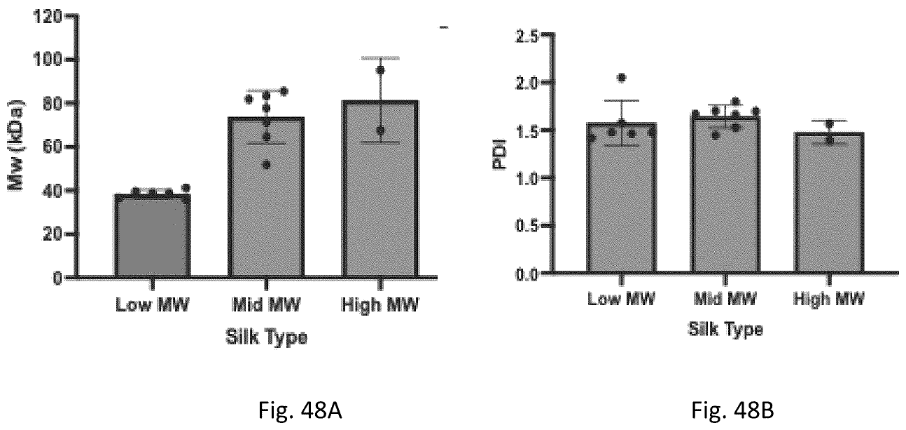

The silk compositions were loaded onto the Q-Sepharose column, and the flowthrough fraction was collected. The negatively charged silk compositions were eluted using high salt buffer (50 mM Tris, 500 mM CaCl₂)). The eluted fractions were pulled together and are referred to as the Q-elution fraction. The Q-elution was further fractionated by the HiLoad 26/600 Superdex 200 μg, where the largest polypeptide compositions were eluted first, and each following fraction had a population of lower molecular weight of silk compositions. Low Skid silk preparation solutions have a characteristic yellow hue. The Q-elution fraction has a strong yellow hue, while the flowthrough fraction is transparent, and tends to self-assemble very quickly. The Q-elution silk compositions that are fractionated by size exclusion also had a yellow hue. When silk formulations AS77-AS81 are analyzed with an analytical SEC-MALS (see materials and methods) with HPLC, the weight average molecular weight range of the 118.2 to 61.1 kDa with AS77 having the highest Mw and AS81 having the lowest Mw (FIG. 46 and Table 24). There was not a strong trend relating the PDI and fraction number but all are polydisperse (PDI>1.05). The PDI of unfractionated LS skid was significantly higher than the PDI of AS77-81 indicating the unfractionated silk has a more diverse peptide population.

Low Skid silk/modified polypeptide compositions isolated by size: AS82-AS89 To isolate AS82-AS89 Low Skid silk/modified polypeptide compositions were fractioned using HiLoad 26/600 Superdex 200 μg size exclusion chromatography column. Tris was added to the silk preparations to a final concentration of 50 mM Tris-HCl, pH 8.0. The silk was centrifuged and filtered before loading to the HiLoad 26/600 Superdex 200 μg column, to remove any pre-formed aggregates.

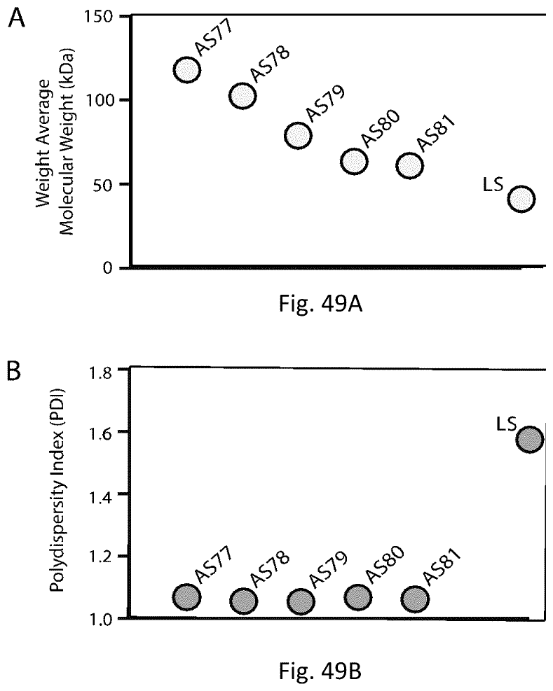

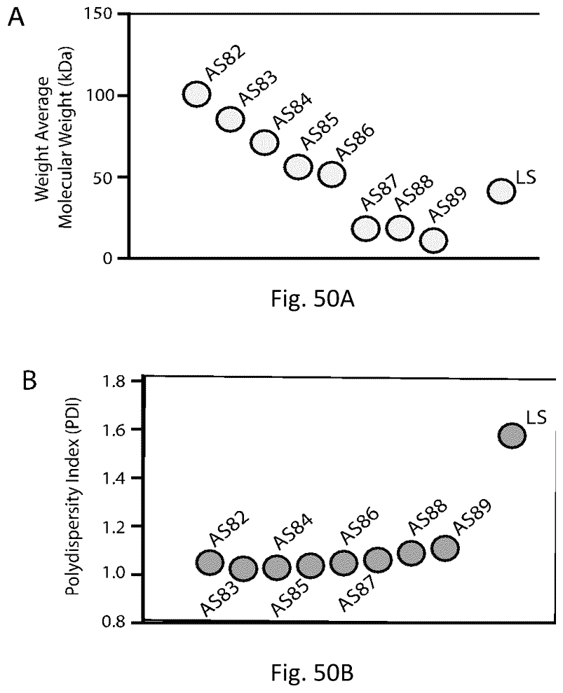

The silk compositions were fractionated by the HiLoad 26/600 Superdex 200 μg, where the largest polypeptide compositions were eluted first, and each following fraction had a population of lower molecular weight of silk compositions. When silk formulations AS82-AS89 are analyzed with an analytical SEC-MALS (see materials and methods) with HPLC, each of the silk formulations demonstrates a different average Mw and PDI values. Distinct populations were isolated by size, by fractionating Low Skid silk/modified polypeptides by preparatory scale size exclusion chromatography by FPLC; these fractions are AS82-AS89. A high-resolution separation of five silk compositions was achieved—AS82, AS83, AS84, AS85, and AS86. These silk compositions differ from one another by their average size; AS82 is the largest (100.9 kDa) and AS86 is the smallest of this set (51.5 kDa) (FIGS. 49A-49B and Table 25). Three additional fractions, AS87-AS89, were less resolved as the resolution of the Superdex 200 is not optimal for separating proteins smaller than 44 kDa. Fractions AS87, AS88, and AS89 were significantly smaller than AS82-AS86 with Mw of 18.2, 18.5 and 11.1 kDa, respectively. This higher polydispersity of AS87-AS89 is indicative of the reduced resolution and in agreement with the size measured by DLS.

### Low Skid Silk/Modified Polypeptide Compositions Isolated by Charge, Hydrophobicity, and Size: AS90-AS94 and AS95-AS100

To isolate AS90-AS100 Low Skid silk/modified polypeptide compositions were fractioned using anion exchange chromatography (Q-Sepharose chromatography), following hydrophobic interactions (HIC) chromatography, followed by HiLoad 26/600 Superdex 200 μg size exclusion chromatography of the Q-HIC-eluate (AS90-94) and of the Q-HIC-flowthrough (AS95-100).

Prior to chromatography, Tris was added to the silk preparations to a final concentration of 50 mM Tris-HCl, pH 8.0. The silk was centrifuged and filtered before loading to the Q-Sepharose column, to remove any preformed aggregates. The silk compositions were loaded onto the Q-Sepharose column, and the flowthrough fraction was collected. The negatively charged silk compositions were eluted using high salt buffer (50 mM Tris, 500 mM CaCl₂)). The eluted fractions were pulled together and are referred to as the Q-elution fraction. The Q-flowthrough fraction is colorless and tends to aggregate.

The Q-elution was further fractionated by using a Butyl ImpRes column, which separates polypeptides based on hydrophobicity. The chromatography was performed in the presence of 300 mM ammonium sulfate, to expose hydrophobic regions within the silk polypeptides. The highly charged flowthrough fraction (Q-HIC-flowthrough) was collected for further fractionation by size exclusion chromatography. The more hydrophobic, bound silk peptides were eluted using 50 mM Tris, pH 8.0 in the absence of ammonium sulfate, to reverse the exposure of the hydrophobic regions in silk polypeptides, which results in their release from the Butyl ImpRes column.

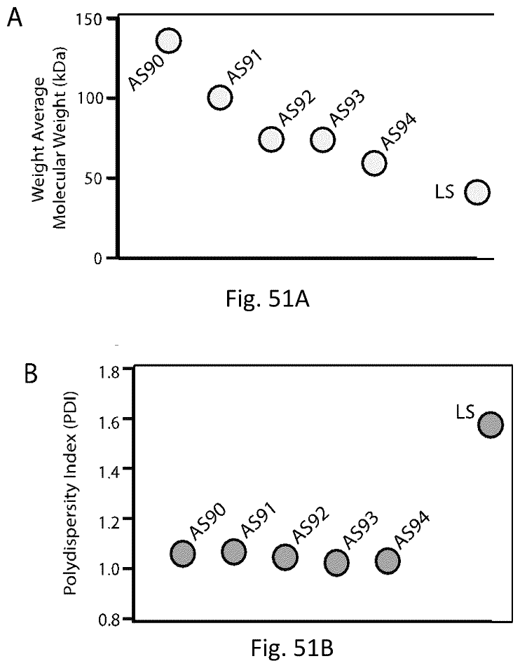

The Q-HIC-elution was further fractionated by the HiLoad 26/600 Superdex 200 μg, where the largest polypeptide compositions were eluted first, and each following fraction had a population of lower molecular weight of silk compositions (Table 25 and FIGS. 51A-51B). The fractions that were isolated are AS90-AS94. Then, the Q-HIC-flowthrough fraction was fractionated as well by the HiLoad 26/600 Superdex 200 μg, resulting in the generation of AS95-AS100.

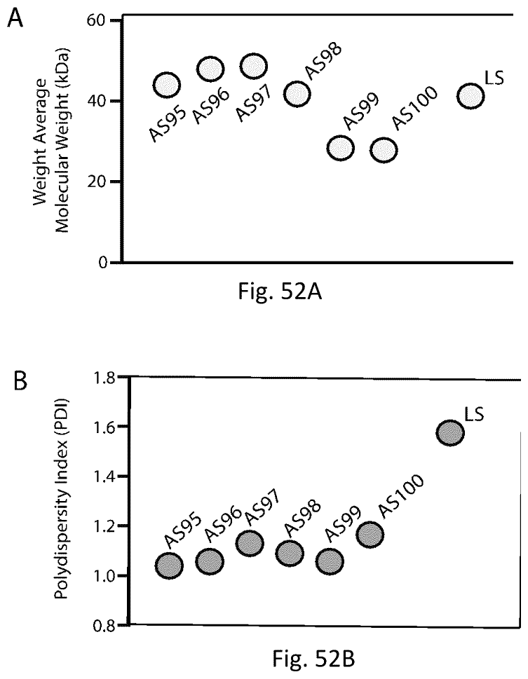

The Q-HIC Elution fraction is composed of higher-molecular-weight peptide composition, while the silk peptides that compose the Q-HIC Flowthrough fraction are eluted later, indicating smaller molecular weights (Table 26, FIGS. 51A-51B and 52A-52B). Unfractionated Low Skid silk has an average Mw of 41.2 kDa, indicating that most of the peptide population tends to have lower molecular weight than fractions AS90-AS94 (Table 26). Additionally, the PDI of unfractionated silk is significantly higher than the resultant fractions supporting that unfractionated Low Skid silk is composed of a much diverse polypeptide population compared to fractions AS90-AS100 (Table 26).

### Mid Skid Silk/Modified Polypeptide Compositions Isolated by Charge and Size: AS101-AS105

To isolate AS101-AS105, Mid Skid silk/modified polypeptide compositions were fractioned using anion exchange chromatography (Q-Sepharose chromatography), following HiLoad 26/600 Superdex 200 μg size exclusion chromatography of the Q-eluate. Prior to chromatography, Tris was added to the silk preparations to a final concentration of 50 mM Tris-HCl, pH 8.0. The silk was centrifuged and filtered before loading to the Q-Sepharose column, to remove any preformed aggregates. The silk compositions were loaded onto the Q-Sepharose column, and the flowthrough fraction was collected. The negatively charged silk compositions were eluted using high salt buffer (50 mM Tris, 500 mM CaCl₂)). The eluted fractions were pulled together and are referred to as the Q-elution fraction. The Q-elution was further fractionated by the HiLoad 26/600 Superdex 200 μg, where the largest polypeptide compositions were eluted first, and each following fraction had a population of lower molecular weight of silk compositions. Mid Skid silk preparation solutions have a characteristic yellow hue. The Q-elution fraction has a strong yellow hue, while the flowthrough fraction is transparent, and tends to self-assemble very quickly. The Q-elution silk compositions that are fractionated by size exclusion also had a yellow hue.

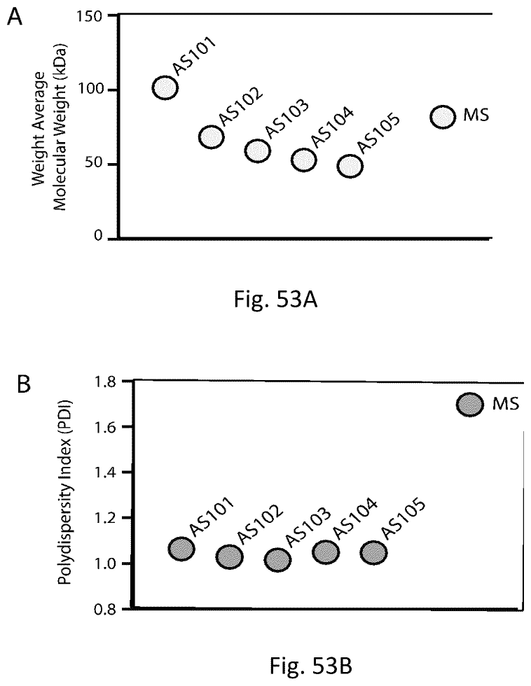

When silk formulations AS101-AS105 are analyzed with an analytical SEC-MALS (see materials and methods) with HPLC, each of the silk formulations demonstrates a unique average Mw and Polydispersity (PDI) value (FIGS. 53A-53B and Table 27). In general, AS101 has the highest Mw (101.5 kDa), while AS105 has the lowest Mw (48.9 kDa). Unlike Mw, there was no clear trend in PDI as related to fraction number. Unfractionated Mid Skid silk has an average Mw of 81.9 kDa, indicating that most of the peptide population tends to have lower molecular weight than fractions AS101-AS105. The polydispersity of unfractionated Mid Skid silk is 1.697—significantly higher than the values of fractions AS101-AS105. This indicated that the unfractionated Mid Skid silk is composed of a much diverse peptide population compared to fractions AS101-AS105, where the majority of the peptide populations have lower molecular weight.

### Mid Skid Silk/Modified Polypeptide Compositions Isolated by Size: AS106-AS111

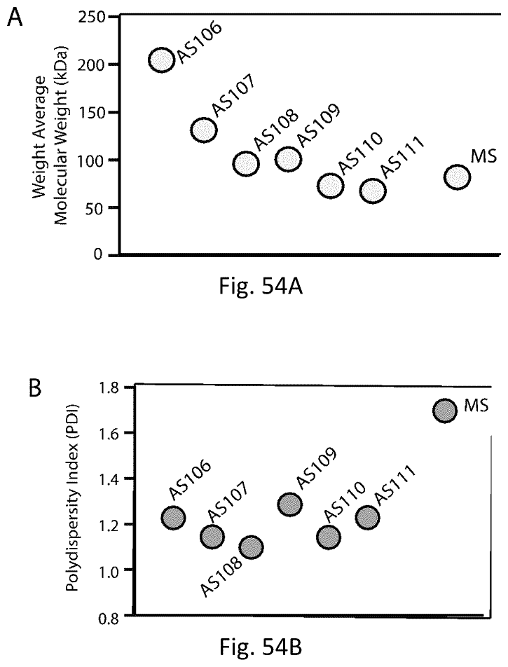

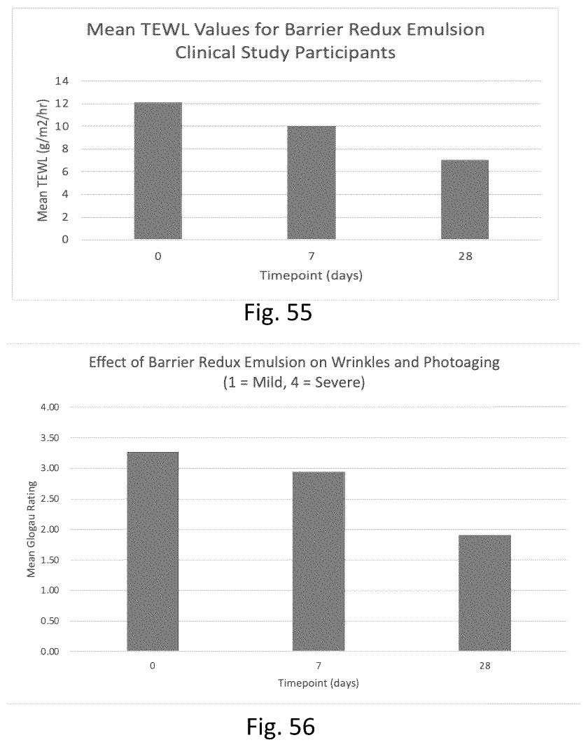

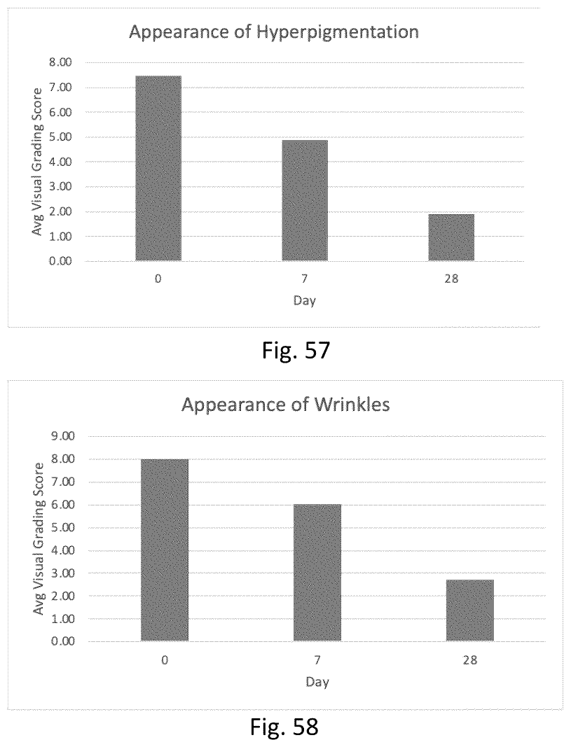

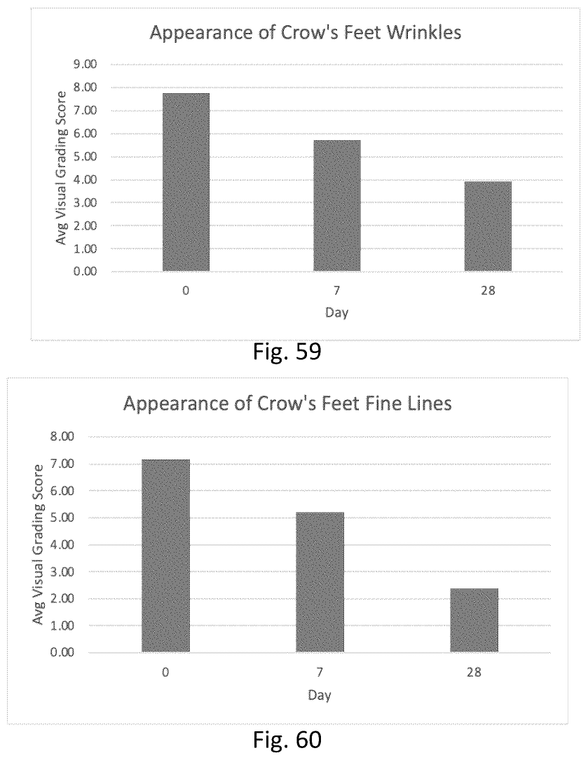

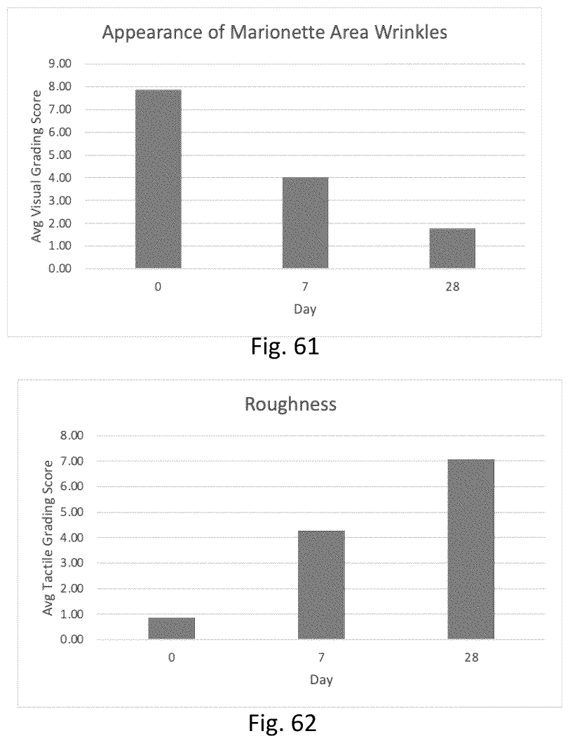

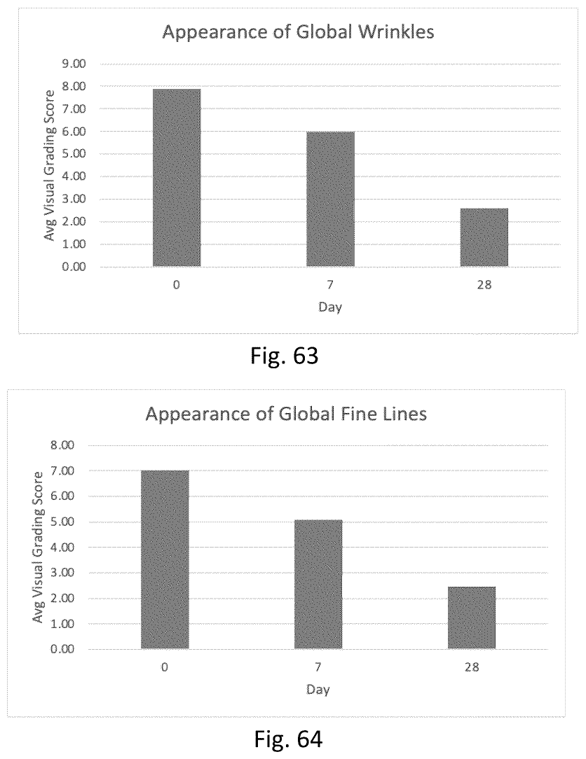

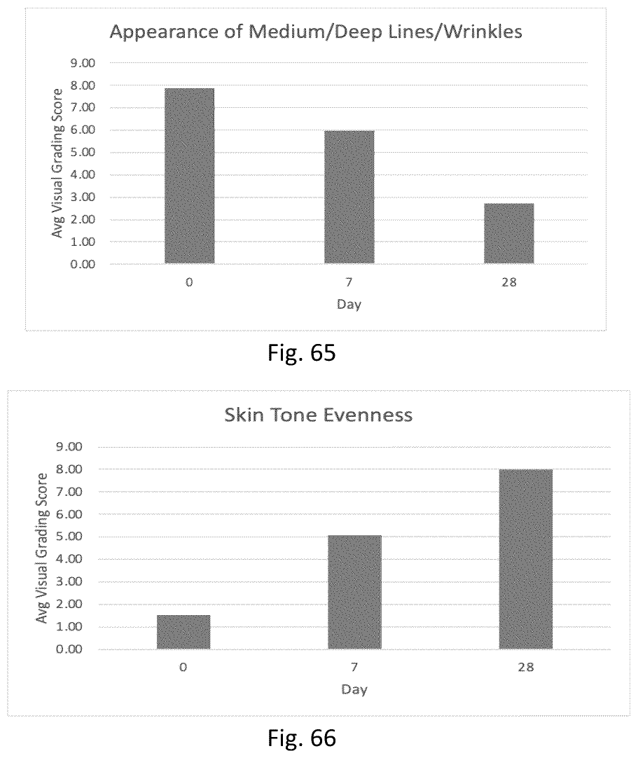

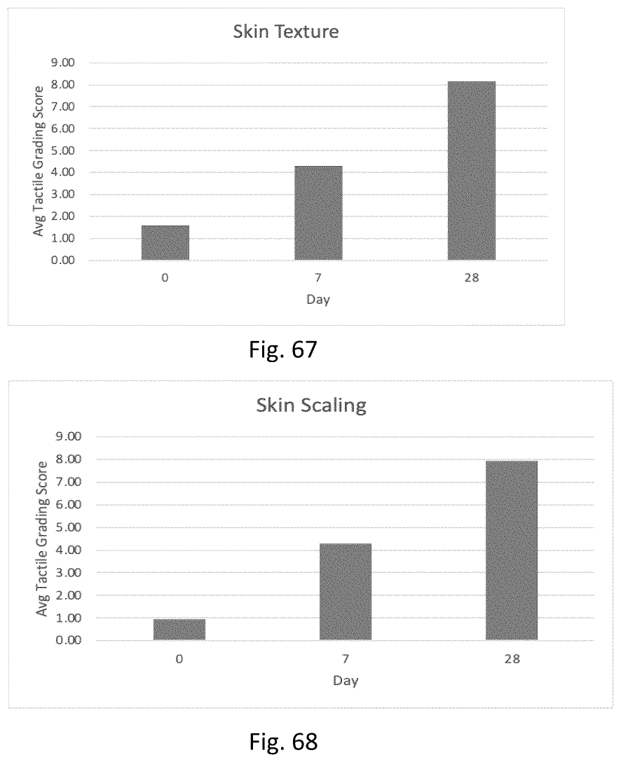

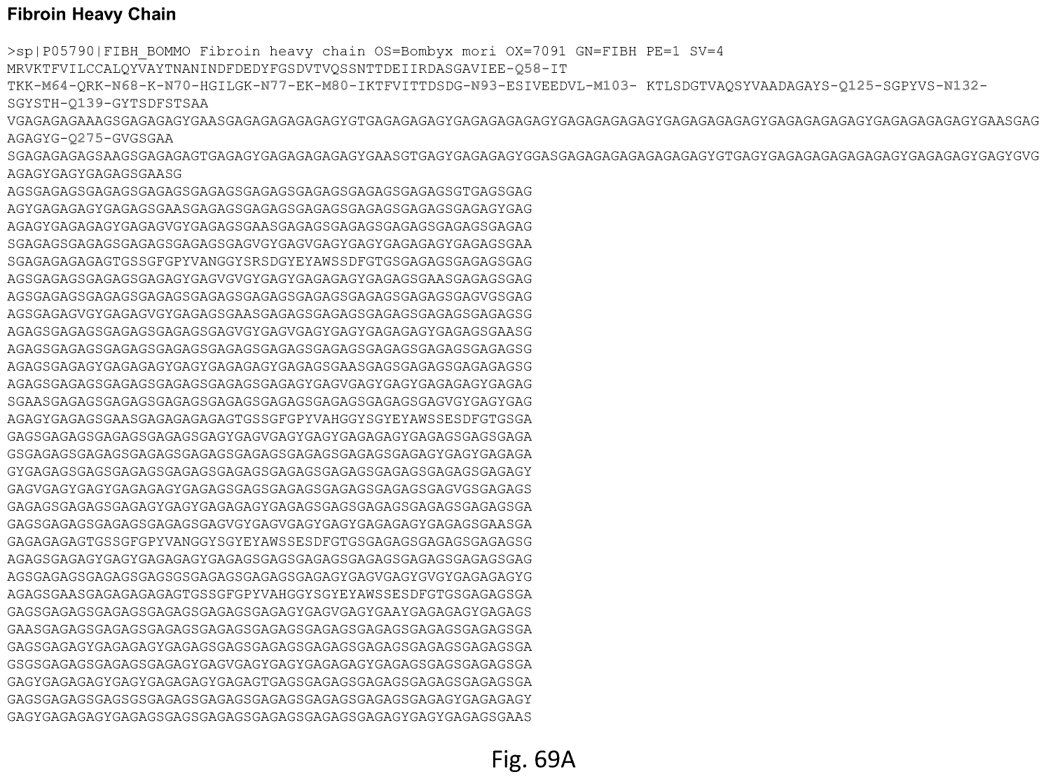

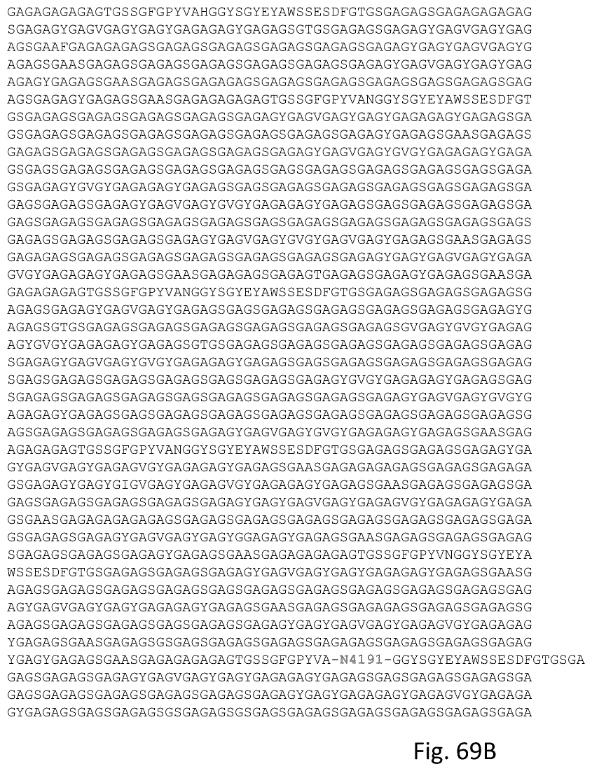

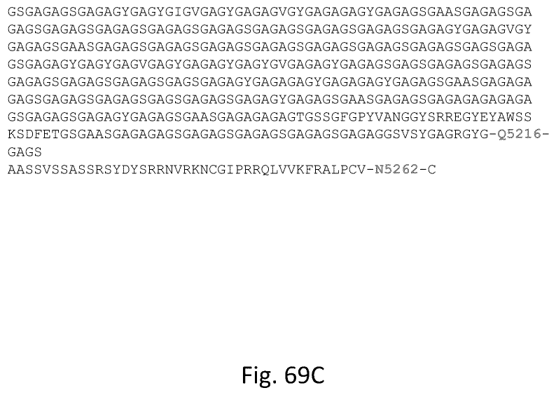

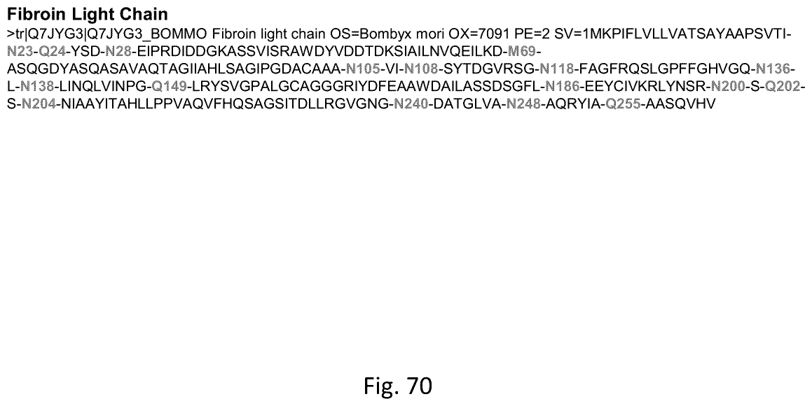

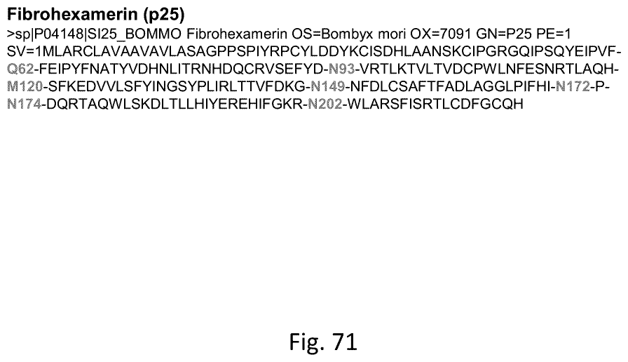

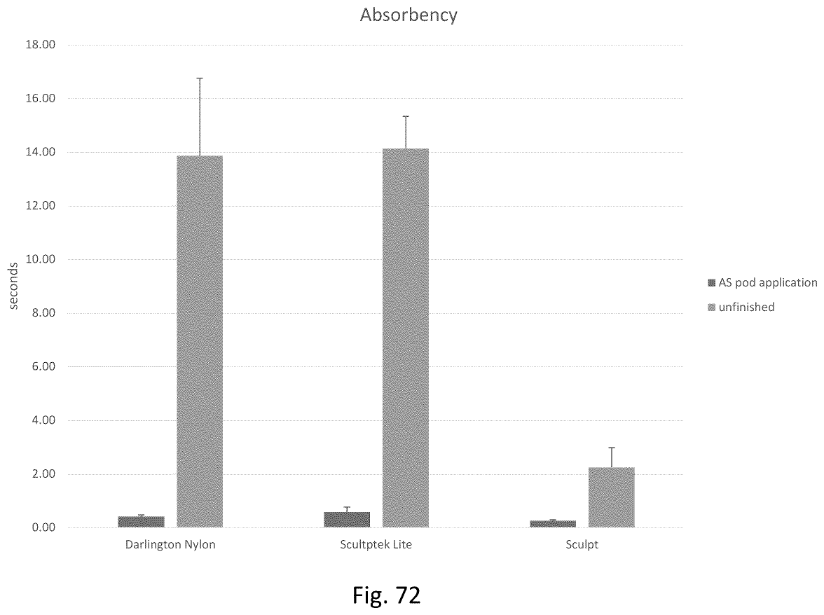

To isolate AS106-AS111 Mid Skid silk/modified polypeptide compositions was fractioned using HiLoad 26/600 Superdex 200 μg size exclusion chromatography column (FIGS. 46 & 49A-49B). Tris was added to the silk preparations to a final concentration of 50 mM Tris-HCl, pH=8.0. The silk was centrifuged and filtered before loading to the HiLoad 26/600 Superdex 200 μg column, to remove any preformed aggregates. The silk compositions were fractionated by the HiLoad 26/600 Superdex 200 μg, where the largest polypeptide compositions were eluted first, and each following fraction had a population of lower molecular weight of silk compositions (FIGS. 51A-51B & 52A-52B). Mid Skid silk preparation solutions have a characteristic yellow hue, and the fractionated silk compositions had a light-yellow hue. When silk formulations AS106-AS111 are analyzed with an analytical SEC column (see materials and methods) with HPLC, each of the silk formulations demonstrates a different average molecular weight, and a different Polydispersity (PDI) value (Table 28 and FIGS. 54A-54B). In general, AS106 has the highest molecular weight (204.4 kDa), while AS111 has the lowest molecular weight (67.4 kDa). There is not a strong trend between PDI and fraction number. Unfractionated Mid Skid silk has the highest PDI, 1.697, indicating a broad and diverse peptide population sizes.

### Analytical Methods

### Analytical SEC-MALS

Analysis was performed with a PolySep GFC P-4000 LC Column, 300 mm×7.8 mm (Phenomenex, Part No. CHO-9229) with guard column connected to an Agilent 1260 Infinity II HPLC system with Wyatt LS and RI detectors (Table 22).

TABLE 22
HPLC Instrument Configuration

| Instrument Component | Vendor | Part Number |
|---|---|---|
| Sample Manager | Agilent | G5668A |
| Sample Thermostat | Agilent | G7167-60101 |
| Quaternary Pump | Agilent | G5654A |
| Column Compartment | Agilent | G7116A |
| UV Detector | Agilent | G1315C |
| LS Detector | Wyatt | miniDAWN TREOS with online QELS |
| RI Detector | Wyatt | Optilab TrEX |

The mobile phase used for the analysis was a solution of 12.5 mM Na₂HPO₄, 100 mM NaCl titrated to a final pH of 7.0±0.2 with H₃PO₄. Before installing mobile phase on the HPLC, the solvent was filtered through a 0.22 μm PES filter into a clean glass bottle. A summary of critical method parameters is described in Table 23.

TABLE 23
SEC-MALS Critical Method Parameters

| Critical Attribute | Value | |
|---|---|---|
| Target Load Mass¹ (injection volume) | 50 μg (50 μL) | |
| Flow Rate | 0.75 | mL/min |
| Column Temperature | 25° | C. |
| UV Detection Wavelength | 280 | nm |
| Run Time | 24 | min |
| Astra Collection Time | 23 | min |

¹Samples were 1 mg/mL in 20 mM Tris pH 8 unless otherwise noted.

### Data Analysis

All silk samples were processed in software ASTRA 7.3.2 following vendor recommended procedures. Reportable values of Mw, Mn, and PDI were derived from software ASTRA 7.3.2. Additional data analysis and production of figures were performed in GraphPad Prism 9 version 9.5. land Adobe Illustrator version 27.5.

### Fractionated Low Skid Silk

Unfractionated Low Skid Silk had a Mw of 41.2 and PDI of 1.575.

### Tables

TABLE 24
Summary of Mw and PDI for fractionated
Low Skid Silk by Q-SEC (AS77-AS81)

| Fractionating | Activated Silk | | |
|---|---|---|---|
| Method | Number | Mw (kDa) | PDI |
| None | Low Skid Silk | 41.2 | 1.575 |
| Q-SEC | AS77 | 118.2 | 1.069 |
| (Q-Elution) | AS78 | 102.6 | 1.056 |
| | AS79 | 78.9 | 1.054 |
| | AS80 | 63.5 | 1.068 |
| | AS81 | 61.1 | 1.062 |

TABLE 25
Summary of Mw and PDI for fractionated
Low Skid Silk by SEC (AS82-AS89)

| Fractionating | Activated Silk | | |
|---|---|---|---|
| Method | Number | Mw (kDa) | PDI |
| None | Low Skid Silk | 41.2 | 1.575 |
| SEC | AS82 | 100.9 | 1.050 |
| | AS83 | 85.4 | 1.025 |
| | AS84 | 71.2 | 1.029 |
| | AS85 | 56.2 | 1.037 |
| | AS86 | 51.5 | 1.048 |
| | AS87 | 18.2 | 1.061 |
| | AS88 | 18.5 | 1.089 |
| | AS89 | 11.1 | 1.109 |

TABLE 26
Summary of Mw and PDI for fractionated
Low Skid Silk by Q-HIC-SEC (AS90-AS100)

| Fractionating | Activated Silk | | |
|---|---|---|---|
| Method | Number | Mw (kDa) | PDI |
| None | Low Skid Silk | 41.2 | 1.575 |
| Q-HIC-SEC | AS90 | 136.0 | 1.060 |
| (Q-Elution) | AS91 | 100.5 | 1.068 |
| | AS92 | 74.4 | 1.046 |
| | AS93 | 74.0 | 1.024 |
| | AS94 | 59.4 | 1.031 |
| Q-HIC-SEC | AS95 | 44.0 | 1.040 |
| (Q-Flow Through) | AS96 | 48.0 | 1.057 |
| | AS97 | 48.7 | 1.130 |
| | AS98 | 41.8 | 1.090 |
| | AS99 | 28.4 | 1.057 |
| | AS100 | 27.9 | 1.165 |

TABLE 27
Summary of Mw and PDI for fractionated
Mid Skid Silk by Q-SEC (AS101-AS105)

| Fractionating | Activated Silk | | |
|---|---|---|---|
| Method | Number | Mw (kDa) | PDI |
| None | Mid Skid Silk | 81.9 | 1.697 |
| Q-SEC | AS101 | 101.5 | 1.064 |
| (Q-Elution) | AS102 | 68.6 | 1.030 |
| | AS103 | 59.3 | 1.016 |
| | AS104 | 53.1 | 1.050 |
| | AS105 | 48.9 | 1.048 |

TABLE 28
Summary of Mw and PDI for fractionated
Mid Skid Silk by SEC (AS106-AS111)

| Fractionating | Activated Silk | | |
|---|---|---|---|
| Method | Number | Mw (kDa) | PDI |
| None | Mid Skid Silk | 81.9 | 1.697 |
| SEC | AS106 | 204.4 | 1.228 |
| | AS107 | 131.5 | 1.145 |
| | AS108 | 95.9 | 1.099 |
| | AS109 | 100.7 | 1.286 |
| | AS110 | 72.8 | 1.142 |
| | AS111 | 67.4 | 1.229 |

### Example 13: Barrier Redux Emulsion

The Barrier Redux Emulsion as a barrier-boosting product in the personal care industry. The product showcases Activated Silk 33B as the star ingredient. A clinical study including bioinstrumentation, expert grading and a perception questionnaire was performed to confirm the efficacy of the product, which contains 33B (an aqueous polypeptide blend having 10% solid fibroin content) at a level of 2%.

### Test Materials:

Barrier Redux Emulsion (INCI: Water, Caprylic/Capric Triglycerides, Propanediol, Populus Tremuloides Bark Extract, Soluble Fibroin, C14-22 Alcohols (and) C12-20 Alkyl Glucoside, Lysolecithin, Sclerotium Gum, Xanthan Gum, Pullulan, Allantoin, Sodium Phytate, Citric Acid)

### Study Protocol

Objective: The objective of this study was to evaluate the efficacy of a Facial Serum (Barrier Redux Emulsion) to substantiate the following the claims:
Clinically tested
Clinically proven
Reduces redness
Improves hyperpigmentation
Improves skin texture
Reduces fine lines & wrinkles

Subject Demographics: An adequate number of subjects were enrolled to ensure that a minimum of 30 completed the study. Demographics are summarized in the below table. Inclusion criteria included:
Healthy volunteers, between the ages of 21-65 years of age, with no known medical conditions that interfere with study participation.
(50% different ethnicities (non-white))
Subject has signed a written Informed Consent.
Subject has self-perceived sensitive skin.
Subject has fine lines &/or wrinkles, hyperpigmentation & poor skin texture.

TABLE 29
Subject Demographics

| SUBJECT | AGE | ETHNICITY: | SEX |
|---|---|---|---|
| 1 | 40 | WHITE | F |
| 2 | 29 | ASIAN | F |
| 3 | 47 | BLACK | M |
| 4 | 41 | WHITE | F |
| 5 | 60 | ASIAN | M |
| 6 | 58 | WHITE | F |
| 7 | 24 | BLACK | M |
| 8 | 65 | WHITE | M |
| 9 | 47 | WHITE | M |
| 10 | 52 | ASIAN | F |
| 11 | 22 | WHITE | M |
| 12 | 62 | ASIAN | F |
| 13 | 48 | BLACK | M |
| 14 | 25 | WHITE | M |
| 15 | 55 | ASIAN | F |
| 16 | 32 | ASIAN | F |
| 17 | 24 | WHITE | F |
| 13 | 61 | WHITE | F |
| 19 | 33 | black | F |
| 20 | 51 | WHITE | F |
| 21 | 43 | WHITE | F |
| 22 | 33 | ASIAN | M |
| 23 | 61 | ASIAN | M |
| 24 | 46 | WHITE | F |
| 25 | 63 | WHITE | F |
| 26 | 48 | ASIAN | F |
| 27 | 47 | WHITE | F |
| 28 | 65 | BLACK | F |
| 29 | 61 | BLACK | M |
| 30 | 43 | BLACK | F |
| 31 | 42 | WHITE | F |
| 32 | 39 | WHITE | F |
| 33 | 22 | WHITE | M |

Methods: Subjects were given the test article to use as instructed at home for 28 days. At day 7 and day 28, subjects underwent expert grading assessments, skin tactile assessments and a TEWL assessment.

### Requirements/Instructions:

TABLE 30
Requirements/Instructions

| | | |
|---|---|---|
| 1 | Alternate Names | Barrier redux emulsion |
| 2 | Manufacturing | Procedure: MFR-CAST-050 |
| | Instructions | Batch Record Form: MFR-CAST-050-F01 |
| 3 | Safety Precautions | Handle in accordance with good industrial |
| | | hygiene and safety practices. |
| 4 | Handling and Storage | Store at room temperature in an airtight |
| | | container. |
| | | Protect from light. |
| 5 | Certificate/Documents | Safety Data Sheet, Certificate of Analysis |
| 6 | Shelf Life from Date | 18 months |
| | of Manufacture | |

### Material Specifications:

Product Name: Evolved By Nature Skincare Barrier redux emulsion
Product Size: 30 mL

TABLE 31
Requirements/Instructions Material Characteristics

| Characteristic | Specification | Test Method |
|---|---|---|
| Color and | Translucent to opaque | Visual |
| Appearance | | |
| Odor | Unscented/characteristic | Smell |
| pH | 5.30-5.90 | EBN-EOP-PH-001 |
| Viscosity | 950-1390 cps | Viscometer |
| | | (Brookfield DVE) |
| | | Spindle #2, 20 rpm |
| | | 21 ± 5° C. |
| Microbial | ≤10² CFU/mL Total | USP <61> |
| enumeration | Mesophilic Microorganisms: | |
| | maximum acceptable count = | |
| | 200 | |

TABLE 32
Ingredients/Composition:

| Ingredient | Concentration (% w/w) | |
|---|---|---|
| RO/DI water | 87.4% | (±1.0%) |
| MCT Oil | 4.0% | (±0.25%) |
| Propanediol 1,3 | 3.0% | (±0.25%) |
| Activated silk (33B) | 2.0% | (±0.25%) |
| Aspen Bark | 1.5% | (±0.1%) |
| Montanov L | 1.0% | (±0.05%) |
| Ecogel | 0.5% | (±0.02%) |
| Allantoin | 0.25% | (±0.02%) |
| Sodium phytate | 0.2% | (±0.02%) |
| Citric Acid | 0.15% | (±0.1%) |

### Results

The results can be seen in Table 33. The following categories showed an improvement after 28 days:
Skin scaling, Skin Texture, Skin tone evenness
Appearance of wrinkles, deep lines, global fine lines, global wrinkles, marionette area wrinkles
Roughness, Crows feet fine lines, crows feet wrinkles
Reduction in Transepidermal water loss

TABLE 33
Results

| | | Immediately after | Day 7 (Top 2 | Day 29 (Top 2 |
|---|---|---|---|---|
| | | application (Top 2 | Responses | Responses |
| | | Responses | “Strongly | “Strongly |
| | | “Strongly Agree + | Agree + | Agree + |
| Questions | | Agree) | Agree) | Agree) |
| 1. | Skin appears healthier & younger | 51.52% | 100.00% | 100.00% |
| | looking after using the product | | | |
| 2. | Skin looks and feels smoother, | 72.73% | 100.00% | 100.00% |
| | plumper after using the product | | | |
| 3. | Skin looks and feels more hydrated | 100.00% | 100.00% | 100.00% |
| | after using the product | | | |
| 4. | Skin looks and feels more | 100.00% | 100.00% | 100.00% |
| | moisturized, and dryness reduced | | | |
| | after using this product | | | |
| 5. | Skin redness is reduced after using | 66.67% | 100.00% | 100.00% |
| | this product | | | |
| 6. | Product has a soothing and calming | 100.00% | 100.00% | 100.00% |
| | effect on skin redness and | | | |
| | irritation | | | |
| 7. | The appearance of fine lines is | 18.18% | 81.82% | 93.94% |
| | reduced after using this product | | | |
| 9. | Skin looks and feels firmer after | 27.27% | 100.00% | 100.00% |
| | using this product | | | |
| 10. | Skin looks and feels | 54.55% | 100.00% | 100.00% |
| | strengthened, more protected | | | |
| | after using this product | | | |
| 12. | Complexion appears more even | 21.21% | 78.79% | 93.94% |
| | toned | | | |
| 13. | The appearance of pores looks | 21.21% | 84.85% | 93.94% |
| | less noticeable | | | |
| 14. | The overall health of my skin is | 48.48% | 100.00% | 100.00% |
| | significantly improved after | | | |
| | using this product | | | |
| 15. | The product did not cause my | 100.00% | 100.00% | 100.00% |
| | skin to breakout | | | |
| 16. | The product feels lightweight and | 100.00% | 100.00% | 100.00% |
| | absorbs easily into skin | | | |
| 17. | The product easily layers with | 100.00% | 100.00% | 100.00% |
| | other skincare products | | | |
| 18. | This product is gentle enough to | 100.00% | 100.00% | 100.00% |
| | use every day | | | |
| 19. | 1 would purchase this product | 96.97% | 100.00% | 100.00% |
| 20. | 1 would recommend this product | 96.97% | 100.00% | 100.00% |
| | to a friend | | | |

At the conclusion of this study, Barrier Redux Emulsion validates the following claims stated in the objective:
Clinically tested
Clinically proven
Reduces redness
Improves hyperpigmentation
Improves skin texture
Reduces fine lines & wrinkles

All patents, patent applications, and published references cited herein are hereby incorporated by reference in their entirety. While the methods of the present disclosure have been described in connection with the specific embodiments thereof, it will be understood that it is capable of further modification. Further, this application is intended to cover any variations, uses, or adaptations of the methods of the present disclosure, including such departures from the present disclosure as come within known or customary practice in the art to which the methods of the present disclosure pertain.

### Example 14: Laundry Pods with Activated Silk

Three fabrics will be used for testing AS-319 moisture management properties and hand. With this study N=2 nylon is coated based and N=1 polyester based fabrics after application of Activated Silk AS-319 during the “Rinse” cycle in a front loader washing machine. The fabrics after drying will be characterized with absorbency, dry time/dry rate testing at T=0, 1, 3, 5 and 7. The hand feel will be evaluated in-house at each of the time points.

### Materials

319 lyophilized pieces made with 40% AS14A and 60% Capryl Glucoside in 2.5 gr net weight pouches.
Nylon blend fabrics:
   Sculpt
   Sculptek Lite
   Darlington

### Equipment:

Electrolux front loader washing machine model W4105H
Drying rack for fabrics

### Methods

Cut fabrics in 1 meter length.
Add N=3 pouches of AS 319 pellets to the washing machine drum
Add all the fabrics and run program Laundry Pods 25 L
The fabrics are laid on top of a drying rack

TABLE 34
Sample ID of fabrics

| Sample | | |
|---|---|---|
| ID | Reference # | AS-319 Recipe |
| 715-A-25 | Darlington Nylon | N = 3 pellets with 40% AS14A and 60% |
| 715-A-48 | Scultptek Lite | Capryl Glucoside→ mass 2.5 gr each |
| 715-A-61 | Sculpt | |
| 715-A-22 | Darlington Nylon | Unfinished |
| 715-A-58 | Scultptek Lite | |
| 715-A-70 | Sculpt | |

After the fabrics are rested overnight, they are delivered to Manufacturing Solution Center for testing. Returned fabrics after testing were screened for in-house hand assessment.

TABLE 35
Fabric Absorbency

| Sample | | AS-319 | T = 0 | | | | | | |
|---|---|---|---|---|---|---|---|---|---|
| ID | Reference # | Recipe | Ave | stdev | 1 | 2 | 3 | 4 | 5 |
| 715-A-25 | Darlington | N = 3 | 0.41 | 0.06 | 0.32 | 0.46 | 0.43 | 0.47 | 0.38 |
| | Nylon | pellets | | | | | | | |
| 715-A-48 | Scultptek | with 40% | 0.58 | 0.18 | 0.81 | 0.63 | 0.66 | 0.47 | 0.34 |
| | Lite | AS14A | | | | | | | |
| 715-A-61 | Sculpt | and 60% | 0.25 | 0.04 | 0.31 | 0.25 | 0.25 | 0.19 | 0.27 |
| | | Capryl | | | | | | | |
| | | Glucosideà | | | | | | | |
| | | mass 2.5 gr | | | | | | | |
| | | each | | | | | | | |
| 715-A-22 | Darlington | Unfinished | 13.87 | 2.89 | 14.65 | 13.63 | 9.78 | 17.84 | 13.44 |
| | Nylon | | | | | | | | |
| 715-A-58 | Scultptek | | 14.13 | 1.19 | 12.59 | 15.43 | 13.18 | 14.69 | 14.78 |
| | Lite | | | | | | | | |
| 715-A-70 | Sculpt | | 2.25 | 0.73 | 2.34 | 3.03 | 1.69 | 2.85 | 1.34 |

### Characterization

a Coated samples and unfinished fabrics are subjected to the absorbency, vertical wicking and dry rate according to AATCC test methods.
a Note, in-house environment is not humidity and temperature controlled. Results collected will only be used for internal assessment.

TABLE 36
Laundry Pod Experiment

| | front | |
|---|---|---|
| | loader | top loader |
| load (gallons) | 12.5 | 19.6 |
| load (L) | 25 | 74 |
| AS at 2.5 gr (gr/L) | 0.100 | 0.027 |
| AS at 1.25 gr (gr/L) | 0.050 | 0.017 |
| AS at 0.625 gr | 0.025 | 0.008 |
| (gr/L) | | |

TABLE 37
Pellet Composition

| | |
|---|---|
| CG (%) | 61 |
| AS (%) | 39 |

TABLE 38
Pellet Composition and Activated Silk Rate

| | | | at | AS rate |
|---|---|---|---|---|
| | | percent | application | gr/L |
| CG (gr) | 3.9 | 61% | 4.575 | 0.183 |
| AS (gr) | 2.5 | 39% | 2.925 | 0.117 |
| total (gr) | 6.4 | | 7.5 | |

TABLE 39
Wet Mass Pickup

| | | | wet pick |
|---|---|---|---|
| | dry mass | wet mass | up |
| darlington | 254.8 | 321.46 | 26% |
| Sculptek Lite | 382.84 | 587.27 | 53% |
| Sculpt | 279.31 | 448.79 | 61% |

### Example 15: Sericin Quantitation

### Quantitation of Silk Peptides in Samples

### Introduction

The following reports the findings using a LCMS method developed to quantify the relative abundance of the different silk fibroin and sericin chains.

### Sample Preparation

### Enzymatic Digestion

The samples were denatured, reduced and alkylated before 3 aliquots were taken in separate tubes for enzymatic digestion with trypsin.

TABLE 40
Percent Protein Chain

| | Cocoon | New Reactor & | New Reactor & | New Reactor & |
|---|---|---|---|---|
| Protein | Control | Process, 1^{st} Run | Process, 2^{nd} Run | Process, 3^{rd} Run |
| Chorion | 0 | 0 | 0 | 0 |
| Fibrohexamerin | 9.58 | 0.95 | 1.74 | 1.5 |
| Heavy Chain | 27.05 | 29.47 | 43.01 | 36.27 |
| Light Chain | 23.92 | 69.58 | 55.25 | 62.22 |
| Sericin | 35.27 | 0 | 0 | 0 |
| Trypsin Inhibitor | 4.19 | 0 | 0 | 0 |

### Example 16: Silk Fibroin and Surfactant Used on Synthetic Fabrics

As described in this Example, silk fibroin can be used with a surfactant on synthetic fabrics.

Activated Silk™ molecules with Capryl/Caprylyl glucoside, which is derived from sugar, provides enhanced moisture management properties on various types of nylon fabrics.

The comfort properties of clothing, particularly those leisure and sportswear, are largely determined by moisture management properties. Moisture management is the ability of a textile fabric to absorb humidity (perspiration) from skin, transport it to the outer surface and release it into the environment. With a continuous high demanding in comfort clothing, there is a great potential for growth in this field.

Moisture management performance can be enhanced by adopting microfiber technology, which increases the specific surface area of the fabric therefore allowing higher rate of liquid transportation, and by the application of various chemical finishes. The former option is quite cost intensive, making the chemical finishing option more favorable by most manufacturers.

Despite various moisture management treatment agents available for nylon, sustainable chemicals from natural derivatives with zero waste management concerns have not been widely reported. Therefore, it is of great importance to identify green chemicals for moisture management treatment that are friendly to end users, the water supply, the environment, also to the people who are doing the manufacturing.

### Coating of Nylon Fabric

Coating solutions are prepared by adjusting the pH of water to 4-4.5 by using acetic acid, then adding the Capryl/Caprylyl glucoside to a desired concentration (0.1%), and then the Activated Silk™ is added, which amount is controlled at 1:1 (v/v) ratio to Capryl/Caprylyl glucoside. The pH of the final solution is checked and adjusted if it is off the 4-4.5 range.

The coating solution is applied to nylon fabrics through a pad and cure method with the pad roller at a rate of 3 meter/minute. Wet pick up is controlled to 60% f 3% by adjusting the roller pressure.

The fabrics are dried and cured in an oven at 160° C. for 1.5 min, and then allowed to rest overnight before performance testing.

### Performance Test

Moisture management can be assessed by various test standards, such as water absorbency test, vertical wicking test, dry rate test, etc. Though results are expressed in different ways through different test methods, the characterization of moisture management performance is correlated.

In this development, modified AATCC 79—Test Method for Absorbency of Textiles is conducted to determine the moisture management performance.

### Test Results

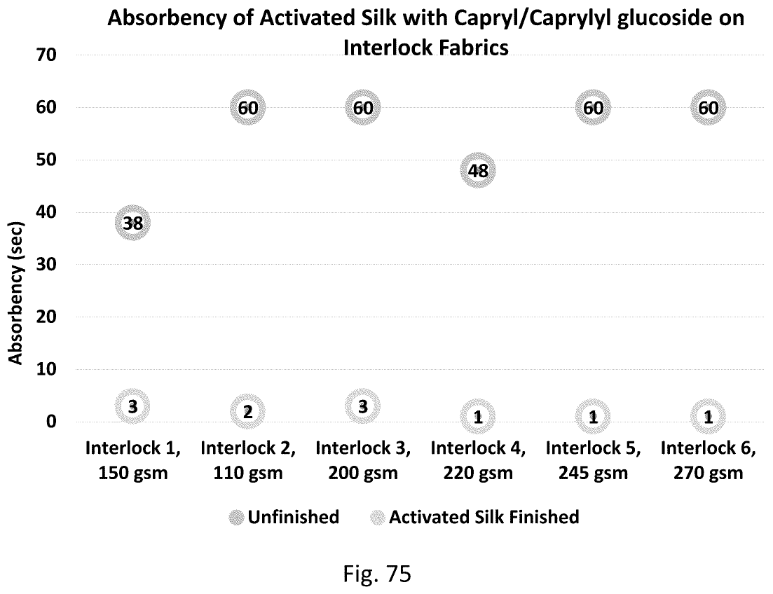

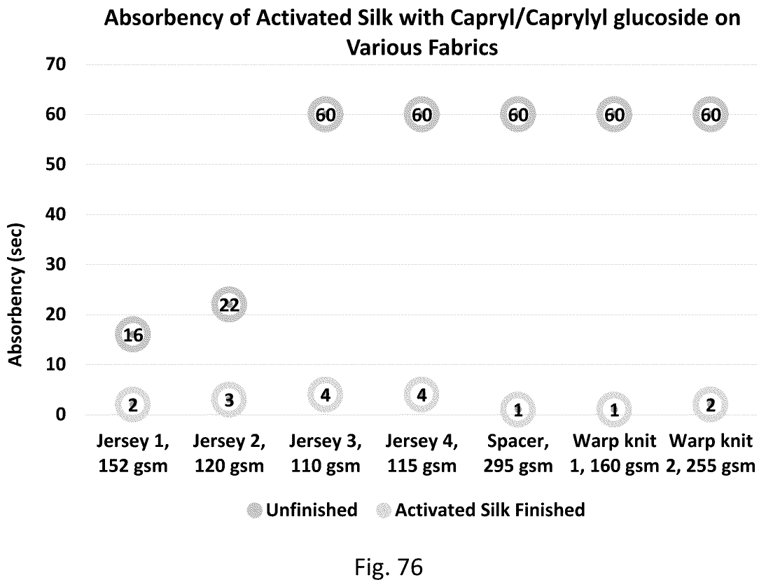

FIG. 75 and FIG. 76 are the test results of Activated Silk with Capryl/Caprylyl glucoside coating on various nylon fabrics, including interlock, jersey, warp knit and spacer structures. Results shows this novel solution has universal improvement in absorbency on various nylon fabrics, indicating its universal improvement in moisture management performance.

FIG. 75. Absorbency of Activated Silk™ with Capryl/Caprylyl glucoside coating on broad range of interlock nylon fabrics. Unfinished nylon interlock fabrics are not absorbing water having poor absorbency. After Activated Silk™ with Capryl/Caprylyl glucoside coating, the absorbency of all the nylon interlock fabrics are significantly increased.

FIG. 76. Absorbency of Activated Silk™ with Capryl/Caprylyl glucoside coating on various nylon fabrics other than interlock structure. Unfinished nylon fabrics are not absorbing water or having poor absorbency, After Activated Silk™ with Capryl/Caprylyl glucoside coating, the absorbency of all the nylon fabrics are significantly increased.

### Example 17: Silk Fibroin and Emulsifier and/or Surfactant Used on Synthetic Fabrics

As described in this Example, silk fibroin can be used with a surfactant and/or emulsifier on synthetic fabrics. In a nonlimiting example, silk fibroin fragments were used with an emulsion of three ethoxylated fatty acids to impart a smooth and drapable hand for nylon and polyester fabrics without inhibiting moisture management properties imparted by wicking agents. Wicking agents in textiles are commonly used to improve the comfort properties of leisure wear and sportswear by ameliorating the moisture management properties of nylon and polyester fabrics. However, a common issue with many wicking agents is that they are comprised of hydrophilic surfactants or polymers which attract water between the fibers of textiles causing the fabric to feel heavier and rougher than without them. With the prevalent use of these surfactants as moisture management agents, there is a potential for new developments in the field of softening agents that do not inhibit moisture management properties while also imparting a smooth and drapable hand. Currently, the majority of softening agents have two issues: (i) because many of them are oil-based their hydrophobic character inhibits water absorption and (ii) many softening agents are derived from petrochemical resources causing waste concerns and sustainability issues. Despite the relative high number of available polymeric and oil products which enhance both the hand of the fabric and moisture management capabilities, there is a limited selection of softening agents with a 100% biobased origin. Therefore, there is a need to develop 100% bio based natural softeners which impart a good hand when paired with wicking agents while not inhibiting and moisture management properties of the fabric.

Without wishing to be bound by any particular theory, the system described herein contains a mixture of ethoxylated mono- and trioleate fatty acids and hydroxy fatty acids with an average HLB value between 12-12.1 (calculated using equation 1) which acting as an emulsifier and softening agents along with activated silk acting as a moisture wicking agent. In this system, variations in the ratio of fatty acids and hydroxy fatty acids give variable HLB values (as can be seen in table 42). This in turn alters the wash fastness of the coating as well as the softening capabilities.

$$HLB = {\sum }_{i} 20 * \frac{{N}_{i}}{N} * \frac{{M}_{hi}}{{M}_{i}} \qquad Equation 1$$

Nᵢ: Moles of surfactant molecule i in solution; N: Moles of all surfactants in solution;
Mₕᵢ: Molar mass of the hydrophilic group on the surfactant molecule i; Mᵢ: Total molar mass of the surfactant molecule i.

### Coating of Nylon Fabric

An emulsifying mixture of surfactants is prepared by combining Polyoxyethylene (20) sorbitan monooleate, Polyoxyethylene (20) sorbitan trioleate, Polyoxyethylene (29) castor oil, and water in a 2:4:8:10 ratio (by mass). The mixture is then sonicated for 3 hours.

A solution of wicking agent is then prepared by adding Activated Silk™ moisture management agent to water in a concentration of 1 g/L. The solution pH is then adjusted to 4.5-5 using acetic acid.

After the wicking agent solution is made, the surfactant emulsifying mixture is then added to the solution in a mass ratio defined in Table 41.

The solution is applied to the fabric through a padding and curing method with a padding rate of 3 meters/minute and a wet pick up of 50% controlled by roller pressure

The fabrics are then cured in a drying oven at 160° C. for 1.5 to 3 minutes depending on fabric and allowed to equilibrate overnight.

### Coating of Polyester Fabric

An emulsifying mixture of surfactants is prepared by combining Polyoxyethylene (20) sorbitan monooleate, Polyoxyethylene (20) sorbitan trioleate, Polyoxyethylene (29) castor oil, and water in a 2:4:8:10 ratio (by mass). The mixture is then sonicated for 3 hours.

A solution of wicking agent is then prepared by adding Activated Silk™ moisture management agent to water in a concentration of 1 g/L. The solution pH is then adjusted to 4.5-5 using acetic acid.

After the wicking agent solution is made, the surfactant emulsifying mixture is then added to the solution in a mass ratio defined in Table 41.

The solution is applied to the fabric through a padding and curing method with a padding rate of 3 meters/minute and a wet pick up of 50% controlled by roller pressure

The fabrics are then cured in a drying oven at 135° C. and for 1-2 minutes depending on fabric and allowed to equilibrate overnight.

TABLE 41
List of ratios of silk to emulsifying mixture of surfactants (Emulsion)
based on mass and final concentration in a 1 L solution of water.

| | | | | Emulsifier | |
|---|---|---|---|---|---|
| | | Emulsifier | Silk | Mix | |
| Solution | Silk | Mix Mass | Concentration | Concentration | Silk:Emulsion |
| Sample | Mass (g) | (g) | (g/L) | (g/L) | Ratio |
| 1 | 1 | 1 | 1 | 1 | 1:1 |
| 2 | 1 | 2 | 1 | 2 | 1:2 |
| 3 | 1 | 4 | 1 | 4 | 1:4 |
| 4 | 1 | 8 | 1 | 8 | 1:8 |
| 5 | 1 | 16 | 1 | 16 | 1:16 |
| 6 | 1 | 32 | 1 | 32 | 1:32 |

TABLE 42
List of surfactants: Polyoxyethylene (20) sorbitan monooleate
(POMO), Polyoxyethylene (20) sorbitan trioleate (POTO),
Polyoxyethylene (29) castor oil (EC), in mixtures with
varying ratios and their corresponding HLB values.
Row e includes the ratio used in the emulsifying mixture
of surfactants from Table 41.

| | POMO (g) | POTO (g) | EC (g) | Water (g) | HLB |
|---|---|---|---|---|---|
| a | 2 | 4 | 0.5 | 10 | 12.56 |
| b | 2 | 4 | 1 | 10 | 12.5 |
| c | 2 | 4 | 2 | 10 | 12.39 |
| d | 2 | 4 | 4 | 10 | 12.25 |
| e | 2 | 4 | 8 | 10 | 12.09 |
| f | 2 | 4 | 16 | 10 | 11.94 |

### Testing Methods

### Fabric Performance

All samples were tested for hand feel using a panel of N=3 judges and were compared based on the factors of drapability (ease of deformation of the fabric when acted upon by the user) and smoothness (apparent roughness of fabric when rubbed by user's hands). This test follows the methods as defined by an in-house method. Inhibition of moisture management was also tested using the water absorbency test as defined by an in-house method.

### Silk Wash Fastness

Quantification of silk deposited on the surface was measured using UV/vis absorbance. Standard curves were made with varying concentrations of silk in water.

The absorbance at the 276 nm wavelength (tyrosine absorption peak) was then measured generating a linear curve correlating absorbance to solution concentration.

To test the mass of silk remaining on the fabric. The fabric was soaked in a 7.6 M LiBr solution overnight under sonication. The fabric was then removed from the solution and the UV/Vis absorbance of the solution was taken, giving a measure of the silk concentration in solution. Using the volume of solution, silk concentration in solution, and mass of the fabric it is possible to calculate the mass of silk remaining on the fabric.

### Test Results

Surfactant System with Low Weight Silk

### HLB Modulation Concentration

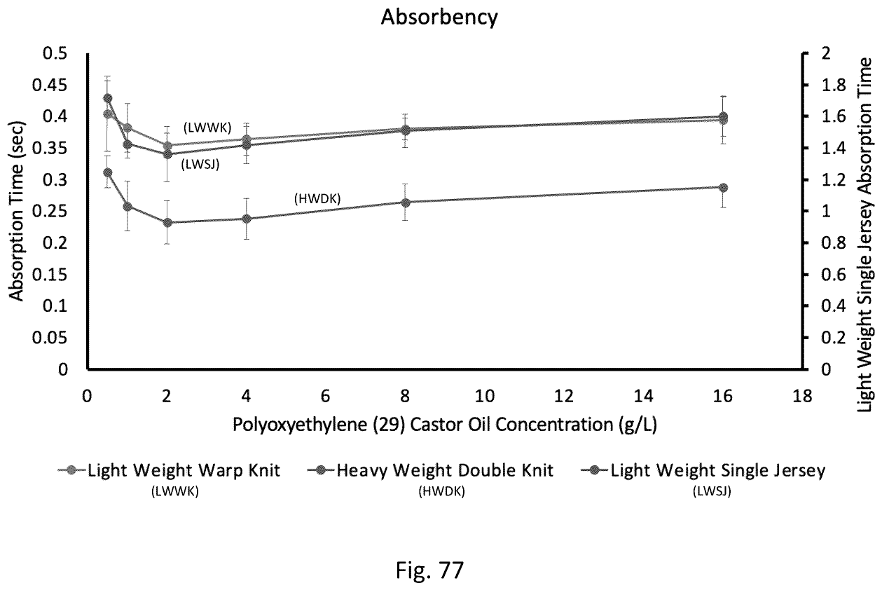

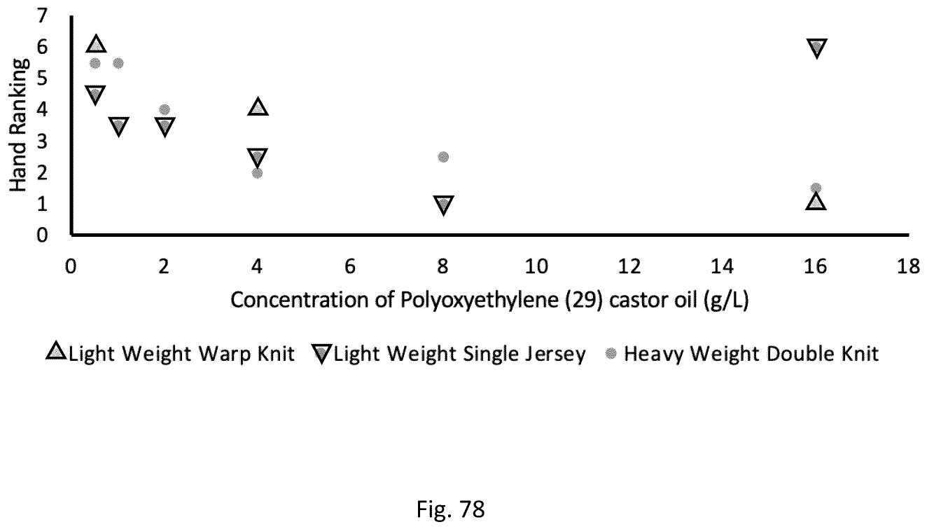

From the moisture management data, the optimal concentration for lowest absorption time is at 2 g/L Polyoxyethylene (29) castor oil (Mixture HLB: 12.39). However, as the concentration increases up to 16 g/L (Mixture HLB: 11.94) there is only an average of 17% increase in absorption time with no fabric failing the 2 second absorption time maximum criteria (FIG. 77). From a hand feel perspective, in general, as the concentration of Polyoxyethylene (29) castor oil increases the hand feel improves (FIG. 78).

FIG. 77 shows the moisture absorbency curve with no washing generated by changing the concentration of polyoxyethylene (29) castor oil in the mixture of emulsifiers (thereby changing the HLB) before adding to the coating solution. Note, in all samples the silk concentration in the coating solution 1 g/L.

FIG. 78 shows the hand feel ranking curve with no washing generated by changing the concentration of polyoxyethylene (29) castor oil in the mixture of emulsifiers (thereby changing the HLB) before adding to the coating solution. Note, in all samples the silk concentration in the coating solution 1 g/L.

### Surfactant System Concentration Study

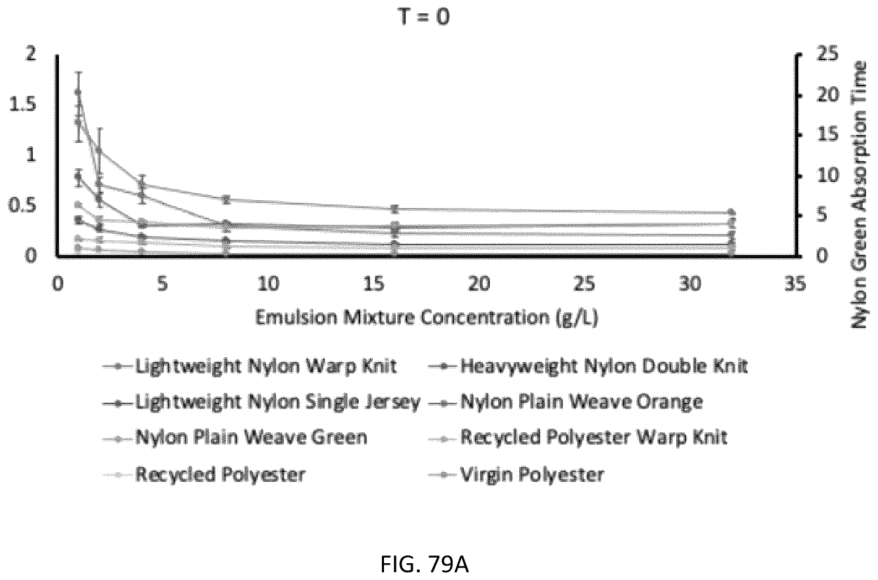

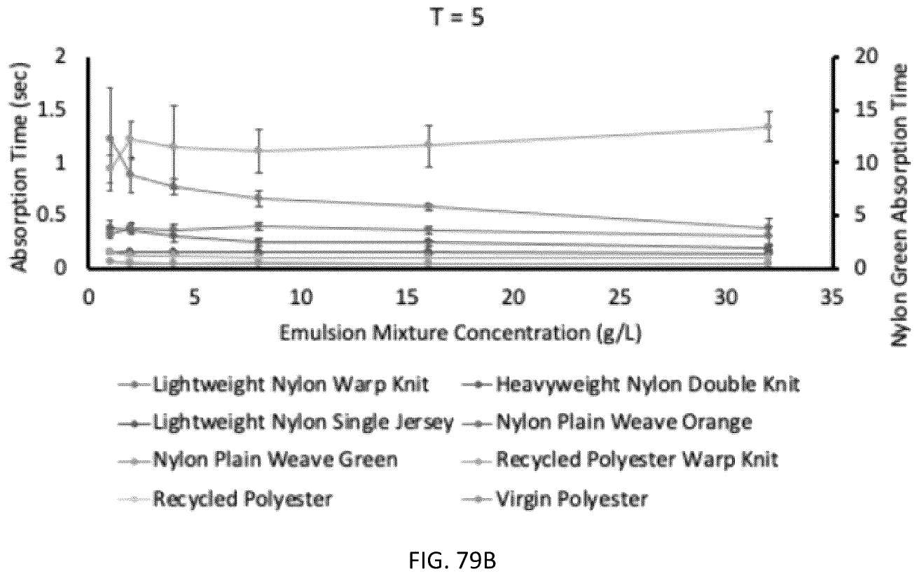

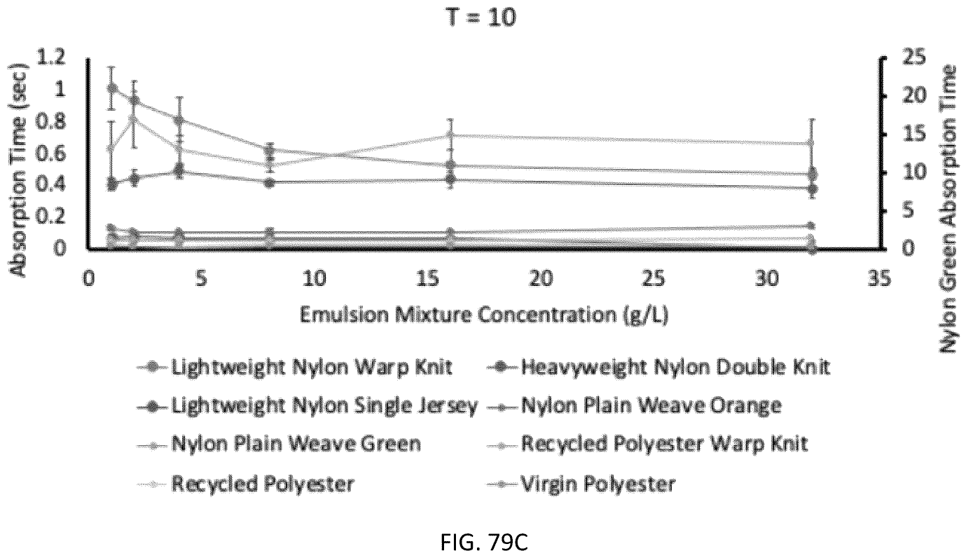

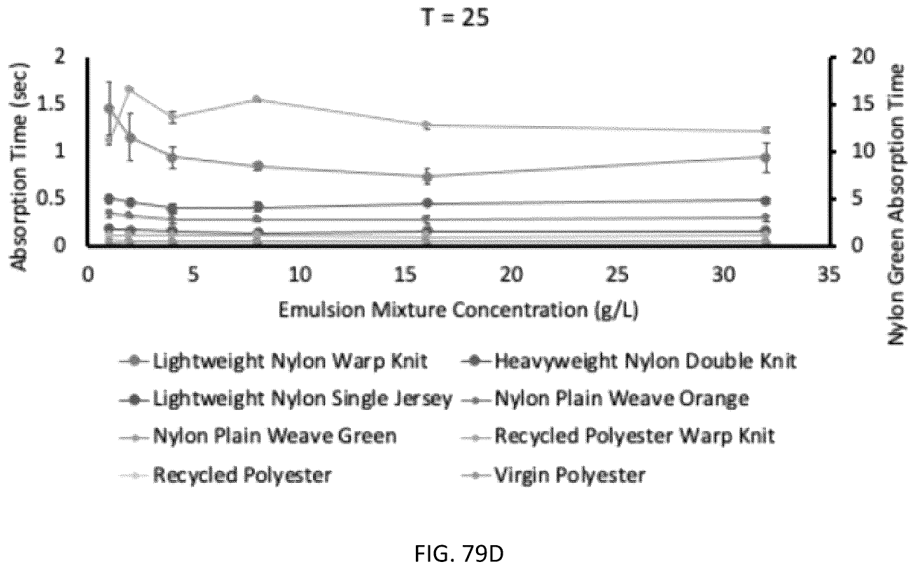

The moisture management data, as seen in FIG. 77, shows negligible degradation in moisture management performance from 0 to 25 washes on all tested polyester and nylon fabrics coated with the wicking agent and natural softener. The data shows all fabrics except for Jintex Green had absorbance times below 3 seconds from 0 to 25 washes (FIGS. 79A-79D).

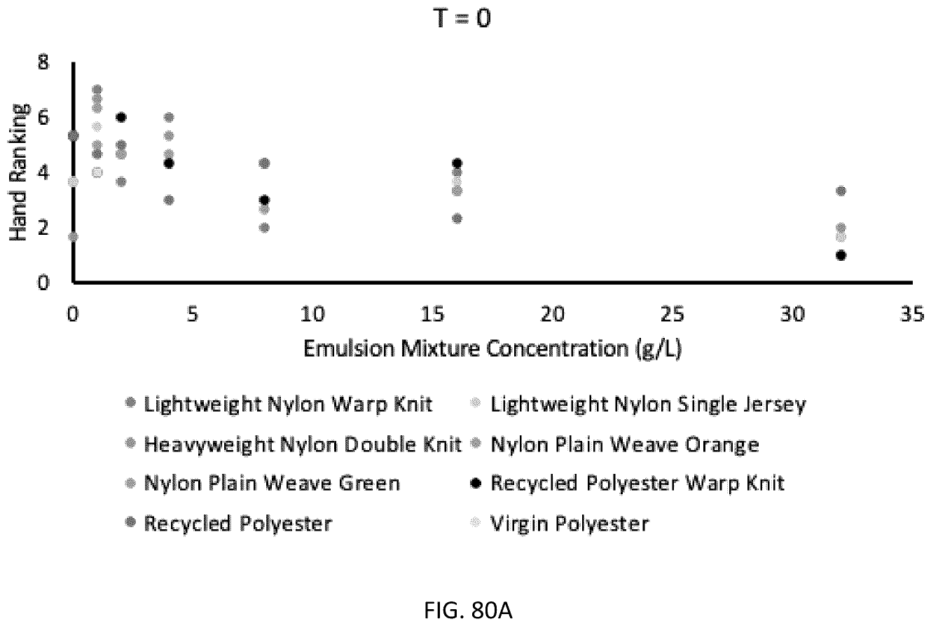

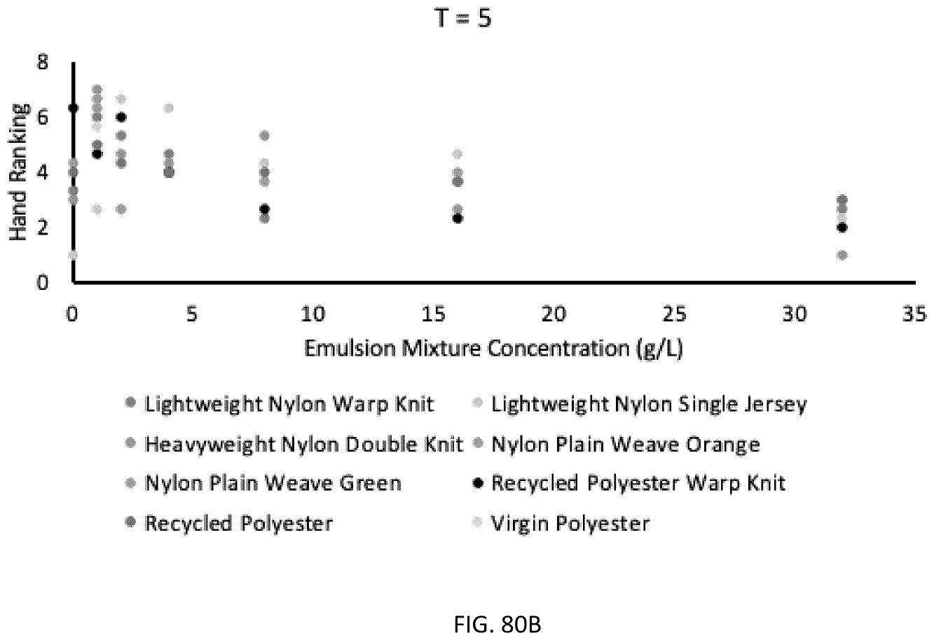

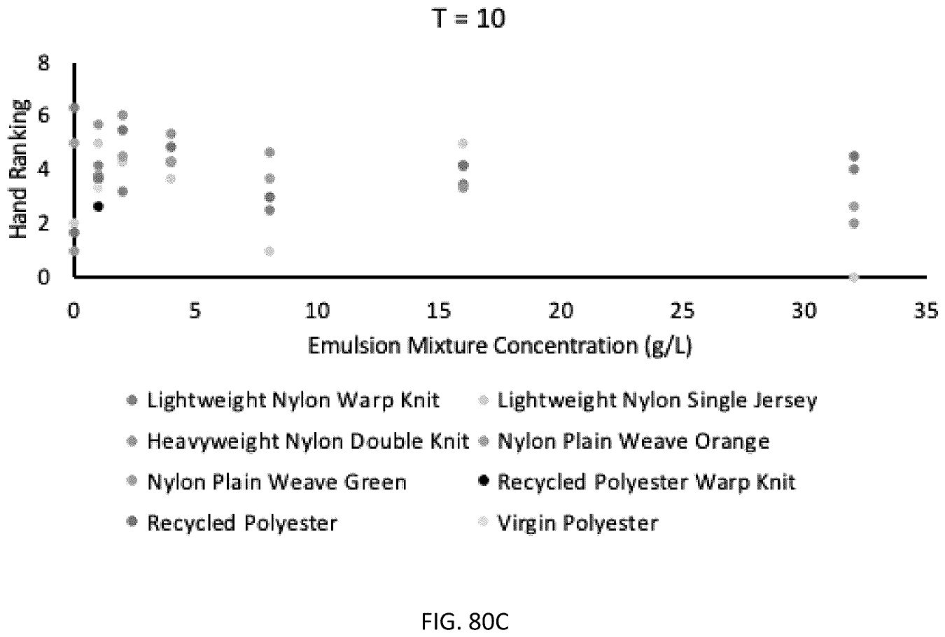

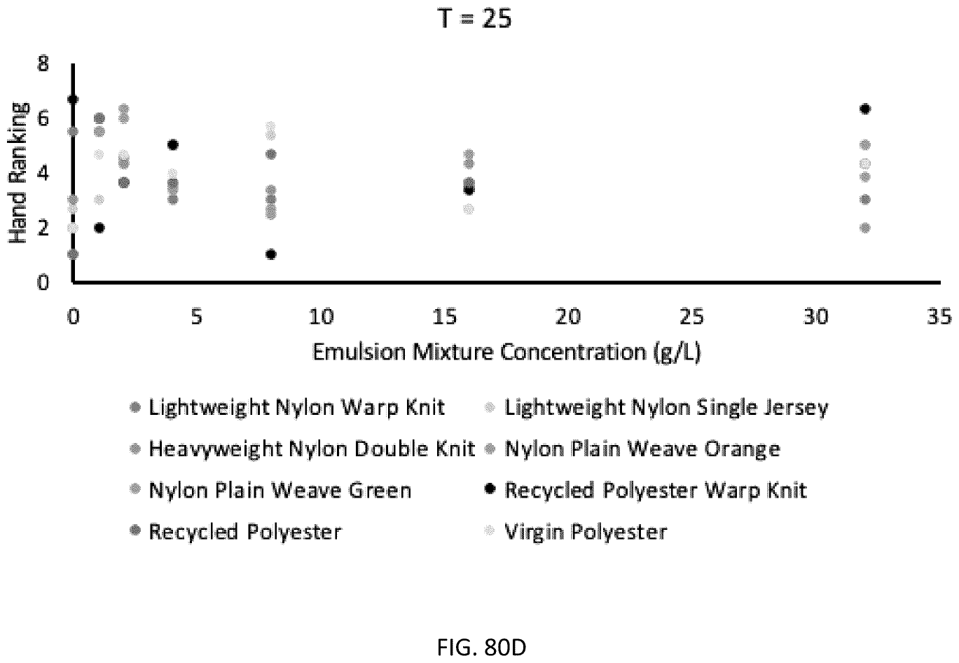

In terms of hand feel performance improvement, as seen in FIG. 78, there is a clear improvement in hand feel of fabrics as the concentration of natural softener increases at 0 washes. While there is slight degradation in performance after washing, it is still present after 5, 10, and 25 washes (FIG. 80).

FIGS. 79A-79D are charts showing the moisture management data for no washes (FIG. 79A), 5 washes (FIG. 79B), 10 washes (FIG. 79C), and 25 washes (FIG. 79D) generated by changing the concentration of the emulsion mixture (Polyoxyethylene (20) sorbitan monooleate, Polyoxyethylene (20) sorbitan trioleate, Polyoxyethylene (29) castor oil, and water in a 2:4:8:10 ratio) in the final coating solution; in all samples the silk concentration in the coating solution 1 g/L.

FIGS. 80A-80D are charts showing the hand feel ranking results from no washes (FIG. 80A), 5 washes (FIG. 80B), 10 washes (FIG. 80C), and 25 washes (FIG. 80D) generated by changing the concentration of the emulsion mixture (Polyoxyethylene (20) sorbitan monooleate, Polyoxyethylene (20) sorbitan trioleate, Polyoxyethylene (29) castor oil, and water in a 2:4:8:10 ratio) in the final coating solution; 1 is the best score and 8 is the worst hand ranking score; in all samples the silk concentration in the solution 1 g/L.

Polyoxyethylene (20) Sorbitan Monooleate with Mid Weight Silk

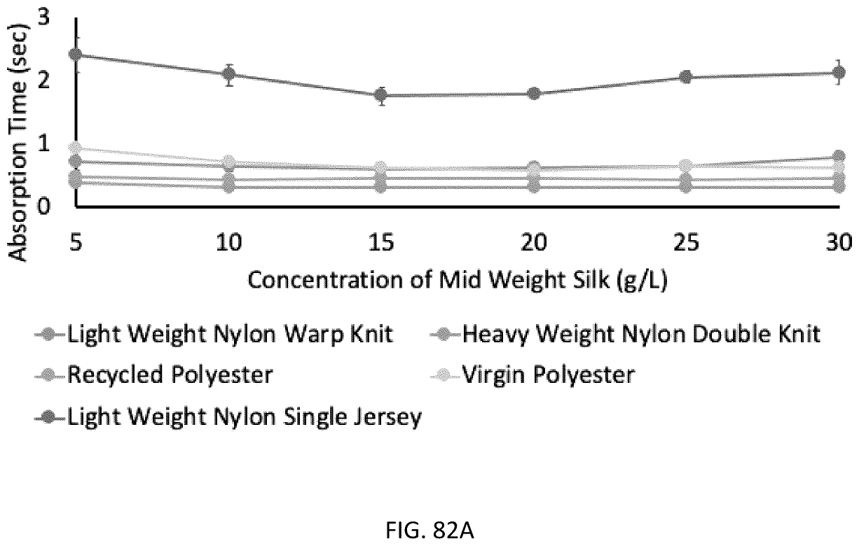

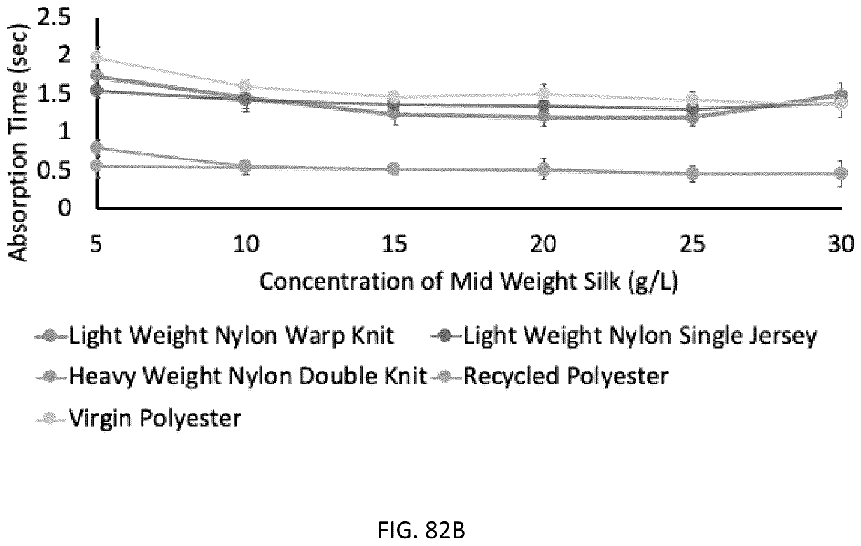

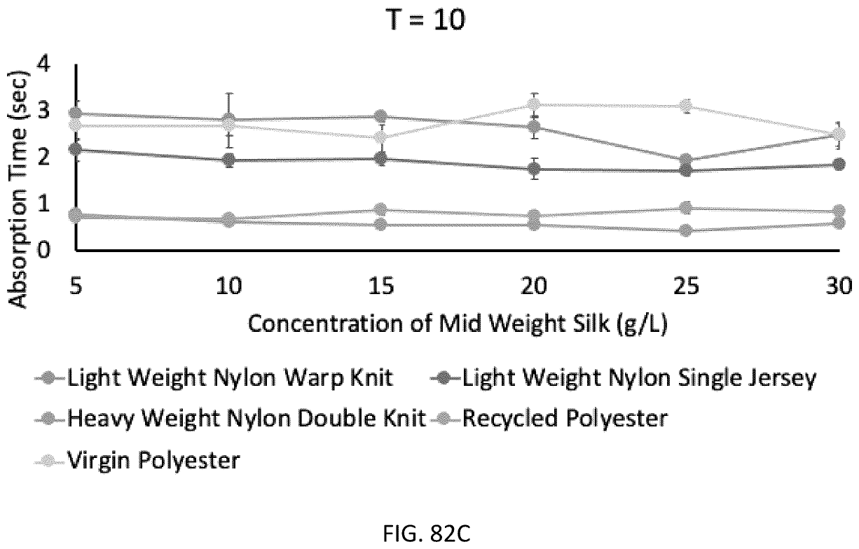

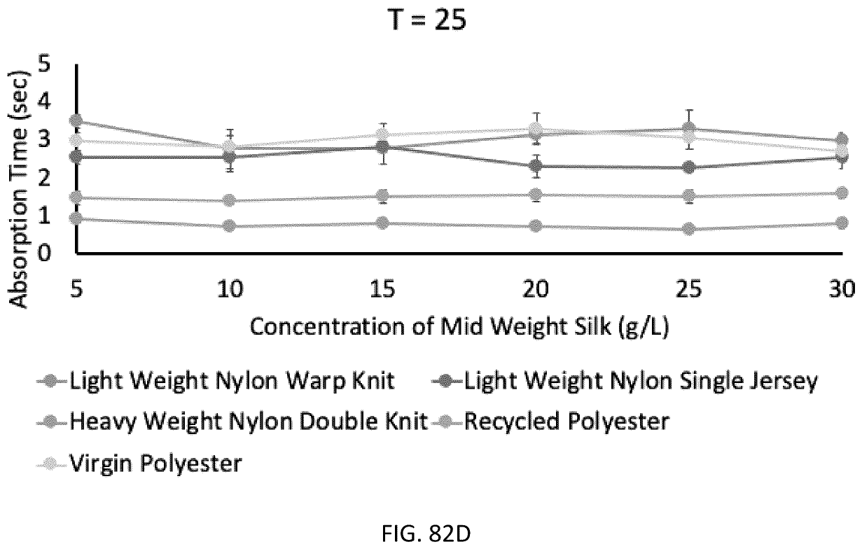

From the moisture management data, there is little variation in absorption time with an increase in mid weight silk. Without wishing to be bound by any particular theory, in general, the absorption times for mid weight silk are higher than their low weight counterpart. With the low molecular weight silk at 20 g/L, there is only one fabric which surpasses the 2 second cut off criteria. However, even at 0 washes there is still one fabric which passes this point. This behavior only becomes worse as the number of washes increases when comparing the same fabric between low and mid weight silk (FIGS. 82A-82D).

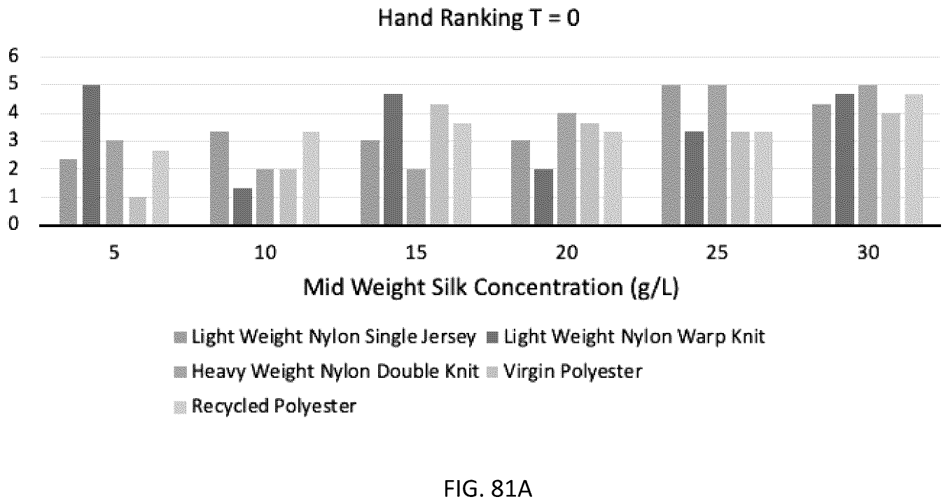

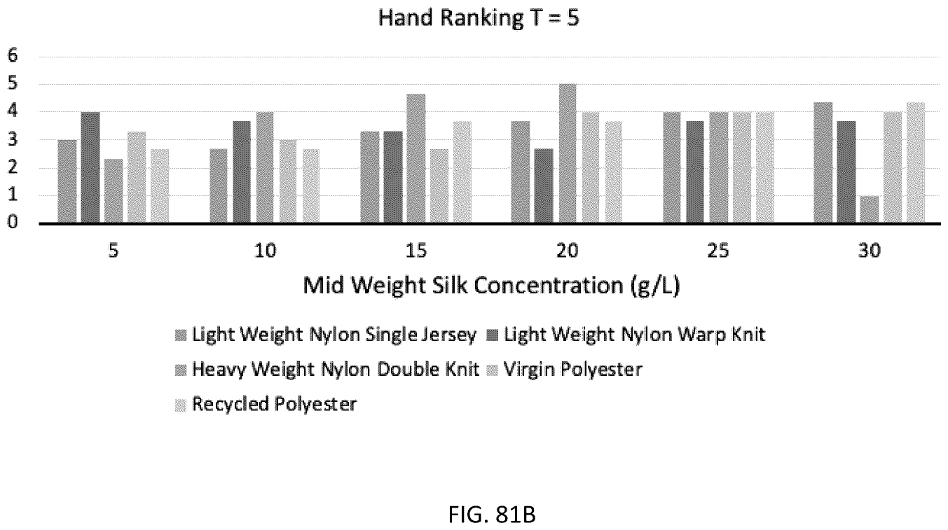

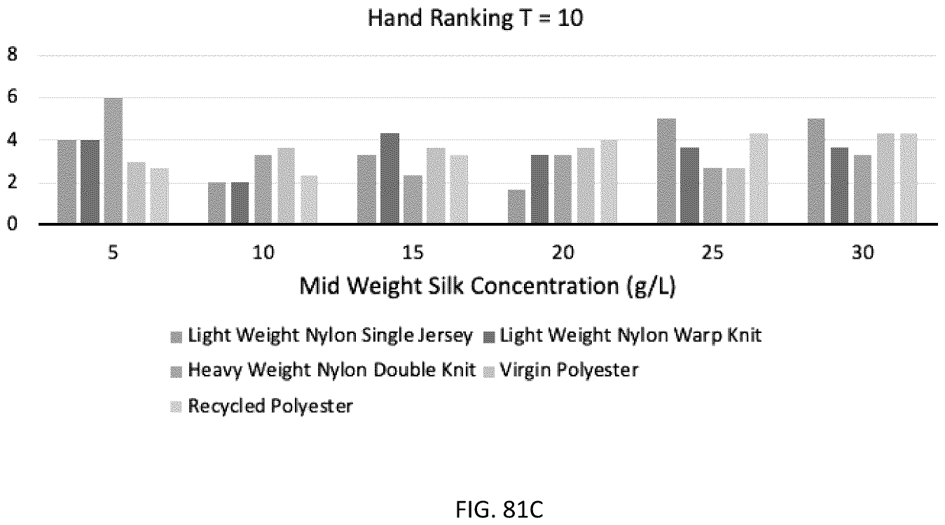

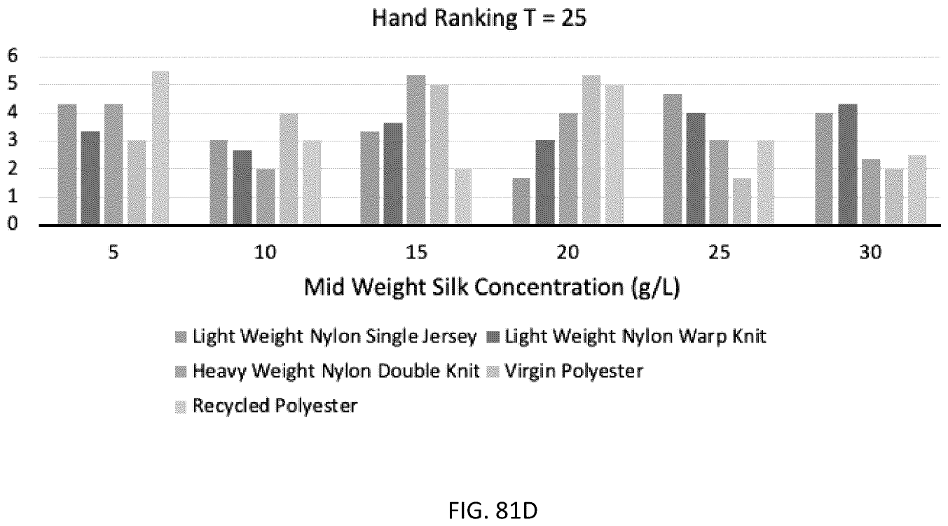

From a hand feel perspective, similar to the moisture management data, there does not appear to be a discernable trend with an increase in mid weight silk in the coating solution (FIGS. 81A-81D).

FIGS. 81A-81D are charts showing the hand feel ranking results from no washes (FIG. 81A), 5 washes (FIG. 81B), 10 washes (FIG. 81C), and 25 washes (FIG. 81D) generated by changing the concentration of mid molecular weight silk in the final coating solution; 1 is the best ranking and 8 is the worst ranking.

FIGS. 82A-82D are charts showing the moisture management results from no washes (FIG. 82A), 5 washes (FIG. 82B), 10 washes (FIG. 82C), and 25 washes (FIG. 82D) generated by changing the concentration of mid molecular weight silk in the final coating solution.

### Quantification of Silk on the Nylon Fabric

Using UV/Vis absorbency, the concentration of silk in solution can be measured by measuring the absorbance of the 276 nm peak of the silk spectrum. To quantify the amount of silk remaining on the fabric a standard curve was made through the serial dilution of a stock solution. After measuring the absorbance of each solution, a linear curve was fitted allowing for the conversion of solution absorbance to solution concentration. The silk can be removed from the silk coated fabric by sonicating the sample in a 9 M LiBr solution for 3 hours. The remaining silk solution can then be measured to quantify the amount of silk removed from the fabric. To determine the fiber surface area, microscopy was performed on filament cross sections of each fabric to obtain the fabric denier and subsequently calculate the surface area.

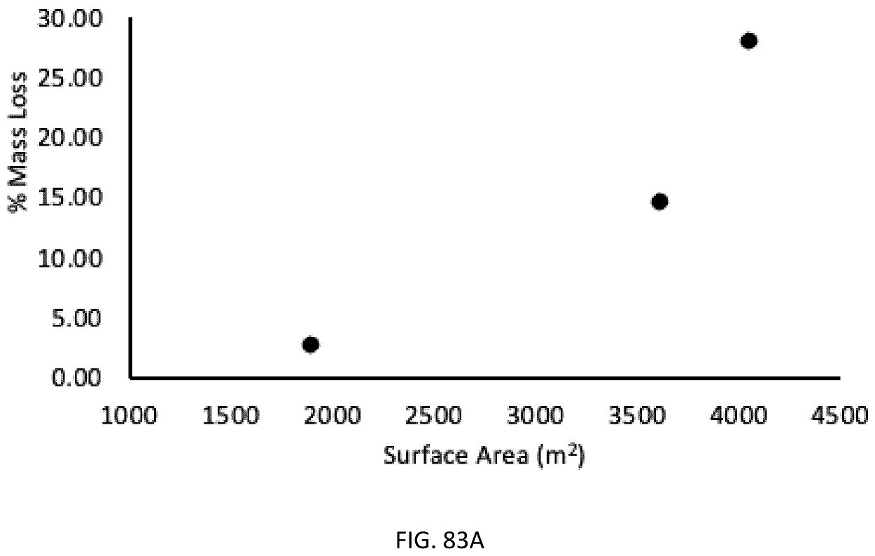

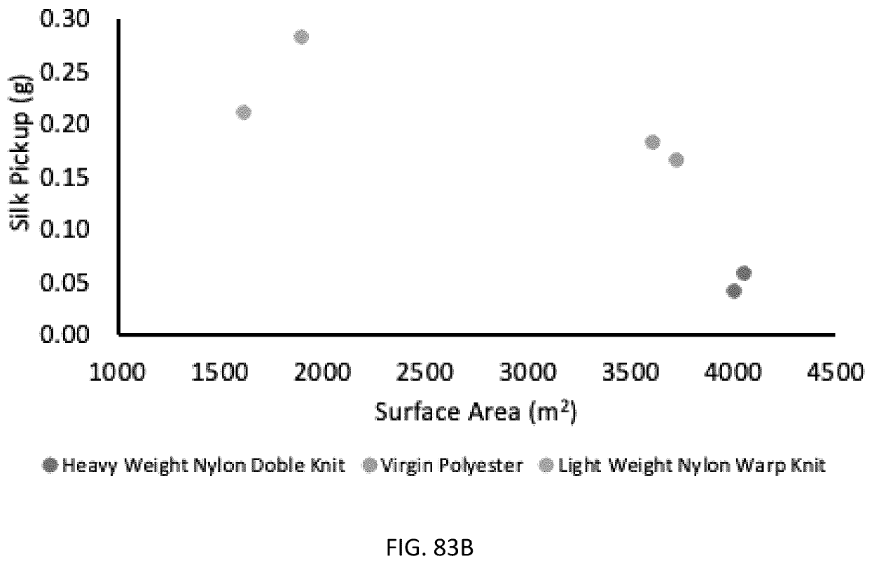

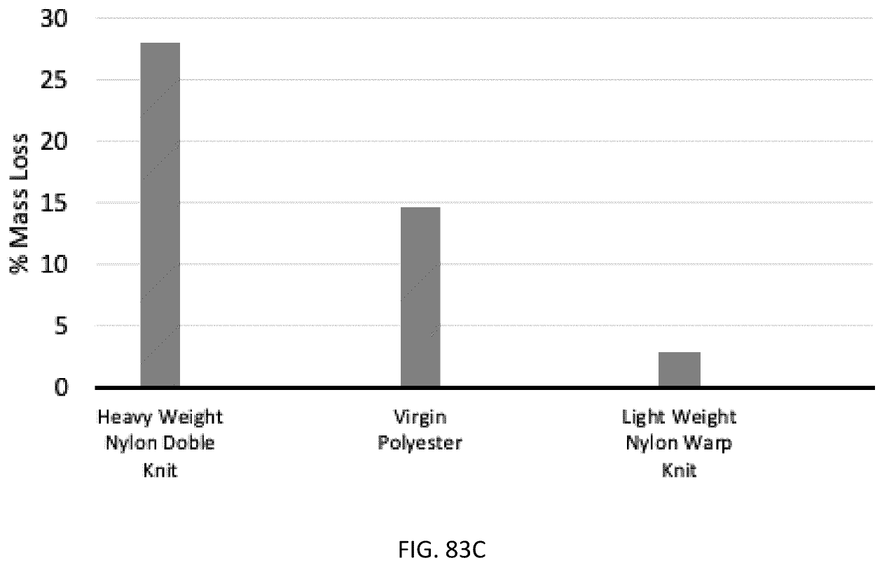

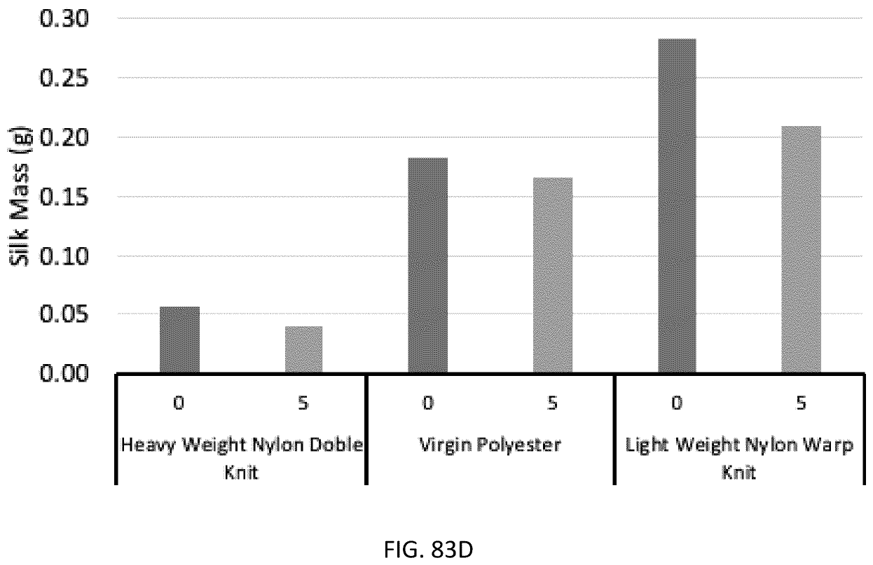

As can be seen in FIG. 83D, there is still silk on the fabric after washing, but the quantity has decreased. When comparing the percent of silk mass lost per sample, an increase in fiber surface area appears to increase the mass loss of silk from the fabric (FIG. 83A). However, the inverse can be said for silk uptake on the fabric. It appears that an increase in fiber surface area correlates to a lower uptake of silk on the fabric (FIG. 83B). Without wishing to be bound by any particular theory, there appears to be a correlation between silk adherence to the fabric and the surface of the fibers.

FIGS. 83A-83D are graphs showing UV/Vis quantification experiments of fabrics coated with low molecular weight activated silk and polyoxyethylene (20) monooleate solution. FIG. 83A: A graph showing the percent of silk lost after five washes with respect to fiber surface area. FIG. 83B: A graph showing the mass quantified of silk on the fabric after coating with respect to fiber surface area. FIG. 83C: A graph showing the percent of silk lost after five washes with respect to fabric type. FIG. 83D: A graph showing the mass of silk quantified on each fabric before and after five washes depending on fabric type.

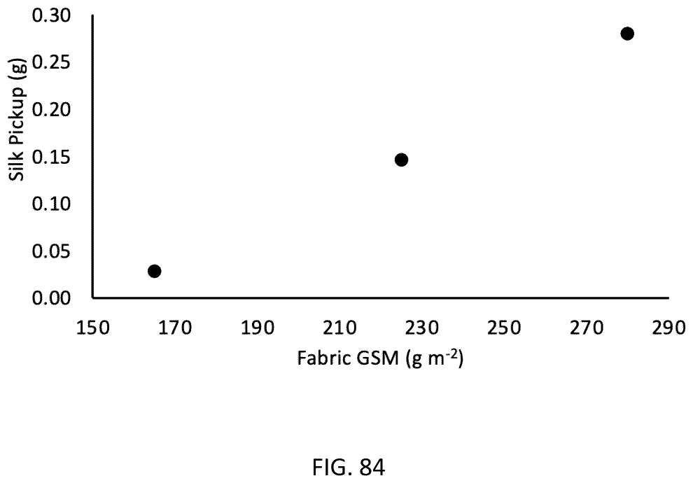

FIG. 84 is a chart showing UV/Vis quantification experiments of fabrics coated with low molecular weight activated silk and polyoxyethylene (20) monooleate solution. A graph showing the mass quantified of silk on the fabric after coating with respect to fabric mass in grams per square meter (GSM). This mass is dependent on knit type, fiber content, and filament denier.

### Silk Surface Charge Study

A potentiometric titration was done on silk coated nylon fabrics, Archroma RPU coated fabrics, and nylon fabric controls. Different nylon fabrics were coated in a solution of Activated Silk (20 g/L) and Polyoxyethylene (20) sorbitan monooleate (2 g/L) or in a solution containing 2% Archroma RPU liquid. A sample of each fabric was washed in a front-loading washing machine using an AATCC non softening non brightening detergent for five washes. For the titration process 0.1 M sodium chloride was used as the counter ion and hydrochloride acid or sodium hydroxide were used as the titrants.

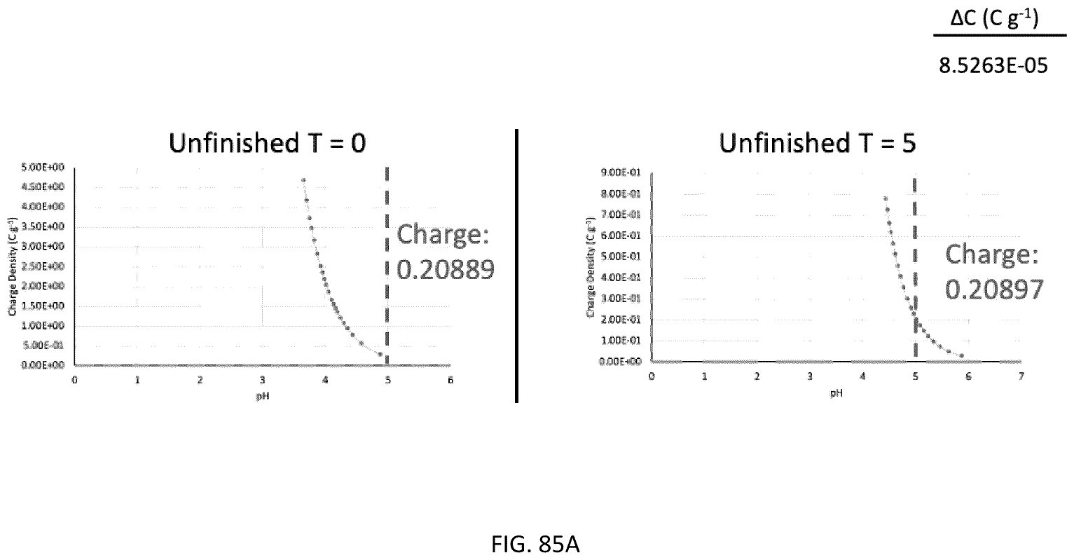

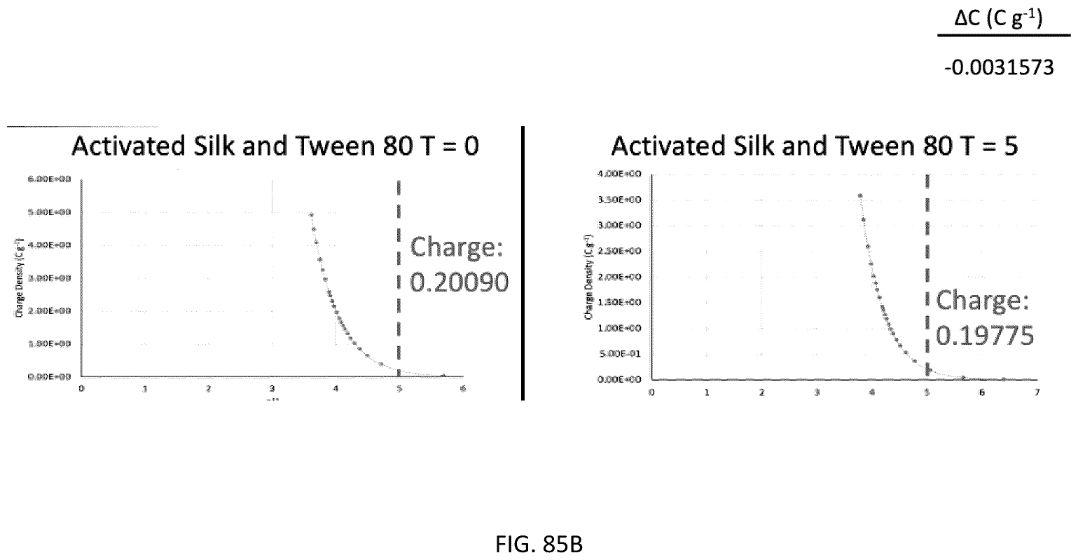

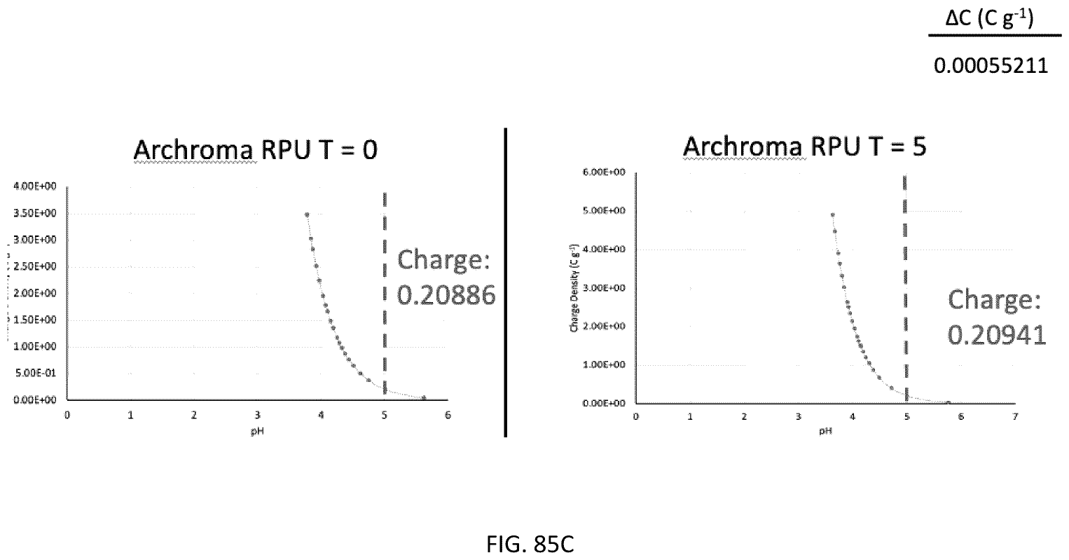

The resulting titration curves, as seen in FIGS. 85A-85C, show a more negative charge at pH 5 for silk coated fabrics (ΔC=−0.00316 C g⁻¹) than unfinished fabrics (ΔC=0.00008 C g⁻¹) or fabrics coated in Archroma RPU liquid (ΔC=0.00055 C g⁻¹). Without wishing to be bound by any particular theory, this shows that after washing silk coated surface is more negatively charged than the unfinished control or the positively charged Archroma RPU coated fabric.

FIGS. 85A-85C include a series of charts showing potentiometric titration curves with the charge density measured at a pH of 5 for the unfinished heavy weight double knit nylon fabric (FIG. 85A), activated silk finished heavy weight double knit nylon fabric (FIG. 85B), and Archroma RPU wetting agent finished heavy weight double knit nylon fabric (FIG. 85C). Each fabric has a titration curve obtained at no washes (FIGS. 85A-85C, left panels) and at five washes (FIGS. 85A-85C, right panels). The change in charge density at pH 5 after washing is denoted as ΔC.

### Example 18: Surface Charging Study

### Background

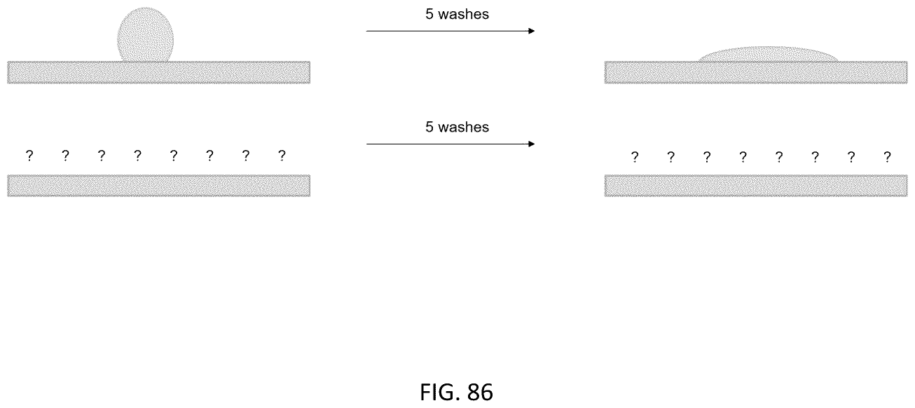

Based on previous washing performance data, it was proposed that silk can recharge the surface of a fabric after washing. In this study, that hypothesis was tested using potentiometric titration and zeta potential measurement of the surfaces of fabrics before and after washing to determine any surface charging. For this study, surface charging behavior is defined as a change in surface charge density at a set pH. For fabric recharging, samples of fabric were prepared and washed for five washes in a front-loading washer and surface charging was measured as the difference in charge before and after washing (FIG. 86) The detergent used for washing was an anionic solid surfactant mixture of sodium carbonate, sulfate, percarbonate, and sulfonate.

TABLE 43
List of samples tested for surface charging

| Fabric Style | Sample Coating | | |
|---|---|---|---|
| Midweight Nylon | Unfinished | AS 200 | Hydroperm RPU |
| Grey (Darlington G) | | | |
| Midweight Nylon | Unfinished | AS 200 | Hydroperm RPU |
| Black (Adidas YY) | | | |
| Heavyweight Nylon | Unfinished | AS 200 | Hydroperm RPU |
| Black (Sculptek Lite) | | | |
| Recycled Polyester | Unfinished | AS 200 | Hydroperm RPU |
| (RPP) | | | |
| Virgin Polyester | Unfinished | AS 200 | Hydroperm RPU |
| (Sculpt Polyester) | | | |

### Zeta Potential

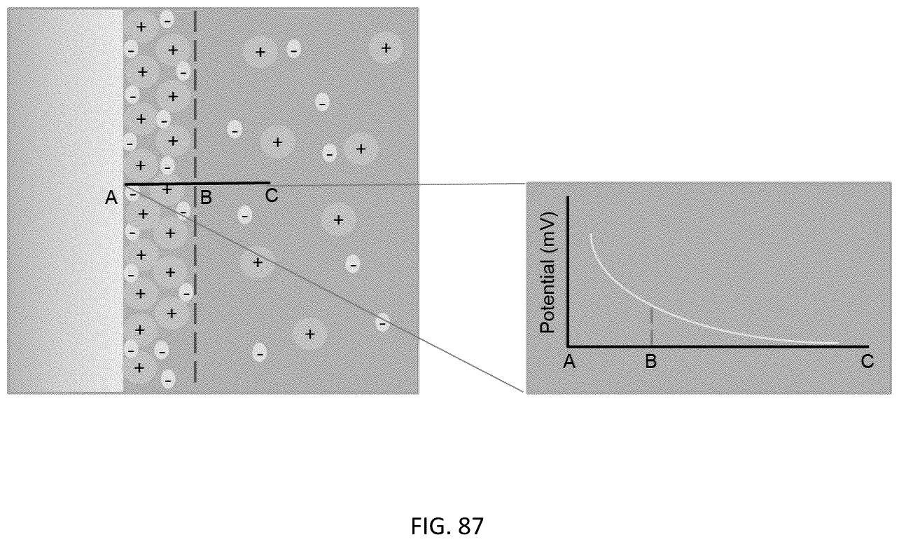

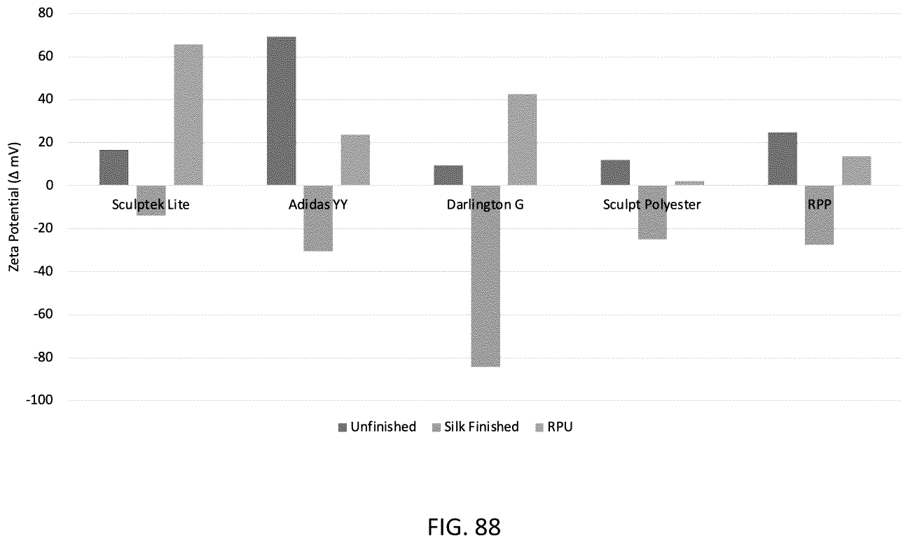

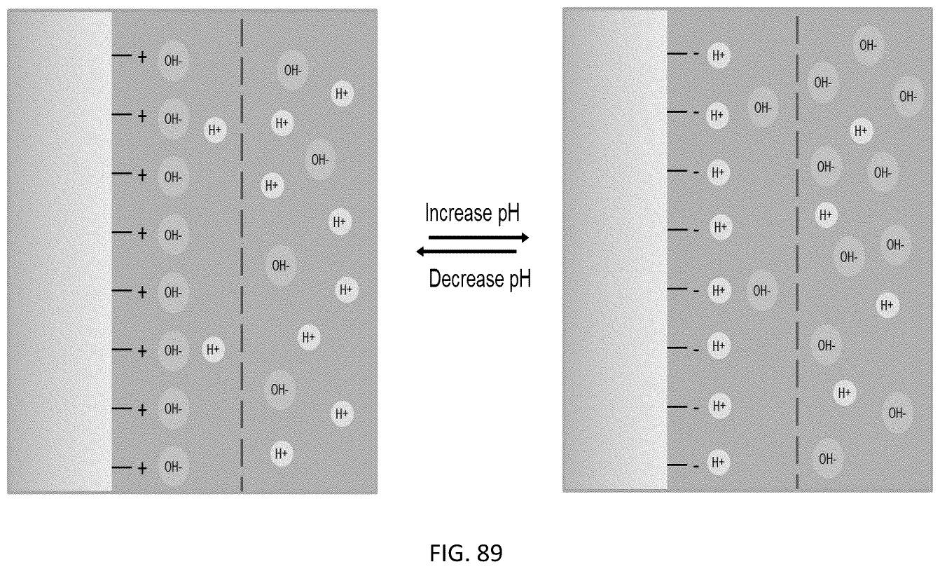

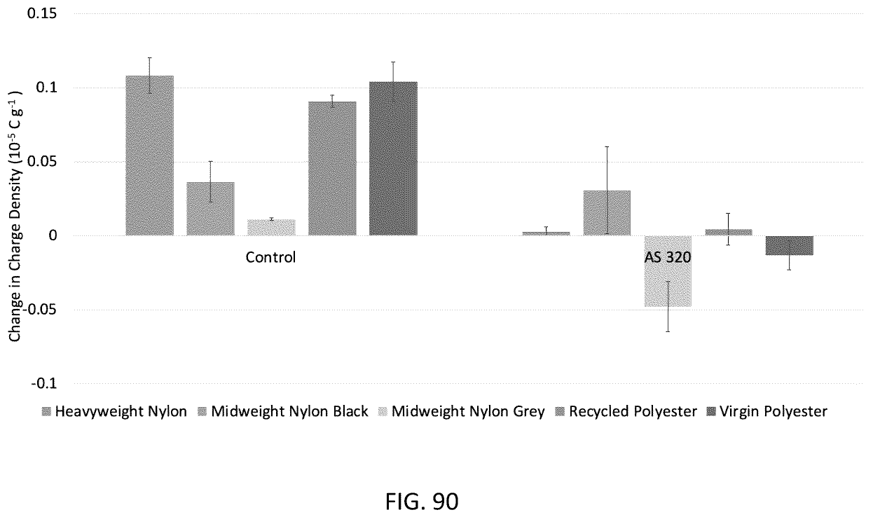

The Zeta Potential is the electrical potential at the interface of immobilized ions and solvated ions. This value is directly correlated to the net electrical charge of the plane of particles on a solid surface. By measuring the zeta potential, the charging behavior of a surface can be elucidated (FIG. 87). The change in surface charge for silk is always negative no matter the fabric construct or fiber content Both RPU and unfinished fabrics become more positively charged after washing, no matter the fabric.

### Potentiometric Titration

An alternative descriptor of surface charging is the concentration of charges found on a material's surface when titrated in the presence of a counter ion. Using potentiometric titration, the charging of the surface is directly correlated to the moles of protons and hydroxide ions adsorbed to the surface at a given pH.

The unfinished fabric has a larger change in surface charge than the silk finished fabric. The silk finished fabrics are the only fabrics with a negative change in surface charge.

### Conclusion

Based on these results, there is clearly a different charging behavior for silk coated fabrics compared to unfinished and RPU fabrics in response to washing. In some embodiments, during the washing process, silk is removed from the surface. This process leaves behind vacant sites with a negative charge giving the surface a greater negative charge than before.

In some embodiments, because of the amphoteric nature of silk, when washed under the basic conditions created by the detergent, the silk could become negatively charged after deprotonation. In some embodiments, during washing, the silk can absorb anions found in the detergent, creating a more negatively charged surface than before washing.

### Example 19: Silver Silk Nanoparticles to be Used as Antimicrobial Agents

The objective of this experiment is to evaluate the effectiveness of synthesized Silver Silk Nanoparticles to be used as an antimicrobial finishing agent for nylon.

Previous work in the field of antimicrobial technology has lead to the implementation of silver nanoparticles as an antimicrobial agents, due to silver's microbial toxicity. In this study silver nanoparticles were made using a silver nitrate solution and low molecular weight silk and nylon fabrics were coated to test for antimicrobial activity. In this study the nanoparticles were synthesized using a method defined in the study in question while coating in replacement of the standard low molecular weight silk solution.

### Materials

TWEEN 80
AS-304-LS
Unfiltered tap water
10% Acetic acid solution
Silver Nitrate
Nylon blend fabrics

### Equipment:

Werner Mathis MA0881 padder/coater
VWR Standard Magnetic Stirrer (120V)
Balance Veritas M314-AI
Orion Star A221 pH Portable Meter
Buffer pH Standard solutions, 4,7,10
Across International Oven FO-19140
Glassware
   500 mL Beaker
   1 L Beaker
Stir Bars
Pipettes
   5 mL Pipette
   10 mL Pipette

### Methods

a) Dilute 304-LS to a 1% solution in water
b) Add 40 mg of AgNO₃ to the 304-LS solution
c) Let solution sit for more than 24 hrs until it becomes dark brown.
d) Cut test fabrics into 7.5″×6.5″ pieces
e) Create a diluted acetic acid solution by adding 0.84 g of acetic acid to a beaker of unfiltered tapwater.
f) Add the proportional amount of each component of the system and then mix for more than 30 seconds
g) First add TWEEN Molecule, 0.80 g
h) Finally, silver nanoparticle silk solution 5.00 g
i) Soak a sample of nylon fabrics into the sample solution and heat pad under 25 pressure rating and a 10 speed rating
j) Heat samples in an oven under 160° C. for 2 minutes to 3 minutes.
k) Allow samples to cool overnight.

TABLE 44
Antimicrobial Silver: Silk Sample Name.

| Mass of Silver:Silk Solution | |
|---|---|
| Added (mg) | Sample Name |
| 40 | SAgNPs |

TABLE 45
Solution Prep:

| Ingredients | Quantity (g) |
|---|---|
| Solution (mL) | 400.000 |
| AA 10% | 0.840 |
| Tween 20 (g) | 0.800 |
| Ag NPs (g) | 5.000 |
| Silk 304 (mL) | 6.670 |
| Water (mL) | 391.690 |

### Characterization

The coated samples were characterized by their antimicrobial efficacy. The test that will be performed is the Parallel Streak method “AATCC TM147-2011 (2016)e: Test Method for Antibacterial Activity of Textile Materials”

### Testing Information:

Staphylococcus aureus ATCC 6538
Klebsiella pneumoniae ATCC 4352
Sample size: 25×50 mm
Sterilization: none
Incubation: 37+/−2° C. for 18-24 hrs
Cultures stored at 5°+/−2° C.

### Results:

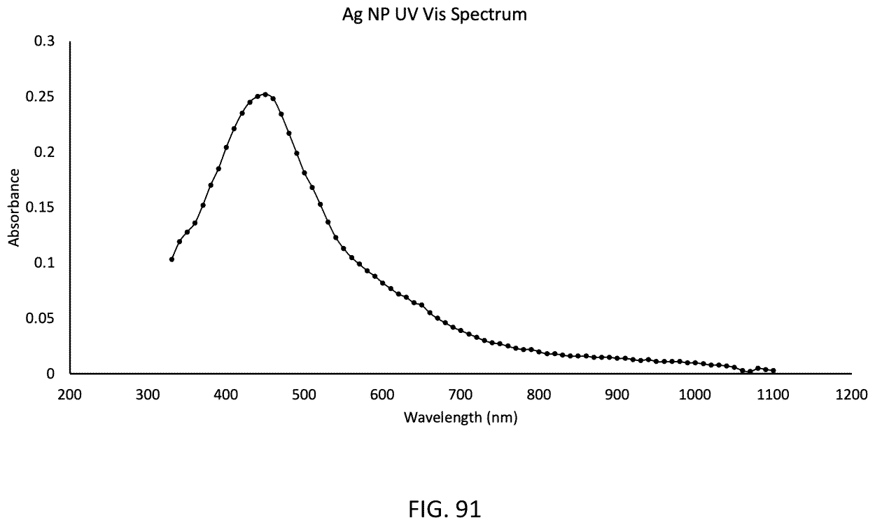

The UV/Vis spectrum of the synthesized silver nanoparticles was consisted with the particles synthesized in the paper (FIG. 91).

The streak test was performed on the adidas fabric at T=0 and at T=5 shown no antimicrobial growth on the face of the fabric. At T=5 there was a 2 mm zone of antimicrobial growth inhibition, suggesting possible leaching of the nanoparticles.

TABLE 46
Parallel Streak Test Result.

| Sample | Organism Growth on Surface | Zone of Inhibition |
|---|---|---|
| 0 washes | none | none |
| 5 washes | none | 2 mm |

TABLE 47
Parallel Streak Test Result for Specific
Bacteria (no zone of inhibition).

| | Face Side Down | Zone of Inhibition |
|---|---|---|
| S. aureus | No growth | none |
| K. pneumoniae | No growth | none |

TABLE 48
Parallel Streak Test Result for Specific
Bacteria (2 mm zone of inhibition).

| | Face Side Down | Zone of Inhibition |
|---|---|---|
| S. aureus | No growth | 2 mm |
| K. pneumoniae | No growth | 2 mm |

### Conclusion

From the results of this study, it was shown the synthesis of silver nanoparticles from low molecular weight silk is possible as well sustained antimicrobial activity before and after washing.

### Example 20: Cationic Fixing Agent Pretreated Recycled Nylon

The objective of this study is to characterize AS-320 coated recycled nylon, pretreated with a cationic fixing agent with absorbency from T=0 to T=10.

### Materials

ECO TWEEN 80, distributed by EVB, Lot #STI-LAB-149-40
EBV 14A (reconstituted to 5%)—Lot #21342-01
Unfiltered tap water
10% Acetic acid solution
Cationic Fixing Agent
Nylon blend fabrics:
   DC95-FA/#62701078/RN035Y3-BL-L1/1-000/80%/a Recycled Nylon, 20% Lycra/Black/Tricot/190gsm/32g gauge
   DC98-IA/RN035Y4-BL-L1/1-000/80%/a Recycled Nylon; 20%
Lycra/Night Blue/Tricot/212gsm/32 g gauge

### Equipment:

Werner Mathis MA0881 padder/coater
VWR Standard Magnetic Stirrer (120V)
Balance Veritas M314-AI
Orion Star A221 pH Portable Meter
Buffer pH Standard solutions, 4,7,10
Across International Oven FO-19140
Glassware
   500 mL Beaker
   1 L Beaker
Stir Bars
Pipettes
   5 mL Pipette
   10 mL Pipette

### Methods

a. Cut fabrics in 7.5″×6.5″ pieces
b. Add cationic fixing agent 8 g/L to tap water. Soak a sample into the solution for 1 minute and pad under 35 pressure rating with a 3m/min rating. Dry the sample in an oven under 130° C. for 2.5 minutes. Let samples cool for at least 30 minutes.
c. Add the proportional amount of each component of the system and the mix for more than 30 seconds
   First add Acetic Acid
   Add ECO TWEEN 80 2 g/L or 3 g/L
   Add 14A 15 g/L, 20 g/L, or 30 g/L
   Adjust the pH with acetic acid to 4.3-4.5.
d. Soak a sample of fabrics into the sample solution and heat pad under 35 pressure rating and a 3m/min rating.
e. Dry the sample in an oven under 130° C. for 2.5 minutes.
f. Allow samples to cool overnight.

TABLE 49
Sample ID of Recycled Nylon Fabrics

| Sample ID | Reference # | AS-320 Recipe |
|---|---|---|
| 685-C-41 | Black | Cationic fixing agent pretreated 8 g/L; |
| | DC95-FA | 14a 15 g/L + Eco Tween 80 2 g/L at |
| | | pH 4.3-4.5 |
| 685-C-49 | Black | Cationic fixing agent pretreated 8 g/L; |
| | DC95-FA | 14a 20 g/L + Eco Tween 80 2 g/L at |
| | | pH 4.3-4.5 |
| 685-C-43 | Black | Cationic fixing agent pretreated 8 g/L; |
| | DC95-FA | 14a 30 g/L + Eco Tween 80 3 g/L at |
| | | pH 4.3-4.5 |
| 685-C-71 | Night Blue | Cationic fixing agent pretreated 8 g/L; |
| | DC98-IA | 14a 15 g/L + Eco Tween 80 2 g/L at |
| | | pH 4.3-4.5 |
| 685-C-79 | Night Blue | Cationic fixing agent pretreated 8 g/L; |
| | DC98-IA | 14a 20 g/L + Eco Tween 80 2 g/L at |
| | | pH 4.3-4.5 |
| 685-C-73 | Night Blue | Cationic fixing agent pretreated 8 g/L; |
| | DC98-IA | 14a 30 g/L + Eco Tween 80 3 g/L at |
| | | pH 4.3-4.5 |

### Results:

Absorbency Pretreating recycled nylon (Black DC95-FA and Night Blue DC98-IA) with cationic fixing agent in general resulted in an improved absorbency of fabrics at T=0 and T=5 (except Night Blue DC98-IA at T=5). In addition, pretreatment with cationic fixing agent helped silk stick on recycled nylon fabrics since the absorbency of AS-320 coated pretreated recycled nylon maintained below 3 seconds from T=0 to T=10, which is a great improvement from the previous results in TXTL-655 Rev C pre-trial coating.

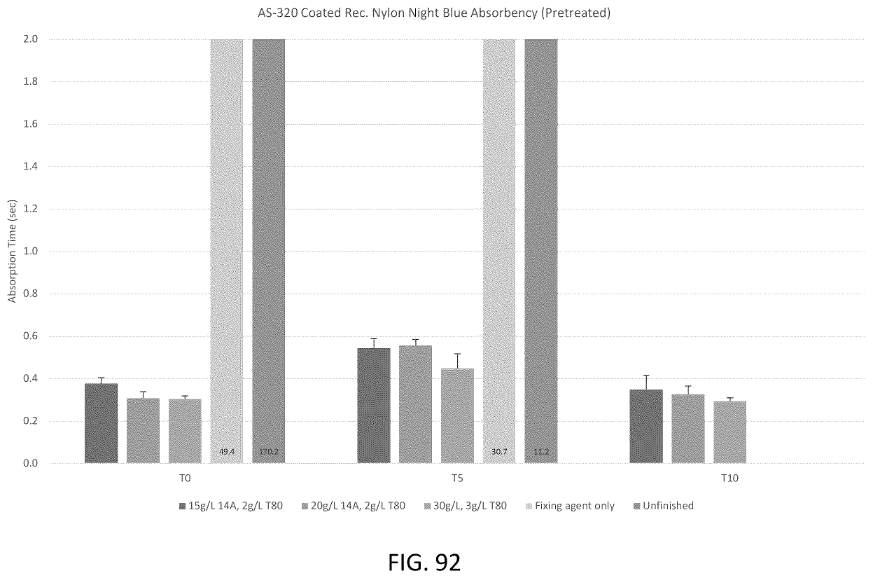

For Night Blue DC98-IA, with the cationic fixing agent pretreatment, AS-320 coated DC98-IA was able to maintain its absorbency below 1 second from T=0 to T=10. Among the three concentrations of AS-320, coating at 30 g/L 14A, 3 g/L EcoT80 gave the best absorbency from T=0 to T=10 (FIG. 92).

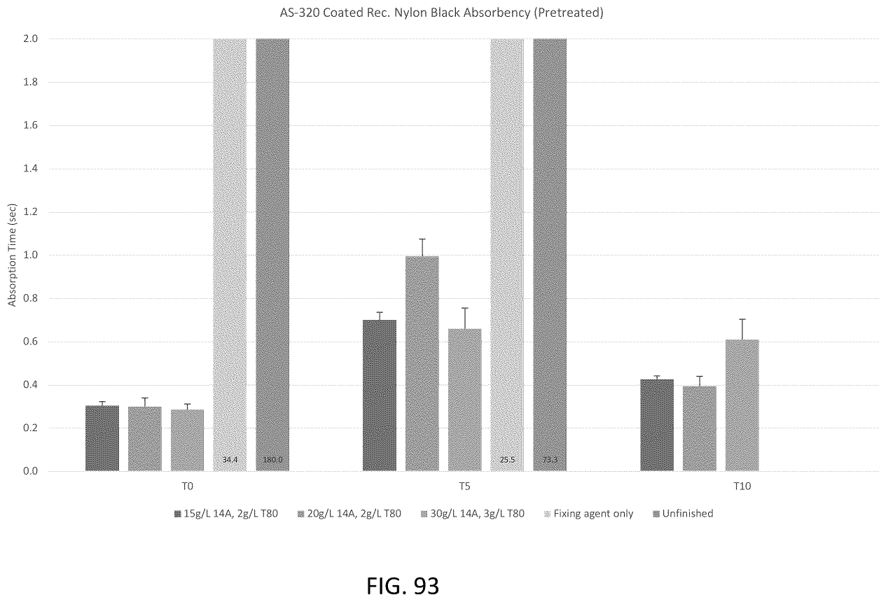

For Black DC95-FA, with the cationic fixing agent pretreatment, AS-320 coated DC95-FA was able to maintain its absorbency below 1 second from T=0 to T=10 (FIG. 93).

The worst absorbency of both fabrics was at T=5. However, the increase in absorbency was small enough to maintain the absorbency below or equal to 1 second (FIGS. 92 and 93).

From this study, it can also be concluded that the improved absorbency compared to TXTL-655 RevC is the contribution of both the cationic fixing agent and AS-320 since applying only the fixing agent on both fabrics did not result in an absorbency below 3 seconds.

### Example 21: Recycled Polyester AS-320 Coating with Fixing Agent Pretreatment

The objective of this experiment was to characterize the recycled polyester fabrics from coated with standard AS-320 cationic fixing agent pretreatment in-house at T=0,5, and 10.

Fabrics were recoated with fresh reconstituted silk and tested absorbency at T=0, 5, and 10. In this study, same fabrics were first pretreated with cationic fixing agent, followed by standard AS-320 coating to confirm whether the pretreatment could improve the moisture management properties of the coated fabrics.

### Materials

ECO TWEEN 80 Croda, Lot #STI-LAB-149-40
AS-304-IS (reconstituted to 5%)—Lot #TS-21342-01
Unfiltered tap water
10% Acetic acid solution
Polyester blend fabrics:
   17120513 Recycled Poly Bare
   Recycled Poly Bare 223412 INTERLOCK Polyester/BLACK lycra 75/25 240gsm 1.27 1.53 96.2/95.4 wicking finish only Finished=Dyed Black/Unfinished=Dyed black
   17120511 Recycled Poly 2.0 32 gg (poly)
   REC Poly 2.0 32 gg 223936 SINGLE JERSEY Poly/Lycra 88/12 278gsm 1.74 1.423 93.1%/95% wicking finish only Finished=Dyed Blue/Unfinished=Dyed Blue

### Equipment:

Werner Mathis MA0881 padder/coater
VWR Standard Magnetic Stirrer (120V)
Balance Veritas M314-AI
Orion Star A221 pH Portable Meter
Buffer pH Standard solutions, 4,7,10
Across International Oven FO-19140
Glassware
   500 mL Beaker
   1 L Beaker
Stir Bars
Pipettes
   5 mL Pipette
   10 mL Pipette

### Methods

a) Cut fabrics in 7.5″×6.5″ pieces
b) Add the proportional amount of each component of the system and the mix for more than 30 seconds
c) First add Acetic Acid
d) Add ECO TWEEN 80 2 g/L
e) Finally add AS 304-IS 15 g/L
f) Adjust the pH to 4.3-4.5
g) Soak a sample of fabrics into the sample solution and heat pad under 60 pressure rating and a 3m/min rating.
h) Heat samples in an oven under 130 C for 3 minutes.
   Allow samples to cool overnight.

TABLE 50
Sample ID of recycled polyester fabrics

| Sample ID | Reference # | AS-320 Recipe |
|---|---|---|
| 678-C-74 | 17120513 (black) | Fixing Agent 8 g/L PRETREATED |
| | | AS-304 15 g/L + Eco Tween 80 2 g/L |
| 678-C-75 | 17120513 (black) | Fixing Agent 8 g/L |
| 678-C-94 | 17120511 (blue) | Fixing Agent 8 g/L PRETREATED |
| | | AS-304 15 g/L + Eco Tween 80 2 g/L |
| 678-C-95 | 17120511 (blue) | Fixing Agent 8 g/L |

### Results:

### Absorbency

For both 17120513 black and 17120511 blue, the standard AS-320 coated fabrics (15 g/L 14A, 2 g/L EcoTween80) with cationic fixing agent pretreatment passed absorbency test from T=0 to T=10.

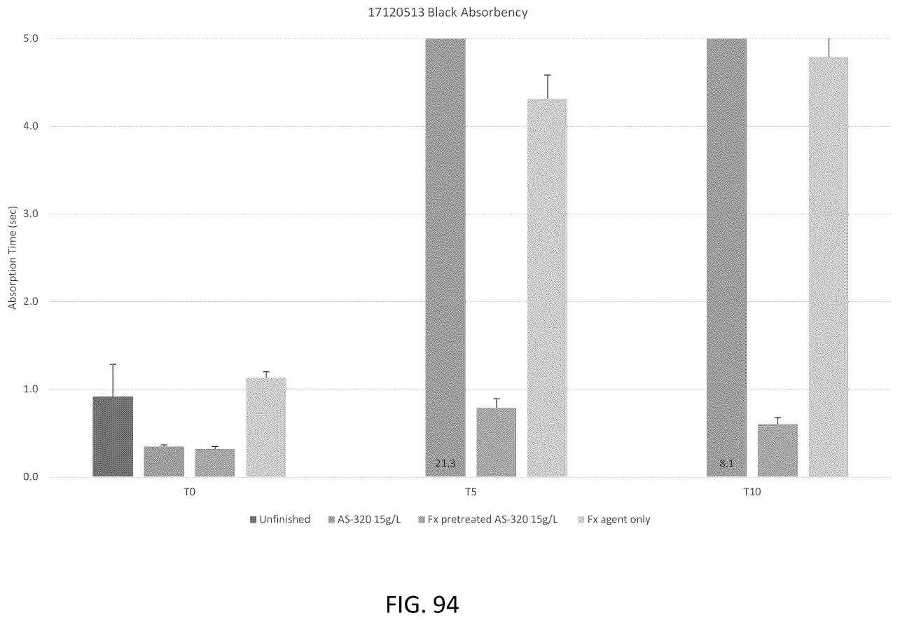

For 17120513 black, the standard AS-320 coated fabric had an absorbency more than 20 seconds at T=5 and around 8 seconds at T=10 based on TXTL-678 RevB. However, with HT-232H fixing agent pretreatment, the absorbency of the AS-320 coated fabric can maintain at below 1 second from T=0 to T=10. The low absorption time came from the contribution of both AS-320 and the fixing agent. With only HT-232H fixing agent treatment, the fabric had an absorbency between 4 to 5 seconds at T=5 and T=10 (FIG. 94).

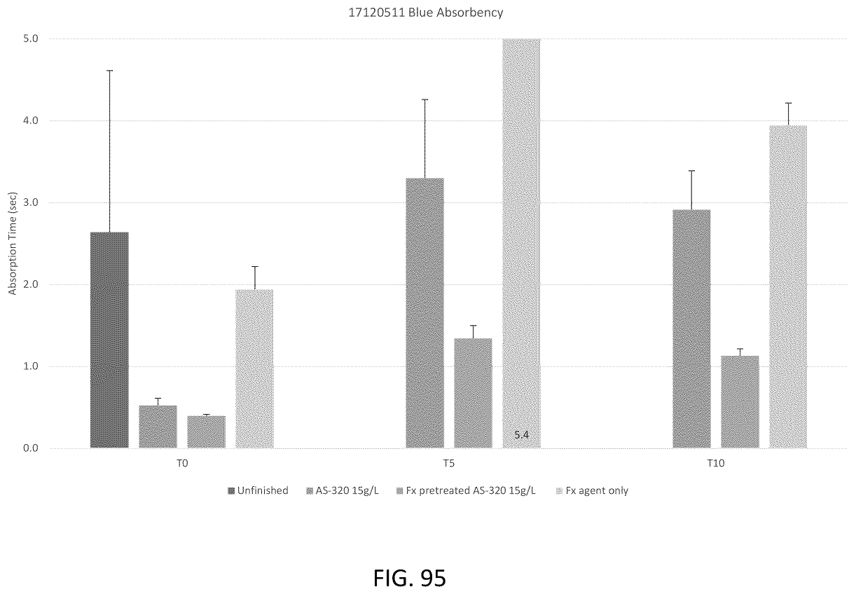

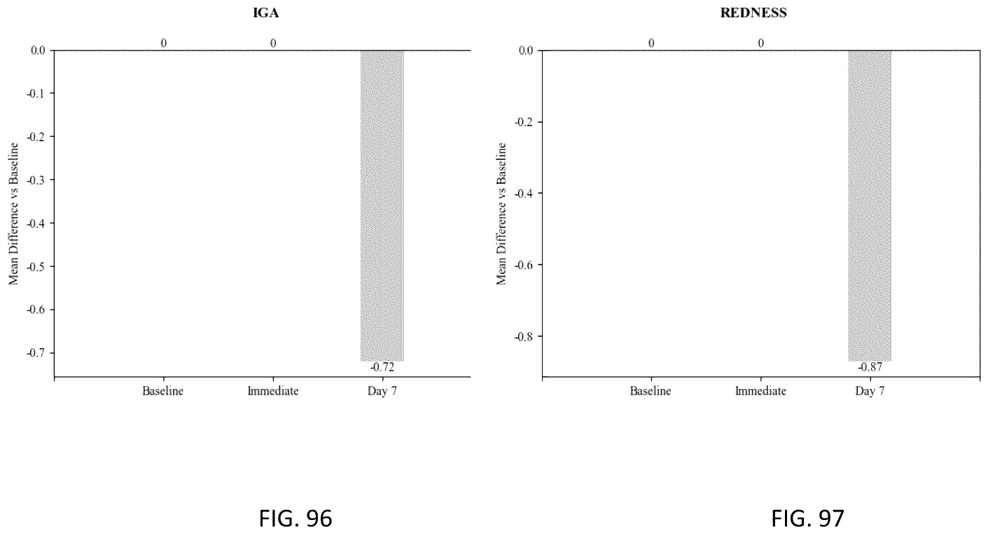

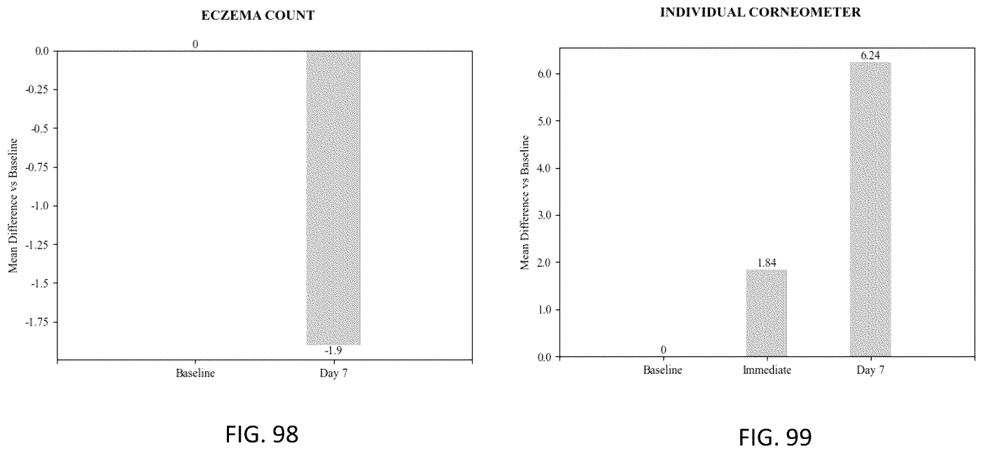

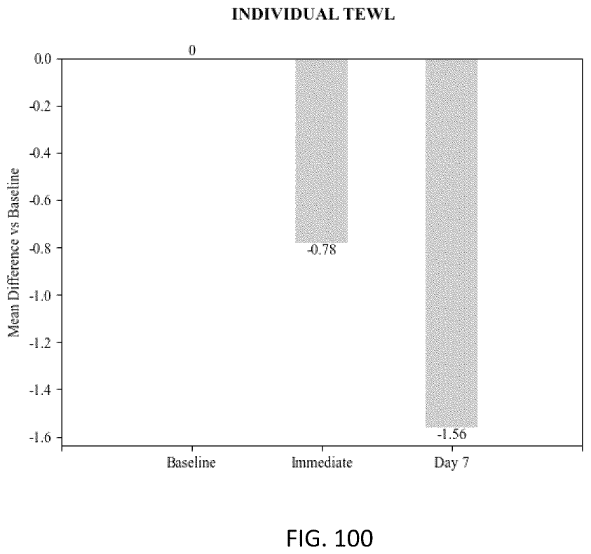

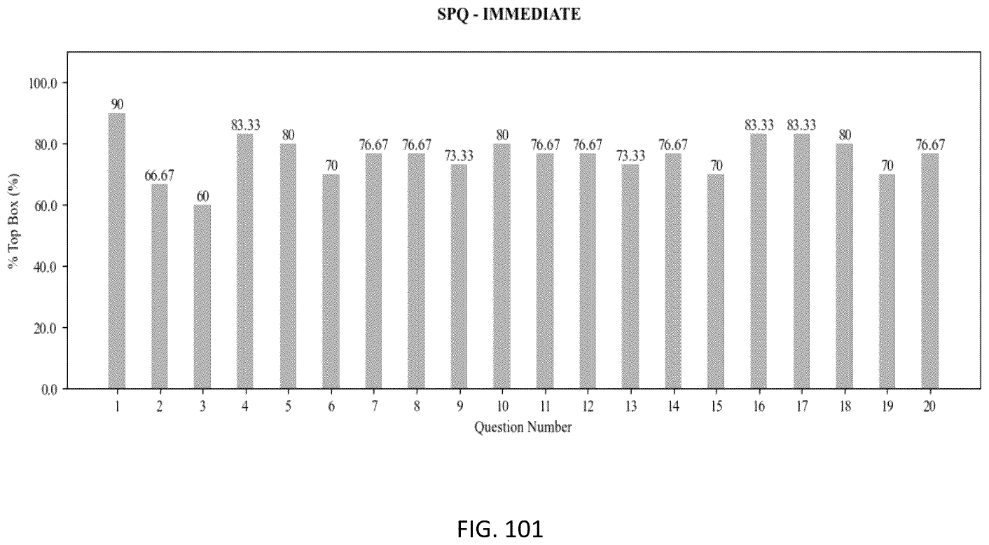

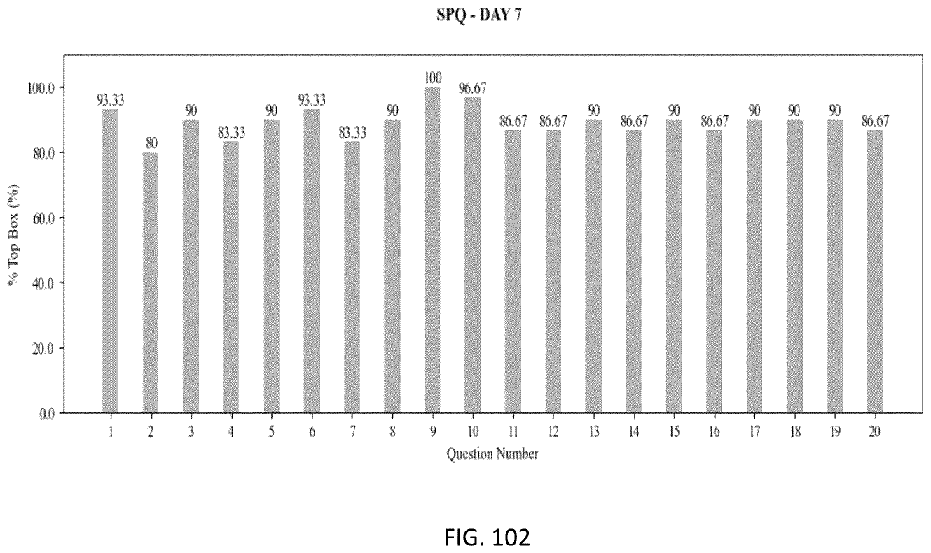

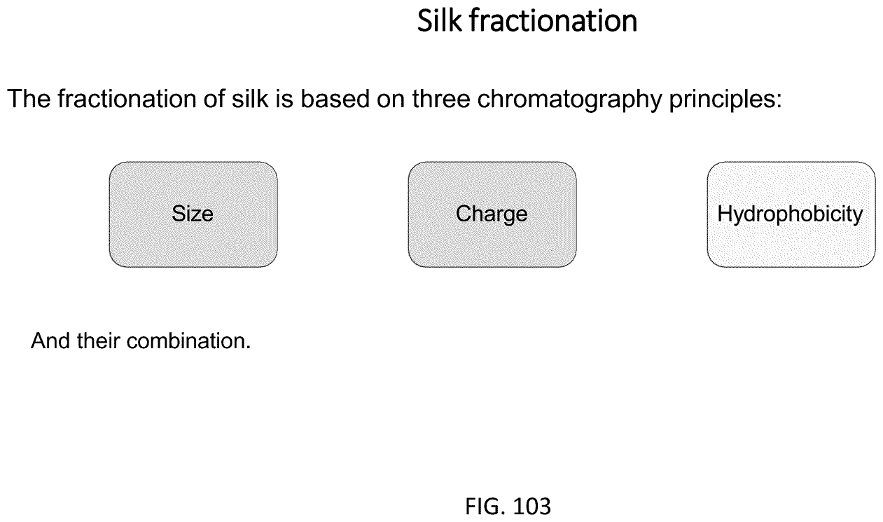

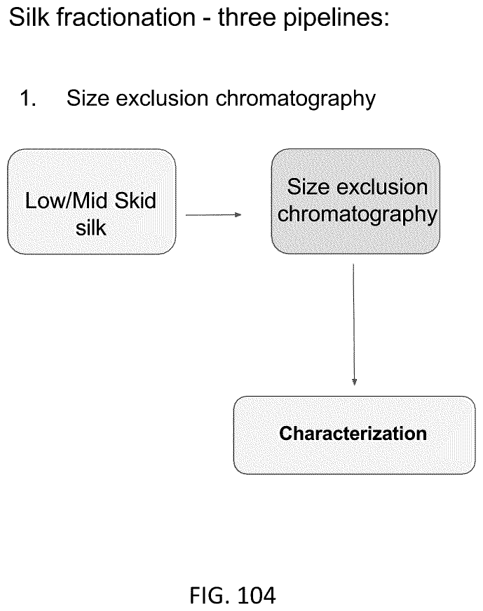

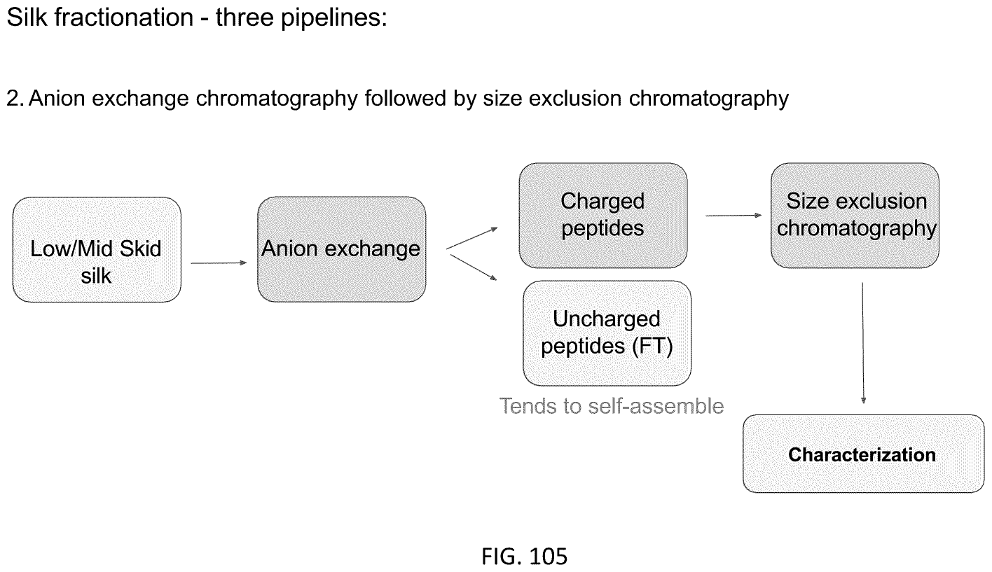

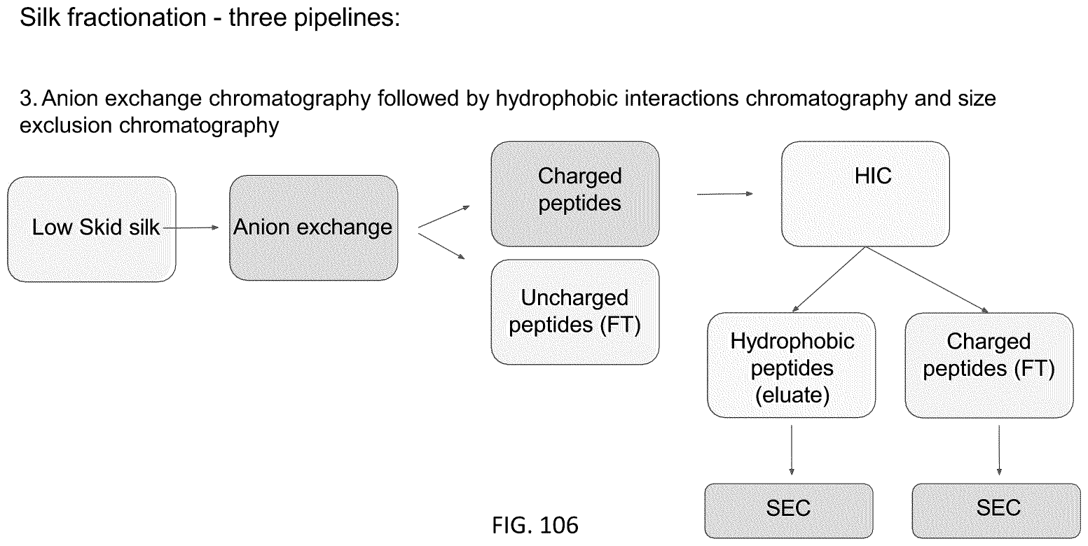

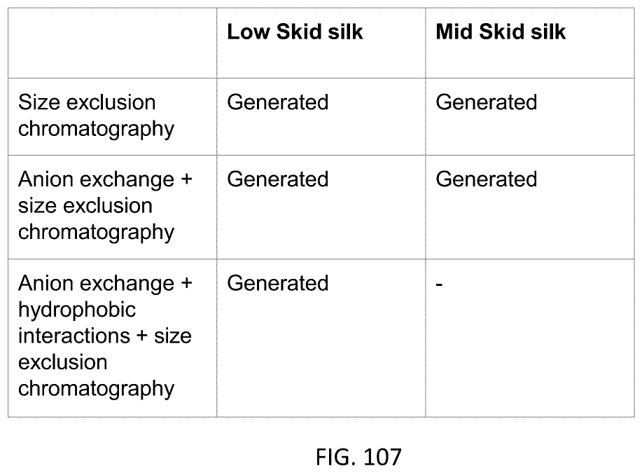

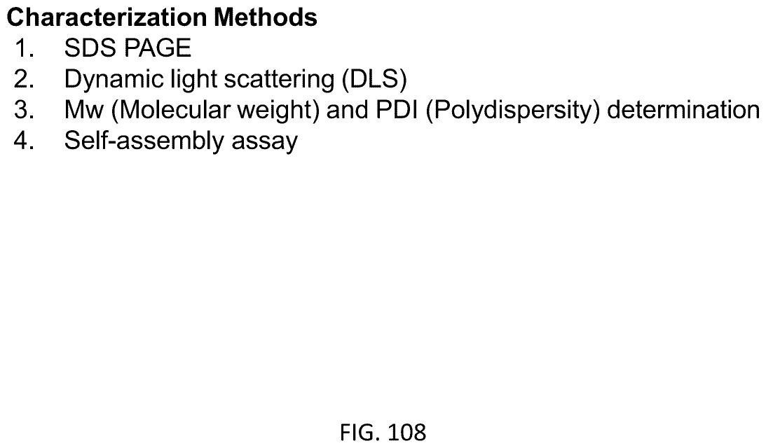

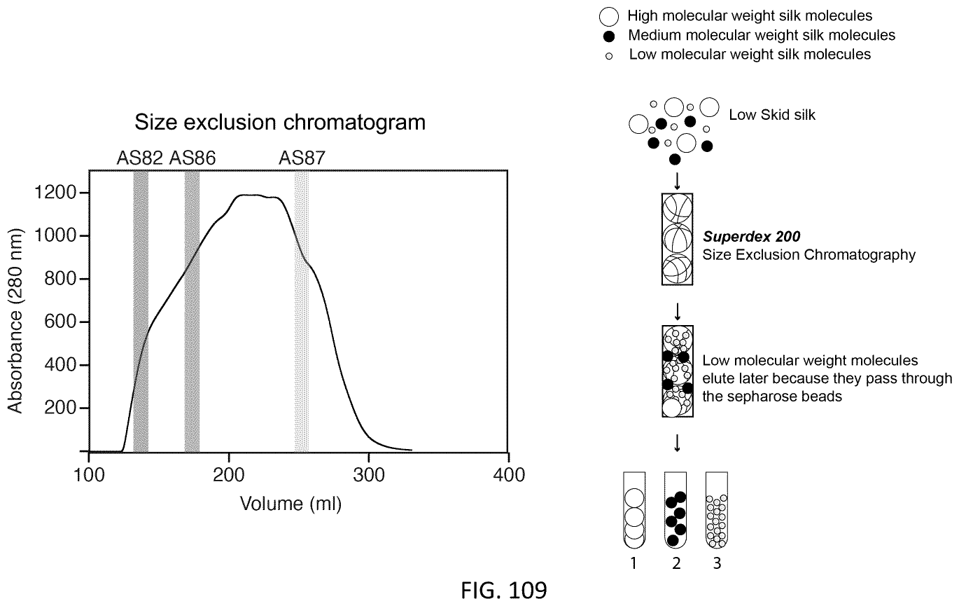

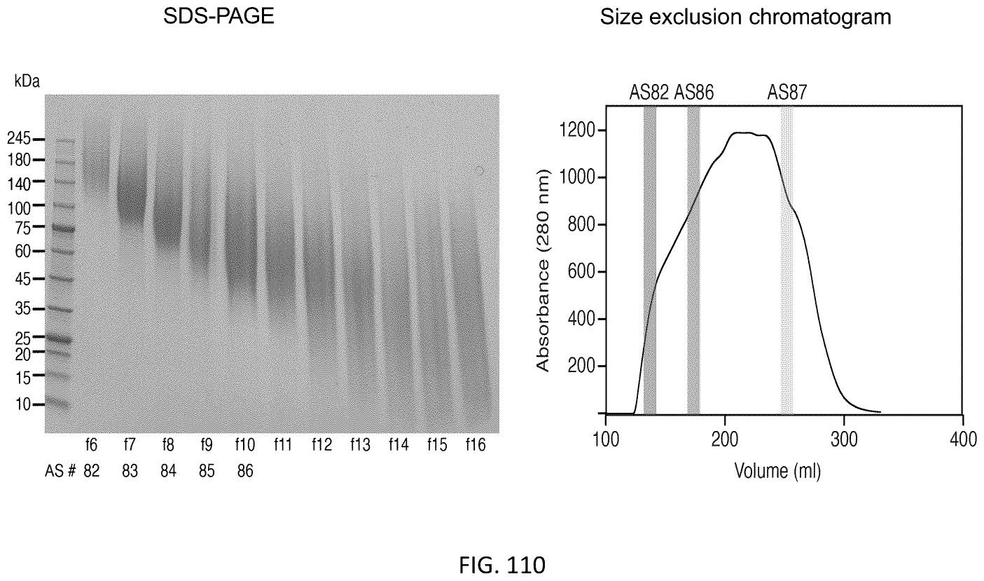

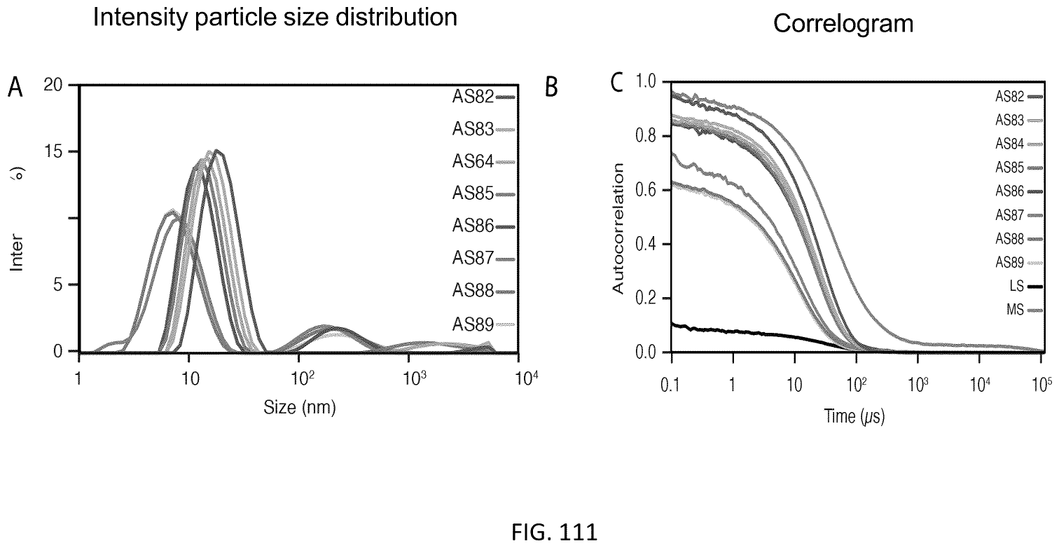

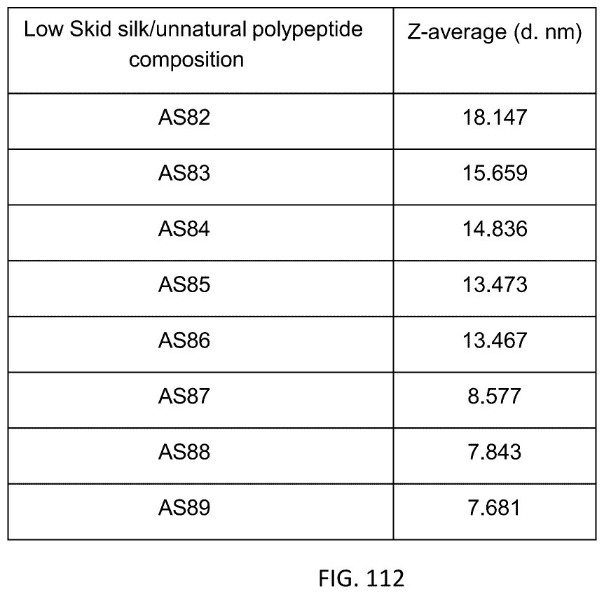

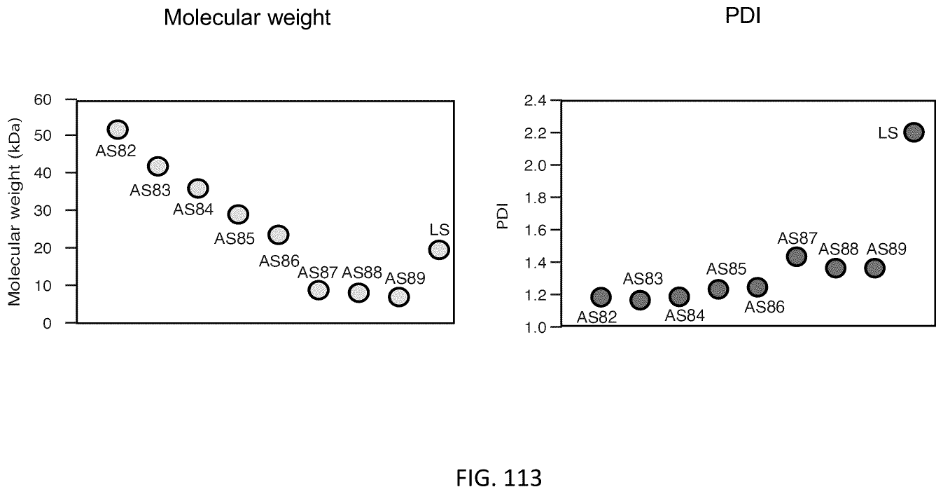

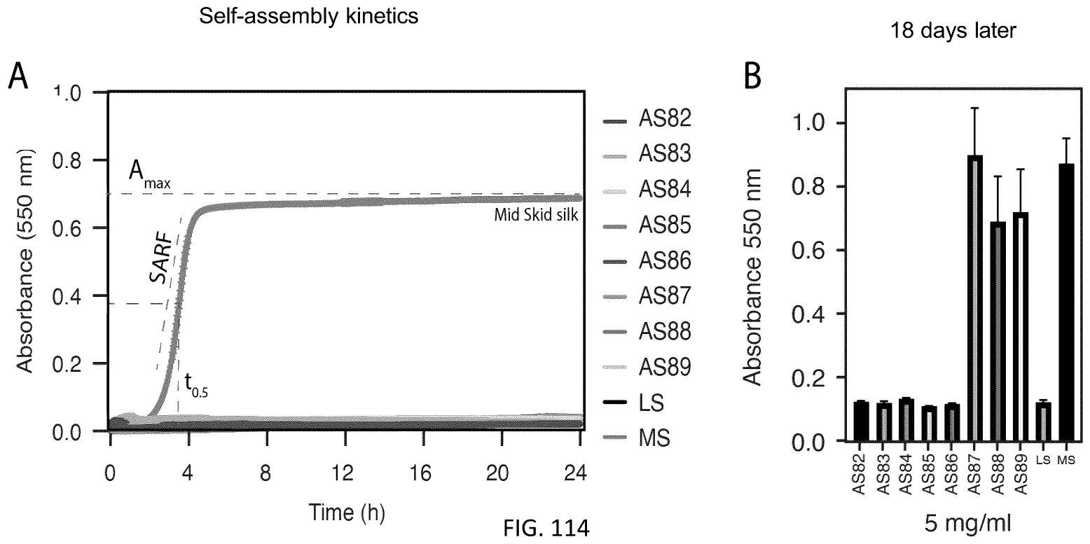

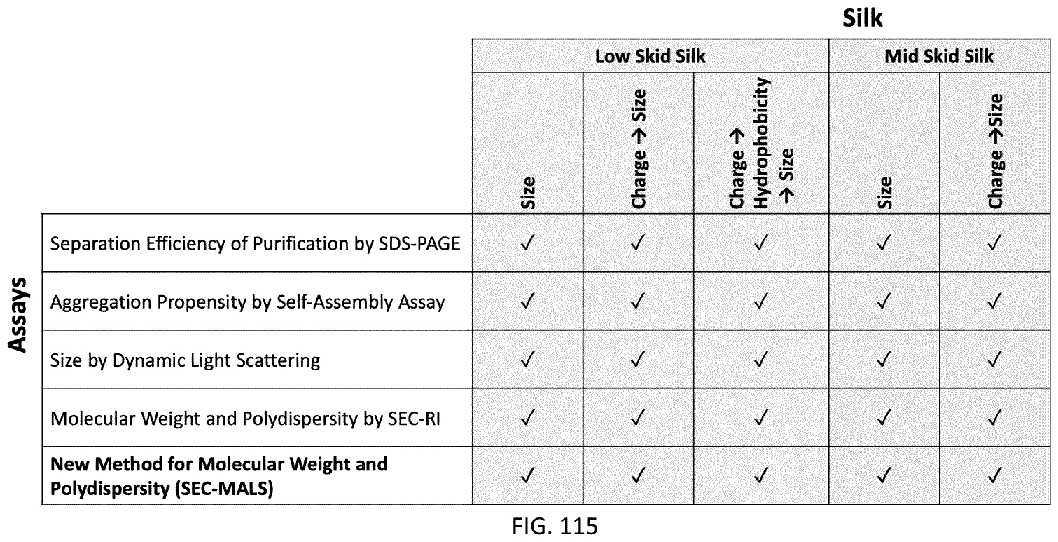

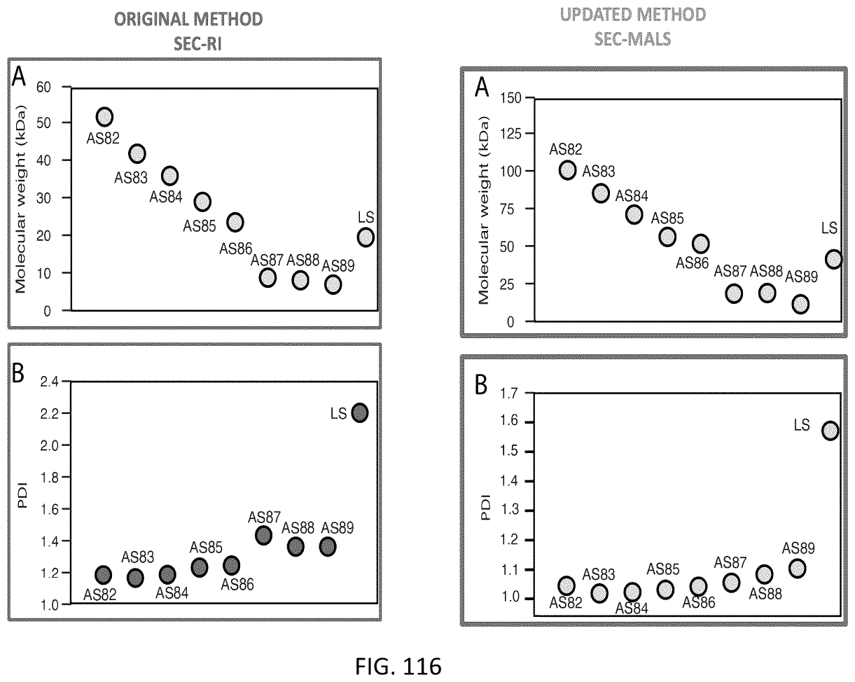

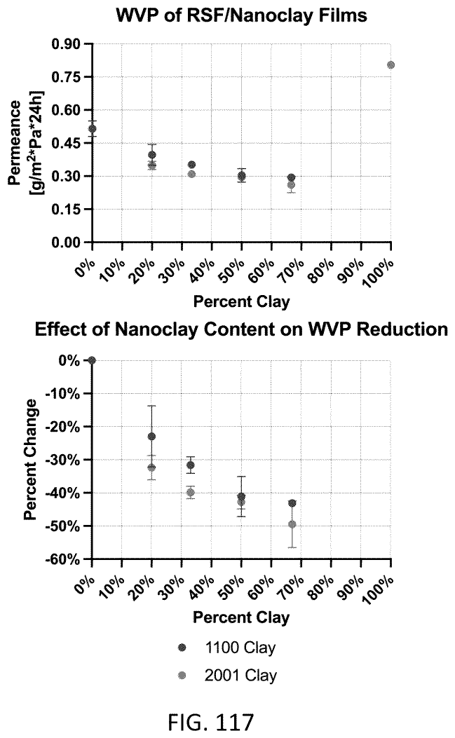

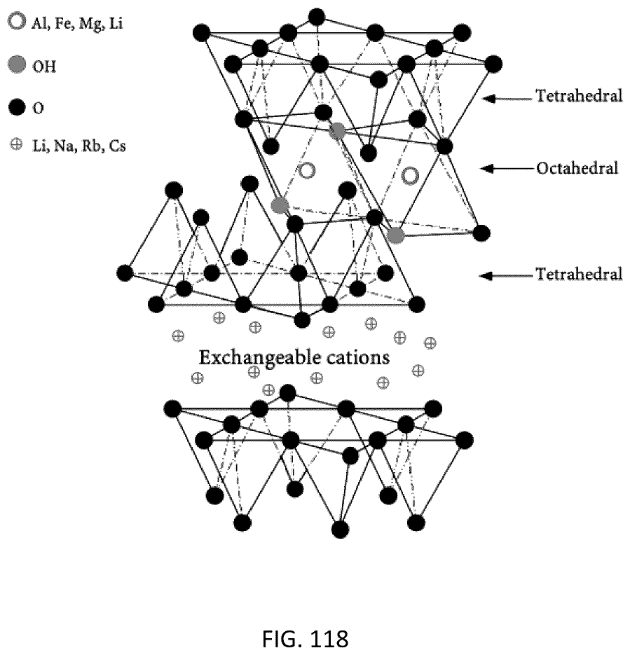

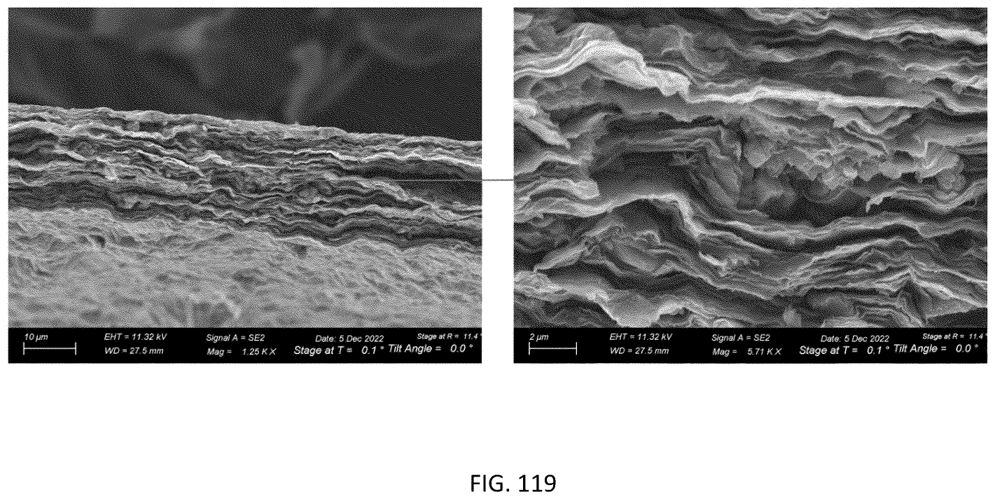

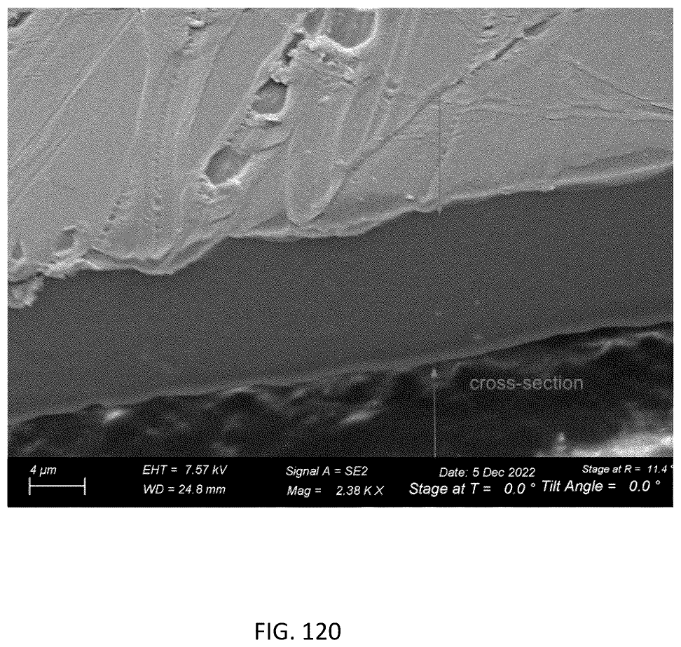

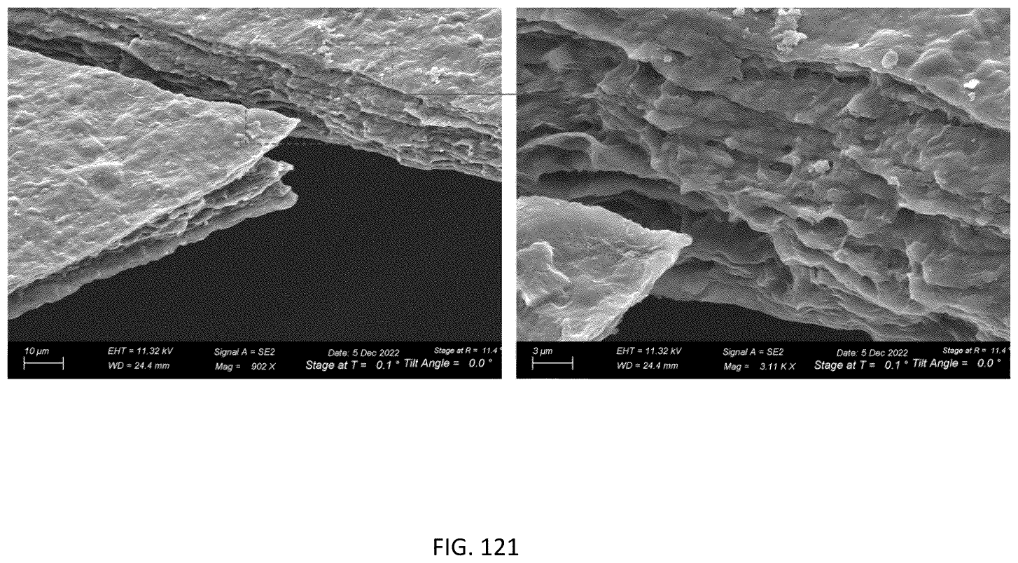

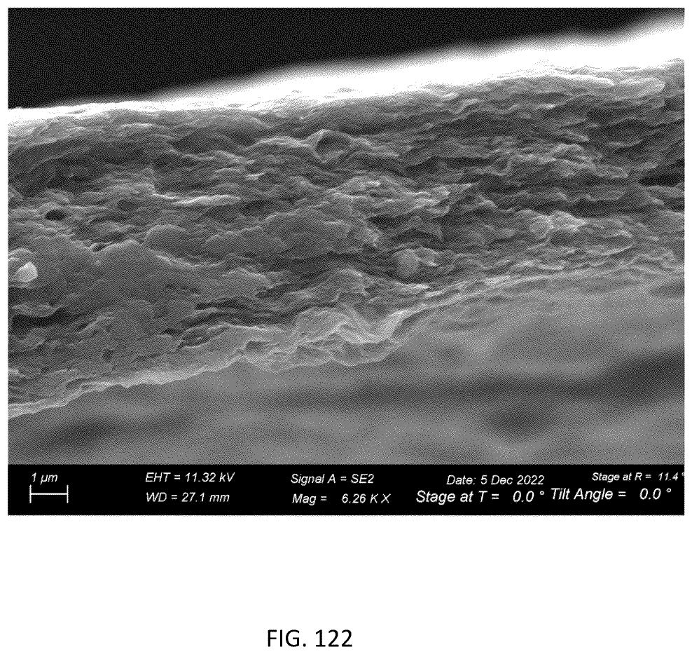

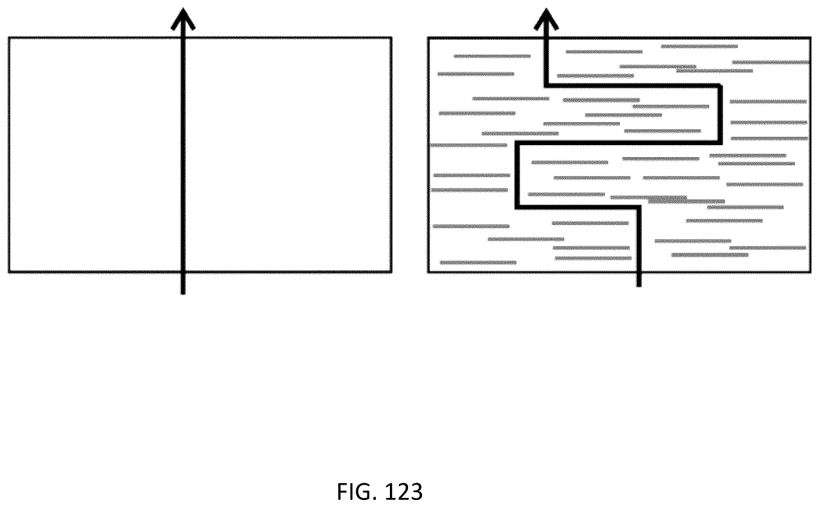

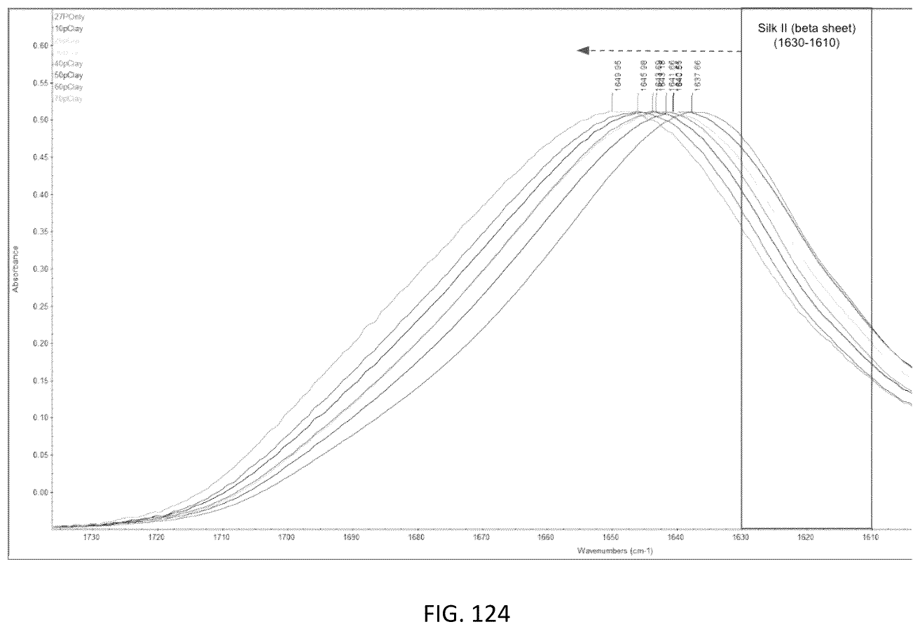

For 17120511 blue, the absorbency of the standard AS-320 coated fabric was around 3 seconds at T=5 and T=10. However, with HT-232H fixing agent pretreatment, the absorbency of the standard AS-320 coated fabric improved to around 1 second at T=5 and T=10. Same as 17120513, both AS-320 and the fixing agent contributed to the low absorption time measured. With only HT-232H fixing agent treatment, the fabric had an absorbency around 4 to 5 seconds at T=5 and T=10 (FIG. 95).

From this study, it was learned that pretreatment with HT-232H cationic fixing agent can result in an improved absorbency of AS-320 coated recycled polyester fabrics.

### Example 22: Barrier Redux Emulsion Formulation

In a large beaker, water (1216.80 g, 81.12%), propanediol (45.00 g, 3.00%), and allantoin (3.75 g, 0.25%) were measured out and mixed at medium speed until homogenous while heating to 75° C. In a separate beaker, Montanov L (15.00 g, 1.00%) (an alkylpolyglucoside emulsifier, including C14-22 Alcohols and C12-20 Alkyl Glucoside), and Caprylic Capric Triglycerides (CCT) oil (60.00 g, 4.00%; the triglycerides and esters prepared from fractionated vegetable oil sources and fatty acids from coconuts and palm kernel oils) were measured out and heated to 75° C. while stirring moderately with a magnetic stirrer.

Once both phases hit the target temperature, Ecogel (7.50 g, 0.50%; an emulsifying agent comprising lysolecithin, pullulan, sclerotium gum, and xanthan gum, specialchem.com) was dispersed into the water phase and homogenized for 2 minutes at 8000 rpm. The oil phase was then added, and the mixture homogenized for 10 minutes to form an emulsion. While the emulsion phase was cooling, water (75 g, 5.00%, sodium phytate (3.00 g, 0.20%) and aspen bark (22.50 g, 1.50%) were measured out into a separate beaker and stirred until homogenous. Once the emulsion had cooled to below 40° C., this preservative blend was added. The emulsion was further cooled to room temperature. At this time, Activated Silk 33B was added in the form of a 6% solution (49.95 g, 3.33%, equating to 2.00% 33B+1.33% water). pH was then adjusted to ˜5.5 with the addition of a 50% aqueous solution of citric acid (1.50 g, 0.10%). Once homogenous, stirring was stopped and the product was packaged into frosted glass dropper bottles.

### Example 23: Molecular Weight Analysis of Silk Protein by SEC-RI

This example defines the procedure for measuring the molecular weight of silk protein using size-exclusion chromatography with refractive index detection (SEC-RI). To create a calibration curve of Dextran Standards, with nominal molecular weights (Mw) of 5 kDa, 12 kDa, 25 kDa, 50 kDa, and 150 kDa, to determine the Molecular weight (Mw) and Polydispersity (PD) values of Evolved By Nature (EBN) processed silk protein.

Described herein is the procedure used to operate the high-performance liquid chromatography (HPLC) SEC-RI system, including the preparation of mobile phase and sample solutions, system startup, column equilibration, system shutdown, to create a calibration curve of Dextran standards using SEC data collection and analysis software; and to determine the molecular weight of silk protein and its derivatives. The values obtained from this process will contribute to protein characterization for research and development purposes and quality assurance. The molecular weight can further be used as a requirement for batch-release of all future production of silk solutions.

### Software:

Agilent Technologies OpenLAB CDS ChemStation Edition for LC & LC/MS Systems software (Rev C. 01.08 [210]), or equivalent
Cirrus SEC data collection and molecular weight analysis software, or equivalent

### Materials:

Silk solution
Reverse Osmosis (RO) or Deionized (DI) water
Sodium chloride (VWR, Part No. BDH9286 or equivalent)
Sodium phosphate dibasic heptahydrate (VWR, Part No. BDH9296 or equivalent)
Phosphoric acid (VWR, Part No. BDH153155E or equivalent)
Dextran molecular weight standard 5KDa (Sigma-Aldrich, Part No. 00269, or equivalent)
Dextran molecular weight standard 12KDa (Sigma-Aldrich, Part No. 00270, or equivalent)
Dextran molecular weight standard 25KDa (Sigma-Aldrich, Part No. 00271, or equivalent)
Dextran molecular weight standard 50KDa (Sigma-Aldrich, Part No. 00891, or equivalent)
Dextran molecular weight standard 150KDa (Sigma-Aldrich, Part No. 00893, or equivalent)
5% w/v aqueous sodium azide solution (VWR Chemicals)

### Equipment:

HP Z240 Workstation computer, or equivalent
HP EliteDisplay E190i LED monitor, or equivalent
HPLC system (Agilent 1260 Infinity II LC System, or equivalent)
Refractive Index Detector (Agilent G7162A RID, or equivalent)
PolySep GFC P-4000 LC Column, 300 mm×7.8 mm (Phenomenex, Part No. CHO-9229)
Mettler Toledo XP26 Delta Range micro analytical balance
pH Meter (Mettler Toledo FiveEasy F20 or Thermo Scientific Orion Star A221 or equivalent)
pH standards
Vacuum driven disposable filtration system, PES 0.2 um (Millipore Express)
Vacuum pump (Vacuubrand, Part No. ME 1)
Autosampler vials (clear glass, 2 mL) and vial caps
Autosampler vial tray
Syringe filter (PTFE membrane, 0.45 μm)
3 mL syringe
5 mL microcentrifuge tubes
Graduated cylinder
Glass beakers
Glass media bottles
a Magnetic stir bars
a Disposable weigh boats
a Disposable pipettes
Eppendorf pipets

### Procedure:

### Preparation of 1 L Mobile Phase (0.1M Sodium Chloride Solution in 12.5 mM Sodium Phosphate Buffer, pH 7).

Weigh 3.35 g of sodium phosphate heptahydrate and record the weight. Weigh 5.84 g of sodium chloride and record the weight. Transfer both solids to a beaker. Add 1000 mL of RO/DI water to the beaker and mix until solids are dissolved. Once dissolved adjust the pH of the solution to 7.0±0.2 with phosphoric acid. Filter the solution through a 0.2 μm PES filter into a clean glass media bottle.
Label the bottle ‘MW Mobile Phase, 0.1M NaCl+12.5 mM Na₂HPO₄’ and include the pH of the solution, the date prepared, notebook reference and the expiry of the solution. Store at room temperature for up to 1 week.
(Note: The volume of the solution can be varied as long as the concentration of sodium phosphate and sodium chloride remains as defined above.)

### System Startup and Column Equilibration.

1. Select the LC OpenLAB icon ‘HPLC (online)’ from the desktop to open the Agilent HPLC control software.
2. Load the pre-set method that will be used to run samples for silk molecular weight analysis. The method parameters that should be used for all silk molecular weight testing are listed in Table 1. If the same column and mobile phase that are currently installed are going to be used for this run, then proceed to step 7.2.15.
   File→Load→Method→SEC-RI_MRL Method.M
3. Update the program with the correct volume of mobile phase connected to the system. (Always input at least 0.20 L less volume for the Actual volume than the Total Volume). Under Actions, select and input ‘Prevent analysis if level falls below 0.20 liter’ and ‘Turn pump off if running out of solvent’.
   Right click Quat. Pump→Bottle Fillings
4. Manually open the purge valve located in the quaternary pump by turning the nozzle counterclockwise. The mobile phase will now flow directly to waste and not enter the column compartment.
5. Once the purge valve is open and the mobile phase is not flowing in the direction of the column compartment purge the mobile phase tubing at 5 mL/min until no bubbles are detected. Reduce the flow to 1 mL/min and close the valve.
6. Turn on both temperature controllers for the column compartment and refractive index detector. Do not turn on without flow going through the column.
   Right click Column Comp→Switch On
   Right click RID→Switch On

TABLE 51
SEC-RI_MRL Method parameters.

| Column: | PolySep GFC P-4000 (7.8 × 300 mm) | |
|---|---|---|
| Column | 25° | C. |
| Temperature: | | |
| Detector: | Refractive Index Detector | |
| Detector | 35° | C. |
| Temperature: | | |
| Injection Volume: | 25 | μL |
| Mobile Phase: | 0.1M Sodium Chloride 12.5 mM Sodium | |
| | phosphate buffer | |
| Flow Rate: | 1.0 | mL/min |
| Run Time: | 20.0 | min |

7. Open the RID purge valve to purge the reference cell with new mobile phase.
   Right click RID→Open Purge Valve
8. If not already displayed, add both the pressure and RID signals to the Online Plot window.
   Online Plot window→Change . . . →Under Available Signals select PMPIA, Pressure (bar) and RIDIA, RI Signal (nRIU)→Add
      Preparation of 1 mg/mL Dextran Molecular Weight Standard Solutions
1. Weigh 5 mg of each dextran standard into 5 separate 5 mL micro centrifuge tubes. Record the weight for each standard. Dissolve each standard in 5 mL of mobile phase. Let the solutions sit for at least 1 hour at room temperature to ensure homogeneity.
2. Slowly filter the standard solutions through a 0.45 μm syringe filters into HPLC vials and cap tightly.
   Preparation of 1 mg/mL Sample Solutions
1. Dilute the silk solution with mobile phase to 1 mg/mL in a 1.5 mL microcentrifuge tube. For a 6% silk solution, weigh approximately 83 mg of silk solution into a 5 mL microcentrifuge tube and add 5 mL of mobile phase. Invert the microcentrifuge tube 10 times to mix the solution.
2. Slowly filter the sample solution through a 0.45 μm syringe filter into a glass HPLC vial and close with an HPLC vial cap.
   (Note 1: Fast filtration may induce shearing in the sample)
   (Note 2: If there is limited amount of sample, the volume of sample solution can be scaled down as long as the final sample concentration remains at I mg/mL.)

### Running Dextran Standards and Sample Solutions.

1. Filter mobile phase into a HPLC vial to be used for blank runs.
2. Place each vial into the autosampler.
3. Edit the sequence table and list the order of samples that are going to be run. Input the sample location, name, and method. Always run standard solutions at the beginning of the run, after 12 injections, and at the end of the run. Make the last run of the sequence a blank run using the FlowDecrease method. Sequence Window→right click blue box→Sequence Table . . .
4. Close the RID purge valve. Right click RID→Close Purge Valve
5. Once all of the boxes under the Instrument Control tab turn green, the system is ready to run the sample sequence. Select Start Sequence to begin the run. If a message appears stating, ‘MethodSEC-RI_MRL Method.M has changed. Save current changes?’, select No to ensure the method saved in the software does not change.
6. Once the run is complete, the mobile phase will continue to flow through the system at the flow rate set for the wash method, 0.05 mL/min, as it is the last run of the sequence. If the experiment is done, then the system can be shutdown according to the procedure noted in 7.6.

### System Shutdown

1. If the system does not need prolonged storage, the flow rate can be set to 0 ml/min.
2. For prolonged storage, rinse and store the system and column in 0.05% sodium azide in RO/DI water. Rinse the system with filtered RO/DI water prior to flushing with sodium azide solution.
   To make a 1 L 0.05% sodium azide solution in water, add 10 mL of 5% sodium azide solution to 990 mL of RO/DI water.
3. Once the column and system has been rinsed with sodium azide at 1 mL/min for at least 1 hour, the flow rate can be placed to 0 mL/min and/or the column can be disconnected.

### Calibration Curve Procedure:

1. Select the LC OpenLAB icon ‘HPLC (offline)’ from the desktop to open and view collected data.
2. Open the Cirrus analysis software.
   Data Analysis→select the sample set to be analyzed→Cirrus→GPC Analysis
3. Create a calibration method.
   Make a new method to be used for creating a calibration curve and analyzing a set of samples.
      Method Browser→right click on the table→New Method . . .
   Under the General tab, input a detailed name of the method and include in the comments the mobile phase, column information, and flow rate. Select Do Data Processing and input 60 secs for the Time between chromatograms.
   Under the Column Calibration tab, next to Generate Using select Narrow Standard. Input Apply a Polynomial curve, of order 3. For Calibrant Automation, select Manual Processing and check Detect Peaks Automatically.
   Under the Sample Analysis tab, for Automation select Manual Processing and check Detect Peaks Automatically. Under Calculation check Perform GPC Calculations and select Area Integration for the Calculation Method using 1 data point in 1. Check Calculate MW Ranges and select the Details option.
   A new window will appear containing a MW Ranges table. Input the M_{w} value listed on the COA of the Dextran Standard with the highest Mw under the High MW Limit column and input the M_{W} value listed in the COA with the lowest Mw value under the Low MW Limit column.
   Under the Peak Detection tab, input the values listed in Table 52 into each column of the Peak Detection table. Under Baseline Assignment, check Apply Horizontal Baseline and Start of First Peak.

TABLE 52
Peak Detection table input values.

| Type | System detects |
|---|---|
| Start [mins] | 5.00 |
| End [mins] | 10.00 |
| Slope | 0.1000 |
| Separation Width [secs] | 8 |
| Noise Tolerance | 5.0000 |
| Negative Peaks | Unchecked box |
| Min. Height [mV] | 0.0000 |
| Min. Area [mV · s] | 0.0000 |
| Valley-to-Valley | Uncheck box |
| Skim Ratio | 8.0000 |

Leave the tables shown under the Reporting and User Programs tabs blank. Select OK to exit out of the window. The method should now appear in the Methods Browser list and have a symbol indicating that the method has not been calibrated yet and cannot be used.
4. Create a calibration curve using Dextran Standards.
   Select Analysis Runlist and clear any previous samples that are listed in the runlist table.
      Select the table→Runlist→Clear Runlist
   The Open the folder that contains the Dextran standard runs that will be used for the calibration curve.
   Add the RI signal for each Dextran standard (5 kDa, 12 kDa, 25 kDa, 50 kDa, and 150 kDa) to the runlist table by double clicking on the RI signal file.
   For each standard in the runlist table, input Sample Type=Narrow Standard and Cal. Version=Create. Add the method that was previously created to be used for the calibration curve. Leave all other columns as default.
      Double click Method box next to first standard→Choose Method→Fill Down
   Add the first standard to the Analysis window by selecting the Next Analysis icon. The text of the first standard on the list will turn from blue to green when selected.
   View the chromatogram under the Analysis tab and select Peak Summary. Delete all of the auto-labeled peaks.
      Highlight each peak in the Analysis window→hit Delete on keyboard
   Visually analyze the chromatogram of each standard. The graph should show a single narrow curved peak with a flat baseline. Make sure impurities are limited and do not significantly interfere with the main peak. If the peak is broad and/or the baseline is uneven, there is something wrong with the column or system that needs to be addressed before the samples can be analyzed.
   Identify and label the main peak of the chromatogram.
      Analysis→Detect Peak Max→click on the top of the peak on the chromatogram.
   Double check the sample name by selecting the Sample Summary tab. Return to the Peak Summary tab and input the Peak Name and record the Max. RT listed in the table.
   Enter the Mₚ value listed on the COA for the Dextran Standard of that chromatogram in the Peak MW column. The rest of the columns will autofill in from internal calculations of the software.
   Add the value to the calibration curve by select the Add to Calibration icon. An error may appear indicating Insufficient Data Points; select OK to continue. The error will not appear as more points are added. The next sample chromatogram in the runlist will automatically appear with a point on the graph indicating the start of the calibration curve. If the next chromatogram does not appear, select the Next Analysis icon for it to show.
   Repeat steps 7.4.6-7.4.10 until all of the Dextran standards have been added to the calibration curve.
   To Check and make sure that the method has been calibrated, select the MethodBrowser tab. The calibration status should now show an icon indicating the method has been calibrated.
   To analyze the calibration curve details, select the Calibration Browser tab. Make sure the Coefficient of Determination value is at least 0.99.
      Select the method→Right click on the table→Show Calibration Details

### Sample Analysis Procedure:

1. After creating the calibration curve, add sample runs to the analysis window.
   Add the sample RI signal to the runlist table by double clicking on the RI signal file. Analyze all standard samples first, then silk lot samples.
   In the runlist table, make sure the sample type is listed as Unknown and add the calibration method that was previously created to be used for analysis. Leave all other columns as default.
      Double click Method box next to first standard→Choose Method→Fill Down or OK
   Add the first standard to the Analysis window by selecting the Next Analysis icon. The text of the first standard on the list will turn from blue to green when selected.
   View the chromatogram under the Analysis tab and select Peak Summary. Delete all of the auto-labeled peaks.
   Highlight each peak listed in the Peak Summary table→hit Delete on keyboard
   Visually analyze the chromatogram and report any additional peak formations that could be signs of protein aggregation or impurities, such as excess lithium bromide.
2. Integrate the RI peaks for each sample solution and determine the Mw and PD values through the software.
   Identify the main positive peak of the silk protein solution found within the retention time of 5-10 min. A majority of the peak should fall within range of calibration curve between the high and low limits.
   Integrate the peak from left to right, aligning the baseline at both ends of the peak. Do not include the solvent mis-match peak in the integration. Zoom into the peak if needed. Afterwards, a red line will appear at the baseline and the peak will be labeled and added to the Peak Summary table.
      Analysis→Zoom→drag curser along the chromatogram to box area of interest and zoom-in
      Analysis→Detect Peaks and Baseline
   Select Calculate Results to fill in the rest of the table.
   Record the following values on the batch record: Mw and PD.

### Example 24: Body Wash Clinical Study

A Body Wash has been developed containing Activated Silk. A clinical study including bioinstrumentation, expert grading and a self-perception questionnaire was completed using Evolved by Nature Body Wash.

### Test Materials:

### Body Wash Lot #: STI-146-52-01

### Study Protocol

Objective: The objective of this study is to evaluate the efficacy of Evolved by Nature Body Wash.
Subjective Demographics: An adequate number of subjects were enrolled to ensure that a minimum of 30 completed the study.

Inclusion criteria included:
Healthy male and female subjects aged 18+.
Subjects must have a minimum of 1 active eczema lesion.
50% subjects must have self-perceived sensitive skin.
Subject has completed a 7 day wash out.
Subject has signed a written Informed Consent.
Subject has signed a photography release form

Methods: Subjects were given the test articles to use as instructed at home for 7 days. At baseline, immediately and after 7 days subjects underwent expert grading assessments, Corneometer and TEWL assessment. Self-perception questionnaires were completed immediately post application and following one week of use.

### Requirements/Instructions:

| | | |
|---|---|---|
| 1 | Alternate Names | EBN Body Wash |
| 2 | Manufacturing | Procedure: MFR-CAST-051 |
| | Instructions | Batch Record Form: MFR-CAST-051-F01 |
| 3 | Safety Precautions | Handle in accordance with good industrial |
| | | hygiene and safety practices. |
| 4 | Handling and Storage | Store at room temperature in an airtight |
| | | container. Protect from light. |
| 5 | Certificate/Documents | Safety Data Sheet, Certificate of Analysis |
| 6 | Shelf Life from Date of | 18 months |
| | Manufacture | |

### Material Specifications:

Product Name: Evolved by Nature Body Wash.
Product Size: 12 oz
Material Characteristics:

TABLE 53
Material Specifications

| Characteristic | Specification | Test Method |
|---|---|---|
| Color and | White opaque cream | Visual |
| Appearance | | |
| Odor | Unscented/characteristic | Smell |
| pH | 5.0-5.5 | EBN-EOP-PH-001 |
| Viscosity | 20,000 cps-35,000 cps | Viscometer |
| | | (Brookfield DVE) |
| | | Spindle #5, 10 rpm |
| | | 21 ± 5° C. |
| Microbial | ≤10² CFU/mL Total Mesophilic | USP <61> |
| enumeration | Microorganisms: maximum | |
| | acceptable count = 200 | |

TABLE 54
Formulation: STI-LAB-146-52

| Ingredient | Name | Part# | Wt (%) | |
|---|---|---|---|---|
| RO/DI Water | RO/DI Water | | 69.47% | (±1.0%) |
| DL-Panthenol | DL-Panthenol | RM-0023 | 0.9% | (−0.01%) |
| Geogard Ultra | Geogard ultra | RM-0069 | 1% | (±0.05%) |
| Jaguar C 13S SGI | Cationic Guar Gum | RM-0073 | 0.5% | (±0.01%) |
| TEGO ® White50 MB | TEGO ® White 50 MB | RM-0108 | 0.85% | (±0.01%) |
| Geropon 3G CS | Sodium Cocoyl Glycinate | RM-0080 | 15% | (±0.05%) |
| Plantaren 1200 N UP | Lauryl Glucoside | RM-0072 | 10% | (±0.05%) |
| Oramix CG 110 | Caprylyl/Capryl Glucoside | RM-0066 | 0.9% | (±0.002%) |
| Activated Silk (mid mw, 6%) | AS-5003-000 | AS-5003-000 | 0.83% | (±0.02%) |
| Citric Acid (50% Solution) | Citric Acid | RM-0018 | 0.55% | (qs) |

TABLE 55
Formulation with Split preservative:

| Ingredient | Internal Name | Internal Part# | Wt (%) | |
|---|---|---|---|---|
| RO/DI Water | RO/DI Water | | 69.47 | (±1.0%) |
| DL-Panthenol | DL-Panthenol | RM-0023 | 0.9% | (−0.01%) |
| Gluconolactone | Gluconolactone | RM-0106 | 0.75% | (±0.05%) |
| Sodium Benzoate | Sodium Benzoate | RM-0105 | 0.25% | (±0.05%) |
| Jaguar C 13S SGI | Cationic Guar Gum | RM-0073 | 0.5% | (±0.01%) |
| TEGO ® White 50 MB | TEGO ® White 50 MB | RM-0108 | 0.85% | (±0.01%) |
| Geropon 3G CS | Sodium Cocoyl Glycinate | RM-0080 | 15% | (±0.05%) |
| Plantaren 1200 N UP | Lauryl Glucoside | RM-0072 | 10% | (±0.05%) |
| Oramix CG 110 | Caprylyl/Capryl Glucoside | RM-0066 | 0.9% | (±0.002%) |
| Activated Silk (mid mw, 6%) | AS-5003-000 | AS-5003-000 | 0.83% | (±0.02%) |
| Citric Acid (50% Solution) | Citric Acid | RM-0018 | 0.55% | (qs) |

### Results

The results can be seen in the below tables. The following categories showed a statistically significant improvement after 7 days:
Improvement of Skin Hydration
Improvement of Barrier Function
Reduction of appearance of eczema
Improvement of redness

### Self-Perception Questionnaire:

The Self-perception Questionnaires showed favorable results.

TABLE 56
Self-perception Questionnaires

| | | Not Top Box | | Top Box | | p- |
|---|---|---|---|---|---|---|
| Questions | Total | N | % | N | % | value |
| 1. The product gives a soothing and moisturizing cleanse. | 30 | 3 | 10.00% | 27 | 90.00% | <0.01* |
| 2. The product instantly provides relief from dryness and itchiness. | 30 | 10 | 33.33% | 20 | 66.67% | 0.10 |
| 3. The product helps alleviate dryness and itchiness long after | 30 | 12 | 40.00% | 18 | 60.00% | 0.36 |
| washing. | | | | | | |
| 4. The product gently cleanses to soothe very dry skin. | 30 | 5 | 16.67% | 25 | 83.33% | <0.01* |
| 5. The product helps soothe and calm irritation. | 30 | 6 | 20.00% | 24 | 80.00% | <0.01* |
| 6. The product helps soothe and alleviate itchy skin. | 30 | 9 | 30.00% | 21 | 70.00% | 0.04* |
| 7. The product helps soothe and reduce redness and discomfort of | 30 | 7 | 23.33% | 23 | 76.67% | <0.01* |
| skin. | | | | | | |
| 8. The product helps alleviate rough, dry areas of skin. | 30 | 7 | 23.33% | 23 | 76.67% | <0.01* |
| 9. The product helps protect skin from drying effects of water. | 30 | 8 | 26.67% | 22 | 73.33% | 0.02* |
| 10. The product increases comfort while showering. | 30 | 6 | 20.00% | 24 | 80.00% | <0.01* |
| 11. Skin feels instantly soothed and moisturized after using this | 30 | 7 | 23.33% | 23 | 76.67% | <0.01* |
| product. | | | | | | |
| 12. Skin feels comfortable and nourished after using this product. | 30 | 7 | 23.33% | 23 | 76.67% | <0.01* |
| 13. Skin looks and feels softer and more supple after using this | 30 | 8 | 26.67% | 22 | 73.33% | 0.02* |
| product. | | | | | | |
| 14. Skin feels smoother after using this product. | 30 | 7 | 23.33% | 23 | 76.67% | <0.01* |
| 15. Skin feels more protected against irritants. | 30 | 9 | 30.00% | 21 | 70.00% | 0.04* |
| 16. My skin looks and feels healthier after using this product. | 30 | 5 | 16.67% | 25 | 83.33% | <0.01* |
| 17. The product is ideal for skin with eczema. | 30 | 5 | 16.67% | 25 | 83.33% | <0.01* |
| 18. The product is gentle enough to use daily and does not leave a | 30 | 6 | 20.00% | 24 | 80.00% | <0.01* |
| residue on skin after rinsing. | | | | | | |
| 19. The product provides instant relief eczema flare-up. | 30 | 9 | 30.00% | 21 | 70.00% | 0.04* |
| 20. The product alleviates the skin conditions of eczema and reduces | 30 | 7 | 23.33% | 23 | 76.67% | <0.01* |
| the appearance skin redness, dryness and flakiness. | | | | | | |

*Significant

TABLE 57
Self-perception Questionnaires

| | | Not Top Box | | Top Box | | p- |
|---|---|---|---|---|---|---|
| Questions | Total | N | % | N | % | value |
| 1. The product gives a soothing and moisturizing cleanse. | 30 | 2 | 6.67% | 28 | 93.33% | <0.01* |
| 2. The product instantly provides relief from dryness and itchiness. | 30 | 6 | 20.00% | 24 | 80.00% | <0.01* |
| 3. The product helps alleviate dryness and itchiness long after | 30 | 3 | 10.00% | 27 | 90.00% | <0.01* |
| washing. | | | | | | |
| 4. The product gently cleanses to soothe very dry skin. | 30 | 5 | 16.67% | 25 | 83.33% | <0.01* |
| 5. The product helps soothe and calm irritation. | 30 | 3 | 10.00% | 27 | 90.00% | <0.01* |
| 6. The product helps soothe and alleviate itchy skin. | 30 | 2 | 6.67% | 28 | 93.33% | <0.01* |
| 7. The product helps soothe and reduce redness and discomfort of | 30 | 5 | 16.67% | 25 | 83.33% | <0.01* |
| skin. | | | | | | |
| 8. The product helps alleviate rough, dry areas of skin. | 30 | 3 | 10.00% | 27 | 90.00% | <0.01* |
| 9. The product helps protect skin from drying effects of water. | 30 | 0 | 0.00% | 30 | 100.00% | <0.01* |
| 10. The product increases comfort while showering. | 30 | 1 | 3.33% | 29 | 96.67% | <0.01* |
| 11. Skin feels instantly soothed and moisturized after using this | 30 | 4 | 13.33% | 26 | 86.67% | <0.01* |
| product. | | | | | | |
| 12. Skin feels comfortable and nourished after using this product. | 30 | 4 | 13.33% | 26 | 86.67% | <0.01* |
| 13. Skin looks and feels softer and more supple after using this | 30 | 3 | 10.00% | 27 | 90.00% | <0.01* |
| product. | | | | | | |
| 14. Skin feels smoother after using this product. | 30 | 4 | 13.33% | 26 | 86.67% | <0.01* |
| 15. Skin feels more protected against irritants. | 30 | 3 | 10.00% | 27 | 90.00% | <0.01* |
| 16. My skin looks and feels healthier after using this product. | 30 | 4 | 13.33% | 26 | 86.67% | <0.01* |
| 17. The product is ideal for skin with eczema. | 30 | 3 | 10.00% | 27 | 90.00% | <0.01* |
| 18. The product is gentle enough to use daily and does not leave a | 30 | 3 | 10.00% | 27 | 90.00% | <0.01* |
| residue on skin after rinsing. | | | | | | |
| 19. The product provides instant relief eczema flare-up. | 30 | 3 | 10.00% | 27 | 90.00% | <0.01* |
| 20. The product alleviates the skin conditions of eczema and reduces | 30 | 4 | 13.33% | 26 | 86.67% | <0.01* |
| the appearance skin redness, dryness and flakiness. | | | | | | |

*Significant.

### Conclusions

At the conclusion of this study, the Body Wash that was tested validates below
Clinically Tested
Clinically Proven
Dermatologist Tested
Improves redness
Reduced the appearance of eczema
Improves skin hydration
Improves barrier function

### Example 25: Body Wash Consumer Report

Scope: Evolved by Nature has developed a new body wash product. A consumer study including a self-perception questionnaire was completed while using the body wash for a total of 7 days.

### Test Materials:

### Body Wash; Lot #: STI-146-52-01

### Study Protocol

Objective: The objective of this study is to evaluate the efficacy of Evolved by Nature Body Wash to substantiate claims.
Subjective Demographics: An adequate number of subjects were enrolled to ensure that a minimum of 30 completed the study.
Demographics are summarized in the below table. Inclusion criteria included:
   Ages 18+ with self-perceived eczema prone skin

TABLE 58
Study Demographics

| Age | | |
|---|---|---|
| | Mean | 41.22 |
| | Std | 14.01 |
| | Median | 42.5 |
| | Range | 18-60 |
| Race | | |
| | Caucasian | 17 |
| | Asian | 9 |
| | Bi-racial | 2 |
| | Black | 4 |
| Sex | | |
| | Female | 23 |
| | Male | 9 |

Methods: Subjects were given the test articles to use as instructed at home for 7 days. Subjects were required to discontinue using any moisturization products including oils, lotions, and creams. Subjects returned on study day 0 and were given the test article to use at home for the remainder of the study. Subjects then returned to the testing facility on study day 7. Subjects completed self-perception questionnaires instantly post use and following 7 days.

### Results

The results can be seen in the below tables.

TABLE 59
Self Perception Questionnaire Instant

| | | Top Box | | Not Top Box | | p- |
|---|---|---|---|---|---|---|
| Questions | Total | N | % | N | % | value |
| The product gives gentle and effective | 32 | 32 | 100.00% | 0 | 0.00% | <0.01* |
| cleansing without irritating skin. | | | | | | |
| The product gives a soothing and moisturizing | 32 | 32 | 100.00% | 0 | 0.00% | <0.01* |
| cleanse. | | | | | | |
| The product instantly provides relief from | 32 | 31 | 96.88% | 1 | 3.12% | <0.01 |
| dryness and itchiness. | | | | | | |
| The product helps alleviate dryness and | 32 | 17 | 53.12% | 15 | 46.88% | 0.72 |
| itchiness long after washing. | | | | | | |
| The product gently cleanses to soothe very dry | 32 | 27 | 84.38% | 5 | 15.62% | <0.01* |
| skin. | | | | | | |
| The product helps sooth and calm irritation. | 32 | 30 | 93.75% | 2 | 6.25% | <0.01* |
| The product helps sooth and alleviate itchy | 32 | 30 | 93.75% | 2 | 6.25% | <0.01* |
| skin. | | | | | | |
| The product helps soothe and reduce redness | 32 | 20 | 62.50% | 12 | 37.50% | 0.16 |
| and discomfort on skin. | | | | | | |
| The product helps alleviate rough, dry areas of | 32 | 28 | 87.50% | 4 | 12.50% | <0.01* |
| skin. | | | | | | |
| The product helps protect skin from crying | 32 | 32 | 100.00% | 0 | 30.00% | <0.01* |
| effects of water. | | | | | | |
| The product increases comfort while | 32 | 31 | 96.88% | 1 | 3.12% | <0.01* |
| showering. | | | | | | |
| Skin feels instantly soothed and moisturized | 32 | 32 | 100.00% | 0 | 0.00% | <0.01* |
| after using this product. | | | | | | |
| Skin feels comfortable and nourished after | 32 | 32 | 100.00% | 0 | 0.00% | <0.01* |
| using this product. | | | | | | |
| Skin looks and feels softer, more supple after | 32 | 31 | 96.88% | 1 | 3.12% | <0.01* |
| using this product. | | | | | | |
| Skin feels smoother after using this product. | 32 | 31 | 96.88% | 1 | 3.12% | <0.01* |
| Skin feels more protected against irritants. | 32 | 18 | 56.25% | 14 | 43.75% | 0.48 |
| Skin looks and feels healthier after using this | 32 | 27 | 84.38% | 5 | 15.62% | <0.01* |
| product. | | | | | | |
| The product is ideal for sensitive eczema-prone | 32 | 24 | 75.00% | 8 | 25.00% | <0.01* |
| skin. | | | | | | |
| The product is gentle enough to use daily and | 32 | 17 | 53.12% | 15 | 46.88% | 0.72 |
| does not leave residue on skin after rinsing. | | | | | | |

*Null hypothesis rejected.

TABLE 60
Self Perception Questionnaire- 7 day

| | | Top Box | | Not Top Box | | p- |
|---|---|---|---|---|---|---|
| Questions | Total | N | % | N | | value |
| The product gives gentle and effective | 32 | 27 | 84.38% | 5 | 15.62% | <0.01* |
| cleansing without irritating skin. | | | | | | |
| The product gives a soothing and moisturizing | 32 | 27 | 84.38% | 5 | 15.62% | <0.01* |
| cleanse. | | | | | | |
| The product instantly provides relief from | 32 | 27 | 84.38% | 5 | 15.62% | <0.01* |
| dryness and itchiness. | | | | | | |
| The product helps alleviate dryness and | 32 | 26 | 81.25% | 6 | 18.75% | <0.01* |
| itchiness long after washing. | | | | | | |
| The product gently cleanses to soothe very dry | 32 | 23 | 71.88% | 9 | 28.12% | <0.01* |
| skin. | | | | | | |
| The product helps sooth and calm irritation. | 32 | 26 | 81.25% | 6 | 18.75% | <0.01* |
| The product helps sooth and alleviate itchy | 32 | 27 | 84.38% | 5 | 15.62% | <0.01* |
| skin. | | | | | | |
| The product helps soothe and reduce redness | 32 | 25 | 78.12% | 7 | 21.88% | <0.01* |
| and discomfort on skin. | | | | | | |
| The product helps alleviate rough, dry areas of | 32 | 27 | 84.38% | 5 | 15.62% | <0.01* |
| skin. | | | | | | |
| The product helps protect skin from drying | 32 | 27 | 84.38% | 5 | 15.62% | <0.01* |
| effects of water. | | | | | | |
| The product increases comfort while | 32 | 27 | 84.38% | 5 | 15.62% | <0.01* |
| showering. | | | | | | |
| Skin feels instantly soothed and moisturized | 32 | 27 | 84.38% | 5 | 15.62% | <0.01* |
| after using this product. | | | | | | |
| Skin feels comfortable and nourished after | 32 | 27 | 84.38% | 5 | 15.62% | <0.01* |
| using this product. | | | | | | |
| Skin looks and feels softer, more supple after | 32 | 27 | 84.38% | 5 | 15.62% | <0.01* |
| using this product. | | | | | | |
| Skin feels smoother after using this product. | 32 | 27 | 84.38% | 5 | 15.62% | <0.01* |
| Skin feels more protected against initants. | 32 | 26 | 81.25% | 6 | 18.75% | <0.01* |
| Skin looks and feels healthier after using this | 32 | 26 | 81.25% | 6 | 18.75% | <0.01* |
| product. | | | | | | |
| The product is ideal for sensitive eczema-prone | 32 | 26 | 81.25% | 6 | 18.75% | <0.01* |
| skin. | | | | | | |
| The product is gentle enough to use daily and | 32 | 26 | 81.25% | 6 | 18.75% | <0.01* |
| does not leave residue on skin after rinsing. | | | | | | |

*Null hypothesis rejected.

### Example 26: Collagen Redux Serum EpiOcular

Purpose: The purpose of this study is to evaluate Collagen Redux Serum for irritancy potential utilizing the MatTek Corporation EpiOcular in vitro toxicity testing system.

Test Materials: Collagen Redux Serum (Lot #192-37)

Study Protocol: A 20% dilution was made with the sample and was added to Millicells with the EpiOcular samples. The well plates underwent a series of incubation periods, washing periods and extraction periods before the sample's absorbance was read on a plate reader. Using the percent viabilities determined from the absorbance, calculations were performed to obtain a Draize score, which corresponds to an irritancy classification.

Results: Collagen Redux Serum at 20% scored a “0” on the Draize Ocular Irritation scale and is classified as “non-irritating”.

TABLE 61
Results:

| Article | | Percent | Percent |
|---|---|---|---|
| (% & Exposure) | System | Viability | Inhibition |
| Collagen Redux Scrum; | | | |
| Lot umber: 192-37 | | | |
| (20%-4 hrs.) | EpiOcular | 107 | −7 |
| (20%-I hr.) | EpiOcular | 102 | −2 |
| (20%-20 mins.) | EpiOcular | 106 | −6 |
| Triton X-100 | | | |
| (0.3%-I hr.) | EpiOcular | 17 | 83 |
| (0.3%-20 min.) | EpiOcular | 67 | 33 |
| (0.3%-5 mins.) | EpiOcular | 92 | 8 |

Using a semi-log scale, the percent viabilities for the articles were plotted on the linear y axis versus the dosing time on the log x axis. The average OD570 for the negative control in the second interval was used in the calculation. By interpolation, the time at which the percent viability would be 50% was determined (ET-50), if possible. As a general guideline (provided by MatTek) the following equation can be used to estimate the rabbit Draize eye score:

$$Draize = - 4.74 + 101.7 / ( ET - S 0 ) 0.5$$

Based on the literature, the ocular irritancy estimated potential has been categorized by MatTek into the following groups based on the Draize score:

| Draize | Irritancy | | EpiOcular |
|---|---|---|---|
| Score | Classification | Example | ET-50 (min) |
| 0-15 | Non-irritating, | PEG-75 Lanolin, Tween 20 | >256-26.5 |
| | Minimal | | |
| 15.1-25 | Mild | 3% Sodium Dodecyl Sulfate | <26.5-11.7 |
| 25.1-50 | Moderate | 5% Triton X-100 | <117-3.45 |
| 50.1-110 | Severe, Extreme | 50% Benzalkonium Chloride | 3.45 |

### Discussion

Under the conditions of this test, the Collagen Redux Serum; test article, at 20%, elicited in vitro results which indicate that its ET-50 is greater than 256 minutes. Therefore, at 100%, the test article' estimated Draize ocular irritation core is approximately 0 with a “non-irritating” irritancy classification. The Triton X-100 reference/positive control article elicited in vitro results which place it ET-50 at 29.2 minutes. Therefore, the reference article's estimated Draize ocular irritation score is 14.1 with a “minimally irritating” irritancy classification.

### Conclusion

Under the conditions of this test, the result indicate that collagen Redux serum has a “non-irritating” irritancy classification. The test materials can be considered as safe for use under the conditions of the study.

### Example 27: Study to Evaluate the Potential of One Test Article to Produce a Phototoxic and/or Photoallergic Response

Study design: single-blind within-subject randomized study.
Test Article: Activated Silk 27P (in carrier serum) (TOX182-76/SEN182-79)
Negative control—0.9% Sodium Chloride (NaCl -Phototoxicity only)

Dose regime: Photoallergy portion—6 applications of the test article during the induction phase of the study, and 1 application during the challenge phase.
Phototoxicity portion—1 application of the test article.
Duration of study: 6 weeks

Number of subjects: 29 subjects completed the Photoallergy, and 12 subjects completed the Phototoxicity portion of the study, according to Sponsor instructions.
Type of subjects: Healthy male and female volunteers, aged 18 or older.

### Method: Photoallergy—Induction Phase

A designated area on each subject's back was irradiated on Day 1 and scored the following day to confirm each subject's historical MED.

Duplicate test sites on the back (each approximately 4 cm2) were identified. The test material was applied to designated patches and affixed to both sites on the back. One site was designated for irradiation, and the other was used as a treated, non-irradiated control. Approximately 24 hours later, the patches were removed, and the test sites were evaluated according to the dermal scoring scale. Following grading, one test site was irradiated with two times the subject's MED. The remaining site served as the non-irradiated control.

This procedure was carried out twice weekly for a total of six patch applications and post-irradiation readings. Irradiated areas within the treated sites and the entire treated area of the control sites (treated/non-irradiated) were examined approximately 24 hours following irradiation of the test sites and graded.

### Photoallergy—Challenge Phase

Approximately 10 to 21 days following the last application, duplicate patches were applied to sites previously unexposed to the test material. Approximately 24 hours later, the patches were removed. One test site was irradiated with a non-erythrogenic dose of UVA radiation equivalent to approximately 10 J/cm² while the second test site served as a test material treated, non-irradiated control site and a third untreated test site was also irradiated (control site). The challenge sites were graded at 24-, 48-, and 72-hours following irradiation according to the grading scale.

### Phototoxicity

Twelve (12) subjects were selected for the evaluation of phototoxic potential. These subjects had the test material applied under additional patches during the Photoallergy Induction Phase. Approximately 24 hours after application, the patches were removed, and the test sites were evaluated. The sites were irradiated with long wave (UVA 320-400 nm) radiation. An adjacent, untreated site (0.9% NaCl) was also irradiated (control site). Treated and control sites were examined at 24-, 48-, and 72-hours post-irradiation and graded.

### Inclusion Criteria

a. Subject is a healthy male or female between 18 and 70 years of age.
b. Subject has a Fitzpatrick Skin Type I-III, based on the first 30 to 45 minutes of sun exposure after a winter season of no sun exposure according to the following criteria:
   I—Always burns easily; never tans (sensitive)
   II—Always burns easily; tans minimally (sensitive)
   III—Burns moderately; tans gradually (normal)
c. Subject agrees not to introduce any new cosmetic or toiletry products during the study.
d. Subject agrees to refrain from getting patches wet and from scrubbing or washing the test area with soap or applying powders, lotions, or personal care products to the area during the course of the study.
e. Subject is dependable and able to follow directions as outlined in the protocol and anticipates being available for all study visits.
f. Subject is willing to participate in all study evaluations.
g. Completed written informed consent.

### Prohibitions and Restrictions

a. Discontinuation of aspirin or non-steroidal anti-inflammatory medication for the duration of the study.
b. Discontinuation of sun bed or sun lamp use, and avoidance of exposure of the test sites to natural sunlight for the duration of the study.

### Method

TABLE 62
Test Articles

| | Test Article Name/ | ID Code | Dilution/special |
|---|---|---|---|
| TA # | Description | (Batch/Lot#) | handling* |
| 1. | Activated Silk 27P | TOX182-76/ | Use as supplied. |
| | (in carrier serum) | SEN182-79 | Occlusive patch. |

Negative Control—0.9% Sodium Chloride (NaCl—Phototoxicity only)

### Test Material Preparation and Application

Test materials were and applied to the occlusive patch as follows:
Liquids: Approximately 0.15 mL of the test material was applied to the fabric portion of the appropriate adhesive patch.
Fabrics and Similar Materials: Test materials were into ¾-inch (approximately 2 cm)×¾-inch (approximately 2 cm) pieces and placed on the fabric portion of the appropriate patch.
Solids: Approximately 0.15 g of the test material was applied to the fabric portion of the appropriate patch.
Powders: Approximately 0.015 g of the test material was applied to the fabric portion of the appropriate patch.

### Patch Descriptions

Occlusive Strip—Patches consisting of 5 cm wide strips of occlusive Blenderm® (3M Co) tape to which Webril® (Kendall Corporation) disks, approximately 2 cm square were fixed along the midline.

### Study Evaluations and Instrumentation

### Light Source

UV radiation was obtained from a 300W multiport solar simulator Model 601 V2.5 (Solar Light Company, Glenside PA). The unit was allowed to warm up for 20 minutes before use. The following filters were used to ensure the proper spectral outputs: Schott WG320/1 mm and UG11/1 mm.

During the Induction Phase of the photoallergy portion of the study, designated sites were irradiated with two times the subject's MED (full spectrum wavelengths UVB 290-320 nm and UVA 320-400 nm). During the Challenge Phase, designated sites were irradiated with a non-erythrogenic dose of UVA radiation (wavelengths 320-400 nm) equivalent to approximately 10 J/cm². A Schott WG-345 filter was used to block UVB wavelength 290-320 nanometers.

For evaluation of phototoxicity, a Schott WG-345 filter was used to block UVB wavelength 290-320 nanometers. Sites were irradiated with long wave (UVA 320-400 nm) radiation equivalent to approximately 10 J/cm².

### Test Sites

The area to be tested was the back between the belt line and shoulder blade, lateral to the midline.

### Minimal Erythema Dose (Med)

A MED is defined as the lowest ultraviolet (UV) dose that produces the first perceptible, unambiguous erythema with defined borders appearing over most of the field of UV exposure, 16 to 24 hours after exposure. Each subject participated in a two-day procedure to determine initial minimal erythema dose (MED). Prior to the testing phase, the MED of each subject was determined by a progressive geometric sequence of UV radiation exposures to five sub-sites, each of which was graduated incrementally by 25% over that of the previous site.

| Dose 1 | Dose 2 | Dose 3 | Dose 4 | Dose 5 |
|---|---|---|---|---|
| 0.64× | 0.80× | 1.00× | 1.25× | 1.56× |

Approximately 16 to 24 hours after irradiation (Day 2), the sites were evaluated to determine the MED according to the following scoring system:
   0 Negative, no visible reaction
   ±Minimal erythema, the first perceptible redness reaction with clearly defined borders
   1+ Defined erythema
   2+ Moderate erythema
   3+ Severe erythema

### Dermal Grading Scale

The following Dermal Scoring System was used to evaluate the irradiated area within the treated site.

TABLE 64
Dermal Scoring

| Score | Description | Letter Codes |
|---|---|---|
| 0 | No visible skin reaction | e = Edema |
| + | Barely perceptible erythema | P = Peeling |
| 1+ | Mild erythema | S = Spreading of reaction |
| 2+ | Well defined erythema | beyond irradiated site. |
| 3+ | Severe erythema and edema | Sc = Scabbing |
| 4+ | Erythema and edema with vesiculation | |

d = Dryness/scaling D = Oozing, crusting, and/or superficial erosions
I = Itching Pa = Papules V = Vesicle
W = Weeping Hr = Hyperpigmentation Ho = Hypopigmentation
M = Missed Visit

For photoallergy testing, the skin condition of the test sites was evaluated at each visit. The irradiated area within the treated site was evaluated approximately 24 hours after light exposure, and a score was assigned and recorded. Any other reaction on the treated/non-irradiated site and outside the irradiated area was recorded accordingly. If a grade≥2+ erythema was observed on a site as cumulative effect of a test material, with or without UV exposure, the subject was excused by the Principal Investigator from further exposure to the test material and artificial light irradiation.
For phototoxicity testing: If a grade≥2+ erythema was observed after patch removal prior to irradiation, the subject was discontinued by the Principal Investigator from the study.

### Photoallergy

### Induction Phase

Subjects had one test material applied to the test sites. Subjects arrived at the test center on the day of study initiation. Inclusion/exclusion criteria was verified, and informed consent obtained. Visual assessment of the skin of each subject's back was performed. Qualified subjects were enrolled. A designated area on the back was irradiated on Day 1 and scored the following day to confirm each subject's historical MED.

Duplicate test sites on the back (each approximately 4 cm2) were identified. The test material was applied to designated patches and affixed to both sites on the back. One site was designated for irradiation, and the other was used as a treated, non-irradiated control.

Approximately 24 hours later, the patches were removed, the skin was gently wiped, and the test sites was evaluated according to the dermal scoring scale listed in Section 8.4. The site designated for MED determination was graded according to the dermal scoring scale listed in Section 8.3. One test site was irradiated with two times the subject's MED. The remaining site served as the non-irradiated control. Test sites were delineated with a marking pen to ensure continuity of patch application and patch site location.

This procedure was carried out twice weekly for a total of six patch applications and post-irradiation readings. However, the schedule could be modified to accommodate inclement weather, holidays, or missed patch applications and/or irradiations. At the discretion of the Principal Investigator, the test material could then be applied, the sites irradiated and scored on two consecutive days during the Induction Phase. If a patch application and/or irradiation were missed, a 3-day sequence of patch/rradiation/score could be added at the end of the Induction Phase. Subjects would have no fewer than 5 evaluations at the end of the Induction phase. Only one rescheduled patch application or irradiation procedure could be permitted. Subjects missing a second visit were discontinued from the study due to non-compliance with the study protocol.

Irradiated areas within the treated sites and the entire treated area of the control sites (treated/non-irradiated) were examined approximately 24 hours following irradiation of the test sites and graded as described in Section 8.4. Skin condition of the sites were evaluated at each visit for possible skin reactions to the test material. Any reaction was documented on the source document.

If a 2+ reaction or greater was to occur on a site, the application of the test material would be discontinued for the remainder of the Induction Phase but could be challenged on the appropriate day of the study.

At the discretion of the Principal Investigator, subjects exhibiting a significant reaction at the beginning of the Induction Phase could be considered to be “pre-sensitized” to an ingredient(s) of the test product and could be discontinued from the patching and irradiation of that test material for the remainder of the study.

### Challenge

Approximately 10 to 21 days following the last application, duplicate patches were applied to sites previously unexposed to the test material. Approximately 24 hours later, the patches were removed, and one test site was irradiated with a non-erythrogenic dose of UVA radiation (wavelengths 320-400 nm) equivalent to approximately 10 J/cm². The second test site served as a test material treated, non-irradiated control site. An additional untreated test site was also irradiated (control site). The challenge sites was graded at 24-, 48-, and 72-hours following irradiation according to the grading scale. Data of subjects with only two challenge phase scores were only considered valid if the last evaluation was performed at 72 hours post irradiation. The schedule could be modified to accommodate inclement weather.

TABLE 65
Table study schedule

| | | | | | | | | | | | | | | | | Challenge Phase | | | | |
|---|---|---|---|---|---|---|---|---|---|---|---|---|---|---|---|---|---|---|---|---|
| | Induction Phase | | | | | | | | | | | | | | | | | 24 h | 48 h | 72 h |
| | Visit | | | | | | | | | | | | | | | | | | | |
| Procedure | 1 | 2 | 3 | 4 | 5 | 6 | 7 | 8 | 9 | 10 | 11 | 12 | 13 | 14 | 15 | 16 | 17 | 18 | 19 | 20 |
| Informed Consent | X | | | | | | | | | | | | | | | | | | | |
| obtained | | | | | | | | | | | | | | | | | | | | |
| Inclusion/exclusion | X | | | | | | | | | | | | | | | | | | | |
| criteria verified | | | | | | | | | | | | | | | | | | | | |
| Confirmatory MED | X | X | | | | | | | | | | | | | | | | | | |
| Evaluation | | | | | | | | | | | | | | | | | | | | |
| Test sites examined | X | | | | | | | | | | | | | | | | | | | |
| for eligibility | | | | | | | | | | | | | | | | | | | | |
| Patches applied | X | | X | | | X | | X | | | X | | X | | | X † | | | | |
| Patches removed | | X | | X | | | X | | X | | | X | | X | | | X | | | |
| Specified sites | | X | | X | | | X | | X | | | X | | X | | | X | | | |
| irradiated | | | | | | | | | | | | | | | | | | | | |
| Sites graded for | X | X | X | X | X | X | X | X | X | X | X | X | X | X | X | X | X | X | X | X |
| dermal irritation | | | | | | | | | | | | | | | | | | | | |

Three patches would be applied to the subject's back and allowed to remain in direct contact with the skin for 24 hours. The subject would return after 24 hours for removal of the patch and grading of the site, without irradiation if sensitization is suspected, or with irradiation of an additional treated site (24-hour exposure) if reactions suggestive of photosensitization are observed during the study. Subjects would return for a 48-hour and 72-hour post-patch application grading and additional 96-hour evaluation if reactions persisted. If the sites are irradiated, subjects would return for evaluations at 24, 48 and 72 hours post irradiation.

### Phototoxicity

12 subjects participated and completed the evaluation of phototoxic potential study. These subjects had the test material applied under additional patches during the Photoallergy Induction Phase when a five-day schedule was possible.

Approximately 24 hours after application, the patch was removed, the skin was gently wiped with water and cotton balls to remove excess product, and the test site was evaluated according to the dermal scoring scale. The site was irradiated with long wave (UVA 320-400 nm) radiation (equivalent to approximately 10 J/cm2). An adjacent, untreated site (0.9% NaCl) was also irradiated (control site). Treated and control sites were examined at 24-, 48-, and 72-hours post-irradiation and graded according to the scale listed in Section 8.4. The treated non-irradiated site from the photoallergy evaluation served as a control for the phototoxicity portion of the study.

Data of subjects with only two scores post-irradiation were only considered valid if the scores at 24 and 48 hours were obtained.

TABLE 66
Procedure Schedule

| Procedure | | | 24 h | 48 h | 72 h |
|---|---|---|---|---|---|
| Visit | 1 | 2 | 3 | 4 | 5 |
| Patch applied | X | | | | |
| Patch removed | | X | | | |
| Treated and untreated (0.9% NaCl) | | X | | | |
| sites are irradiated | | | | | |
| All sites graded for dermal irritation | X | X | X | X | X |

### Photoallergy: Induction Phase and Challenge Phase Results

TABLE 67
INDIVIDUAL DERMAL SCORES FOR TEST ARTICLE 1: Activated Silk 27P (in carrier serum) (TOX182-76/SEN182-79)

| | DERMAL SCORES | | | | | | | | | | | | | | | | | | | | |
|---|---|---|---|---|---|---|---|---|---|---|---|---|---|---|---|---|---|---|---|---|---|
| | Induction Phase | | | | | | | | | | | | | | | | | | | | |
| | Evaluation 24 Hours Post-Irradiation | | | | | | | | | | | | Challenge Phase Post-Irradiation | | | | | | | | |
| Subject | 1 | | 2 | | 3 | | 4 | | 5 | | 6 | | 24 Hour | | | 48 Hour | | | 72 Hour | | |
| Number | I | NI | I | NI | I | NI | I | NI | I | NI | I | NI | I | NI | C | I | NI | C | I | NI | C |
| 1 | 0 | 0 | 0 | 0 | 0 | 0 | 0 | 0 | 0 | 0 | 0 | 0 | 0 | 0 | 0 | 0 | 0 | 0 | 0 | 0 | 0 |
| 2 | 0 | 0 | 0 | 0 | 0 | 0 | 0 | 0 | 0 | 0 | 0 | 0 | 0 | 0 | 0 | 0 | 0 | 0 | 0 | 0 | 0 |
| 3 | 0 | 0 | 0 | 0 | 0 | 0 | 0 | 0 | 0 | 0 | 0 | 0 | 0 | 0 | 0 | 0 | 0 | 0 | 0 | 0 | 0 |
| 4 | 0 | 0 | 0 | 0 | 0 | 0 | 0 | 0 | 0 | 0 | 0 | 0 | 0 | 0 | 0 | 0 | 0 | 0 | 0 | 0 | 0 |
| 5 | 0 | 0 | 0 | 0 | 0 | 0 | 0 | 0 | 0 | 0 | 0 | 0 | 0 | 0 | 0 | 0 | 0 | 0 | 0 | 0 | 0 |
| 6 | 0 | 0 | 0 | 0 | 0 | 0 | 0 | 0 | 0 | 0 | 0 | 0 | 0 | 0 | 0 | 0 | 0 | 0 | 0 | 0 | 0 |
| 7 | 0 | 0 | 0 | 0 | 0 | 0 | 0 | 0 | 0 | 0 | 0 | 0 | 0 | 0 | 0 | 0 | 0 | 0 | 0 | 0 | 0 |
| 8 | 0 | 0 | 0 | 0 | 0 | 0 | 0 | 0 | 0 | 0 | 0 | 0 | 0 | 0 | 0 | 0 | 0 | 0 | 0 | 0 | 0 |
| 9 | 0 | 0 | 0 | 0 | 0 | 0 | 0 | 0 | 0 | 0 | 0 | 0 | 0 | 0 | 0 | 0 | 0 | 0 | 0 | 0 | 0 |
| 10 | 0 | 0 | 0 | 0 | 0 | 0 | 0 | 0 | 0 | 0 | 0 | 0 | 0 | 0 | 0 | 0 | 0 | 0 | 0 | 0 | 0 |
| 11 | 0 | 0 | 0 | 0 | 0 | 0 | 0 | 0 | 0 | 0 | 0 | 0 | 0 | 0 | 0 | 0 | 0 | 0 | 0 | 0 | 0 |
| 12 | 0 | 0 | 0 | 0 | 0 | 0 | 0 | 0 | 0 | 0 | 0 | 0 | 0 | 0 | 0 | 0 | 0 | 0 | 0 | 0 | 0 |
| 13 | 0 | 0 | 0 | 0 | 0 | 0 | 0 | 0 | 0 | 0 | 0 | 0 | 0 | 0 | 0 | 0 | 0 | 0 | 0 | 0 | 0 |
| 14 | 0 | 0 | 0 | 0 | 0 | 0 | 0 | 0 | 0 | 0 | 0 | 0 | 0 | 0 | 0 | 0 | 0 | 0 | 0 | 0 | 0 |
| 15 | 0 | 0 | 0 | 0 | 0 | 0 | 0 | 0 | 0 | 0 | 0 | 0 | 0 | 0 | 0 | 0 | 0 | 0 | 0 | 0 | 0 |
| 16 | 0 | 0 | 0 | 0 | 0 | 0 | 0 | 0 | 0 | 0 | 0 | 0 | 0 | 0 | 0 | 0 | 0 | 0 | 0 | 0 | 0 |
| 17 | 0 | 0 | 0 | 0 | 0 | 0 | 0 | 0 | 0 | 0 | 0 | 0 | 0 | 0 | 0 | 0 | 0 | 0 | 0 | 0 | 0 |
| 18 | 0 | 0 | 0 | 0 | 0 | 0 | 0 | 0 | 0 | 0 | 0 | 0 | 0 | 0 | 0 | 0 | 0 | 0 | 0 | 0 | 0 |
| 19 | 0 | 0 | 0 | 0 | ± | 0 | ± | 0 | ± | 0 | 0 | 0 | 0 | 0 | 0 | 0 | 0 | 0 | 0 | 0 | 0 |
| 20 | 0 | 0 | 0 | 0 | 0 | 0 | 0 | 0 | 0 | 0 | 0 | 0 | 0 | 0 | 0 | 0 | 0 | 0 | 0 | 0 | 0 |
| 21 | 0 | 0 | 0 | 0 | 0 | 0 | 0 | 0 | 0 | 0 | 0 | 0 | 0 | 0 | 0 | 0 | 0 | 0 | 0 | 0 | 0 |
| 22 | 0 | 0 | 0 | 0 | 0 | 0 | 0 | 0 | 0 | 0 | 0 | 0 | 0 | 0 | 0 | 0 | 0 | 0 | 0 | 0 | 0 |
| 23 | 0 | 0 | 0 | 0 | ± | 0 | ± | 0 | ± | 0 | ± | 0 | 0 | 0 | 0 | 0 | 0 | 0 | 0 | 0 | 0 |
| 24 | 0 | 0 | 0 | 0 | 0 | 0 | 0 | 0 | 0 | 0 | 0 | 0 | 0 | 0 | 0 | 0 | 0 | 0 | 0 | 0 | 0 |
| 25 | 0 | 0 | 0 | 0 | D/O | D/O | D/O | D/O | D/O | D/O | D/O | D/O | D/O | D/O | D/O | D/O | D/O | D/O | D/O | D/O | D/O |
| 26 | 0 | 0 | 0 | 0 | 0 | 0 | 0 | 0 | 0 | 0 | 0 | 0 | 0 | 0 | 0 | 0 | 0 | 0 | 0 | 0 | 0 |
| 27 | 0 | 0 | 0 | 0 | 0 | 0 | 0 | 0 | 0 | 0 | 0 | 0 | 0 | 0 | 0 | 0 | 0 | 0 | 0 | 0 | 0 |
| 28 | 0 | 0 | 0 | 0 | 0 | 0 | 0 | 0 | 0 | 0 | 0 | 0 | 0 | 0 | 0 | 0 | 0 | 0 | 0 | 0 | 0 |
| 29 | 0 | 0 | 0 | 0 | 0 | 0 | 0 | 0 | 0 | 0 | 0 | 0 | 0 | 0 | 0 | 0 | 0 | 0 | 0 | 0 | 0 |
| 30 | 0 | 0 | 0 | 0 | 0 | 0 | 0 | 0 | 0 | 0 | 0 | 0 | 0 | 0 | 0 | 0 | 0 | 0 | 0 | 0 | 0 |

TABLE 68
INDIVIDUAL DERMAL SCORES FOR TEST ARTICLE 1: Activated
Silk 27P (in carrier serum) (TOX182-76/SEN182-79)

| Subject | 24 Hour | | | 48 Hour | | | 72 Hour | | |
|---|---|---|---|---|---|---|---|---|---|
| Number | I | NI | C | I | NI | C | I | NI | C |
| 1 | 0 | 0 | 0 | 0 | 0 | 0 | 0 | 0 | 0 |
| 2 | 0 | 0 | 0 | 0 | 0 | 0 | 0 | 0 | 0 |
| 3 | 0 | 0 | 0 | 0 | 0 | 0 | 0 | 0 | 0 |
| 4 | 0 | 0 | 0 | 0 | 0 | 0 | 0 | 0 | 0 |
| 5 | 0 | 0 | 0 | 0 | 0 | 0 | 0 | 0 | 0 |
| 6 | 0 | 0 | 0 | 0 | 0 | 0 | 0 | 0 | 0 |
| 7 | 0 | 0 | 0 | 0 | 0 | 0 | 0 | 0 | 0 |
| 8 | 0 | 0 | 0 | 0 | 0 | 0 | 0 | 0 | 0 |
| 9 | 0 | 0 | 0 | 0 | 0 | 0 | 0 | 0 | 0 |
| 10 | 0 | 0 | 0 | 0 | 0 | 0 | 0 | 0 | 0 |
| 11 | 0 | 0 | 0 | 0 | 0 | 0 | 0 | 0 | 0 |
| 12 | 0 | 0 | 0 | 0 | 0 | 0 | 0 | 0 | 0 |

### 19. Conclusions

Individual scores for photoallergy are presented in Table 67. No dermal irritation was observed during the Challenge Phase.

Individual scores for phototoxicity are presented in Table 68. No dermal irritation was observed at the 24-hour, 48-hour, and 72-hour visits.

Under the conditions of this study and in this test population, the test material identified as Activated Silk 27P (in carrier serum) (TOX182-76/SEN182-79) did not produce any phototoxic or photoallergic responses.

### Example 28: Collagen Redux HRIPT Test

Purpose: The purpose of this example was to investigate the irritation and sensitization potential of the Collagen Redux Serum (formerly named 27P serum).

Scope: Evolved by Nature is launching the Collagen Redux Serum in 2023. A Repeat Insult Patch Test was completed on 106 healthy volunteers to validate product safety and for claims substantiation.

### Test Materials:

Collagen Redux Serum/27P Serum Reference #: 172-5-1

### Study Protocol:

106 subjects completed the study with their age & gender being recorded. 50% of subjects had self-assessed sensitive skin. A total of nine induction patches were worn for 47 or 71 hours for three weeks. Subjects had a 14 day rest period. Challenge patches were applied for 48 hours and readings were made at 1 hour and 48 hours post removal.

### Results

Collagen Redux Serum/27P serum showed no visible erythematous reactions during the Induction phase of the study. There were no questionable reactions observed during the Challenge Phase by any of the subjects to any of the test materials.

### Conclusions

The test article can be considered as safe for use under the conditions of the study, and claims such as, “Clinically Tested”, “Kind to Skin”, “Safe for Skin”, “Safe for Sensitive Skin,” “Dermatologist Approved” and “ ” Hypoallergenic” are substantiated.

### Example 29: Comparative Silk Protein (e.g., Silk Fibroin) Processing Parameters

TABLE 69
Processing Parameters

| | | | | | | | Example of Specific Conditions | | | | | |
|---|---|---|---|---|---|---|---|---|---|---|---|---|
| | | | | | | | and Ranges to Create Specific | | | | | |
| | | | | | | | Fibroin Fragment Populations | | | | | |
| | | | | | | | that have a single weight average | | | | | |
| | | | | | | | Molecular Weight Average (MW) | | | | | |
| | | | | | | | and poly dispersity below 3 | | | | | |
| Unit Op | Process Parameter | Parameter | Gradient | Standard | | Available | “Mid | | “Low | | “High | |
| | | Variables and | (if | Dev | | in | Range” of | | Range” of | | Range” of | |
| | | Process | applicable) | (if | | original | specific | | specific | | specific | |
| | | Operating | | applicable) | | BT | single MW | | single MW | | single MW | |
| | | Ranges that | | | | process | fragment | | fragment | | fragment | |
| | | can be used to | | | | | populations | | populations | | populations | |
| | | create single | | | | | created | | created | | created | |
| | | specific weight | | | | | from range | | from range | | from range | |
| | | average | | | | | of process | | of process | | of process | |
| | | Molecular | | | | | conditions: | | conditions: | | conditions: | |
| | | Weight | | | | | 39.7 kDa | | 27.2 kDa | | 95.1 kDa | |
| | | Average (MW) | | | | | single | | single | | single | |
| | | Fragment | | | | | poplulation | | poplulation | | poplulation | |
| | | Populations | | | | | to 33.0 kDA | | to 11.9 kDA | | to 67.6 kDA | |
| | | Modifications | | | | | single MW | | single MW | | single MW | |
| | | as determined | | | | | population | | population | | population | |
| | | by either | | | | | by SEC-RI | | by SEC-RI | | by SEC-MALS | |
| | | HPLC with | | | | | | | | | | |
| | | SEC-RI or | | | | | | | | | | |
| | | SEC-MALS | | | | | | | | | | |
| | | between 1 | | | | | | | | | | |
| | | kDa and 250 | | | | | | | | | | |
| | | kDa, each | | | | | | | | | | |
| | | population with | | | | | | | | | | |
| | | polydispersity | | | | | | | | | | |
| | | equal to or | | | | | | | | | | |
| | | lower than 3 | | | | | | | | | | |
| Fibroin | Equipment style | Stationary bed | — | — | | Yes | Dispersed | | Dispersed | | Dispersed | |
| isolation | | w/continuous | | | | | solid with | | solid with | | solid with | |
| or | | solvent spray, | | | | | agitated | | agitated | | agitated | |
| extraction | | moving bed | | | | | solvent. | | solvent. | | solvent. | |
| | | w/counter | | | | | Solid held | | Solid held | | Solid held | |
| | | current | | | | | within | | within | | within | |
| | | solvent feed | | | | | permeable | | permeable | | permeable | |
| | | (Bollman or | | | | | secondary | | secondary | | secondary | |
| | | Hildebrandt), | | | | | containment. | | containment. | | containment. | |
| | | stationary bed | | | | | | | | | | |
| | | w/unagitated | | | | | | | | | | |
| | | solvent, | | | | | | | | | | |
| | | dispersed | | | | | | | | | | |
| | | solid with | | | | | | | | | | |
| | | agitated | | | | | | | | | | |
| | | solvent | | | | | | | | | | |
| Fibroin | Process pressure | 0.145 psia- | — | 0.05 psia- | | Range is | atmospheric | | atmospheric | | atmospheric | |
| isolation | | 1450 psia | | 10 psia | | limited | | | | | | |
| or | | | | | | for BT | | | | | | |
| extraction | | | | | | | | | | | | |
| Fibroin | Agitation | Stationary bed | — | — | | Range is | Dispersed | | Dispersed | | Dispersed | |
| isolation | | (no | | | | limited | by drum | | by drum | | by drum | |
| or | | intentional | | | | for BT | rotation | | rotation | | rotation | |
| extraction | | agitation), | | | | | | | | | | |
| | | moving bed, | | | | | | | | | | |
| | | or dispersed | | | | | | | | | | |
| | | (fully or | | | | | | | | | | |
| | | partially) in | | | | | | | | | | |
| | | solvent | | | | | | | | | | |
| Fibroin | Agitation Duration | Intermittent; | — | — | | No | Intermittent | | Intermittent | | Intermittent | |
| isolation | or Frequency | continuous | | | | | | | | | | |
| or | | | | | | | | | | | | |
| extraction | | | | | | | | | | | | |
| Fibroin | Secondary | Yes or no | — | — | | Yes | yes | | yes | | yes | |
| isolation | containment | | | | | | | | | | | |
| or | | | | | | | | | | | | |
| extraction | | | | | | | | | | | | |
| Fibroin | Solvent | 0.1% wt- | — | 0.05% | wt | Yes | 0.94% | wt | 0.94% | wt | 0.705% | wt |
| isolation | concentration | 1% wt | | | | | | | | | | |
| or | | | | | | | | | | | | |
| extraction | | | | | | | | | | | | |
| Fibroin | Cocoon to solvent | 0.01 kg/kg to | — | 0.005 kg/kg- | | Yes | 0.042 | kg/kg | 0.042 | kg/kg | 0.068 | kg/kg |
| isolation | ratio | 0.1 kg/kg | | 0.05 kg/kg | | | | | | | | |
| or | | | | | | | | | | | | |
| extraction | | | | | | | | | | | | |
| Fibroin | Extraction solvent | 30 C.- | 0.1 C.- | 0.1 C.- | | Range is | 100 | C. | 100 | C. | 100 | C. |
| isolation | temperature | 370 C. | 10 C. | 10 C. | | limited | | | | | | |
| or | | | | | | for BT | | | | | | |
| extraction | | | | | | | | | | | | |
| Fibroin | Temperature control | Internal | — | — | | Range is | internal | | internal | | internal | |
| isolation | style | (electrical/ | | | | limited | | | | | | |
| or | | flow medium | | | | for BT | | | | | | |
| extraction | | heating/cooling | | | | | | | | | | |
| | | element), | | | | | | | | | | |
| | | external | | | | | | | | | | |
| | | (circulation of | | | | | | | | | | |
| | | heat transfer | | | | | | | | | | |
| | | fluid through | | | | | | | | | | |
| | | jacket, | | | | | | | | | | |
| | | conduction | | | | | | | | | | |
| | | against | | | | | | | | | | |
| | | electrically | | | | | | | | | | |
| | | heated jacket, | | | | | | | | | | |
| | | conduction | | | | | | | | | | |
| | | against | | | | | | | | | | |
| | | electrically | | | | | | | | | | |
| | | heated plates | | | | | | | | | | |
| | | or walls), | | | | | | | | | | |
| | | indirect | | | | | | | | | | |
| | | (vessel | | | | | | | | | | |
| | | temperature | | | | | | | | | | |
| | | not maintained, | | | | | | | | | | |
| | | but solvent is | | | | | | | | | | |
| | | heated in a | | | | | | | | | | |
| | | recirculation | | | | | | | | | | |
| | | or feed). | | | | | | | | | | |
| Fibroin | Extraction Time | 5 min- | — | 0.05 | min | Yes | 30 | min | 30 | min | 30 | min |
| isolation | | 60 min | | | | | | | | | | |
| or | | | | | | | | | | | | |
| extraction | | | | | | | | | | | | |
| Fibroin | Cocoon to rinse | 0.005 kg/kg- | — | 0.001 kg/kg- | | Yes | 0.037 | kg/kg | 0.037 | kg/kg | 0.037 | kg/kg |
| isolation | water ratio | 0.04 kg/kg | | 0.005 kg/kg | | | | | | | | |
| or | | | | | | | | | | | | |
| extraction | | | | | | | | | | | | |
| Fibroin | Rinse water | 1 C.- | 0.1 C.- | 0.1 C.- | | Range is | 60 | C. | 60 | C. | 60 | C. |
| isolation | temperature | 370 C. | 10 C. | 10 C. | | limited | | | | | | |
| or | | | | | | for BT | | | | | | |
| extraction | | | | | | | | | | | | |
| Fibroin | Rinse single stage | 5 min- | — | 0.05 | min | Yes | 10 min × 3 | | 10 min × 3 | | 10 min × 3 | |
| isolation | time | 60 min | | | | | | | | | | |
| or | | | | | | | | | | | | |
| extraction | | | | | | | | | | | | |
| Fibroin | Excess water | Centrifugal, | 0.1 C.- | 0.1 C.- | | Yes | centrifugal, | | centrifugal, | | centrifugal, | |
| isolation | removal | vacuum, | 10 C. | 10 C. | | | thermal | | thermal | | thermal | |
| or | | heating (up to | | | | | at 60 C. | | at 60 C. | | at 60 C. | |
| extraction | | 60 C.), dry air | | | | | | | | | | |
| | | flow, rinsing | | | | | | | | | | |
| | | w/volatile | | | | | | | | | | |
| | | solvent and | | | | | | | | | | |
| | | ambient | | | | | | | | | | |
| | | drying, or any | | | | | | | | | | |
| | | combination | | | | | | | | | | |
| | | of these | | | | | | | | | | |
| Fibroin | Equipment style | Dispersed | — | — | | Yes | Dispersed | | Dispersed | | Dispersed | |
| solvation | | solid | | | | | solid | | solid | | solid | |
| and | | w/agitated | | | | | w/agitated | | w/agitated | | w/agitated | |
| disolution | | solvent, | | | | | solvent | | solvent | | solvent | |
| | | stationary bed | | | | | | | | | | |
| | | w/recirculated | | | | | | | | | | |
| | | solvent | | | | | | | | | | |
| | | (compress | | | | | | | | | | |
| | | fibroin into | | | | | | | | | | |
| | | cage, pump | | | | | | | | | | |
| | | solvent such | | | | | | | | | | |
| | | that it strips | | | | | | | | | | |
| | | fibroin as it | | | | | | | | | | |
| | | passes through | | | | | | | | | | |
| | | the cage) | | | | | | | | | | |
| Fibroin | Process pressure | 0.145 psia- | — | 0.05 psia- | | Range is | atmospheric | | atmospheric | | atmospheric | |
| solvation | | 1450 psia | | 10 psia | | limited | | | | | | |
| and | | | | | | for BT | | | | | | |
| disolution | | | | | | | | | | | | |
| Fibroin | Agitation type or | Stationary bed | — | — | | Range is | Solid | | Solid | | Partially | |
| solvation | method | (no | | | | limited | fully | | fully | | dispersed | |
| and | | intentional | | | | for BT | dispersed | | dispersed | | by manual | |
| disolution | | agitation), or | | | | | in solvent | | in solvent | | agitation | |
| | | dispersed | | | | | | | | | | |
| | | (fully or | | | | | | | | | | |
| | | partially) in | | | | | | | | | | |
| | | solvent by | | | | | | | | | | |
| | | some means | | | | | | | | | | |
| Fibroin | Agitation Duration | Intermitent; | — | — | | Range is | Continuous | | Continuous | | Intermittent | |
| solvation | or Frequency | continous | | | | limited | | | | | | |
| and | | | | | | for BT | | | | | | |
| disolution | | | | | | | | | | | | |
| Fibroin | Secondary | Yes or no | — | — | | Yes | No | | No | | No | |
| solvation | containment | | | | | | | | | | | |
| and | | | | | | | | | | | | |
| disolution | | | | | | | | | | | | |
| Fibroin | Solvent | 10% wt- | — | 0.1% | wt | Yes | 51.5% wt | | 51.5% wt | | 51.5% wt | |
| solvation | concentration | 70% wt | | | | | LiBr | | LiBr | | LiBr | |
| and | | | | | | | | | | | | |
| disolution | | | | | | | | | | | | |
| Fibroin | Fibroin to solvent | 0.02 kg/kg to | — | 0.005 kg/k- | | Yes | 0.159 | kg/kg | 0.159 | kg/kg | 0.159 | kg/kg |
| solvation | ratio | 0.2 kg/kg | | 0.05 kg/kg | | | | | | | | |
| and | | | | | | | | | | | | |
| disolution | | | | | | | | | | | | |
| Fibroin | Solvent temperature | 20 C.- | 0.1 C.- | 0.1 C.- | | Range is | 100 | C. | 123 | C. | 100 | C. |
| solvation | | 370 C. | 10 C. | 10 C. | | limited | | | | | | |
| and | | | | | | for BT | | | | | | |
| disolution | | | | | | | | | | | | |
| Fibroin | Temperature control | Internal | — | — | | Range is | external | | external | | external | |
| solvation | style | (electrical/ | | | | limited | | | | | (held in | |
| and | | flow medium | | | | for BT | | | | | oven) | |
| disolution | | heating/cooling | | | | | | | | | | |
| | | element), | | | | | | | | | | |
| | | external | | | | | | | | | | |
| | | (circulation of | | | | | | | | | | |
| | | heat transfer | | | | | | | | | | |
| | | fluid through | | | | | | | | | | |
| | | jacket, | | | | | | | | | | |
| | | conduction | | | | | | | | | | |
| | | against | | | | | | | | | | |
| | | electrically | | | | | | | | | | |
| | | heated jacket, | | | | | | | | | | |
| | | conduction | | | | | | | | | | |
| | | against | | | | | | | | | | |
| | | electrically | | | | | | | | | | |
| | | heated plates | | | | | | | | | | |
| | | or walls), | | | | | | | | | | |
| | | indirect | | | | | | | | | | |
| | | (vessel | | | | | | | | | | |
| | | temperature | | | | | | | | | | |
| | | not | | | | | | | | | | |
| | | maintained, | | | | | | | | | | |
| | | but solvent is | | | | | | | | | | |
| | | heated in a | | | | | | | | | | |
| | | recirculation | | | | | | | | | | |
| | | or feed). | | | | | | | | | | |
| Fibroin | Fibroin loading time | 0 min- | — | 0 min- | | Range is | 45 | min | 45 | min | 0 | min |
| solvation | | 60 min | | 15 min | | limited | | | | | | |
| and | | | | | | for BT | | | | | | |
| disolution | | | | | | | | | | | | |
| Fibroin | Reaction time | 1 min- | — | 0.05 | min | Yes | From 1 | | From 17 | | From 15 | |
| solvation | | 1440 min | | | | | min to 63 | | min to | | min to 60 | |
| and | | | | | | | min | | 360 min | | min | |
| disolution | | | | | | | | | | | | |
| Fibroin | Cooling style | Internal (flow | — | — | | Range is | Ambient | | Ambient | | Ambient | |
| solvation | | medium | | | | limited | cooling | | cooling | | cooling | |
| and | | cooling | | | | for BT | after | | after | | after | |
| disolution | | element), | | | | | discharge | | discharge | | discharge | |
| | | external | | | | | | | | | | |
| | | (circulation of | | | | | | | | | | |
| | | heat transfer | | | | | | | | | | |
| | | fluid through | | | | | | | | | | |
| | | jacket), | | | | | | | | | | |
| | | system flow | | | | | | | | | | |
| | | through heat | | | | | | | | | | |
| | | exchanger, | | | | | | | | | | |
| | | ambient | | | | | | | | | | |
| | | cooling after | | | | | | | | | | |
| | | discharge | | | | | | | | | | |
| Fibroin | Product cooling time | 15 min- | — | 3 min- | | Range is | 1440 | min | 1440 | min | 1440 | min |
| solvation | | 1440 min | | 15 min | | limited | | | | | | |
| and | | | | | | for BT | | | | | | |
| disolution | | | | | | | | | | | | |
| Fibroin | Concentrate | Filter press, | — | — | | Range is | In-line | | In-line | | In-line | |
| purification | filtration style | shell and leaf | | | | limited | filter | | filter | | filter | |
| | (dead end) | filter, in-line | | | | for BT | | | | | | |
| | | filter, belt | | | | | | | | | | |
| | | filter, | | | | | | | | | | |
| | | centrifugal | | | | | | | | | | |
| | | (rotating | | | | | | | | | | |
| | | drum) filter, | | | | | | | | | | |
| | | vacuum filter | | | | | | | | | | |
| Fibroin | Diluted fibroin | 1% wt- | — | 0.05% | wt | Yes | 2.5% | wt | 4.5% | wt | 2.5% | wt |

| purification | concentration | 10% wt | | | | | | | |
|---|---|---|---|---|---|---|---|---|---|
| Fibroin | Diluted fibroin | Filter press, | — | — | | Range is | In-line | In-line | In-line |
| purification | filtration style | shell and leaf | | | | limited | filter | filter | filter |
| | (dead end) | filter, in-line | | | | for BT | | | |
| | | filter, belt | | | | | | | |
| | | filter, | | | | | | | |
| | | centrifugal | | | | | | | |
| | | (rotating | | | | | | | |
| | | drum) filter, | | | | | | | |
| | | vacuum filter | | | | | | | |
| Fibroin | LiBr removal style | Diafiltration | — | — | | Range is | Diafiltration | Diafiltration | Diafiltration |
| purification | | (TFF), dialysis, | | | | limited | (TFF) | (TFF) | (TFF) |
| | | adsorption | | | | for BT | | | |
| | | (chromatography) | | | | | | | |
| Fibroin | Diafiltration/ | 4 C.- | 2 C. | 2 | C. | Range is | 20 C.- | 20 C.- | 20 C.- |

| purification | concentration (TFF) | 60 C. | | | limited | 25 C. | | 25 C. | | 25 C. | |
|---|---|---|---|---|---|---|---|---|---|---|---|
| | processing | | | | for BT | | | | | | |
| | temperature | | | | | | | | | | |
| Fibroin | Temperature control | Internal | — | — | Range is | External | | External | | None | |
| purification | style | (electrical/ | | | limited | | | | | | |
| | | flow medium | | | for BT | | | | | | |
| | | heating/cooling | | | | | | | | | |
| | | element), | | | | | | | | | |
| | | external | | | | | | | | | |
| | | (circulation of | | | | | | | | | |
| | | heat transfer | | | | | | | | | |
| | | fluid through | | | | | | | | | |
| | | jacket, | | | | | | | | | |
| | | conduction | | | | | | | | | |
| | | against | | | | | | | | | |
| | | electrically | | | | | | | | | |
| | | heated jacket, | | | | | | | | | |
| | | conduction | | | | | | | | | |
| | | against | | | | | | | | | |
| | | electrically | | | | | | | | | |
| | | heated plates | | | | | | | | | |
| | | or walls), | | | | | | | | | |
| | | system flow | | | | | | | | | |
| | | through heat | | | | | | | | | |
| | | exchanger | | | | | | | | | |
| Fibroin | Diafiltration/ | 3 kDa- | — | — | Yes | 10 | kDa | 10 | kDa | 10 | kDa |
| purification | concentration (TFF) | 80 kDa | | | | | | | | | |
| | selective layer | | | | | | | | | | |
| | rejection | | | | | | | | | | |
| Fibroin | Diafiltration/ | 2 psi- | — | 0.5 psi- | Yes | 12.5 | psi | 12.5 | psi | 35 | psi |

| purification | concentration (TFF) | 40 psi | | 5 psi | | | | | | | |
|---|---|---|---|---|---|---|---|---|---|---|---|
| | TMP | | | | | | | | | | |
| Fibroin | RO membrane | 20-100 psi | — | 5-10 | psi | no | — | | — | | — |
| purification | | | | | | | | | | | |
| Fibroin | Diafiltration/ | 200 lpm- | — | 5 | lpm | No | 300 | lpm | 300 | lpm | Variable |

| purification | concentration (TFF) | 900 lpm | | | | | | | | (control | |
|---|---|---|---|---|---|---|---|---|---|---|---|
| | flow rate | | | | | | | | | to TMP) | |
| Fibroin | Concentration style | TFF, | | | Range is | TFF | | TFF | | TFF | |
| purification | | pervaporation, | — | — | limited | | | | | | |
| | | thin film | | | for BT | | | | | | |
| | | evaporation | | | | | | | | | |
| | | (vacuum, | | | | | | | | | |
| | | thermal, or | | | | | | | | | |
| | | both), reverse | | | | | | | | | |
| | | osmosis, dead | | | | | | | | | |
| | | end membrane | | | | | | | | | |
| | | filtration | | | | | | | | | |
| Fibroin | Product | 5% wt- | — | 1% | Range is | 6% | wt | 6% | wt | 6% | wt |

| | | | | | | |
|---|---|---|---|---|---|---|
| purification | concentration | 40% wt | limited | | | |
| | | | for BT | | | |
| Fibroin | Secondary | size exclusion | | NA | NA | NA |
| purification | separation | column; | | | | |
| | | RODI | | | | |
| | | membrane | | | | |
| | | type | | | | |

| | Example of Specific Conditions | | | | | | | | | |
|---|---|---|---|---|---|---|---|---|---|---|
| | to Create Specific | | | | | | | | | |
| | Fibroin Fragment Populations | | | | | | | | | |
| | with a single weight average | | | | | | | | | |
| | Molecular Weight Average (MW) | | | | | | | | | |
| | and poly dispersity below 3 | | | | | | | | | |
| Unit Op | Specific | | Specific | | Specific | | Specific | | Specific | |
| | single | | single | | single | | single | | single | |
| | “Mid” MW | | “Low” MW | | “High” MW | | “High” MW | | ultra | |
| | fragment | | fragment | | fragment | | fragment | | “Low” MW | |
| | population: | | population: | | population: | | population: | | fragment | |
| | 47 kDa by | | 22 kDa by | | 53 kDa by | | 145 kDa by | | population: | |
| | SEC-RI | | SEC-RI | | SEC-RI | | SEC-RI | | 1 kDa by | |
| | and 84 kDa | | and 37 kDa | | and 95 kDa | | and 250 kDa | | SEC-RI | |
| | by SEC-MALS | | by SEC-MALS | | by SEC-MALS | | by SEC-MALS | | and 1 kDa | |
| Fibroin | Dispersed | | Dispersed | | Dispersed | | Dispersed | | Dispersed | |
| isolation | solid with | | solid with | | solid with | | solid with | | solid with | |
| or | agitated | | agitated | | agitated | | agitated | | agitated | |
| extraction | solvent. | | solvent. | | solvent. | | solvent. | | solvent. | |
| | Solid held | | Solid held | | Solid held | | Solid held | | Solid held | |
| | within | | within | | within | | within | | within | |
| | permeable | | permeable | | permeable | | permeable | | permeable | |
| | secondary | | secondary | | secondary | | secondary | | secondary | |
| | containment. | | containment. | | containment. | | containment. | | containment. | |
| Fibroin | atmospheric | | atmospheric | | atmospheric | | atmospheric | | atmospheric | |
| isolation | | | | | | | | | | |
| or | | | | | | | | | | |
| extraction | | | | | | | | | | |
| Fibroin | Dispersed | | Dispersed | | Dispersed | | Dispersed | | Dispersed | |
| isolation | by drum | | by drum | | by drum | | by drum | | by drum | |
| or | rotation | | rotation | | rotation | | rotation | | rotation | |
| extraction | | | | | | | | | | |
| Fibroin | Intermittent | | Intermittent | | Intermittent | | Intermittent | | Intermittent | |
| isolation | | | | | | | | | | |
| or | | | | | | | | | | |
| extraction | | | | | | | | | | |
| Fibroin | yes | | yes | | yes | | yes | | yes | |
| isolation | | | | | | | | | | |
| or | | | | | | | | | | |
| extraction | | | | | | | | | | |
| Fibroin | 0.94% | wt | 0.94% | wt | 0.705% | wt | 0.705% | wt | 1.88% | wt |
| isolation | | | | | | | | | | |
| or | | | | | | | | | | |
| extraction | | | | | | | | | | |
| Fibroin | 0.042 | kg/kg | 0.042 | kg/kg | 0.068 | kg/kg | 0.068 | kg/kg | 0.042 | kg/kg |
| isolation | | | | | | | | | | |
| or | | | | | | | | | | |
| extraction | | | | | | | | | | |
| Fibroin | 100 | C. | 100 | C. | 100 | C. | 60 | C. | 100 | C. |
| isolation | | | | | | | | | | |
| or | | | | | | | | | | |
| extraction | | | | | | | | | | |
| Fibroin | internal | | internal | | internal | | internal | | internal | |
| isolation | | | | | | | | | | |
| or | | | | | | | | | | |
| extraction | | | | | | | | | | |
| Fibroin | 30 | min | 30 | min | 30 | min | 15 | min | 60 | min |
| isolation | | | | | | | | | | |
| or | | | | | | | | | | |
| extraction | | | | | | | | | | |
| Fibroin | 0.037 | kg/kg | 0.037 | kg/kg | 0.037 | kg/kg | 0.037 | kg/kg | 0.037 | kg/kg |
| isolation | | | | | | | | | | |
| or | | | | | | | | | | |
| extraction | | | | | | | | | | |
| Fibroin | 60 | C. | 60 | C. | 60 | C. | 60 | C. | 60 | C. |
| isolation | | | | | | | | | | |
| or | | | | | | | | | | |
| extraction | | | | | | | | | | |
| Fibroin | 10 min × 3 | | 10 min × 3 | | 10 min × 3 | | 10 min × 3 | | 10 min × 3 | |
| isolation | | | | | | | | | | |
| or | | | | | | | | | | |
| extraction | | | | | | | | | | |
| Fibroin | centrifugal, | | centrifugal, | | centrifugal, | | centrifugal, | | centrifugal, | |
| isolation | thermal | | thermal | | thermal | | thermal | | thermal | |
| or | at 60 C. | | at 60 C. | | at 60 C. | | at 60 C. | | at 60 C. | |
| extraction | | | | | | | | | | |
| Fibroin | Dispersed | | Dispersed | | Dispersed | | Dispersed | | Dispersed | |
| solvation | solid | | solid | | solid | | solid | | solid | |
| and | w/agitated | | w/agitated | | w/agitated | | w/agitated | | w/agitated | |
| disolution | solvent | | solvent | | solvent | | solvent | | solvent | |
| Fibroin | atmospheric | | atmospheric | | atmospheric | | atmospheric | | atmospheric | |
| solvation | | | | | | | | | | |
| and | | | | | | | | | | |
| disolution | | | | | | | | | | |
| Fibroin | Solid | | Solid | | Partially | | Partially | | Solid | |
| solvation | fully | | fully | | dispersed | | dispersed | | fully | |
| and | dispersed | | dispersed | | by manual | | by manual | | dispersed | |
| disolution | in solvent | | in solvent | | agitation | | agitation | | in solvent | |
| Fibroin | Continuous | | Continuous | | Intermittent | | Intermittent | | Continuous | |
| solvation | | | | | | | | | | |
| and | | | | | | | | | | |
| disolution | | | | | | | | | | |
| Fibroin | No | | No | | No | | No | | No | |
| solvation | | | | | | | | | | |
| and | | | | | | | | | | |
| disolution | | | | | | | | | | |
| Fibroin | 51.5% wt | | 51.5% wt | | 51.5% wt | | 51.5% wt | | 51.5% wt | |
| solvation | LiBr | | LiBr | | LiBr | | LiBr | | LiBr | |
| and | | | | | | | | | | |
| disolution | | | | | | | | | | |
| Fibroin | 0.159 | kg/kg | 0.159 | kg/kg | 0.159 | kg/kg | 0.159 | kg/kg | 0.159 | kg/kg |
| solvation | | | | | | | | | | |
| and | | | | | | | | | | |
| disolution | | | | | | | | | | |
| Fibroin | 100 | C. | 123 | C. | 100 | C. | 60 | C. | 135 C. | |
| solvation | | | | | | | | | (boiling) | |
| and | | | | | | | | | | |
| disolution | | | | | | | | | | |
| Fibroin | external | | external | | external | | external | | external | |
| solvation | | | | | (held in | | (held in | | | |
| and | | | | | oven) | | oven) | | | |
| disolution | | | | | | | | | | |
| Fibroin | 45 | min | 45 | min | 0 | min | 0 | min | 45 | min |
| solvation | | | | | | | | | | |
| and | | | | | | | | | | |
| disolution | | | | | | | | | | |
| Fibroin | 20 | min | 46 | min | 15 | min | 15 | min | 480 | min |
| solvation | | | | | | | | | | |
| and | | | | | | | | | | |
| disolution | | | | | | | | | | |
| Fibroin | Ambient | | Ambient | | Ambient | | Ambient | | Ambient | |
| solvation | cooling | | cooling | | cooling | | cooling | | cooling | |
| and | after | | after | | after | | after | | after | |
| disolution | discharge | | discharge | | discharge | | discharge | | discharge | |
| Fibroin | 1440 | min | 1440 | min | 1440 | min | 1440 | min | 1440 | min |
| solvation | | | | | | | | | | |
| and | | | | | | | | | | |
| disolution | | | | | | | | | | |
| Fibroin | In-line | | In-line | | In-line | | In-line | | In-line | |
| purification | filter | | filter | | filter | | filter | | filter | |
| Fibroin | 2.5% | wt | 4.5% | wt | 2.5% | wt | 2.5% | wt | 4.5% | wt |
| purification | | | | | | | | | | |
| Fibroin | In-line | | In-line | | In-line | | In-line | | In-line | |
| purification | filter | | filter | | filter | | filter | | filter | |
| Fibroin | Diafiltration | | Diafiltration | | Diafiltration | | Diafiltration | | Diafiltration | |
| purification | (TFF) | | (TFF) | | (TFF) | | (TFF) | | (TFF) | |
| Fibroin | 20 C.- | | 20 C.- | | 20 C.- | | 20 C.- | | 30 C.- | |
| purification | 25 C. | | 25 C. | | 25 C. | | 25 C. | | 40 C. | |
| Fibroin | External | | External | | None | | None | | External | |
| purification | | | | | | | | | | |
| Fibroin | 10 | kDa | 10 | kDa | 10 | kDa | 10 | kDa | RO | |
| purification | | | | | | | | | membrane | |
| | | | | | | | | | (salt | |
| | | | | | | | | | rejection | |
| | | | | | | | | | only) | |
| Fibroin | 12.5 | psi | 12.5 | psi | 35 | psi | 35 | psi | — | |
| purification | | | | | | | | | | |
| Fibroin | — | | — | | — | | — | | 70 | psi |
| purification | | | | | | | | | | |
| Fibroin | 300 | lpm | 300 | lpm | Variable | | Variable | | 300 | lpm |
| purification | | | | | (control | | (control | | | |
| | | | | | to TMP) | | to TMP) | | | |
| Fibroin | TFF | | TFF | | TFF | | TFF | | TFF | |
| purification | | | | | | | | | | |
| Fibroin | 6% | wt | 6% | wt | 6% | wt | 6% | wt | 6% | wt |

| | | | | | |
|---|---|---|---|---|---|
| purification | | | | | |
| Fibroin | NA | NA | NA | NA | NA |
| purification | | | | | |

### Example 30: Construction of Silk Fibroin-Nanoclay Composite Films and/or Coatings, Used to Regulate Water Vapor (and Other Gas) Permeability Across the Material Surface

This example encompasses the construction of silk fibroin-nanoclay composite films and/or coatings, used to regulate water vapor (and other gas) permeability across the material surface.

Films composed of regenerated silk fibroin (RSF) and nanoclays such as bentonite clay exhibit lower water vapor permeability (WVP) properties compared to films cast using equivalent quantities of RSF or nanoclay alone. The mechanism through which incorporation of bentonite clay reduces WVP will also serve to reduce the permeability of other gases. The extremely low permeability of the nanoclay platelets, which act as a filler in the RSF film matrix, increases the diffusive pathway for any permeant through the RSF/nanoclay membrane. While properties of polymer-nanoclay composites are well-documented, this disclosure represents a novel characterization of regulation of water vapor permeability specific to an RSF/nanoclay composite.

The type of clay used for this discovery include both Elementis Bentone Hydroclay 1100 (1100) and Elementis Bentone Hydroclay 2001 (2001), both of which are forms of bentonite clay. Throughout this disclosure, the term “nanoclay” will serve to encompass the general class of bentonite clays.

Synthetic polymers are currently used as liquid bandage materials in products such as Nexcare Liquid Bandage, New Skin Liquid Bandage, Curad Flex Seal, and others. Naturally occurring polymers (such as silk fibroin) are desirable for their increased biocompatibility, biodegradability, and good mechanical strength. One of the main ways that a liquid bandage promotes wound healing is to regulate the rate of WVP from damaged skin or an open wound. In a liquid bandage application, this invention could be used to target a specific WVP for an RSF/nanoclay liquid bandage film at a given thickness.

Such a reduction in permeability can be applied anywhere improved barrier properties are desirable, not just as a liquid bandage. Other potential applications include corrosion resistant coatings, flame retardant coatings, topical skin ointments, biodegradable food packaging, and others.

Additional additives such as plasticizing agents can be incorporated into silk-nanoclay materials across a range of pH conditions to augment material properties (such as strength, flexibility, etc), further expanding their potential applications.

### Properties

Films can be prepared with water-based solutions of RSF and clay. A solution with the desired concentration of both silk and clay is made by combining stock solutions of silk fibroin and clay. The clay is dissolved into water via a high shear mixer. A centrifuge may be used for controlled acidic solutions (pH<4.0), but it is not necessary when the solution is neutral (pH˜7.0). Once the desired solution is obtained, the solution is cast onto a PDMS slab and dried in an environmental chamber at 35 C, 50%/RH.

Water vapor permeability was benchmarked for silk-only and clay-only films using a method adapted from ASTM E96. The method of measurement was adapted by the author to specifically target this class of films. Tables of results are presented below. Silk type was kept consistent as 33B, or “mid skid” silk, (MW 24-40 kDa, PD 1.8-2.3) for this IDF due to a larger data set. However, the same behavior documented in this IDF is present in 27P, or “low skid” silk (MW 13-16 kDa, PD 1.7-2.4) as well.

TABLE 70
WVP values of RSF/nanoclay films of various concentrations.
All measurements were taken at 35C and 50% RH. RSF type is
33B for all samples.

| | | Water Vapor | |
|---|---|---|---|
| | Percent | Permeance | |
| | Clay | [g/m²*Pa*24 h] | StDev |
| Silk Only | 0% | 0.515 | 0.035 |
| Clay Only | 100% | 0.804 | 0.017 |
| 2001 | 20% | 0.348 | 0.019 |
| | 33% | 0.309 | 0.010 |
| | 50% | 0.295 | 0.011 |
| | 66% | 0.260 | 0.036 |
| 1100 | 20% | 0.397 | 0.048 |
| | 33% | 0.352 | 0.013 |
| | 50% | 0.304 | 0.031 |
| | 66% | 0.293 | 0.005 |

TABLE 71
Effect of nanoclay content on WVP.

| | 2001% | 1100% |
|---|---|---|
| Percent Clay | Reduction | Reduction |
| 0% | 0.0 | 0.0 |
| 20% | −32.4 ± 3.7% | −23.0 ± 9.3% |
| 33% | −39.9 ± 1.9% | −31.6 ± 2.5% |
| 50% | −42.8 ± 2.1% | −41.1 ± 6.1% |
| 67% | 49.5 ± 7.1% | −43.1 ± 1.0% |
| 100% | +56.3 ± 3.3% | ND |

### Mechanism

Bentonite Clay refers to a class of Tetrahedral-Octahedral-Tetrahedral (TOT) minerals. The outer sheets of the clay are composed of a tetrahedral SiO₄ structure, while the inner sheet sandwiched between the tetrahedral layers is composed of octahedral alumina (Al₂O₃). The outer layer bears a negative charge resulting from cation substitutions in the tetrahedral silica structure. The clay is susceptible to swelling as water penetrates into the inner layers of the system. Each outer tetrahedral layer of the clay exhibits very low permeability to water vapor and gasses.

In solution, clay clusters break down to single nanoscale-thick layers. Upon drying, these nanoclay layers arrange to form a network of parallel sheets, as confirmed via SEM. Water vapor and other gasses can readily diffuse through the space between these clay sheets due to their irregular stacking and available free volume in the film.

In contrast, films cast with pure RSF exhibit a comparatively homogenous structure. As is typical with polymer films, pure RSF films contain amorphous, semicrystalline and crystalline regions, densely packed, and free volume space throughout the film.

SEM images of 1:1 RSF/nanoclay films confirms a composite network that maintains the layered pattern of the exfoliated clay distributed throughout the RSF. RSF/nanoclay films cast under neutral conditions appear to retain more of the layered structure, while the identical solution cast under acidic conditions appear to form more ribbonlike structures. This could be due to the stronger electrostatic attractive forces between the RSF and nanoclay that exist under acidic conditions, while interactions between RSF and nanoclay under neutral conditions are driven by ion-dipole forces between negatively charged functional groups in the RSF and the exchangeable cations in the nanoclay inner layer.

Due to the extremely low permeability of the nanoclay sheets, water vapor is forced to diffuse around the clay layers within the film. In contrast with a pure nanoclay film, RSF decreases the free volume between the nanoclay layers and further restricts the motion of water vapor. Through this stacking and filling structure, the RSF and the nanoclay create a more tortuous pathway for any diffusing molecules to follow and resultingly yield a less permeable film. Up to concentrations of 67%, clay content and permeability for a film of a given area density are inversely proportional. Beyond this concentration, the physical properties of RSF/nanoclay solutions begin to substantially differ, posing a challenge for repeatable film casting and measurement.

FTIR scans have been taken to investigate the conformation of silk in a silk/nanoclay composite film. Specifically, the amide I (1700-1600 cm⁻¹) and amide II (1600-1500 cm⁻¹) peaks were analyzed. In neutral conditions (pH of ˜7.0), increasing the relative nanoclay concentration decreased the relative silk beta sheet content and increased the random coil structure.

### Example 31: Stabilizers for Silk Compositions

Stabilizers for all compositions disclosure herein include:
KCl
NaCl
MgCl₂
CaCl₂)
PBS
Tris
Polysorbate 20
Polysorbate 80
Capryl Glucoside
Sucrose
Histidine (buffer)
Glycine (buffer)
Arginine

### Excipient Overview and Mechanisms

Ionic strength/Salts
   Directly influence conformational integrity and stability
   Have been shown to dramatically affect the amount of protein aggregation induced by temperature and agitation
   Anions>cations in terms of influence on protein structure
Buffering agents (pH)
   Maintain solution pH (which greatly impacts protein higher-order structure)
   Can have specific ion effects, impacting chemical and conformational stability.
Surfactants
   Outcompete/shield protein molecules from hydrophobic surfaces (like air-water interfaces), which prevents protein unfolding
   May directly interact with hydrophobic regions in protein molecules
Sugars/Osmolytes
   Stabilize proteins through preferential hydration (increasing the amount water molecules can associate with protein surface, increasing solubility)
   Protect protein structure during stressful events (like lyophilization)
Amino Acids
   Stabilize via a variety of mechanisms: preferential hydration, direct binding, buffering capacity, antioxidant properties

### REFERENCES

Abdel-Naby, W., Cole, B., Liu, A., Liu, J., Wan, P., Guaiquil, V. H., Schreiner, R., Infanger, D., Lawrence, B. D., & Rosenblatt, M. I. (2017). Silk-derived protein enhances corneal epithelial migration, adhesion, and proliferation. Investigative Ophthalmology and Visual Science, 58(3), 1425-1433. https://doi.org/10.1167/iovs.16-19957
Fitsialos, G., Chassot, A.-A., Turchi, L., Dayem, M. A., LeBrigand, K., Moreilhon, C., Meneguzzi, G., Busca, R., Mari, B., Barbry, P., & Ponzio, G. (2007). Transcriptional signature of epidermal keratinocytes subjected to in vitro scratch wounding reveals selective roles for ERK1/2, p38, and phosphatidylinositol 3-kinase signaling pathways. The Journal of Biological Chemistry, 282(20), 15090-15102. https://doi.org/10.1074fjbc.M606094200
Huang, C., Rajfur, Z., Borchers, C., Schaller, M. D., & Jacobson, K. (2003). JNK phosphorylates paxillin and regulates cell migration. Nature, 424(6945), 219-223. https://doi.org/10.1038/nature01745
Klemke, R. L., Cai, S., Giannini, A. L., Gallagher, P. J., de Lanerolle, P., & Cheresh, D. A. (1997). Regulation of cell motility by mitogen-activated protein kinase. The Journal of Cell Biology, 137(2), 481-492. https://doi.org/10.1083fjcb.137.2.481
Körner P. Hydrothermal Degradation of Amino Acids. ChemSusChem. 2021; 14(22):4947-57.
Lee, M.-H., Koria, P., Qu, J., & Andreadis, S. T. (2009). JNK phosphorylates beta-catenin and regulates adherens junctions. FASEB Journal: Official Publication of the Federation of American Societies for Experimental Biology, 23(11), 3874-3883. https://doi.org/10.1096/fj.08-117804
Martinez-Mora, C., Mrowiec, A., Garcia-Vizcaino, E. M., Alcaraz, A., Cenis, J. L., & Nicolás, F. J. (2012). Fibroin and sericin from Bombyx mori Silk stimulate cell migration through upregulation and phosphorylation of c-Jun. PLoS ONE, 7(7). https://doi.org/10.1371/journal.pone.0042271
Onder, O. C., Batool, S. R. & Nazeer, M. A. Self-assembled silk fibroin hydrogels: from preparation to biomedical applications. Mater. Adv. 3, 6920-6949 (2022).
Park, K.-J., Jin, H.-H. & Hyun, C.-K. Antigenotoxicity of peptides produced from silk fibroin. Process Biochem. 38, 411-418 (2002).
Pearson, G., Robinson, F., Beers Gibson, T., Xu, B. E., Karandikar, M., Berman, K., & Cobb, M. H. (2001). Mitogen-activated protein (MAP) kinase pathways: regulation and physiological functions. Endocrine Reviews, 22(2), 153-183. https://doi.org/10.1210/edrv.22.2.0428
Ruiz-Lozano, R. E., Hernandez-Camarena, J. C., Loya-Garcia, D., Merayo-Lloves, J., & Rodriguez-Garcia, A. (2021). The molecular basis of neurotrophic keratopathy: Diagnostic and therapeutic implications. A review. In Ocular Surface (Vol. 19). Elsevier Inc. https://doi.org/10.1016/j.jtos.2020.09.007 DBO 15M60.1 459
Sah, M. K. & Pramanik, K. Regenerated Silk Fibroin from B. mori Silk Cocoon for Tissue Engineering Applications. Int. J. Environ. Sci. Dev. 404-408 (2010)
Stupack, D. G., Cho, S. Y., & Klemke, R. L. (2000). Molecular signaling mechanisms of cell migration and invasion. Immunologic Research, 21(2-3), 83-88. https://doi.org/10.1385/IR:21:2-3:83
Vepari, C., & Kaplan, D. L. (2007). Silk as a biomaterial. Progress in Polymer Science (Oxford), 32(8-9), 991-1007. https://doi.org/10.1016/j.progpolymsci.2007.05.013

1-15. (canceled)

16. A composition comprising a plurality of fibroin heavy chain peptides or protein fragments, fibroin light chain peptides or protein fragments, and/or fibrohexamerin peptides or protein fragments, each comprising a plurality of amino acids selected from M, R, V, K, T, F, I, L, C, A, Q, Y, N, D, E, G, S, H, P, and W,
wherein the composition comprises at least one amino acid modification, substitution, and/or replacement of selected from:
an asparagine to aspartic acid modification, substitution, and/or replacement,
a glutamine to glutamic acid modification, substitution, and/or replacement, and
a methionine to methionine oxide modification, substitution, and/or replacement.

17-22. (canceled)

23. The composition of claim 16, wherein a fibroin heavy chain modification, substitution, and/or replacement is selected from Q58, M64, N68, N70, N77, M80, N93, M103, Q125, N132, Q139, Q275, N4191, Q5216, and N5262.

24-25. (canceled)

26. The composition of claim 16, wherein a fibroin light chain modification, substitution, and/or replacement is selected from N23, Q24, N28, M69, N105, N108, N118, N136, N138, Q149, N186, N200, Q202, N204, N240, N248, and Q255.

27-28. (canceled)

29. The composition of claim 16, wherein a fibrohexamerin modification, substitution, and/or replacement is selected from Q62, N93, M120, N149, N172, N174, and N202.

30. The composition of claim 16, wherein each modification, substitution, and/or replacement is independently ranging in the composition between about 1% to about 99%.

31. The composition of claim 30, wherein a % modification, substitution, and/or replacement is defined as (number of peptide or protein fragments comprising a modification, substitution, and/or replacement at a specific position, divided by the total number of peptide or protein fragments which include the specific position, whether comprising a modification, substitution, and/or replacement, or not)×100.

32. (canceled)

33. The composition of claim 16, the composition comprising one or more fractions.

34. The composition of claim 33, the plurality of peptides or protein fragments having a weight average molecular weight (Mw) selected from between about 1 kDa and about 20 kDa, from between about 20 kDa and about 40 kDa, from between about 40 kDa and about 60 kDa, from between about 60 kDa and about 80 kDa, from between about 80 kDa and about 100 kDa, from between about 100 kDa and about 120 kDa, from between about 120 kDa and about 140 kDa, or from between about 140 kDa and about 160 kDa, from between about 160 kDa and about 180 kDa, from between about 180 kDa and about 200 kDa, or from between about 200 kDa and about 250 kDa, and a polydispersity between 1 and about 1.7.

35. The composition of claim 33, the plurality of peptides or protein fragments having a weight average molecular weight (Mw) selected from between about 10 kDa and about 20 kDa, from between about 20 kDa and about 40 kDa, from between about 40 kDa and about 60 kDa, from between about 60 kDa and about 80 kDa, from between about 80 kDa and about 100 kDa, from between about 100 kDa and about 120 kDa, from between about 120 kDa and about 140 kDa, from between about 140 kDa and about 160 kDa, or from between about 160 kDa and about 180 kDa, and a polydispersity between 1 and about 1.1, or 1 and about 1.2.

36. The composition of claim 33, the plurality of peptides or protein fragments in a fraction having a weight average molecular weight (Mw) selected from between about 10 kDa and about 20 kDa, from between about 20 kDa and about 40 kDa, from between about 40 kDa and about 60 kDa, from between about 60 kDa and about 80 kDa, from between about 80 kDa and about 100 kDa, from between about 100 kDa and about 120 kDa, or from between about 120 kDa and about 140 kDa, and a polydispersity between 1 and about 1.1, or 1 and about 1.2.

37. The composition of claim 33, the plurality of peptides or protein fragments in a fraction having a weight average molecular weight (Mw) selected from between about 60 kDa and about 80 kDa, from between about 80 kDa and about 100 kDa, or from between about 100 kDa and about 120 kDa, and a polydispersity between 1 and about 1.1.

38. The composition of claim 33, the plurality of peptides or protein fragments in a fraction having a weight average molecular weight (Mw) selected from between about 10 kDa and about 20 kDa, from between about 20 kDa and about 40 kDa, from between about 40 kDa and about 60 kDa, from between about 60 kDa and about 80 kDa, from between about 80 kDa and about 100 kDa, or from between about 100 kDa and about 110 kDa, and a polydispersity between 1 and about 1.1, or 1 and about 1.2.

39. The composition of claim 33, the plurality of peptides or protein fragments in a fraction having a weight average molecular weight (Mw) selected from between about 60 kDa and about 80 kDa, from between about 80 kDa and about 100 kDa, from between about 100 kDa and about 120 kDa, or from between about 120 kDa and about 140 kDa, and a polydispersity between 1 and about 1.1.

40. The composition of claim 33, the plurality of peptides or protein fragments in a fraction having a weight average molecular weight (Mw) selected from between about 20 kDa and about 40 kDa, or from between about 40 kDa and about 60 kDa, and a polydispersity between 1 and about 1.1, or 1 and about 1.2.

41-44. (canceled)

45. The composition of claim 33, the plurality of peptides or protein fragments having a weight average molecular weight (Mw) selected from between about 40 kDa and about 60 kDa, from between about 60 kDa and about 80 kDa, from between about 80 kDa and about 100 kDa, from between about 100 kDa and about 120 kDa, from between about 120 kDa and about 140 kDa, from between about 140 kDa and about 160 kDa, from between about 160 kDa and about 180 kDa, from between about 180 kDa and about 200 kDa, or from between about 200 kDa and about 220 kDa, and a polydispersity between 1 and about 1.7.

46. The composition of claim 33, the plurality of peptides or protein fragments in a fraction having a weight average molecular weight (Mw) selected from between about 40 kDa and about 60 kDa, from between about 60 kDa and about 80 kDa, from between about 80 kDa and about 100 kDa, from between about 100 kDa and about 120 kDa, from between about 120 kDa and about 140 kDa, from between about 140 kDa and about 160 kDa, from between about 160 kDa and about 180 kDa, from between about 180 kDa and about 200 kDa, or from between about 200 kDa and about 210 kDa, and a polydispersity between 1 and about 1.2, or 1 and about 1.3.

47. The composition of claim 33, the plurality of peptides or protein fragments in a fraction having a weight average molecular weight (Mw) selected from between about 40 kDa and about 60 kDa, from between about 60 kDa and about 80 kDa, from between about 80 kDa and about 100 kDa, or from between about 100 kDa and about 110 kDa, and a polydispersity between 1 and about 1.1, or 1 and about 1.2

48. The composition of claim 33, the plurality of peptides or protein fragments in a fraction having a weight average molecular weight (Mw) selected from between about 60 kDa and about 80 kDa, from between about 80 kDa and about 100 kDa, from between about 100 kDa and about 120 kDa, from between about 120 kDa and about 140 kDa, from between about 140 kDa and about 160 kDa, from between about 160 kDa and about 180 kDa, from between about 180 kDa and about 200 kDa, or from between about 200 kDa and about 210 kDa, and a polydispersity between 1 and about 1.2, or 1 and about 1.3.

49-64. (canceled)


SEQUENCE LISTING (48 sequences)

SEQ ID NO: 1 - protein, 6 aa, synthetic construct
  REGION 1..6: note = Synthetic protein-like multiblock polymer hexapeptide
  SITE 6: note = MISC_FEATURE - X can be is alanine, tyrosine, valine, or serine
GAGAGX

SEQ ID NO: 2 - protein, 6 aa, synthetic construct
  REGION 1..6: note = Synthetic protein-like multiblock polymer hexapeptide
GAGAGS

SEQ ID NO: 3 - protein, 5 aa, synthetic construct
  REGION 1..5: note = Synthetic mammalian elastin
VPGVG

SEQ ID NO: 4 - protein, 5 aa, synthetic construct
  REGION 1..5: note = Synthetic mammalian elastin
GVGVP

SEQ ID NO: 5 - protein, 6 aa, synthetic construct
  REGION 1..6: note = Synthetic linker repetitve fragment
GAGAGS

SEQ ID NO: 6 - protein, 44 aa, synthetic construct
  REGION 1..44: note = Synthetic protein
SGFGPVANGGSGEASSESDFGSSGFGPVANASSGEASSESDFAG

SEQ ID NO: 7 - skipped

SEQ ID NO: 8 - protein, 9 aa, synthetic construct
  REGION 1..9: note = Synthetic pro-resilin elastomeric protein
GGRPSDTYG

SEQ ID NO: 9 - protein, 9 aa, synthetic construct
  REGION 1..9: note = Synthetic pro-resilin elastomeric protein
GGRPSSSYG

SEQ ID NO: 10 - protein, 35 aa, Araneus diadematus
GSSAAAAAAAASGPGGYGPENQGPSGPGGYGPGGP

SEQ ID NO: 11 - protein, 5 aa, synthetic construct
  REGION 1..5: note = Synthetic recombinant silk protein repetitve unit
GPGAS

SEQ ID NO: 12 - protein, 5 aa, synthetic construct
  REGION 1..5: note = Synthetic recombinant silk protein repetitve unit
GPGSG

SEQ ID NO: 13 - protein, 5 aa, synthetic construct
  REGION 1..5: note = Synthetic recombinant silk protein repetitve unit
GPGGY

SEQ ID NO: 14 - protein, 5 aa, synthetic construct
  REGION 1..5: note = Synthetic recombinant silk protein repetitve unit
GPGGP

SEQ ID NO: 15 - protein, 5 aa, synthetic construct
  REGION 1..5: note = Synthetic recombinant silk protein repetitve unit
GPGGA

SEQ ID NO: 16 - protein, 5 aa, synthetic construct
  REGION 1..5: note = Synthetic recombinant silk protein repetitve unit
GPGQQ

SEQ ID NO: 17 - protein, 5 aa, synthetic construct
  REGION 1..5: note = Synthetic recombinant silk protein repetitve unit
GPGGG

SEQ ID NO: 18 - protein, 5 aa, synthetic construct
  REGION 1..5: note = Synthetic recombinant silk protein repetitve unit
GPGQG

SEQ ID NO: 19 - protein, 5 aa, synthetic construct
  REGION 1..5: note = Synthetic recombinant silk protein repetitve unit
GPGGS

SEQ ID NO: 20 - protein, 5 aa, synthetic construct
  REGION 1..5: note = Synthetic recombinant silk protein repetitve unit
AAAAA

SEQ ID NO: 21 - protein, 6 aa, synthetic construct
  REGION 1..6: note = Synthetic recombinant silk protein repetitve unit
AAAAAA

SEQ ID NO: 22 - protein, 7 aa, synthetic construct
  REGION 1..7: note = Synthetic recombinant silk protein repetitve unit
AAAAAAA

SEQ ID NO: 23 - protein, 8 aa, synthetic construct
  REGION 1..8: note = Synthetic recombinant silk protein repetitve unit
AAAAAAAA

SEQ ID NO: 24 - protein, 9 aa, synthetic construct
  REGION 1..9: note = Synthetic recombinant silk protein repetitve unit
AAAAAAAAA

SEQ ID NO: 25 - protein, 10 aa, synthetic construct
  REGION 1..10: note = Synthetic recombinant silk protein repetitve unit
AAAAAAAAAA

SEQ ID NO: 26 - protein, 9 aa, synthetic construct
  REGION 1..9: note = Synthetic recombinant silk protein repetitve unit
GGRPSDTYG

SEQ ID NO: 27 - protein, 9 aa, synthetic construct
  REGION 1..9: note = Synthetic recombinant silk protein repetitve unit
GGRPSSSYG

SEQ ID NO: 28 - protein, 24 aa, synthetic construct
  REGION 1..24: note = Synthetic recombinant silk protein repetitve unit
GPYGPGASAAAAAAGGYGPGSGQQ

SEQ ID NO: 29 - protein, 35 aa, synthetic construct; identical to SEQ ID NO: 10
  REGION 1..35: note = Synthetic recombinant silk protein repetitve unit

SEQ ID NO: 30 - protein, 20 aa, synthetic construct
  REGION 1..20: note = Synthetic recombinant silk protein repetitve unit
GPGQQGPGQQGPGQQGPGQQ

SEQ ID NO: 31 - protein, 27 aa, synthetic construct
  REGION 1..27: note = Synthetic recombinant silk protein repetitve unit
GPGGAGGPYGPGGAGGPYGPGGAGGPY

SEQ ID NO: 32 - protein, 28 aa, synthetic construct
  REGION 1..28: note = Synthetic recombinant silk protein repetitve unit
GGTTIIEDLDITIDGADGPITISEELTI

SEQ ID NO: 33 - protein, 34 aa, synthetic construct
  REGION 1..34: note = Synthetic recombinant silk protein repetitve unit
PGSSAAAAAAAASGPGQGQGQGQGQGGRPSDTYG

SEQ ID NO: 34 - protein, 39 aa, synthetic construct
  REGION 1..39: note = Synthetic recombinant silk protein repetitve unit
SAAAAAAAAGPGGGNGGRPSDTYGAPGGGNGGRPSSSYG

SEQ ID NO: 35 - protein, 18 aa, synthetic construct
  REGION 1..18: note = Synthetic recombinant silk protein repetitve unit
GGAGGAGGAGGSGGAGGS

SEQ ID NO: 36 - protein, 30 aa, synthetic construct
  REGION 1..30: note = Synthetic recombinant silk protein repetitve unit
GPGGAGPGGYGPGGSGPGGYGPGGSGPGGY

SEQ ID NO: 37 - protein, 24 aa, synthetic construct
  REGION 1..24: note = Synthetic recombinant silk protein repetitve unit
GPYGPGASAAAAAAGGYGPGCGQQ

SEQ ID NO: 38 - protein, 24 aa, synthetic construct
  REGION 1..24: note = Synthetic recombinant silk protein repetitve unit
GPYGPGASAAAAAAGGYGPGKGQQ

SEQ ID NO: 39 - protein, 35 aa, synthetic construct
  REGION 1..35: note = Synthetic recombinant silk protein repetitve unit
GSSAAAAAAAASGPGGYGPENQGPCGPGGYGPGGP

SEQ ID NO: 40 - protein, 35 aa, synthetic construct
  REGION 1..35: note = Synthetic recombinant silk protein repetitve unit
GSSAAAAAAAASGPGGYGPKNQGPSGPGGYGPGGP

SEQ ID NO: 41 - protein, 8 aa, synthetic construct
  REGION 1..8: note = Synthetic modified amino acid sequence
GCGGGGGG

SEQ ID NO: 42 - protein, 8 aa, synthetic construct
  REGION 1..8: note = Synthetic modified amino acid sequence
GKGGGGGG

SEQ ID NO: 43 - protein, 14 aa, synthetic construct
  REGION 1..14: note = Synthetic modified amino acid sequence
GCGGSGGGGSGGGG

SEQ ID NO: 44 - protein, 13 aa, synthetic construct
  REGION 1..13: note = Synthetic modified amino acid sequence
GKGGGGGGSGGGG

SEQ ID NO: 45 - protein, 13 aa, synthetic construct
  REGION 1..13: note = Synthetic modified amino acid sequence
GCGGGGGGSGGGG

SEQ ID NO: 46 - protein, 33 aa, Araneus diadematus
GSSAAAAAAAASGPGQGQGQGQGQGGRPSDTYG

SEQ ID NO: 47 - protein, 39 aa, Araneus diadematus; identical to SEQ ID NO: 34

SEQ ID NO: 48 - protein, 30 aa, synthetic construct; identical to SEQ ID NO: 36
  REGION 1..30: note = Synthetic recombinant silk protein repetitive fragment